(19) United States
(12) Patent Application Publication
(10) Pub. No.: US 2024/0400584 A1
(43) Pub. Date: Dec. 5, 2024

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Watertown, MA (US); Peter Smith, Arlington, MA (US); Sudeep Prajapati, Somerville, MA (US); Allen T. Hopper, Lexington, MA (US); Stephan Vyskocil, Benesov (CZ)

(21) Appl. No.: 18/688,081

(22) PCT Filed: Aug. 30, 2022
(86) PCT No.: PCT/US2022/075708

§ 371 Date: Feb. 29, 2024

Related U.S. Application Data
(60) Provisional application No. 63/325,171, filed on Mar. 30, 2022.
(60) Provisional application No. 63/238,430, filed on Aug. 30, 2021.

(51) Int. Cl. C07D 519/00; A61K 31/436; A61K 31/438; A61K 31/439; A61K 31/454; A61K 31/4545; A61K 31/496; A61K 31/4985; A61K 31/501; A61K 31/506; A61K 31/519; A61K 31/537; C07D 405/14; C07D 471/04; C07D 471/08; C07D 491/052

(52) U.S. Cl. CPC C07D 519/00; A61K 31/436; A61K 31/438; A61K 31/439; A61K 31/454; A61K 31/4545; A61K 31/496; A61K 31/4985; A61K 31/501; A61K 31/506; A61K 31/519; A61K 31/537; C07D 405/14; C07D 471/04; C07D 471/08; C07D 491/052

(57) ABSTRACT

The present disclosure features compounds (I) and related compositions that, inter alia, modulate nucleic acid splicing, e.g., splicing of a pre-mRNA, as well as methods of use thereof.

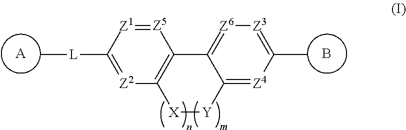

### CLAIM OF PRIORITY

This application claims priority to U.S. Application No. 63/238,430, filed on Aug. 30, 2021; and U.S. Application No. 63/325,171, filed on Mar. 30, 2022. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I) (e.g., a compound of Formulas (I-a), (I-b), (I-c), (I-d), (I-e), (I-f), (I-g) or (I-h)) and pharmaceutically acceptable salts, solvates, hydrates, tautomers, or stereoisomers thereof. The present disclosure additionally provides methods of using the compounds of the invention (e.g., compounds of Formulas (I), (I-a), (I-b), (I-c), (I-d), (I-e), (I-f), (I-g) or (I-h), and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (I-a), (I-b), (I-c), (I-d), (I-e), (I-f), (I-g), or (I-h), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (I-a), (I-b), (I-c), (I-d), (I-e), (I-f), (I-g) or (I-h), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (I-a), (I-b), (I-c), (I-d), (I-e), (I-f), (I-g) or (I-h), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

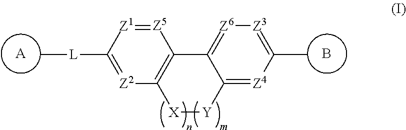

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, Z⁴, Z⁵, and Z⁶ are each independently C(R⁶) or N; X and Y are each independently O, C(R^{7a})(R^{7b}), or N(R^{7c}), wherein X and Y are not both O; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R¹²; each R^{S} is independently C₁-C₆-alkyl, C₁-C₆-heteroalkyl, C₁-C₆-haloalkyl, cycloalkyl, heterocyclyl, halo, cyano, oxo, —OR^{A}, —NR^{B}R^{C}, C(O)R^{D}, or —C(O)OR^{D}; R⁶ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —OR^{A}, —NR^{B}R^{C}, —C(O)R^{D}, or —C(O)OR^{D}; R^{7a}, R^{7b}, and R^{7c} are each independently hydrogen, C₁-C₆-alkyl, or halo; or R^{7a} and R^{7b}, together with the carbon atom to which they are attached, form an oxo group; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, or —OR^{A}; or R^{B} and R^{C} together with the atom to which they are attached form a 3-7-membered heterocyclyl or heteroaryl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, halo, cyano, —OR^{A}, —NR^{B}R^{C}, —NR^{B}C(O)R^{D}, —C(O)NR^{B}R^{C}, —C(O)R^{D}, —C(O)OR^{D}, or —C(O)R^{D}; each R^{A1} is hydrogen or C₁-C₆-alkyl; each of m and n is independently 1 or 2; and x is 0, 1, or 2.

In another aspect, the present invention provides pharmaceutical compositions comprising a compound of Formula (I) (e.g., a compound of Formulas (I-a), (I-b), (I-c), (I-d), (I-e), (I-f), (I-g) or (I-h)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I) (e.g., a compound of Formulas (I), (I-a), (I-b), (I-c), (I-d), (I-e), (I-f), (I-g) or (I-h)), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I) alters a target nucleic acid (e.g., DNA or RNA, e.g., a precursor RNA, e.g., a pre-mRNA), target protein, or combination thereof. In some embodiments, the compound of Formula (I) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue). In some embodiments, the presence of a compound of Formula (I) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue).

In another aspect, the present disclosure provides methods for preventing and/or treating a disease, disorder, or condition in a subject by administering a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I) (e.g., a compound of Formulas (I), (I-a), (I-b), (I-c), (I-d), (I-e), (I-f), (I-g) or (I-h))) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis.

In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I) (e.g., a compound of Formulas (I), (I-a), (I-b), (I-c), (I-d), (I-e), (I-f), (I-g) or (I-h)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I) (e.g., a compound of Formulas (I), (I-a), (I-b), (I-c), (I-d), (I-e), (I-f), (I-g) or (I-h))) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I) to a biological sample, a cell, or a subject comprises inhibition of cell growth or induction of cell death.

In another aspect, the present disclosure features kits comprising a container with a compound of Formula (I) (e.g., a compound of Formulas (I), (I-a), (I-b), (I-c), (I-d), (I-e), (I-f), (I-g) or (I-h)), or a pharmaceutically acceptable salt, solvate, hydrate, tautomer, stereoisomer thereof, or a pharmaceutical composition thereof. In certain embodiments, the kits described herein further include instructions for administering the compound of Formula (I) or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”).

In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl).

Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{c}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl.

Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 8 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means+10%. In certain embodiments, about means+5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

The present disclosure features a compound of Formula (I):

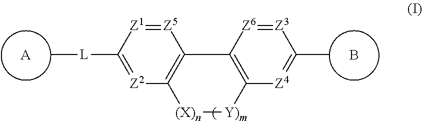

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —S—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, Z⁴, Z⁵, and Z⁶ are each independently C(R⁶) or N; X and Y are each independently O, C(R^{7a})(R^{7b}), or N(R^{7c}), wherein one of X and Y is independently C(R^{7a})(R^{7b}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R¹²; each R⁵ is independently C₁-C₆-alkyl, C₁-C₆-heteroalkyl, C₁-C₆-haloalkyl, cycloalkyl, heterocyclyl, halo, cyano, oxo, —OR^{A}, —NR^{B}R^{C}, C(O)R^{D}, or —C(O)OR^{D}; R⁶ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —OR^{A}, —NR^{B}R^{C}, —C(O)R^{D}, or —C(O)OR^{D}; R^{7a} and R^{7b} are each independently hydrogen, C₁-C₆-alkyl, or cycloalkyl; or R^{7a} and R^{7b}, together with the carbon atom to which they are attached, form an oxo group; R^{7c} is hydrogen or C₁-C₆-alkyl; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D}; each of R^{B} and R^{C} is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D}; or R^{B} and R^{C} together with the atom to which they are attached form a 3-7-membered heterocyclyl or heteroaryl ring optionally substituted with one or more R¹⁰; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R¹² is independently deuterium, halo, cyano, —OR^{A}, —NR^{B}R^{C}, —N^{B}C(O)R^{D}, —C(O)NR^{B}R^{C}, —C(O)R^{D}, —C(O)OR^{D}, or —C(O)R^{D}; each R^{A1} is hydrogen or C₁-C₆-alkyl; each of m and n is independently 1 or 2; and x is 0, 1, or 2.

As generally described herein, each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, either A or B is independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, either A or B is independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, either A or B is independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl. In some embodiments, A is monocyclic heterocyclyl and B is monocyclic heteroaryl or monocyclic heterocyclyl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹. In some embodiments, A is a nitrogen-containing heterocyclyl comprising 1 nitrogen atom and B is a nitrogen-containing heteroaryl or nitrogen-containing heterocyclyl comprising 1, 2, or 3 nitrogen atoms.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, each of A and B are independently selected from:

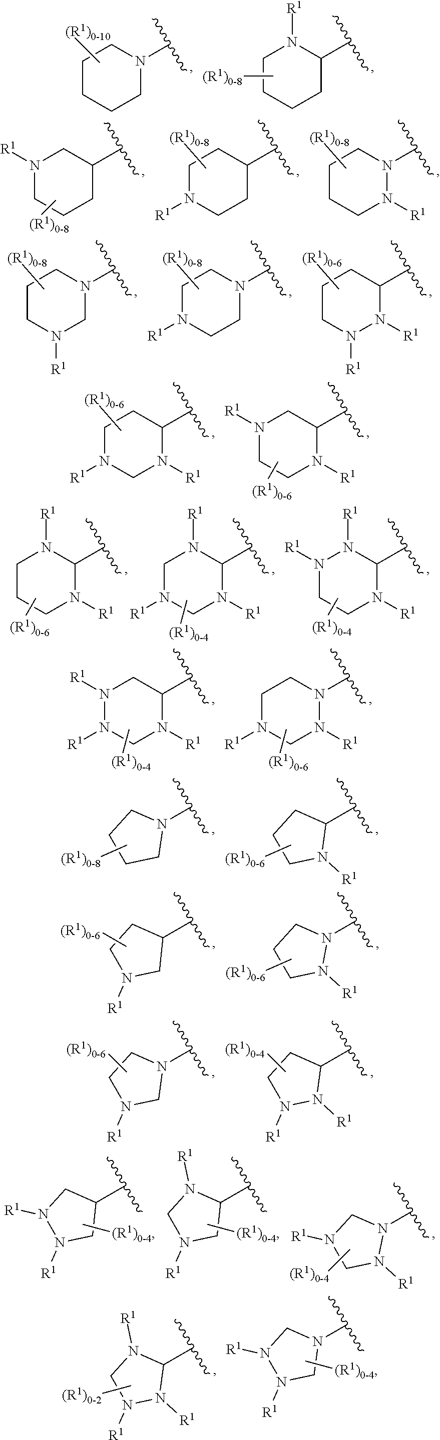

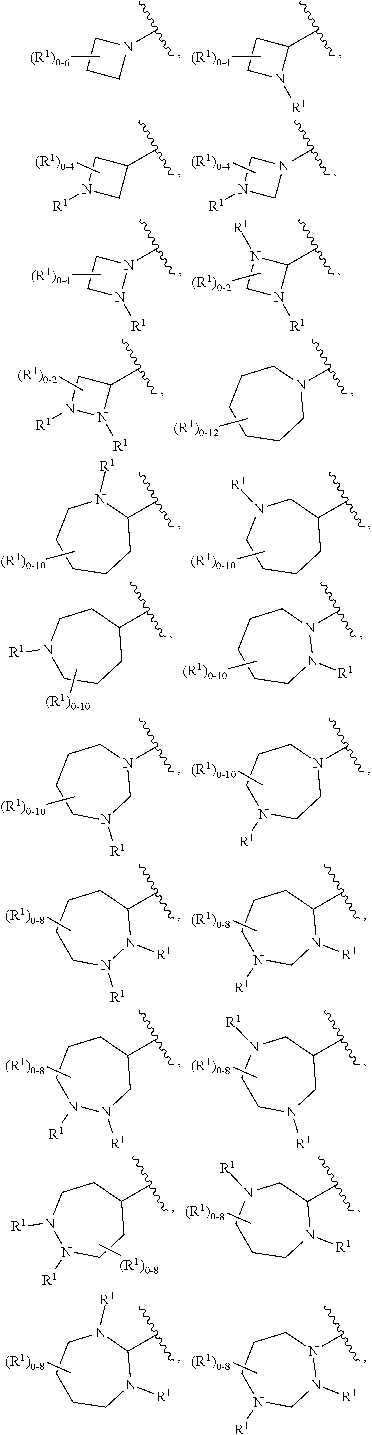

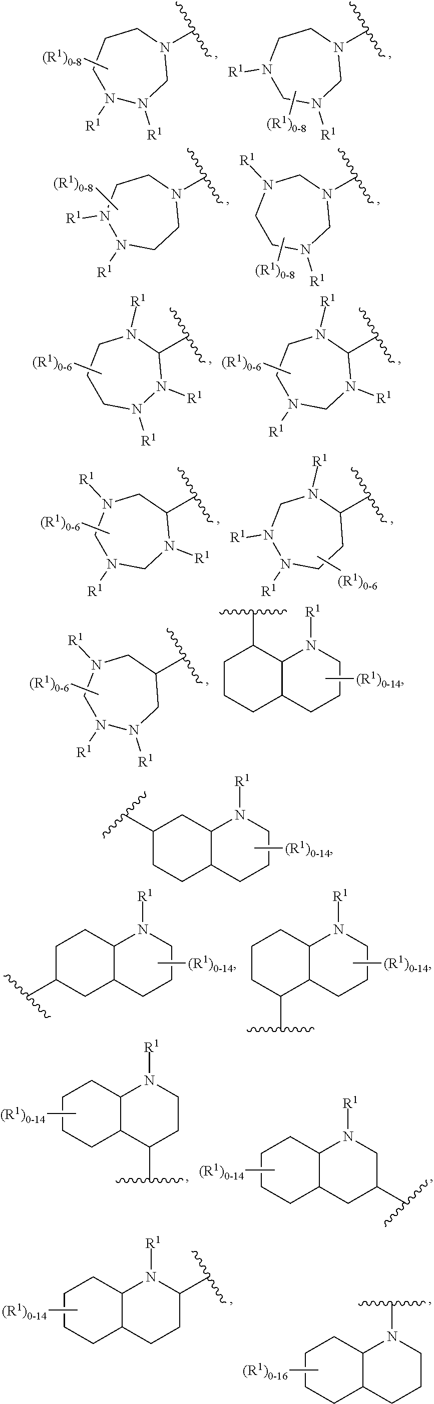

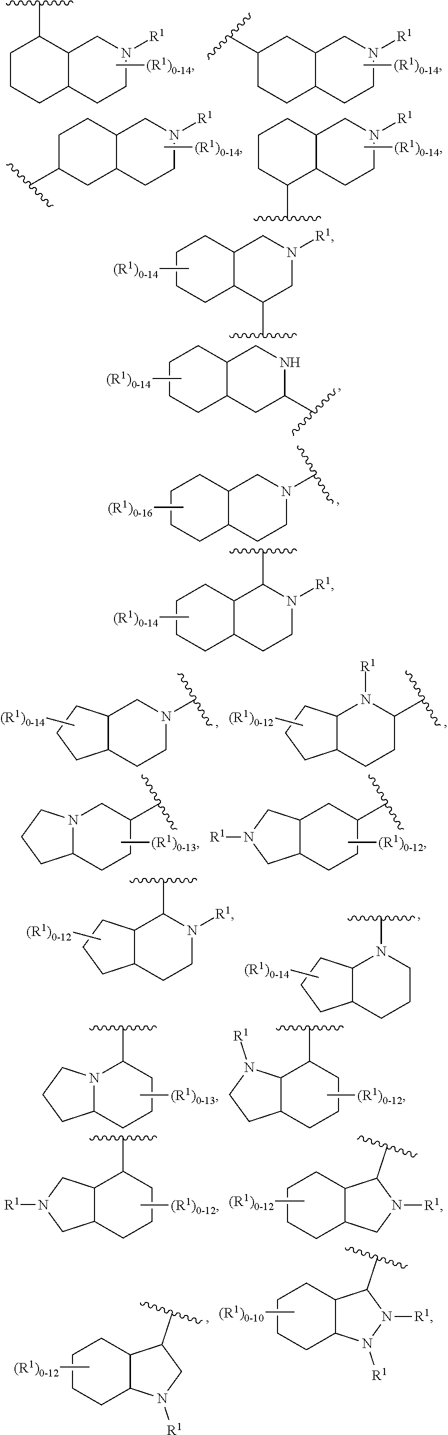

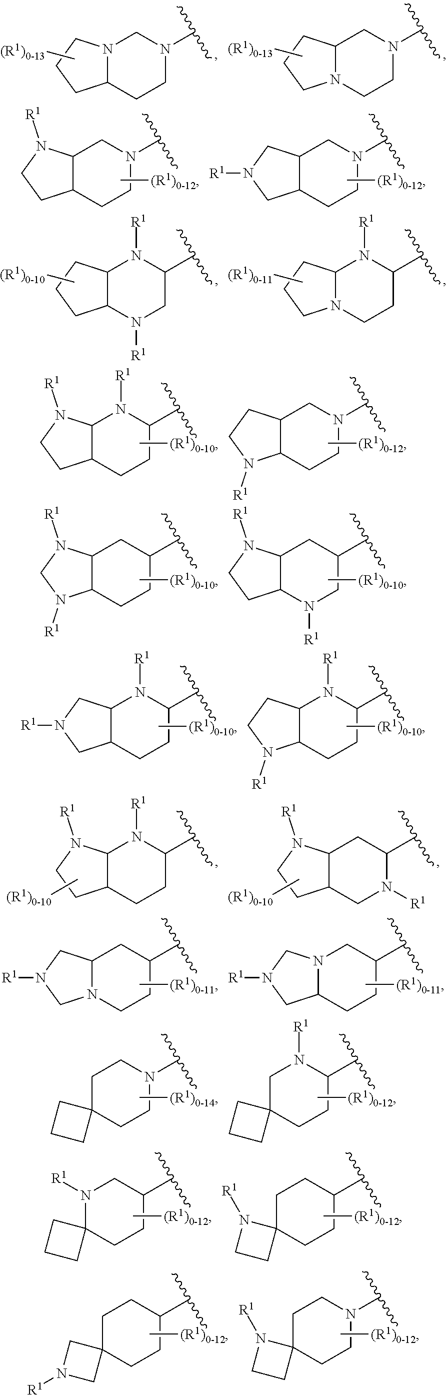

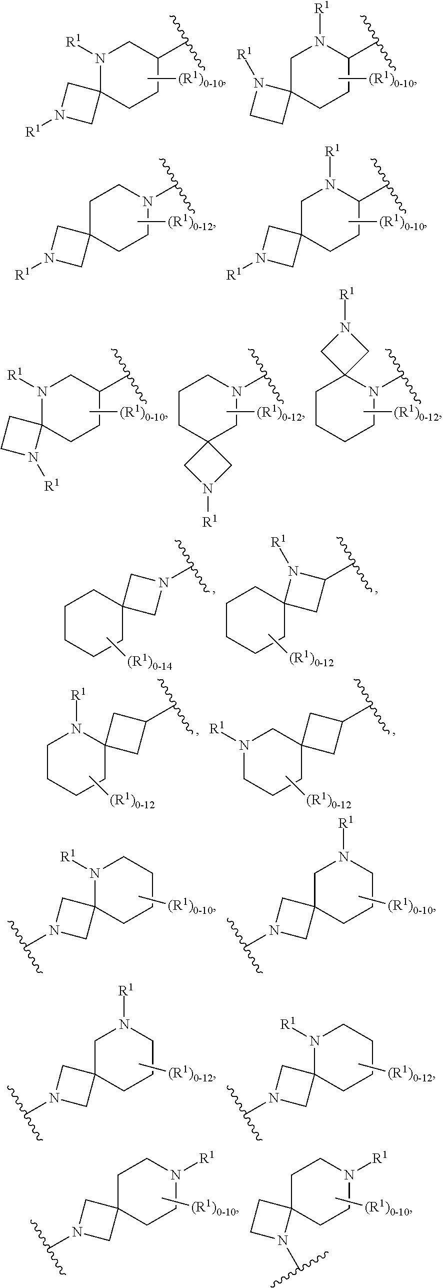

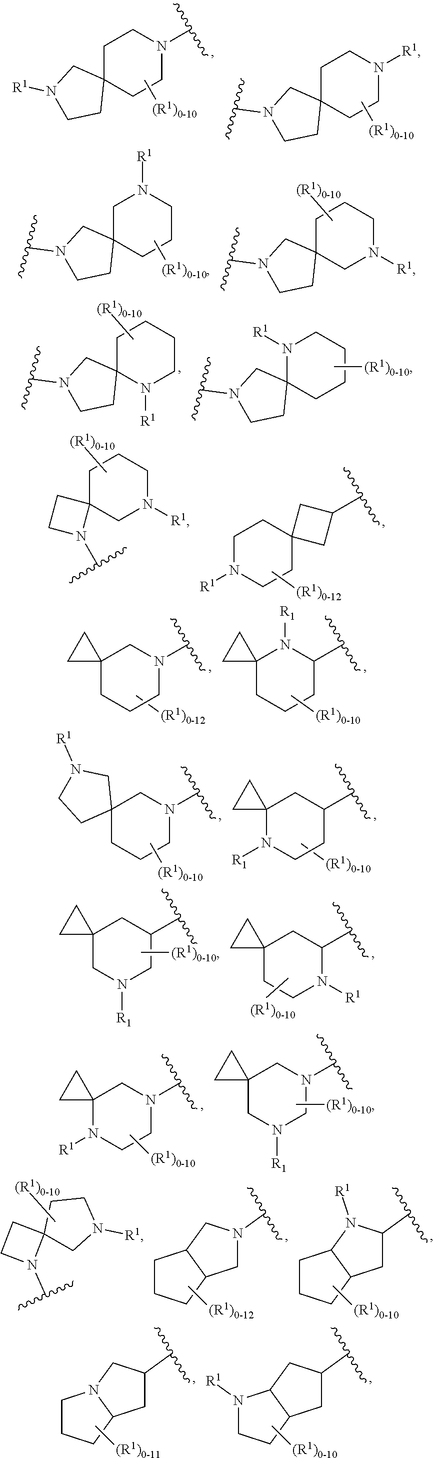

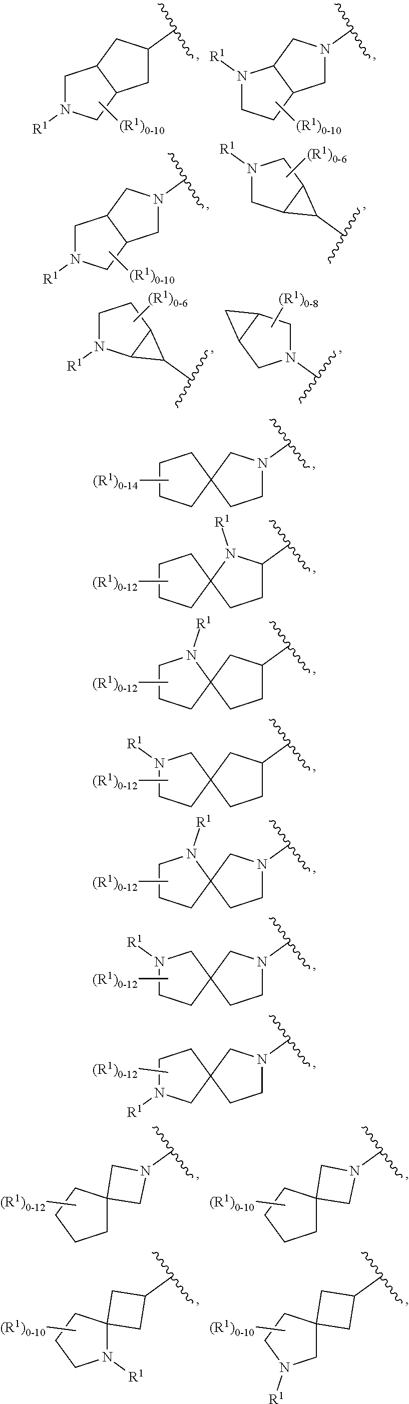

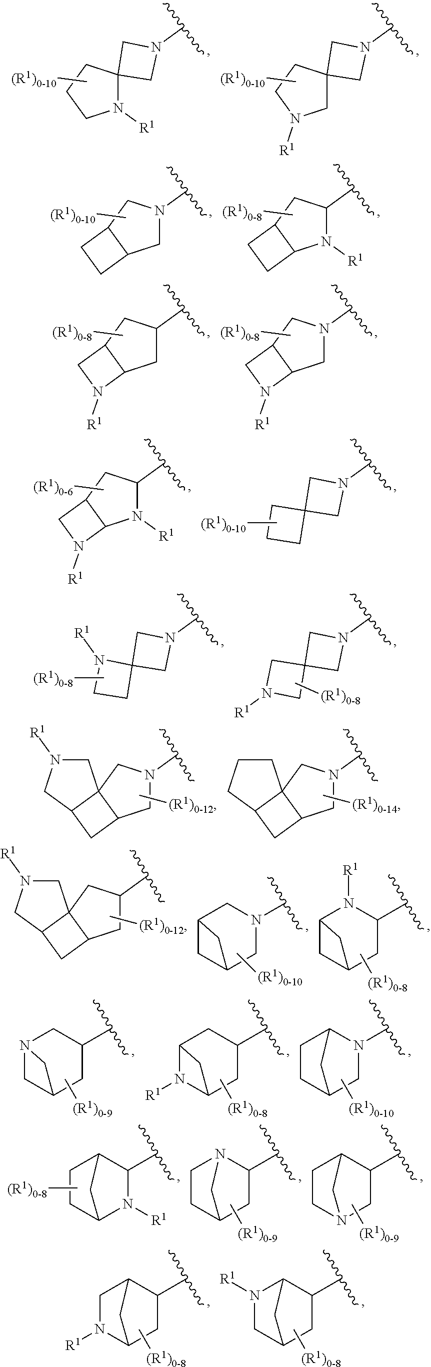

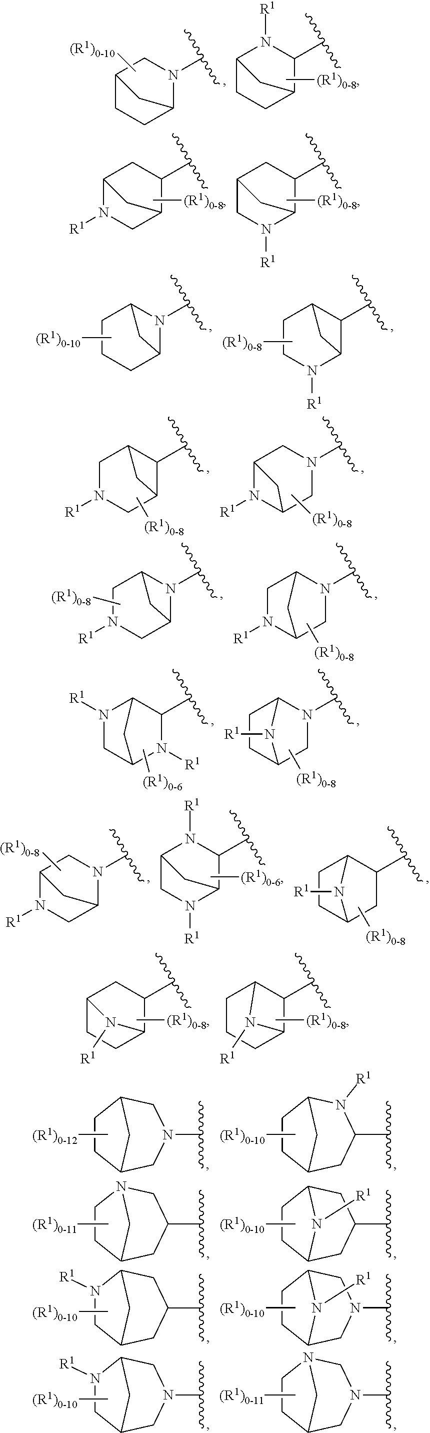

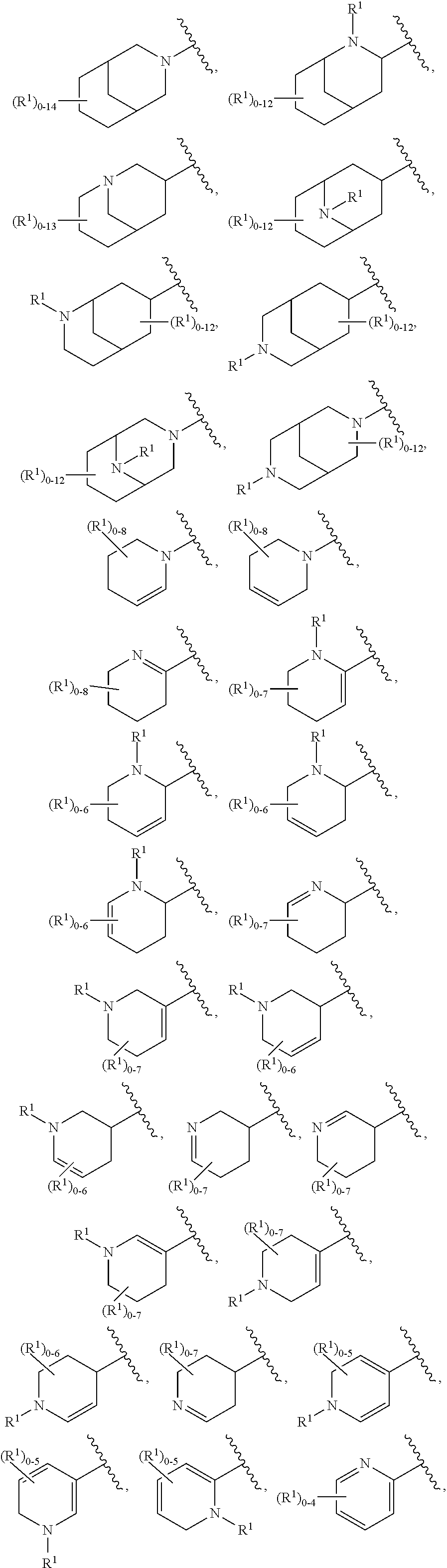

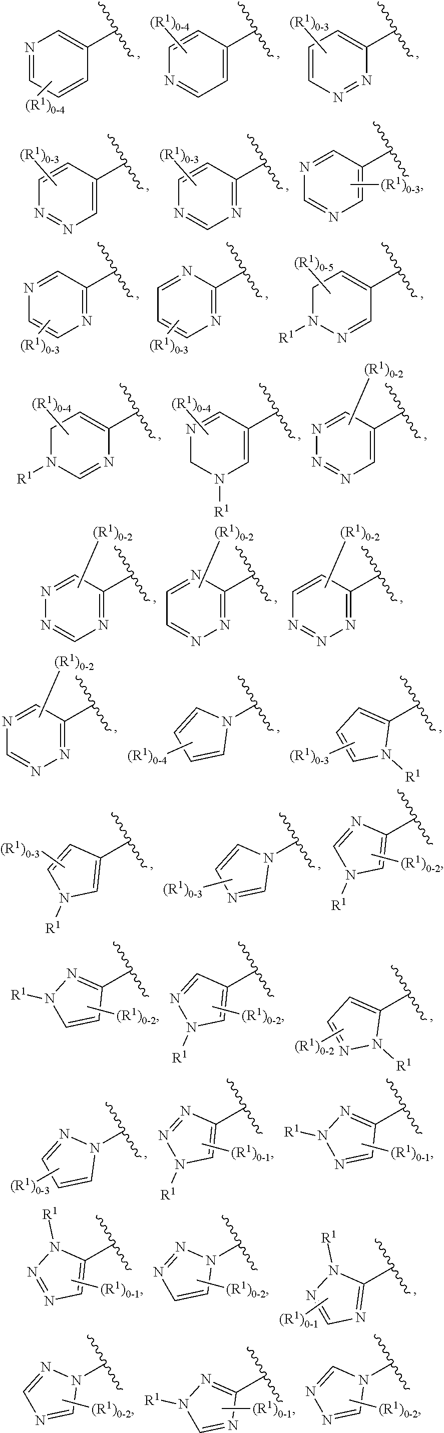

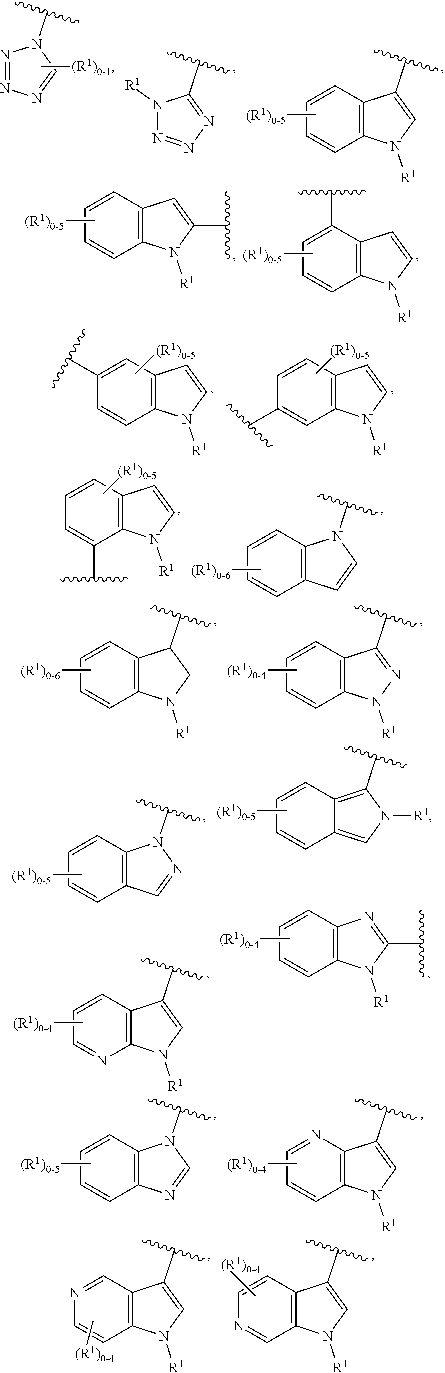

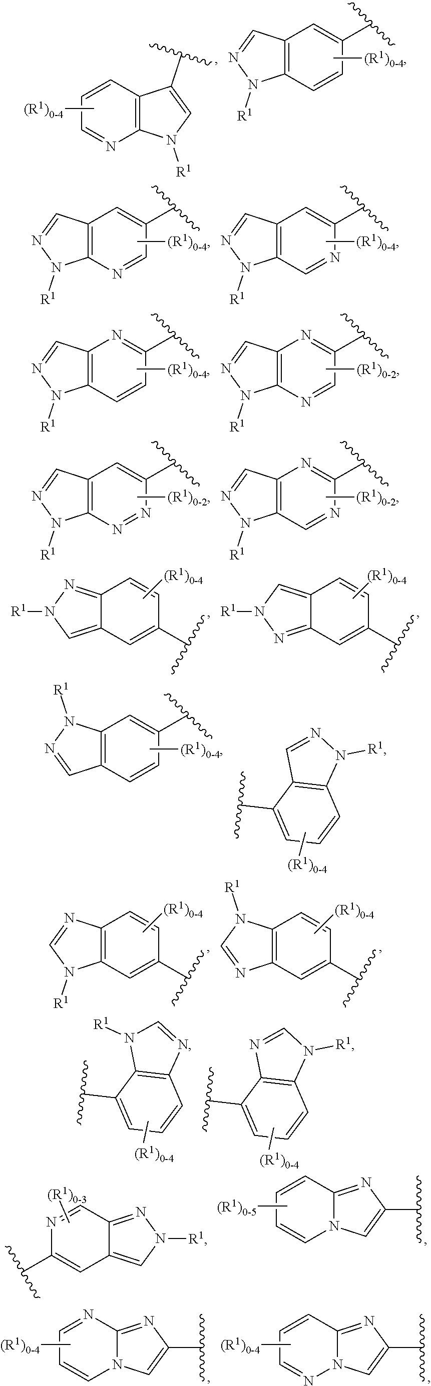

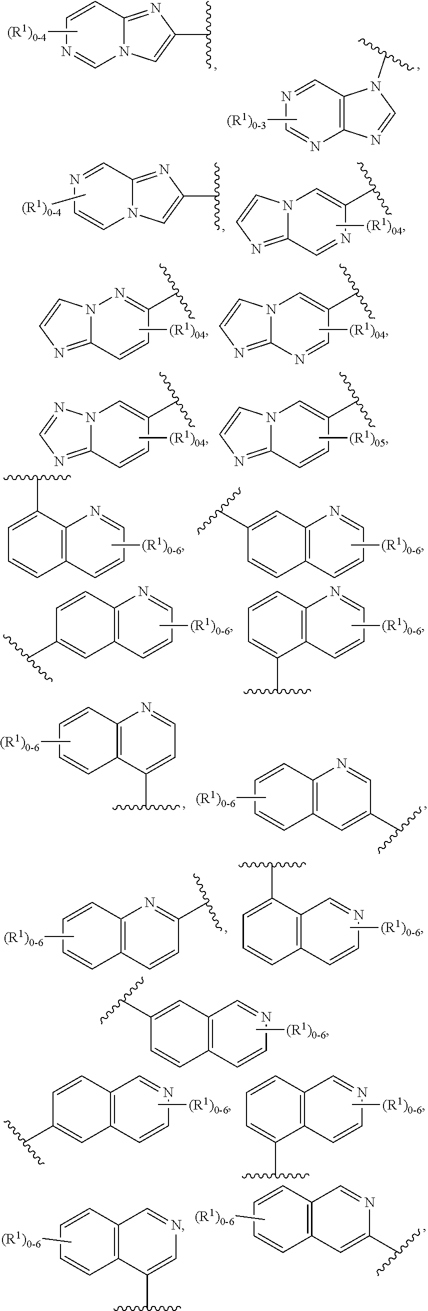

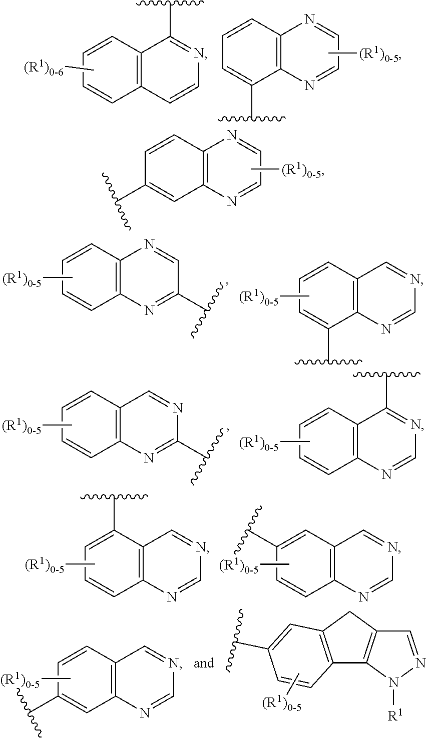

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

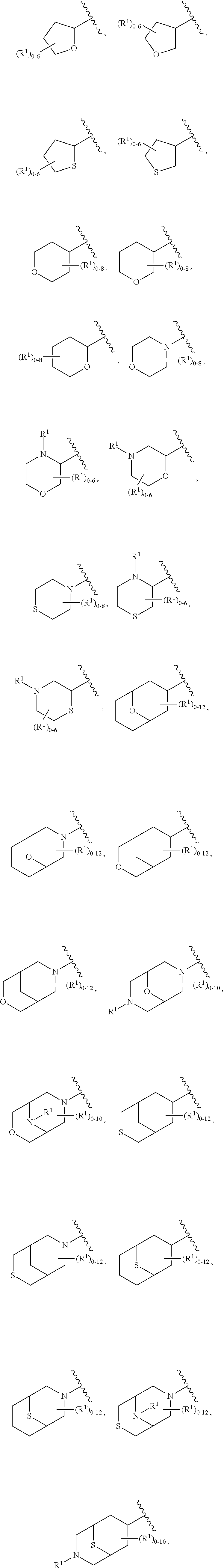

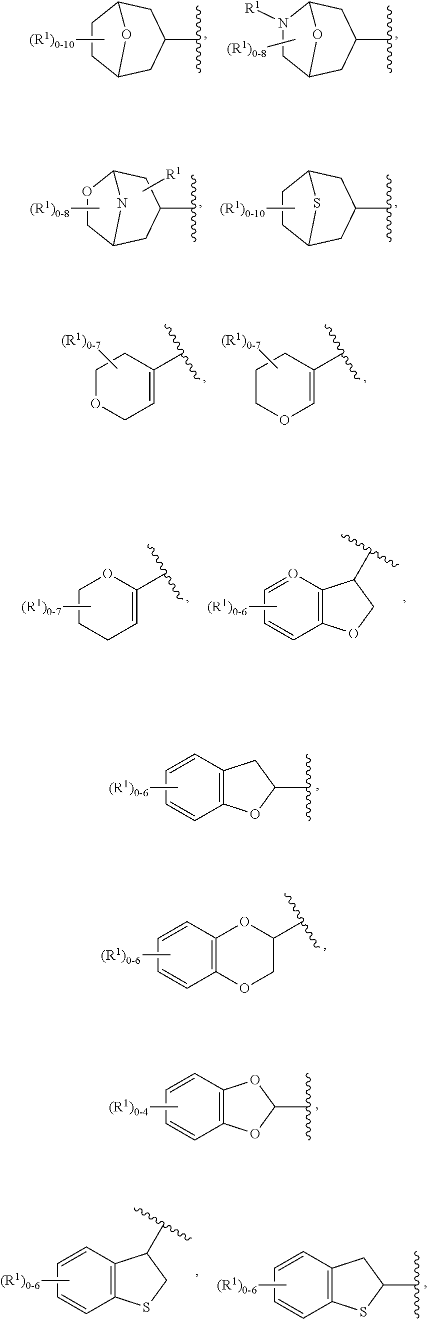

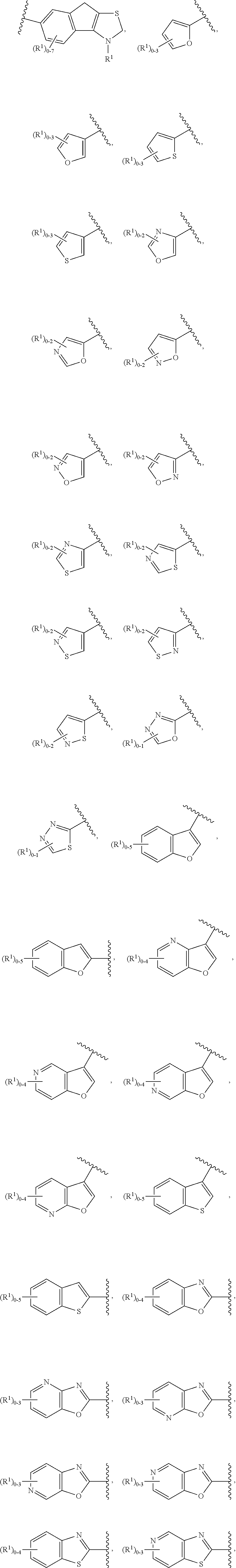

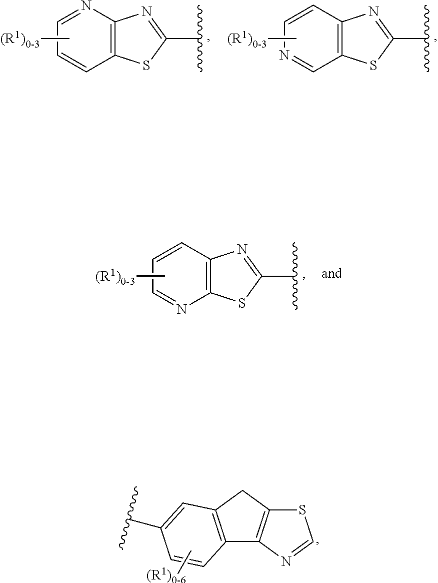

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B is independently heteroaryl or heterocyclyl, each of which is optionally substituted with one or more R¹. In some embodiments, one of A and B is independently a monocyclic heteroaryl or bicyclic heteroaryl, each of which is optionally substituted with one or more R¹. In some embodiments, one of A and B is independently a monocyclic heteroaryl optionally substituted with one or more R¹. In some embodiments, one of A and B is independently a nitrogen-containing heteroaryl optionally substituted with one or more R¹. In some embodiments, one of A and B is independently a 5-6 membered heteroaryl optionally substituted with one or more R¹

In some embodiments, one of A and B is independently selected from

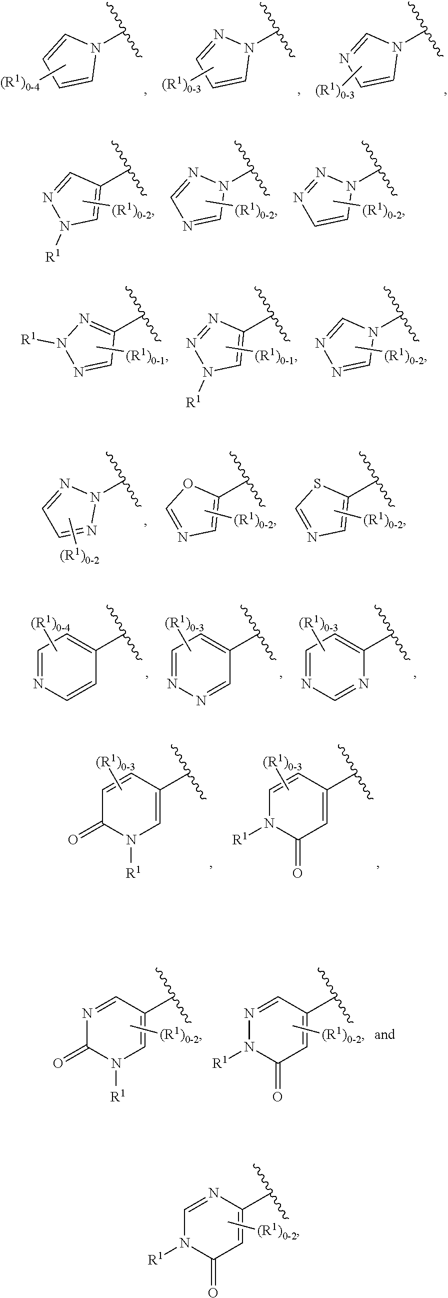

wherein R¹ is as described herein. In some embodiments, one of A and B is independently selected from

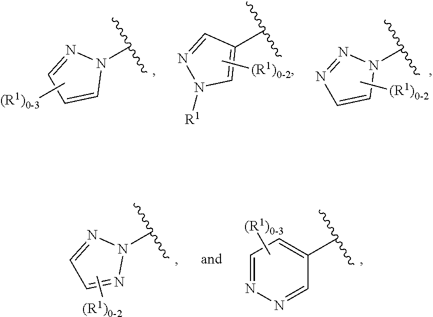

wherein R¹ is as described herein. In some embodiments, one of A and B is independently selected from

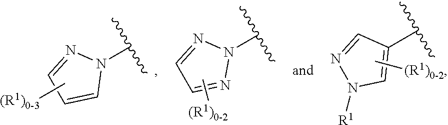

wherein R¹ is as described herein. In some embodiments, B is selected from

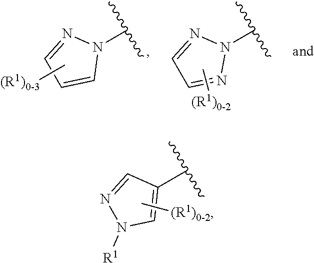

wherein R¹ is as described herein.

In some embodiments, one of A and B is independently selected from

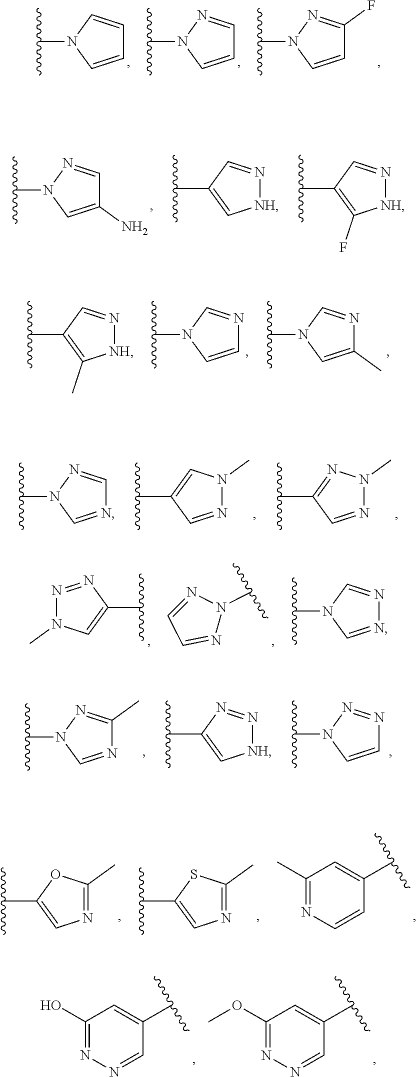

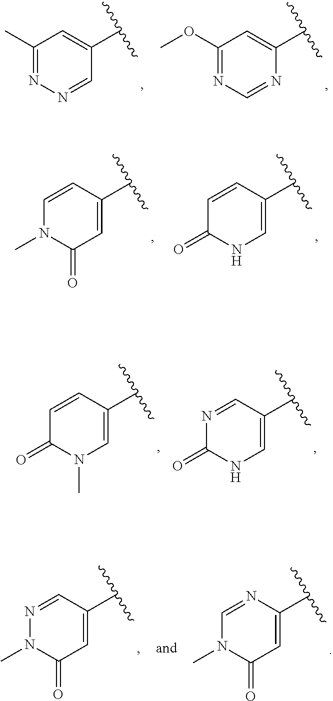

In some embodiments, one of A and B is independently selected from

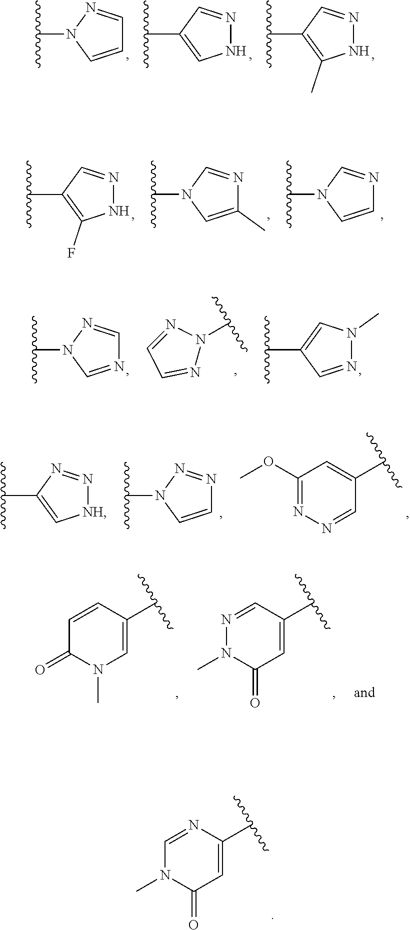

In some embodiments, one of A and B is independently selected from

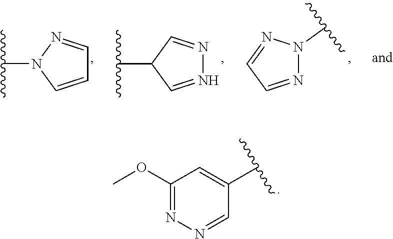

In some embodiments, one of A and B is independently

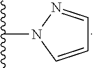 [SMILES: *n1cccn1]

In some embodiments, one of A and B is independently

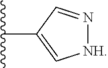 [SMILES: *c1cn[nH]c1]

In some embodiments, one of A and B is independently

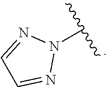 [SMILES: *n1nccn1]

In some embodiments, one of A and B is independently

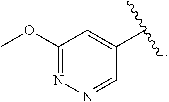 [SMILES: *c1cnnc(OC)c1]

In some embodiments, B is selected from

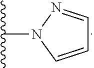 [SMILES: *n1cccn1]

In some embodiments, B is

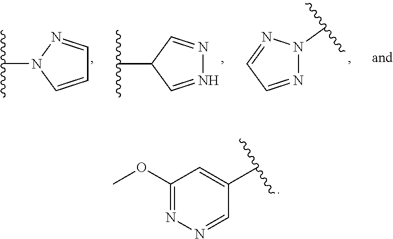

In some embodiments, B is

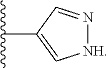 [SMILES: *c1cn[nH]c1]

In some embodiments, B is

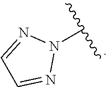 [SMILES: *n1nccn1]

In some embodiments, B is

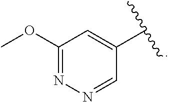 [SMILES: *c1cnnc(OC)c1]

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-10 membered heterocyclyl optionally substituted with one or more R¹.

In some embodiments, A is selected one of A and B is independently selected from

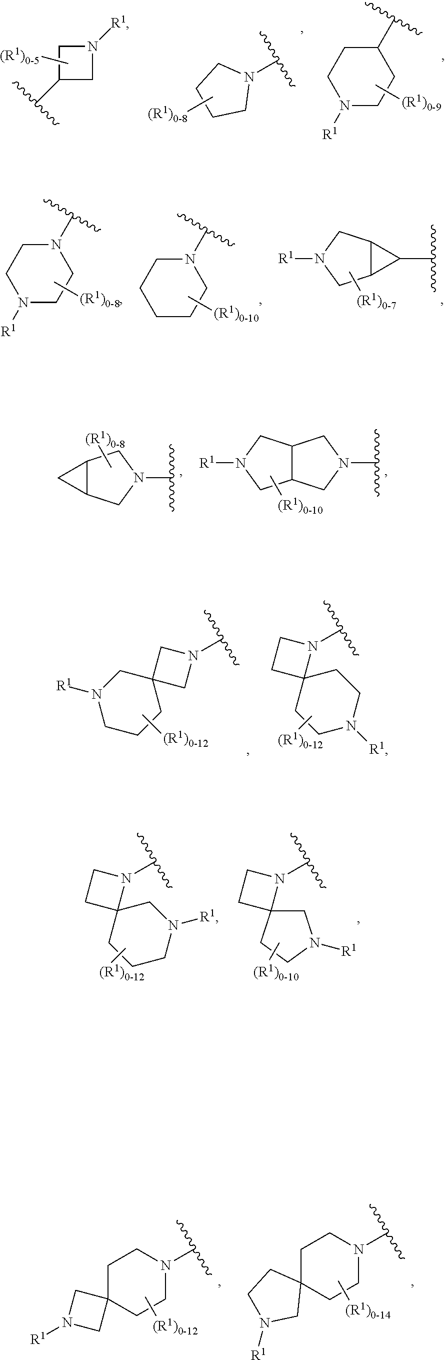

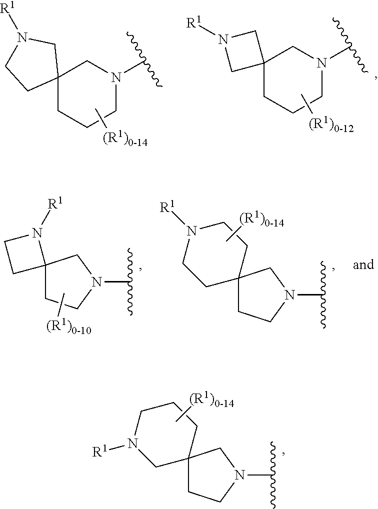

wherein R¹ is as described herein.

In some embodiments, one of A and B is independently selected from

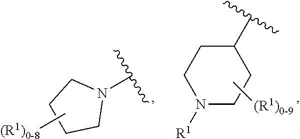

wherein R¹ is as defined herein. In some embodiments, one of A and B is independently selected from

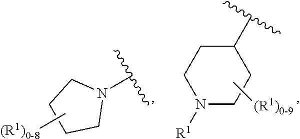

wherein R¹ is as defined herein. In some embodiments, A is

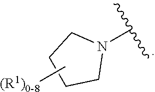

In some embodiments, A is

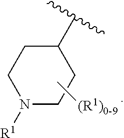

In some embodiments, one of A and B is independently selected from

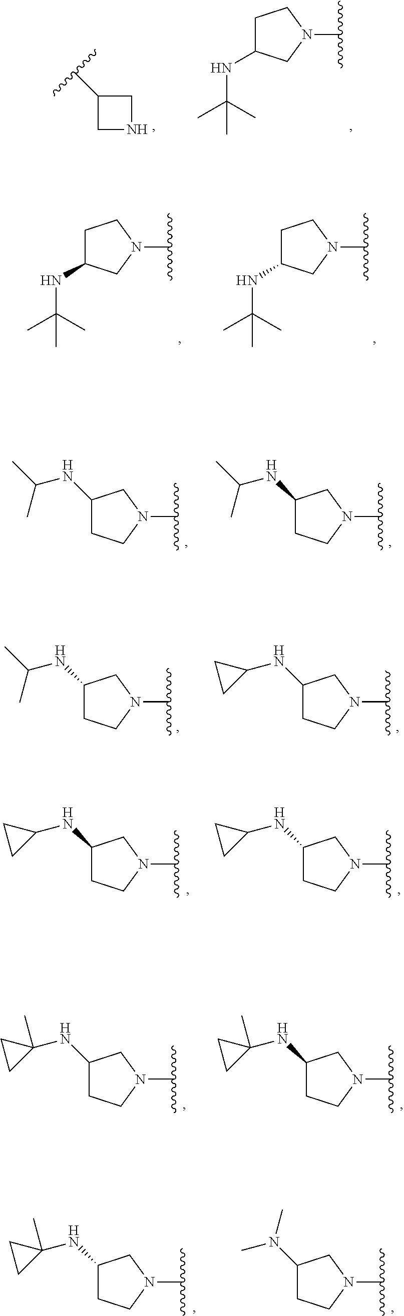

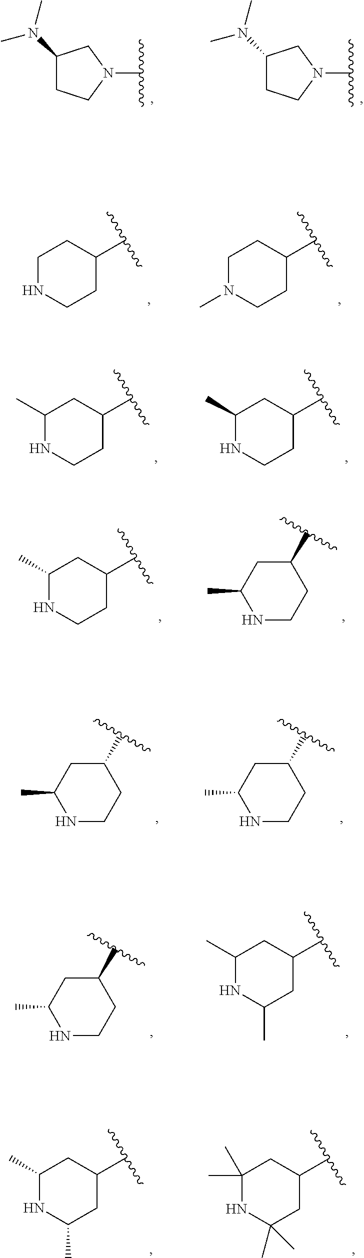

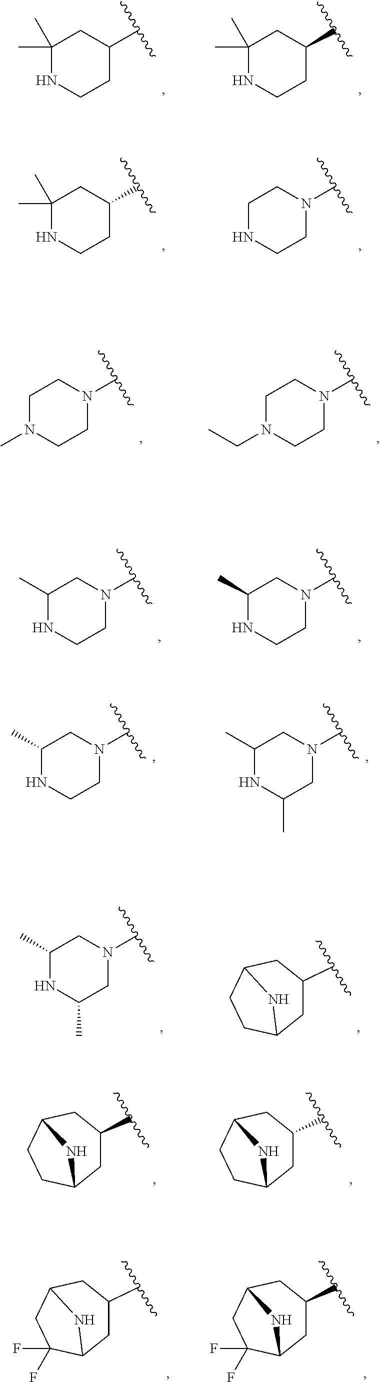

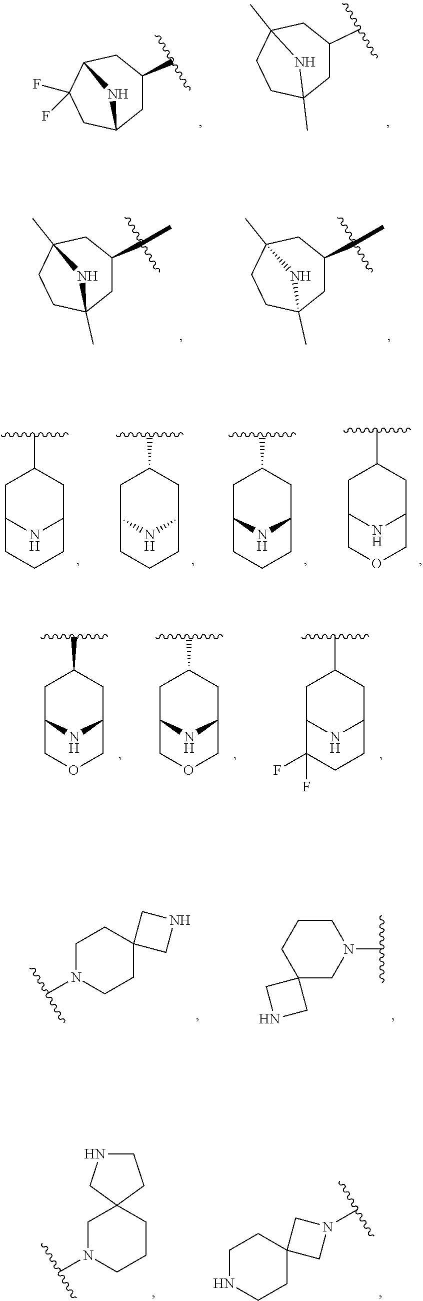

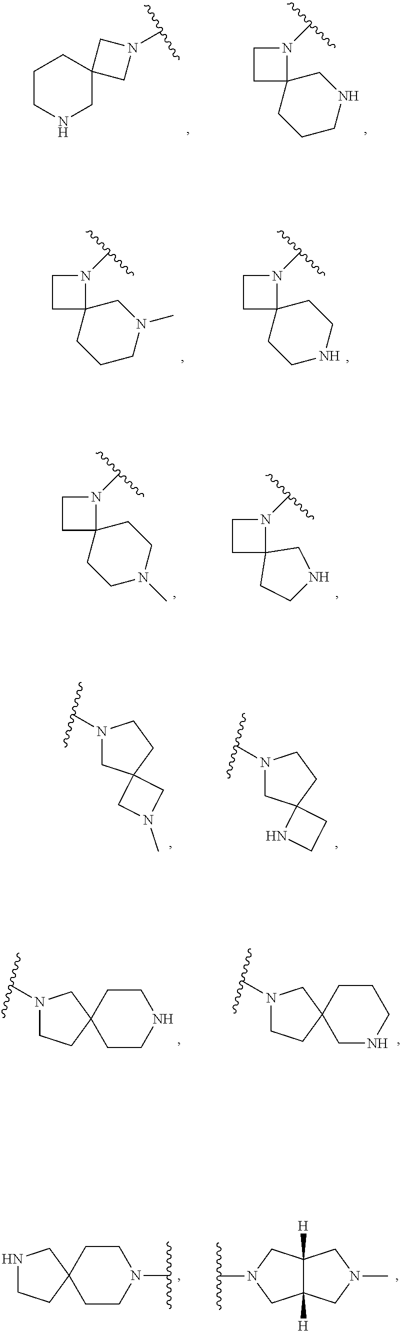

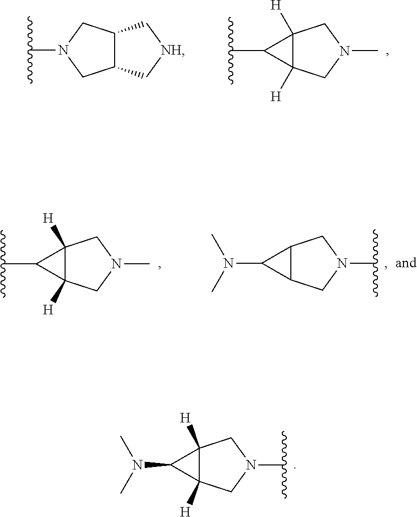

In some embodiments, one of A and B independently selected from

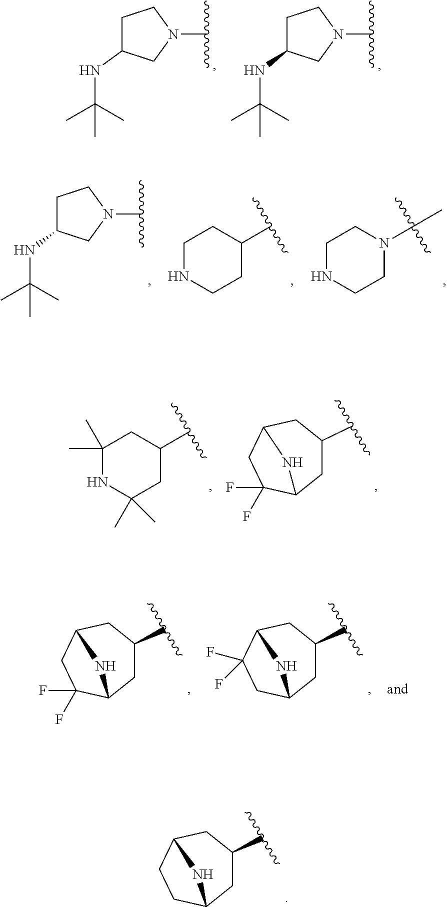

In some embodiments, one of A and B is independently selected from

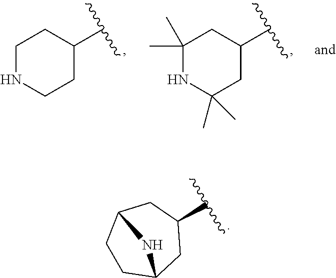 [SMILES: *C1CCNCC1 ; *C1CC(C)(C)NC(C)(C)C1 ; *[C@@H]1C[C@H]2CC[C@@H](C1)N2]

In some embodiments, one of A and B is

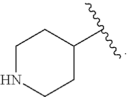 [SMILES: *C1CCNCC1]

In some embodiments, one of A and B is

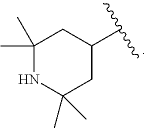 [SMILES: *C1CC(C)(C)NC(C)(C)C1]

In some embodiments, one of A and B is

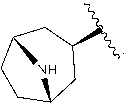 [SMILES: *[C@@H]1C[C@H]2CC[C@@H](C1)N2]

In some embodiments, A is selected from

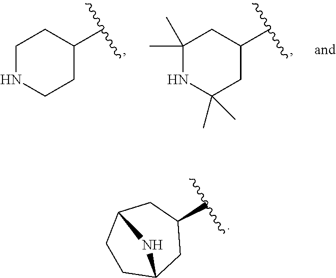 [SMILES: *C1CCNCC1 ; *C1CC(C)(C)NC(C)(C)C1 ; *[C@@H]1C[C@H]2CC[C@@H](C1)N2]

In some embodiments, A is

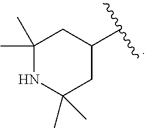 [SMILES: *C1CC(C)(C)NC(C)(C)C1]

In some embodiments, A is

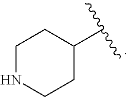 [SMILES: *C1CCNCC1]

In some embodiments, A is

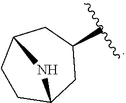 [SMILES: *[C@@H]1C[C@H]2CC[C@@H](C1)N2]

As generally described herein, L may be absent or refer to a C₁-C₆-alkylene, C₁-C₆-heteroalkylene, —O—, —S—, —C(O)—, —N(R⁴)—, —N(R⁴)C(O)—, or —C(O)N(R⁴)— group, wherein each alkylene and heteroalkylene is optionally substituted with one or more R⁵. In some embodiments, L is absent or C₁-C₆-alkylene, C₁-C₆-heteroalkylene, —O—, —C(O)—, —N(R⁴)—, —N(R⁴)C(O)—, or —C(O)N(R⁴)— group, wherein each alkylene and heteroalkylene is optionally substituted with one or more R⁵.

In some embodiments, L is absent. In some embodiments, L is C₁-C₆-alkylene (e.g., C₁-alkylene, C₂-alkylene, C₃-alkylene, C₄-alkylene, C₅-alkylene, or C₆-alkylene). In some embodiments, L is unsubstituted C₁-C₆ alkylene. In some embodiments, L is C₁-C₆-alkylene substituted with one or more R⁵. In some embodiments, L is C₁-alkylene substituted with one R⁵. In some embodiments, L is —CH₂— (or methylene). In some embodiments, L is —C(O)— (or carbonyl).

In some embodiments, L is absent, C₁-C₆-alkylene, C₁-C₆-heteroalkylene, —N(R⁴)C(O)—, or —C(O)N(R⁴)—, wherein each alkylene and heteroalkylene is optionally substituted with one or more R⁵.

In some embodiments, L is C₁-C₆-heteroalkylene (e.g., C₁-heteroalkylene, C₂-heteroalkylene, C₃-heteroalkylene, C₄-heteroalkylene, C₅-heteroalkylene, or C₆-heteroalkylene). In some embodiments, L is unsubstituted C₁-C₆ heteroalkylene. In some embodiments, L is C₁-C₆ heteroalkylene substituted with one or more R⁵. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L is —N(R⁴)C(O)—. In some embodiments, L is —C(O)N(R⁴)—.

In some embodiments, L is oxygen. In some embodiments, L is nitrogen, which may be substituted with R⁴. In some embodiments, L is nitrogen substituted with one R⁴. In some embodiments, L is —N(R⁴)—. In some embodiments, R⁴ is hydrogen, C₁-C₆ alkyl (e.g., CH₃ or CD₃), or cycloalkyl (e.g., cyclopropyl). In some embodiments, R⁴ is C₁-C₆ alkyl (e.g., CH₃), or cycloalkyl (e.g., cyclopropyl) substituted with one or more R¹² (e.g., deuterium). In some embodiments, L is —N(CH₃)—. In some embodiments, L is —NH—. In some embodiments, L is —O—. In some embodiments, L is —S—.

As generally described herein, Z¹, Z², Z³, Z⁴, Z⁵, and Z⁶ are each independently C(R⁶) or N. In some embodiments, Z¹, Z², Z³, Z⁴, Z⁵, and Z⁶ are each independently C(R⁶). In some embodiments, R⁶ is hydrogen. In some embodiments, one of Z¹, Z², Z³, Z⁴, Z⁵, and Z⁶ is independently N and the other of Z¹, Z², Z³, Z⁴, Z⁵, and Z⁶ are independently C(R⁶) (e.g., CH). In some embodiments, each of Z¹, Z², Z³, and Z⁴ independently refers to C(R⁶) (e.g., CH) or N.

In some embodiments, Z¹ and Z² are each independently C(R⁶), e.g., CH. In some embodiments, Z³ and Z⁴ are each independently C(R⁶), e.g., CH. In some embodiments, one of Z¹ and Z² is C(R⁶), and the other of Z¹ and Z² is N. In some embodiments, one of Z³ and Z⁴ is C(R⁶), and the other of Z³ and Z⁴ is N. In some embodiments, Z¹ is C(R⁶). In some embodiments, Z¹ is N. In some embodiments, Z² is C(R⁶). In some embodiments, Z² is N. In some embodiments, Z³ is C(R⁶). In some embodiments, Z³ is N. In some embodiments, Z⁴ is C(R⁶). In some embodiments, Z⁴ is N. In some embodiments, Z⁵ is C(R⁶). In some embodiments, Z⁵ is N. In some embodiments, Z⁶ is C(R⁶). In some embodiments, Z⁶ is N.

In some embodiments, Z² is N and each of Z¹, Z³, Z⁴, Z⁵, and Z⁶ is independently C(R⁶). In some embodiments, each of Z² and Z⁵ is independently N and each of Z¹, Z³, Z⁴, and Z⁶ is independently C(R⁶). In some embodiments, each of Z² is independently N and each of Z¹, Z³, Z⁴, Z⁵, and Z⁶ is independently C(R⁶). In some embodiments, Z¹ is C(R⁶) (e.g., CH) and Z² is N. In some embodiments, Z¹ is N and Z² is C(R⁶) (e.g., CH). In some embodiments, each of Z² and Z³ is independently N.

In some embodiments,

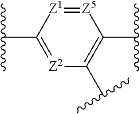

is selected from

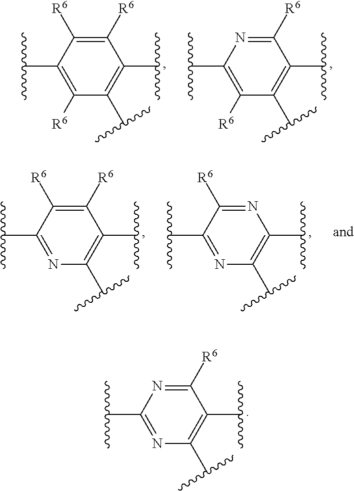

In some embodiments,

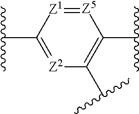

is selected from

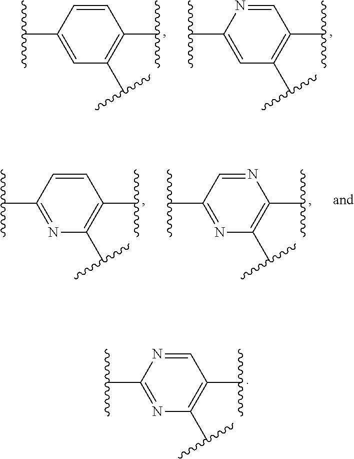

In some embodiments,

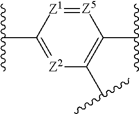

is

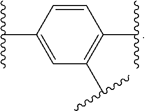 [SMILES: *c1ccc(*)c(*)c1]

In some embodiments,

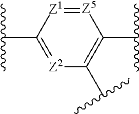

is

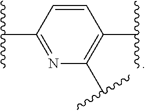 [SMILES: *c1ccc(*)c(*)n1]

In some embodiments,

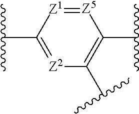

is

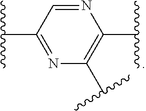 [SMILES: *c1cnc(*)c(*)n1]

In some embodiments,

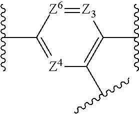

is selected from

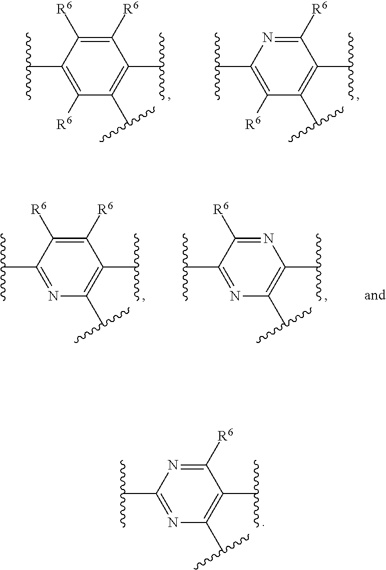

In some embodiments,

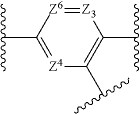

is

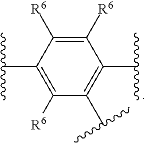

As generally described herein, each of X and Y independently refer to O, C(R^{7a})(R^{7b}), or N(R^{7c}). In some embodiments, one of X and Y is C(R^{7a})(R^{7b}), and the other of X and Y is O. In some embodiments, one of X and Y is C(R^{7a})(R^{7b}), and the other of X and Y is N(R^{7c}). In some embodiments, X is C(R^{7a})(R^{7b}) (e.g., —CH₂—). In some embodiments, X is O. In some embodiments, X is N(R^{7c}). In some embodiments, Y is C(R^{7a})(R^{7b}) (e.g., —CH₂—). In some embodiments, Y is O. In some embodiments, Y is N(R^{7c}). In some embodiments, X is O and Y is C(R^{7a})(R^{7I}) (e.g., —CH₂—). In some embodiments, X is O and Y is —CH₂—. In some embodiments, X is C(R^{7a})(R^{7b}) (e.g., —CH₂—) and Y is O. In some embodiments, X is —CH₂— and Y is O. In some embodiments, X is N(R^{7c}) (e.g., N(CH₃) and Y is C(R^{7a})(R^{7b}) (e.g., —CH₂—). In some embodiments, X is N(CH₃) and Y is —CH₂—. In some embodiments, Y is N(R^{7c}) (e.g., N(CH₃) and X is C(R^{7a})(R^{7b}) (e.g., —CH₂—). In some embodiments, Y is N(CH₃) and X is —CH₂—.

In some embodiments,

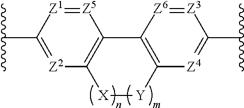

is selected from

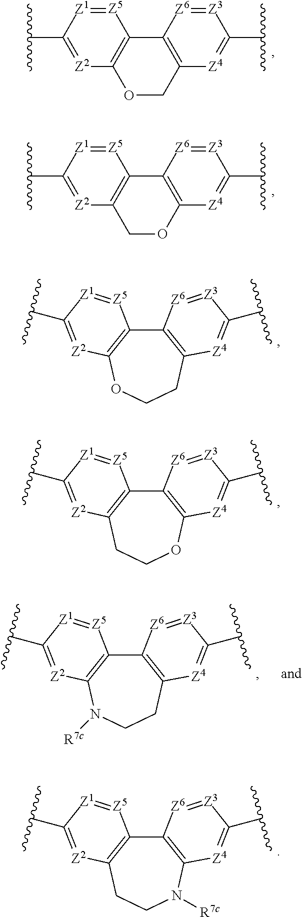

In some embodiments,

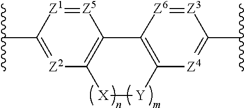

is selected from

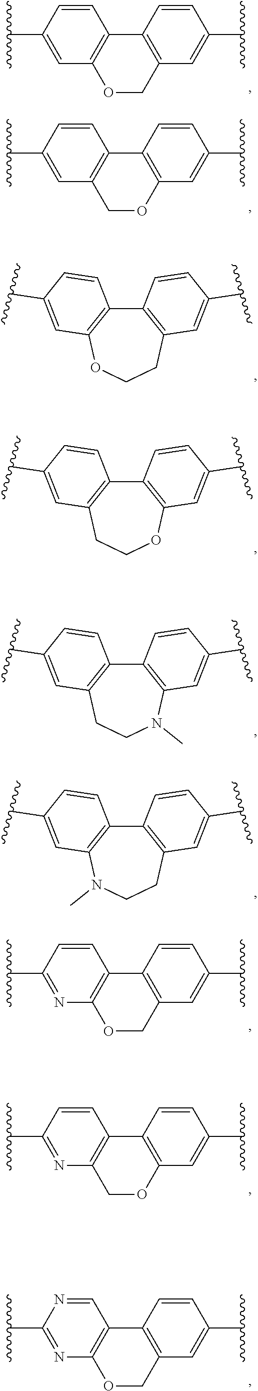

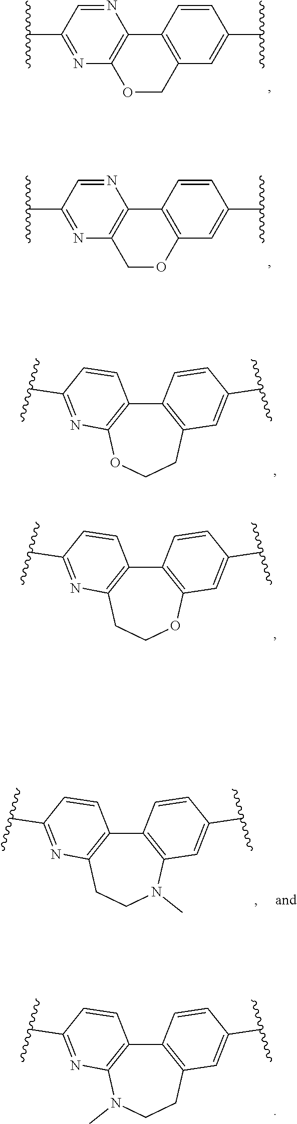 [SMILES: *c1ccc2c(c1)COc1nc(*)cnc1-2 ; *c1ccc2c(c1)OCc1nc(*)cnc1-2 ; *c1ccc2c(c1)CCOc1nc(*)ccc1-2 ; *c1ccc2c(c1)OCCc1nc(*)ccc1-2 ; *c1ccc2c(c1)N(C)CCc1nc(*)ccc1-2 ; *c1ccc2c(c1)CCN(C)c1nc(*)ccc1-2]

In some embodiments,

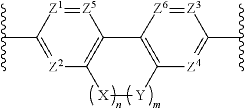

is selected from

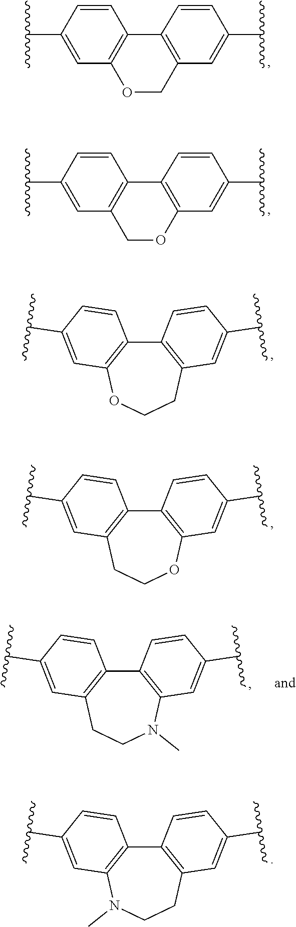 [SMILES: *c1ccc2c(c1)COc1cc(*)ccc1-2 ; *c1ccc2c(c1)COc1cc(*)ccc1-2 ; *c1ccc2c(c1)CCOc1cc(*)ccc1-2 ; *c1ccc2c(c1)CCOc1cc(*)ccc1-2 ; *c1ccc2c(c1)CCN(C)c1cc(*)ccc1-2 ; *c1ccc2c(c1)CCN(C)c1cc(*)ccc1-2]

In some embodiments,

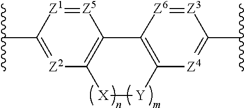

is selected from

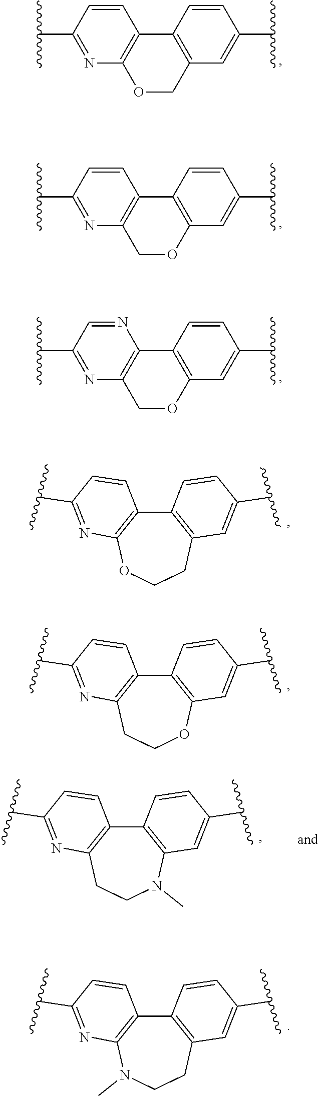

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁸. In some embodiments, R¹ is C₂-C₆-alkenyl substituted with one or more R⁸. In some embodiments, R¹ is C₂-C₆-alkynyl substituted with one or more R⁸. In some embodiments, R¹ is C₁-C₆-heteroalkyl substituted with one or more R⁸. In some embodiments, R¹ is C₁-C₆-haloalkyl substituted with one or more R⁸. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆-alkylene-aryl (e.g., benzyl). In some embodiments, R¹ is C₁-C₆-alkenylene-aryl. In some embodiments, R¹ is C₁-C₆-alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆-alkylene-aryl (e.g., benzyl), unsubstituted C₁-C₆-alkenylene-aryl (e.g., benzyl), unsubstituted C₁-C₆-alkylene-heteroaryl, or unsubstituted heteroaryl. In some embodiments, R¹ is cycloalkyl substituted with one or more R⁸. In some embodiments, R¹ is heterocyclyl substituted with one or more R⁸. In some embodiments, R¹ is aryl substituted with one or more R⁸. In some embodiments, R¹ is C₁-C₆-alkylene-aryl substituted with one or more R⁸. In some embodiments, R¹ is C₁-C₆-alkenylene-aryl substituted with one or more R⁸. In some embodiments, R¹ is C₁-C₆-alkylene-heteroaryl substituted with one or more R⁸. In some embodiments, R¹ is heteroaryl substituted with one or more R⁸.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is —C(O)NR^{B}R^{C}. In some embodiments, R¹ is —C(O)R^{D}. In some embodiments, R¹ is —C(O)OR^{D}. In some embodiments, R⁸ is —SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁸.

In some embodiments, each of R^{2a} and R^{2b} are independently hydrogen. In some embodiments, R^{2a} is hydrogen. In some embodiments, R^{2b} is hydrogen. In some embodiments, each of R^{2a} and R^{2b} are independently C₁-C₆-alkyl, C₂-C₆-alkenyl, or C₂-C₆-alkynyl. In some embodiments, R^{2a} is C₁-C₆-alkyl. In some embodiments, R^{2b} is C₁-C₆-alkyl. In some embodiments, R^{2a} is C₂-C₆-alkenyl. In some embodiments, R^{2b} is C₂-C₆-alkenyl. In some embodiments, R^{2a} is C₂-C₆-alkynyl. In some embodiments, R^{2b} is C₂-C₆-alkynyl. In some embodiments, R^{2a} is halo (e.g., fluoro, chloro, bromo, or iodo). In some embodiments, R^{2b} is halo (e.g., fluoro, chloro, bromo, or iodo). In some embodiments, R^{2a} is cyano. In some embodiments, R^{2b} is cyano. In some embodiments, R^{2a} is —OR^{A} (e.g., —OH). In some embodiments, R^{2b} is —OR^{A} (e.g., —OH).

In some embodiments, R⁴ is hydrogen. In some embodiments, R⁴ is C₁-C₆ alkyl. In some embodiments, R⁴ is C₁-alkyl (e.g., methyl). In some embodiments, R⁴ is C₁-C₆-haloalkyl (e.g., —CF₃ or —CHF₂). In some embodiments, R⁴ is methyl.

In some embodiments, R⁵ is C₁-C₆-alkyl. In some embodiments, R⁵ is C₁-C₆-heteroalkyl. In some embodiments, R⁵ is C₁-C₆-haloalkyl (e.g., —CF₃ or —CHF₂). In some embodiments, R⁵ is cycloalkyl. In some embodiments, R⁵ is halo (e.g., fluoro, chloro, bromo, or iodo). In some embodiments, R⁵ is cyano. In some embodiments, R⁵ is oxo. In some embodiments, R⁵ is —OR^{A}(e.g., —OH or —OMe). In some embodiments, R⁵ is —NR^{B}R^{C} (e.g., —NH₂ or —NMe₂). In some embodiments, R⁵ is —C(O)R^{D}. In some embodiments, R⁵ is —C(O)OR^{D}.

In some embodiments, R⁶ is hydrogen. In some embodiments, R⁶ is C₁-C₆ alkyl. In some embodiments, R⁶ is C₂-C₆-alkenyl. In some embodiments, R⁶ is C₂-C₆-alkynyl. In some embodiments, R⁶ is halo (e.g., fluoro, bromo, chloro, or iodo). In some embodiments, R⁶ is cyano. In some embodiments, R⁶ is —OR^{A} (e.g., —OH).

In some embodiments, R^{7a}, R^{7b}, and R^{7c} are each independently hydrogen or C₁-C₆-alkyl. In some embodiments, R^{7a} is hydrogen. In some embodiments, R^{7b} is hydrogen. In some embodiments, R^{7c} is hydrogen. In some embodiments, R^{7a} is C₁-C₆-alkyl. In some embodiments, R^{7b} is C₁-C₆-alkyl. In some embodiments, R⁷, is C₁-C₆-alkyl. In some embodiments, R^{7c} is methyl. In some embodiments, R^{7a} and R^{7b}, together with the carbon atom to which they are attached to form an oxo group.

In some embodiments, R⁸ is C₁-C₆-alkyl. In some embodiments, R⁸ is C₂-C₆-alkenyl. In some embodiments, R⁸ is C₂-C₆-alkynyl. In some embodiments, R⁸ is C₁-C₆-heteroalkyl. In some embodiments, R⁸ is C₁-C₆-haloalkyl (e.g., —CF₃ or —CHF₂). In some embodiments, R⁸ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁸ is C₁-C₆-alkyl substituted with one or more R¹¹. In some embodiments, R⁸ is C₂-C₆-alkenyl substituted with one or more R¹¹. In some embodiments, R⁸ is C₂-C₆-alkynyl substituted with one or more R¹¹. In some embodiments, R⁸ is C₁-C₆-haloalkyl substituted with one or more R¹¹. In some embodiments, R⁸ is C₁-C₆-heteroalkyl substituted with one or more R¹¹.

In some embodiments, R⁸ is cycloalkyl. In some embodiments, R⁸ is heterocyclyl. In some embodiments, R⁸ is aryl. In some embodiments, R⁸ is heteroaryl. In some embodiments, R⁸ is unsubstituted cycloalkyl, unsubstituted heterocyclyl, unsubstituted aryl, or unsubstituted heteroaryl. In some embodiments, R⁸ is cycloalkyl substituted with one or more R¹¹. In some embodiments, R⁸ is heterocyclyl substituted with one or more R¹¹. In some embodiments, R⁸ is aryl substituted with one or more R¹¹. In some embodiments, R⁸ is heteroaryl substituted with one or more R¹¹.

In some embodiments, R⁸ is halo (e.g., fluoro, chloro, bromo, or iodo). In some embodiments, R⁸ is cyano. In some embodiments, R⁸ is oxo. In some embodiments, R⁸ is —OR^{A}. In some embodiments, R⁸ is —NR^{B}R^{C}. In some embodiments, R⁸ is —NR^{B}C(O)R^{D}. In some embodiments, R⁸ is —NO₂. In some embodiments, R⁸ is —C(O)NR^{B}R^{C}. In some embodiments, R⁸ is —C(O)R^{D}. In some embodiments, R⁸ is —C(O)OR^{D}. In some embodiments, R⁸ is —SR^{E}. In some embodiments, R⁸ is —S(O)ₓR^{D}.

In some embodiments, R¹⁰ is C₁-C₆-alkyl. In some embodiments, R¹⁰ halo (e.g., fluoro, chloro, bromo, or iodo).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl. In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R¹⁰ (e.g., 1, 2, or 3 R¹⁰).

In some embodiments, R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, each of R^{D} and R^{E} is independently hydrogen. In some embodiments, R^{D} is independently C₁-C₆ alkyl. In some embodiments, R^{D} is hydrogen. In some embodiments, R^{D} is C₁-C₆ alkyl (e.g., methyl). In some embodiments, R^{D} is C₁-C₆ heteroalkyl. In some embodiments, R^{D} is C₁-C₆ haloalkyl. In some embodiments, R^{E} is C₁-C₆ haloalkyl. In some embodiments, R^{D} is cycloalkyl. In some embodiments, R^{D} is heterocyclyl. In some embodiments, R^{D} is aryl. In some embodiments, R^{D} is heteroaryl. In some embodiments, R^{D} is C₁-C₆ alkylene-aryl (e.g., benzyl). In some embodiments, R^{D} is C₁-C₆ alkylene-heteroaryl.

In some embodiments, R^{A1} is hydrogen. In some embodiments, R^{A1} is C₁-C₆ alkyl. In some embodiments, R^{A1} is C₁-alkyl (e.g., methyl). In some embodiments, R^{A1} is methyl.

In some embodiments, m is 1 or 2. In some embodiments, m is 1. In some embodiments, m is 2. In some embodiments, n is 1 or 2. In some embodiments, n is 1. In some embodiments, n is 2. In some embodiments, m and n are each 1. In some embodiments, n is 1 and m is 2. In some embodiments, n is 2 and m is 1. In some embodiments, x is an integer of 0, 1, or 2.

The present disclosure features a compound of Formula (I-a):

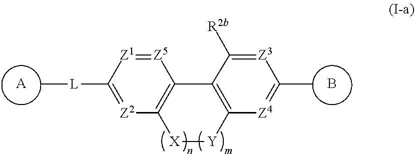

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, Z⁴, and Z⁵ are each independently C(R⁶) or N; X and Y are each independently O, C(R^{7a})(R^{7b}), or N(R^{7c}), wherein X and Y are not both O when n and m are both 1; R^{2b} is independently hydrogen, halo, cyano, C₁-C₆-alkyl, C₂-C₆-alkenyl, C₂-C₆-alkynyl, C₁-C₆-heteroalkyl, C₁-C₆-haloalkyl, or —OR^{A}; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R^{S} is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{7a}, R^{7b}, and R^{7c} are each independently hydrogen or C₁-C₆-alkyl; or R^{7a} and R^{7b}, together with the carbon atom to which they are attached, form an oxo group; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R^{A1} is hydrogen or C₁-C₆-alkyl; each of m and n is independently 1 or 2; and x is 0, 1, or 2.

In some embodiments, A is selected from

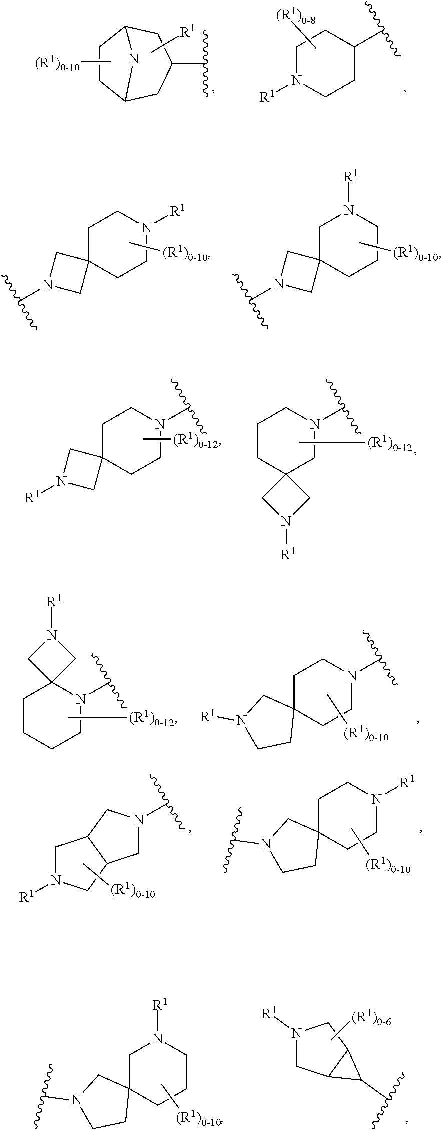

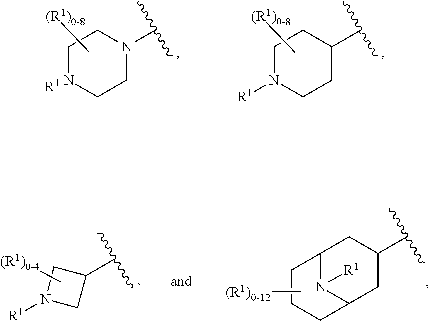

wherein R¹ is as defined herein. In some embodiments, A is selected from

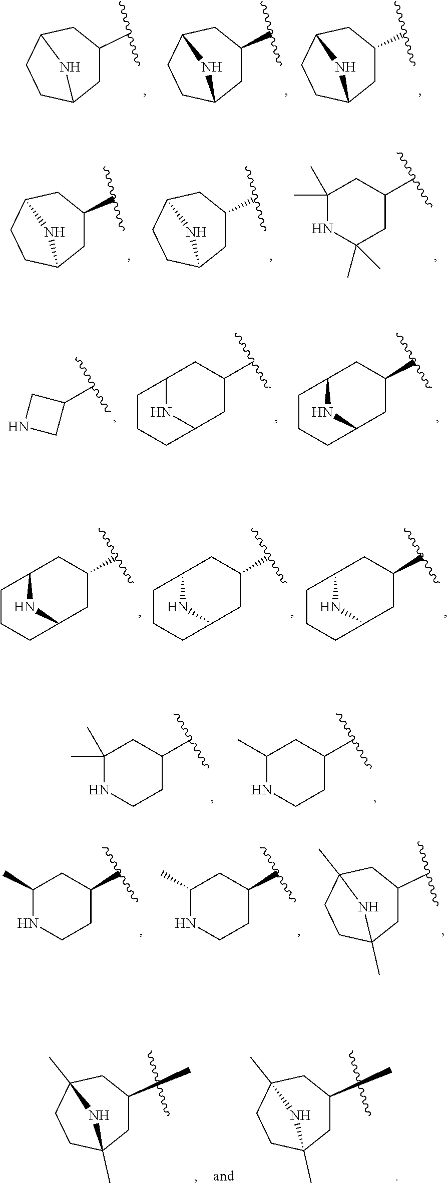

In some embodiments, B is selected from

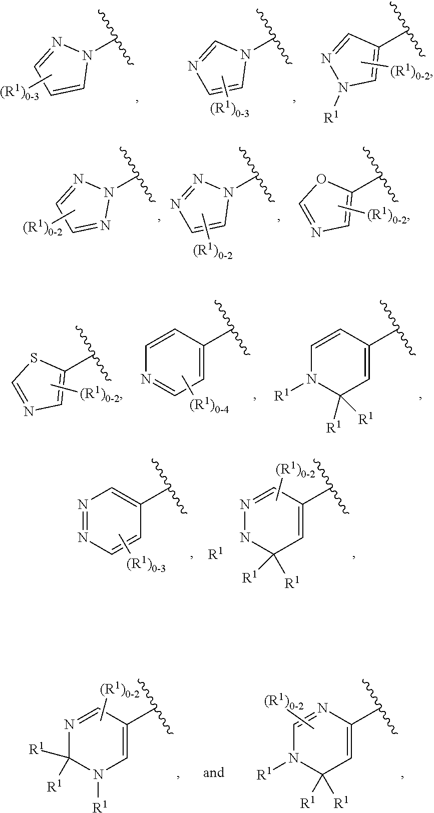

wherein R¹ is as defined herein. In some embodiments, B is selected from

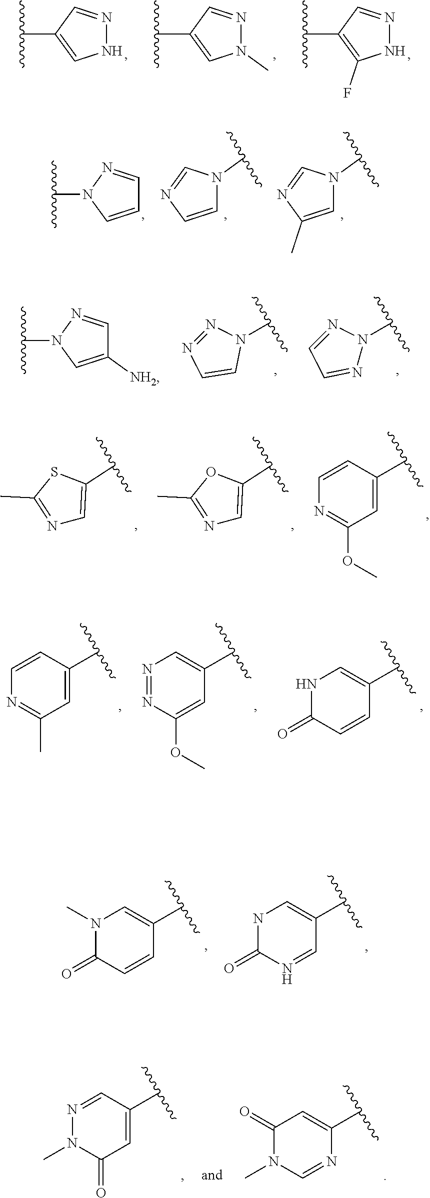

In some embodiments, L is —O—, —S—, or —N(R⁴)—. In some embodiments, L is —O—. In some embodiments, L is —N(R⁴)—. In some embodiments, R⁴ is hydrogen, C₁-C₆ alkyl (e.g., CH₃ or CD₃), or cycloalkyl (e.g., cyclopropyl). In some embodiments, L is —N(CH₃)—. In some embodiments, L is —NH—.

In some embodiments, each of Z¹, Z², Z³, Z⁴, and Z⁵ independently refer to C(R⁶) (e.g., CH) or N. In some embodiments, Z¹ and Z² are each independently C(R⁶), e.g., CH. In some embodiments, Z³ and Z⁴ are each independently C(R⁶), e.g., CH. In some embodiments, one of Z¹ and Z² is C(R⁶), and the other of Z¹ and Z² is N. In some embodiments, one of Z³ and Z⁴ is C(R⁶), and the other of Z³ and Z⁴ is N. In some embodiments, Z¹ is C(R⁶). In some embodiments, Z¹ is N. In some embodiments, Z² is C(R⁶). In some embodiments, Z² is N. In some embodiments, Z³ is C(R⁶). In some embodiments, Z³ is N. In some embodiments, Z⁴ is C(R⁶). In some embodiments, Z⁴ is N. In some embodiments, Z⁵ is C(R⁶). In some embodiments, Z⁵ is N. In some embodiments, Z¹ is C(R⁶) (e.g., CH) and Z² is N. In some embodiments, Z¹ is N and Z² is C(R⁶) (e.g., CH). In some embodiments, Z² and Z⁵ are each independently N.

In some embodiments, one of X and Y is C(R^{7a})(R^{7b}), and the other of X and Y is O. In some embodiments, one of X and Y is C(R^{7a})(R^{7b}), and the other of X and Y is N(R^{7c}). In some embodiments, X is C(R^{7a})(R^{7b}) (e.g., —CH₂—). In some embodiments, X is O. In some embodiments, X is N(R^{7c}). In some embodiments, Y is C(R^{7a})(R^{7b}) (e.g., —CH₂—). In some embodiments, Y is O. In some embodiments, Y is N(R^{7c}). In some embodiments, X is O and Y is C(R^{7a})(R^{7I}) (e.g., —CH₂—). In some embodiments, X is O and Y is —CH₂—. In some embodiments, X is C(R^{7a})(R^{7b}) (e.g., —CH₂—) and Y is O. In some embodiments, X is —CH₂— and Y is O.

In some embodiments, a compound of Formula (I) is a compound of Formula (I-b):

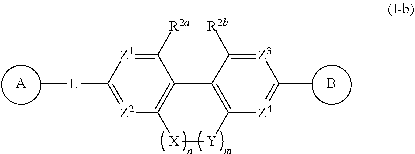

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, and Z⁴ are each independently C(R⁶) or N; X and Y are each independently O, C(R^{7a})(R^{7b}), or N(R^{7c}), wherein X and Y are not both O when n and m are both 1; each of R^{2a} and R^{2b} is independently hydrogen, halo, cyano, C₁-C₆-alkyl, C₂-C₆-alkenyl, C₂-C₆-alkynyl, C₁-C₆-heteroalkyl, C₁-C₆-haloalkyl, or —OR^{A}; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{7a}, R^{7b}, and R^{7c} are each independently hydrogen or C₁-C₆-alkyl; or R^{7a} and R^{7b}, together with the carbon atom to which they are attached, form an oxo group; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R^{A1} is hydrogen or C₁-C₆-alkyl; each of m and n is independently 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl. In some embodiments, A is

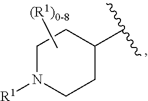

wherein each R¹ is independently hydrogen or C₁-C₆-alkyl. In some embodiments, A is

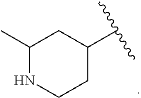 [SMILES: *C1CCNC(C)C1]

In some embodiments, A is

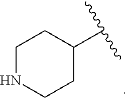 [SMILES: *C1CCNCC1]

In some embodiments, A is

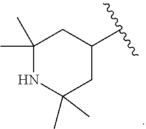 [SMILES: *C1CC(C)(C)NC(C)(C)C1]

In some embodiments, A is

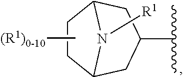

wherein each R¹ is independently hydrogen or C₁-C₆-alkyl. In some embodiments, A is

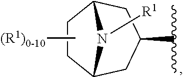

wherein each R¹ is independently hydrogen or C₁-C₆-alkyl. In some embodiments, A is

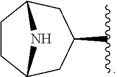 [SMILES: *[C@@H]1C[C@H]2CC[C@@H](C1)N2]

In some embodiments, A is

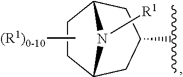

wherein each R¹ is independently hydrogen or C₁-C₆-alkyl. In some embodiments, A is

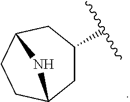 [SMILES: *[C@H]1C[C@H]2CC[C@@H](C1)N2]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, each of Z¹, Z², Z³, and Z⁴ independently refer to C(R⁶) (e.g., CH) or N. In some embodiments, Z¹ and Z² are each independently C(R⁶), e.g., CH. In some embodiments, Z³ and Z⁴ are each independently C(R⁶), e.g., CH. In some embodiments, one of Z¹ and Z² is C(R⁶), and the other of Z¹ and Z² is N. In some embodiments, one of Z³ and Z⁴ is C(R⁶), and the other of Z³ and Z⁴ is N. In some embodiments, Z¹ is C(R⁶). In some embodiments, Z¹ is N. In some embodiments, Z² is C(R⁶). In some embodiments, Z² is N. In some embodiments, Z³ is C(R⁶). In some embodiments, Z³ is N. In some embodiments, Z⁴ is C(R⁶). In some embodiments, Z⁴ is N. In some embodiments, Z¹ is C(R⁶) (e.g., CH) and Z² is N. In some embodiments, Z¹ is N and Z² is C(R⁶) (e.g., CH).

In some embodiments,

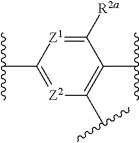

is selected from

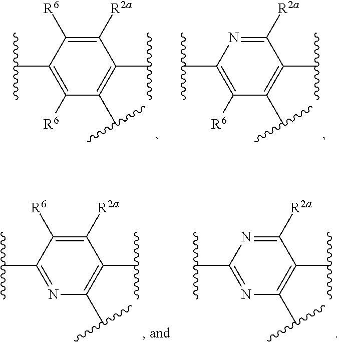

In some embodiments,

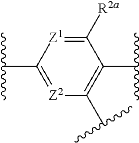

is selected from

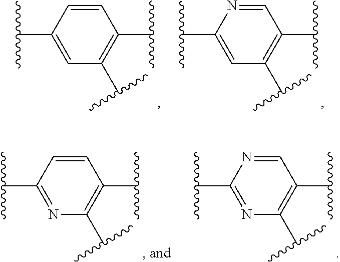

In some embodiments,

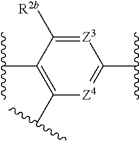

is selected from

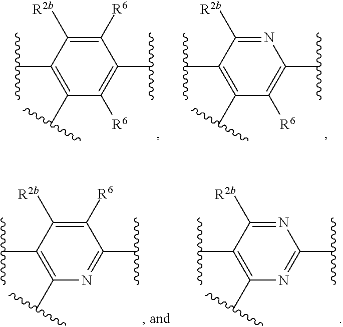

In some embodiments,

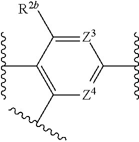

is selected from

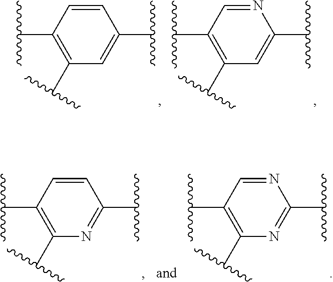

In some embodiments,

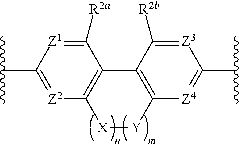

is selected from

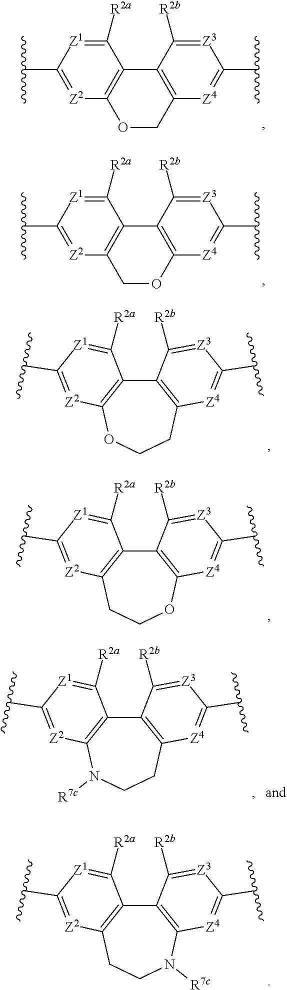

In some embodiments,

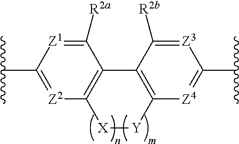

is selected from

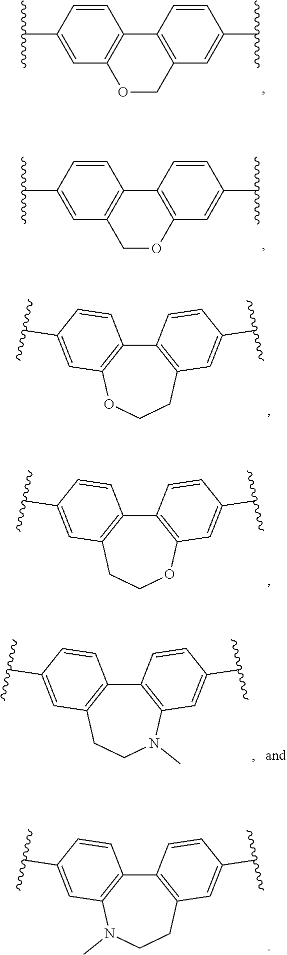 [SMILES: *c1ccc2c(c1)COc1cc(*)ccc1-2 ; *c1ccc2c(c1)COc1cc(*)ccc1-2 ; *c1ccc2c(c1)CCOc1cc(*)ccc1-2 ; *c1ccc2c(c1)CCOc1cc(*)ccc1-2 ; *c1ccc2c(c1)CCN(C)c1cc(*)ccc1-2 ; *c1ccc2c(c1)CCN(C)c1cc(*)ccc1-2]

In some embodiments,

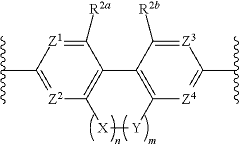

is selected from

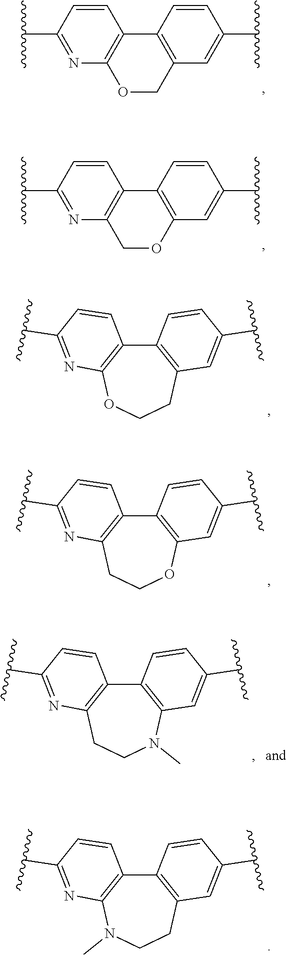 [SMILES: *c1ccc2c(c1)COc1nc(*)ccc1-2 ; *c1ccc2c(c1)OCc1nc(*)ccc1-2 ; *c1ccc2c(c1)CCOc1nc(*)ccc1-2 ; *c1ccc2c(c1)OCCc1nc(*)ccc1-2 ; *c1ccc2c(c1)N(C)CCc1nc(*)ccc1-2 ; *c1ccc2c(c1)CCN(C)c1nc(*)ccc1-2]

In some embodiments, one of X and Y is C(R^{7a})(R^{7b}), and the other of X and Y is O. In some embodiments, one of X and Y is C(R^{7a})(R^{7b}), and the other of X and Y is N(R^{7c}). In some embodiments, X is C(R^{7a})(R^{7b}) (e.g., —CH₂—). In some embodiments, X is O. In some embodiments, X is N(R^{7c}). In some embodiments, Y is C(R^{7a})(R^{7b}) (e.g., —CH₂—). In some embodiments, Y is O. In some embodiments, Y is N(R^{7c}). In some embodiments, X is O and Y is C(R^{7a})(R^{7I}) (e.g., —CH₂—). In some embodiments, X is O and Y is —CH₂—. In some embodiments, X is C(R^{7a})(R^{7b}) (e.g., —CH₂—) and Y is O. In some embodiments, X is —CH₂— and Y is O. In some embodiments, X is N(R^{7c}) (e.g., N(CH₃) and Y is C(R^{7a})(R^{7b}) (e.g., —CH₂—). In some embodiments, X is N(CH₃) and Y is —CH₂—. In some embodiments, Y is N(R^{7c}) (e.g., N(CH₃) and X is C(R^{7a})(R^{7b}) (e.g., —CH₂—). In some embodiments, Y is N(CH₃) and X is —CH₂—.

In some embodiments,

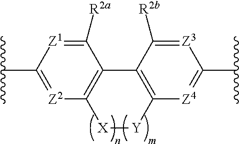

is selected from

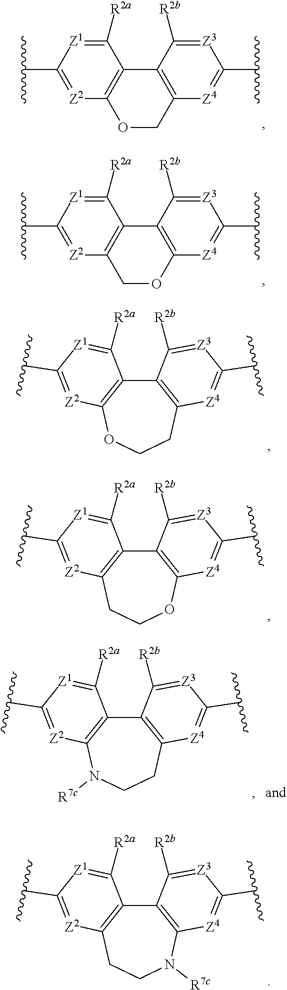

In some embodiments,

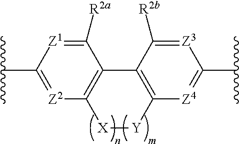

is selected from

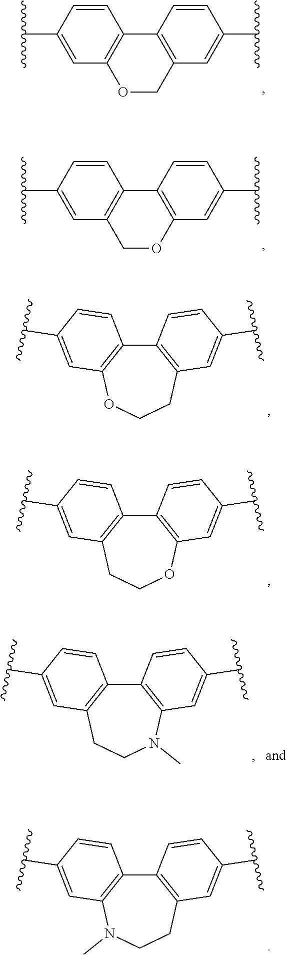 [SMILES: *c1ccc2c(c1)COc1cc(*)ccc1-2 ; *c1ccc2c(c1)COc1cc(*)ccc1-2 ; *c1ccc2c(c1)CCOc1cc(*)ccc1-2 ; *c1ccc2c(c1)CCOc1cc(*)ccc1-2 ; *c1ccc2c(c1)CCN(C)c1cc(*)ccc1-2 ; *c1ccc2c(c1)CCN(C)c1cc(*)ccc1-2]

In some embodiments,

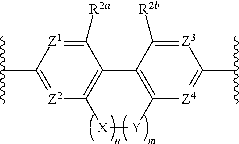

is selected from

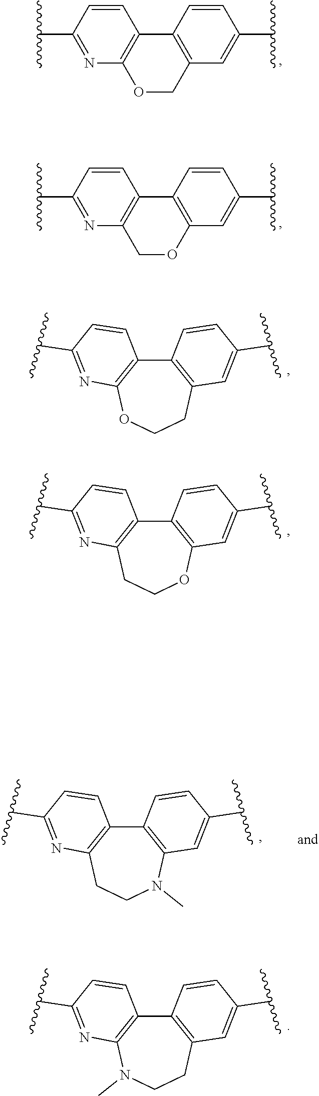 [SMILES: *c1ccc2c(c1)COc1nc(*)ccc1-2 ; *c1ccc2c(c1)OCc1nc(*)ccc1-2 ; *c1ccc2c(c1)CCOc1nc(*)ccc1-2 ; *c1ccc2c(c1)OCCc1nc(*)ccc1-2 ; *c1ccc2c(c1)N(C)CCc1nc(*)ccc1-2 ; *c1ccc2c(c1)CCN(C)c1nc(*)ccc1-2]

In some embodiments, the compound of Formula (I) is a compound of Formula (I-c):

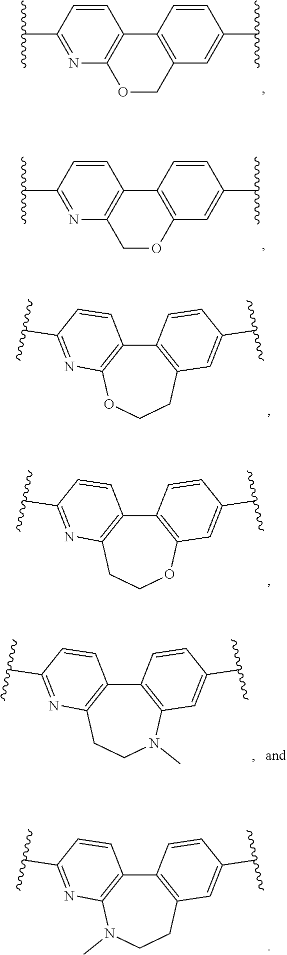 [SMILES: *c1ccc2c(c1)COc1nc(*)ccc1-2 ; *c1ccc2c(c1)OCc1nc(*)ccc1-2 ; *c1ccc2c(c1)CCOc1nc(*)ccc1-2 ; *c1ccc2c(c1)OCCc1nc(*)ccc1-2 ; *c1ccc2c(c1)N(C)CCc1nc(*)ccc1-2 ; *c1ccc2c(c1)CCN(C)c1nc(*)ccc1-2]

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z², Z³, and Z⁴ are each independently C(R⁶) or N; X is O, C(R^{7a})(R^{7b}), or N(R^{7c}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{7a}, R^{7b}, and R^{7c} are each independently hydrogen or C₁-C₆-alkyl; or R^{7a} and R^{7b}, together with the carbon atom to which they are attached, form an oxo group; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R¹⁰; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; m is 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl.

In some embodiments, A is selected from

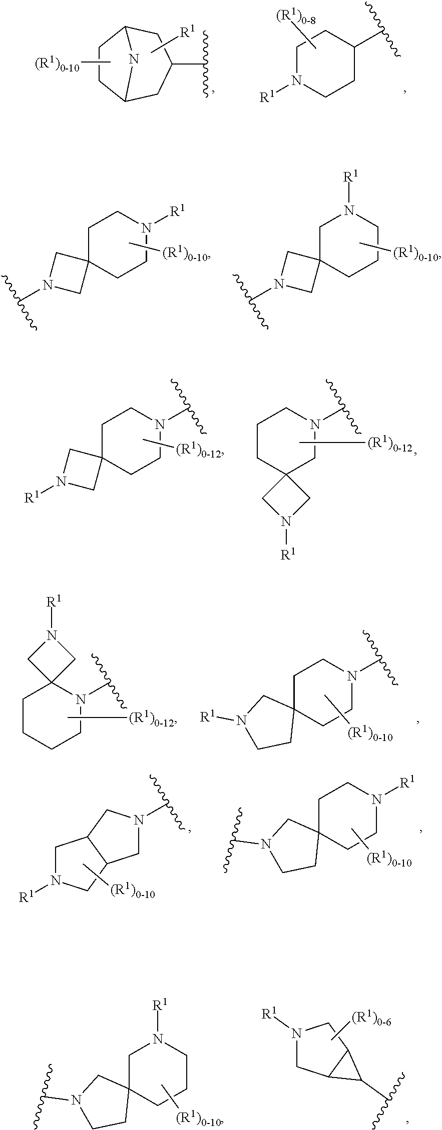

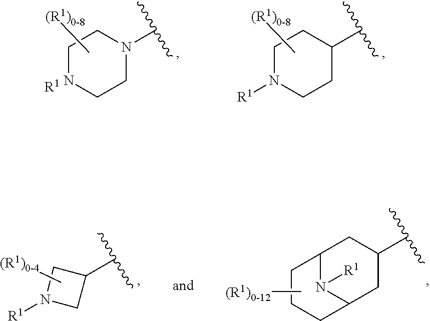

wherein R¹ is as defined herein. In some embodiments, A is selected from

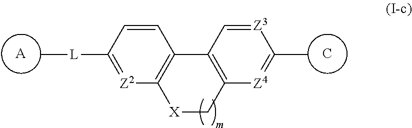

wherein R¹ is as defined herein. In some embodiments, A is selected from

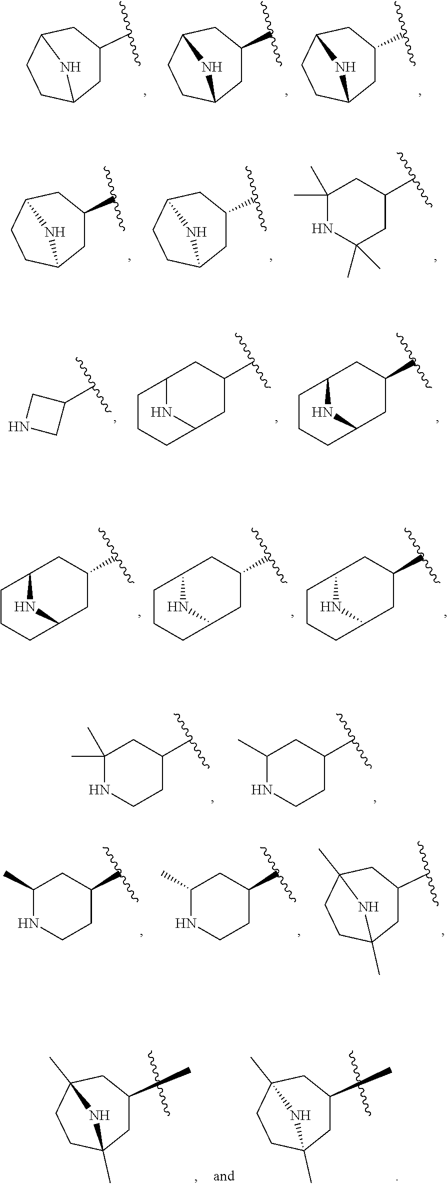

In some embodiments, A is optionally substituted piperidinyl. In some embodiments, A is optionally substituted azabicyclo[3.2.1]octanyl. In some embodiments, A is

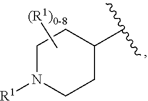

wherein each R¹ is independently hydrogen or C₁-C₆-alkyl. In some embodiments, A is

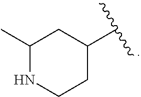 [SMILES: *C1CCNC(C)C1]

In some embodiments, A is

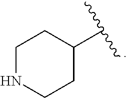 [SMILES: *C1CCNCC1]

In some embodiments, A is

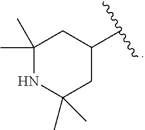 [SMILES: *C1CC(C)(C)NC(C)(C)C1]

In some embodiments, A is

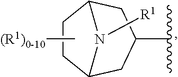

wherein each R¹ is independently hydrogen or C₁-C₆-alkyl. In some embodiments, A is

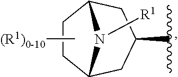

wherein each R¹ is independently hydrogen or C₁-C₆-alkyl. In some embodiments, A is

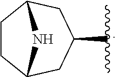 [SMILES: *[C@@H]1C[C@H]2CC[C@@H](C1)N2]

In some embodiments, A is

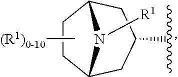

wherein each R¹ is independently hydrogen or C₁-C₆-alkyl. In some embodiments, A is

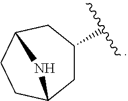 [SMILES: *[C@H]1C[C@H]2CC[C@@H](C1)N2]

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is

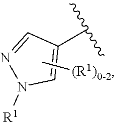

wherein each R¹ is independently hydrogen or C₁-C₆-alkyl. In some embodiments, B is

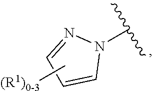

wherein each R¹ is independently hydrogen or C₁-C₆-alkyl. In some embodiments, B is

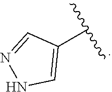 [SMILES: *c1cn[nH]c1]

In some embodiments, B is

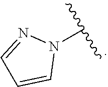 [SMILES: *n1cccn1]

In some embodiments, B is selected from

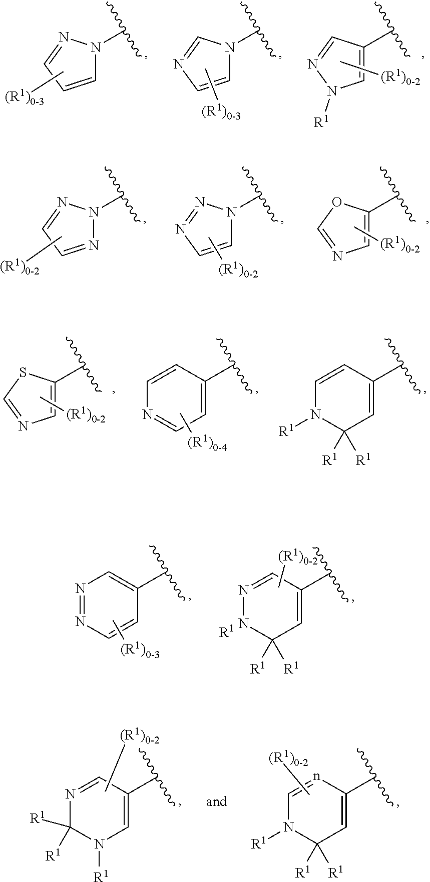

wherein R¹ is as defined herein. In some embodiments, B is selected from

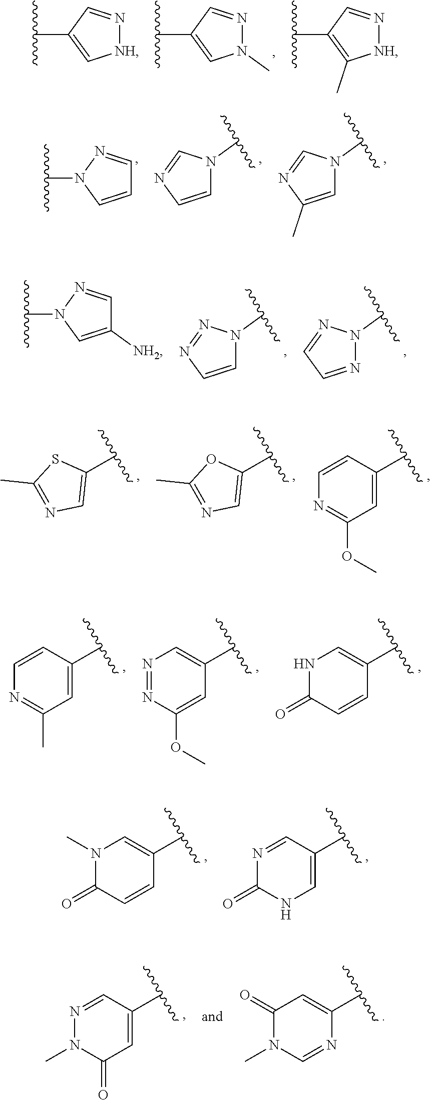

In some embodiments, B is

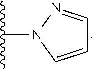 [SMILES: *n1cccn1]

In some embodiments, B is

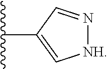 [SMILES: *c1cn[nH]c1]

In some embodiments, B is

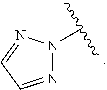 [SMILES: *n1nccn1]

In some embodiments, B is

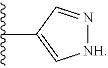 [SMILES: *c1cn[nH]c1]

In some embodiments, B is

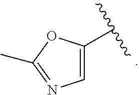 [SMILES: *c1cnc(C)o1]

In some embodiments, B is

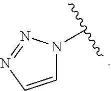 [SMILES: *n1ccnn1]

In some embodiments, B is

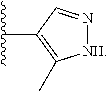 [SMILES: *c1cn[nH]c1C]

In some embodiments, B is

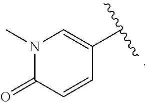 [SMILES: *c1ccc(=O)n(C)c1]

In some embodiments, B is

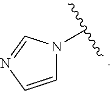 [SMILES: *n1ccnc1]

In some embodiments, B is

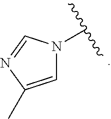 [SMILES: *n1cnc(C)c1]

In some embodiments, B is

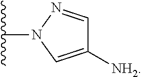 [SMILES: *n1cc(N)cn1]

In some embodiments, B is

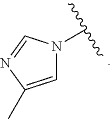 [SMILES: *n1cnc(C)c1]

In some embodiments, B is

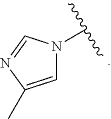 [SMILES: *n1cnc(C)c1]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, each of Z², Z³, and Z⁴, are independently C(R⁶). In some embodiments, Z² is C(R⁶) or N. In some embodiments, Z² is C(R⁶) (e.g., CH). In some embodiments, Z² is N. In some embodiments, Z² is CH. In some embodiments, X is O or C(R^{7a})(R^{7b}). In some embodiments, X is O. In some embodiments, X is O and m is 1. In some embodiments, X is O and m is 2. In some embodiments, X is C(R^{7a})(R^{7b}) (e.g., CH₂). In some embodiments, X is CH₂. In some embodiments, X is N(R^{7c}). In some embodiments, X is N(R^{7c}) and m is 2. In some embodiments, X is N(CH₃). In some embodiments, X is N(CH₃) and m is 2.

In some embodiments, R¹ is hydrogen. In some embodiments, R¹ is C₁-C₆-alkyl (e.g., methyl). In some embodiments, R¹ is methyl. In some embodiments, R⁴ is methyl. In some embodiments R⁶ is hydrogen. In some embodiments, R^{7a} and R^{7b} are each hydrogen. In some embodiments, m is 1. In some embodiments, m is 2.

In some embodiments, the compound of Formula (I) is a compound of Formula (I-d):

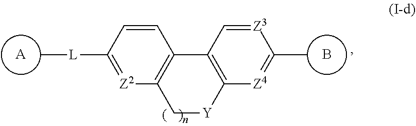

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z², Z³, and Z⁴ are each independently C(R⁶) or N; Y is O, C(R^{7a})(R^{7b}), or N(R^{7c}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{7a}, R^{7b}, and R^{7c} are each independently hydrogen or C₁-C₆-alkyl; or R^{7a} and R^{7b}, together with the carbon atom to which they are attached, form an oxo group; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R¹⁰; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; n is 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl. In some embodiments, A is

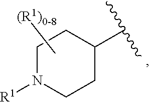

wherein each R¹ is independently hydrogen or C₁-C₆-alkyl. In some embodiments, A is

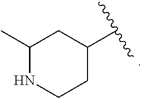 [SMILES: *C1CCNC(C)C1]

In some embodiments, A is

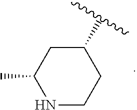 [SMILES: *[C@@H]1CCN[C@H](C)C1]

In some embodiments, A is

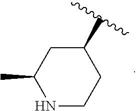 [SMILES: *[C@H]1CCN[C@@H](C)C1]

In some embodiments, A is

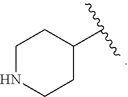 [SMILES: *C1CCNCC1]

In some embodiments, A is

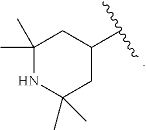 [SMILES: *C1CC(C)(C)NC(C)(C)C1]

In some embodiments, A is

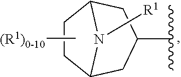

wherein each R¹ is independently hydrogen or C₁-C₆-alkyl. In some embodiments, A is

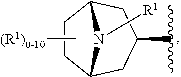

wherein each R¹ is independently hydrogen or C₁-C₆-alkyl. In some embodiments, A is

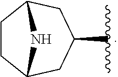 [SMILES: *[C@@H]1C[C@H]2CC[C@@H](C1)N2]

In some embodiments, A is

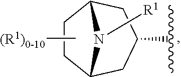

wherein each R¹ is independently hydrogen or C₁-C₆-alkyl. In some embodiments, A is

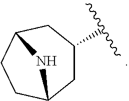 [SMILES: *[C@H]1C[C@H]2CC[C@@H](C1)N2]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, each of Z², Z³, and Z⁴, are independently C(R⁶). In some embodiments, Z² is C(R⁶) or N. In some embodiments, Z² is C(R⁶) (e.g., CH). In some embodiments, Z² is N. In some embodiments, Z² is CH. In some embodiments, Y is O or C(R^{7a})(R^{7b}). In some embodiments, Y is O. In some embodiments, Y is O and n is 1. In some embodiments, Y is O and n is 2. In some embodiments, Y is C(R^{7a})(R^{7b}) (e.g., CH₂). In some embodiments, Y is CH₂. In some embodiments, Y is N(R^{7c}). In some embodiments, Y is N(R^{7c}) and n is 2. In some embodiments, Y is N(CH₃). In some embodiments, Y is N(CH₃) and n is 2.

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is

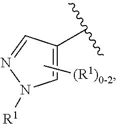

wherein each R¹ is independently hydrogen or C₁-C₆-alkyl. In some embodiments, B is

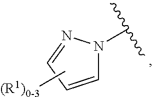

wherein each R¹ is independently hydrogen or C₁-C₆-alkyl. In some embodiments, B is

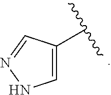 [SMILES: *c1cn[nH]c1]

In some embodiments, B is

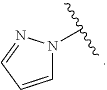 [SMILES: *n1cccn1]

In some embodiments, R¹ is hydrogen. In some embodiments, R¹ is C₁-C₆-alkyl (e.g., methyl). In some embodiments R⁶ is hydrogen. In some embodiments, R^{7a} and R^{7b} are each hydrogen. In some embodiments, n is 1. In some embodiments, n is 2.

In some embodiments, the compound of Formula (I) is a compound of Formula (I-e):

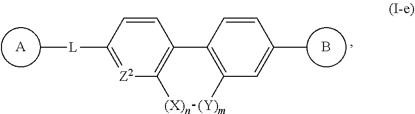

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z² is C(R⁶) or N; X and Y are each independently O, C(R^{7a})(R^{7b}), or N(R^{7c}), wherein X and Y are not both O when n and m are both 1; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{7a}, R^{7b}, and R^{7c} are each independently hydrogen or C₁-C₆-alkyl; or R^{7a} and R^{7b}, together with the carbon atom to which they are attached, form an oxo group; each R⁸ is independently C₁-C₆-alkyl, C₂-C₆-alkenyl, C₂-C₆-alkynyl, C₁-C₆-heteroalkyl, C₁-C₈-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; each of m and n is independently 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl. In some embodiments, A is

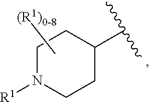

wherein each R¹ is independently hydrogen or C₁-C₆-alkyl. In some embodiments, A is selected from

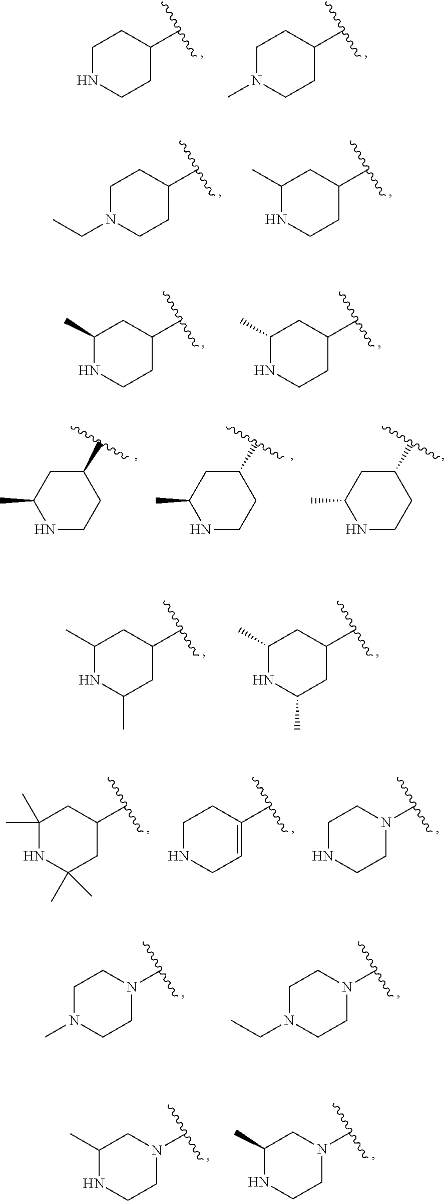

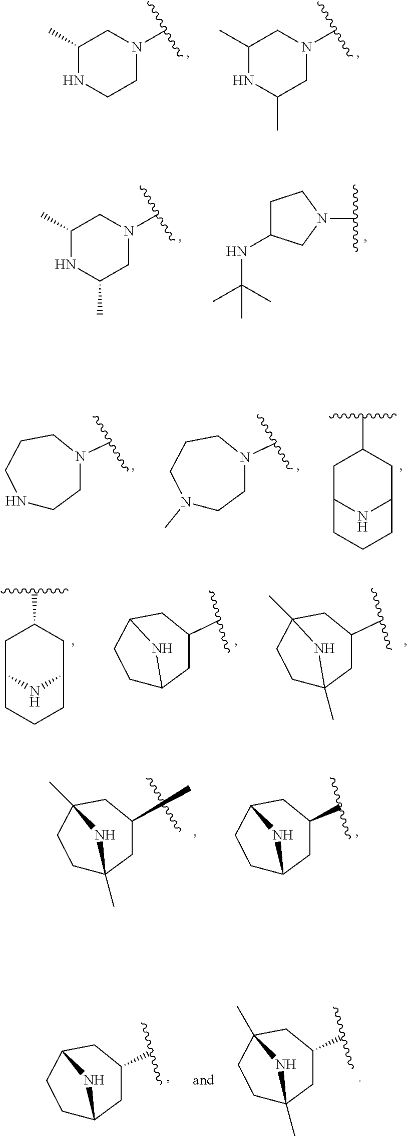

In some embodiments, A is

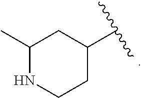 [SMILES: *C1CCNC(C)C1]

In some embodiments, A is

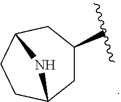 [SMILES: *[C@@H]1C[C@H]2CC[C@@H](C1)N2]

In some embodiments, A is

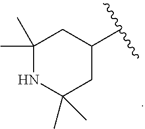 [SMILES: *C1CC(C)(C)NC(C)(C)C1]

in some embodiments, A is

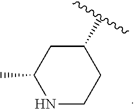 [SMILES: *[C@@H]1CCN[C@H](C)C1]

In some embodiments, A is

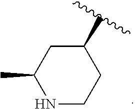 [SMILES: *[C@H]1CCN[C@@H](C)C1]

In some embodiments, A is

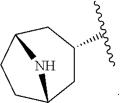 [SMILES: *[C@H]1C[C@H]2CC[C@@H](C1)N2]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, Z² is C(R⁶) or N. In some embodiments, Z² is C(R⁶) (e.g., CH). In some embodiments, Z² is N. In some embodiments, Z² is CH. In some embodiments, X is O or C(R^{7a})(R^{7b}). In some embodiments, X is O. In some embodiments, X is O and m is 1. In some embodiments, X is O and m is 2. In some embodiments, X is C(R^{7a})(R^{7b}) (e.g., CH₂). In some embodiments, X is CH₂. In some embodiments, X is N(R^{7c}). In some embodiments, X is N(R^{7c}) and m is 2. In some embodiments, X is N(CH₃). In some embodiments, X is N(CH₃) and m is 2. In some embodiments, Y is O or C(R^{7a})(R^{7b}). In some embodiments, Y is O. In some embodiments, Y is O and n is 1. In some embodiments, Y is O and n is 2. In some embodiments, Y is C(R^{7a})(R^{7b}) (e.g., CH₂). In some embodiments, Y is CH₂. In some embodiments, Y is N(R^{7c}). In some embodiments, Y is N(R^{7c}) and n is 2. In some embodiments, Y is N(CH₃). In some embodiments, Y is N(CH₃) and n is 2. In some embodiments, X is O and Y is C(R^{7a})(R^{7b}) (e.g., CH₂). In some embodiments, Y is O and X is C(R^{7a})(R^{7b}) (e.g., CH₂). In some embodiments, X is O and Y is CH₂. In some embodiments, Y is O and X is CH₂. In some embodiments, X is N(R^{7c}) and Y is C(R^{7a})(R^{7b}) (e.g., CH₂). In some embodiments, Y is N(R^{7c}) and X is C(R^{7a})(R^{7b}) (e.g., CH₂). In some embodiments, X is N(CH₃) and Y is CH₂. In some embodiments, Y is N(CH₃) and X is CH₂.

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is

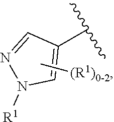

wherein each R¹ is independently hydrogen or C₁-C₆-alkyl. In some embodiments, B is

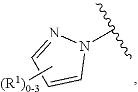

wherein each R¹ is independently hydrogen or C₁-C₆-alkyl. In some embodiments, B is

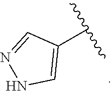 [SMILES: *c1cn[nH]c1]

In some embodiments, B is

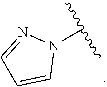 [SMILES: *n1cccn1]

In some embodiments, R¹ is hydrogen. In some embodiments, R¹ is C₁-C₆-alkyl (e.g., methyl). In some embodiments R⁶ is hydrogen. In some embodiments, R^{7a} and R^{7b} are each hydrogen. In some embodiments, n is 1. In some embodiments, n is 2.

In some embodiments, the compound of Formula (I) is a compound of Formula (I-f):

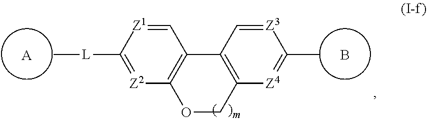

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, and Z⁴ are each independently C(R⁶) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R¹⁰; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl each R¹⁰ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; m is 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl. In some embodiments, A is

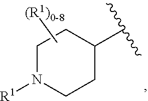

wherein each R¹ is independently hydrogen or C₁-C₆-alkyl.

In some embodiments, A is selected from

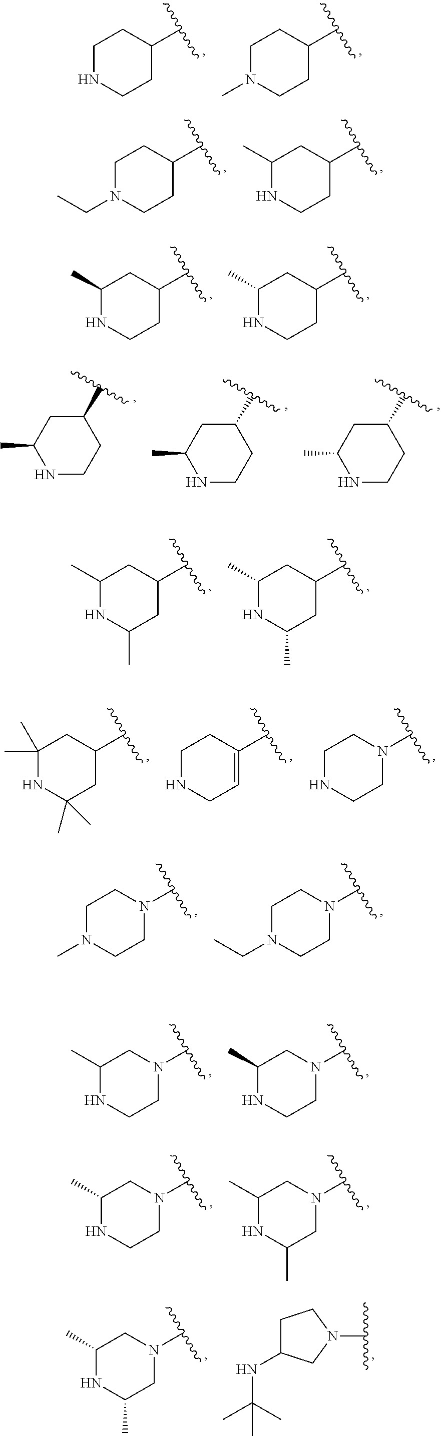

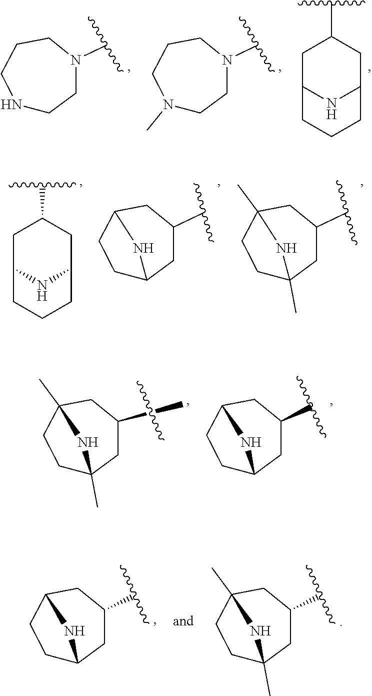

In some embodiments, A is

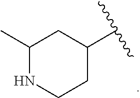 [SMILES: *C1CCNC(C)C1]

In some embodiments, A is

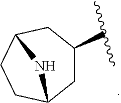 [SMILES: *[C@@H]1C[C@H]2CC[C@@H](C1)N2]

In some embodiments, A is

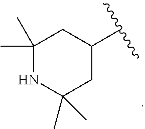 [SMILES: *C1CC(C)(C)NC(C)(C)C1]

In some embodiments, A is

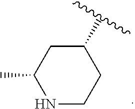 [SMILES: *[C@@H]1CCN[C@H](C)C1]

In some embodiments,

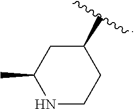 [SMILES: *[C@H]1CCN[C@@H](C)C1]

In some embodiments, A is

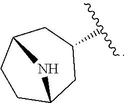 [SMILES: *[C@H]1C[C@H]2CC[C@@H](C1)N2]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, Z¹ is C(R⁶) (e.g., CH). In some embodiments, Z¹ is N. In some embodiments, Z¹ is CH. In some embodiments, Z² is C(R⁶) (e.g., CH). In some embodiments, Z² is N. In some embodiments, Z² is CH. In some embodiments, Z³ is C(R⁶) (e.g., CH). In some embodiments, Z³ is N. In some embodiments, Z³ is CH. In some embodiments, Z⁴ is C(R⁶) (e.g., CH). In some embodiments, Z⁴ is N. In some embodiments, Z⁴ is CH.

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is

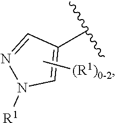

wherein each R¹ is independently hydrogen or C₁-C₆-alkyl. In some embodiments, B is

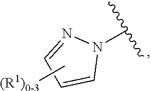

wherein each R¹ is independently hydrogen or C₁-C₆-alkyl. In some embodiments, B is

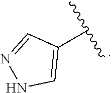 [SMILES: *c1cn[nH]c1]

In some embodiments, B is

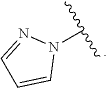 [SMILES: *n1cccn1]

In some embodiments, R¹ is hydrogen. In some embodiments, R¹ is C₁-C₆-alkyl (e.g., methyl). In some embodiments R⁶ is hydrogen. In some embodiments, m is 1. In some embodiments, m is 2.

In some embodiments, the compound of Formula (I) is a compound of Formula (I-g):

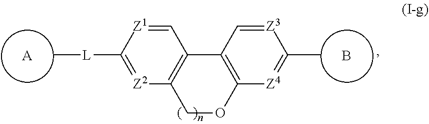

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, and Z⁴ are each independently C(R⁶) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen or C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; m is 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl. In some embodiments, A is

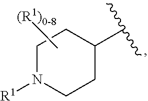

wherein each R¹ is independently hydrogen or C₁-C₆-alkyl.

In some embodiments, A is selected from

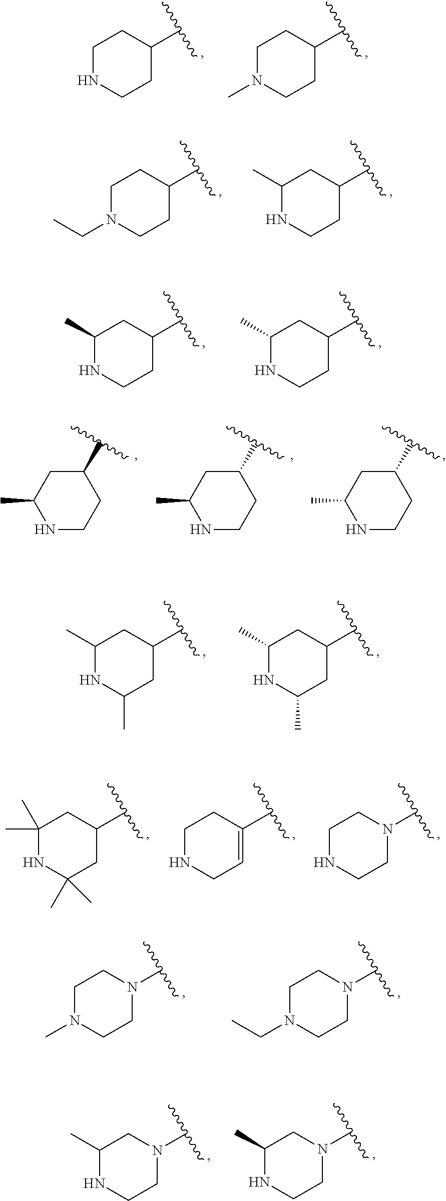

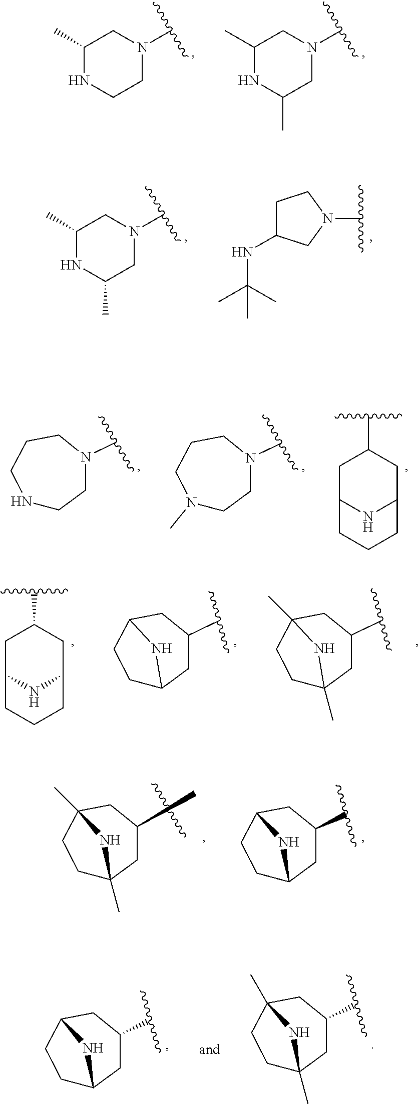

In some embodiments, A is

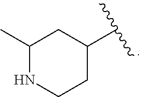 [SMILES: *C1CCNC(C)C1]

In some embodiments, A is

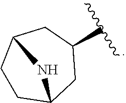 [SMILES: *[C@@H]1C[C@H]2CC[C@@H](C1)N2]

In some embodiments, A is

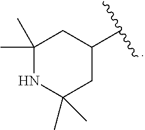 [SMILES: *C1CC(C)(C)NC(C)(C)C1]

In some embodiments, A is

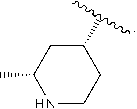 [SMILES: *[C@@H]1CCN[C@H](C)C1]

In some embodiments, A is

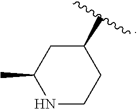 [SMILES: *[C@H]1CCN[C@@H](C)C1]

In some embodiments, A is

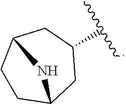 [SMILES: *[C@H]1C[C@H]2CC[C@@H](C1)N2]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, Z¹ is C(R⁶) (e.g., CH). In some embodiments, Z¹ is N. In some embodiments, Z¹ is CH. In some embodiments, Z² is C(R⁶) (e.g., CH). In some embodiments, Z² is N. In some embodiments, Z² is CH. In some embodiments, Z³ is C(R⁶) (e.g., CH). In some embodiments, Z³ is N. In some embodiments, Z³ is CH. In some embodiments, Z⁴ is C(R⁶) (e.g., CH). In some embodiments, Z⁴ is N. In some embodiments, Z⁴ is CH.

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is

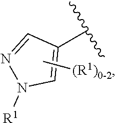

wherein each R¹ is independently hydrogen or C₁-C₆-alkyl. In some embodiments, B is

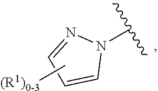

wherein each R¹ is independently hydrogen or C₁-C₆-alkyl. In some embodiments, B is

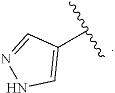 [SMILES: *c1cn[nH]c1]

In some embodiments, B is

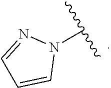 [SMILES: *n1cccn1]

In some embodiments, R¹ is hydrogen. In some embodiments, R¹ is C₁-C₆-alkyl (e.g., methyl). In some embodiments R⁶ is hydrogen. In some embodiments, n is 1. In some embodiments, n is 2.

In some embodiments, the compound of Formula (I) is a compound of Formula (I-h):

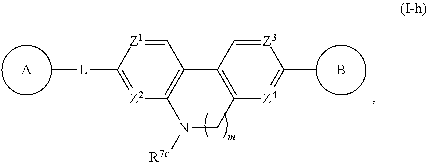

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; Z¹, Z², Z³, and Z⁴ are each independently C(R⁶) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁶ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{7c} is hydrogen or C₁-C₆-alkyl; each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen or C₁-C₆ alkyl, C₁-C₆ heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R¹⁰ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; m is 1 or 2; and x is 0, 1, or 2.

In some embodiments, A is heterocyclyl optionally substituted with one or more R¹. In some embodiments, A is bicyclic heterocyclyl. In some embodiments, A is monocyclic nitrogen-containing heterocyclyl. In some embodiments, A is bicyclic nitrogen-containing heterocyclyl. In some embodiments, A is optionally substituted piperidinyl. In some embodiments, A is optionally substituted azabicyclo[3.2.1]octanyl. In some embodiments, A is

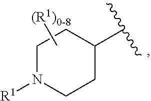

wherein each R¹ is independently hydrogen or C₁-C₆-alkyl.

In some embodiments, A is selected from

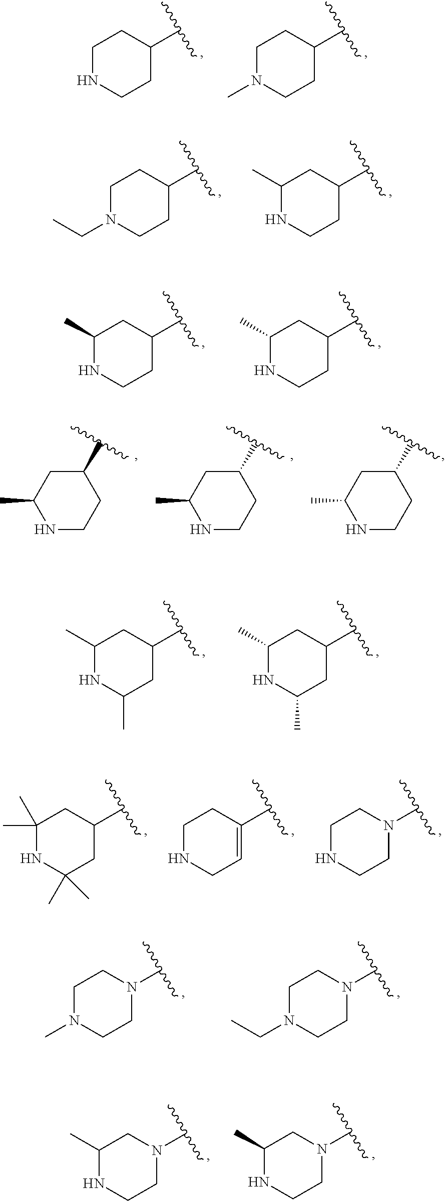

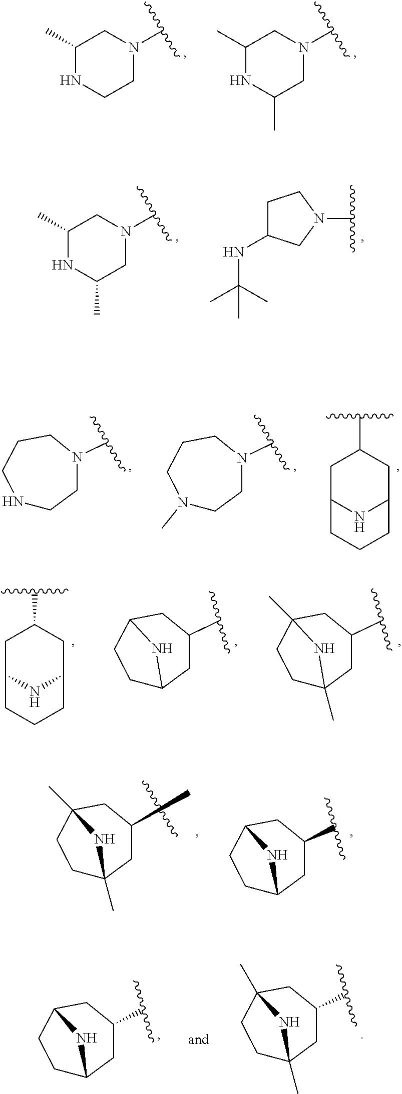

In some embodiments, A is

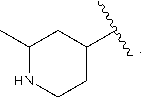 [SMILES: *C1CCNC(C)C1]

In some embodiments, A is

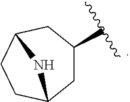 [SMILES: *[C@@H]1C[C@H]2CC[C@@H](C1)N2]

In some embodiments, A is

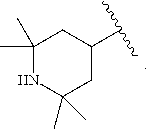 [SMILES: *C1CC(C)(C)NC(C)(C)C1]

In some embodiments, A is

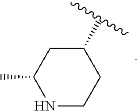 [SMILES: *[C@@H]1CCN[C@H](C)C1]

In some embodiments, A is

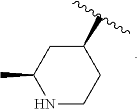 [SMILES: *[C@H]1CCN[C@@H](C)C1]

In some embodiments, A is

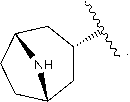 [SMILES: *[C@H]1C[C@H]2CC[C@@H](C1)N2]

In some embodiments, L is oxygen. In some embodiments, L is nitrogen that is optionally substituted with R⁴. In some embodiments, L is nitrogen substituted with one R⁴. In some embodiments, L is —N(CH₃)—. In some embodiments, L is —NH—.

In some embodiments, Z¹ is C(R⁶) (e.g., CH). In some embodiments, Z¹ is N. In some embodiments, Z¹ is CH. In some embodiments, Z² is C(R⁶) (e.g., CH). In some embodiments, Z² is N. In some embodiments, Z² is CH. In some embodiments, Z³ is C(R⁶) (e.g., CH). In some embodiments, Z³ is N. In some embodiments, Z³ is CH. In some embodiments, Z⁴ is C(R⁶) (e.g., CH). In some embodiments, Z⁴ is N. In some embodiments, Z⁴ is CH.

In some embodiments, B is heteroaryl optionally substituted with one or more R¹. In some embodiments, B is monocyclic heteroaryl. In some embodiments, B is monocyclic nitrogen-containing heteroaryl. In some embodiments, B is optionally substituted pyrazolyl. In some embodiments, B is

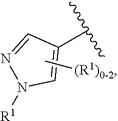

wherein each R¹ is independently hydrogen or C₁-C₆-alkyl. In some embodiments, B is

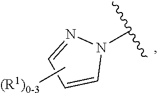

wherein each R¹ is independently hydrogen or C₁-C₆-alkyl. In some embodiments, B is

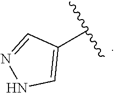 [SMILES: *c1cn[nH]c1]

In some embodiments, B is

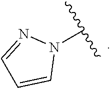 [SMILES: *n1cccn1]

In some embodiments, R¹ is hydrogen. In some embodiments, R¹ is C₁-C₆-alkyl (e.g., methyl). In some embodiments R⁶ is hydrogen. In some embodiments R⁷, is hydrogen. In some embodiments R⁷, is C₁-C₆-alkyl (e.g., methyl). In some embodiments R⁷, is methyl. In some embodiments, m is 1. In some embodiments, m is 2.

In some embodiments, the compound of Formula (I) is a compound selected from a compound in Table 1, or a pharmaceutically acceptable salt, solvate, hydrate, tautomer, or stereoisomer thereof.

TABLE 1
Exemplary compounds of Formula (I)

| Compound No. | Structure |
|---|---|
| 100 | 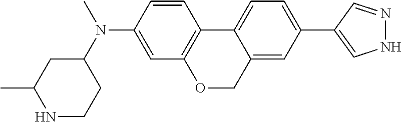 [SMILES: CC1CC(N(C)c2ccc3c(c2)OCc2cc(-c4cn[nH]c4)ccc2-3)CCN1] |
| 101 | 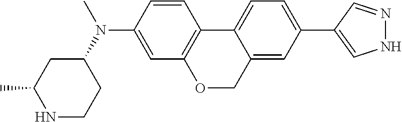 [SMILES: C[C@@H]1C[C@H](N(C)c2ccc3c(c2)OCc2cc(-c4cn[nH]c4)ccc2-3)CCN1] |
| 102 | 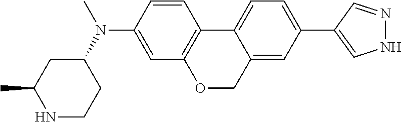 [SMILES: C[C@H]1C[C@H](N(C)c2ccc3c(c2)OCc2cc(-c4cn[nH]c4)ccc2-3)CCN1] |
| 103 | 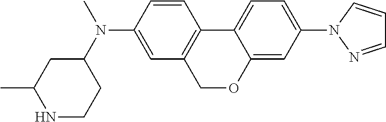 [SMILES: CC1CC(N(C)c2ccc3c(c2)COc2cc(-n4cccn4)ccc2-3)CCN1] |
| 104 | 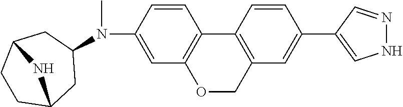 [SMILES: CN(c1ccc2c(c1)OCc1cc(-c3cn[nH]c3)ccc1-2)[C@@H]1C[C@H]2CC[C@@H](C1)N2] |
| 105 | 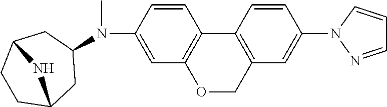 [SMILES: CN(c1ccc2c(c1)OCc1cc(-n3cccn3)ccc1-2)[C@@H]1C[C@H]2CC[C@@H](C1)N2] |
| 106 | 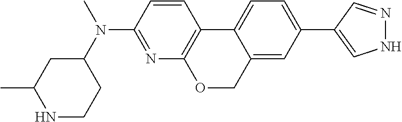 [SMILES: CC1CC(N(C)c2ccc3c(n2)OCc2cc(-c4cn[nH]c4)ccc2-3)CCN1] |
| 107 | 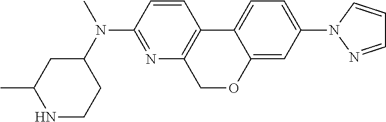 [SMILES: CC1CC(N(C)c2ccc3c(n2)COc2cc(-n4cccn4)ccc2-3)CCN1] |
| 108 | 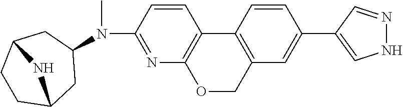 [SMILES: CN(c1ccc2c(n1)OCc1cc(-c3cn[nH]c3)ccc1-2)[C@@H]1C[C@H]2CC[C@@H](C1)N2] |
| 109 | 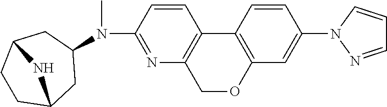 [SMILES: CN(c1ccc2c(n1)COc1cc(-n3cccn3)ccc1-2)[C@@H]1C[C@H]2CC[C@@H](C1)N2] |
| 110 | 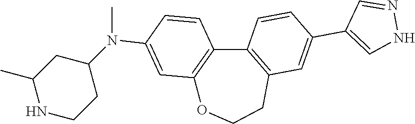 [SMILES: CC1CC(N(C)c2ccc3c(c2)OCCc2cc(-c4cn[nH]c4)ccc2-3)CCN1] |
| 111 | 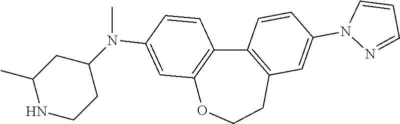 [SMILES: CC1CC(N(C)c2ccc3c(c2)OCCc2cc(-n4cccn4)ccc2-3)CCN1] |
| 112 | 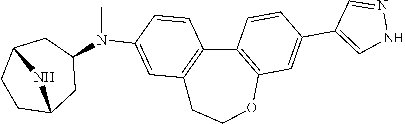 [SMILES: CN(c1ccc2c(c1)CCOc1cc(-c3cn[nH]c3)ccc1-2)[C@@H]1C[C@H]2CC[C@@H](C1)N2] |
| 113 | 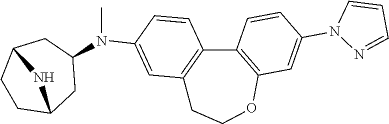 [SMILES: CN(c1ccc2c(c1)CCOc1cc(-n3cccn3)ccc1-2)[C@@H]1C[C@H]2CC[C@@H](C1)N2] |
| 114 | 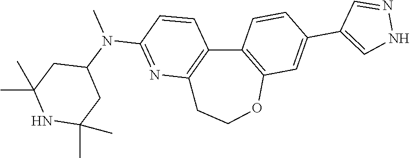 [SMILES: CN(c1ccc2c(n1)CCOc1cc(-c3cn[nH]c3)ccc1-2)C1CC(C)(C)NC(C)(C)C1] |
| 115 | 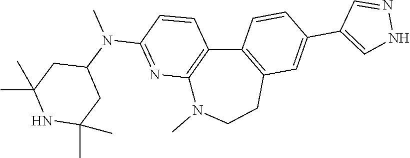 [SMILES: CN1CCc2cc(-c3cn[nH]c3)ccc2-c2ccc(N(C)C3CC(C)(C)NC(C)(C)C3)nc21] |
| 116 | 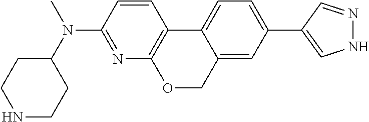 [SMILES: CN(c1ccc2c(n1)OCc1cc(-c3cn[nH]c3)ccc1-2)C1CCNCC1] |
| 117 | 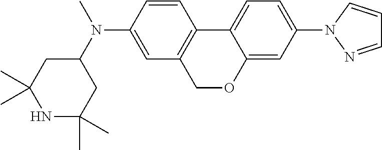 [SMILES: CN(c1ccc2c(c1)COc1cc(-n3cccn3)ccc1-2)C1CC(C)(C)NC(C)(C)C1] |
| 118 | 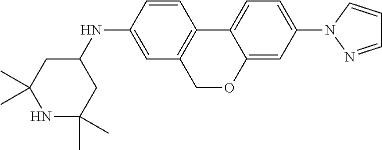 [SMILES: CC1(C)CC(Nc2ccc3c(c2)COc2cc(-n4cccn4)ccc2-3)CC(C)(C)N1] |
| 119 | 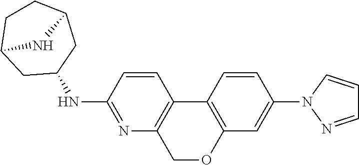 [SMILES: c1cnn(-c2ccc3c(c2)OCc2nc(N[C@@H]4C[C@H]5CC[C@@H](C4)N5)ccc2-3)c1] |
| 121 | 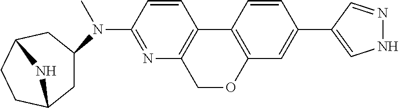 [SMILES: CN(c1ccc2c(n1)COc1cc(-c3cn[nH]c3)ccc1-2)[C@@H]1C[C@H]2CC[C@@H](C1)N2] |
| 122 | 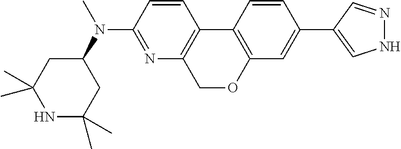 |
| 123 | 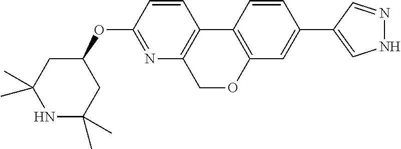 |
| 124 | 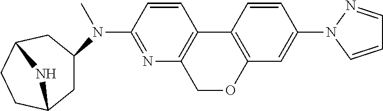 [SMILES: CN(c1ccc2c(n1)COc1cc(-n3cccn3)ccc1-2)[C@@H]1C[C@H]2CC[C@@H](C1)N2] |
| 125 | 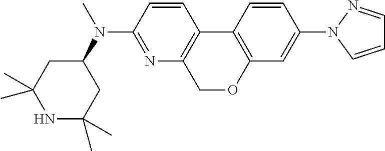 |
| 127 | 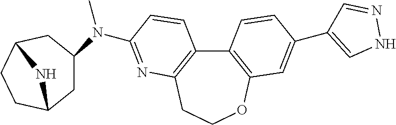 [SMILES: CN(c1ccc2c(n1)CCOc1cc(-c3cn[nH]c3)ccc1-2)[C@@H]1C[C@H]2CC[C@@H](C1)N2] |
| 128 | 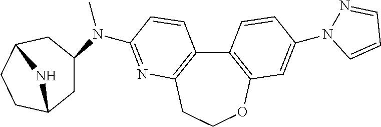 [SMILES: CN(c1ccc2c(n1)CCOc1cc(-n3cccn3)ccc1-2)[C@@H]1C[C@H]2CC[C@@H](C1)N2] |
| 129 | 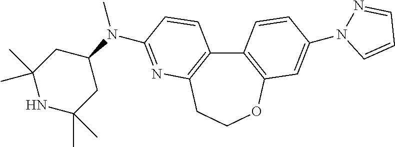 |
| 130 | 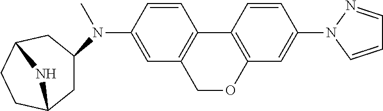 [SMILES: CN(c1ccc2c(c1)COc1cc(-n3cccn3)ccc1-2)[C@@H]1C[C@H]2CC[C@@H](C1)N2] |
| 131 | 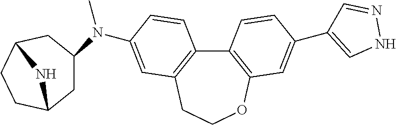 [SMILES: CN(c1ccc2c(c1)CCOc1cc(-c3cn[nH]c3)ccc1-2)[C@@H]1C[C@H]2CC[C@@H](C1)N2] |
| 132 | 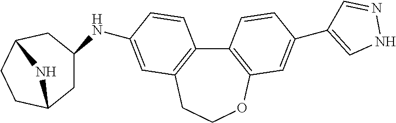 [SMILES: c1cc2c(cc1N[C@@H]1C[C@H]3CC[C@@H](C1)N3)CCOc1cc(-c3cn[nH]c3)ccc1-2] |
| 134 | 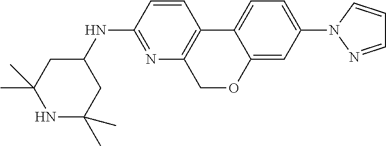 [SMILES: CC1(C)CC(Nc2ccc3c(n2)COc2cc(-n4cccn4)ccc2-3)CC(C)(C)N1] |
| 135 | 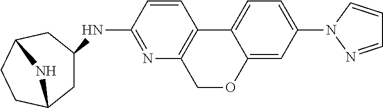 [SMILES: c1cnn(-c2ccc3c(c2)OCc2nc(N[C@@H]4C[C@H]5CC[C@@H](C4)N5)ccc2-3)c1] |
| 136 | 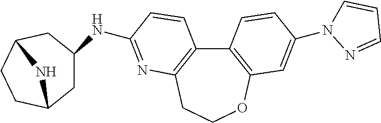 [SMILES: c1cnn(-c2ccc3c(c2)OCCc2nc(N[C@@H]4C[C@H]5CC[C@@H](C4)N5)ccc2-3)c1] |
| 137 | 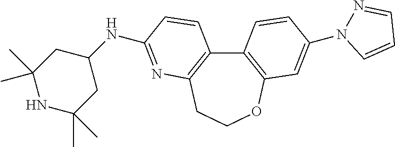 [SMILES: CC1(C)CC(Nc2ccc3c(n2)CCOc2cc(-n4cccn4)ccc2-3)CC(C)(C)N1] |
| 138 | 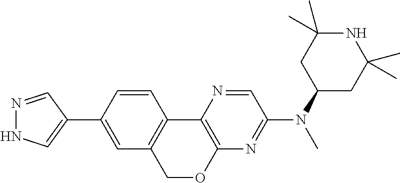 |
| 139 | 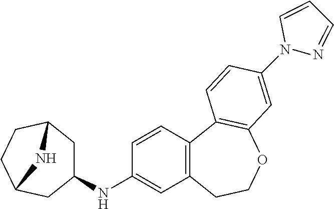 [SMILES: c1cnn(-c2ccc3c(c2)OCCc2cc(N[C@@H]4C[C@H]5CC[C@@H](C4)N5)ccc2-3)c1] |
| 140 | 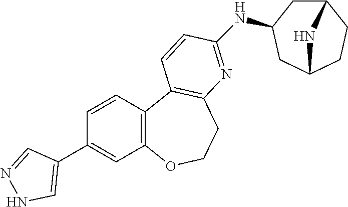 [SMILES: c1cc2c(cc1-c1cn[nH]c1)OCCc1nc(N[C@@H]3C[C@H]4CC[C@@H](C3)N4)ccc1-2] |
| 141 | 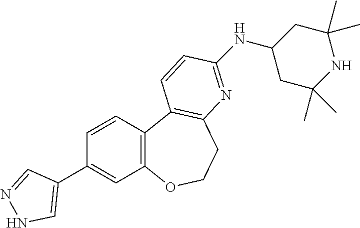 [SMILES: CC1(C)CC(Nc2ccc3c(n2)CCOc2cc(-c4cn[nH]c4)ccc2-3)CC(C)(C)N1] |
| 142 | 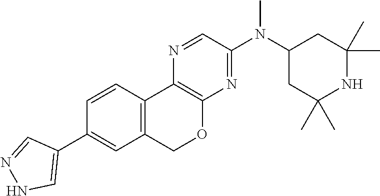 [SMILES: CN(c1cnc2c(n1)OCc1cc(-c3cn[nH]c3)ccc1-2)C1CC(C)(C)NC(C)(C)C1] |
| 143 | 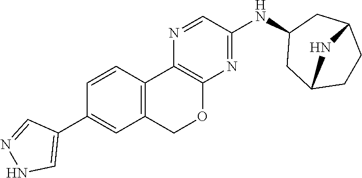 [SMILES: c1cc2c(cc1-c1cn[nH]c1)COc1nc(N[C@@H]3C[C@H]4CC[C@@H](C3)N4)cnc1-2] |
| 144 | 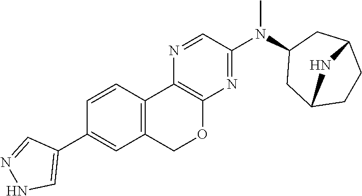 [SMILES: CN(c1cnc2c(n1)OCc1cc(-c3cn[nH]c3)ccc1-2)[C@@H]1C[C@H]2CC[C@@H](C1)N2] |
| 145 | 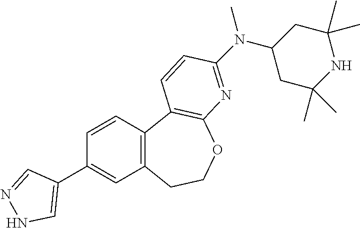 [SMILES: CN(c1ccc2c(n1)OCCc1cc(-c3cn[nH]c3)ccc1-2)C1CC(C)(C)NC(C)(C)C1] |
| 146 | 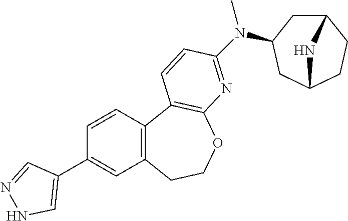 [SMILES: CN(c1ccc2c(n1)OCCc1cc(-c3cn[nH]c3)ccc1-2)[C@@H]1C[C@H]2CC[C@@H](C1)N2] |
| 147 | 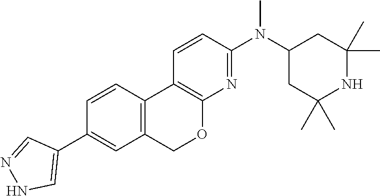 [SMILES: CN(c1ccc2c(n1)OCc1cc(-c3cn[nH]c3)ccc1-2)C1CC(C)(C)NC(C)(C)C1] |
| 148 | 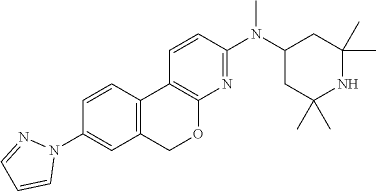 [SMILES: CN(c1ccc2c(n1)OCc1cc(-n3cccn3)ccc1-2)C1CC(C)(C)NC(C)(C)C1] |
| 149 | 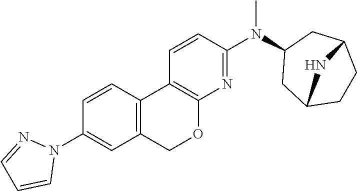 [SMILES: CN(c1ccc2c(n1)OCc1cc(-n3cccn3)ccc1-2)[C@@H]1C[C@H]2CC[C@@H](C1)N2] |
| 150 | 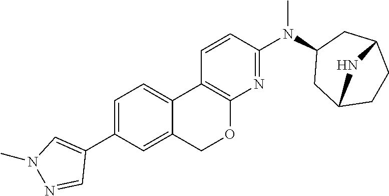 [SMILES: CN(c1ccc2c(n1)OCc1cc(-c3cnn(C)c3)ccc1-2)[C@@H]1C[C@H]2CC[C@@H](C1)N2] |
| 151 | 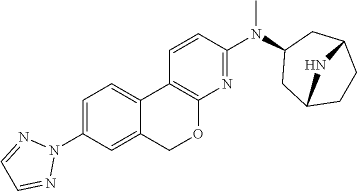 [SMILES: CN(c1ccc2c(n1)OCc1cc(-n3nccn3)ccc1-2)[C@@H]1C[C@H]2CC[C@@H](C1)N2] |
| 152 | 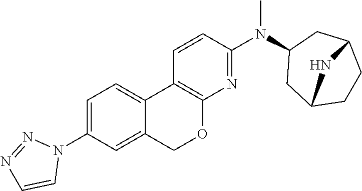 [SMILES: CN(c1ccc2c(n1)OCc1cc(-n3ccnn3)ccc1-2)[C@@H]1C[C@H]2CC[C@@H](C1)N2] |
| 153 | 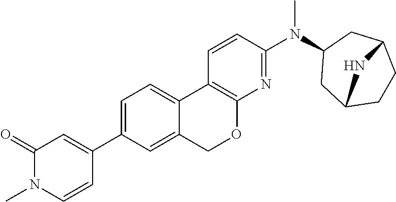 [SMILES: CN(c1ccc2c(n1)OCc1cc(-c3ccn(C)c(=O)c3)ccc1-2)[C@@H]1C[C@H]2CC[C@@H](C1)N2] |
| 154 | 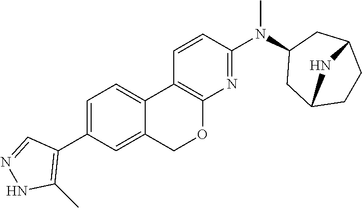 [SMILES: Cc1[nH]ncc1-c1ccc2c(c1)COc1nc(N(C)[C@@H]3C[C@H]4CC[C@@H](C3)N4)ccc1-2] |
| 155 | 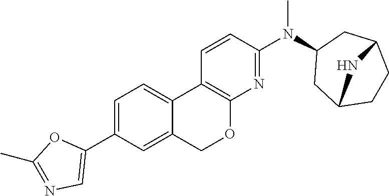 [SMILES: Cc1ncc(-c2ccc3c(c2)COc2nc(N(C)[C@@H]4C[C@H]5CC[C@@H](C4)N5)ccc2-3)o1] |
| 156 | 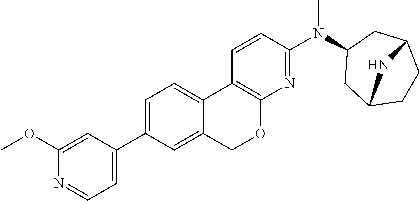 [SMILES: COc1cc(-c2ccc3c(c2)COc2nc(N(C)[C@@H]4C[C@H]5CC[C@@H](C4)N5)ccc2-3)ccn1] |
| 157 | 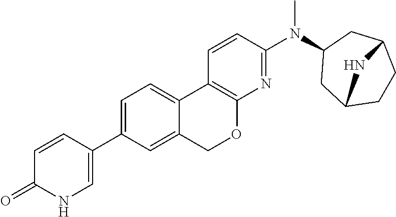 [SMILES: CN(c1ccc2c(n1)OCc1cc(-c3ccc(=O)[nH]c3)ccc1-2)[C@@H]1C[C@H]2CC[C@@H](C1)N2] |
| 158 | 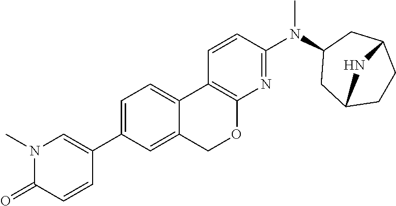 [SMILES: CN(c1ccc2c(n1)OCc1cc(-c3ccc(=O)n(C)c3)ccc1-2)[C@@H]1C[C@H]2CC[C@@H](C1)N2] |
| 159 | 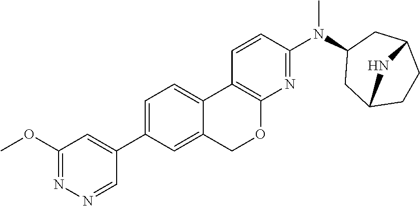 [SMILES: COc1cc(-c2ccc3c(c2)COc2nc(N(C)[C@@H]4C[C@H]5CC[C@@H](C4)N5)ccc2-3)cnn1] |
| 160 | 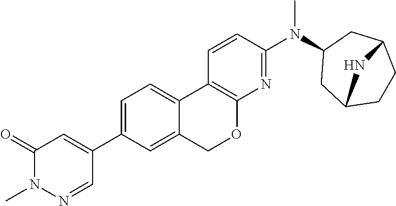 [SMILES: CN(c1ccc2c(n1)OCc1cc(-c3cnn(C)c(=O)c3)ccc1-2)[C@@H]1C[C@H]2CC[C@@H](C1)N2] |
| 161 | 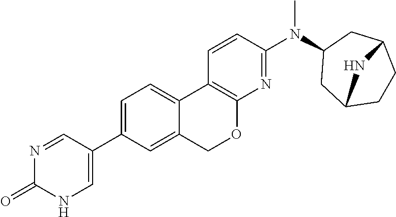 [SMILES: CN(c1ccc2c(n1)OCc1cc(-c3cnc(=O)[nH]c3)ccc1-2)[C@@H]1C[C@H]2CC[C@@H](C1)N2] |
| 162 | 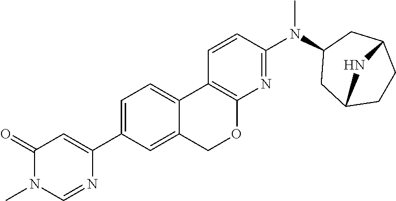 [SMILES: CN(c1ccc2c(n1)OCc1cc(-c3cc(=O)n(C)cn3)ccc1-2)[C@@H]1C[C@H]2CC[C@@H](C1)N2] |
| 163 | 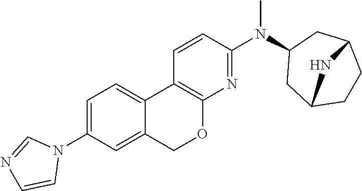 [SMILES: CN(c1ccc2c(n1)OCc1cc(-n3ccnc3)ccc1-2)[C@@H]1C[C@H]2CC[C@@H](C1)N2] |
| 164 | 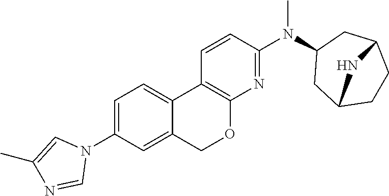 [SMILES: Cc1cn(-c2ccc3c(c2)COc2nc(N(C)[C@@H]4C[C@H]5CC[C@@H](C4)N5)ccc2-3)cn1] |
| 165 | 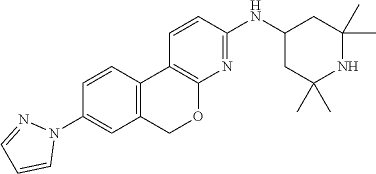 [SMILES: CC1(C)CC(Nc2ccc3c(n2)OCc2cc(-n4cccn4)ccc2-3)CC(C)(C)N1] |
| 166 | 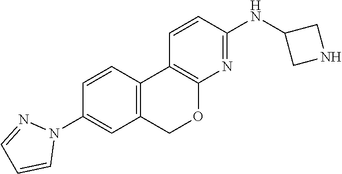 [SMILES: c1cnn(-c2ccc3c(c2)COc2nc(NC4CNC4)ccc2-3)c1] |
| 167 | 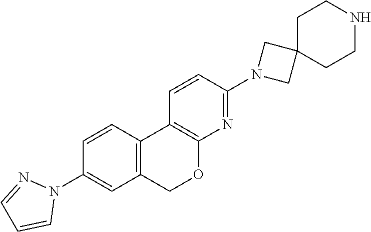 [SMILES: c1cnn(-c2ccc3c(c2)COc2nc(N4CC5(CCNCC5)C4)ccc2-3)c1] |
| 168 | 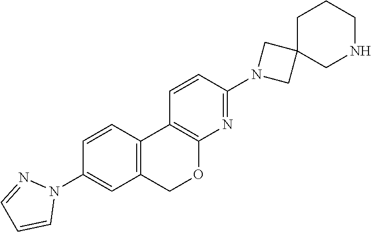 [SMILES: c1cnn(-c2ccc3c(c2)COc2nc(N4CC5(CCCNC5)C4)ccc2-3)c1] |
| 169 | 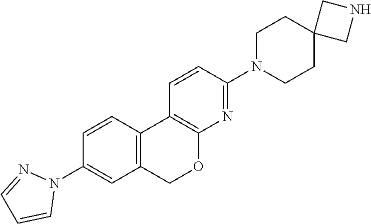 [SMILES: c1cnn(-c2ccc3c(c2)COc2nc(N4CCC5(CC4)CNC5)ccc2-3)c1] |
| 170 | 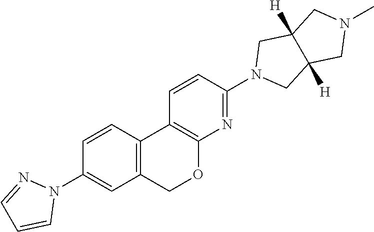 [SMILES: CN1C[C@@H]2CN(c3ccc4c(n3)OCc3cc(-n5cccn5)ccc3-4)C[C@@H]2C1] |
| 171 | 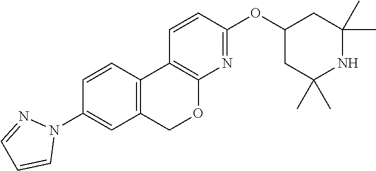 [SMILES: CC1(C)CC(Oc2ccc3c(n2)OCc2cc(-n4cccn4)ccc2-3)CC(C)(C)N1] |
| 172 | 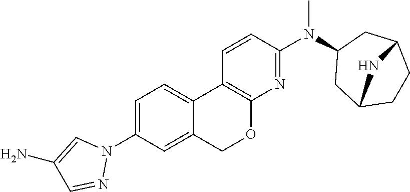 [SMILES: CN(c1ccc2c(n1)OCc1cc(-n3cc(N)cn3)ccc1-2)[C@@H]1C[C@H]2CC[C@@H](C1)N2] |
| 173 | 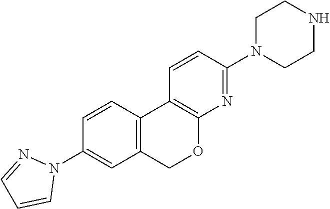 [SMILES: c1cnn(-c2ccc3c(c2)COc2nc(N4CCNCC4)ccc2-3)c1] |
| 174 | 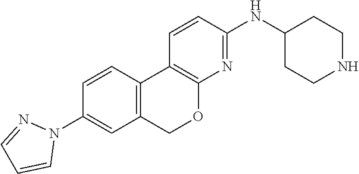 [SMILES: c1cnn(-c2ccc3c(c2)COc2nc(NC4CCNCC4)ccc2-3)c1] |
| 175 | 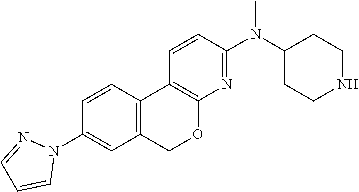 [SMILES: CN(c1ccc2c(n1)OCc1cc(-n3cccn3)ccc1-2)C1CCNCC1] |
| 176 | 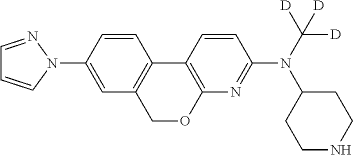 [SMILES: [2H]C([2H])([2H])N(c1ccc2c(n1)OCc1cc(-n3cccn3)ccc1-2)C1CCNCC1] |
| 177 | 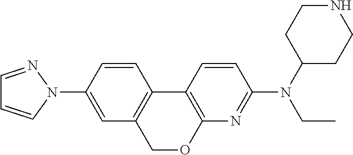 [SMILES: CCN(c1ccc2c(n1)OCc1cc(-n3cccn3)ccc1-2)C1CCNCC1] |
| 178 | 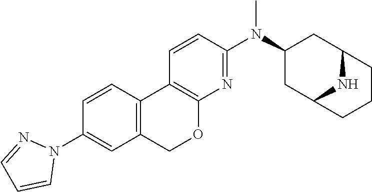 [SMILES: CN(c1ccc2c(n1)OCc1cc(-n3cccn3)ccc1-2)[C@@H]1C[C@H]2CCC[C@@H](C1)N2] |
| 179 | 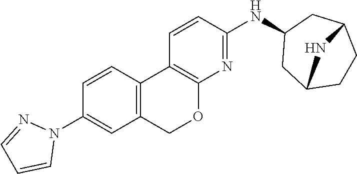 [SMILES: c1cnn(-c2ccc3c(c2)COc2nc(N[C@@H]4C[C@H]5CC[C@@H](C4)N5)ccc2-3)c1] |
| 180 | 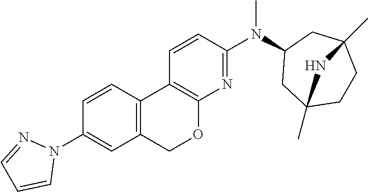 [SMILES: CN(c1ccc2c(n1)OCc1cc(-n3cccn3)ccc1-2)[C@H]1C[C@]2(C)CC[C@](C)(C1)N2] |
| 181 | 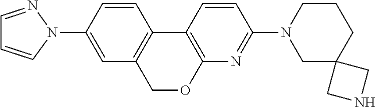 [SMILES: c1cnn(-c2ccc3c(c2)COc2nc(N4CCCC5(CNC5)C4)ccc2-3)c1] |
| 182 | 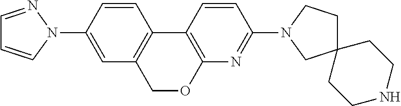 [SMILES: c1cnn(-c2ccc3c(c2)COc2nc(N4CCC5(CCNCC5)C4)ccc2-3)c1] |
| 183 | 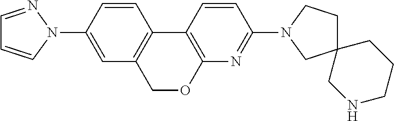 [SMILES: c1cnn(-c2ccc3c(c2)COc2nc(N4CCC5(CCCNC5)C4)ccc2-3)c1] |
| 184 | 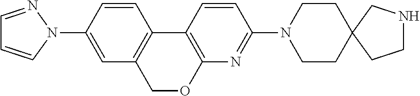 [SMILES: c1cnn(-c2ccc3c(c2)COc2nc(N4CCC5(CCNC5)CC4)ccc2-3)c1] |
| 185 | 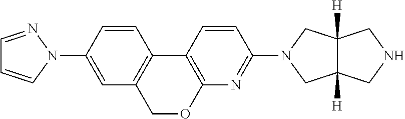 [SMILES: c1cnn(-c2ccc3c(c2)COc2nc(N4C[C@H]5CNC[C@H]5C4)ccc2-3)c1] |
| 186 | 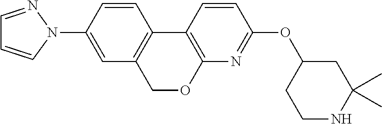 [SMILES: CC1(C)CC(Oc2ccc3c(n2)OCc2cc(-n4cccn4)ccc2-3)CCN1] |
| 187 | 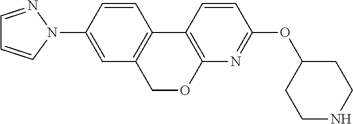 [SMILES: c1cnn(-c2ccc3c(c2)COc2nc(OC4CCNCC4)ccc2-3)c1] |
| 188 | 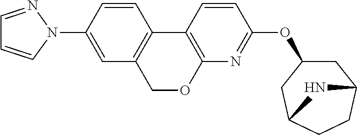 [SMILES: c1cnn(-c2ccc3c(c2)COc2nc(O[C@@H]4C[C@H]5CC[C@@H](C4)N5)ccc2-3)c1] |
| 189 | 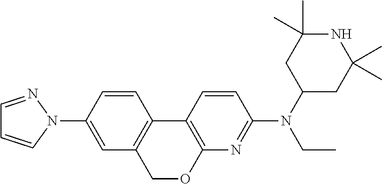 [SMILES: CCN(c1ccc2c(n1)OCc1cc(-n3cccn3)ccc1-2)C1CC(C)(C)NC(C)(C)C1] |
| 190 | 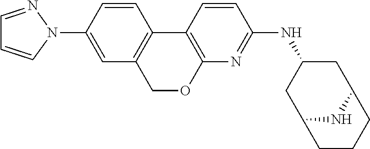 [SMILES: c1cnn(-c2ccc3c(c2)COc2nc(N[C@@H]4C[C@H]5CCC[C@@H](C4)N5)ccc2-3)c1] |
| 191 | 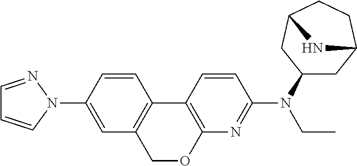 [SMILES: CCN(c1ccc2c(n1)OCc1cc(-n3cccn3)ccc1-2)[C@@H]1C[C@H]2CC[C@@H](C1)N2] |
| 192 | 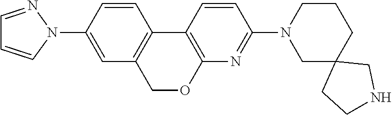 [SMILES: c1cnn(-c2ccc3c(c2)COc2nc(N4CCCC5(CCNC5)C4)ccc2-3)c1] |
| 193 | 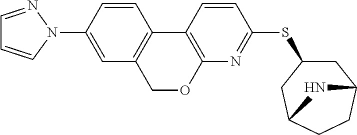 [SMILES: c1cnn(-c2ccc3c(c2)COc2nc(S[C@@H]4C[C@H]5CC[C@@H](C4)N5)ccc2-3)c1] |
| 194 | 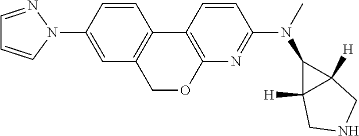 [SMILES: CN(c1ccc2c(n1)OCc1cc(-n3cccn3)ccc1-2)[C@H]1[C@@H]2CNC[C@@H]21] |
| 195 | 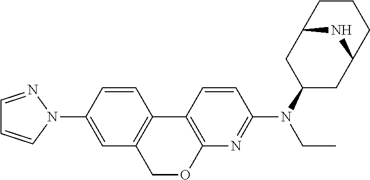 [SMILES: CCN(c1ccc2c(n1)OCc1cc(-n3cccn3)ccc1-2)[C@@H]1C[C@H]2CCC[C@@H](C1)N2] |
| 196 | 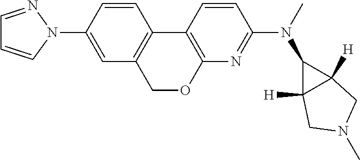 [SMILES: CN1C[C@@H]2[C@H](C1)[C@H]2N(C)c1ccc2c(n1)OCc1cc(-n3cccn3)ccc1-2] |
| 197 | 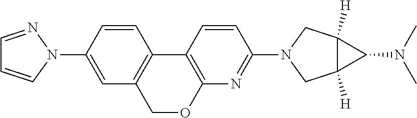 [SMILES: CN(C)[C@H]1[C@@H]2CN(c3ccc4c(n3)OCc3cc(-n5cccn5)ccc3-4)C[C@@H]21] |
| 198 | 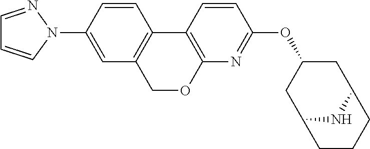 [SMILES: c1cnn(-c2ccc3c(c2)COc2nc(O[C@@H]4C[C@H]5CCC[C@@H](C4)N5)ccc2-3)c1] |
| 199 | 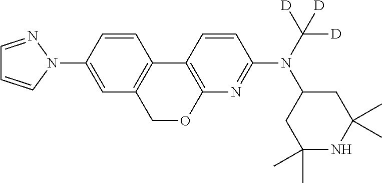 [SMILES: [2H]C([2H])([2H])N(c1ccc2c(n1)OCc1cc(-n3cccn3)ccc1-2)C1CC(C)(C)NC(C)(C)C1] |
| 200 | 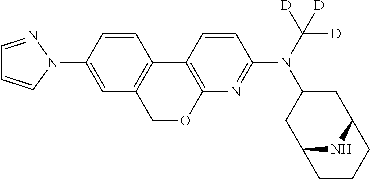 [SMILES: [2H]C([2H])([2H])N(c1ccc2c(n1)OCc1cc(-n3cccn3)ccc1-2)C1C[C@H]2CCC[C@@H](C1)N2] |
| 201 | 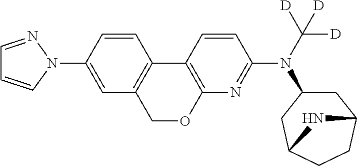 [SMILES: [2H]C([2H])([2H])N(c1ccc2c(n1)OCc1cc(-n3cccn3)ccc1-2)[C@@H]1C[C@H]2CC[C@@H](C1)N2] |
| 202 | 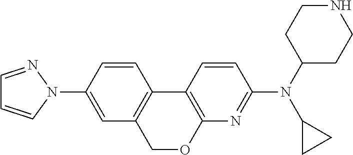 [SMILES: c1cnn(-c2ccc3c(c2)COc2nc(N(C4CCNCC4)C4CC4)ccc2-3)c1] |
| 203 | 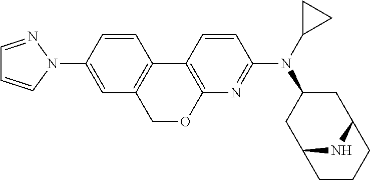 [SMILES: c1cnn(-c2ccc3c(c2)COc2nc(N(C4CC4)[C@@H]4C[C@H]5CCC[C@@H](C4)N5)ccc2-3)c1] |
| 204 | 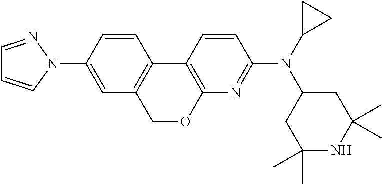 [SMILES: CC1(C)CC(N(c2ccc3c(n2)OCc2cc(-n4cccn4)ccc2-3)C2CC2)CC(C)(C)N1] |
| 205 | 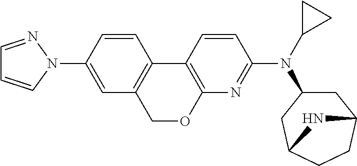 [SMILES: c1cnn(-c2ccc3c(c2)COc2nc(N(C4CC4)[C@@H]4C[C@H]5CC[C@@H](C4)N5)ccc2-3)c1] |
| 206 | 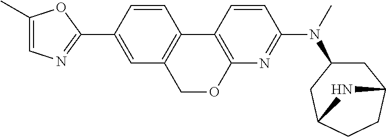 [SMILES: Cc1cnc(-c2ccc3c(c2)COc2nc(N(C)[C@@H]4C[C@H]5CC[C@@H](C4)N5)ccc2-3)o1] |
| 207 | 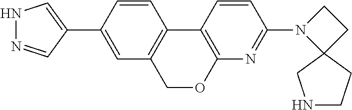 [SMILES: c1cc2c(cc1-c1cn[nH]c1)COc1nc(N3CCC34CCNC4)ccc1-2] |
| 208 | 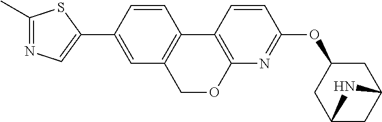 [SMILES: Cc1ncc(-c2ccc3c(c2)COc2nc(O[C@@H]4C[C@H]5C[C@@H](C4)N5)ccc2-3)s1] |
| 209 | 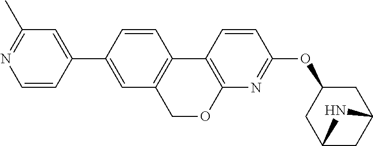 [SMILES: Cc1cc(-c2ccc3c(c2)COc2nc(O[C@@H]4C[C@H]5C[C@@H](C4)N5)ccc2-3)ccn1] |
| 210 | 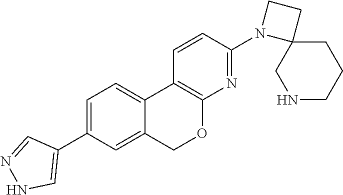 [SMILES: c1cc2c(cc1-c1cn[nH]c1)COc1nc(N3CCC34CCCNC4)ccc1-2] |
| 211 | 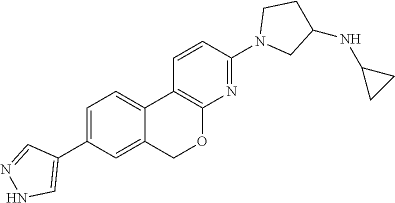 [SMILES: c1cc2c(cc1-c1cn[nH]c1)COc1nc(N3CCC(NC4CC4)C3)ccc1-2] |
| 212 | 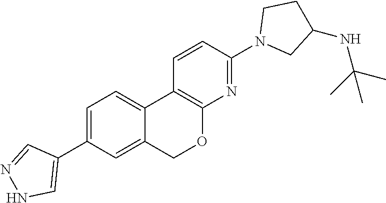 [SMILES: CC(C)(C)NC1CCN(c2ccc3c(n2)OCc2cc(-c4cn[nH]c4)ccc2-3)C1] |
| 213 | 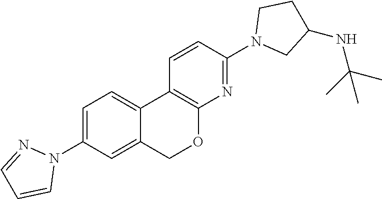 [SMILES: CC(C)(C)NC1CCN(c2ccc3c(n2)OCc2cc(-n4cccn4)ccc2-3)C1] |
| 214 | 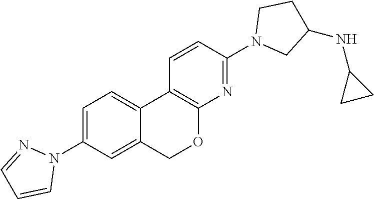 [SMILES: c1cnn(-c2ccc3c(c2)COc2nc(N4CCC(NC5CC5)C4)ccc2-3)c1] |
| 215 | 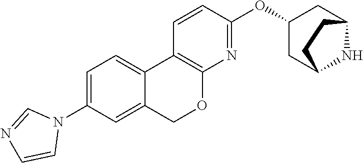 |
| 216 | 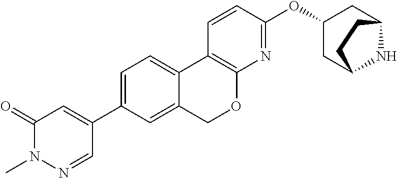 |
| 217 | 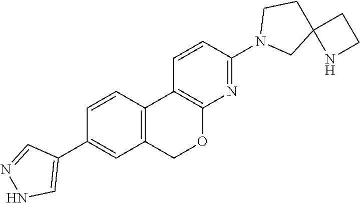 [SMILES: c1cc2c(cc1-c1cn[nH]c1)COc1nc(N3CCC4(CCN4)C3)ccc1-2] |
| 218 | 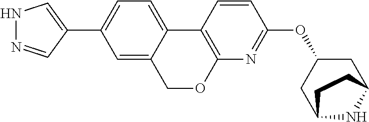 |
| 219 | 601 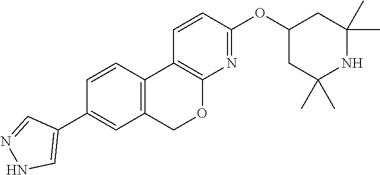 [SMILES: CC1(C)CC(Oc2ccc3c(n2)OCc2cc(-c4cn[nH]c4)ccc2-3)CC(C)(C)N1] |
| 220 | 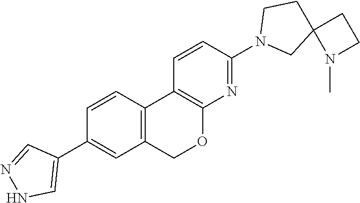 [SMILES: CN1CCC12CCN(c1ccc3c(n1)OCc1cc(-c4cn[nH]c4)ccc1-3)C2] |
| 221 | 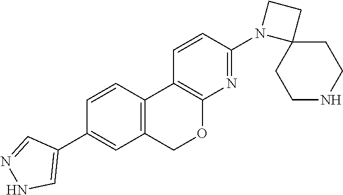 [SMILES: c1cc2c(cc1-c1cn[nH]c1)COc1nc(N3CCC34CCNCC4)ccc1-2] |
| 222 | 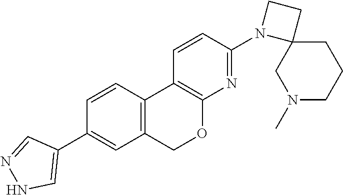 [SMILES: CN1CCCC2(CCN2c2ccc3c(n2)OCc2cc(-c4cn[nH]c4)ccc2-3)C1] |
| 223 | 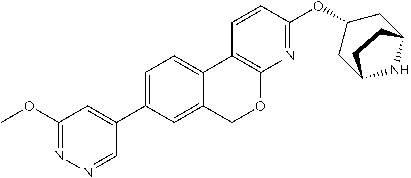 |
| 224 | 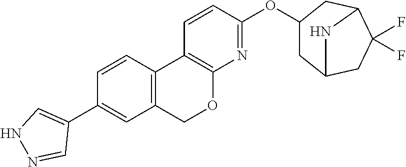 [SMILES: FC1(F)CC2CC(Oc3ccc4c(n3)OCc3cc(-c5cn[nH]c5)ccc3-4)CC1N2] |
| 225 | 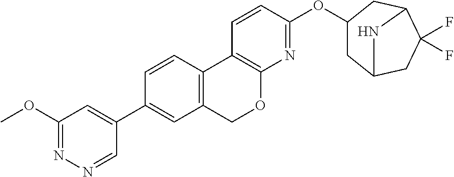 [SMILES: COc1cc(-c2ccc3c(c2)COc2nc(OC4CC5CC(F)(F)C(C4)N5)ccc2-3)cnn1] |
| 226 | 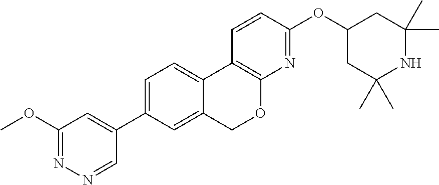 |
| 227 | 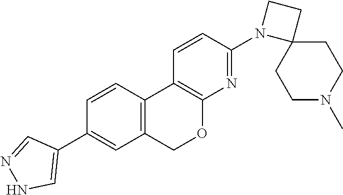 [SMILES: CN1CCC2(CC1)CCN2c1ccc2c(n1)OCc1cc(-c3cn[nH]c3)ccc1-2] |
| 228 | 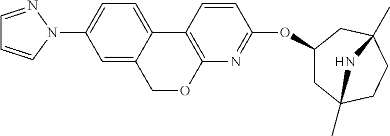 [SMILES: C[C@]12CC[C@](C)(C[C@@H](Oc3ccc4c(n3)OCc3cc(-n5cccn5)ccc3-4)C1)N2] |
| 229 | 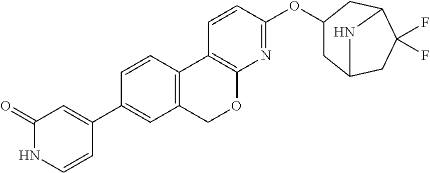 [SMILES: O=c1cc(-c2ccc3c(c2)COc2nc(OC4CC5CC(F)(F)C(C4)N5)ccc2-3)cc[nH]1] |
| 230 | 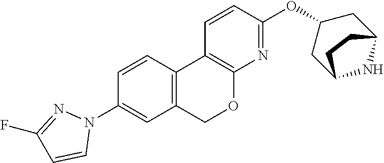 |
| 231 | 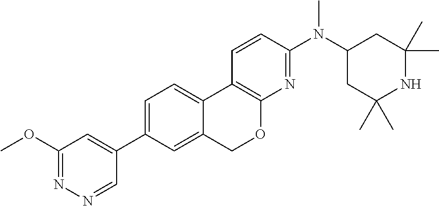 |
| 232 | 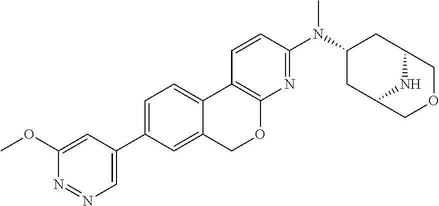 [SMILES: COc1cc(-c2ccc3c(c2)COc2nc(N(C)[C@@H]4C[C@H]5COC[C@@H](C4)N5)ccc2-3)cnn1] |
| 233 | 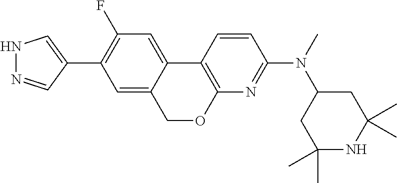 [SMILES: CN(c1ccc2c(n1)OCc1cc(-c3cn[nH]c3)c(F)cc1-2)C1CC(C)(C)NC(C)(C)C1] |
| 234 | 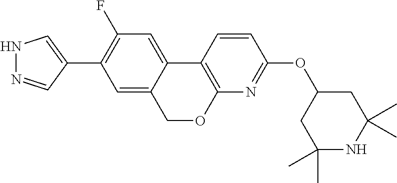 [SMILES: CC1(C)CC(Oc2ccc3c(n2)OCc2cc(-c4cn[nH]c4)c(F)cc2-3)CC(C)(C)N1] |
| 235 | 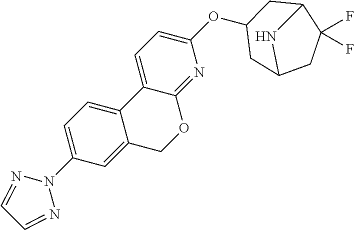 [SMILES: FC1(F)CC2CC(Oc3ccc4c(n3)OCc3cc(-n5nccn5)ccc3-4)CC1N2] |
| 236 | 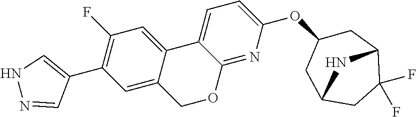 [SMILES: Fc1cc2c(cc1-c1cn[nH]c1)COc1nc(O[C@@H]3C[C@H]4CC(F)(F)[C@@H](C3)N4)ccc1-2] |
| 237 | 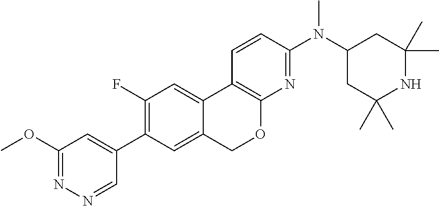 |
| 238 | 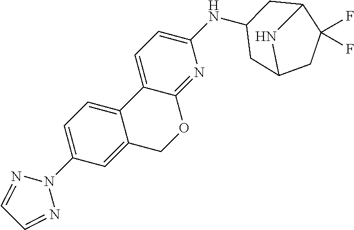 [SMILES: FC1(F)CC2CC(Nc3ccc4c(n3)OCc3cc(-n5nccn5)ccc3-4)CC1N2] |
| 239 | 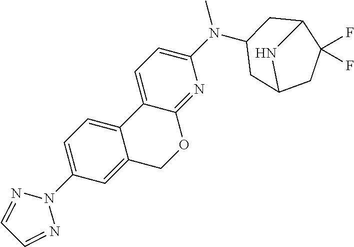 [SMILES: CN(c1ccc2c(n1)OCc1cc(-n3nccn3)ccc1-2)C1CC2CC(F)(F)C(C1)N2] |
| 240 | 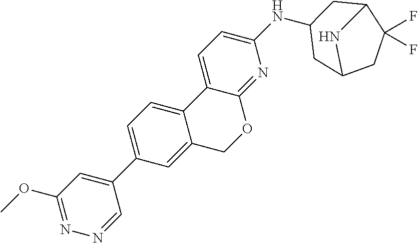 [SMILES: COc1cc(-c2ccc3c(c2)COc2nc(NC4CC5CC(F)(F)C(C4)N5)ccc2-3)cnn1] |
| 241 | 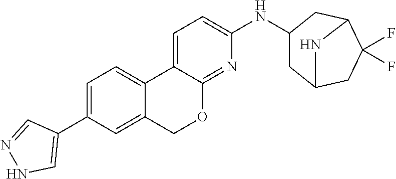 [SMILES: FC1(F)CC2CC(Nc3ccc4c(n3)OCc3cc(-c5cn[nH]c5)ccc3-4)CC1N2] |
| 242 | 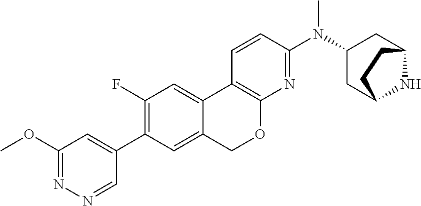 |
| 243 | 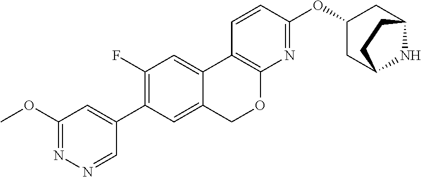 |
| 244 | 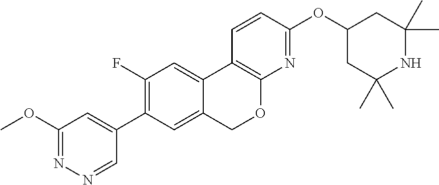 |
| 245 | 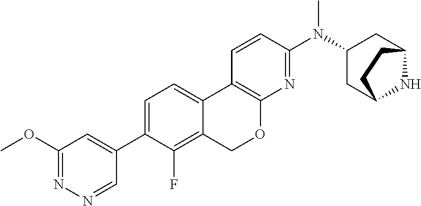 |
| 246 | 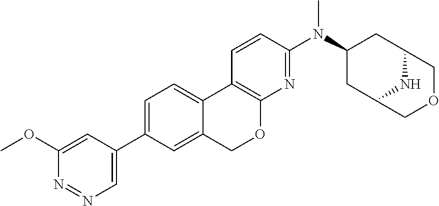 [SMILES: COc1cc(-c2ccc3c(c2)COc2nc(N(C)[C@H]4C[C@H]5COC[C@@H](C4)N5)ccc2-3)cnn1] |
| 247 | 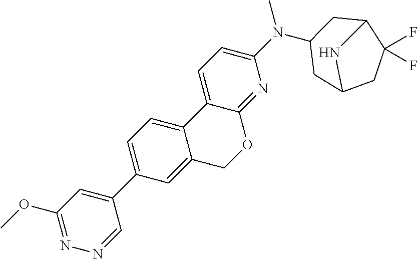 [SMILES: COc1cc(-c2ccc3c(c2)COc2nc(N(C)C4CC5CC(F)(F)C(C4)N5)ccc2-3)cnn1] |
| 248 | 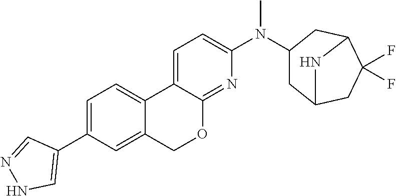 [SMILES: CN(c1ccc2c(n1)OCc1cc(-c3cn[nH]c3)ccc1-2)C1CC2CC(F)(F)C(C1)N2] |
| 249 | 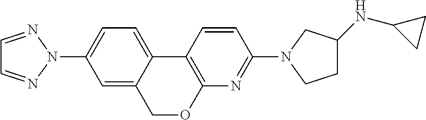 [SMILES: c1cnn(-c2ccc3c(c2)COc2nc(N4CCC(NC5CC5)C4)ccc2-3)n1] |
| 250 | 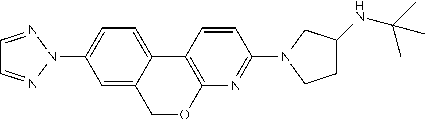 [SMILES: CC(C)(C)NC1CCN(c2ccc3c(n2)OCc2cc(-n4nccn4)ccc2-3)C1] |
| 251 | 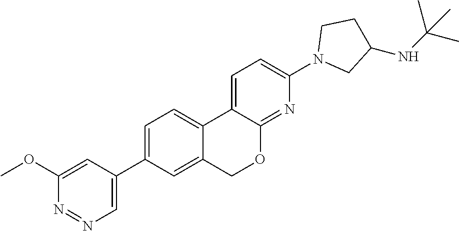 [SMILES: COc1cc(-c2ccc3c(c2)COc2nc(N4CCC(NC(C)(C)C)C4)ccc2-3)cnn1] |
| 252 | 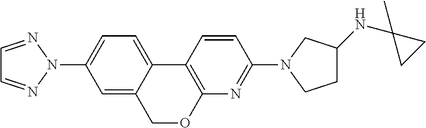 [SMILES: CC1(NC2CCN(c3ccc4c(n3)OCc3cc(-n5nccn5)ccc3-4)C2)CC1] |
| 253 | 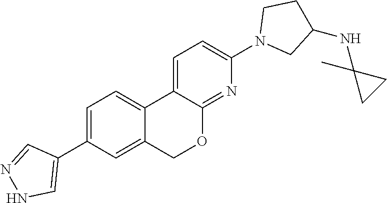 [SMILES: CC1(NC2CCN(c3ccc4c(n3)OCc3cc(-c5cn[nH]c5)ccc3-4)C2)CC1] |
| 254 | 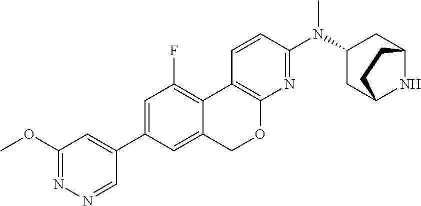 |
| 255 | 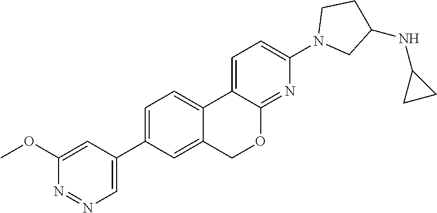 [SMILES: COc1cc(-c2ccc3c(c2)COc2nc(N4CCC(NC5CC5)C4)ccc2-3)cnn1] |
| 256 | 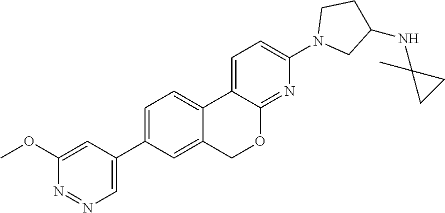 [SMILES: COc1cc(-c2ccc3c(c2)COc2nc(N4CCC(NC5(C)CC5)C4)ccc2-3)cnn1] |
| 257 | 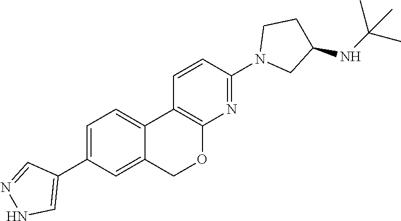 [SMILES: CC(C)(C)N[C@@H]1CCN(c2ccc3c(n2)OCc2cc(-c4cn[nH]c4)ccc2-3)C1] |
| 258 | 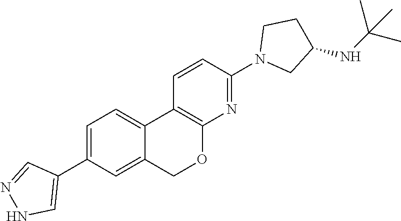 [SMILES: CC(C)(C)N[C@H]1CCN(c2ccc3c(n2)OCc2cc(-c4cn[nH]c4)ccc2-3)C1] |
| 259 | 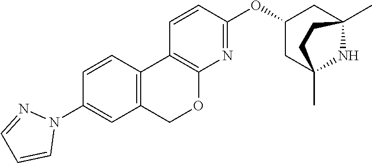 |
| 260 | 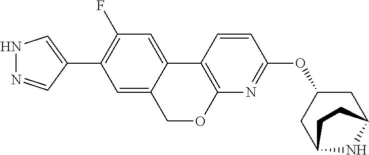 |
| 261 | 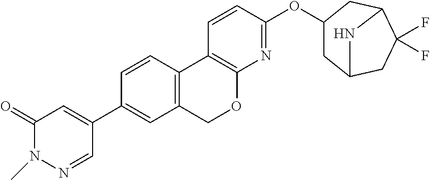 [SMILES: Cn1ncc(-c2ccc3c(c2)COc2nc(OC4CC5CC(F)(F)C(C4)N5)ccc2-3)cc1=O] |
| 262 | 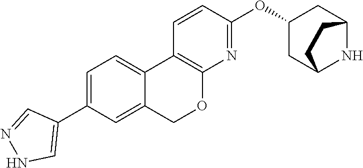 |
| 263 | 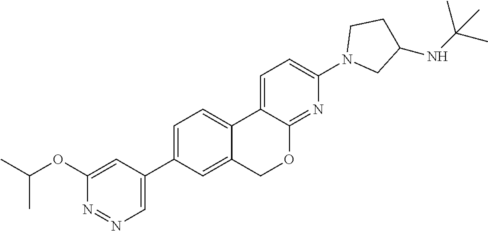 [SMILES: CC(C)Oc1cc(-c2ccc3c(c2)COc2nc(N4CCC(NC(C)(C)C)C4)ccc2-3)cnn1] |
| 264 | 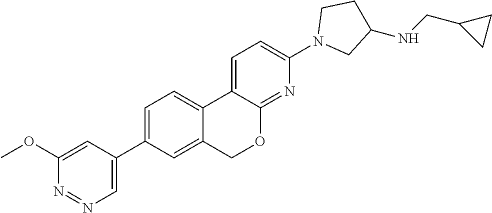 [SMILES: COc1cc(-c2ccc3c(c2)COc2nc(N4CCC(NCC5CC5)C4)ccc2-3)cnn1] |
| 265 | 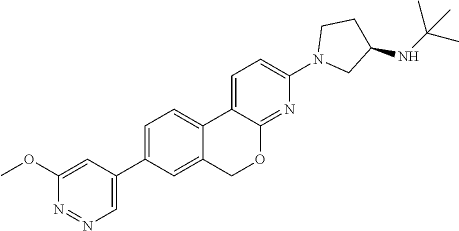 [SMILES: COc1cc(-c2ccc3c(c2)COc2nc(N4CC[C@@H](NC(C)(C)C)C4)ccc2-3)cnn1] |
| 266 | 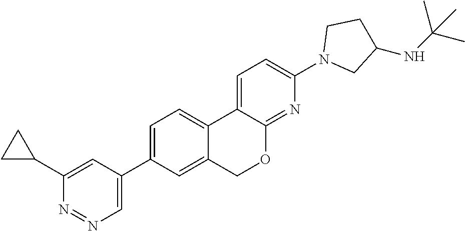 [SMILES: CC(C)(C)NC1CCN(c2ccc3c(n2)OCc2cc(-c4cnnc(C5CC5)c4)ccc2-3)C1] |
| 267 | 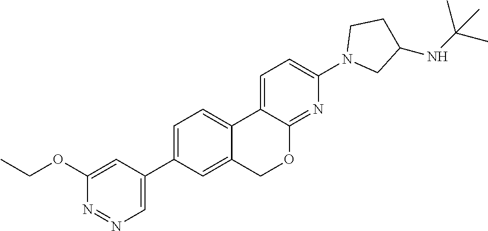 [SMILES: CCOc1cc(-c2ccc3c(c2)COc2nc(N4CCC(NC(C)(C)C)C4)ccc2-3)cnn1] |
| 268 | 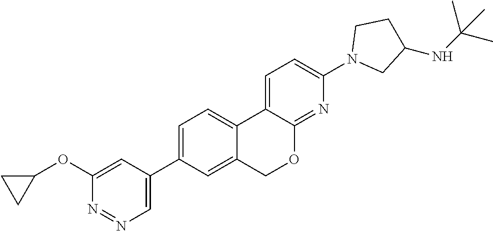 [SMILES: CC(C)(C)NC1CCN(c2ccc3c(n2)OCc2cc(-c4cnnc(OC5CC5)c4)ccc2-3)C1] |
| 269 | 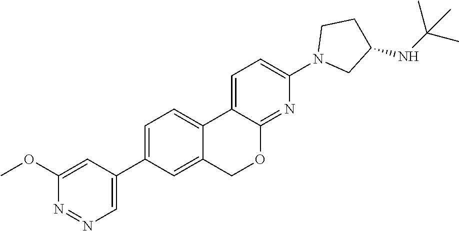 [SMILES: COc1cc(-c2ccc3c(c2)COc2nc(N4CC[C@H](NC(C)(C)C)C4)ccc2-3)cnn1] |
| 270 | 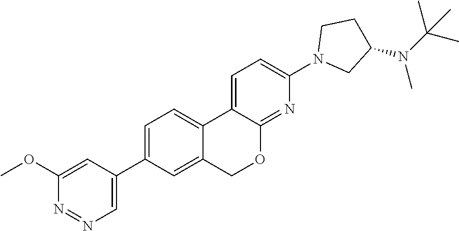 [SMILES: COc1cc(-c2ccc3c(c2)COc2nc(N4CC[C@H](N(C)C(C)(C)C)C4)ccc2-3)cnn1] |
| 271 | 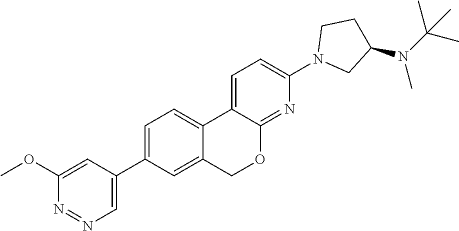 [SMILES: COc1cc(-c2ccc3c(c2)COc2nc(N4CC[C@@H](N(C)C(C)(C)C)C4)ccc2-3)cnn1] |
| 272 | 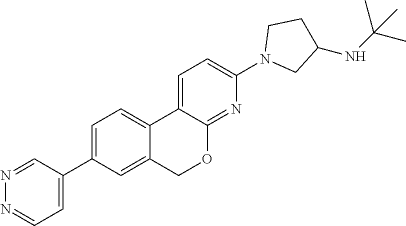 [SMILES: CC(C)(C)NC1CCN(c2ccc3c(n2)OCc2cc(-c4ccnnc4)ccc2-3)C1] |
| 273 | 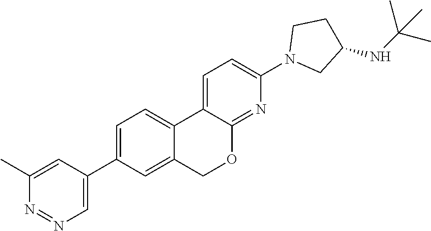 [SMILES: Cc1cc(-c2ccc3c(c2)COc2nc(N4CC[C@H](NC(C)(C)C)C4)ccc2-3)cnn1] |
| 274 | 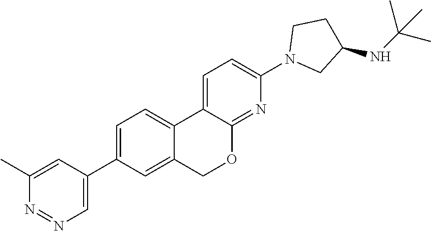 [SMILES: Cc1cc(-c2ccc3c(c2)COc2nc(N4CC[C@@H](NC(C)(C)C)C4)ccc2-3)cnn1] |
| 275 | 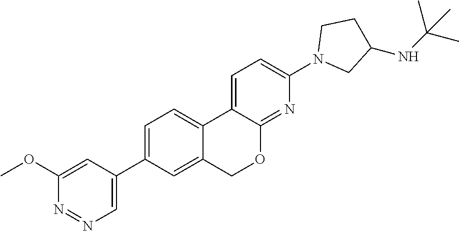 [SMILES: COc1cc(-c2ccc3c(c2)COc2nc(N4CCC(NC(C)(C)C)C4)ccc2-3)cnn1] |
| 276 | 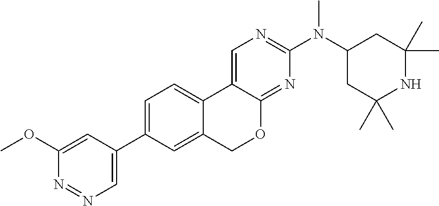 |
| 277 | 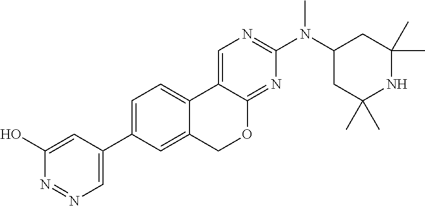 [SMILES: CN(c1ncc2c(n1)OCc1cc(-c3cnnc(O)c3)ccc1-2)C1CC(C)(C)NC(C)(C)C1] |
| 278 | 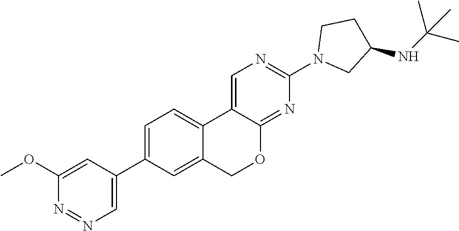 [SMILES: COc1cc(-c2ccc3c(c2)COc2nc(N4CC[C@@H](NC(C)(C)C)C4)ncc2-3)cnn1] |
| 279 | 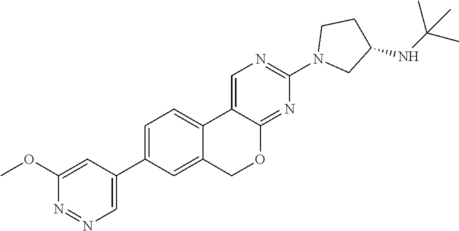 [SMILES: COc1cc(-c2ccc3c(c2)COc2nc(N4CC[C@H](NC(C)(C)C)C4)ncc2-3)cnn1] |
| 280 | 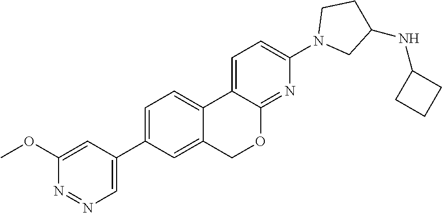 [SMILES: COc1cc(-c2ccc3c(c2)COc2nc(N4CCC(NC5CCC5)C4)ccc2-3)cnn1] |
| 281 | 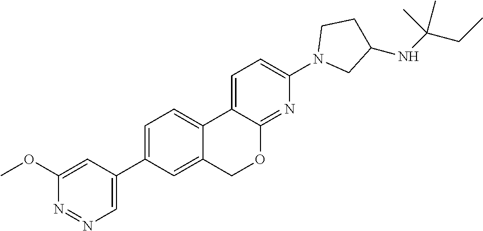 [SMILES: CCC(C)(C)NC1CCN(c2ccc3c(n2)OCc2cc(-c4cnnc(OC)c4)ccc2-3)C1] |
| 282 | 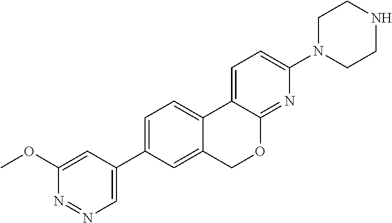 [SMILES: COc1cc(-c2ccc3c(c2)COc2nc(N4CCNCC4)ccc2-3)cnn1] |
| 283 | 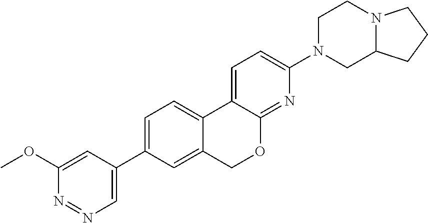 [SMILES: COc1cc(-c2ccc3c(c2)COc2nc(N4CCN5CCCC5C4)ccc2-3)cnn1] |
| 284 | 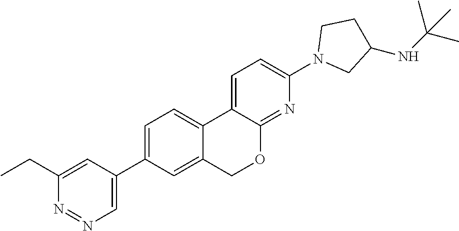 [SMILES: CCc1cc(-c2ccc3c(c2)COc2nc(N4CCC(NC(C)(C)C)C4)ccc2-3)cnn1] |
| 285 | 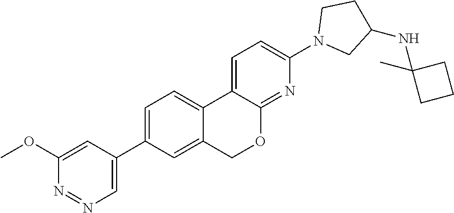 [SMILES: COc1cc(-c2ccc3c(c2)COc2nc(N4CCC(NC5(C)CCC5)C4)ccc2-3)cnn1] |
| 286 | 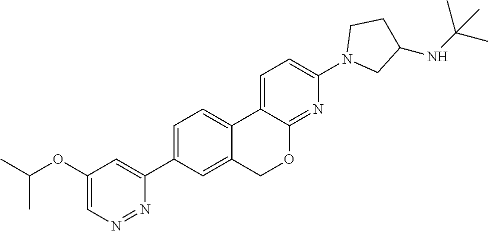 [SMILES: CC(C)Oc1cnnc(-c2ccc3c(c2)COc2nc(N4CCC(NC(C)(C)C)C4)ccc2-3)c1] |
| 287 | 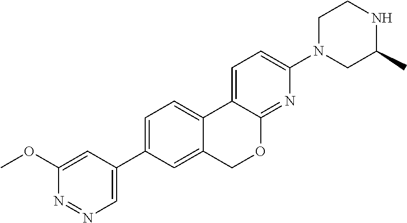 |
| 288 | 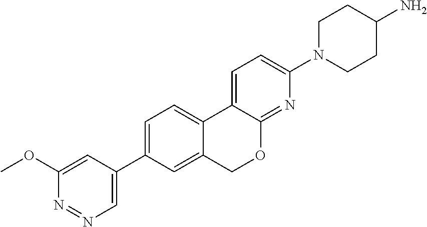 [SMILES: COc1cc(-c2ccc3c(c2)COc2nc(N4CCC(N)CC4)ccc2-3)cnn1] |
| 289 | 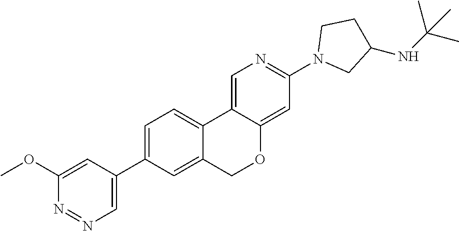 [SMILES: COc1cc(-c2ccc3c(c2)COc2cc(N4CCC(NC(C)(C)C)C4)ncc2-3)cnn1] |
| 290 | 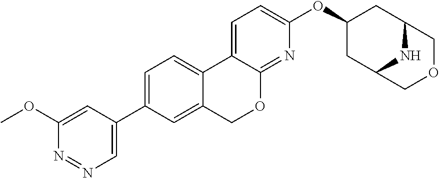 [SMILES: COc1cc(-c2ccc3c(c2)COc2nc(O[C@@H]4C[C@H]5COC[C@@H](C4)N5)ccc2-3)cnn1] |
| 291 | 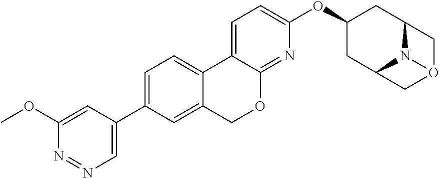 |
| 292 | 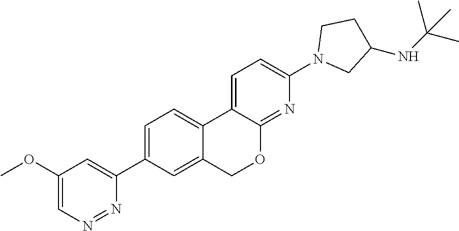 [SMILES: COc1cnnc(-c2ccc3c(c2)COc2nc(N4CCC(NC(C)(C)C)C4)ccc2-3)c1] |
| 293 | 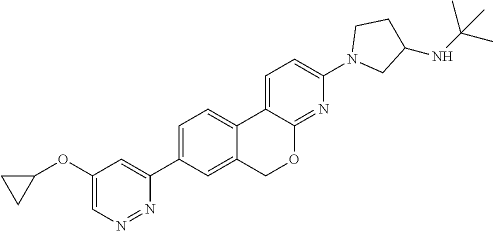 [SMILES: CC(C)(C)NC1CCN(c2ccc3c(n2)OCc2cc(-c4cc(OC5CC5)cnn4)ccc2-3)C1] |
| 294 | 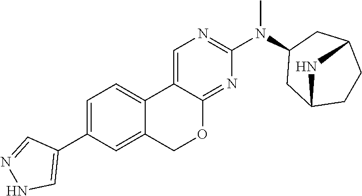 [SMILES: CN(c1ncc2c(n1)OCc1cc(-c3cn[nH]c3)ccc1-2)[C@@H]1C[C@H]2CC[C@@H](C1)N2] |
| 295 | 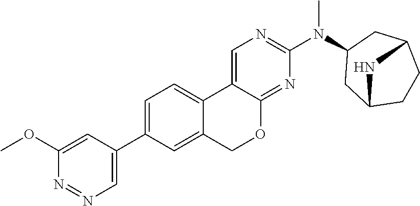 [SMILES: COc1cc(-c2ccc3c(c2)COc2nc(N(C)[C@@H]4C[C@H]5CC[C@@H](C4)N5)ncc2-3)cnn1] |
| 296 | 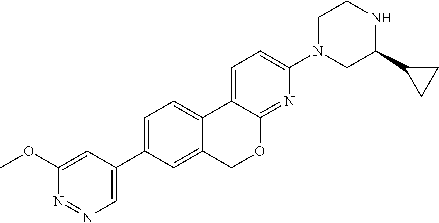 [SMILES: COc1cc(-c2ccc3c(c2)COc2nc(N4CCN[C@@H](C5CC5)C4)ccc2-3)cnn1] |
| 297 | 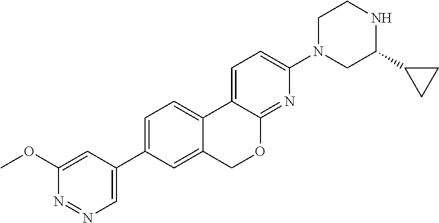 [SMILES: COc1cc(-c2ccc3c(c2)COc2nc(N4CCN[C@H](C5CC5)C4)ccc2-3)cnn1] |
| 298 | 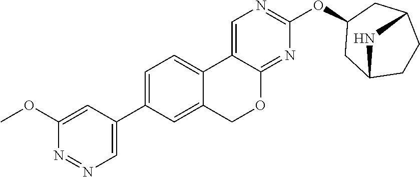 [SMILES: COc1cc(-c2ccc3c(c2)COc2nc(O[C@@H]4C[C@H]5CC[C@@H](C4)N5)ncc2-3)cnn1] |
| 299 | 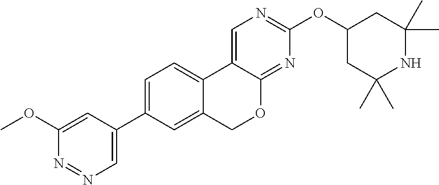 |
| 300 | 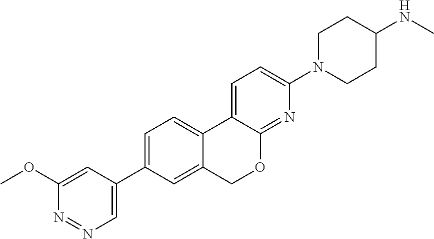 |
| 301 | 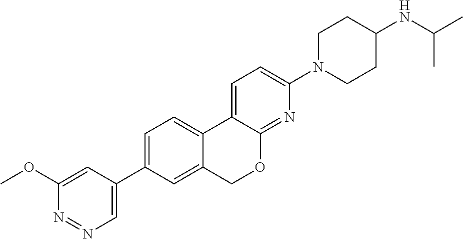 [SMILES: COc1cc(-c2ccc3c(c2)COc2nc(N4CCC(NC(C)C)CC4)ccc2-3)cnn1] |
| 302 | 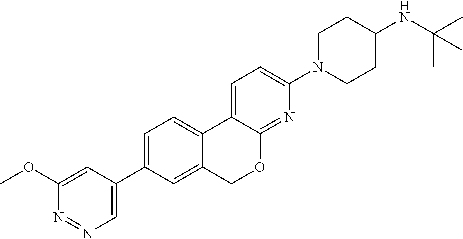 [SMILES: COc1cc(-c2ccc3c(c2)COc2nc(N4CCC(NC(C)(C)C)CC4)ccc2-3)cnn1] |
| 303 | 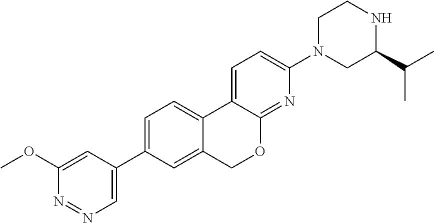 [SMILES: COc1cc(-c2ccc3c(c2)COc2nc(N4CCN[C@@H](C(C)C)C4)ccc2-3)cnn1] |
| 304 | 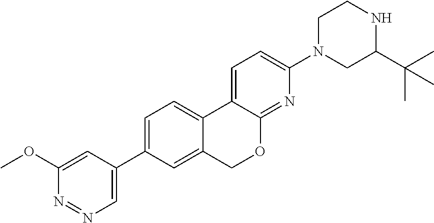 [SMILES: COc1cc(-c2ccc3c(c2)COc2nc(N4CCNC(C(C)(C)C)C4)ccc2-3)cnn1] |
| 305 | 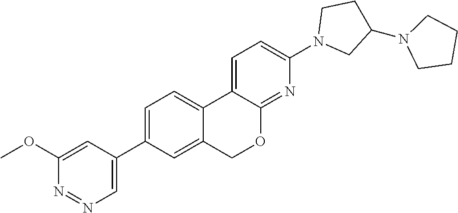 [SMILES: COc1cc(-c2ccc3c(c2)COc2nc(N4CCC(N5CCCC5)C4)ccc2-3)cnn1] |
| 306 | 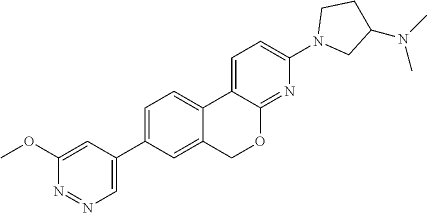 |
| 307 | 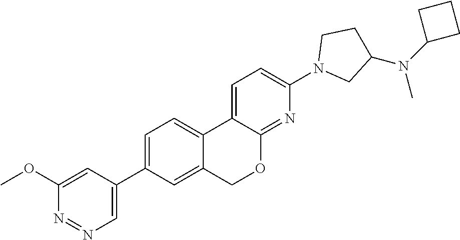 [SMILES: COc1cc(-c2ccc3c(c2)COc2nc(N4CCC(N(C)C5CCC5)C4)ccc2-3)cnn1] |
| 308 | 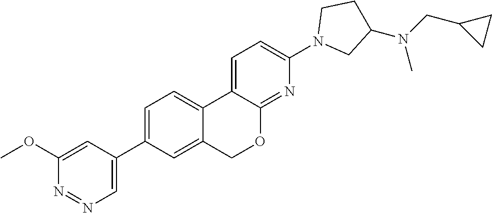 [SMILES: COc1cc(-c2ccc3c(c2)COc2nc(N4CCC(N(C)CC5CC5)C4)ccc2-3)cnn1] |
| 309 | 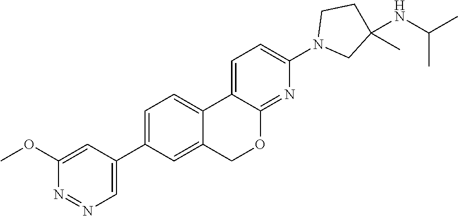 [SMILES: COc1cc(-c2ccc3c(c2)COc2nc(N4CCC(C)(NC(C)C)C4)ccc2-3)cnn1] |
| 310 | 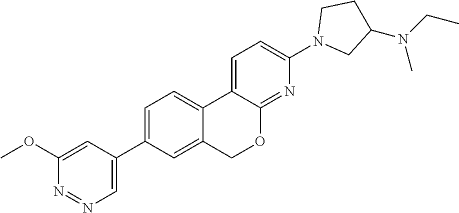 [SMILES: CCN(C)C1CCN(c2ccc3c(n2)OCc2cc(-c4cnnc(OC)c4)ccc2-3)C1] |
| 311 | 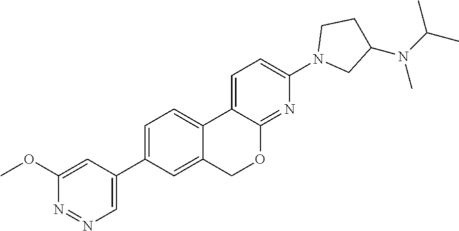 [SMILES: COc1cc(-c2ccc3c(c2)COc2nc(N4CCC(N(C)C(C)C)C4)ccc2-3)cnn1] |
| 312 | 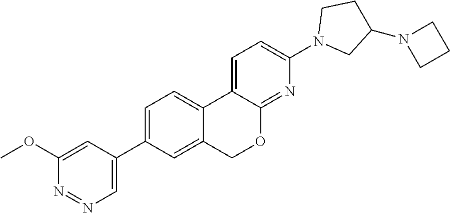 [SMILES: COc1cc(-c2ccc3c(c2)COc2nc(N4CCC(N5CCC5)C4)ccc2-3)cnn1] |
| 313 | 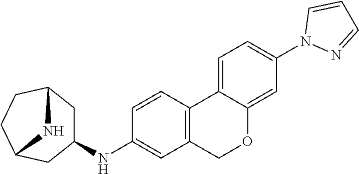 [SMILES: c1cnn(-c2ccc3c(c2)OCc2cc(N[C@@H]4C[C@H]5CC[C@@H](C4)N5)ccc2-3)c1] |
| 314 | 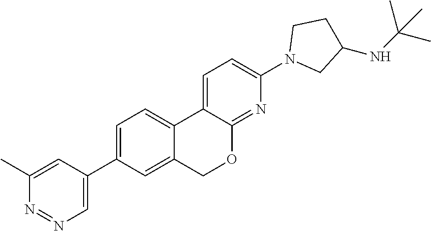 [SMILES: Cc1cc(-c2ccc3c(c2)COc2nc(N4CCC(NC(C)(C)C)C4)ccc2-3)cnn1] |
| 315 | 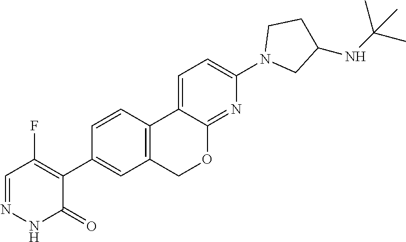 [SMILES: CC(C)(C)NC1CCN(c2ccc3c(n2)OCc2cc(-c4c(F)cn[nH]c4=O)ccc2-3)C1] |
| 316 | 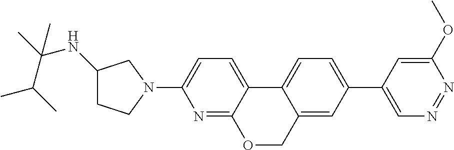 [SMILES: COc1cc(-c2ccc3c(c2)COc2nc(N4CCC(NC(C)(C)C(C)C)C4)ccc2-3)cnn1] |
| 317 | 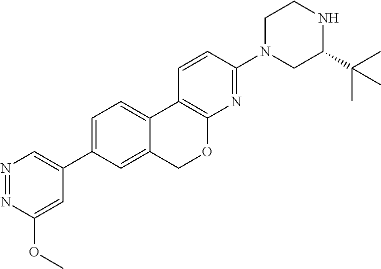 [SMILES: COc1cc(-c2ccc3c(c2)COc2nc(N4CCN[C@H](C(C)(C)C)C4)ccc2-3)cnn1] |
| 318 | 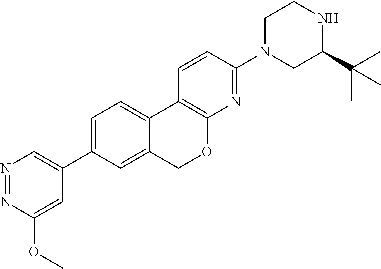 [SMILES: COc1cc(-c2ccc3c(c2)COc2nc(N4CCN[C@@H](C(C)(C)C)C4)ccc2-3)cnn1] |
| 319 | 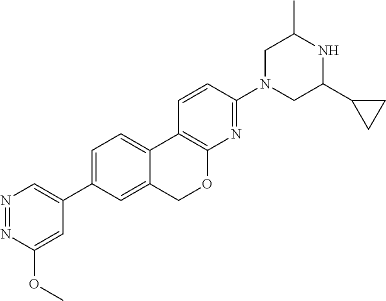 [SMILES: COc1cc(-c2ccc3c(c2)COc2nc(N4CC(C)NC(C5CC5)C4)ccc2-3)cnn1] |
| 320 | 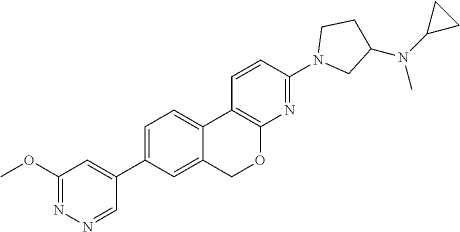 [SMILES: COc1cc(-c2ccc3c(c2)COc2nc(N4CCC(N(C)C5CC5)C4)ccc2-3)cnn1] |
| 321 | 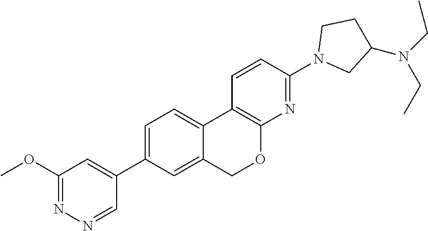 [SMILES: CCN(CC)C1CCN(c2ccc3c(n2)OCc2cc(-c4cnnc(OC)c4)ccc2-3)C1] |
| 322 | 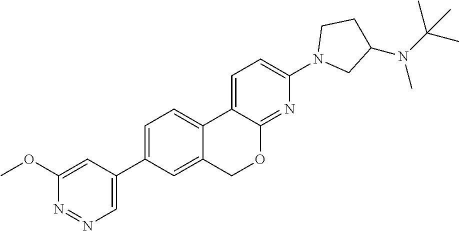 [SMILES: COc1cc(-c2ccc3c(c2)COc2nc(N4CCC(N(C)C(C)(C)C)C4)ccc2-3)cnn1] |
| 323 | 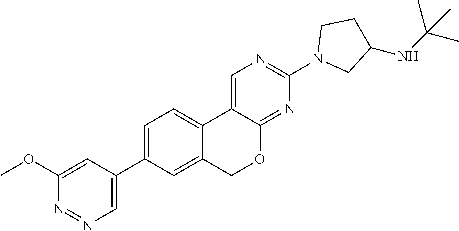 [SMILES: COc1cc(-c2ccc3c(c2)COc2nc(N4CCC(NC(C)(C)C)C4)ncc2-3)cnn1] |
| 324 | 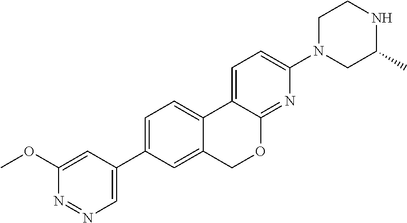 |
| 325 | 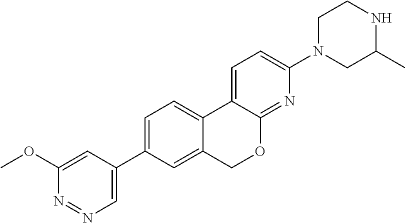 |
| 326 | 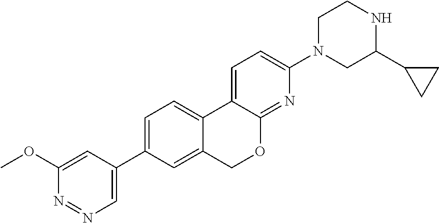 [SMILES: COc1cc(-c2ccc3c(c2)COc2nc(N4CCNC(C5CC5)C4)ccc2-3)cnn1] |
| 327 | 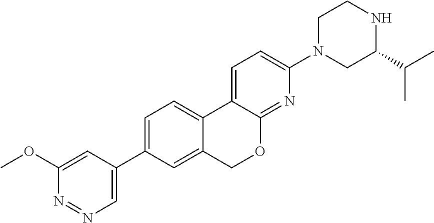 [SMILES: COc1cc(-c2ccc3c(c2)COc2nc(N4CCN[C@H](C(C)C)C4)ccc2-3)cnn1] |
| 328 | 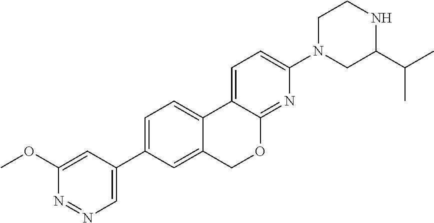 [SMILES: COc1cc(-c2ccc3c(c2)COc2nc(N4CCNC(C(C)C)C4)ccc2-3)cnn1] |
| 329 | 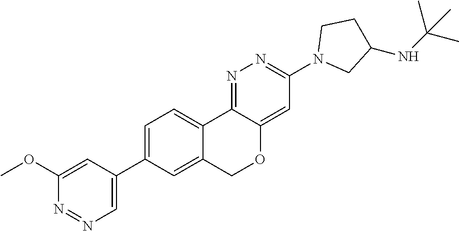 [SMILES: COc1cc(-c2ccc3c(c2)COc2cc(N4CCC(NC(C)(C)C)C4)nnc2-3)cnn1] |
| 330 | 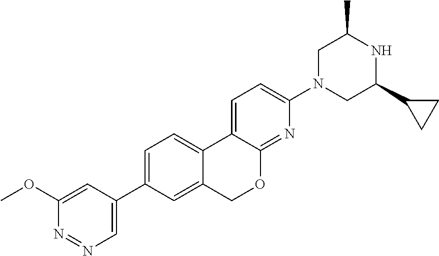 [SMILES: COc1cc(-c2ccc3c(c2)COc2nc(N4C[C@@H](C)N[C@@H](C5CC5)C4)ccc2-3)cnn1] |
| 331 | 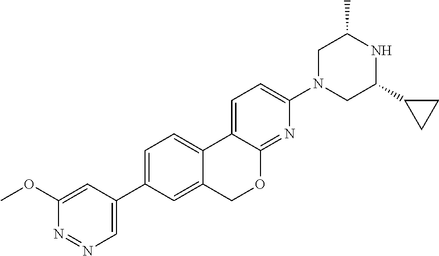 [SMILES: COc1cc(-c2ccc3c(c2)COc2nc(N4C[C@@H](C5CC5)N[C@@H](C)C4)ccc2-3)cnn1] |
| 332 | 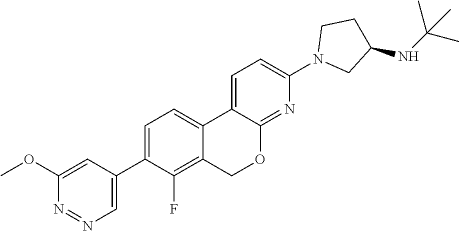 [SMILES: COc1cc(-c2ccc3c(c2F)COc2nc(N4CC[C@@H](NC(C)(C)C)C4)ccc2-3)cnn1] |
| 333 | 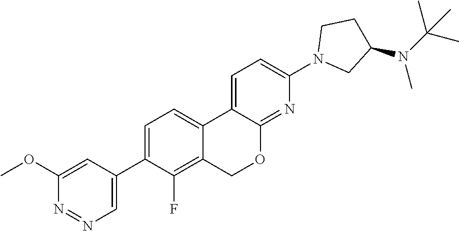 [SMILES: COc1cc(-c2ccc3c(c2F)COc2nc(N4CC[C@@H](N(C)C(C)(C)C)C4)ccc2-3)cnn1] |
| 334 | 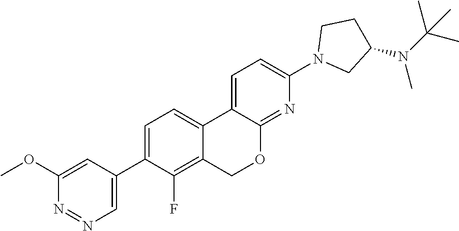 [SMILES: COc1cc(-c2ccc3c(c2F)COc2nc(N4CC[C@H](N(C)C(C)(C)C)C4)ccc2-3)cnn1] |
| 335 | 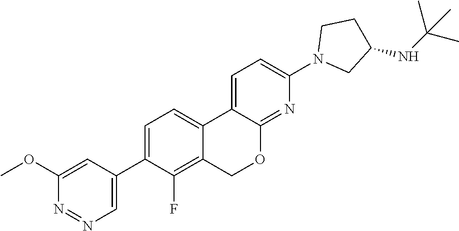 [SMILES: COc1cc(-c2ccc3c(c2F)COc2nc(N4CC[C@H](NC(C)(C)C)C4)ccc2-3)cnn1] |
| 336 | 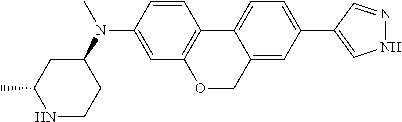 [SMILES: C[C@@H]1C[C@@H](N(C)c2ccc3c(c2)OCc2cc(-c4cn[nH]c4)ccc2-3)CCN1] |
| 337 | 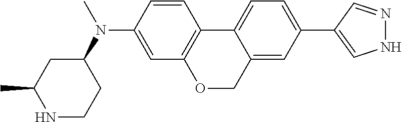 [SMILES: C[C@H]1C[C@@H](N(C)c2ccc3c(c2)OCc2cc(-c4cn[nH]c4)ccc2-3)CCN1] |
| 338 | 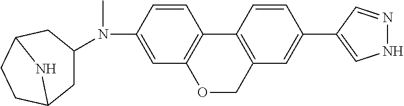 [SMILES: CN(c1ccc2c(c1)OCc1cc(-c3cn[nH]c3)ccc1-2)C1CC2CCC(C1)N2] |
| 339 | 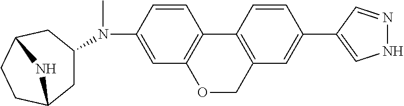 [SMILES: CN(c1ccc2c(c1)OCc1cc(-c3cn[nH]c3)ccc1-2)[C@H]1C[C@H]2CC[C@@H](C1)N2] |
| 340 | 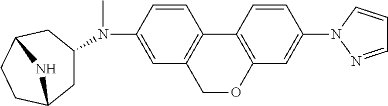 [SMILES: CN(c1ccc2c(c1)COc1cc(-n3cccn3)ccc1-2)[C@H]1C[C@H]2CC[C@@H](C1)N2] |
| 341 | 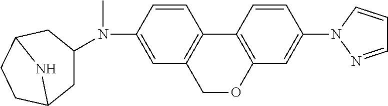 [SMILES: CN(c1ccc2c(c1)COc1cc(-n3cccn3)ccc1-2)C1CC2CCC(C1)N2] |
| 342 | 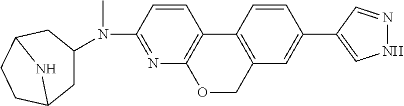 [SMILES: CN(c1ccc2c(n1)OCc1cc(-c3cn[nH]c3)ccc1-2)C1CC2CCC(C1)N2] |
| 343 | 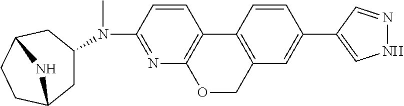 [SMILES: CN(c1ccc2c(n1)OCc1cc(-c3cn[nH]c3)ccc1-2)[C@H]1C[C@H]2CC[C@@H](C1)N2] |
| 344 | 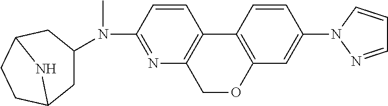 [SMILES: CN(c1ccc2c(n1)COc1cc(-n3cccn3)ccc1-2)C1CC2CCC(C1)N2] |
| 345 | 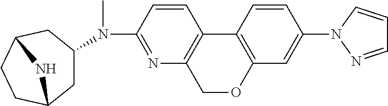 [SMILES: CN(c1ccc2c(n1)COc1cc(-n3cccn3)ccc1-2)[C@H]1C[C@H]2CC[C@@H](C1)N2] |
| 346 | 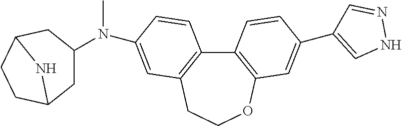 [SMILES: CN(c1ccc2c(c1)CCOc1cc(-c3cn[nH]c3)ccc1-2)C1CC2CCC(C1)N2] |
| 347 | 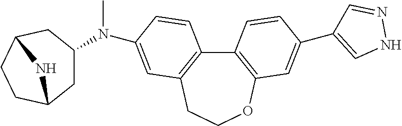 [SMILES: CN(c1ccc2c(c1)CCOc1cc(-c3cn[nH]c3)ccc1-2)[C@H]1C[C@H]2CC[C@@H](C1)N2] |
| 348 | 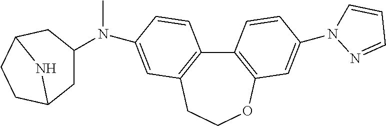 [SMILES: CN(c1ccc2c(c1)CCOc1cc(-n3cccn3)ccc1-2)C1CC2CCC(C1)N2] |
| 349 | 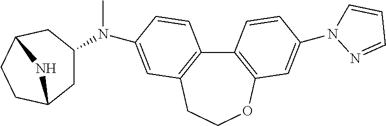 [SMILES: CN(c1ccc2c(c1)CCOc1cc(-n3cccn3)ccc1-2)[C@H]1C[C@H]2CC[C@@H](C1)N2] |
| 350 | 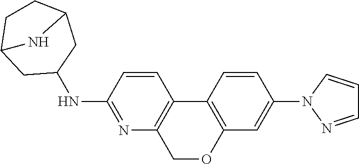 [SMILES: c1cnn(-c2ccc3c(c2)OCc2nc(NC4CC5CCC(C4)N5)ccc2-3)c1] |
| 351 | 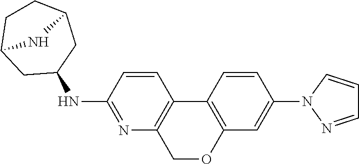 [SMILES: c1cnn(-c2ccc3c(c2)OCc2nc(N[C@H]4C[C@H]5CC[C@@H](C4)N5)ccc2-3)c1] |
| 352 | 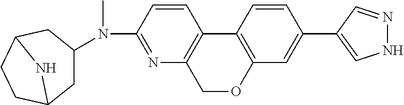 [SMILES: CN(c1ccc2c(n1)COc1cc(-c3cn[nH]c3)ccc1-2)C1CC2CCC(C1)N2] |
| 353 | 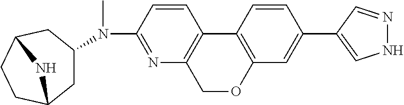 [SMILES: CN(c1ccc2c(n1)COc1cc(-c3cn[nH]c3)ccc1-2)[C@H]1C[C@H]2CC[C@@H](C1)N2] |
| 354 | 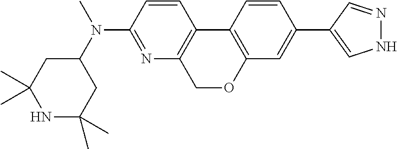 [SMILES: CN(c1ccc2c(n1)COc1cc(-c3cn[nH]c3)ccc1-2)C1CC(C)(C)NC(C)(C)C1] |
| 355 | 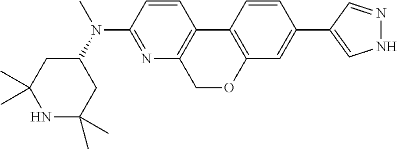 |
| 356 | 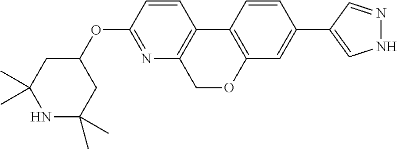 [SMILES: CC1(C)CC(Oc2ccc3c(n2)COc2cc(-c4cn[nH]c4)ccc2-3)CC(C)(C)N1] |
| 357 | 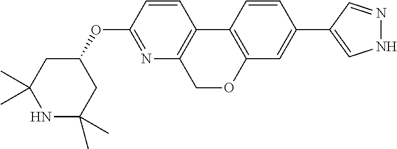 |
| 358 | 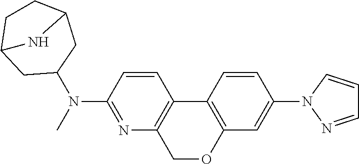 [SMILES: CN(c1ccc2c(n1)COc1cc(-n3cccn3)ccc1-2)C1CC2CCC(C1)N2] |
| 359 | 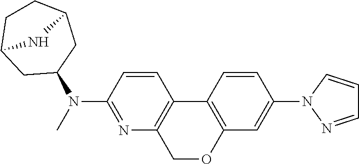 [SMILES: CN(c1ccc2c(n1)COc1cc(-n3cccn3)ccc1-2)[C@H]1C[C@H]2CC[C@@H](C1)N2] |
| 360 | 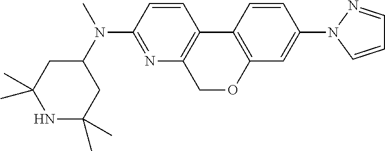 [SMILES: CN(c1ccc2c(n1)COc1cc(-n3cccn3)ccc1-2)C1CC(C)(C)NC(C)(C)C1] |
| 361 | 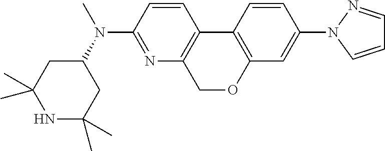 |
| 362 | 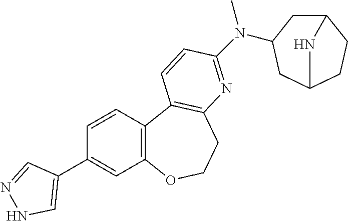 [SMILES: CN(c1ccc2c(n1)CCOc1cc(-c3cn[nH]c3)ccc1-2)C1CC2CCC(C1)N2] |
| 363 | 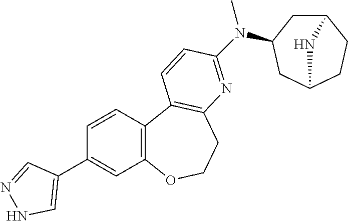 [SMILES: CN(c1ccc2c(n1)CCOc1cc(-c3cn[nH]c3)ccc1-2)[C@H]1C[C@H]2CC[C@@H](C1)N2] |
| 364 | 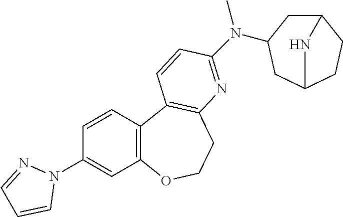 [SMILES: CN(c1ccc2c(n1)CCOc1cc(-n3cccn3)ccc1-2)C1CC2CCC(C1)N2] |
| 365 | 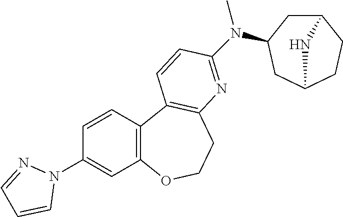 [SMILES: CN(c1ccc2c(n1)CCOc1cc(-n3cccn3)ccc1-2)[C@H]1C[C@H]2CC[C@@H](C1)N2] |
| 366 | 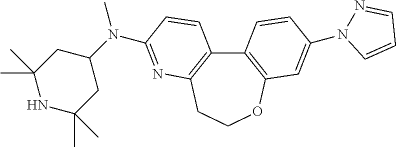 [SMILES: CN(c1ccc2c(n1)CCOc1cc(-n3cccn3)ccc1-2)C1CC(C)(C)NC(C)(C)C1] |
| 367 | 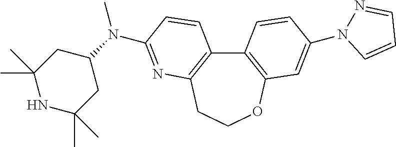 |
| 368 | 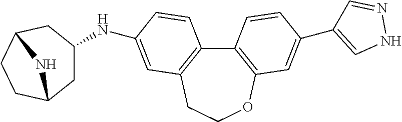 [SMILES: c1cc2c(cc1N[C@H]1C[C@H]3CC[C@@H](C1)N3)CCOc1cc(-c3cn[nH]c3)ccc1-2] |
| 369 | 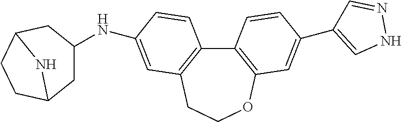 [SMILES: c1cc2c(cc1NC1CC3CCC(C1)N3)CCOc1cc(-c3cn[nH]c3)ccc1-2] |
| 370 | 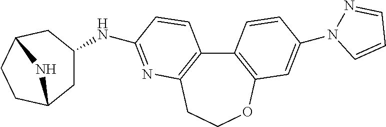 [SMILES: c1cnn(-c2ccc3c(c2)OCCc2nc(N[C@H]4C[C@H]5CC[C@@H](C4)N5)ccc2-3)c1] |
| 371 | 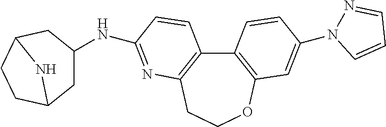 [SMILES: c1cnn(-c2ccc3c(c2)OCCc2nc(NC4CC5CCC(C4)N5)ccc2-3)c1] |
| 372 | 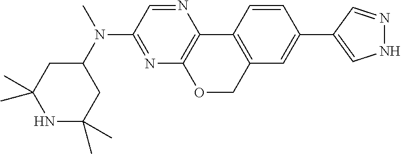 [SMILES: CN(c1cnc2c(n1)OCc1cc(-c3cn[nH]c3)ccc1-2)C1CC(C)(C)NC(C)(C)C1] |
| 373 | 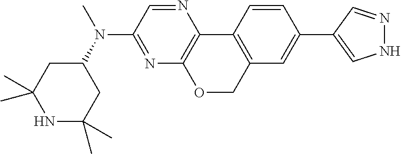 |
| 374 | 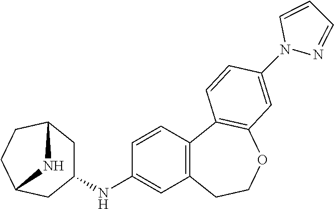 [SMILES: c1cnn(-c2ccc3c(c2)OCCc2cc(N[C@H]4C[C@H]5CC[C@@H](C4)N5)ccc2-3)c1] |
| 375 | 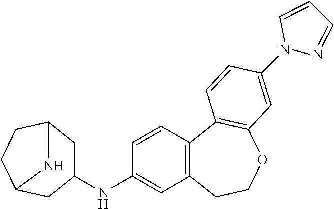 [SMILES: c1cnn(-c2ccc3c(c2)OCCc2cc(NC4CC5CCC(C4)N5)ccc2-3)c1] |
| 376 | 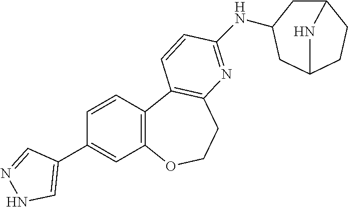 [SMILES: c1cc2c(cc1-c1cn[nH]c1)OCCc1nc(NC3CC4CCC(C3)N4)ccc1-2] |
| 377 | 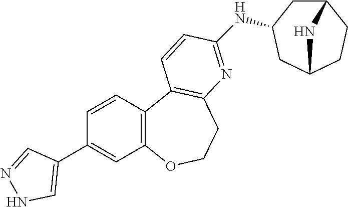 [SMILES: c1cc2c(cc1-c1cn[nH]c1)OCCc1nc(N[C@H]3C[C@H]4CC[C@@H](C3)N4)ccc1-2] |
| 378 | 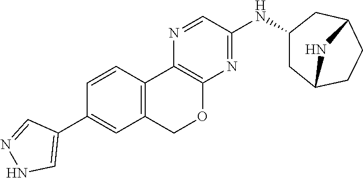 [SMILES: c1cc2c(cc1-c1cn[nH]c1)COc1nc(N[C@H]3C[C@H]4CC[C@@H](C3)N4)cnc1-2] |
| 379 | 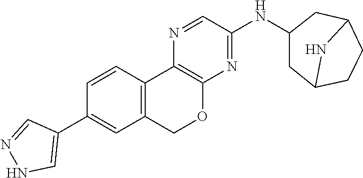 [SMILES: c1cc2c(cc1-c1cn[nH]c1)COc1nc(NC3CC4CCC(C3)N4)cnc1-2] |
| 380 | 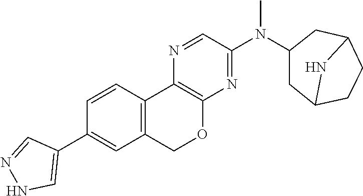 [SMILES: CN(c1cnc2c(n1)OCc1cc(-c3cn[nH]c3)ccc1-2)C1CC2CCC(C1)N2] |
| 381 | 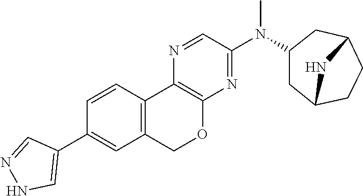 |
| 382 | 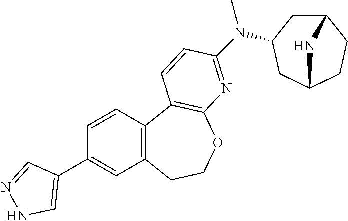 [SMILES: CN(c1ccc2c(n1)OCCc1cc(-c3cn[nH]c3)ccc1-2)[C@H]1C[C@H]2CC[C@@H](C1)N2] |
| 383 | 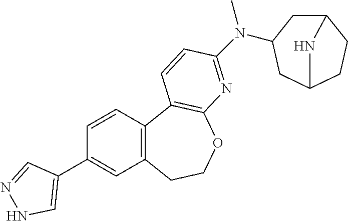 [SMILES: CN(c1ccc2c(n1)OCCc1cc(-c3cn[nH]c3)ccc1-2)C1CC2CCC(C1)N2] |
| 384 | 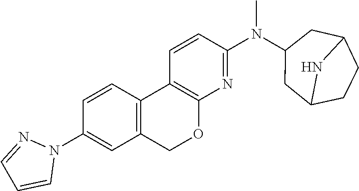 [SMILES: CN(c1ccc2c(n1)OCc1cc(-n3cccn3)ccc1-2)C1CC2CCC(C1)N2] |
| 385 | 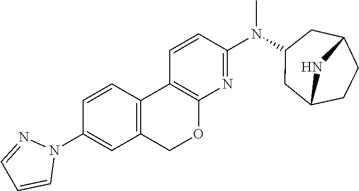 |
| 386 | 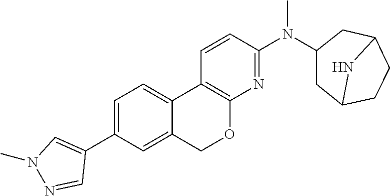 [SMILES: CN(c1ccc2c(n1)OCc1cc(-c3cnn(C)c3)ccc1-2)C1CC2CCC(C1)N2] |
| 387 | 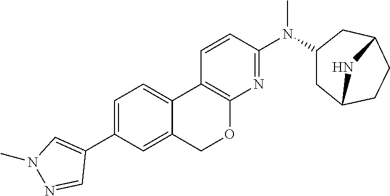 |
| 388 | 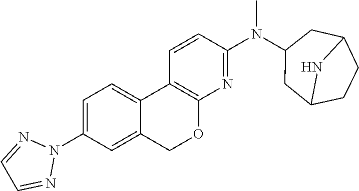 [SMILES: CN(c1ccc2c(n1)OCc1cc(-n3nccn3)ccc1-2)C1CC2CCC(C1)N2] |
| 389 | 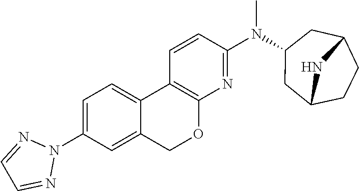 [SMILES: CN(c1ccc2c(n1)OCc1cc(-n3nccn3)ccc1-2)[C@H]1C[C@H]2CC[C@@H](C1)N2] |
| 390 | 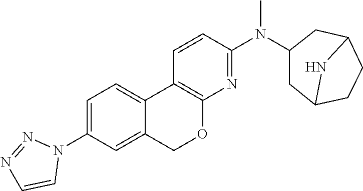 [SMILES: CN(c1ccc2c(n1)OCc1cc(-n3ccnn3)ccc1-2)C1CC2CCC(C1)N2] |
| 391 | 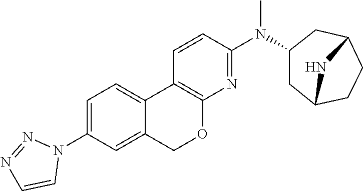 [SMILES: CN(c1ccc2c(n1)OCc1cc(-n3ccnn3)ccc1-2)[C@H]1C[C@H]2CC[C@@H](C1)N2] |
| 392 | 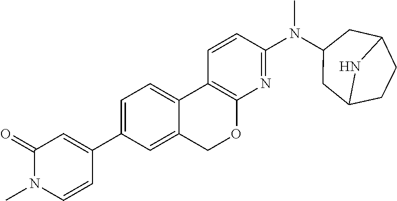 [SMILES: CN(c1ccc2c(n1)OCc1cc(-c3ccn(C)c(=O)c3)ccc1-2)C1CC2CCC(C1)N2] |
| 393 | 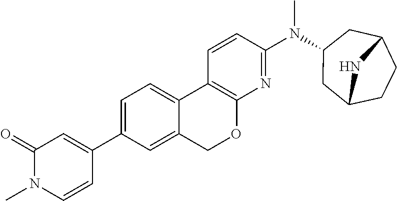 [SMILES: CN(c1ccc2c(n1)OCc1cc(-c3ccn(C)c(=O)c3)ccc1-2)[C@H]1C[C@H]2CC[C@@H](C1)N2] |
| 394 | 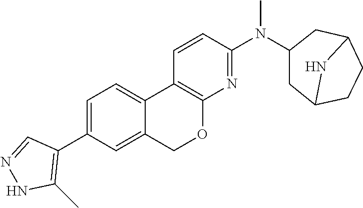 [SMILES: Cc1[nH]ncc1-c1ccc2c(c1)COc1nc(N(C)C3CC4CCC(C3)N4)ccc1-2] |
| 395 | 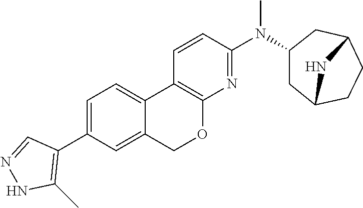 [SMILES: Cc1[nH]ncc1-c1ccc2c(c1)COc1nc(N(C)[C@H]3C[C@H]4CC[C@@H](C3)N4)ccc1-2] |
| 396 | 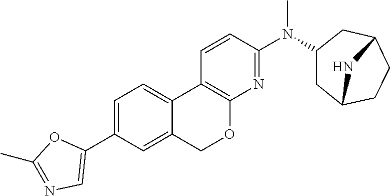 |
| 397 | 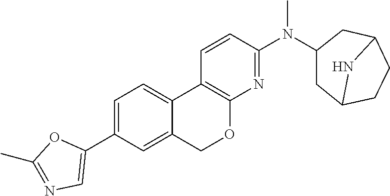 [SMILES: Cc1ncc(-c2ccc3c(c2)COc2nc(N(C)C4CC5CCC(C4)N5)ccc2-3)o1] |
| 398 | 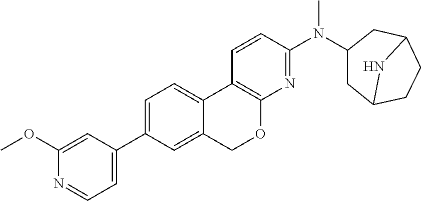 [SMILES: COc1cc(-c2ccc3c(c2)COc2nc(N(C)C4CC5CCC(C4)N5)ccc2-3)ccn1] |
| 399 | 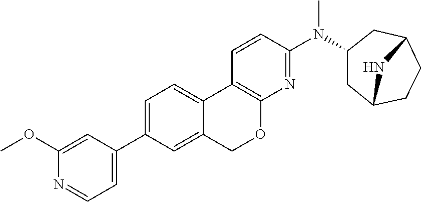 [SMILES: COc1cc(-c2ccc3c(c2)COc2nc(N(C)[C@H]4C[C@H]5CC[C@@H](C4)N5)ccc2-3)ccn1] |
| 400 | 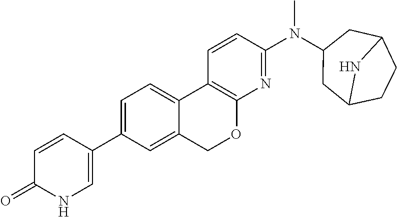 [SMILES: CN(c1ccc2c(n1)OCc1cc(-c3ccc(=O)[nH]c3)ccc1-2)C1CC2CCC(C1)N2] |
| 401 | 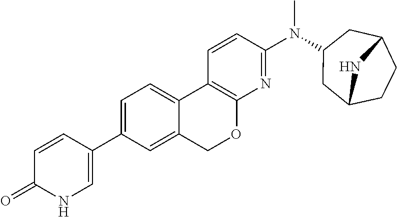 |
| 402 | 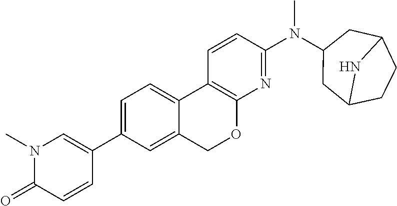 [SMILES: CN(c1ccc2c(n1)OCc1cc(-c3ccc(=O)n(C)c3)ccc1-2)C1CC2CCC(C1)N2] |
| 403 | 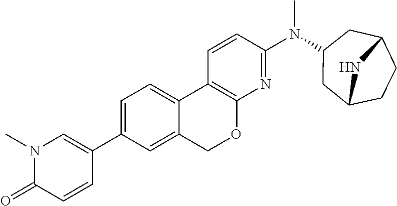 |
| 404 | 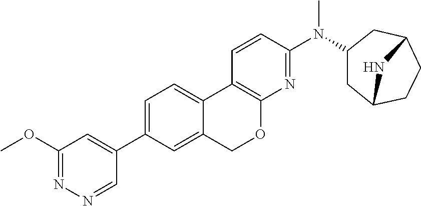 |
| 405 | 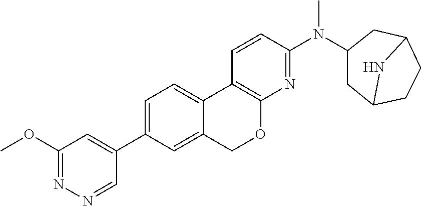 [SMILES: COc1cc(-c2ccc3c(c2)COc2nc(N(C)C4CC5CCC(C4)N5)ccc2-3)cnn1] |
| 406 | 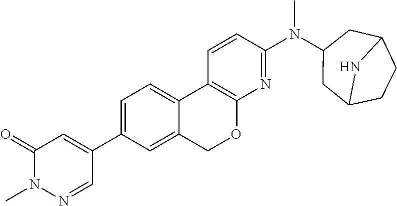 [SMILES: CN(c1ccc2c(n1)OCc1cc(-c3cnn(C)c(=O)c3)ccc1-2)C1CC2CCC(C1)N2] |
| 407 | 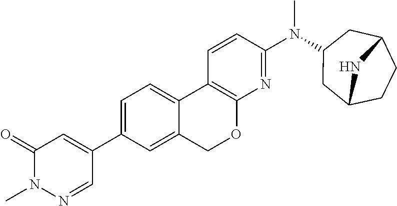 |
| 408 | 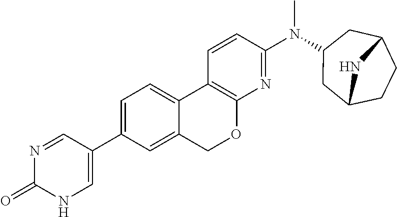 |
| 409 | 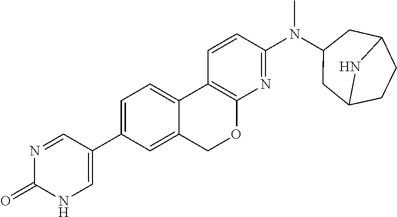 [SMILES: CN(c1ccc2c(n1)OCc1cc(-c3cnc(=O)[nH]c3)ccc1-2)C1CC2CCC(C1)N2] |
| 410 | 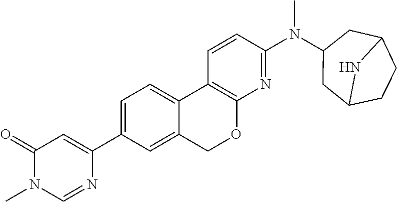 [SMILES: CN(c1ccc2c(n1)OCc1cc(-c3cc(=O)n(C)cn3)ccc1-2)C1CC2CCC(C1)N2] |
| 411 | 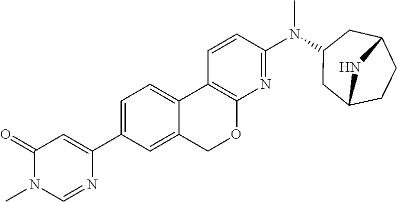 |
| 412 | 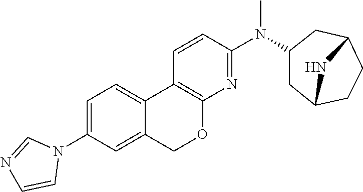 |
| 413 | 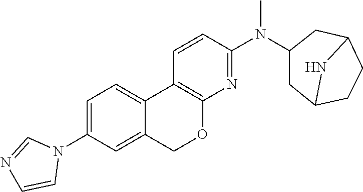 [SMILES: CN(c1ccc2c(n1)OCc1cc(-n3ccnc3)ccc1-2)C1CC2CCC(C1)N2] |
| 414 | 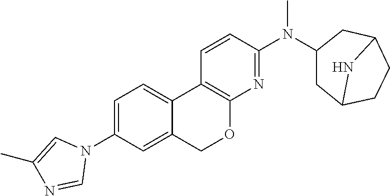 [SMILES: Cc1cn(-c2ccc3c(c2)COc2nc(N(C)C4CC5CCC(C4)N5)ccc2-3)cn1] |
| 415 | 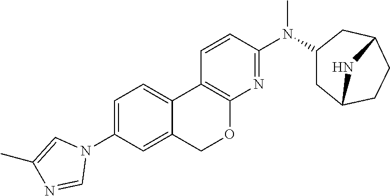 |
| 416 | 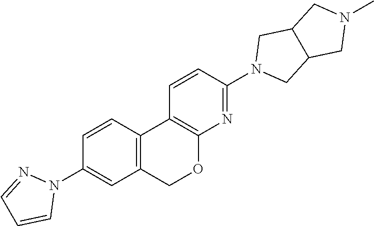 [SMILES: CN1CC2CN(c3ccc4c(n3)OCc3cc(-n5cccn5)ccc3-4)CC2C1] |
| 417 | 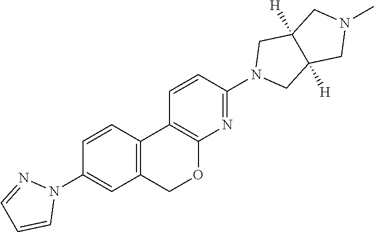 [SMILES: CN1C[C@@H]2CN(c3ccc4c(n3)OCc3cc(-n5cccn5)ccc3-4)C[C@@H]2C1] |
| 418 | 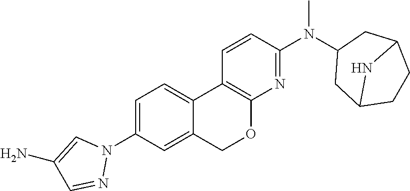 [SMILES: CN(c1ccc2c(n1)OCc1cc(-n3cc(N)cn3)ccc1-2)C1CC2CCC(C1)N2] |
| 419 | 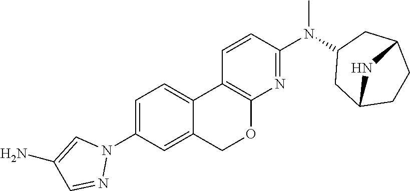 |
| 420 | 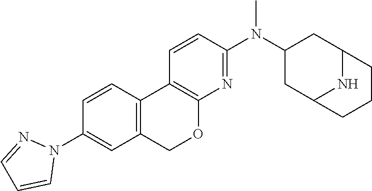 [SMILES: CN(c1ccc2c(n1)OCc1cc(-n3cccn3)ccc1-2)C1CC2CCCC(C1)N2] |
| 421 | 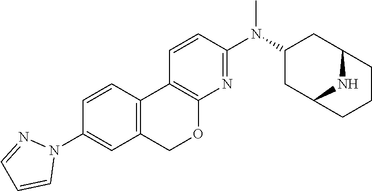 |
| 422 | 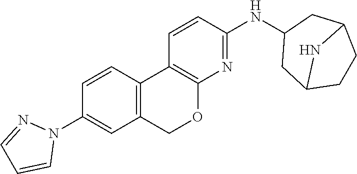 [SMILES: c1cnn(-c2ccc3c(c2)COc2nc(NC4CC5CCC(C4)N5)ccc2-3)c1] |
| 423 | 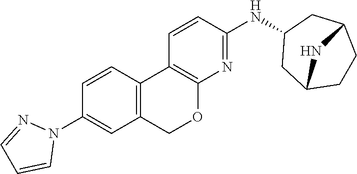 [SMILES: c1cnn(-c2ccc3c(c2)COc2nc(N[C@H]4C[C@H]5CC[C@@H](C4)N5)ccc2-3)c1] |
| 424 | 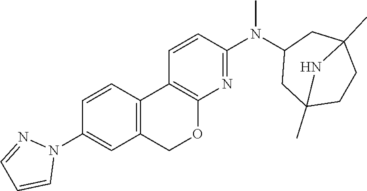 [SMILES: CN(c1ccc2c(n1)OCc1cc(-n3cccn3)ccc1-2)C1CC2(C)CCC(C)(C1)N2] |
| 425 | 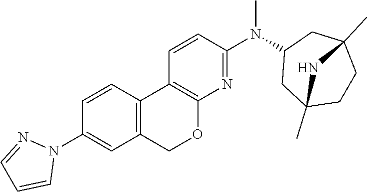 |
| 426 | 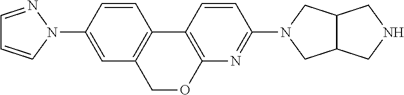 [SMILES: c1cnn(-c2ccc3c(c2)COc2nc(N4CC5CNCC5C4)ccc2-3)c1] |
| 427 | 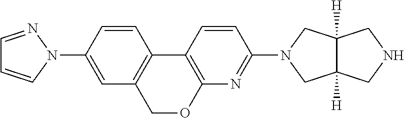 [SMILES: c1cnn(-c2ccc3c(c2)COc2nc(N4C[C@H]5CNC[C@H]5C4)ccc2-3)c1] |
| 428 | 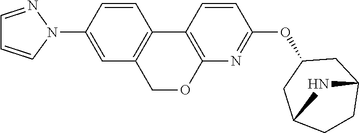 [SMILES: c1cnn(-c2ccc3c(c2)COc2nc(O[C@H]4C[C@H]5CC[C@@H](C4)N5)ccc2-3)c1] |
| 429 | 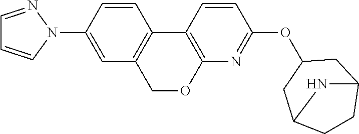 [SMILES: c1cnn(-c2ccc3c(c2)COc2nc(OC4CC5CCC(C4)N5)ccc2-3)c1] |
| 430 | 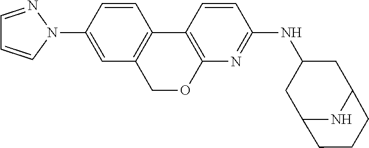 [SMILES: c1cnn(-c2ccc3c(c2)COc2nc(NC4CC5CCCC(C4)N5)ccc2-3)c1] |
| 431 | 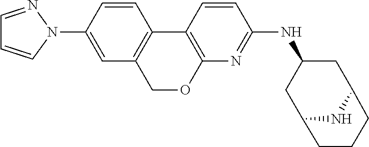 [SMILES: c1cnn(-c2ccc3c(c2)COc2nc(N[C@H]4C[C@H]5CCC[C@@H](C4)N5)ccc2-3)c1] |
| 432 | 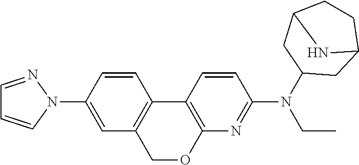 [SMILES: CCN(c1ccc2c(n1)OCc1cc(-n3cccn3)ccc1-2)C1CC2CCC(C1)N2] |
| 433 | 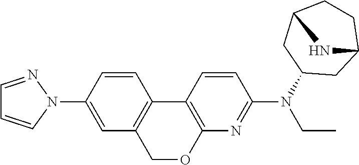 [SMILES: CCN(c1ccc2c(n1)OCc1cc(-n3cccn3)ccc1-2)[C@H]1C[C@H]2CC[C@@H](C1)N2] |
| 434 | 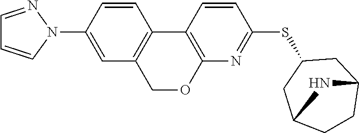 [SMILES: c1cnn(-c2ccc3c(c2)COc2nc(S[C@H]4C[C@H]5CC[C@@H](C4)N5)ccc2-3)c1] |
| 435 | 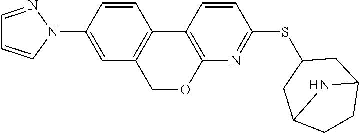 [SMILES: c1cnn(-c2ccc3c(c2)COc2nc(SC4CC5CCC(C4)N5)ccc2-3)c1] |
| 436 | 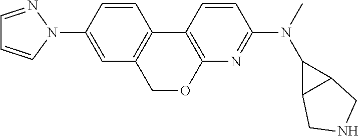 [SMILES: CN(c1ccc2c(n1)OCc1cc(-n3cccn3)ccc1-2)C1C2CNCC21] |
| 437 | 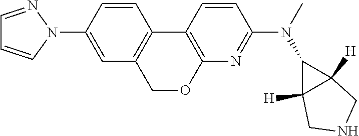 [SMILES: CN(c1ccc2c(n1)OCc1cc(-n3cccn3)ccc1-2)[C@@H]1[C@@H]2CNC[C@@H]21] |
| 438 | 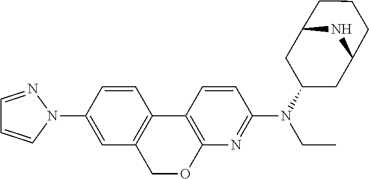 [SMILES: CCN(c1ccc2c(n1)OCc1cc(-n3cccn3)ccc1-2)[C@H]1C[C@H]2CCC[C@@H](C1)N2] |
| 439 | 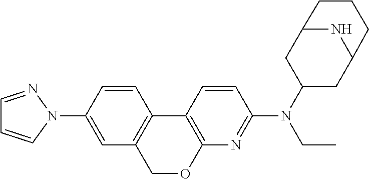 [SMILES: CCN(c1ccc2c(n1)OCc1cc(-n3cccn3)ccc1-2)C1CC2CCCC(C1)N2] |
| 440 | 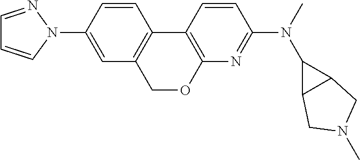 [SMILES: CN1CC2C(C1)C2N(C)c1ccc2c(n1)OCc1cc(-n3cccn3)ccc1-2] |
| 441 | 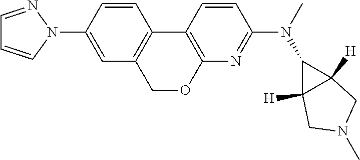 [SMILES: CN1C[C@@H]2[C@H](C1)[C@@H]2N(C)c1ccc2c(n1)OCc1cc(-n3cccn3)ccc1-2] |
| 442 | 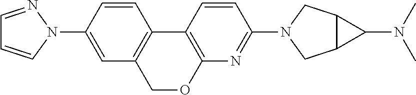 [SMILES: CN(C)C1C2CN(c3ccc4c(n3)OCc3cc(-n5cccn5)ccc3-4)CC21] |
| 443 | 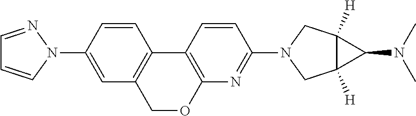 [SMILES: CN(C)[C@@H]1[C@@H]2CN(c3ccc4c(n3)OCc3cc(-n5cccn5)ccc3-4)C[C@@H]21] |
| 444 | 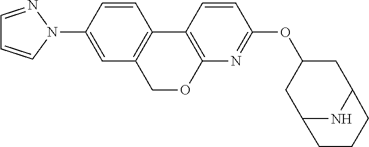 [SMILES: c1cnn(-c2ccc3c(c2)COc2nc(OC4CC5CCCC(C4)N5)ccc2-3)c1] |
| 445 | 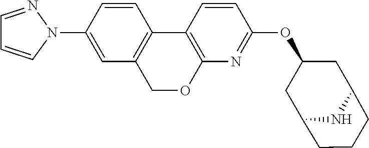 [SMILES: c1cnn(-c2ccc3c(c2)COc2nc(O[C@H]4C[C@H]5CCC[C@@H](C4)N5)ccc2-3)c1] |
| 446 | 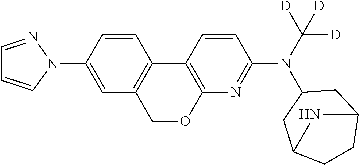 [SMILES: [2H]C([2H])([2H])N(c1ccc2c(n1)OCc1cc(-n3cccn3)ccc1-2)C1CC2CCC(C1)N2] |
| 447 | 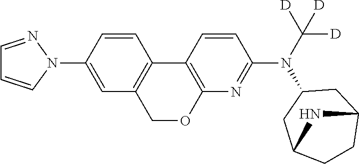 [SMILES: [2H]C([2H])([2H])N(c1ccc2c(n1)OCc1cc(-n3cccn3)ccc1-2)[C@H]1C[C@H]2CC[C@@H](C1)N2] |
| 448 | 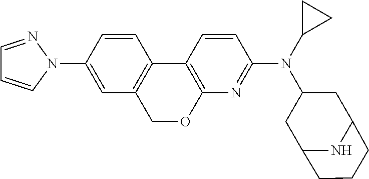 [SMILES: c1cnn(-c2ccc3c(c2)COc2nc(N(C4CC4)C4CC5CCCC(C4)N5)ccc2-3)c1] |
| 449 | 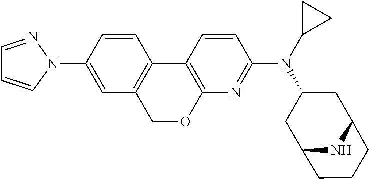 [SMILES: c1cnn(-c2ccc3c(c2)COc2nc(N(C4CC4)[C@H]4C[C@H]5CCC[C@@H](C4)N5)ccc2-3)c1] |
| 450 | 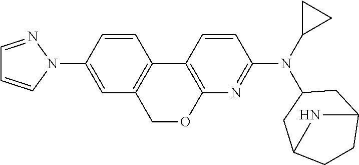 [SMILES: c1cnn(-c2ccc3c(c2)COc2nc(N(C4CC4)C4CC5CCC(C4)N5)ccc2-3)c1] |
| 451 | 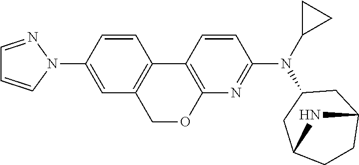 [SMILES: c1cnn(-c2ccc3c(c2)COc2nc(N(C4CC4)[C@H]4C[C@H]5CC[C@@H](C4)N5)ccc2-3)c1] |
| 452 | 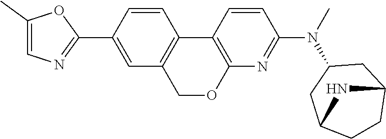 [SMILES: Cc1cnc(-c2ccc3c(c2)COc2nc(N(C)[C@H]4C[C@H]5CC[C@@H](C4)N5)ccc2-3)o1] |
| 453 | 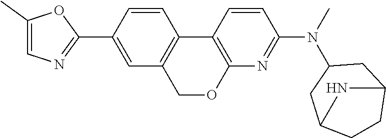 [SMILES: Cc1cnc(-c2ccc3c(c2)COc2nc(N(C)C4CC5CCC(C4)N5)ccc2-3)o1] |
| 454 | 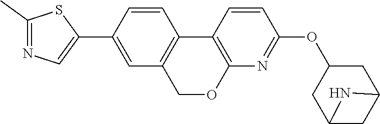 [SMILES: Cc1ncc(-c2ccc3c(c2)COc2nc(OC4CC5CC(C4)N5)ccc2-3)s1] |
| 455 | 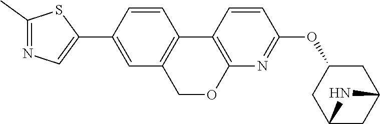 [SMILES: Cc1ncc(-c2ccc3c(c2)COc2nc(O[C@H]4C[C@H]5C[C@@H](C4)N5)ccc2-3)s1] |
| 456 | 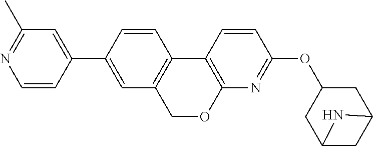 [SMILES: Cc1cc(-c2ccc3c(c2)COc2nc(OC4CC5CC(C4)N5)ccc2-3)ccn1] |
| 457 | 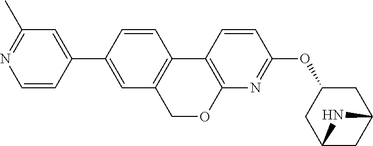 [SMILES: Cc1cc(-c2ccc3c(c2)COc2nc(O[C@H]4C[C@H]5C[C@@H](C4)N5)ccc2-3)ccn1] |
| 458 | 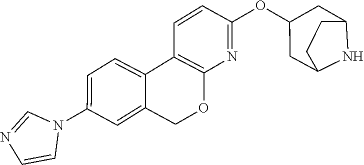 [SMILES: c1cn(-c2ccc3c(c2)COc2nc(OC4CC5CCC(C4)N5)ccc2-3)cn1] |
| 459 | 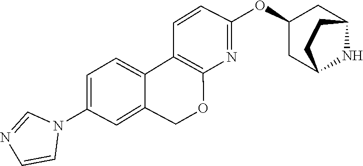 |
| 460 | 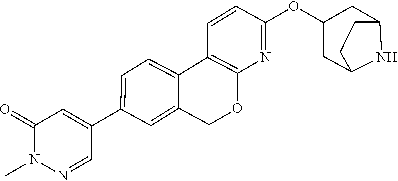 [SMILES: Cn1ncc(-c2ccc3c(c2)COc2nc(OC4CC5CCC(C4)N5)ccc2-3)cc1=O] |
| 461 | 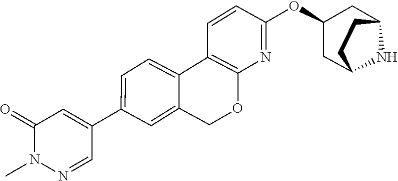 |
| 462 | 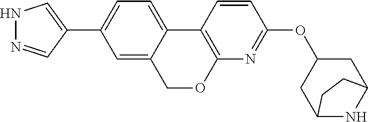 [SMILES: c1cc2c(cc1-c1cn[nH]c1)COc1nc(OC3CC4CCC(C3)N4)ccc1-2] |
| 463 | 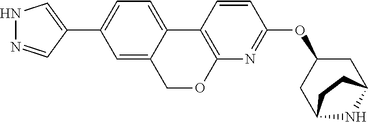 |
| 464 | 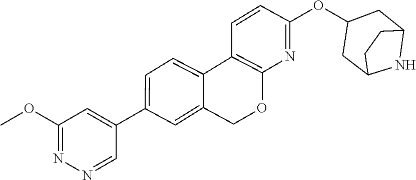 [SMILES: COc1cc(-c2ccc3c(c2)COc2nc(OC4CC5CCC(C4)N5)ccc2-3)cnn1] |
| 465 | 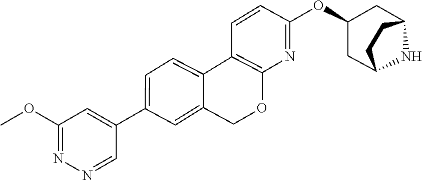 |
| 466 | 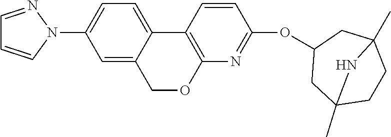 [SMILES: CC12CCC(C)(CC(Oc3ccc4c(n3)OCc3cc(-n5cccn5)ccc3-4)C1)N2] |
| 467 | 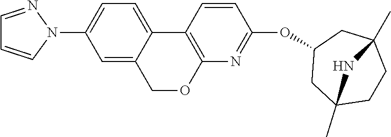 [SMILES: C[C@]12CC[C@](C)(C[C@H](Oc3ccc4c(n3)OCc3cc(-n5cccn5)ccc3-4)C1)N2] |
| 468 | 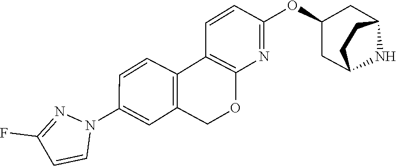 |
| 469 | 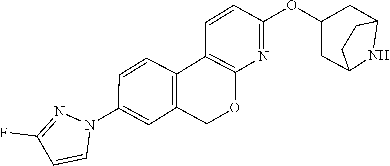 [SMILES: Fc1ccn(-c2ccc3c(c2)COc2nc(OC4CC5CCC(C4)N5)ccc2-3)n1] |
| 470 | 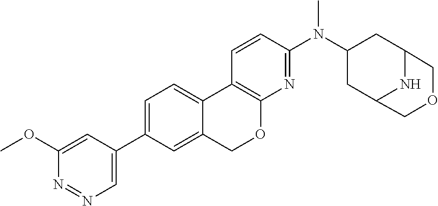 [SMILES: COc1cc(-c2ccc3c(c2)COc2nc(N(C)C4CC5COCC(C4)N5)ccc2-3)cnn1] |
| 471 | 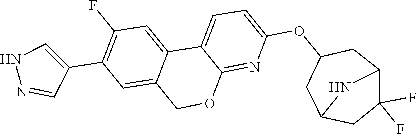 [SMILES: Fc1cc2c(cc1-c1cn[nH]c1)COc1nc(OC3CC4CC(F)(F)C(C3)N4)ccc1-2] |
| 472 | 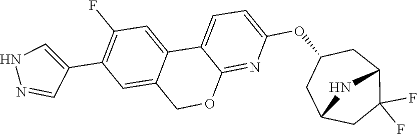 [SMILES: Fc1cc2c(cc1-c1cn[nH]c1)COc1nc(O[C@H]3C[C@H]4CC(F)(F)[C@@H](C3)N4)ccc1-2] |
| 473 | 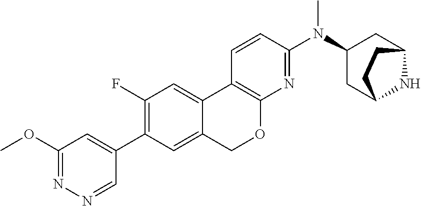 |
| 474 | 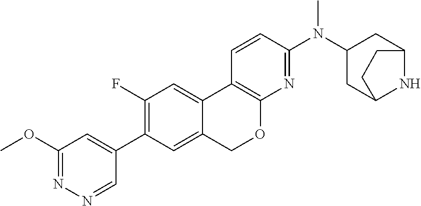 [SMILES: COc1cc(-c2cc3c(cc2F)-c2ccc(N(C)C4CC5CCC(C4)N5)nc2OC3)cnn1] |
| 475 | 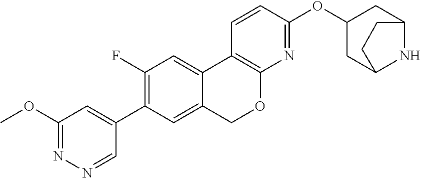 [SMILES: COc1cc(-c2cc3c(cc2F)-c2ccc(OC4CC5CCC(C4)N5)nc2OC3)cnn1] |
| 476 | 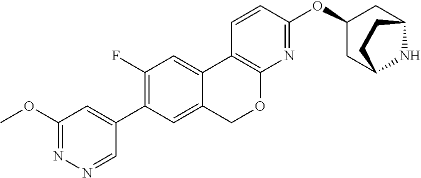 |
| 477 | 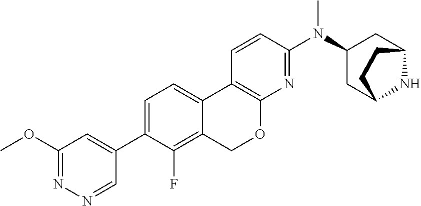 |
| 478 | 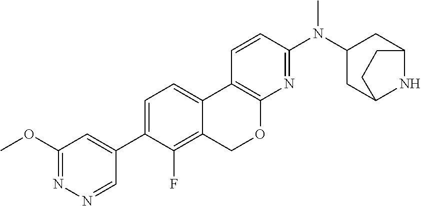 [SMILES: COc1cc(-c2ccc3c(c2F)COc2nc(N(C)C4CC5CCC(C4)N5)ccc2-3)cnn1] |
| 479 | 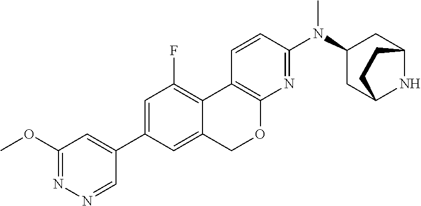 |
| 480 | 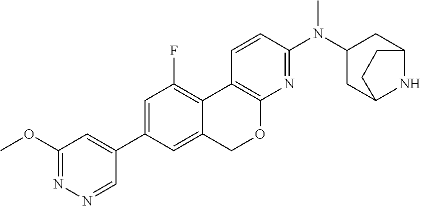 [SMILES: COc1cc(-c2cc(F)c3c(c2)COc2nc(N(C)C4CC5CCC(C4)N5)ccc2-3)cnn1] |
| 481 | 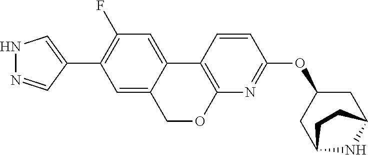 |
| 482 | 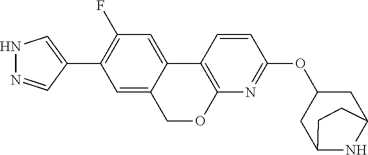 [SMILES: Fc1cc2c(cc1-c1cn[nH]c1)COc1nc(OC3CC4CCC(C3)N4)ccc1-2] |
| 483 | 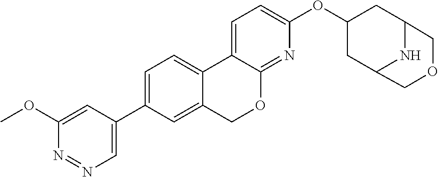 [SMILES: COc1cc(-c2ccc3c(c2)COc2nc(OC4CC5COCC(C4)N5)ccc2-3)cnn1] |
| 484 | 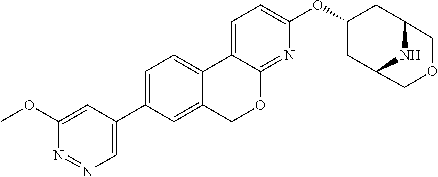 [SMILES: COc1cc(-c2ccc3c(c2)COc2nc(O[C@H]4C[C@H]5COC[C@@H](C4)N5)ccc2-3)cnn1] |
| 485 | 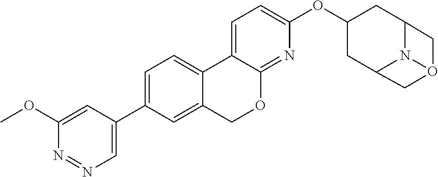 |
| 486 | 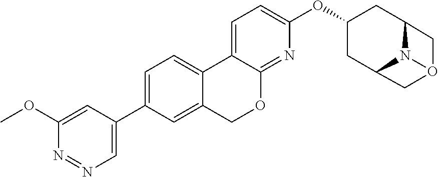 |
| 487 | 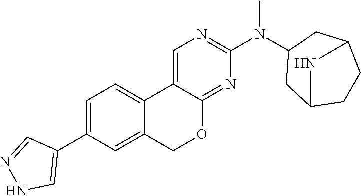 [SMILES: CN(c1ncc2c(n1)OCc1cc(-c3cn[nH]c3)ccc1-2)C1CC2CCC(C1)N2] |
| 488 | 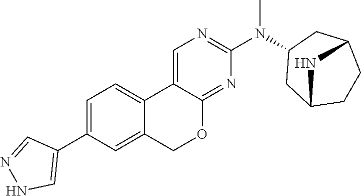 |
| 489 | 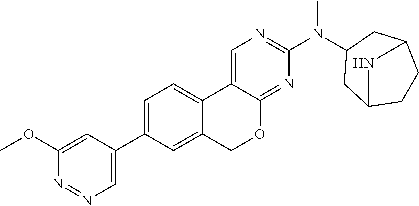 [SMILES: COc1cc(-c2ccc3c(c2)COc2nc(N(C)C4CC5CCC(C4)N5)ncc2-3)cnn1] |
| 490 | 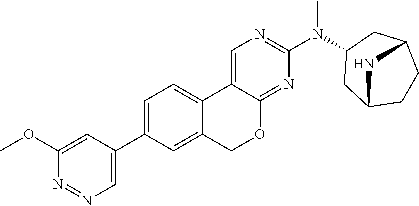 [SMILES: COc1cc(-c2ccc3c(c2)COc2nc(N(C)[C@H]4C[C@H]5CC[C@@H](C4)N5)ncc2-3)cnn1] |
| 491 | 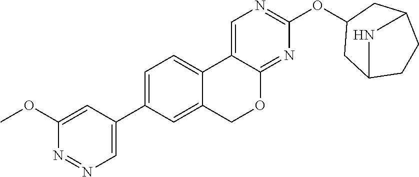 [SMILES: COc1cc(-c2ccc3c(c2)COc2nc(OC4CC5CCC(C4)N5)ncc2-3)cnn1] |
| 492 | 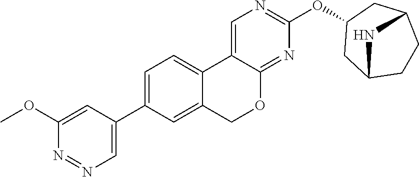 [SMILES: COc1cc(-c2ccc3c(c2)COc2nc(O[C@H]4C[C@H]5CC[C@@H](C4)N5)ncc2-3)cnn1] |
| 493 | 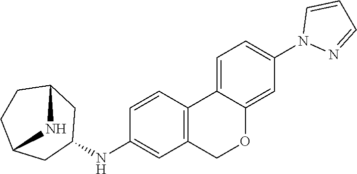 [SMILES: c1cnn(-c2ccc3c(c2)OCc2cc(N[C@H]4C[C@H]5CC[C@@H](C4)N5)ccc2-3)c1] |
| 494 | 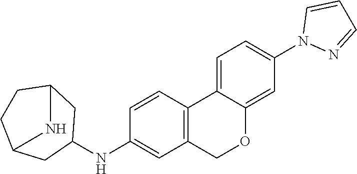 [SMILES: c1cnn(-c2ccc3c(c2)OCc2cc(NC4CC5CCC(C4)N5)ccc2-3)c1] |
| 495 | 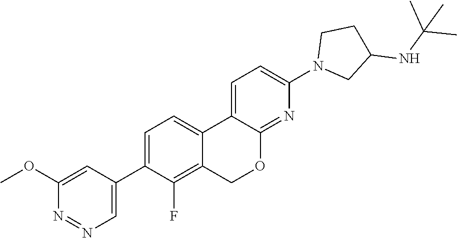 [SMILES: COc1cc(-c2ccc3c(c2F)COc2nc(N4CCC(NC(C)(C)C)C4)ccc2-3)cnn1] |
| 496 | 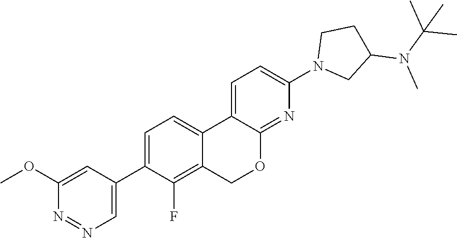 [SMILES: COc1cc(-c2ccc3c(c2F)COc2nc(N4CCC(N(C)C(C)(C)C)C4)ccc2-3)cnn1] |

In some embodiments, for Formula (I), A is monocyclic heterocyclyl (e.g., 2-methyl piperidinyl); B is monocyclic heteroaryl (e.g., pyrazolyl); L is —N(R⁴)— (e.g., NMe); Z¹, Z², Z³, Z⁴, Z⁵, and Z⁶ are each independently C(R⁶) (e.g., CH); X is O; Y is C(R^{7a})(R^{7b}) (e.g., CH₂); n and m are both 1; and R^{2a} and R^{2b} are each independently hydrogen. In some embodiments, the compound of Formulas (I), (I-a), (I-c), and (I-d) is Compound 100, 101, 102, 336, 337, or a pharmaceutically acceptable salt, solvate, hydrate, tautomer, or stereoisomer thereof.

In some embodiments, for Formula (I), A is monocyclic heterocyclyl (e.g., 2-methyl piperidinyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is C(R^{7a})(R^{7b}) (e.g., CH₂); Y is O; n and m are both 1; and R^{2a} and R^{2b} are each independently hydrogen. In some embodiments, the compound of Formulas (I), (I-b), (I-c), and (I-e) is Compound 103, 117, 118, 119, 130, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is O; Y is C(R^{7a})(R^{7b}) (e.g., CH₂); n and m are both 1; and R^{2a} and R^{2b} are each independently hydrogen. In some embodiments, the compound of Formulas (I), (I-a), (I-c), and (I-d) is Compound 104, 338, 339, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is C(R^{7a})(R^{7b}) (e.g., CH₂); Y is O; n and m are both 1; and R^{2a} and R^{2b} are each independently hydrogen. In some embodiments, the compound of Formulas (I), (I-b), (I-c), and (I-e) is Compound 105, 340, 341, or a pharmaceutically acceptable salt, solvate, hydrate, tautomer, or stereoisomer thereof.

In some embodiments, for Formula (I), A is monocyclic heterocyclyl (e.g., 2-methylpiperidinyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); Z² is N; X is O; Y is C(R^{7a})(R^{7b}) (e.g., CH₂); n and m are both 1; and R^{2a} and R^{2b} are each independently hydrogen. In some embodiments, the compound of Formulas (I), (I-a), (I-c), and (I-d) is Compound 106, or a pharmaceutically acceptable salt, solvate, hydrate, tautomer, or stereoisomer thereof.

In some embodiments, for Formula (I), A is monocyclic heterocyclyl (e.g., 2-methyl piperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is C(R^{7a})(R^{7b}) (e.g., CH₂); Y is O; n and m are both 1; and R^{2a} and R^{2b} are each independently hydrogen. In some embodiments, the compound of Formulas (I), (I-b), (I-c), and (I-e) is Compound 107, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., Nme); Z¹, Z³, and Z⁴ are each independently C(R⁶) (e.g., CH); Z² is N; X is O; Y is C(R^{7a})(R^{7b}) (e.g., CH₂); n and m are both 1; and R^{2a} and R^{2b} are each independently hydrogen. In some embodiments, the compound of Formulas (I), (I-a), (I-c), and (I-d) is Compound 108, 342, 343, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., Nme); Z¹, Z³, Z⁴, Z⁵, and Z⁶ are each are each independently C(R⁶) (e.g., CH); Z² is N; X is C(R^{7a})(R^{7b}) (e.g., CH₂); Y is O; n and m are both 1; and R^{2a} and R^{2b} are each independently hydrogen. In some embodiments, the compound of Formulas (I), (I-b), (I-c), and (I-e) is Compound 109, 344, 345, or a pharmaceutically acceptable salt, solvate, hydrate, tautomer, or stereoisomer thereof.

In some embodiments, for Formula (I), A is monocyclic heterocyclyl (e.g., 2-methyl piperidinyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is O; Y is C(R^{7a})(R^{7b}) (e.g., CH₂); n is 1; m is 2; and R^{2a} and R^{2b} are each independently hydrogen. In some embodiments, the compound of Formulas (I), (I-a), (I-c), and (I-d) is Compound 110, or a pharmaceutically acceptable salt, solvate, hydrate, tautomer, or stereoisomer thereof.

In some embodiments, for Formula (I), A is monocyclic heterocyclyl (e.g., 2-methyl piperidinyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is O; Y is C(R^{7a})(R^{7b}) (e.g., CH₂); n is 1; m is 2; and R^{2a} and R^{2b} are each independently hydrogen. In some embodiments, the compound of Formulas (I), (I-a), (I-c), and (I-d) is Compound 111, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is C(R^{7a})(R^{7b}) (e.g., CH₂); Y is O; n is 2; m is 1; and R^{2a} and R^{2b} are each independently hydrogen. In some embodiments, the compound of Formulas (I), (I-b), (I-c), and (I-e) is Compound 112, 346, 347, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., Nme); Z¹, Z², Z³, Z⁴, Z⁵, and Z⁶ are each are each independently C(R⁶) (e.g., CH); X is C(R^{7a})(R^{7b}) (e.g., CH₂); Y is O; n is 2; m is 1; and R^{2a} and R^{2b} are each independently hydrogen. In some embodiments, the compound of Formulas (I), (I-b), (I-c), and (I-e) is Compound 113, 348, 349, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; m is 1; and R^{2a} and R^{2b} are each independently hydrogen. In some embodiments, the compound of Formulas (I), (I-b), (I-c), and (I-e) is Compound 114, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is N(R^{7c}) (e.g., Nme); Y is C(R^{7a})(R^{7b}) (e.g., CH₂); n is 1; m is 2; and R^{2a} and R^{2b} are each independently hydrogen. In some embodiments, the compound of Formulas (I), (I-a), (I-c), and (I-f) is Compound 115, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 116, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 117, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., NH); Z¹, Z², Z³, Z⁴, Z⁵, and Z⁶ are each independently C(R⁶) (e.g., CH); X is C(R^{7a})(R^{7b}) (e.g., CH₂); Y is 0; n is 1; and m is 1. In some embodiments, the compound of Formulas (I), (I-a), (I-b), (I-d), (I-e), (I-g), and (I-i) is Compound 118, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., NH); Z¹, Z², Z³, Z⁴, Z⁵, and Z⁶ are each independently C(R⁶) (e.g., CH); X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 119, 350, 351, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 121, 124, 352, 353, 358, 359, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 122, 125, 354, 355, 360, 361, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —O—; Z¹, Z³, Z⁴, Z⁵, and Z⁶ are each independently C(R⁶) (e.g., CH); Z² is N; X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 123, 356, 357, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 127, 128, 362, 363, 364, 365, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 129, 366, 367, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 130, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z², Z³, Z⁴, Z⁵, and Z⁶ are each independently C(R⁶) (e.g., CH); X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 131, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., NH); Z¹, Z², Z³, Z⁴, Z⁵, and Z⁶ are each independently C(R⁶) (e.g., CH); X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 132, 139, 368, 369, 374, 375, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., NH); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 134, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., NH); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 1; and m is 1. In some embodiments, the compound of Formulas (I), (I-a), (I-b), (I-d), (I-e), (I-g), and (I-i) is Compound 135, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., NH); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 136, 370, 371, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., NH); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 137, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, and Z⁶ are each independently C(R⁶) (e.g., CH); Z² and Z⁵ are N; X is C(R^{7a})(R^{7b}) (e.g., CH₂); Y is O; n is 1; and m is 1. In some embodiments, the compound of Formulas (I) and (I-a) is Compound 138, 372, 373, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., NH); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 140, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., NH); Z¹, Z³, Z⁴, Z⁵, and Z⁶ are each independently C(R⁶) (e.g., CH); Z² is N; X is C(R^{7a})(R^{7b}) (e.g., CH₂); Y is O; n is 2; and m is 1. In some embodiments, the compound of Formulas (I), (I-a), (I-b), (I-d), (I-e), (I-g), and (I-i) is Compound 141, 376, 377, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, and Z⁶ are each independently C(R⁶) (e.g., CH); Z² and Z⁵ are N; X is O; Y is C(R^{7a})(R^{7b}) (e.g., CH₂); n is 1; and m is 1. In some embodiments, the compound of Formulas (I) and (I-a) is Compound 142, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., NH); Z¹, Z³, Z⁴, and Z⁶ are each independently C(R⁶) (e.g., CH); Z² and Z⁵ are N; X is O; Y is C(R^{7a})(R^{7b}) (e.g., CH₂); n is 1; and m is 1. In some embodiments, the compound of Formulas (I) and (I-a) is Compound 143, 378, 379, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z³, Z⁴, and Z⁶ are each independently C(R⁶) (e.g., CH); Z² and Z⁵ are N; X is O; Y is C(R^{7a})(R^{7b}) (e.g., CH₂); n is 1; and m is 1. In some embodiments, the compound of Formulas (I) and (I-a) is Compound 144, 380, 381, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is O; Y is C(R^{7a})(R^{7b}) (e.g., CH₂); n is 1; and m is 2. In some embodiments, the compound of Formulas (I), (I-a), (I-b), (I-c), (I-e), (I-f), and (I-i) is Compound 145, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z³, Z⁴, Z⁵, and Z⁶ are each independently C(R⁶) (e.g., CH); Z² is N; X is O; Y is C(R^{7a})(R^{7b}) (e.g., CH₂); n is 1; and m is 2. In some embodiments, the compound of Formulas (I), (I-a), (I-b), (I-c), (I-e), (I-f), and (I-i) is Compound 146, 382, 383, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L is —N(R⁴)— (e.g., Nme); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 147, 148, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L is —N(R⁴)— (e.g., Nme); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 149, 384, 385, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-methyl-1H-pyrazolyl); L is —N(R⁴)— (e.g., Nme); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 150, 386, 387, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2H-1,2,3-triazolyl); L is —N(R⁴)— (e.g., Nme); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 151, 388, 389, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-1,2,3-triazolyl); L is —N(R⁴)— (e.g., Nme); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 152, 390, 391, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-methylpyridin-2 (1H)—I); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 153, 392, 393, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 5-methyl-1H-pyrazolyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 154, 394, 395, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methyloxazolyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 155, 396, 397, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methoxypyridinyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 156, 398, 399, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyridin-2 (1H)-onyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 157, 400, 401, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1-methylpyridinyl)-onyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 158, 402, 403, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-methoxypyridazinyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 159, 404, 405, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methylpyridazin-3 (2H)-onyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 160, 406, 407, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrimidin-2 (1H)—I); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 161, 408, 409, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 3-methylpyrimidin-4 (3H)-onyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 162, 410, 411, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-imidazolyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 163, 412, 413, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 4-methyl-1H-imidazolyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 164, 414, 415, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —N(R⁴)— (e.g., NH); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 165, or a pharmaceutically acceptable salt, solvate, hydrate, tautomer, or stereoisomer thereof.

In some embodiments, for Formula (I), A is monocyclic heterocyclyl (e.g., azetidinyl); B is monocyclic heteroaryl (e.g., 1H-pyrazolyl); L is —N(R⁴)— (e.g., NH); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 166, or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 167, or a pharmaceutically acceptable salt, solvate, hydrate, tautomer, or stereoisomer thereof.

In some embodiments, for Formula (I), A is bicyclic heterocyclyl (e.g., 2,6-diazaspiro[3.5]nonanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 168, or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 169, or a pharmaceutically acceptable salt, solvate, hydrate, tautomer, or stereoisomer thereof.

In some embodiments, for Formula (I), A is bicyclic heterocyclyl (e.g., 2,7-diazaspiro[3.5]nonanyl); B is monocyclic heteroaryl (e.g., 2-methyloctahydropyrrolo[3,4-c]pyrrolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 170, 416, 417, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is 0; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 171, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 4-amino-1H-pyrazolyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 172, 418, 419, or a pharmaceutically acceptable salt, solvate, hydrate, tautomer, or stereoisomer thereof.

In some embodiments, for Formula (I), A is monocyclic heterocyclyl (e.g., piperazinyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g) and (I-i) is Compound 173, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1H-pyrazolyl); L is —N(R⁴)— (e.g., NH); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 174, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1H-pyrazolyl); L is —N(R⁴)— (e.g., NMe); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 175, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1H-pyrazolyl); L is —N(R⁴)— (e.g., NCD₃); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I) and (I-i) is Compound 176, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1H-pyrazolyl); L is —N(R⁴)— (e.g., Net); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 177, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., 1H-pyrazolyl); L is —N(R⁴)— (e.g., Nme); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), and (I-i) is Compound 178, 420, 421, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-pyrazolyl); L is —N(R⁴)— (e.g., NH); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), and (I-i) is Compound 179, 422, 423, or a pharmaceutically acceptable salt, solvate, hydrate, tautomer, or stereoisomer thereof.

In some embodiments, for Formula (I), A is bicyclic heterocyclyl (e.g., 1,5-dimethyl-8-azabicyclo[3.2.1]octanyl); B is monocyclic heteroaryl (e.g., 1H-pyrazolyl); L is —N(R⁴)— (e.g., NH); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), and (I-i) is Compound 180, 424, 425, or a pharmaceutically acceptable salt, solvate, hydrate, tautomer, or stereoisomer thereof.

In some embodiments, for Formula (I), A is bicyclic heterocyclyl (e.g., 2,6-diazaspiro[3.5]nonanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 181, or a pharmaceutically acceptable salt, solvate, hydrate, tautomer, or stereoisomer thereof.

In some embodiments, for Formula (I), A is bicyclic heterocyclyl (e.g., 2,8-diazaspiro[4.5]decanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 182, or a pharmaceutically acceptable salt, solvate, hydrate, tautomer, or stereoisomer thereof.

In some embodiments, for Formula (I), A is bicyclic heterocyclyl (e.g., 2,7-diazaspiro[4.5]decanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 183, or a pharmaceutically acceptable salt, solvate, hydrate, tautomer, or stereoisomer thereof.

In some embodiments, for Formula (I), A is bicyclic heterocyclyl (e.g., 2,8-diazaspiro[4.5]decanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 184, or a pharmaceutically acceptable salt, solvate, hydrate, tautomer, or stereoisomer thereof.

In some embodiments, for Formula (I), A is bicyclic heterocyclyl (e.g., octahydropyrrolo[3,4-c]pyrrolyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 185, 426, 427, or a pharmaceutically acceptable salt, solvate, hydrate, tautomer, or stereoisomer thereof.

In some embodiments, for Formula (I), A is monocyclic heterocyclyl (e.g., 2,2-dimethylpiperidinyl); B is monocyclic heteroaryl (e.g., 1H-pyrazolyl); L is 0; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 186, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1H-pyrazolyl); L is 0; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1.

In some embodiments, the compound of Formulas (I), (I-a), (I-b), (I-c), (I-e), (I-f), and (I-i) is Compound 187, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-pyrazolyl); L is 0; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 188, 428, 429, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —N(R⁴)— (e.g., —N(CH₂CH₃)—); Z¹, Z³, Z⁴, Z⁵ and Z⁶ are each independently C(R⁶) (e.g., CH); Z² is N; X is O; Y is C(R^{7a})(R^{I}) (e.g., CH₂); n is 1; and m is 1. In some embodiments, the compound of Formulas (I), (I-a), (I-b), (I-c), (I-e), (I-f), and (I-i) is Compound 189, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., 1H-pyrazolyl); L is —N(R⁴)— (e.g., —NH—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 190, 430, 431, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-pyrazolyl); L is —N(R⁴)— (e.g., —N(CH₂CH₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 191, 432, 433, or a pharmaceutically acceptable salt, solvate, hydrate, tautomer, or stereoisomer thereof.

In some embodiments, for Formula (I), A is bicyclic heterocyclyl (e.g., 2,7-diazaspiro[4.5]decanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 192, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-pyrazolyl); L is —S—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 193, 434, 435, or a pharmaceutically acceptable salt, solvate, hydrate, tautomer, or stereoisomer thereof.

In some embodiments, for Formula (I), A is bicyclic heterocyclyl (e.g., 3-azabicyclo[3.1.0]hexanyl); B is monocyclic heteroaryl (e.g., 1H-pyrazolyl); L is —N(R⁴)— (e.g., —N(CH₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 194, 436, 437, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., 1H-pyrazolyl); L is —N(R⁴)— (e.g., —N(CH₂CH₃)—); Z¹, Z³, Z⁴, Z⁵ and Z⁶ are each independently C(R⁶) (e.g., CH); Z² is N; X is O; Y is C(R^{7a})(R^{7I}) (e.g., CH₂); n is 1; and m is 1. In some embodiments, the compound of Formulas (I), (I-a), (I-b), (I-c), (I-e), (I-f), and (I-i) is Compound 195, 438, 439, or a pharmaceutically acceptable salt, solvate, hydrate, tautomer, or stereoisomer thereof.

In some embodiments, for Formula (I), A is bicyclic heterocyclyl (e.g., 3-methyl-3-azabicyclo[3.1.0]hexanyl); B is monocyclic heteroaryl (e.g., 1H-pyrazolyl); L is —N(R⁴)— (e.g., —N(CH₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 196, 440, 441, or a pharmaceutically acceptable salt, solvate, hydrate, tautomer, or stereoisomer thereof.

In some embodiments, for Formula (I), A is bicyclic heterocyclyl (e.g., 6-(N,N-dimethyl)amino-3-azabicyclo[3.1.0]hexanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 197, 442, 443, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., 1H-pyrazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 198, 444, 445, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —N(R⁴)— (e.g., —N(CD₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I) and (I-i) is Compound 199, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., 1H-pyrazolyl); L is —N(R⁴)— (e.g., —N(CD₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I) and (I-i) is Compound 200, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-pyrazolyl); L is —N(R⁴)— (e.g., —N(CD₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I) and (I-i) is Compound 201 446, 447, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 1H-pyrazolyl); L is —N(R⁴)— (e.g., —N(cyclopropyl)-); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 202, or a pharmaceutically acceptable salt, solvate, hydrate, tautomer, or stereoisomer thereof.

In some embodiments, for Formula (I), A is bicyclic heterocyclyl (e.g., 9-azabicyclo[3.3.1]nonanyl); B is monocyclic heteroaryl (e.g., 1H-pyrazolyl); L is —N(R⁴)— (e.g., —N(cyclopropyl)-); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 203, 448, 449, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —N(R⁴)— (e.g., —N(cyclopropyl)-); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 204, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-pyrazolyl); L is —N(R⁴)— (e.g., —N(cyclopropyl)-); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 205, 450, 451, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 5-methyloxazolyl); L is —N(R⁴)— (e.g., —N(CH₃)—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 206, 452, 453, or a pharmaceutically acceptable salt, solvate, hydrate, tautomer, or stereoisomer thereof.

In some embodiments, for Formula (I), A is bicyclic heterocyclyl (e.g., 1,6-diazaspiro[3.4]octanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 207, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methylthiazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 208, 454, 455, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methylpyridinyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 209, 456, 457, or a pharmaceutically acceptable salt, solvate, hydrate, tautomer, or stereoisomer thereof.

In some embodiments, for Formula (I), A is bicyclic heterocyclyl (e.g., 1,6-diazaspiro[3.5]nonanyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(Rh) (e.g., CH₂); n is 1; and m is 1. In some embodiments, the compound of Formulas (I), (I-a), (I-b), (I-c), (I-d), (I-e), (I-f), (I-g), and (I-i) is Compound 210, or a pharmaceutically acceptable salt, solvate, hydrate, tautomer, or stereoisomer thereof.

In some embodiments, for Formula (I), A is monocyclic heterocyclyl (e.g., 3-(N-cyclopropyl)aminopyrrolidinyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-d), (I-e), (I-f), (I-g), and (I-i) is Compound 211, 214, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(Rh) (e.g., CH₂); n is 1; and m is 1. In some embodiments, the compound of Formulas (I), (I-a), (I-b), (I-c), (I-d), (I-e), (I-f), (I-g), and (I-i) is Compound 212, 213, 257, 258, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 1H-imidazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 215, 458, 459, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., 2-methylpyridazin-3 (2H)-onyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 216, 460, 461, or a pharmaceutically acceptable salt, solvate, hydrate, tautomer, or stereoisomer thereof.

In some embodiments, for Formula (I), A is bicyclic heterocyclyl (e.g., 1,6-diazaspiro[3.4]octanyl); B is monocyclic heteroaryl (e.g., 1H-pyrazolyl); L is absent; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 217, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 1H-pyrazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 218, 462. 463, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 219, or a pharmaceutically acceptable salt, solvate, hydrate, tautomer, or stereoisomer thereof.

In some embodiments, for Formula (I), A is bicyclic heterocyclyl (e.g., 1-methyl-1,6-diazaspiro[3.4]octanyl); B is monocyclic heteroaryl (e.g., 1H-pyrazolyl); L is absent; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 220, or a pharmaceutically acceptable salt, solvate, hydrate, tautomer, or stereoisomer thereof.

In some embodiments, for Formula (I), A is bicyclic heterocyclyl (e.g., 1,7-diazaspiro[3.5]nonanyl); B is monocyclic heteroaryl (e.g., 1H-pyrazolyl); L is absent; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 221, or a pharmaceutically acceptable salt, solvate, hydrate, tautomer, or stereoisomer thereof.

In some embodiments, for Formula (I), A is bicyclic heterocyclyl (e.g., 6-methyl-1,6-diazaspiro[3.5]nonanyl); B is monocyclic heteroaryl (e.g., 1H-pyrazolyl); L is absent; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 222, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 3-methoxypyridazinyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 223, 464, 465, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 1H-pyrazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 224, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 3-methoxypyridazinyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 225, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 226, or a pharmaceutically acceptable salt, solvate, hydrate, tautomer, or stereoisomer thereof.

In some embodiments, for Formula (I), A is bicyclic heterocyclyl (e.g., 7-methyl-1,7-diazaspiro[3.5]nonanyl); B is monocyclic heteroaryl (e.g., 1H-pyrazolyl); L is absent; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 227, or a pharmaceutically acceptable salt, solvate, hydrate, tautomer, or stereoisomer thereof.

In some embodiments, for Formula (I), A is bicyclic heterocyclyl (e.g., (1R,5S)-1,5-dimethyl-8-azabicyclo[3.2.1]octanyl); B is monocyclic heteroaryl (e.g., 1H-pyrazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 228, 259, 466, 467, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is mon161yridine heterolyl (e.g., pyridin-2 (1H)-onyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 229, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 3-fluoro-1H-pyrazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 230, 468, 469, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —N(R⁴)— (e.g., —NCH₃—); Z¹, Z³, Z⁴, Z⁵ and Z⁶ are each independently C(R⁶) (e.g., CH); Z² is N; X is O; Y is C(R^{7a})(R^{7I}) (e.g., CH₂); n is 1; and m is 1. In some embodiments, the compound of Formulas (I), (I-a), (I-b), (I-c), (I-e), and (I-f) is Compound 231, or a pharmaceutically acceptable salt, solvate, hydrate, tautomer, or stereoisomer thereof.

In some embodiments, for Formula (I), A is bicyclic heterocyclyl (e.g., (1R,5S)-3-oxa-9-azabicyclo[3.3.1]nonanyl); B is monocyclic heteroaryl (e.g., 3-methoxypyridazinyl); L is —N(R⁴)— (e.g., —NCH₃—); Z¹, Z³, Z⁴, Z⁵ and Z⁶ are each independently C(R⁶) (e.g., CH); Z² is N; X is O; Y is C(R^{7a})(R^{7I}) (e.g., CH₂); n is 1; and m is 1. In some embodiments, the compound of Formulas (I), (I-a), (I-b), (I-c), (I-e), and (I-f) is Compound 232, 246, 470, 471, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —NR⁴— (e.g., —NCH₃—; Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 233, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 1H-pyrazolyl); L is —O—; Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 234, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 2H-1,2,3-triazolyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 235, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 1H-pyrazolyl); L is —O—; Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 236, 471, 472, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —N(R⁴)— (e.g., —NCH₃—); Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 237, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 2H-1,2,3-triazolyl); L is —NR⁴— (e.g., —NH—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 238, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 2H-1,2,3-triazolyl); L is —NR⁴— (e.g., —NCH₃—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 239, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 3-methoxypyridazinyl); L is —NR⁴— (e.g., —NH—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 240, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 1H-pyrazolyl); L is —NR⁴— (e.g., —NH—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 241, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 3-methoxypyridazinyl); L is —N(R⁴)— (e.g., —NCH₃—); Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 242, 245, 254, 473, 474, 477, 478, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 3-methoxypyridazinyl); L is —O—; Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 243, 475, 476, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —O—; Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 244, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 3-methoxypyridazinyl); L is —NR⁴— (e.g., —NCH₃—); Z¹, Z³, Z⁴, Z⁵ and Z⁶ are each independently C(R⁶) (e.g., CH); Z² is N; X is O; Y is C(R^{7a})(R^{7I}) (e.g., CH₂); n is 1; and m is 1. In some embodiments, the compound of Formulas (I), (I-a), (I-b), (I-c), (I-e), and (I-f) is Compound 247, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.g., 1H-pyrazolyl); L is —NR⁴— (e.g., —NCH₃—); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 248, or a pharmaceutically acceptable salt, solvate, hydrate, tautomer, or stereoisomer thereof.

In some embodiments, for Formula (I), A is monocyclic heterocyclyl (e.g., 3-(N-cyclopropyl)aminopyrrolidinyl); B is monocyclic heteroaryl (e.g., 2H-1,2,3-triazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 249, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 2H-1,2,3-triazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 250, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 251, 265, 269, and 323, or a pharmaceutically acceptable salt, solvate, hydrate, tautomer, or stereoisomer thereof.

In some embodiments, for Formula (I), A is monocyclic heterocyclyl (e.g., 3-(N-(1-methylcyclopropyl)aminopyrrolidinyl); B is monocyclic heteroaryl (e.g., 2H-1,2,3-triazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 252, or a pharmaceutically acceptable salt, solvate, hydrate, tautomer, or stereoisomer thereof.

In some embodiments, for Formula (I), A is monocyclic heterocyclyl (e.g., 3-(N-(1-methylcyclopropyl)aminopyrrolidinyl); B is monocyclic heteroaryl (e.g., 1H-pyrazolyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 253, or a pharmaceutically acceptable salt, solvate, hydrate, tautomer, or stereoisomer thereof.

In some embodiments, for Formula (I), A is monocyclic heterocyclyl (e.g., 3-(N-cyclopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 255, or a pharmaceutically acceptable salt, solvate, hydrate, tautomer, or stereoisomer thereof.

In some embodiments, for Formula (I), A is monocyclic heterocyclyl (e.g., 3-(N-(1-methylcyclopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 256, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 1H-pyrazolyl); L is —O—; Z¹, Z³, Z⁴, Z⁵ and Z⁶ are each independently C(R⁶) (e.g., CH of CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 260, 481, 482, or a pharmaceutically acceptable salt, solvate, hydrate, tautomer, or stereoisomer thereof.

In some embodiments, for Formula (I), A is bicyclic heterocyclyl (e.g., 6,6-difluoro-8-azabicyclo[3.2.1]octanyl); B is monocyclic heteroaryl (e.I2-methylpyridazin-3 (2H)-onyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), (I-f), and (I-i) is Compound 261, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-isopropoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 263, or a pharmaceutically acceptable salt, solvate, hydrate, tautomer, or stereoisomer thereof.

In some embodiments, for Formula (I), A is monocyclic heterocyclyl (e.g., 3-(N-cyclopropylmethyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 264, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-cyclopropylpyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 266, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-ethoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 267, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-cyclopropoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 268, or a pharmaceutically acceptable salt, solvate, hydrate, tautomer, or stereoisomer thereof.

In some embodiments, for Formula (I), A is monocyclic heterocyclyl (e.g., 3-(N-methyl-N-tert-butyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 270, 271, and 322, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., pyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 272, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-methylpyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 273, 274, and 314, or a pharmaceutically acceptable salt, solvate, hydrate, tautomer, or stereoisomer thereof.

In some embodiments, for Formula (I), A is monocyclic heterocyclyl (e.g., 3-(N-isopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 275, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-methoxypyridazinyl); L is —N(R⁴)— (e.g., —NCH₃—); Z³, Z⁴, Z⁵ and Z⁶ are each independently C(R⁶) (e.g., CH); Z¹ and Z² are N; X is O; Y is C(R^{7a})(R^{7b}) (e.g., CH₂); n is 1; and m is 1. In some embodiments, the compound of Formulas (I), (I-a), (I-b), and (I-f) is Compound 276, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., 3-hydroxypyridazinyl); L is —N(R⁴)— (e.g., —NCH₃—); Z³, Z⁴, Z⁵ and Z⁶ are each independently C(R⁶) (e.g., CH); Z¹ and Z² are N; X is O; Y is C(R^{7a})(R^{7b}) (e.g., CH₂); n is 1; and m is 1. In some embodiments, the compound of Formulas (I), (I-a), (I-b), and (I-f) is Compound 277, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-methoxypyridazinyl); Z³, Z⁴, Z⁵ and Z⁶ are each independently C(R⁶) (e.g., CH); Z¹ and Z² are N; X is O; Y is C(R^{7a})(R^{7b}) (e.g., CH₂); n is 1; and m is 1. In some embodiments, the compound of Formulas (I), (I-a), (I-b), and (I-f) is Compound 278, 279, or a pharmaceutically acceptable salt, solvate, hydrate, tautomer, or stereoisomer thereof.

In some embodiments, for Formula (I), A is monocyclic heterocyclyl (e.g., 3-(N-cyclobutyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 280, or a pharmaceutically acceptable salt, solvate, hydrate, tautomer, or stereoisomer thereof.

In some embodiments, for Formula (I), A is monocyclic heterocyclyl (e.g., 3-(N-cyclobutyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 280, or a pharmaceutically acceptable salt, solvate, hydrate, tautomer, or stereoisomer thereof.

In some embodiments, for Formula (I), A is monocyclic heterocyclyl (e.g., 3-(N-tert-pentyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 281, or a pharmaceutically acceptable salt, solvate, hydrate, tautomer, or stereoisomer thereof.

In some embodiments, for Formula (I), A is monocyclic heterocyclyl (e.g., piperazyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1.

In some embodiments, the compound of Formulas (I), (I-a), (I-b), (I-c), (I-e), and (I-f) is Compound 282, or a pharmaceutically acceptable salt, solvate, hydrate, tautomer, or stereoisomer thereof.

In some embodiments, for Formula (I), A is monocyclic heterocyclyl (e.g., octahydropyrrolo[1,2-a]pyrazinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 283, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-ethylpyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 284, or a pharmaceutically acceptable salt, solvate, hydrate, tautomer, or stereoisomer thereof.

In some embodiments, for Formula (I), A is monocyclic heterocyclyl (e.g., 3-(N-(1-methylcyclobutyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 285, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-isopropoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 286, or a pharmaceutically acceptable salt, solvate, hydrate, tautomer, or stereoisomer thereof.

In some embodiments, for Formula (I), A is monocyclic heterocyclyl (e.g., 2-methylpiperazyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(Rh) (e.g., CH₂); n is 1; and m is 1. In some embodiments, the compound of Formulas (I), (I-a), (I-b), (I-c), (I-e), and (I-f) is Compound 287, 324, and 325, or a pharmaceutically acceptable salt, solvate, hydrate, tautomer, or stereoisomer thereof.

In some embodiments, for Formula (I), A is monocyclic heterocyclyl (e.g., 4-aminopiperidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(Rh) (e.g., CH₂); n is 1; and m is 1. In some embodiments, the compound of Formulas (I), (I-a), (I-b), (I-c), (I-e), and (I-f) is Compound 288, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-methoxypyridazinyl); Z², Z³, Z⁴, Z⁵ and Z⁶ are each independently C(R⁶) (e.g., CH); Z¹ is N; X is O; Y is C(R^{7a})(R^{7b}) (e.g., CH₂); n is 1; and m is 1. In some embodiments, the compound of Formulas (I), (I-a), (I-b), and (I-f) is Compound 289, or a pharmaceutically acceptable salt, solvate, hydrate, tautomer, or stereoisomer thereof.

In some embodiments, for Formula (I), A is bicyclic heterocyclyl (e.g., (1R,5S)-3-oxa-9-azabicyclo[3.3.1]nonanyl); B is monocyclic heteroaryl (e.g., 3-methoxypyridazinyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 290, 483, 484, or a pharmaceutically acceptable salt, solvate, hydrate, tautomer, or stereoisomer thereof.

In some embodiments, for Formula (I), A is bicyclic heterocyclyl (e.g., (1R,5S)-9-methyl-3-oxa-9-azabicyclo[3.3.1]nonanyl); B is monocyclic heteroaryl (e.g., 3-methoxypyridazinyl); L is —O—; Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 291, 485, 486, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 4-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 292, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 4-cyclopropoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 293, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., NMe); Z³, Z⁴, and Z⁵ are each independently C(R⁶) (e.g., CH); Z¹ and Z² are N; X is O; Y is C(R^{7a})(R^{7b}) (e.g., CH₂); n and m are both 1; and R^{2a} and R^{2b} are each independently hydrogen. In some embodiments, the compound of Formulas (I), (I-a), (I-b), and (I-f) is Compound 294, 487, 488, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., 3-methoxypyridazinyl); L is —N(R⁴)— (e.g., NMe); Z³, Z⁴, and Z⁵ are each independently C(R⁶) (e.g., CH); Z¹ and Z² are N; X is O; Y is C(R^{7a})(R^{7I}) (e.g., CH₂); n and m are both 1; and R^{2a} and R^{2b} are each independently hydrogen.

In some embodiments, the compound of Formulas (I), (I-a), (I-b), and (I-f) is Compound 295, 489, 490, or a pharmaceutically acceptable salt, solvate, hydrate, tautomer, or stereoisomer thereof.

In some embodiments, for Formula (I), A is monocyclic heterocyclyl (e.g., 2-cyclopropylpiperazyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 296, 297, and 326, or a pharmaceutically acceptable salt, solvate, hydrate, tautomer, or stereoisomer thereof.

In some embodiments, for Formula (I), A is bicyclic heterocyclyl (e.g., (1R,5S)-8-azabicyclo[3.2.1]octanyl); B is monocyclic heteroaryl (e.g., 3-methoxypyridazinyl); L is —O—; Z³, Z⁴, Z⁵ and Z⁶ are each independently C(R⁶) (e.g., CH); Z¹ and Z² are N; X is O; Y is C(R^{7a})(R^{7b}) (e.g., CH₂); n is 1; and m is 1. In some embodiments, the compound of Formulas (I), (I-a), (I-b), and (I-f) is Compound 298, 491, 492, or a pharmaceutically acceptable salt, solvate, hydrate, tautomer, or stereoisomer thereof.

In some embodiments, for Formula (I), A is bicyclic heterocyclyl (e.g., (2,2,6,6-tetramethylpiperidinyl); B is monocyclic heteroaryl (e.g., 3-methoxypyridazinyl); L is —O—; Z³, Z⁴, Z⁵ and Z⁶ are each independently C(R⁶) (e.g., CH); Z¹ and Z² are N; X is O; Y is C(R^{7a})(R^{7b}) (e.g., CH₂); n is 1; and m is 1. In some embodiments, the compound of Formulas (I), (I-a), (I-b), and (I-f) is Compound 299, or a pharmaceutically acceptable salt, solvate, hydrate, tautomer, or stereoisomer thereof.

In some embodiments, for Formula (I), A is monocyclic heterocyclyl (e.g., 4-(N-methyl)aminopiperidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 300, or a pharmaceutically acceptable salt, solvate, hydrate, tautomer, or stereoisomer thereof.

In some embodiments, for Formula (I), A is monocyclic heterocyclyl (e.g., 4-(N-isopropyl)aminopiperidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 301, or a pharmaceutically acceptable salt, solvate, hydrate, tautomer, or stereoisomer thereof.

In some embodiments, for Formula (I), A is monocyclic heterocyclyl (e.g., 4-(N-tert-butyl)aminopiperidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 302, or a pharmaceutically acceptable salt, solvate, hydrate, tautomer, or stereoisomer thereof.

In some embodiments, for Formula (I), A is monocyclic heterocyclyl (e.g., 2-isopropylpiperazyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 303, 327, and 328, or a pharmaceutically acceptable salt, solvate, hydrate, tautomer, or stereoisomer thereof.

In some embodiments, for Formula (I), A is monocyclic heterocyclyl (e.g., 2-tert-butylpiperazyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 304, 317, and 318, or a pharmaceutically acceptable salt, solvate, hydrate, tautomer, or stereoisomer thereof.

In some embodiments, for Formula (I), A is monocylic heterocyclyl (e.g., 1,3′-bipyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 305, or a pharmaceutically acceptable salt, solvate, hydrate, tautomer, or stereoisomer thereof.

In some embodiments, for Formula (I), A is monocyclic heterocyclyl (e.g., 3-(N,N-dimethyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 306, or a pharmaceutically acceptable salt, solvate, hydrate, tautomer, or stereoisomer thereof.

In some embodiments, for Formula (I), A is monocyclic heterocyclyl (e.g., 3-(N-methyl-N-cyclobutyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 307, or a pharmaceutically acceptable salt, solvate, hydrate, tautomer, or stereoisomer thereof.

In some embodiments, for Formula (I), A is monocyclic heterocyclyl (e.g., 3-(N-methyl-N-methylcyclopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 308, or a pharmaceutically acceptable salt, solvate, hydrate, tautomer, or stereoisomer thereof.

In some embodiments, for Formula (I), A is monocyclic heterocyclyl (e.g., 3-methyl-3-(N-isopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 309, or a pharmaceutically acceptable salt, solvate, hydrate, tautomer, or stereoisomer thereof.

In some embodiments, for Formula (I), A is monocyclic heterocyclyl (e.g., 3-(N-methyl-N-ethyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 310, or a pharmaceutically acceptable salt, solvate, hydrate, tautomer, or stereoisomer thereof.

In some embodiments, for Formula (I), A is monocyclic heterocyclyl (e.g., 3-(N-methyl-N-isopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 311, or a pharmaceutically acceptable salt, solvate, hydrate, tautomer, or stereoisomer thereof.

In some embodiments, for Formula (I), A is monocyl75yridine175erocyclyl (e.g., 3-(azetidin-1-yl)pyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 312, or a pharmaceutically acceptable salt, solvate, hydrate, tautomer, or stereoisomer thereof.

In some embodiments, for Formula (I), A is bicyclic heterocyclyl (e.g., azabicyclo[3.2.1]octanyl); B is monocyclic heteroaryl (e.g., pyrazolyl); L is —N(R⁴)— (e.g., NH); Z¹, Z², Z³, Z⁴, Z⁵, and Z⁶ are each independently C(R⁶) (e.g., CH); X is C(R^{7a})(R^{7b}) (e.g., CH₂); Y is O; n and m are both 1; and R^{2a} and R^{2b} are each independently hydrogen. In some embodiments, the compound of Formulas (I), (I-a), (I-b), (I-c), (I-e), and (I-g) is Compound 313, 493, 494, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.I5-fluoropyridazin-3 (2H)-onyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 315, or a pharmaceutically acceptable salt, solvate, hydrate, tautomer, or stereoisomer thereof.

In some embodiments, for Formula (I), A is monocyclic heterocyclyl (e.g., 3-(N-(2,3-dimethylbutan-2-yl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 316, or a pharmaceutically acceptable salt, solvate, hydrate, tautomer, or stereoisomer thereof.

In some embodiments, for Formula (I), A is monocyclic heterocyclyl (e.g., 2-methyl-6-cyclopropylpiperazyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 319, 330, 331, or a pharmaceutically acceptable salt, solvate, hydrate, tautomer, or stereoisomer thereof.

In some embodiments, for Formula (I), A is monocyclic heterocyclyl (e.g., 3-(N-methyl-N-cyclopropyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 320, or a pharmaceutically acceptable salt, solvate, hydrate, tautomer, or stereoisomer thereof.

In some embodiments, for Formula (I), A is monocyclic heterocyclyl (e.g., 3-(N,N-diethyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 321, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-methoxypyridazinyl); Z², Z³, Z⁴ and Z⁶ are each independently C(R⁶) (e.g., CH); Z¹ and Z⁵ are N; X is O; Y is C(R^{7a})(R^{7b}) (e.g., CH₂); n is 1; and m is 1. In some embodiments, the compound of Formulas (I) and (I-a) is Compound 329, or a pharmaceutically acceptable salt, solvate, hydrate, tautomer, or stereoisomer thereof.

In some embodiments, for Formula (I), A is monocyclic heterocyclyl (e.g., 3-(N-tert-butyl)aminopyrrolidinyl); B is monocyclic heteroaryl (e.g., 3-methoxypyridazinyl); Z¹, Z³, Z⁴, Z⁵ and Z⁶ are each independently C(R⁶) (e.g., CH or CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 332, 335, 495, or a pharmaceutically acceptable salt, solvate, hydrate, tautomer, or stereoisomer thereof.

In some embodiments, for Formula (I), A is monocyclic heterocyclyl (e.g., 3-(N-methyl-N-tert-butyl)aminopyrrolidinyl); B is monocyclic heteroaryl (e.g., 3-methoxypyridazinyl); Z¹, Z³, Z⁵ and Z⁶ are each independently C(R⁶) (e.g., CH); Z⁴ is C(R⁶) (e.g., CF); Z² is N; X is O; Y is C(R^{7a})(R^{7b}) (e.g., CH₂); n is 1; and m is 1. In some embodiments, the compound of Formulas (I), (I-a), (I-b), (I-c), (I-e), and (I-f) is Compound 333, 334, and 496, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl or piperidinyl) or bicyclic heterocyclyl (e.g., azabicyclo[3.2.1]octanyl); B is monocyclic heteroaryl (e.g., pyrazolyl); L is —N(R⁴)— (e.g., NMe or NH) or O; Z¹, Z³, Z⁴, Z⁵, and Z⁶ are each independently C(R⁶) (e.g., CH); Z² is N or C(R⁶); one of X and Y is C(R^{7a})(R^{7b}) (e.g., CH₂), and the other of X and Y is O; n is 1 or 2; m is 1 or 2; and R^{2a} and R^{2b} are each independently hydrogen. In some embodiments, the compound of Formulas (I), (I-a), (I-d), and (I-e) and is Compound 116, or a pharmaceutically acceptable salt, solvate, hydrate, tautomer, or stereoisomer thereof. In some embodiments, the compound of Formulas (I), (I-b), (I-c), and (I-e) and is one of Compounds 117-119, 121-125, or 127-132, or a pharmaceutically acceptable salt, solvate, hydrate, tautomer, or stereoisomer thereof.

In some embodiments, any one of Formulas (I), (I-a), (I-b), (I-c), (I-d), (I-e), (I-f), (I-g), (I-h), and (I-i), A is monocyclic heterocyclyl (e.g., 2,2,6,6-tetramethylpiperidinyl or piperidinyl) or bicyclic heterocyclyl (e.g., azabicyclo[3.2.1]octanyl); B is monocyclic heteroaryl (e.g., pyrazolyl, imidazolyl, or methylimidazolyl) or monocyclic heterocyclyl; L is L is —N(R⁴)— (e.g., N(CH₃) or N(CD₃)); X is O; Y is C(R^{7a})(R^{7b}) (e.g., CH₂); and Z¹, Z³, Z⁴, Z⁵, and Z⁶ are each independently C(R⁶) (e.g., CH); Z² is N. In some embodiments, the compound of any one of Formulas (I), (I-a), (I-b), (I-c), (I-d), (I-e), (I-f), (I-g), (I-h), and (I-i) is a compound selected from 108, 116, 121, 143, 149, 150, 151, 153, 155, 158, 159, 160, 162, 163, 164, 169, 178, 180, 190, 200, and 201, or a pharmaceutically acceptable salt, solvate, hydrate, tautomer, or stereoisomer thereof. In some embodiments, the compound of any one of Formulas (I), (I-a), (I-b), (I-c), (I-d), (I-e), (I-f), (I-g), (I-h), and (I-i) is a compound selected from Compound 108, 150, 151, 153, 155, 159, 160, 162, 163, 164, 178, 180, 190, and 200, or a pharmaceutically acceptable salt, solvate, hydrate, tautomer, or stereoisomer thereof. In some embodiments, the compound is Compound 108 or a pharmaceutically acceptable salt, solvate, hydrate, tautomer, or stereoisomer thereof. In some embodiments, the compound is Compound 150 or a pharmaceutically acceptable salt, solvate, hydrate, tautomer, or stereoisomer thereof. In some embodiments, the compound is Compound 151 or a pharmaceutically acceptable salt, solvate, hydrate, tautomer, or stereoisomer thereof. In some embodiments, the compound is Compound 153 or a pharmaceutically acceptable salt, solvate, hydrate, tautomer, or stereoisomer thereof. In some embodiments, the compound is Compound 155 or a pharmaceutically acceptable salt, solvate, hydrate, tautomer, or stereoisomer thereof. In some embodiments, the compound is Compound 159 or a pharmaceutically acceptable salt, solvate, hydrate, tautomer, or stereoisomer thereof. In some embodiments, the compound is Compound 160 or a pharmaceutically acceptable salt, solvate, hydrate, tautomer, or stereoisomer thereof. In some embodiments, the compound is Compound 162 or a pharmaceutically acceptable salt, solvate, hydrate, tautomer, or stereoisomer thereof. In some embodiments, the compound is Compound 163 or a pharmaceutically acceptable salt, solvate, hydrate, tautomer, or stereoisomer thereof. In some embodiments, the compound is Compound 164 or a pharmaceutically acceptable salt, solvate, hydrate, tautomer, or stereoisomer thereof. In some embodiments, the compound is Compound 178 or a pharmaceutically acceptable salt, solvate, hydrate, tautomer, or stereoisomer thereof. In some embodiments, the compound is Compound 180 or a pharmaceutically acceptable salt, solvate, hydrate, tautomer, or stereoisomer thereof. In some embodiments, the compound is Compound 190 or a pharmaceutically acceptable salt, solvate, hydrate, tautomer, or stereoisomer thereof. In some embodiments, the compound is Compound 200 or a pharmaceutically acceptable salt, solvate, hydrate, tautomer, or stereoisomer thereof.

### Pharmaceutical Compositions, Kits, and Administration

The present invention provides pharmaceutical compositions comprising a compound of Formula (I) e.g., a compound of Formula (I) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or a pharmaceutically acceptable salt thereof, and optionally a pharmaceutically acceptable excipient. In certain embodiments, the compound of Formula (I) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension.

These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I) may be used to alter the amount, structure, or composition of a nucleic acid (e.g., a PREcursor RNA, e.g., a PRE-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a PREcursor RNA, e.g., a pre-mRNA) through inclusion of a splice site in the target, wherein the method comprises providing a compound of Formula (I). In some embodiments, inclusion of a splice site in a target (e.g., a PREcursor RNA, e.g., a PRE-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a PREcursor RNA, e.g., a PRE-mRNA, or the resulting mRNA) through exclusion of a splice site in the target, wherein the method comprises providing a compound of Formula (I). In some embodiments, exclusion of a splice site in a target (e.g., a PREcursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a PREcursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6VIG3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf101, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, CIR, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENP1, CENP1, CENTB2, CENTG2, CEP110, CEP170, CEP192, CETP, CFB, CFTR, CFH, CGN, CGNL1, CHAF1A, CHD9, CHIC2, CHL1, CHN1, CHM, CLEC 16A, CLIC2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17A1, COL19A1, COL1A1, COL1A2, COL2A1, COL3A1, COL4A1, COL4A2, COL4A5, COL4A6, COL5A2, COL6A1, COL7A1, COL9A1, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, cTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUNID4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMT1, DNTTIP2, DOCK4, DOCK5, DOCK10, DOCKI1, DOT1L, DPP3, DPP4, DPY19L2P2, DR1, DSCC1, DVL3, DUX4, DYNCIH1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBXO18, FBXO38, FCGBP, FECH, FEZ2, FGA, FGD6, FGFR2, FGFRIOP, FGFRIOP2, FGFR2, FGG, FGR, FIX, FKBP3, FLI1, FLJ35848, FLJ36070, FLNA, FN1, FNBP1L, FOLH1, FOSL1, FOSL2, FOXK1, FOXM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNAI1, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IF144L, IKBKAP, IKZF1, IKZF3, ILIR2, ILSRA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, ITGB3, ITGB4, ITIH1, ITPR2, IWS1, JAK1, JAK2, JAG1, JMJDIC, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMEN1, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, IMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF212, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MP1, MRAP2, MRPL11, MRPL39, MRPS28, MRPS35, MS4A13, MSH2, MSH3, MSMB, MSTIR, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATO2, NFE212, NF1A, NF1B, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NRID1, NRIH3, NRIH4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPLI1, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PDIA3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGE, PIGN, PIGT, PIK302G, PIK3CA, PIK3CD, PIK3CG, PIK3R1, PIP5K1A, PITRM1, PIWIL3, PKD1, PKHDIL1, PKD2, PK1B, PKLR, PKM1, PKM2, PLAGL2, PLCB1, PLCB4, PLCG1, PLD1, PLEKHA5, PLEKHA7, PLEKHM1, PLKR, PLXNC1, PMEBP1, POLN, POLR3D, POMT2, POSTN, POU2AF1, POU2F2, POU2F3, PPARA, PPF1A2, PPPIR12A, PPP3CB, PPP4C, PPP4R1L, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPNI1, PTPN22, PTPRD, PTPRK, PTPRM, PTPRN2, PTPRT, PUS10, PVRL2, PYGM, QRSL1, RAB11FIP2, RAB23, RAF1, RALBP1, RALGDS, RBICC1, RBL2, RBM39, RBM45, RBPJ, RBSN, REC8, RELB, RFC4, RFT1, RFTN1, RHOA, RHPN2, RIF1, RIT1, RLN3, RMND5B, RNF11, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRPIB, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SELIL, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPAIL2, SIPAIL3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SP110, SPAG9, SPATA13, SPATA4, SPATS1, SPECC1L, SPDEF, SPI1, SPINK5, SPP2, SPTA1, SRF, SRM, SRP72, SSX3, SSX5, SSX9, STAG1, STAG2, STAMBPL1, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBCID3G, TBCID8B, TBCID26, TBCID29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOMIL1, TOMIL2, TOP2B, TP53, TP53INP1, TP53BP2, TP5313, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGTIA1, UHRFIBP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VTI1A, VT11B, VWA3B, WDFY2, WDR16, WDR17, WDR26, WDR44, WDR67, WDTC1, WRN, WRN1P1, WT1, WWC3, XBP1, XRN1, XRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include AICF, A4GALT, AAR2, ABAT, ABCA11P, ZNF721, ABCA5, ABHD10, ABHD13, ABHD2, ABHD6, AC000120.3, KRIT1, AC004076.1, ZNF772, AC004076.9, ZNF772, AC004223.3, RAD51D, AC004381.6, AC006486.1, ERF, AC007390.5, AC007780.1, PRKAR1A, AC007998.2, INO80C, AC009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDDIA, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHDIL, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKRIA1, AKT1, AKTIS1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDHIB1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127120.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5 MG, AP002495.2, AP003175.1, OR2AT4, AP003419.1, CLCF1, AP005263.1, ANKRD12, AP006621.5, AP006621.1, APIG1, AP3 M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP 19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATPIB3, ATP2C1, ATP5F1A, ATP5G2, ATP5J, ATP5MD, ATP5 PF, ATP6AP2, ATP6V0B, ATP6V1C1, ATP6V1D, ATP7B, ATXN1, ATXN1L, IST1, ATXN3, ATXN7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOCIS1, RP11-644F5.10, BLOC IS6, AC090527.2, BLOCIS6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUBIB-PAK6, PAK6, BUB3, C10orf68, C11orfl, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, (19orf12, C19orf40, C19orf47, C19orf48, C19orf54, CID, CIGALT1, CIQB, CIQTNF1, CIS, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN1, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB11P1, CCNC, CCND3, CONG1, CCP110, CCR9, CCT7, CCT8, CD151, CDID, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CH₁A, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RPI-309K20.6, RBM12, CPNE3, CPSF3L, CPTIC, CREB3L2, CREM, CRP, CRYZ, CS, AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP 1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLREIC, DCTD, DCTN1, DCTN4, DCUNID2, DDR1, DDXI1, DDX19B, AC012184.2, DDX19B, RP11-529K1.3, DDX25, DDX39B, ATP6VIG2-DDX39B, SNORD84, DDX42, DDX60L, DEDD, DEDD2, DEFA1, DEFAIB, DEFAIB, DEFA3, DENNDIC, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASEIL1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECS1T, ECT2, EDC3, EDEM1, EDEM2, MMP24-AS1, RP4-61404.11, EEFIAKNMT, EEFID, EFEMP1, EFHC1, EGFL7, EHF, E124, EIFIAD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB4IL1, EPDR1, NME8, EPHX1, EPM2A, EPN1, EPN2, EPN3, EPS8L2, ERBB3, ERC1, ERCC1, ERG, ERI2, ERI2, DCUNID3, ERLIN2, ERMARD, ERRFI1, ESR2, RP11-544120.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOXM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLB1L, GLI1, GLT8D1, GMFG, GMPR2, GNA12, GNAQ, GNB1, GNB2, GNE, GNG2, GNGT2, GNPDA1, GNPDA2, GOLGA3, CHER, GOLGA4, GOLPH3L, GOLT1B, GPBPIL1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCYIA3, GUCYIB3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFCIR1, HCK, HDAC2, HDAC6, HDAC7, HDLBP, HEATR4, HECTD4, HEXIM2, HHAT, HHATL, CCDC13, HINFP, HIRA, C22orf39, HIVEP 3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IF127, IF144, IFT20, IFT22, IFT88, IGF2, INS-IGF2, IGF2BP3, IGFBP6, IKBKAP, IKBKB, IL11, IL18BP, IL18RAP, ILIRAP, ILIRL1, IL18R1, ILIRN, IL32, IL411, NUP62, AC011452.1, IL411, NUP62, CTC-326K19.6, IL6ST, ILVBL, IMMP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGBIBP1, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RP11-849F2.7, KRIT1, KRT15, KRT8, KIN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, IMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRR (39, LRRC42, LRRC48, LRRC4C, LRRC8A, LRRC8B, LRRD1, LRTOMT, LRTOMT, AP000812.5, LSM7, LTB4R, LTBP 3, LUC7L2, FMCI-LUC7L2, LUC7L3, LUZP1, LYG1, LYL1, LYPD4, LYPD6B, LYRM1, LYRM5, LYSMD4, MACC1, MADIL1, MADIL1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MEN2, MFSD2A, MGST3, MIB2, MICAL1, MICAL3, MICOS10, NBL1, MICOS10-NBL1, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAPIL1, NAPIL4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NRIH3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORA12, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARP11, PARVG, PASK, PAX6, PBRM1, PBXIP1, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIK1L, PDLIM7, PDP 1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PK1A, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POCIB, POFUT1, POLB, POLD1, POLH, POL1, POLL, POLR1B, POMI21, POM121C, AC006014.7, POM121C, AC211429.1, POMC, POMT1, POP1, PORCN, POU5F1, PSORSIC3, PPARD, PPARG, PPHLN1, PPIL3, PPIL4, PPM1A, PPM1B, AC013717.1, PPP1CB, PPP1R11, PPP1R13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L.3-RFFL, RAD51D, RAD52, RAE1, RAI14, RAI2, RALBP1, RAN, RANGAP1, RAP1A, RAP1B, RAPIGAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMYIE, RBPJ, RBPMS, RBSN, RCBTB2, RCC1, ROC1, SNHG3, RCCD1, RECOL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-(17orf49, RNF111, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8, CDK11A, RP11-120M18.2, PRKAR1A, RP11-133K1.2, PAK6, RP11-164J13.1, CAPN3, RP11-21J18.1, ANKRD12, RP11-322E11.6, INO80C, RP11-337 (18.10, CHD1L, RP11-432B6.3, TRIM59, RP11-468E2.4, IRF9, RP11-484M3.5, UPK1B, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-1021120.4, ZNF410, RP6-109B7.3, FLJ27365, RPE, RPH3AL, RPL15, RPL17, RPL17-(18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSLID1, RSRC2, RSRP 1, RUBCNL, RUNXIT1, RUVBL2, RWDD1, RWDD4, S100A13, AL162258.1, S100A13, RPI-178F15.5, S100A16, S100A4, S100A3, S100A6, S100PBP, SAA1, SACM1L, SAMD4B, SAR1A, SARAF, SARNP, RP11-76217.5, SCAMP5, SCAP, SCAPER, SCFD1, SCGB3A2, SCIN, SCML1, SONNID, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLENL1, SLMO1, SLTM, SLU7, SMAD2, SMAP 2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECCIL-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX21P, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, ST1L, STK25, STK33, STK38L, STK40, STMN1, STON1, STONI-GTF2A1L, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULTIA1, SULTIA2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAFIC, TAF6, AC073842.2, TAF6, RP11-506M12.1, TAF9, TAGLN, TANK, TAPSAR1, PSMB9, TAPT1, TATDN1, TAZ, TBCID1, TBCID12, HELLS, TBCID15, TBCID3H, TBCID3G, TBC1D5, TBCID5, SATB1, TBCA, TBCEL, TBCEL, AP000646.1, TBLIXR1, TBP, TBX5, TBXAS1, TCAF1, TCEA2, TCEAL4, TCEAL8, TCEAL9, TCEANC, TCEB1, TCF19, TCF25, TCF4, TCP1, TCPIOL, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF19, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOMIL1, TOPIMT, TOP3B, TOX2, TP53, RP11-199F11.2, TP53I11, TP531NP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE21, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWASA, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, XRCC3, XRN2, XXYLT1, YIF1A, YIF1B, YIPF1, YIPF5, YPEL5, YWHAB, YWHAZ, YYIAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158113.2, ZNF512B, ZNF512B, SAMD10, ZNF521, ZNF532, ZNF544, AC020915.5, ZNF544, CTD)-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOXM1 gene.

Exemplary genes that may be modulated by the compounds of Formula (I) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an mA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CcAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CuGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GaGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GuAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UaCguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UcCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UgGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GaGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugaggc (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacace (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugegge (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccugc (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugaggc (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucuege (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugeg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugagge (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUguccgcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugcguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory proteiN (kSRP), L hnRNP, LUC7L, M hnRNP, mBBP, muscle-blind like (mbnl), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SFl/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, Ul snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AIL) (e.g., B-cell AIL, T-cell AIL), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis.

Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) increases expression of the haploinsufficient gene locus. In an embodiment, a compound of Formula (I) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or a pharmaceutically acceptable salt thereof, e.g., as described herein, has a cell permeability measurement (Papp) of <2×10⁻⁶ cm s⁻¹. In an embodiment, a compound of Formula (I) or a pharmaceutically acceptable salt thereof, e.g., as described herein, has a cell permeability measurement (Papp) of between 2-6×10⁻⁶ cm s⁻¹. In an embodiment, a compound of Formula (I) or a pharmaceutically acceptable salt thereof, e.g., as described herein, has a cell permeability measurement (Papp) of Papp greater than 6×10⁻⁶ cm s⁻¹. In an embodiment, a compound of Formula (I) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or a pharmaceutically acceptable salt thereof, e.g., as described herein, has a cell efflux ratio of less than 1.5. In an embodiment, a compound of Formula (I) or a pharmaceutically acceptable salt thereof, e.g., as described herein, has a cell efflux ratio of between 1.5 and 5. In an embodiment, a compound of Formula (I) or a pharmaceutically acceptable salt thereof, e.g., as described herein, has a cell efflux ratio greater than 5. In an embodiment, a compound of Formula (I) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, modulates the expression of a target protein (e.g., HTT or MYB) in a reference cell or sample. In an embodiment, a compound of Formula (I) or a pharmaceutically acceptable salt thereof, e.g., as described herein, increases the expression of a target protein (e.g., HTT or MYB) in a reference cell or sample. In an embodiment, a compound of Formula (I) or a pharmaceutically acceptable salt thereof, e.g., as described herein, decreases the expression of a target protein (e.g., HTT or MYB) in a reference cell or sample. The effect of an exemplary compound of Formula (I) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I) or a pharmaceutically acceptable salt thereof, e.g., as described herein, has a protein abundance response less than 100 nM. In an embodiment, a compound of Formula (I) or a pharmaceutically acceptable salt thereof, e.g., as described herein, has a protein abundance response between 100-1000 nM. In an embodiment, a compound of Formula (I) or a pharmaceutically acceptable salt thereof, e.g., as described herein, has a protein abundance response greater than 1000 nM. In an embodiment, a compound of Formula (I) or a pharmaceutically acceptable salt thereof, e.g., as described herein, has a protein abundance response greater than 10 uM. In an embodiment, a compound of Formula (I) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, modulates the viability of a target cell in a subject or sample. In an embodiment, a compound of Formula (I) or a pharmaceutically acceptable salt thereof, e.g., as described herein, increases the viability of a target cell in a subject or sample. In an embodiment, a compound of Formula (I) or a pharmaceutically acceptable salt thereof, e.g., as described herein, decreases the viability of a target cell in a subject or sample. In an embodiment, a compound of Formula (I) or a pharmaceutically acceptable salt thereof, e.g., as described herein, does not impact the viability of a cell (e.g., is non-toxic) in a subject or sample. The effect an exemplary compound of Formula (I) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I) or a pharmaceutically acceptable salt thereof, e.g., as described herein, is tolerated by a target cell at a concentration of less than 100 nM. In an embodiment, a compound of Formula (I) or a pharmaceutically acceptable salt thereof, e.g., as described herein, is tolerated by a target cell at a concentration of between 100-1000 nM. In an embodiment, a compound of Formula (I) or a pharmaceutically acceptable salt thereof, e.g., as described herein, is tolerated by a target cell at a concentration of greater than 1000 nM. In an embodiment, a compound of Formula (I) or a pharmaceutically acceptable salt thereof, e.g., as described herein, is tolerated by a target cell at a concentration of greater than 10 uM.

In some embodiments, a compound of Formula (I) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I) or a pharmaceutically acceptable salt thereof, e.g., as described herein, has a Kp value of greater than 5. In an embodiment, a compound of Formula (I) or a pharmaceutically acceptable salt thereof, e.g., as described herein, has a Kp value between 1 and 5. In an embodiment, a compound of Formula (I) or a pharmaceutically acceptable salt thereof, e.g., as described herein, has a Kp value between 0.2-1. In an embodiment, a compound of Formula (I) or a pharmaceutically acceptable salt thereof, e.g., as described herein, has a Kp value of less than 0.2. In an embodiment, a compound of Formula (I) or a pharmaceutically acceptable salt thereof, e.g., as described herein, has a Kpuu value of greater than 2.5. In an embodiment, a compound of Formula (I) or a pharmaceutically acceptable salt thereof, e.g., as described herein, has a Kpuu value between 0.5-2.5. In an embodiment, a compound of Formula (I) or a pharmaceutically acceptable salt thereof, e.g., as described herein, has a Kpuu value between 0.1-0.5. In an embodiment, a compound of Formula (I) or a pharmaceutically acceptable salt thereof, e.g., as described herein, has a Kpuu value of less than 0.1. In an embodiment, a compound of Formula (I) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I) or a pharmaceutically acceptable salt thereof, e.g., as described herein, exhibits selectivity for HTT, e.g., an HTT-related nucleic acid sequence. In an embodiment, a compound of Formula (I) or a pharmaceutically acceptable salt thereof, e.g., as described herein, exhibits selectivity for SMN2, e.g., an SMN2-related nucleic acid sequence. In an embodiment, a compound of Formula (I) or a pharmaceutically acceptable salt thereof, e.g., as described herein, exhibits selectivity for Target C, e.g., a Target C-related nucleic acid sequence. In an embodiment, a compound of Formula (I) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I) or a pharmaceutically acceptable salt thereof, e.g., as described herein, has a 3-fold greater selectivity for HTT over MYB. In an embodiment, a compound of Formula (I) or a pharmaceutically acceptable salt thereof, e.g., as described herein, has a 3-fold greater selectivity for MYB over HTT. In an embodiment, a compound of Formula (I) or a pharmaceutically acceptable salt thereof, e.g., as described herein, has a 10-fold greater selectivity for HTT over MYB. In an embodiment, a compound of Formula (I) or a pharmaceutically acceptable salt thereof, e.g., as described herein, has a 10-fold greater selectivity for MYB over HTT. In an embodiment, a compound of Formula (I) or a pharmaceutically acceptable salt thereof, e.g., as described herein, has a 3-fold greater selectivity for HTT over SMN2. In an embodiment, a compound of Formula (I) or a pharmaceutically acceptable salt thereof, e.g., as described herein, has a 3-fold greater selectivity for SMN2 over HTT. In an embodiment, a compound of Formula (I) or a pharmaceutically acceptable salt thereof, e.g., as described herein, has a 10-fold greater selectivity for HTT over SMN2. In an embodiment, a compound of Formula (I) or a pharmaceutically acceptable salt thereof, e.g., as described herein, has a 10-fold greater selectivity for SMN2 over HTT. In an embodiment, a compound of Formula (I) or a pharmaceutically acceptable salt thereof, e.g., as described herein, has a 3-fold greater selectivity for MYB over SMN2. In an embodiment, a compound of Formula (I) or a pharmaceutically acceptable salt thereof, e.g., as described herein, has a 3-fold greater selectivity for SMN2 over MYB. In an embodiment, a compound of Formula (I) or a pharmaceutically acceptable salt thereof, e.g., as described herein, has a 10-fold greater selectivity for MYB over SMN2. In an embodiment, a compound of Formula (I) or a pharmaceutically acceptable salt thereof, e.g., as described herein, has a 10-fold greater selectivity for SMN2 over MYB. In an embodiment, a compound of Formula (I) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject.

Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (6) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C18 (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN).

Analytical chiral HPLC: Analytical chiral HPLC was performed on a Agilent 1260 using column: CHIRALPAK IG-3, CHIRALPAK IC-3 or CHIRALPAK OJ-3, with flow rate=1.2 mL/min. Mobile phase=MTBE(DEA):EtOH=50:50).

Preparative HPLC or Reverse Phase Flash Chromatography purification: prep-HPLC or reverse phase flash purification was performed using one of the following conditions:

Condition 1: Column: Xselect CSH OBD Column 30 mm×150 mm, Sum, n; Mobile Phase A: water (10 mmol/L NH₄HCO₃), Mobile Phase B: acetonitrile; Flow rate: 60 mL/min; Gradient 1: 5 B to 55 B in 8 min; Gradient 2: 30 B to 60 B in 8 min; Gradient 3: 3 B to 33 B in 8 min; Gradient 4: 15% B to 40% B in 8 min; Gradient 5: 3% B to 73% B in 8 min; Gradient 6: 25% B to 58% B in 8 min.

Condition 2: Column: XBridge Prep OBD C18 Column, 30×150 mm, Sum; Mobile Phase A: Water (10 mmol/L NH₄HCO₃), Mobile Phase B: acetonitrile; Flow rate: 60 mL/min; Gradient 1:10% B to 50% B in 8 min; Gradient 2: 5% B to 50% B in 6 min; Gradient 3: 5% B to 35% B in 6 min; Gradient 4: 10% B to 35% B in 8 min; Gradient 5: 25% B to 57% B in 8 min; Gradient 6: 15% B to 55% B in 8 min; Gradient 7: 5% B to 45% B in 8 min; Gradient 8: 5% B to 40% B in 8 min; Gradient 9: 5% B to 35% B in 8 min; Gradient 10: 50% to 80% B in 7 min; Gradient 11: 30% to 60% B in 7 min; Gradient 12: 12% to 20% B in 7 min. Gradient 13: 5% B to 55% B in 8 min; Gradient 14: 10% B to 90% B in 8 min.

Condition 3: Column: Xselect CSH OBD Column 30×150 mm Sum, n; Mobile Phase A: water (0.05% HCl); Mobile Phase B: acetonitrile; Flow rate: 60 mL/min; Gradient 1: 5 B to 35 B in 6 min; Gradient 2: 10 B to 29 B in 6 min. Gradient 3: 55% B to 40% B in 8 min.

Condition 4: Column YMC-Actus Triart C18, 30*150 mm, 5 μm; Mobile Phase A: water; Mobile Phase B: 10 mmol/L NH₄HCO₃ in acetonitrile; Gradient: 10% B up to 70% B in 8 min.

Condition 5: Column: XBridge C18 Silica gel, 19×150 mm; Mobile Phase A: water 0.05% NH₃H₂O; Mobile Phase B: acetonitrile. Gradient 1: 55% B to 85% B in 7 min. Gradient 2: 50% B to 80% B in 7 min. Gradient 3: 44% B to 71% B in 8 min. Gradient 4: 25% B to 55% B in 7 min. Gradient 5: 10% B to 50% B in 12 min,

Condition 6: Column: SunFire Prep C18 OBD Column 19×150 mm, 5 m 10 nm; Mobile phase A: water (0.05% TFA); Mobile Phase B: acetonitrile; Gradient 1: 19% B to 42% B in 7 min. Gradient 2: 15% B to 25% B in 10 min. Gradient 3: 10% B to 20% B in 7 min. Gradient 4: 22% B to 42% B in 9 min. Gradient 5: 10% B to 25% b in 10 min.

Condition 7: Column: XBridge Prep OBD Column 19×150 mm 8 μm; Mobile Phase A: water (0.05% NH₃·H₂O); Mobile Phase B: acetonitrile; Flow rate: 20 mL/min; Gradient 1: 27% B to 52% B in 8 min. Gradient 2: 15% B to 56% B in 8 min. Gradient 3: 22% B to 56% B in 8 min. Gradient 4: 20% B to 50% B in 8 min.

Condition 8: Column: Weich Ultimate XB-C18 50×250 mm 10 um; Mobile Phase A: water (0.1% NH₃·H₂O); Mobile Phase B: acetonitrile; Flow rate: 90 mL/min; Gradient 1: 15% B to 51% B in 12 min.

Condition 9: Column: Weich Ultimate XB-C18 50×250 mm 10 um; Mobile Phase A: water (0.1% TFA); Mobile Phase B: acetonitrile; Flow rate: 90 mL/min; Gradient 1: 15% B to 51% B in 12 min.

Condition 10: Column YMC-Actus Triart C18, 20*150 mm, 5 μm; Mobile Phase A: water (10 mmol/L NH₄HCO₃); Mobile Phase B: acetonitrile; Gradient 1: 20% B up to 65% B in 8 min; Gradient 2: 35% B to 65% B in 8 min; Gradient 3: 25% B to 63% B in 8 min, Gradient 4: 10% B to 60% B in 8 min; Gradient 5: 20% B to 70% B in 8 min; Condition 6: 25% B to 68% B in 10 min.

Condition 11: Column, XBridge Shield RP18 OBD Column, 19*150 mm, 5 μm; Mobile Phase A: water (10 mmol/L NH₄HCO₃); Mobile Phase B: acetonitrile; Gradient 1: 15% B to 55% B in 10 min; Gradient 2: 20% B to 65% B in 8 min; Gradient 3: 10% B to 50% B in 10 min; Gradient 4: 15% B to 60% B in 8 min; Gradient 5: 5% B to 45% B in 8 min; Gradient 6: 10% B to 70% B in 10 min; Gradient 7: 20% B to 55% B in 8 min; Gradient 8: 5% B to 60% B in 10 min; Gradient 9: 15% B to 65% B in 8 min; Gradient 10: 20% B to 75% B in 8 min.

Condition 12: Column, XBridge BEH C18 OBD Prep Column, 19*250 mm, 5 μm; Mobile Phase A: water (10 mmol/L NH₄HCO₃); Mobile Phase B: acetonitrile; Gradient 1: 15% B to 51% B in 8 min; Gradient 2: 15% B to 42% B in 10 min.

Condition 13: Column, XSelect CSH Prep C18 OBD, 19*150 mm, 5 μm; Mobile Phase A: water (10 mmol/L NH₄HCO₃), Mobile Phase B: acetonitrile; Gradient 1: 15% B to 70% B in 8 min.

Condition 14: Column, XSelect CSH Prep C18 OBD, 19*150 mm, 5 μm; Mobile Phase A: water (0.05% HCl), Mobile Phase B: acetonitrile; Flow rate: 60 mL/min; Gradient 1: 10% B to 35% B in 8 min; Gradient 2: 5% B to 32% B in 8 min.

Condition 15: Column, C18; Mobile Phase A: water (0.1% TFA); Mobile Phase B: acetonitrile; Gradient 1: 10% B to 50% B in 10 min.

Condition 16: Column: Kinetex EVO C18 Column, 30*150, 5 um; Mobile Phase A: water (10 mmol/L NH₄HCO₃), Mobile Phase B: acetonitrile; Flow rate: 60 mL/min; Gradient 1: 15% B to 44% B in 8 min; Gradient 2: 10% B to 40% B in 8 min.

Preparative chiral HPLC: purification by chiral HPLC was performed on a Gilson-GX 281 using column: CHIRALPAK IG-3, CHIRALPAK IC-3 or CHIRALPAK OJ-3.

Condition 1: Column: CHIRALPAK IG, 2*25 cm, 5 m; Mobile Phase A: MtBE (0.1% DEA), Mobile Phase B: methanol; Flow rate: 20 mL/min; Gradient 1: 50% B to 50% B in 19 min.

Condition 2: Column, YMC-Actus Triart C18, 30*150 mm, 5 um; Mobile Phase A: water (10 mmol/L NH₄HCO₃); Mobile Phase B: acetonitrile; Gradient 1: 20% B to 50% B in 10 min.

Condition 3: Column: CHIRAL ART Cellulose-SB, 4.6*100 mm, 3.0 um; Mobile Phase A: MtBE (0.1% DEA); Mobile Phase B: methanol Flow rate: 1 mL/min; Gradient: 30% B to 30% B.

### General Synthetic Scheme

Compounds of the present disclosure may be prepared using a synthetic protocol illustrated in Schemes A-D below.

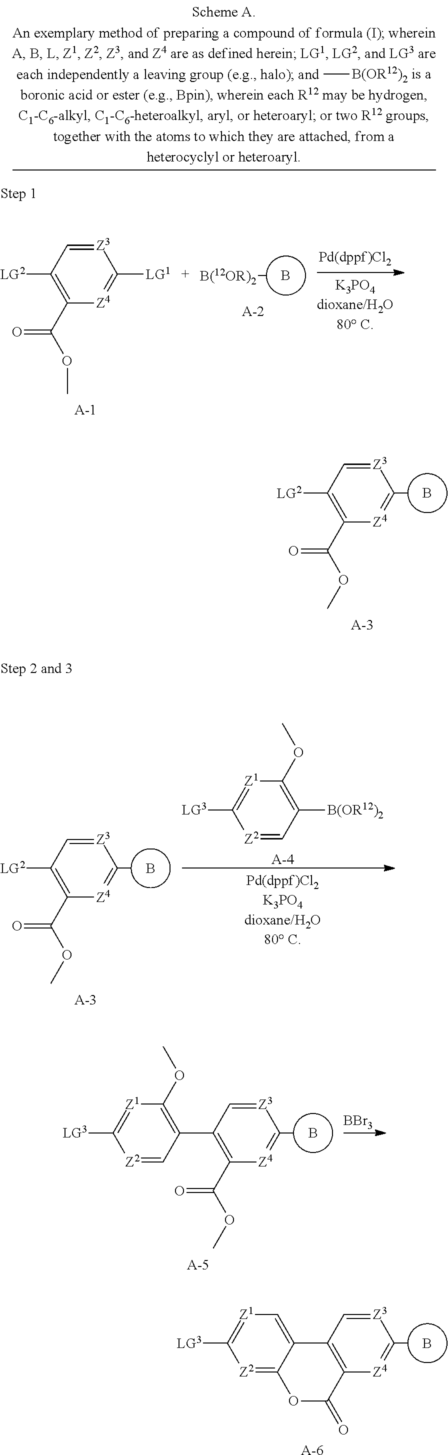

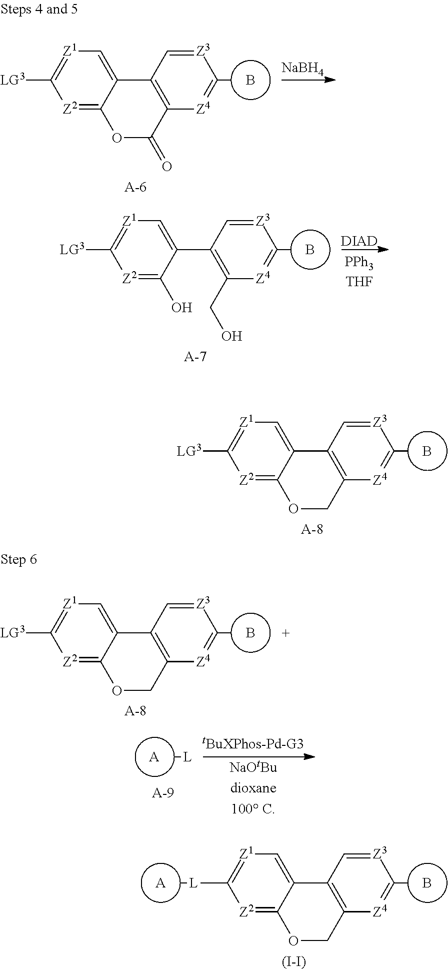

An exemplary method of preparing a compound described herein, such as a compound of Formula (I-I), is provided in Scheme A. In this scheme, A-3 is prepared in Step 1 by incubating A-1 with A-2 in the presence of 1,1′-bis(diphenylphosphino)ferrocene)palladium(II) dichloride (Pd(dppf)Cl₂) and tripotassium phosphate (K₃PO₄) or a similar reagent. Alternative catalysts to Pd(dppf)Cl₂ may also be used, such as a suitable palladium catalyst (e.g., a catalyst suitable for a Suzuki reaction). The coupling of A-1 and A-2 may be carried out in a mixture of dioxane and water, or a similar solvent or mixture, and heated to 80° C. or temperature sufficient to provide A-3.

In Step 2, A-5 is prepared by incubating A-3 with A-4 in the presence of 1,1′-bis(diphenylphosphino)ferrocene)palladium(II) dichloride (Pd(dppf)Cl₂) and tripotassium phosphate (K₃PO₄) or a suitable alternative, such as potassium acetate (KOAc). Alternative catalysts to Pd(dppf)Cl₂ might also be used, for example, tris(dibenzylideneacetone)-dipalladium(0) (Pd₂(dba)₃). The reaction can be carried out in dioxane, a mixture of dioxane and water, or a similar solvent or solvent mixture, at 80° C. or another temperature, for example, 60° C. or 100° C., sufficient to provide A-5. In Step 3, A-5 is converted to A-6 by treatment with boron tribromide (BBr₃) or a suitable alternative (e.g., another Lewis acid).

In Step 4, A-6 is reduced to the diol A-7 using sodium borohydride (NaBH₄) or another suitable reductant. A-7 is then cyclized in Step 5 to provide A-8 by treatment of A-7 with diisopropyl azodicarboxylate (DIAD) and triphenylphosphine (PPh₃) using tetrahydrofuran (TIF) or any other suitable solvent. Step 5 may also be carried out with other reagents capable of cyclizing A-7 (e.g., reagents used for a Mitsunobu reaction). For example, alternatives to DIAD may include 1,1′-(azodicarbonyl)dipiperidine (ADDP) and di-tert-butyl azodicarboxylate (TBAD); alternatives to PPh₃ may include tributylphosphene (P(ⁿ⁻Bu)₃).

A-8 is then coupled with A-9 to provide the compound of Formula (I-I) in Step 6. This coupling reaction may be conducted in the presence of [(2-Di-tert-butylphosphino-2′,4′,6′-triisopropyl-1,1′-biphenyl)-2-(2′-amino-1,1′-biphenyl)] palladium(II) methanesulfonate (^{t}BuXPhos-Pd-G3) and NaO^{t}Bu or a similar reagent. As in the previous steps, alternative catalysts to ^{t}BuXPhos-Pd-G3may be used, such as any suitable palladium catalyst, for example, Pd(dppf)Cl₂. Alternative salts to NaO^{t}Bu may be used, such as K₃PO₄ or K₂CO₃. The reaction of Step 6 is conducted in a mixture of dioxane and water, or other suitable solvents, and the mixture is heated to 80° C. or another temperature, for example, 100° C., sufficient to provide the compound of Formula (I-I) or a precursor to a compound of Formula (I-I). A precursor to a compound of Formula (I-I) may be modified to arrive at a compound of Formula (I-I), for example, by removal of protecting groups and/or methylation. Each starting material and/or intermediate in Scheme A may be protected and deprotected using standard protecting group methods. In addition, purification and characterization of each intermediate as well as the final compound of Formula (I) may be afforded by any accepted procedure.

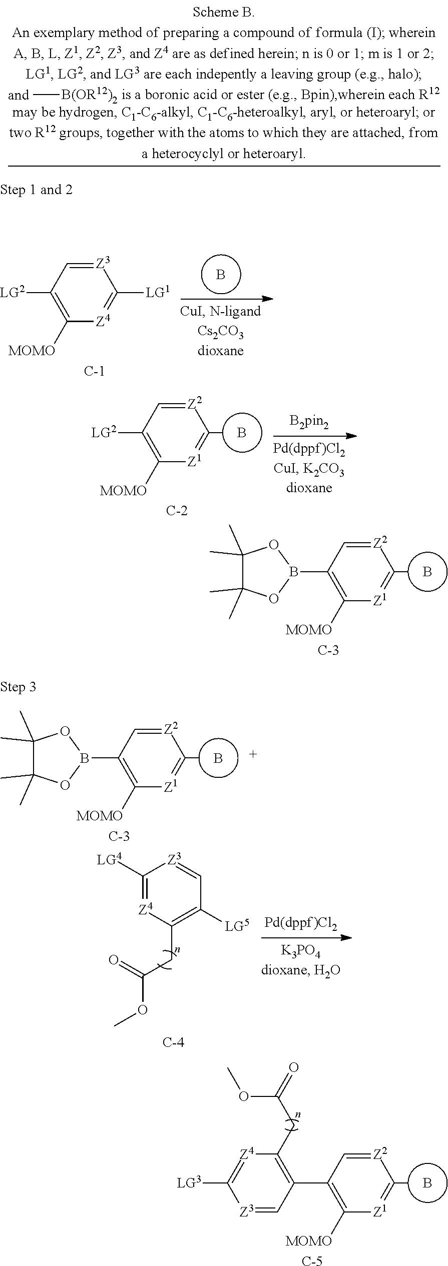

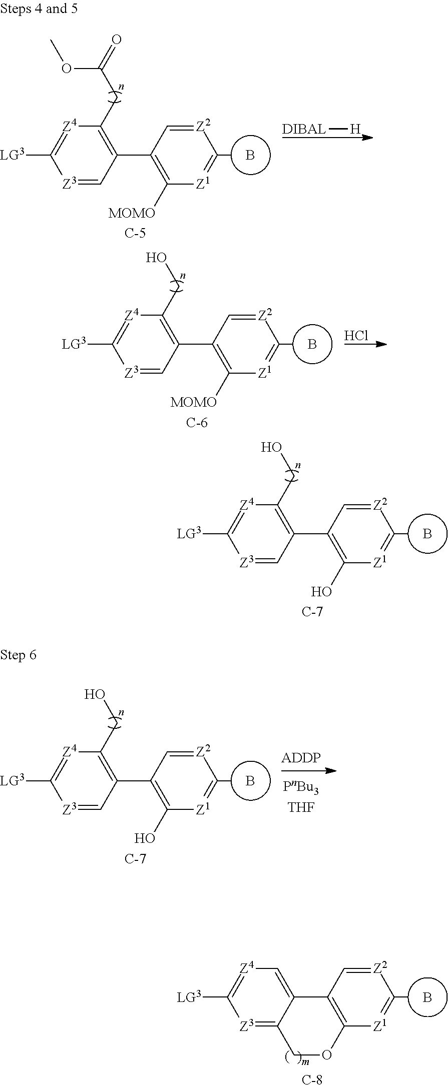

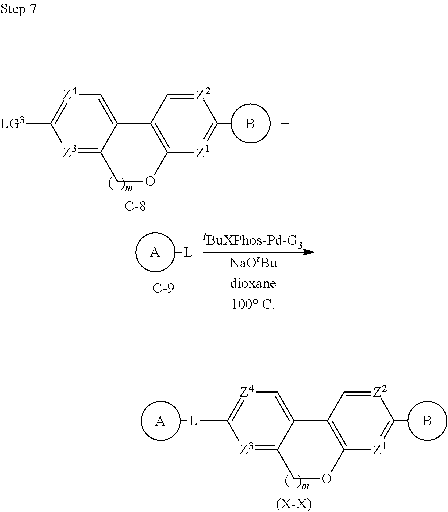

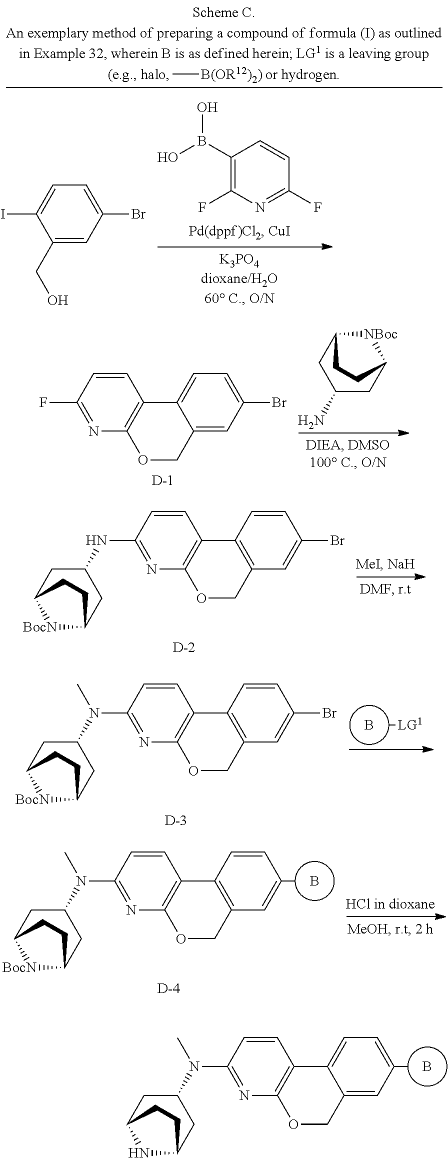

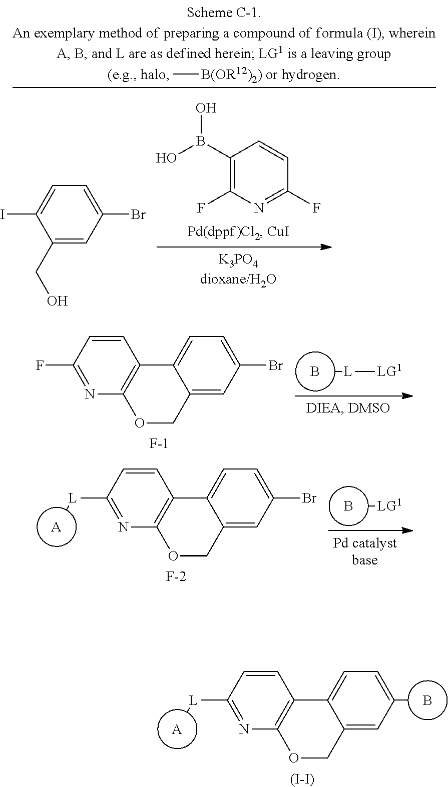

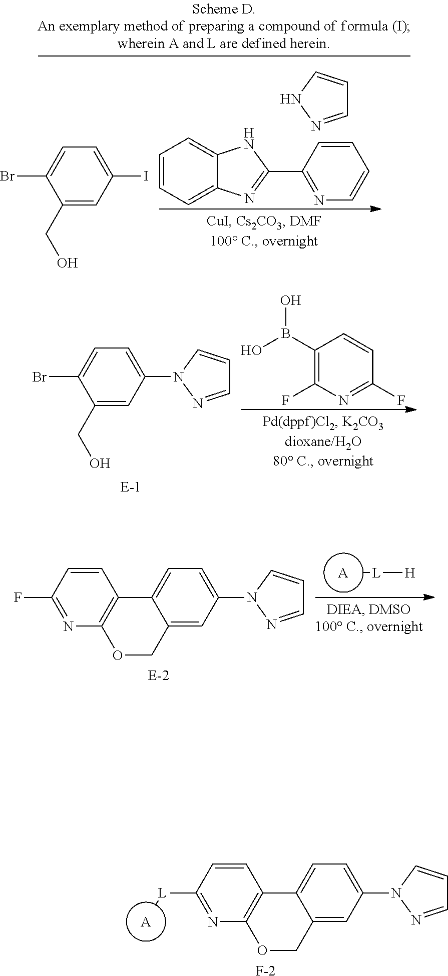

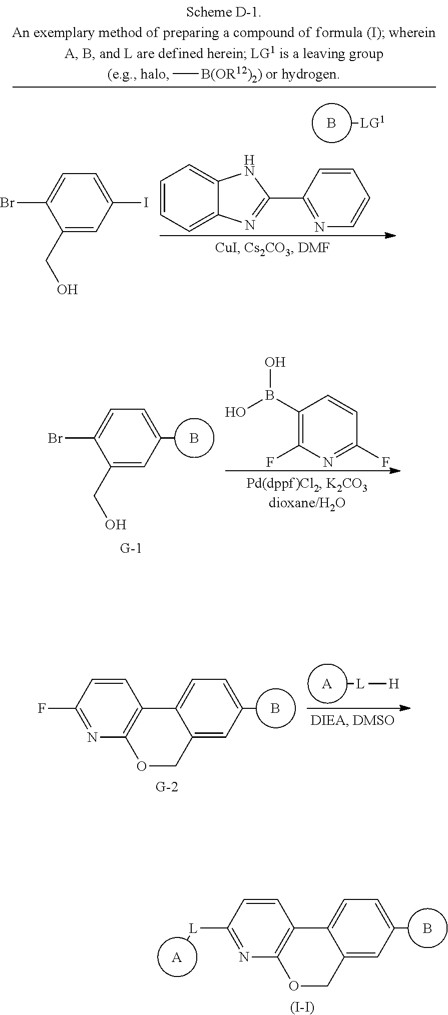

Exemplary synthetic protocols of Compounds 1-216 are outlined in Examples 1-43 in WO 2021/174167, which is incorporated herein by reference in its entirety.

### Example 44: Synthesis of Compound 217

### Synthesis of Intermediate C1

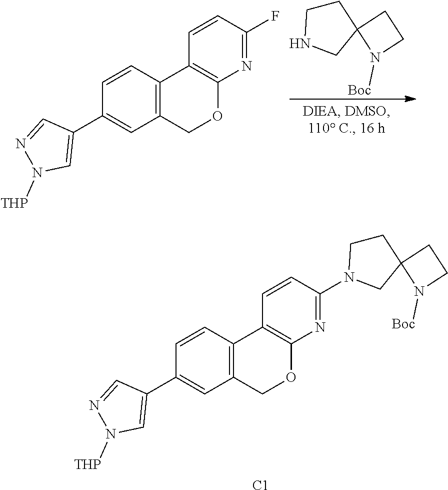

To a stirred mixture of 4-{3-fluoro-6H-isochromeno[3,4-b]pyridin-8-yl}-1-(oxan-2-yl)pyrazole (100.0 mg, 0.285 mmol, 1.0 equiv) and tert-butyl 1,6-diazaspiro[3.4]octane-1-carboxylate, ½ oxalate (73.23 mg, 0.285 mmol, 1.0 equiv) in DMSO (2 mL) was added DIEA (110.3 mg, 0.855 mmol, 3.0 equiv) at room temperature. The resulting mixture was stirred for 16 hr at 110° C. The resulting mixture was diluted with water (4 mL) And extracted with ethyl acetate (2×4 mL). The combined organic layers were washed with water (2×4 mL), dried over anhydrous Na2SO4. After filtration, the filtrate was concentrated under reduced pressure. The residue was purified by silica gel column chromatography, eluted with PE/EA (1:1) to afford tert-butyl 6-{8-[1-(oxan-2-yl)pyrazol-4-yl]-6H-isochromeno[3,4-b]pyridin-3-yl}-1,6-diazaspiro[3.4]octane-1-carboxylate (C1, 130 mg) as an oil that was taken to the next step without further purification. LCMS (ES, m/z): 544 [M+H]⁺

### Synthesis of Compound 217

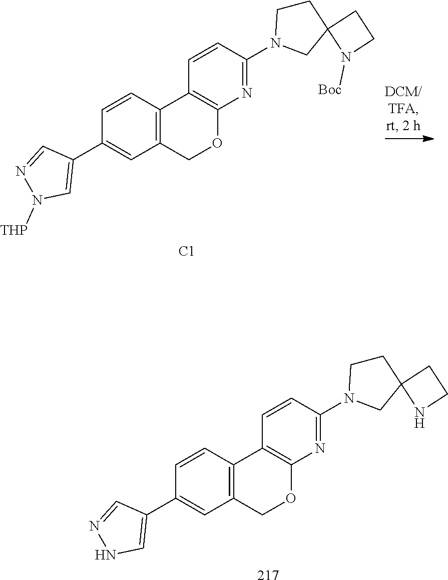

To a stirred mixture of tert-butyl 6-{8-[1-(oxan-2-yl)pyrazol-4-yl]-6H-isochromeno[3,4-b]pyridin-3-yl}-1,6-diazaspiro[3.4]octane-1-carboxylate (120.0 mg, 0.221 mmol, 1.0 equiv) in dichloromethane (2 mL) was added trifluoroacetic acid (1 mL) dropwise at room temperature. The resulting mixture was stirred for 2 hr at room temperature. The resulting mixture was concentrated under reduced pressure. The crude product was purified by Prep-HPLC (Condition 5 Gradient 1) to afford 6-[8-(1H-pyrazol-4-yl)-6H-isochromeno[3,4-b]pyridin-3-yl]-1,6-diazaspiro[3.4]octane (Compound 217, 50 mg, 63%) as a solid. LCMS (ES, m/z): 360 [M+H]^{+ 1}H NMR (400 MHz, DMSO-d₆) δ 8.06-8.04 (m, 2H), 7.98 (d, J=8.5 Hz, 1H), 7.62-7.54 (m, 2H), 7.45 (d, J=1.7 Hz, 1H), 6.17 (d, J=8.4 Hz, 1H), 5.20 (s, 2H), 3.79-3.63 (m, 1H), 3.55-3.45 (m, 2H), 3.43-3.38 (m, 4H), 2.38-2.26 (m, 1H), 2.23-2.18 (m, 1H), 2.09 (t, J=6.9 Hz, 2H).

### Example 45: Synthesis of Compound 219

### Synthesis of Intermediate C2

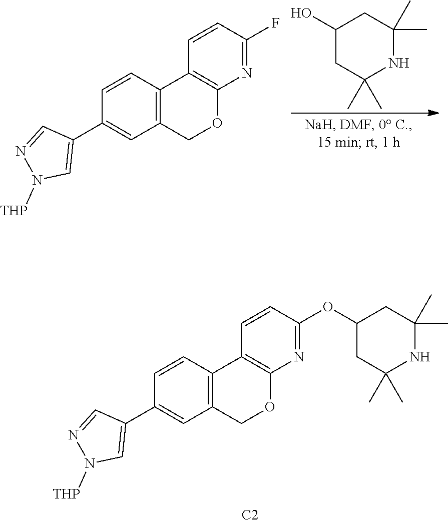

To a stirred solution of 2,2,6,6-tetramethylpiperidin-4-ol (33.6 mg, 0.213 mmol, 1.5 equiv) in DMF (1.0 mL) was added NaH (4.8 mg, 0.199 mmol, 1.4 equiv) in portions at 0° C. The resulting mixture was stirred for 15 min at 0° C. To the above mixture was added 3-fluoro-8-(1-(tetrahydro-2H-pyran-2-yl)-1H-pyrazol-4-yl)-6H-isochromeno[3,4-b] pyridine (50.0 mg, 0.142 mmol, 1.0 equiv) dropwise portions over 2 min at 0° C. The resulting mixture was stirred for additional 1 hr at room temperature. The reaction was quenched with water at 0° C. The resulting mixture Was extracted with ethyl acetate (3×3 mL). The combined organic layers were washed with water (3×6 mL), dried over anhydrous Na₂SO₄. After filtration, the filtrate was concentrated under reduced pressure. The residue was purified by silica gel column chromatography, eluted with CH₂Cl₂/MeOH (5:1) to afford 8-(1-(tetrahydro-2H-pyran-2-yl)-1H-pyrazol-4-yl)-3-((2,2,6,6-tetramethylpiperidin-4-yl)oxy)-6H-isochromeno[3,4-b]pyridine (C2, 65 mg, 74%) as a solid that was taken to the next step without further purification. LCMS (ES, m/z): 489.4 [M+H]⁺

### Synthesis of Compound 219

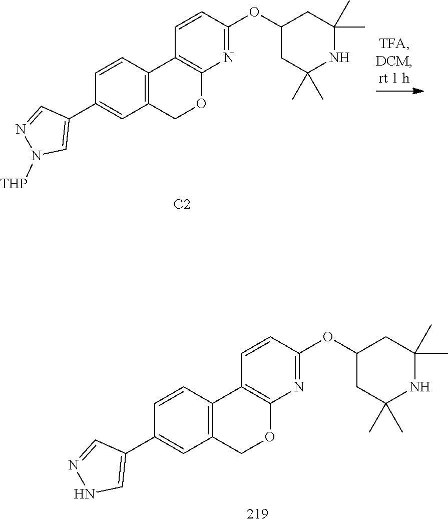

To a stirred solution of 8-(1-(tetrahydro-2H-pyran-2-yl)-1H-pyrazol-4-yl)-3-((2,2,6,6-tetramethylpiperidin-4-yl)oxy)-6H-isochromeno [3,4-b] pyridine (65.0 Mg, 0.133 mmol, 1.0 equiv) in DCM (0.65 mL) was added TFA (0.33 mL) dropwise at room temperature. The resulting mixture was stirred for 30 min at room temperature. The resulting mixture was concentrated under reduced pressure. The residue was neutralized to pH 7 with saturated Na₂CO₃ (aq.). The crude product was purified by Prep-HPLC with the following conditions (Condition 6, Gradient 1) to afford 8-(1H-pyrazol-4-yl)-3-((2,2,6,6-tetramethylpiperidin-4-yl) oxy)-6H-isochromeno [3,4-b] pyridine (Compound 219, 30.3 mg, 56%) as a solid. LCMS (ES, m/z): 405.2 [M+H]+ 1H NMR (300 MHz, DMSO-d₆) δ 8.63 (s, 1H), 8.26 (d, J=8.4 Hz, 1H), 8.10 (s, 2H), 8.05-7.93 (m, 1H), 7.75 (d, J=8.1 Hz, 1H), 7.68-7.59 (m, 1H), 7.52 (d, J=1.8 Hz, 1H), 6.57 (d, J=8.3 Hz, 1H), 5.45-5.36 (m, 1H), 5.34 (s, 2H), 2.28-2.17 (m, 2H), 1.74-1.60 (m, 2H), 1.47 (s, 6H), 1.43 (s, 6H).

### Example 46: Synthesis of Compound 220

### Synthesis of Compound 220

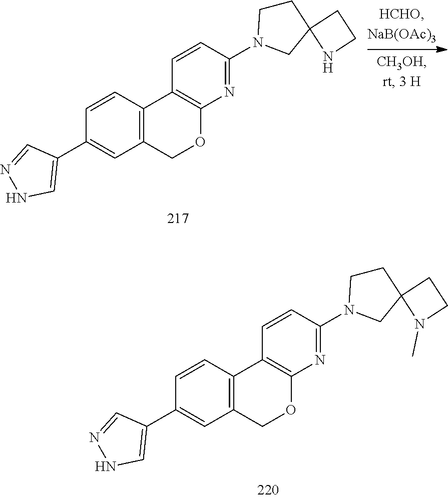 [SMILES: c1cc2c(cc1-c1cn[nH]c1)COc1nc(N3CCC4(CCN4)C3)ccc1-2 ; "217" ; CN1CCC12CCN(c1ccc3c(n1)OCc1cc(-c4cn[nH]c4)ccc1-3)C2]

To a stirred mixture of 6-[8-(1H-pyrazol-4-yl)-6H-isochromeno[3,4-b]pyridin-3-yl]-1,6-diazaspiro[3.4]octane (40 mg, 0.111 mmol, 1.0 equiv) and methanal (20.0 mg, 0.666 mmol, 6.0 equiv) in methanol (2.0 mL) was added NaBH(OAc)₃ (70.7 mg, 0.333 mmol, 3.0 equiv) at room temperature. The resulting mixture was stirred for 2 hr at room temperature. The resulting mixture was concentrated under reduced pressure. The crude product was purified by Prep-HPLC (Condition 5, Gradient 1) to afford 3-(1-methyl-1,6-diazaspiro[3.4]octan-6-yl)-8-(1H-pyrazol-4-yl)-6H-isochromeno[3,4-b]pyridine (Compound 220, 27.9 mg, 67%) as a solid. LCMS (ES, m/z): 374 [M+H]+¹H NMR (400 MHz, DMSO-d₆) δ 12.93 (s, 1H), 8.11-8.04 (m, 2H), 7.99 (d, J=8.4 Hz, 1H), 7.62-7.55 (m, 2H), 7.45 (d, J=1.7 Hz, 1H), 6.20 (d, J=8.4 Hz, 1H), 5.21 (s, 2H), 3.55-3.47 (m, 2H), 3.37-3.34 (m, 1H), 3.13 (q, J=6.2 Hz, 1H), 3.03 (q, J=6.9 Hz, 1H), 2.14 (s, 3H), 2.12-1.88 (m, 4H).

### Example 47: Synthesis of Compound 221

### Synthesis of Intermediate C3

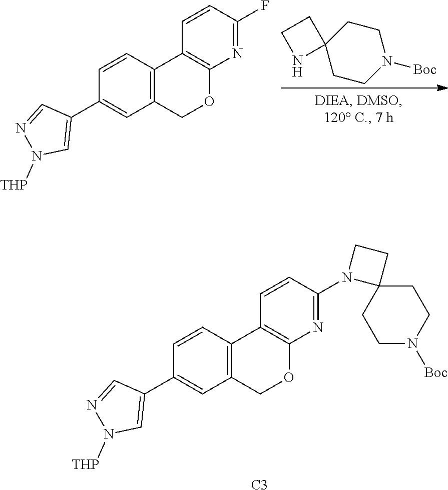

To a stirred mixture of 4-{3-fluoro-6H-isochromeno[3,4-b]pyridin-8-yl}-1-(oxan-2-yl)pyrazole (150.0 mg, 0.427 mmol, 1.0 equiv) and tert-butyl 1,7-diazaspiro[3.5]nonane-7-carboxylate (96.6 Mg, 0.427 mmol, 1.0 equiv) in DMSO (1.5 mL) was added DIEA (165.5 mg, 1.281 mmol, 3.0 equiv) at room temperature. The resulting mixture was stirred for 7 hr at 120° C. The resulting mixture was diluted with water (4 mL). The resulting Mixture was extracted with EtOAc (2×5 mL). The combined organic layers were washed with water (1×7 mL) and dried over anhydrous Na₂SO₄. After filtration, the filtrate was concentrated under reduced pressure. The residue was purified by silica gel column chromatography, eluted with PE/EA (1:1) to afford tert-butyl 1-{8-[1-(oxan-2-yl)pyrazol-4-yl]-6H-isochromeno[3,4-b]pyridin-3-yl}-1,7-diazaspiro[3.5]nonane-7-carboxylate (C3, 140 mg, 58%) as an oil that was taken to the next step without further purification. LCMS (ES, m/z): 558 [M+H]⁺

### Synthesis of Compound 221

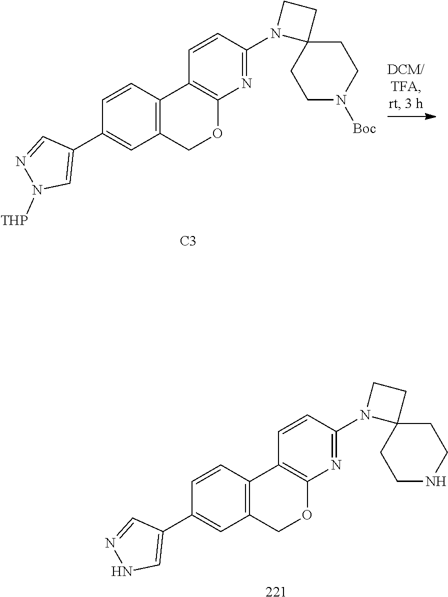

To a stirred mixture of tert-butyl 1-{8-[1-(oxan-2-yl)pyrazol-4-yl]-6H-isochromeno[3,4-b]pyridin-3-yl}-1,7-diazaspiro[3.5]nonane-7-carboxylate (130.0 mg, 0.233 mmol, 1.0 equiv) in DCM (2 mL) was added TFA (1 mL) dropwise at room temperature. The resulting mixture was stirred for 3 hr at room temperature. The resulting mixture was concentrated under reduced pressure. The crude product was purified by Prep-HPLC (Condition 5, Gradient 2) to afford 1-[8-(1H-pyrazol-4-yl)-6H-isochromeno[3,4-b]pyridin-3-yl]-1,7-diazaspiro[3.5]nonane 2,2,2-trifluoroacetate (Compound 22153 mg, 46%) as a solid. LCMS (ES, m/z): 374 [M+H]+¹H NMR (400 MHz, DMSO-d₆) δ 12.92 (s, 1H), 8.53-8.51 (m, 1H), 8.35-8.33 (m, 1H), 8.17-7.90 (m, 3H), 7.63 (d, J=8.1 Hz, 1H), 7.57 (dd, J=8.0, 1.8 Hz, 1H), 7.45 (d, J=1.8 Hz, 1H), 6.03 (d, J=8.3 Hz, 1H), 5.22 (s, 2H), 3.84 (t, J=7.3 Hz, 2H), 3.36-3.35 (m, 2H), 2.91 (t, J=12.3 Hz, 2H), 2.64-2.58 (m, 2H), 2.24 (t, J=7.3 Hz, 2H), 1.91 (d, J=13.3 Hz, 2H).

### Example 48: Synthesis of Compound 222

### Synthesis of Compound 222

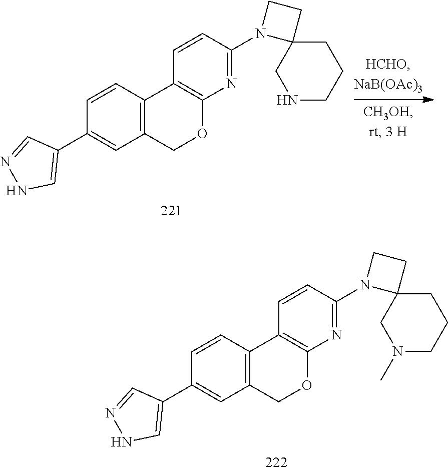 [SMILES: c1cc2c(cc1-c1cn[nH]c1)COc1nc(N3CCC34CCCNC4)ccc1-2 ; "221" ; CN1CCCC2(CCN2c2ccc3c(n2)OCc2cc(-c4cn[nH]c4)ccc2-3)C1 ; "222"]

To a stirred mixture of 1-[8-(1H-pyrazol-4-yl)-6H-isochromeno[3,4-b]pyridin-3-yl]-1,6-diazaspiro[3.5]nonane (30.0 mg, 0.080 mmol, 1.0 equiv) and methanal (14.5 MG, 0.480 mmol, 6.0 equiv) in CH₃OH (2.5 mL) was added NaBH(OAc)₃ (51.1 mg, 0.240 mmol, 3.0 equiv) at room temperature. The resulting mixture was stirred for 3 hr at room temperature. The reaction was quenched with water at room temperature. The mixture was purified by Prep-HPLC (Condition 5, Gradient 3) to afford 6-methyl-1-[8-(1H-pyrazol-4-yl)-6H-isochromeno[3,4-b]pyridin-3-yl]-1,6-diazaspiro[3.5]nonane (Compound 222, 5.2 mg, 16%) as a solid. LCMS (ES, m/z): 388 [M+H]+¹H NMR (400 MHz, DMSO-d₆) δ 12.92 (s, 1H), 8.18-8.02 (m, 2H), 7.94 (d, J=8.4 Hz, 2H), 7.64-7.51 (m, 2H), 7.45 (d, J=1.6 Hz, 1H), 6.04 (d, J=8.3 Hz, 1H), 5.20 (s, 2H), 3.80 (dd, J=8.5, 6.6 Hz, 2H), 2.79 (d, J=10.3 Hz, 1H), 2.68-2.65 (m, 1H), 2.59-2.57 (m, 1H), 2.23-2.16 (m, 4H), 2.15-2.01 (m, 2H), 1.89-1.70 (m, 2H), 1.63-1.60 (m, 1H), 1.55-1.44 (m, 1H).

### Example 49: Synthesis of Compound 223

### Synthesis of Intermediate C4

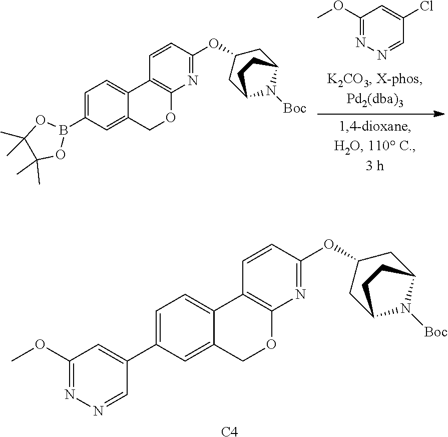

To a solution of tert-butyl (1R,3R,5S)-3-{[8-(4,4,5,5-tetramethyl-1,3,2-dioxaborolan-2-yl)-6H-isochromeno[3,4-b]pyridin-3-yl]oxy}-8-azabicyclo[3.2.1]octane-8-carboxylate (80 mg, 0.150 mmol, 1.00 equiv) and 5-chloro-3-methoxypyridazine (32.46 mg, 0.225 mmol, 1.50 equiv) in 1,4-dioxane (1.0 mL, 11.350 mmol, 75.83 equiv) and H₂O (0.2 mL, 11.102 mmol, 74.17 equiv) were added K₂CO₃ (62.06 mg, 0.450 mmol, 3.0 equiv), X-phos (7.14 mg, 0.015 mmol, 0.1 equiv) and Pd₂(dba)₃ (13.71 mg, 0.015 mmol, 0.1 equiv) at room temperature under nitrogen atmosphere. The resulting mixture was stirred for 3 hr at 110° C. under nitrogen atmosphere. The mixture was diluted with ethyl acetate (10 ml), dried over Na₂SO₄, and concentrated under reduced pressure. The residue was purified by silica gel column chromatography, eluted with PE/EA (5:1) to afford tert-butyl (1R,3R,5S)-3-{[8-(6-methoxypyridazin-4-yl)-6H-isochromeno[3,4-b]pyridin-3-yl]oxy}-8-azabicyclo[3.2.1]octane-8-carboxylate (C4, 75 mg, 96%) as a solid that was taken to the next step without further purification. LCMS (ES, m/z): 517 [M+H]⁺

### Synthesis of Compound 223

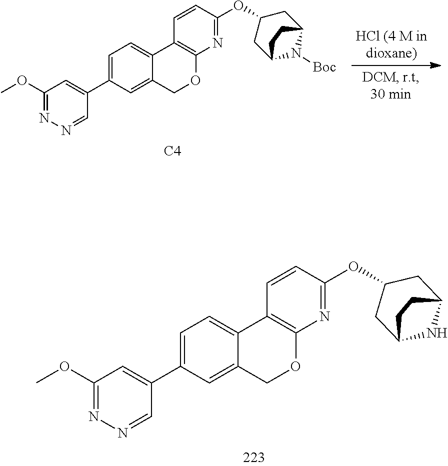

To a stirred solution of tert-butyl (1R,3R,5S)-3-{[8-(6-methoxypyridazin-4-yl)-6H-isochromeno[3,4-b]pyridin-3-yl]oxy}-8-azabicyclo[3.2.1]octane-8-carboxylate (75 mg, 0.145 mmol, 1.00 equiv) in DCM (2 mL) was added a solution of hydrochloric acid in 1,4-dioxane (1 mL, 32.912 mmol, 226.70 equiv) dropwise at room temperature. The resulting mixture was stirred for 30 min at room temperature. The resulting mixture was concentrated under reduced pressure. The crude product was purified by Prep-HPLC (Condition 7, Gradient 1) to afford (1R,3R,5S)-3-{[8-(6-methoxypyridazin-4-yl)-6H-isochromeno[3,4-b]pyridin-3-yl]oxy}-8-azabicyclo[3.2.1]octane (Compound 223, 15 mg, 24%) as a solid. LCMS (ES, m/z): 417 [M+H]+ 1H NMR (400 MHz, DMSO-d6) δ 9.35 (d, J=1.9 Hz, 1H), 8.30 (d, J=8.4 Hz, 1H), 7.97-7.87 (m, 3H), 7.54 (d, J=1.9 Hz, 1H), 6.52 (d, J=8.3 Hz, 1H), 5.39 (s, 2H), 5.22 (tt, J=10.8, 5.7 Hz, 1H), 4.09 (s, 3H), 3.49 (d, J=4.4 Hz, 2H), 2.09-1.99 (m, 2H), 1.75-1.62 (m, 4H), 1.58-1.47 (m, 2H).

### Experiment 50: Synthesis of Compound 224

### Synthesis of Intermediate CS

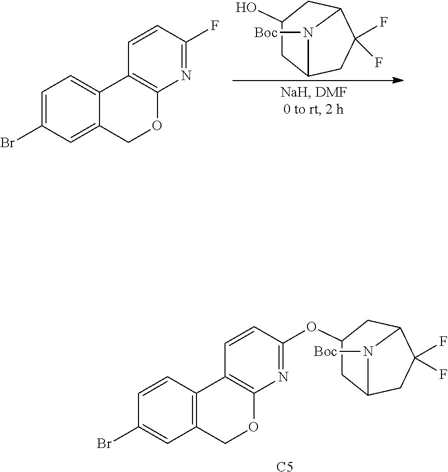

To a solution of tert-butyl 6,6-difluoro-3-hydroxy-8-azabicyclo[3.2.1]octane-8-carboxylate (235.00 mg, 0.893 mmol, 1 equiv) in DMF (2.3 mL) was added sodium hydride (60% in oil, 53 mg) at 0° C. The mixture was stirred for 15 min. 8-bromo-3-fluoro-6H-isochromeno[3,4-b]pyridine (250 mg, 0.893 mmol, 1.00 equiv) was added and the mixture was allowed to warm to room temperature and stirred for 2 hr. The resulting mixture was quenched with water (10 mL) extracted with ethyl acetate (20 mL×2). The combined organic layers were washed with H₂O (20 mL×2), dried over anhydrous Na2SO4 and concentrated under reduced pressure. The residue was purified by silica gel column chromatography, eluted with PE/EA (1:1) to afford tert-butyl 3-({8-bromo-6H-isochromeno[3,4-b]pyridin-3-yl}oxy)-6,6-difluoro-8-azabicyclo[3.2.1]octane-8-carboxylate (C5, 440 mg, 94%) as a solid that was taken to the next step without further purification. LCMS (ES, m/z): 523[M+H]⁺

### Synthesis of Intermediate C6

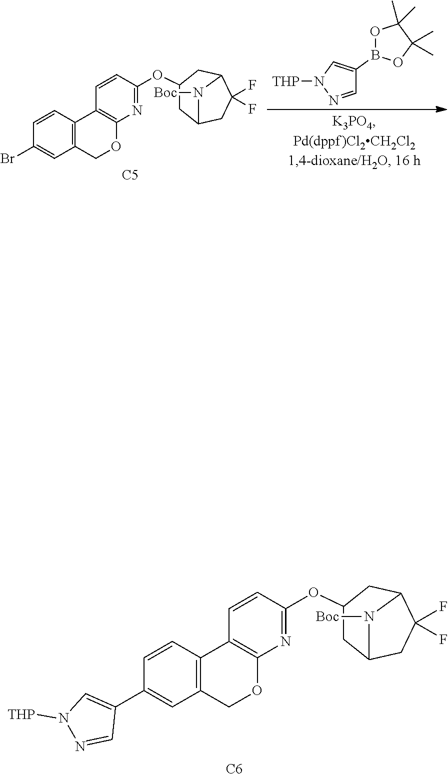

To a solution of tert-butyl-3-({8-bromo-6H-isochromeno[3,4-b]pyridin-3-yl}oxy)-6,6-difluoro-8-azabicyclo[3.2.1]octane-8-carboxylate (100 mg, 0.019 mmol, 1.00 equiv) and 1-(oxan-2-yl)-4-(4,4,5,5-tetramethyl-1,3,2-dioxaborolan-2-yl)pyrazole (55.80 mg, 0.201 mmol, 1.5 equiv) in 1,4-dioxane (1.0 mL)/H₂O (0.2 mL) was added K₃PO₄ (85.17 mg, 0.402 mmol, 3 equiv) and Pd(dppf)Cl₂·CH₂Cl₂ (10.90 mg, 0.013 mmol, 0.1 equiv). After stirring for 16 hr at 100° C. under nitrogen atmosphere, the resulting mixture was concentrated under reduced pressure. The residue was purified by silica gel column chromatography, eluted with PE/EA (5:1) to afford tert-butyl-6,6-difluoro-3-({8-[1-(oxan-2-yl)pyrazol-4-yl]-6H-isochromeno[3,4-b]pyridin-3-yl}oxy)-8-azabicyclo[3.2.1]octane-8-carboxylate as a solid that was taken to the next step without further purification. LCMS (ES, m/z): 595[M+H]⁺

### Synthesis of Compound 224

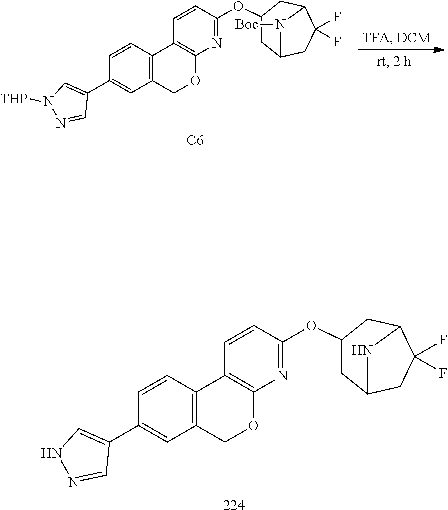

To a solution of tert-butyl 6,6-difluoro-3-({8-[1-(oxan-2-yl)pyrazol-4-yl]-6H-isochromeno[3,4-b]pyridin-3-yl}oxy)-8-azabicyclo[3.2.1]octane-8-carboxylate (75 MG, 0.126 mmol, 1.00 equiv) in DCM (0.75 mL, 11.798 mmol, 93.54 equiv) was added TFA (0.75 mL, 10.097 mmol, 80.06 equiv) dropwise at room temperature. The reaction mixture was stirred for 2 hr at room temperature. The resulting mixture was concentrated under reduced pressure. The crude product was purified by Prep-HPLC (Condition 7, Gradient 2) to afford 6,6-difluoro-3-{[8-(1H-pyrazol-4-yl)-6H-isochromeno[3,4-b]pyridin-3-yl]oxy}-8-azabicyclo[3.2.1]octane (Compound 224, 30.2 mg, 58%) as a solid. LCMS (ES, m/z): 411[M+H]^{+ 1}H NMR (400 MHz, DMSO-d6) δ 12.97 (s, 1H), 8.21 (d, J=8.9 Hz, 2H), 7.98-7.93 (m, 1H), 7.72 (d, J=8.1 Hz, 1H), 7.62 (dd, J=8.1, 1.8 Hz, 1H), 7.52 (d, J=1.8 Hz, 1H), 6.50 (d, J=8.3 Hz, 1H), 5.32 (s, 3H), 3.60 (d, J=7.3 Hz, 1H), 3.45 (d, J=13.1 Hz, 1H), 2.88 (s, 1H), 2.35 (dd, J=27.5, 6.7 Hz, 1H), 2.29 (s, 1H), 2.16 (t, J=12.8 Hz, 2H), 1.62 (dt, J=34.0, 11.1 Hz, 2H).

### Example 51: Synthesis of Compound 225

### Synthesis of Intermediate C7

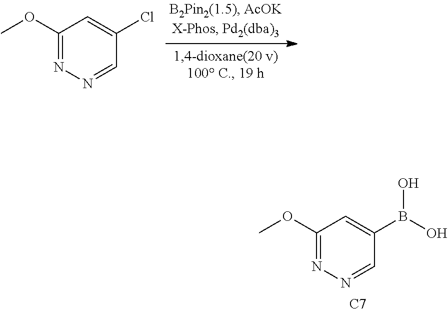

To a stirred mixture of 5-chloro-3-methoxypyridazine (70 mg, 0.484 mmol, 1.00 equiv) and bis(pinacolato)diboron (184.45 mg, 0.726 mmol, 1.5 equiv) in 1,4-dioxane (2 mL) were added potassium acetate (142.57 mg, 1.452 mmol, 3 equiv), X-Phos (23.08 mg, 0.048 mmol, 0.1 equiv) and Pd₂(dba)₃ (44.34 mg, 0.048 mmol, 0.1 equiv) at room temperature under nitrogen atmosphere. The resulting mixture was stirred for 19 hr at 100° C. under nitrogen atmosphere. The resulting mixture was used in the next step directly without further purification. LCMS (ES, m/z): 155[M+H]⁺

### Synthesis of Intermediate C8

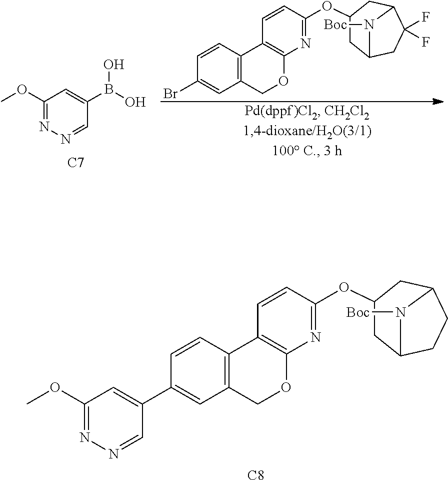

To the mixture of C7 was added tert-butyl 3-({8-bromo-6H-isochromeno[3,4-b]pyridin-3-yl}oxy)-6,6-difluoro-8-azabicyclo[3.2.1]octane-8-carboxylate (224.41 mg, 0.429 mmol, 1.1 equiv), H₂O (0.7 mL) and Pd(dppf)Cl₂·CH₂Cl₂ (31.75 mg, 0.039 mmol, 0.1 equiv) at room temperature under nitrogen atmosphere. After stirring for 3 hr at 100° C. under nitrogen atmosphere, the resulting mixture was concentrated under reduced pressure. The residue was purified by silica gel column chromatography, eluted with PE/EA (5:1) to afford tert-butyl 6,6-difluoro-3-{[8-(6-methoxypyridazin-4-yl)-6H-isochromeno[3,4-b]pyridin-3-yl]oxy}-8-azabicyclo[3.2.1]octane-8-carboxylate (C8, 83 mg, 38%) as a solid that was taken to the next step without further purification. LCMS (ES, m/z): 553[M+H]⁺

### Synthesis of Compound 225

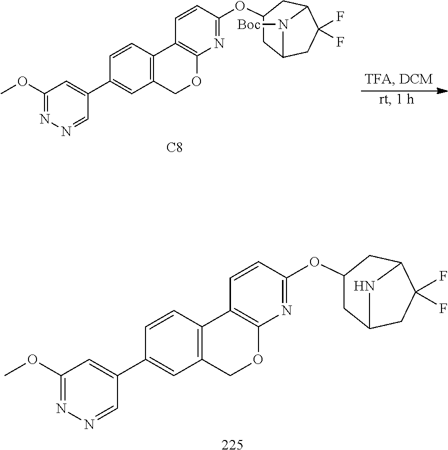

To a solution of tert-butyl 6,6-difluoro-3-{[8-(6-methoxypyridazin-4-yl)-6H-isochromeno[3,4-b]pyridin-3-yl]oxy}-8-azabicyclo[3.2.1]octane-8-carboxylate (83 Mg, 0.150 mmol, 1.00 equiv) in DCM (0.8 mL, 12.584 mmol, 83.78 equiv) was added TFA (0.8 mL, 10.770 mmol, 71.70 equiv) at room temperature. The mixture was stirred for 1 h at room temperature and concentrated under reduced pressure. The residue was purified by Prep-HPLC (Condition 7, Gradient 3) to afford 6,6-difluoro-3-{[8-(6-methoxypyridazin-4-yl)-6H-isochromeno[3,4-b]pyridin-3-yl]oxy}-8-azabicyclo[3.2.1]octane (Compound 225, 21.8 mg, 32%) as a solid. LCMS (ES, m/z): 453[M+H]^{+ 1}H NMR (400 MHz, DMSO-d₆) δ 9.35 (d, J=1.8 Hz, 1H), 8.32 (d, J=8.5 Hz, 1H), 7.98-7.87 (m, 3H), 7.55 (t, J=1.6 Hz, 1H), 6.58-6.51 (m, 1H), 5.42-5.29 (m, 3H), 4.09 (d, J=1.5 Hz, 3H), 3.60 (s, 1H), 3.45 (d, J=13.0 Hz, 1H), 2.89 (s, 1H), 2.44-2.22 (m, 3H), 2.17 (t, J=12.4 Hz, 2H), 1.63 (dt, J=34.3, 11.6 Hz, 2H).

### Example 52: Synthesis of Compound 226

### Synthesis of Compound 226

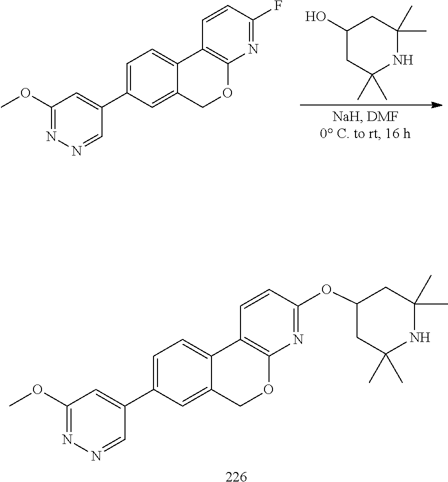

To a solution of 2,2,6,6-tetramethylpiperidin-4-ol (30.51 mg, 0.194 mmol, 1.5 equiv) in DMF (0.8 mL, 10.337 mmol, 79.93 equiv) was added sodium hydride (60% in oil, 7.24 mg) at 0° C. The mixture was stirred for 15 min. 5-{3-fluoro-6H-isochromeno[3,4-b]pyridin-8-yl}-3-methoxypyridazine (40 mg, 0.129 mmol, 1.00 equiv) was added and the mixture was allowed to warm to room temperature and stirred for 16 hr. The resulting mixture was quenched with water (0.1 mL) and purified by Prep-HPLC (Condition 7, Gradient 1) to afford 3-methoxy-5-{3-[(2,2,6,6-tetramethylpiperidin-4-yl)oxy]-6H-isochromeno[3,4-b]pyridin-8-yl}pyridazine (Compound 226, 5.2 mg, 9%) as a solid. LCMS (ES, m/z): 447[M+H]^{+ 1}H NMR (400 MHz, DMSO-d6) δ 9.35 (d, J=1.9 Hz, 1H), 8.31 (d, J=8.4 Hz, 1H), 7.99-7.86 (m, 3H), 7.55 (d, J=1.9 Hz, 1H), 6.54 (d, J=8.3 Hz, 1H), 5.41 (s, 2H), 4.09 (s, 3H), 1.97 (d, J=10.9 Hz, 2H), 1.21 (s, 7H), 1.10 (s, 6H).

### Example 52: Synthesis of Compound 227

### Synthesis of Compound 227

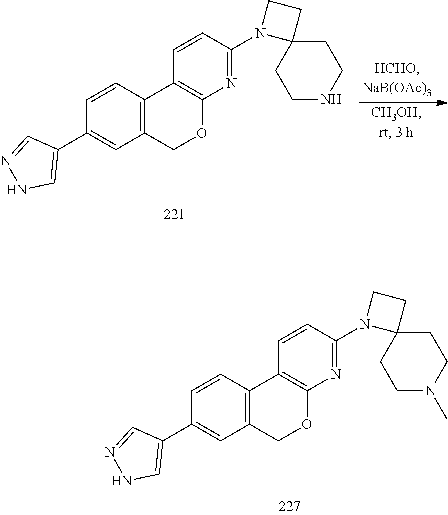 [SMILES: c1cc2c(cc1-c1cn[nH]c1)COc1nc(N3CCC34CCNCC4)ccc1-2 ; CN1CCC2(CC1)CCN2c1ccc2c(n1)OCc1cc(-c3cn[nH]c3)ccc1-2]

To a stirred mixture of 1-[8-(1H-pyrazol-4-yl)-6H-isochromeno[3,4-b]pyridin-3-yl]-1,7-diazaspiro[3.5]nonane (50.0 mg, 0.134 mmol, 1.0 equiv) and HCHO (20.1 mg, 0.670 mmol, 5.0 equiv) in CH₃OH (6 mL) was added NaBH(OAc)₃ (85.1 mg, 0.402 mmol, 3.0 equiv) at room temperature. The resulting mixture was stirred for 16 hr at room temperature. The reaction was quenched with water at room temperature. The crude product was purified by Prep-HPLC (Condition 6, Gradient 2) to afford 7-methyl-1-[8-(1H-pyrazol-4-yl)-6H-isochromeno[3,4-b]pyridin-3-yl]-1,7-diazaspiro[3.5]nonane (Compound 227, 7.5 mg, 14%) as a solid. LCMS (ES, m/z): 388 [M+H]^{+ 1}H NMR (400 MHz, DMSO-d₆) δ 9.36 (s, 1H), 8.17-8.14 (m, 1H), 8.03 (d, J=8.4 Hz, 1H), 7.96-7.91 (m, 1H), 7.63 (d, J=8.1 Hz, 1H), 7.58 (d, J=1.7 Hz, 1H), 7.45 (s, 1H), 6.02 (d, J=8.3 Hz, 1H), 5.23 (s, 2H), 3.84 (t, J=7.3 Hz, 2H), 3.44 (d, J=12.3 Hz, 2H), 3.05-2.98 (m, 2H), 2.78-2.71 (m, 5H), 2.25 (d, J=7.3 Hz, 2H), 1.95 (d, J=13.4 Hz, 2H).

### Example 53: Synthesis of Compound 230

### Synthesis of Intermediate C9

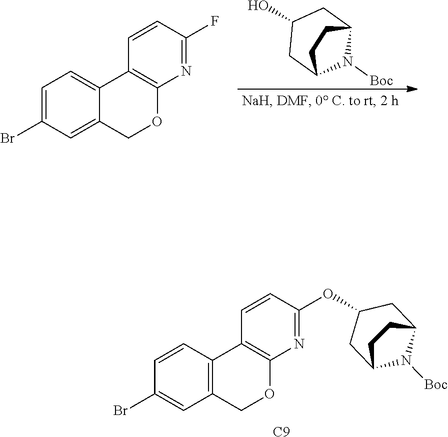

To a solution of tert-butyl (1R,3R,5S)-3-hydroxy-8-azabicyclo[3.2.1]octane-8-carboxylate (267.80 mg, 1.178 mmol, 1.1 equiv) in DMF was added sodium hydride (60% in oil, 60 mg) at 0° C. The mixture was stirred for 15 min. 8-bromo-3-fluoro-6H-isochromeno[3,4-b]pyridine (300 mg, 1.071 mmol, 1.00 equiv) was added and the mixture was allowed to warm to room temperature and stirred for 2 h. The resulting mixture was quenched by water carefully and extracted with EtOAc (2×50 mL). The combined organic layers were washed with brine (2×50 mL), dried over anhydrous Na₂SO₄. After filtration, the filtrate was concentrated under vacuum. The residue was purified by silica gel column chromatography, eluted with PE/EA (2:1) to afford tert-butyl (1R,3R,5S)-3-({8-bromo-6H-isochromeno[3,4-b]pyridin-3-yl}oxy)-8-azabicyclo[3.2.1]octane-8-carboxylate (C9, 400 mg, 76%) as a solid that was taken to the next step without further purification. LCMS (ES, m/z): 487[M+H]⁺

### Synthesis of Intermediate C10

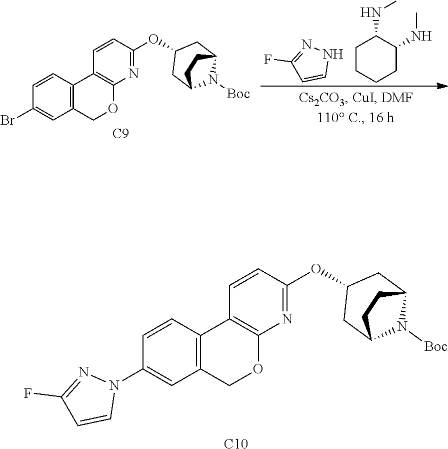

To a stirred mixture of tert-butyl (1R,3R,5S)-3-({8-bromo-6H-isochromeno[3,4-b]pyridin-3-yl}oxy)-8-azabicyclo[3.2.1]octane-8-carboxylate (70 mg, 0.144 mmol, 1.00 equiv) and 3-fluoro-1H-pyrazole (24.72 mg, 0.288 mmol, 2 equiv) in DMF (2 mL) was added Cs₂CO₃ (140.38 mg, 0.432 mmol, 3 equiv), (1R,2S)—N1,N2-dimethylcyclohexane-1,2-diamine (2.04 mg, 0.014 mmol, 0.1 equiv) and CuI (2.74 mg, 0.014 mmol, 0.1 equiv) at room temperature under nitrogen atmosphere. The resulting mixture was stirred for 16 hr at 110° C. under nitrogen atmosphere. The mixture was purified by silica gel column chromatography, eluted with PE/EA (1:1) to afford tert-butyl (1R,3R,5S)-3-{[8-(3-fluoropyrazol-1-yl)-6H-isochromeno[3,4-b]pyridin-3-yl]oxy}-8-azabicyclo[3.2.1]octane-8-carboxylate (C10, 70 mg, 98%) as a solid that was taken to the next step without further purification. LCMS (ES, m/z): 493[M+H]⁺

### Synthesis of Compound-230

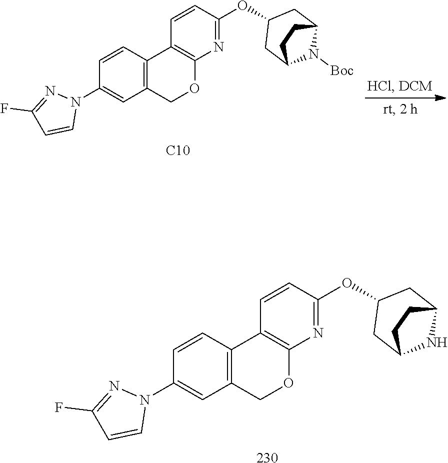

To a stirred solution of tert-butyl (1R,3R,5S)-3-{[8-(3-fluoropyrazol-1-yl)-6H-isochromeno[3,4-b]pyridin-3-yl]oxy}-8-azabicyclo[3.2.1]octane-8-carboxylate (70 mg, 0.142 mmol, 1.00 equiv) in DCM (3 mL) was added a solution of HCl in 1,4-dioxane (1 mL, 32.912 mmol, 231.58 equiv) at room temperature under nitrogen atmosphere. The resulting mixture was stirred for 2 h at room temperature under nitrogen atmosphere. The resulting mixture was concentrated under reduced pressure. The crude product was purified by Prep-HPLC (Condition 7, Gradient 1) to afford (1R,3R,5S)-3-{[8-(3-fluoropyrazol-1-yl)-6H-isochromeno[3,4-b]pyridin-3-yl]oxy}-8-azabicyclo[3.2.1]octane (Compound 230, 25 mg, 40%) as a solid. LCMS (ES, m/z): 393[M+H]+ 1H NMR (300 MHz, DMSO-d6) δ 8.49 (t, J=2.6 Hz, 1H), 8.23 (d, J=8.4 Hz, 1H), 7.86 (d, J=8.5 Hz, 1H), 7.76 (dd, J=8.5, 2.3 Hz, 1H), 7.70 (d, J=2.2 Hz, 1H), 6.50 (d, J=8.3 Hz, 1H), 6.37 (dd, J=5.8, 2.7 Hz, 1H), 5.37 (s, 2H), 5.21 (dq, J=10.8, 5.7, 5.3 Hz, 1H), 3.49 (s, 2H), 2.06-1.97 (m, 1H), 1.67 (dd, J=11.0, 7.6 Hz, 4H), 1.52 (t, J=10.7 Hz, 2H).

### Example 54: Synthesis of Compound 231

### Synthesis of Intermediate C11

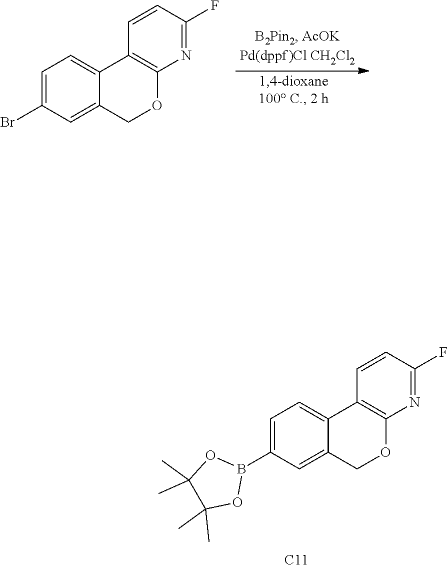 [SMILES: Fc1ccc2c(n1)OCc1cc(Br)ccc1-2 ; CC1(C)OB(c2ccc3c(c2)COc2nc(F)ccc2-3)OC1(C)C ; "C11"]

To a stirred mixture of 8-bromo-3-fluoro-6H-isochromeno[3,4-b]pyridine (600 mg, 2.142 mmol, 1.00 equiv) and bis(pinacolato)diboron (815.96 mg, 3.213 mmol, 1.5 equiv) in 1,4-dioxane was added potassium acetate (420.46 mg, 4.284 mmol, 2 equiv) and Pd(dppf)Cl₂·CH₂C12 (174.50 mg, 0.214 mmol, 0.1 equiv) at room temperature under nitrogen atmosphere. The resulting mixture was stirred for 2 h at 100° C. under nitrogen atmosphere. The resulting mixture was concentrated under vacuum. The residue was purified by silica gel column chromatography, eluted with PE/EA (5:1) to afford 3-fluoro-8-(4,4,5,5-tetramethyl-1,3,2-dioxaborolan-2-yl)-6H-isochromeno[3,4-b]pyridine (C11, 380 mg, 49%) as a solid that was taken to the next step without further purification. LCMS (ES, m/z): 328H]⁺

### Synthesis of Intermediate C12

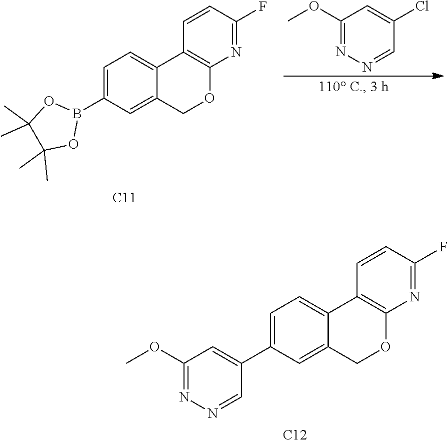

To a stirred mixture of 3-fluoro-8-(4,4,5,5-tetramethyl-1,3,2-dioxaborolan-2-yl)-6H-isochromeno[3,4-b]pyridine (360 mg, 1.100 mmol, 1.00 equiv) and 5-chloro-3-methoxypyridazine (190.88 mg, 1.320 mmol, 1.2 equiv) in 1,4-dioxane (3 mL)/H₂O (1 mL) were added K₂CO₃ (456.24 mg, 3.300 mmol, 3 equiv), X-phos (52.46 mg, 0.110 mmol, 0.1 equiv) and Pd₂(dba)₃ (100.76 mg, 0.110 mmol, 0.1 equiv) at room temperature. The resulting mixture was stirred for 3 hr at 110° C. under nitrogen atmosphere. The resulting mixture was concentrated under reduced pressure. The residue was purified by silica gel column chromatography, eluted with PE/THF (1:1) to afford 5-{3-fluoro-6H-isochromeno[3,4-b]pyridin-8-yl}-3-methoxypyridazine (C12, 124 mg, 36%) as a solid that was taken to the next step without further purification. LCMS (ES, m/z): 310H]⁺

### Synthesis of Compound 231

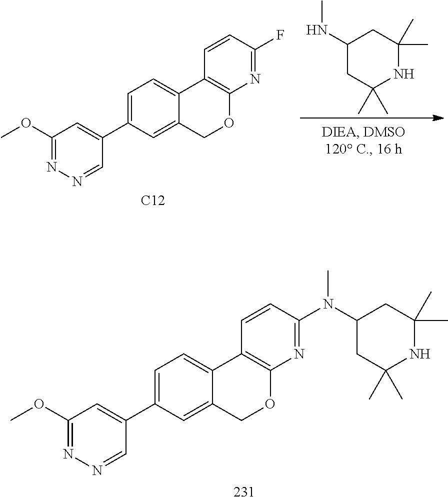

To a stirred mixture of 5-{3-fluoro-6H-isochromeno[3,4-b]pyridin-8-yl}-3-methoxypyridazine (65 mg, 0.210 mmol, 1.00 equiv) and N,2,2,6,6-pentamethylpiperidin-4-amine (42.95 Mg, 0.252 mmol, 1.2 equiv) in DMSO (0.6 mL, 8.447 mmol, 40.20 equiv) were added DIEA (54.32 mg, 0.420 mmol, 2.0 equiv) dropwise at room temperature. The resulting mixture was stirred for 16 h at 120° C. The resulting mixture was purified by Prep-HPLC with the following conditions (Column: XBride Prep OBD Column 19×150 mm 8 um; Mobile Phase A: water (0.05% Nh₃·H₂O), Mobile Phase B: ACN; Flow rate: 20 mL/min; Gradient: 25% B to 50% B in 8 min, 48% B; Wave Length: 220 nm; RT1(min): 7.02) to afford N-[8-(6-methoxypyridazin-4-yl)-6H-isochromeno[3,4-b]pyridin-3-yl]-N,2,2,6,6-pentamethylpiperidin-4-amine (Compound 231, 10 mg, 10%) as a solid. LCMS (ES, m/z): 460[M+H]+¹H NMR (400 MHz, DMSO-d6) δ 9.34 (d, J=1.9 Hz, 1H), 8.11 (d, J=8.7 Hz, 1H), 7.90 (dd, J=8.1, 2.0 Hz, 1H), 7.83 (d, J=1.9 Hz, 1H), 7.78 (d, J=8.3 Hz, 1H), 7.51 (d, J=1.9 Hz, 1H), 6.42 (d, J=8.6 Hz, 1H), 5.30 (s, 2H), 4.91 (s, 1H), 4.09 (s, 3H), 2.86 (s, 3H), 1.51-1.35 (m, 4H), 1.25 (s, 6H), 1.10 (s, 6H).

### Example 55: Synthesis of Compound 232

### Synthesis of Intermediate C13

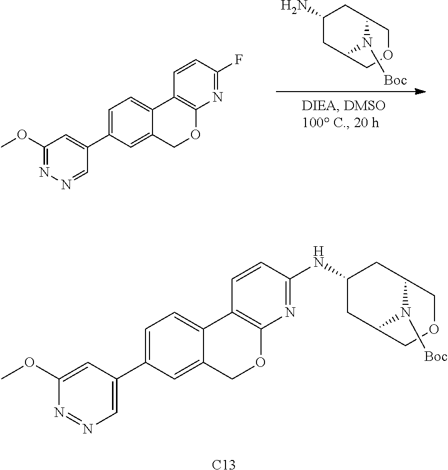 [SMILES: COc1cc(-c2ccc3c(c2)COc2nc(F)ccc2-3)cnn1 ; CC(C)(C)OC(=O)N1[C@@H]2COC[C@H]1C[C@H](N)C2 ; COc1cc(-c2ccc3c(c2)COc2nc(N[C@@H]4C[C@H]5COC[C@@H](C4)N5C(=O)OC(C)(C)C)ccc2-3)cnn1 ; "C13"]

To a stirred mixture of 5-{3-fluoro-6H-isochromeno[3,4-b]pyridin-8-yl}-3-methoxypyridazine (65 mg, 0.210 mmol, 1.00 equiv) and tert-butyl (1R,5S,7r)-7-amino-3-oxa-9-azabicyclo[3.3.1]nonane-9-carboxylate (61.11 mg, 0.252 mmol, 1.2 equiv) in DMSO (1.3 mL, 18.302 mmol, 87.09 equiv) was added DIEA (54.32 mg, 0.420 mmol, 2.0 equiv) at room temperature. The resulting mixture was stirred for 20 h at 100° C. The resulting mixture was cooled to room temperate, diluted with DCM (20 mL), washed with water (5 mL×2) and concentrated under reduced pressure. The residue was purified by Prep-TLC (CH₂Cl₂/MeOH 50:1) to afford tert-butyl (1R,5S,7r)-7-{[8-(6-methoxypyridazin-4-yl)-6H-isochromeno[3,4-b]pyridin-3-yl]amino}-3-oxa-9-azabicyclo[3.3.1]nonane-9-carboxylate (C13, 90 mg, 80%) as a solid that was taken to the next step without further purification. LCMS (ES, m/z): 532[M+H]⁺

### Synthesis of Intermediate C14

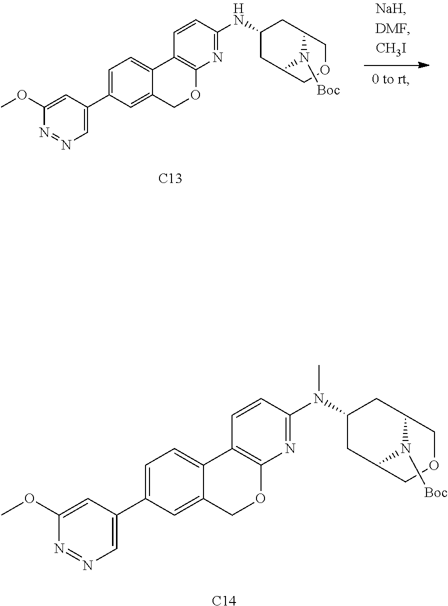 [SMILES: COc1cc(-c2ccc3c(c2)COc2nc(N[C@@H]4C[C@H]5COC[C@@H](C4)N5C(=O)OC(C)(C)C)ccc2-3)cnn1 ; "C13" ; COc1cc(-c2ccc3c(c2)COc2nc(N(C)[C@@H]4C[C@H]5COC[C@@H](C4)N5C(=O)OC(C)(C)C)ccc2-3)cnn1 ; "C14"]

To a solution of tert-butyl (1R,5S,7r)-7-{[8-(6-methoxypyridazin-4-yl)-6H-isochromeno[3,4-b]pyridin-3-yl]amino}-3-oxa-9-azabicyclo[3.3.1]nonane-9-carboxylate (100 mg, 0.188 mmol, 1.00 equiv) in DMF (2 mL, 25.844 mmol, 137.39 equiv) was added sodium hydride (60% in oil, 15.05 mg) at 0° C. The mixture was stirred for 15 min. Methyl iodide (24.03 mg, 0.169 mmol, 0.9 equiv) was added and the mixture was allowed to warm to room temperature and stirred for 2 hr. The reaction mixture was quenched by water and extracted with DCM (10 mL×3). The organic phase was concentrated under reduced pressure. The residue was purified by silica gel column chromatography, eluted with PE/EA (1:1) to afford tert-butyl (1R,5S,7r)-7-{[8-(6-methoxypyridazin-4-yl)-6H-isochromeno[3,4-b]pyridin-3-yl](methyl)amino}-3-oxa-9-azabicyclo[3.3.1]nonane-9-carboxylate (C14, 50 mg, 48%) as a solid that was taken to the next step without further purification. LCMS (ES, m/z): 546[M+H]⁺

### Synthesis of Compound 232

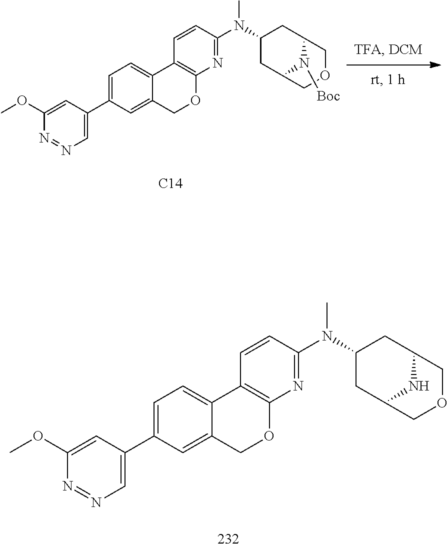 [SMILES: COc1cc(-c2ccc3c(c2)COc2nc(N(C)[C@@H]4C[C@H]5COC[C@@H](C4)N5C(=O)OC(C)(C)C)ccc2-3)cnn1 ; "C14" ; COc1cc(-c2ccc3c(c2)COc2nc(N(C)[C@@H]4C[C@H]5COC[C@@H](C4)N5)ccc2-3)cnn1 ; "232"]

To a solution of tert-butyl (1R,5S,7r)-7-{[8-(6-methoxypyridazin-4-yl)-6H-isochromeno[3,4-b]pyridin-3-yl](methyl)amino}-3-oxa-9-azabicyclo[3.3.1]nonane-9-carboxylate (50 mg, 0.092 mmol, 1.00 equiv) in DCM (0.5 mL) was added TFA (0.5 mL, 6.732 mmol, 73.46 equiv) at room temperature. The resulting mixture was stirred for 1 hr at room temperature and concentrated under reduced pressure. The residue was purified by Prep-HPLC (Condition 7, Gradient 3) to afford (1R,5S,7r)-N-[8-(6-methoxypyridazin-4-yl)-6H-isochromeno[3,4-b]pyridin-3-yl]-N-methyl-3-oxa-9-azabicyclo[3.3.1]nonan-7-amine (232, 11.5 mg, 28%) as a solid. LCMS (ES, m/z): 446[M+H]^{+ 1}H NMR (400 MHz, DMSO-d6) δ 9.34 (d, J=1.9 Hz, 1H), 8.14 (d, J=8.6 Hz, 1H), 7.91 (dd, J=8.2, 2.0 Hz, 1H), 7.86-7.77 (m, 2H), 7.51 (d, J=1.9 Hz, 1H), 6.40 (d, J=8.6 Hz, 1H), 5.30 (s, 2H), 4.09 (s, 3H), 4.02 (d, J=12.4 Hz, 2H), 3.90 (d, J=12.4 Hz, 2H), 3.58 (d, J=4.3 Hz, 2H), 2.87 (s, 3H), 2.21 (t, J=12.0 Hz, 2H), 1.91 (dd, J=13.8, 6.0 Hz, 2H).

### Example 56: Synthesis of Compound 233

### Synthesis of Intermediate C15

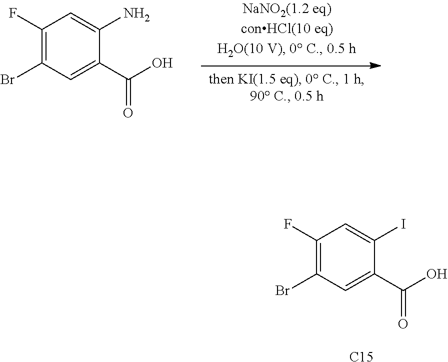 [SMILES: Nc1cc(F)c(Br)cc1C(=O)O ; O=C(O)c1cc(Br)c(F)cc1I ; "C15"]

To a stirred solution of 2-amino-5-bromo-4-fluorobenzoic acid (20.0 g, 85.46 mmol, 1.00 equiv) and NaNO₂ (7.1 g, 102.55 mmol, 1.2 equiv) in H₂O (200 mL), was added conc.HCl (31.1 g, 854.61 mmol, 10 equiv) dropwise at 0° C. under nitrogen atmosphere. The resulting mixture was stirred for 30 min at 0° C. under nitrogen atmosphere. To the above mixture was added KI (21.3 g, 128.19 mmol, 1.5 equiv) in portions over 10 min at 0° C. The resulting mixture was stirred for additional 1 hr at 0° C. Then the resulting mixture was stirred for additional 30 min at 90° C. The resulting mixture was extracted with EtOAc (3×200 mL). The combined organic layers were washed with brine (1×200 mL), dried over anhydrous Na₂SO₄. After filtration, the filtrate was concentrated under reduced pressure. The residue was purified by silica gel column chromatography, eluted with CH₂Cl₂/MeOH (99:1) to afford 5-bromo-4-fluoro-2-iodobenzoic acid (C15, 23.2 g) as a solid that was taken to the next step without further purification. LCMS (ES, m/z): 343 [M+H]⁻

### Synthesis of Intermediate C16

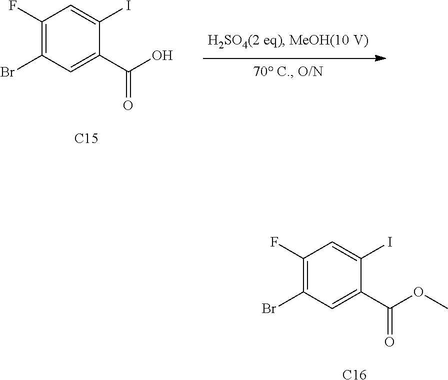

To a stirred solution of 5-bromo-4-fluoro-2-iodobenzoic acid (23.2 g, 67.64 mmol, 1.00 equiv) in MeOH (200 mL) were added H₂SO₄ (13.28 g, 135.27 mmol, 2.00 equiv) dropwise at 70° C. under nitrogen atmosphere. The resulting mixture was stirred for overnight at 70° C. under nitrogen atmosphere. The resulting mixture was diluted with water (200 mL). The mixture was basified to pH 8 with saturated NaHCO₃ (aq.). The resulting mixture was extracted with EtOAc (3×300 mL). The combined organic layers were washed with brine (1×800 mL), dried over anhydrous Na₂SO₄. After filtration, the filtrate was concentrated under reduced pressure. This resulted in methyl 5-bromo-4-fluoro-2-iodobenzoate (C16, 23.2 g) as a solid. LCMS (ES, m/z): 359 [M+H]⁺

### Synthesis of Intermediate C17

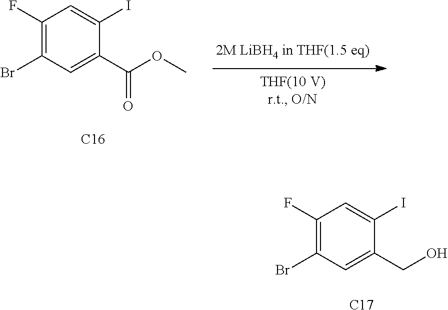

Into a 500-mL 3-necked round-bottom flask purged and maintained with an inert atmosphere of nitrogen, was placed methyl 5-bromo-4-fluoro-2-iodobenzoatE (23.2 g, 64.80 mmol, 1.00 equiv), THE (230 mL). This was the followed by the addition 2M LiBH₄ in THF (1.5 eq) added dropwise with stirring at −60° C. The resulting mixture was allowed to warm and stirred overnight at room temperature. The reaction was then quenched by the addition of 200 mL of water. The resulting solution was extracted with 3×200 mL of ethyl acetate and the organic layers were combined. The resulting mixture was washed with 1×300 ml of saturated aqueous NaCl. The mixture was dried over anhydrous sodium sulfate. The solids were filtered out. The resulting mixture was concentrated under vacuum. The residue was applied onto a silica gel column with ethyl acetate/petroleum ether to afford (5-bromo-4-fluoro-2-iodophenyl)methanol (C17, 15 g, 70%) as a solid that was taken to the next step without further purification. LCMS (ES, m/z): 331 [M+H]⁺

### Synthesis of Intermediate C18

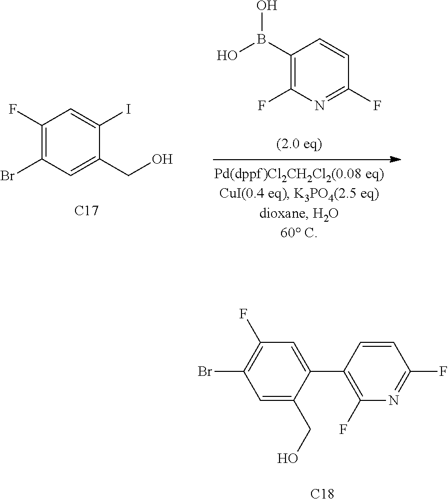 [SMILES: OB(O)c1ccc(F)nc1F ; OCc1cc(Br)c(F)cc1I ; "C17" ; OCc1cc(Br)c(F)cc1-c1ccc(F)nc1F ; "C18"]

Into a 40 mL vial, were added (5-bromo-4-fluoro-2-iodophenyl)methanol (1.0 g, 3.03 mmol, 1.00 equiv), (2,6-difluoropyridin-3-yl)boronic acid (963.63 mg, 6.06 mmol, 2.00 equiv), dioxane (15 mL), K₃po₄ (1.60 g, 7.57 mmol, 2.50 equiv)/H₂O (3 mL) and Pd(dppf)Cl₂·CH₂Cl₂ (197.82 mg, 0.24 mmol, 0.08 equiv), CuI (230.30 mg, 1.21 mmol, 0.40 equiv). Then the reaction mixture was evacuated and flushed three times with nitrogen. The resulting mixture was stirred overnight at 60° C. The reaction was then quenched by the addition of 100 mL of water. The resulting solution was extracted with 3×100 mL of ethyl acetate and the organic layers combined. The resulting mixture was washed with 1×200 ml of saturated aqueous NaCl. The mixture was dried over anhydrous sodium sulfate. The solids were filtered out. The resulting mixture was concentrated under vacuum. The residue was applied onto a silica gel column with ethyl acetate/petroleum ether. This resulted in 2.0 g of (5-bromo-2-(2,6-difluoropyridin-3-yl)-4-fluorophenyl)-methanol as a solid that was taken to the next step without further purification. LCMS (ES, m/z): 318 [M+H]⁺

### Synthesis of Intermediate C19

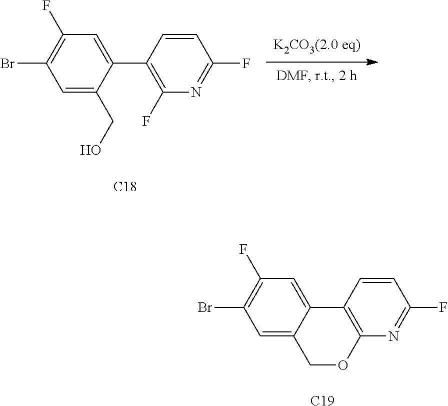 [SMILES: OCc1cc(Br)c(F)cc1-c1ccc(F)nc1F ; "C18" ; Fc1ccc2c(n1)OCc1cc(Br)c(F)cc1-2 ; "C19"]

Into a 8-mL vial, was placed (5-bromo-2-(2,6-difluoropyridin-3-yl)-4-fluorophenyl)methanol (2.0 g, 6.31 mmol, 1.00 equiv), DMF (20 mL), K₂CO₃ (1.74 g, 12.62 mmol, 2.00 equiv). The resulting solution was stirred for 2 hr at room temperature. The reaction mixture was poured into 40 mL of water. The isolated solid was collected. The resulting mixture was concentrated under vacuum. This resulted in 1.2 g (64.0%) of 8-bromo-3,9-difluoro-6H-isochromeno[3,4-b]pyridine as a solid that was taken to the next step without further purification. LCMS (ES, m/z): 298 [M+H]⁺

### Synthesis of Intermediate C20

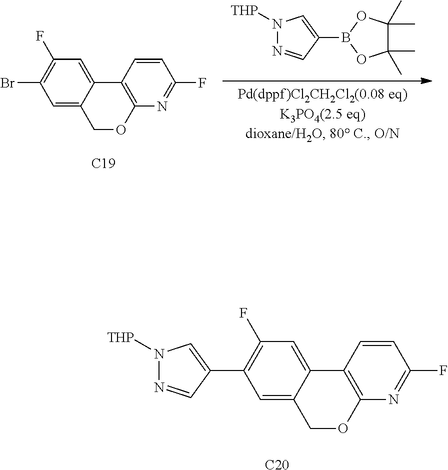

Into a 40 mL vial, were added 8-bromo-3,9-difluoro-6H-isochromeno[3,4-b]pyridine (120 mg, 0.40 mmol, 1.00 equiv), 1-(tetrahydro-2H-pyran-2-yl)-4-(4,4,5,5-tetramethyl-1,3,2-dioxaborolan-2-yl)-1H-pyrazo-le (134.78 mg, 0.48 mmol, 1.2 equiv), dioxane (3 mL), K₃PO₄ (214.14 mg, 1.01 mmol, 2.50 equiv)/H₂O (0.6 mL) and Pd(dppf)Cl₂·CH₂Cl₂ (26.37 mg, 0.032 mmol, 0.08 equiv). Then the reaction mixture was evacuated and flushed three times with nitrogen. The resulting mixture was stirred overnight at 80° C. The reaction was quenched by the addition of 20 mL of water. The resulting solution was extracted with 3×20 mL of ethyl acetate and the organic layers combined. The resulting mixture was washed with 1×50 ml of saturated aqueous NaCl. The mixture was dried over anhydrous sodium sulfate. The solids were filtered out. The resulting mixture was concentrated under vacuum. The residue was applied onto a silica gel column with ethyl acetate/petroleum ether to afford 3,9-difluoro-8-(1-(tetrahydro-2H-pyran-2-yl)-1H-pyrazol-4-yl)-6H-isochromeno[3,4-b]pyridine (C20, 120 mg, 74%) as a solid. LCMS (ES, m/z): 370 [M+H]⁺

### Synthesis of Intermediate C21

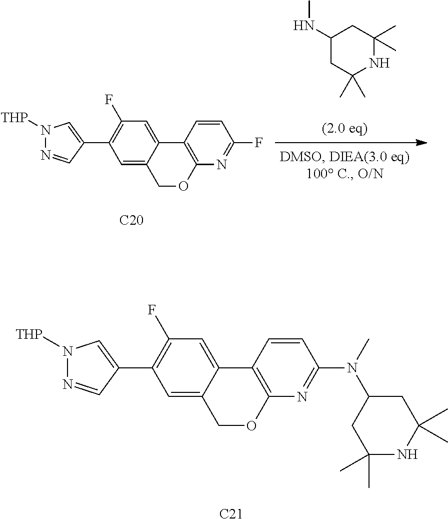

Into a 8-mL vial, was placed 3,9-difluoro-8-(1-(tetrahydro-2H-pyran-2-yl)-1H-pyrazol-4-yl)-6H-isochromeno[3,4-b]pyridine (40.0 mg, 0.108 mmol, 1.00 equiv), DMSO (2 mL), N,2,2,6,6-pentamethyl-piperidin-4-amine (36.85 mg, 0.216 mmol, 2.0 equiv), DIEA (41.9 mg, 0.325 mmol, 3.00 equiv). The resulting solution was stirred overnight at 120° C. The mixture was allowed to cool down to room temperature. The reaction was then quenched by the addition of 10 mL of water. The resulting solution was extracted with 3×10 mL of ethyl acetate and the organic layers were combined. The resulting mixture was washed with 3×20 ml of half saturated aqueous NaCl and 1×20 ml of saturated aqueous NaCl. The mixture was dried over anhydrous sodium sulfate. The solids were filtered out. The resulting mixture was concentrated under vacuum to afford 40 mg (crude) of 9-fluoro-N-methyl-8-(1-(tetrahydro-2H-pyran-2-yl)-1H-pyrazol-4-yl)-N-(2,2,6,6-tetramethylpiperidin-4-yl)-6H-isochromeno[3,4-b]pyridin-3-amine as a solid that was taken to the next step without further purification. LCMS (ES, m/z): 520 [M+H]⁺

### Synthesis of Compound 233

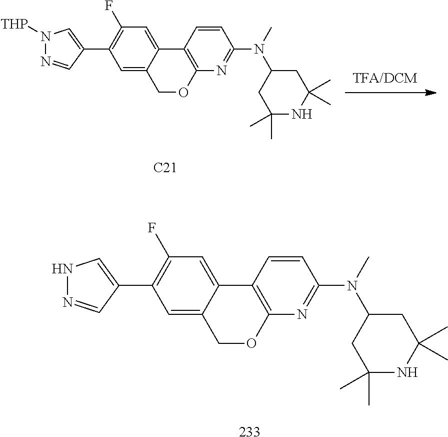

Into a 25-mL round-bottom flask, was 9-fluoro-N-methyl-8-(1-(tetrahydro-2H-pyran-2-yl)-1H-pyrazol-4-yl)-N-(2,2,6,6-tetramethylpiperidin-4-yl)-6H-isochromENo[3,4-b]pyridin-3-amine (40.0 mg), DCM (2 mL), and TFA (0.4 mL). The resulting solution was stirred for 30 min at room temperature. The resulting mixture was concentrated under vacuum. The crude product was purified by Prep-HPLC (Condition 3, Gradient 3) to afford 9-fluoro-N-methyl-8-(1H-pyrazol-4-yl)-N-(2,2,6,6-tetramethylpiperidin-4-yl)-6H-isochromeno[3,4-b]pyridin-3-amine (Compound 233, 10.6 mg, 31%) as a solid.

Compounds 234 and 260 were synthesized according to the general protocol of Compound 233. Characterization data for compounds 233, 234, and 260 are shown in Table 4 below.

TABLE 4
Intermediates and characterization data for
compounds prepared according to Example 56

| Compound | |
|---|---|
| No | Analysis data |
| 233 | LCMS (ES, m/z): 436 [M − HCl] ⁺ |
| | ¹H NMR: (400 MHz, DMSO-d₆): δ 9.23 (d, J = 11.9 Hz, 1H), |
| | 8.19 (d, J = 12.2 Hz, 1H), 8.08 (d, J = 8.6 Hz, 1H), 8.05 (d, J = |
| | 1.9 Hz, 2H), 7.64-7.55 (m, 2H), 6.46 (d, J = 8.6 Hz, 1H), |
| | 5.24 (s, 2H), 5.06 (d, J = 12.9 Hz, 1H), 2.87 (s, 3H), 1.92 |
| | (t, J = 12.9 Hz, 2H), 1.65 (dd, J = 13.2, 3.4 Hz, 2H), 1.49 |
| | (d, J = 14.4 Hz, 12H). |
| 260 | LCMS: (ES, m/z): 393 [M − HCl] ⁺ |
| | ¹H NMR: (400 MHz, DMSO-d₆): δ 9.06 (s, 2H), 8.27 (d, J = |
| | 8.4 Hz, 1H), 8.07 (d, J = 1.9 Hz, 2H), 7.79-7.62 (m, 2H), |
| | 6.56 (d, J = 8.3 Hz, 1H), 5.33 (s, 2H), 5.26 (tt, J = 11.1, 5.9 Hz, |
| | 1H), 4.09 (s, 2H), 2.28 (dt, J = 14.2, 3.5 Hz, 2H), 2.00 (dt, J = |
| | 12.3, 5.8 Hz, 4H), 1.91 (td, J = 10.8, 5.5 Hz, 2H). |
| 234 | LCMS: (ES, m/z): 423 [M − HCl] ⁺ |
| | ¹H NMR: (400 MHz, DMSO-d₆): δ 9.03 (d, J = 12.3 Hz, 1H), |
| | 8.28 (dd, J = 10.4, 4.9 Hz, 2H), 8.07 (d, J = 1.9 Hz, 2H), 7.73 |
| | (d, J = 12.3 Hz, 1H), 7.68 (d, J = 7.7 Hz, 1H), 6.58 (d, J = 8.3 |
| | Hz, 1H), 5.46-5.35 (m, 1H), 5.33 (s, 2H), 2.20 (dd, J = 13.5, |
| | 4.0 Hz, 2H), 1.72 (dd, J = 13.3, 10.5 Hz, 2H), 1.48 (d, J = 4.8 |
| | Hz, 12H). |

### Example 57: Synthesis of Compound 235

### Synthesis of Intermediate C22

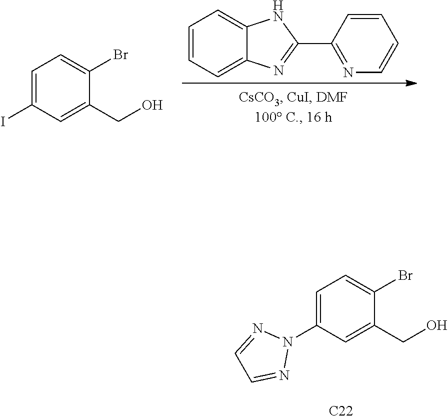 [SMILES: OCc1cc(I)ccc1Br ; c1ccc(-c2nc3ccccc3[nH]2)nc1 ; OCc1cc(-n2nccn2)ccc1Br ; "C22"]

Into a 250 mL 3-necked round-bottom flask were added (2-bromo-5-iodophenyl)methanol (5.00 g, 15.978 mmol, 1.0 equiv), 2-(pyridin-2-yl)-1H-1,3-benzodiazole (0.31 g, 1.598 mmol, 0.1 equiv), Cs₂CO₃ (6.20 g, 31.956 mmol, 2.0 equiv), dimethylformamide (100 mL), copper(I) iodide (0.30 g, 1.598 mmol, 0.1.0 equiv) at 25° C. The resulting mixture was stirred for 16 hr at 100° C. under nitrogen atmosphere. The reaction was quenched with water at 25° C. The aqueous layer was extracted with ethyl acetate (2×200 mL). The combined organic layer was concentrated under reduced pressure. The residue was purified by silica gel column chromatography, eluted with PE/EA (1:1) to afford [2-bromo-5-(1,2,3-triazol-2-yl)phenyl]methanol (C22, 1.3 g, 28%) as a solid that was taken to the next step without further purification. LCMS (ES, m/z): 254 [M+H]⁺

### Synthesis of Intermediate C23

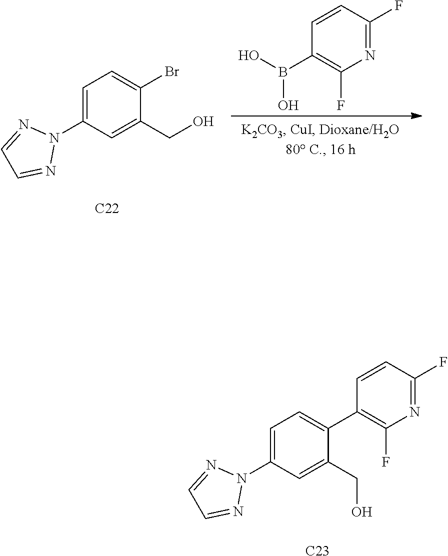 [SMILES: OCc1cc(-n2nccn2)ccc1Br ; OB(O)c1ccc(F)nc1F ; "C22" ; OCc1cc(-n2nccn2)ccc1-c1ccc(F)nc1F ; "C23"]

Into a 100 mL 3-necked round-bottom flask were added [2-bromo-5-(1,2,3-triazol-2-yl)phenyl]methanol (1.40 g, 5.510 mmol, 1.0 equiv), 2,6-difluoropyridin-3-ylboronic acid (1.05 g, 6.612 mmol, 1.2 equiv), copper(I) iodide (0.10 g, 0.551 MMol, 0.1 equiv), water (5 mL), Dioxane (20 mL) and Pd(dppf)Cl₂ (0.40 g, 0.551 mmol, 0.1 equiv) at 25 degrees C. The resulting mixture was stirred for 16 h at 80 degrees C. under nitrogen atmosphere. The resulting mixture was concentrated under reduced pressure. The residue was purified by silica gel column chromatography, eluted with PE/EA (2:1) to afford [2-(2,6-difluoropyridin-3-yl)-5-(1,2,3-triazol-2-yl)phenyl]methanol (C23, 1.2 g, 68%) as an oil that was taken to the next step without further purification. LCMS (ES, m/z): 289 [M+H]⁺

### Synthesis of Intermediate C24

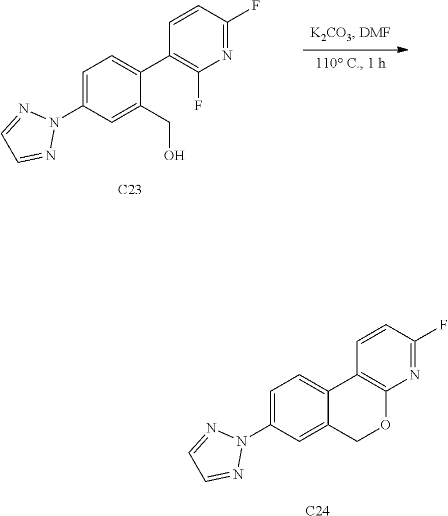 [SMILES: OCc1cc(-n2nccn2)ccc1-c1ccc(F)nc1F ; "C23" ; Fc1ccc2c(n1)OCc1cc(-n3nccn3)ccc1-2 ; "C24"]

Into a 100 mL 3-necked round-bottom flask were added [2-(2,6-difluoropyridin-3-yl)-5-(1,2,3-triazol-2-yl)phenyl]methanol (1.5 g, 5.204 mmol, 1.0 equiv) and K₂CO₃ (2.17 g, 15.612 mmol, 3.0 equiv) at 25° C. The resulting mixture was stirred for 1 hr at 110° C. under nitrogen atmosphere. The reaction was diluted with water at 25° C. The aqueous layer was extracted with EtOAc (2×100 mL). The combined organic layer was concentrated under reduced pressure. The residue was purified by silica gel column chromatography, eluted with PE/EA (1:5) to afford 2-{3-fluoro-6H-isochromeno[3,4-b]pyridin-8-yl}-1,2,3-triazole (C24, 1.3 g, 83%) as a solid that was taken to the next step without further purification. LCMS (ES, m/z): 269 [M+H]⁺

### Synthesis of Intermediate C25

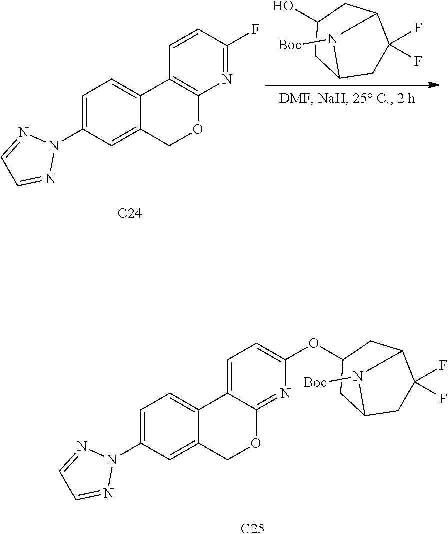

Into a 40 mL round-bottom flask were added 2-{3-fluoro-6H-isochromeno[3,4-b]pyridin-8-yl}-1,2,3-triazole (60 mg, 0.224 mmol, 1.0 equiv), tert-butyl 6,6-difluoro-3-hydroxy-8-azabicyclo[3.2.1]octane-8-carboxylate (70.6 mg, 0.269 mmol, 1.2 equiv) and sodium hydride (10.7 mg, 0.448 mmol, 2.0 equiv) at 0° C. The resulting mixture was stirred for 1 hr at 25° C. under nitrogen atmosphere. The reaction was quenched with water at 0° C. The aqueous layer was extracted with EtOAc (2×50 mL). The resulting mixture was concentrated under reduced pressure to afford tert-butyl 6,6-difluoro-3-{[8-(1,2,3-triazol-2-yl)-6H-isochromeno[3,4-b]pyridin-3-yl]oxy}-8-azabicyclo[3.2.1]octane-8-carboxylate (C25, 120 mg, 94%) as a solid that was taken to the next step without further purification. LCMS (ES, m/z): 512 [M+H]⁺

### Synthesis of Compound 235

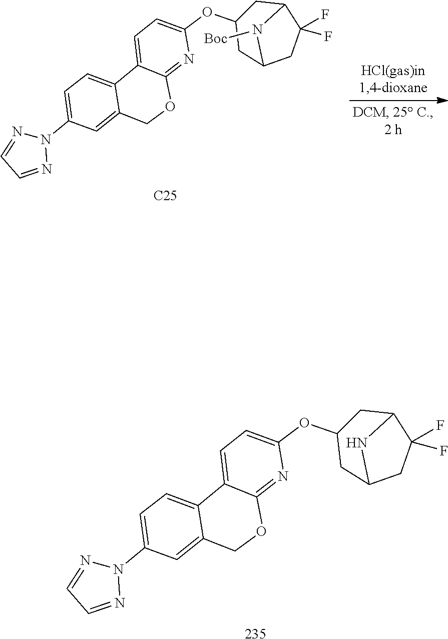 [SMILES: CC(C)(C)OC(=O)N1C2CC(Oc3ccc4c(n3)OCc3cc(-n5nccn5)ccc3-4)CC1C(F)(F)C2 ; "C25" ; FC1(F)CC2CC(Oc3ccc4c(n3)OCc3cc(-n5nccn5)ccc3-4)CC1N2 ; "235"]

Into a 40 mL round-bottom flask were added tert-butyl 6,6-difluoro-3-{[8-(1,2,3-triazol-2-yl)-6H-isochromeno[3,4-b]pyridin-3-yl]oxy}-8-azabicyclo[3.2.1]octane-8-carboxylate (110 mg, 0.215 mmol, 1.0 equiv) and HCl (gas) in 1,4-dioxane (1 mL) at 25° C. The resulting mixture was stirred for additional 2 hr at 25° C. The resulting mixture was concentrated under reduced pressure. The crude product (mg) was purified by Prep-HPLC (Condition 6, Gradient 3) to afford 6,6-difluoro-3-{[8-(1,2,3-triazol-2-yl)-6H-isochromeno[3,4-b]pyridin-3-yl]oxy}-8-azabicyclo[3.2.1]octane (Compound 235, 60.3 mg, 64%) as a solid. LCMS (ES, m/z): 412 [M+H]+¹H NMR (300 MHz, Chloroform-d) δ 9.62-9.59 (m, 2H), 8.32 (d, J=8.4 Hz, 1H), 8.16 (s, 2H), 8.08-7.92 (m, 3H), 6.61 (d, J=8.3 Hz, 1H), 5.46 (s, 2H), 5.38 (dd, J=11.5, 5.7 Hz, 1H), 4.42-4.27 (m, 2H), 2.81-2.57 (m, 3H), 2.42 (d, J=13.0 Hz, 1H), 2.06 (t, J=12.3 Hz, 1H), 1.92 (t, J=12.4 Hz, 1H).

### Example 58: Synthesis of Compound 238

### Synthesis of Intermediate C26

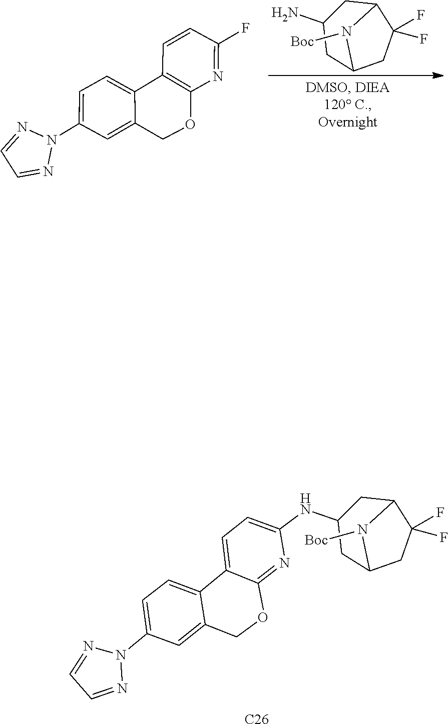 [SMILES: Fc1ccc2c(n1)OCc1cc(-n3nccn3)ccc1-2 ; CC(C)(C)OC(=O)N1C2CC(N)CC1C(F)(F)C2 ; CC(C)(C)OC(=O)N1C2CC(Nc3ccc4c(n3)OCc3cc(-n5nccn5)ccc3-4)CC1C(F)(F)C2 ; "C26"]

Into a 40 mL round-bottom flask were added 2-{3-fluoro-6H-isochromeno[3,4-b]pyridin-8-yl}-1,2,3-triazole (270.0 mg, 1.007 mmol, 1.0 equiv), tert-butyl 3-amino-6,6-difluoro-8-azabicyclo[3.2.1] octane-8-carboxylate (396.0 mg, 1.510 mmol, 1.5 equiv), DMSO (3 mL), and DIEA (1300.8 mg, 10.070 mmol, 10.0 equiv) at 25° C. The resulting mixture was stirred overnight at 120° C. The reaction was quenched with water at 25° C. The aqueous layer was extracted with EtOAc (2×50 mL). The combined organic layers were washed with water (1×100 mL), brine (1×100 mL), dried over anhydrous Na₂SO₄. After filtration, the filtrate was concentrated under reduced pressure. The residue was purified by silica gel column chromatography, eluted with PE/EA (1:1) to afford tert-butyl 6,6-difluoro-3-{[8-(1,2,3-triazol-2-yl)-6H-isochromeno[3,4-b]pyridin-3-yl]amino}-8-azabicyclo[3.2.1]octane-8-carboxylate (C26, 270 mg, 47%) as a solid that was taken to the next step without further purification. LCMS (ES, m/z): 511 [M+H]⁺

### Synthesis of Compound 238

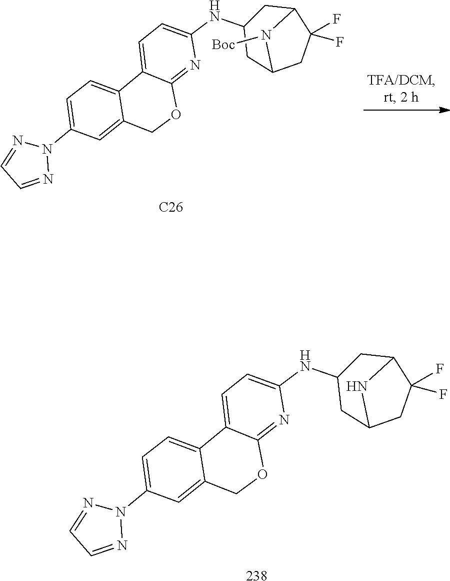 [SMILES: CC(C)(C)OC(=O)N1C2CC(Nc3ccc4c(n3)OCc3cc(-n5nccn5)ccc3-4)CC1C(F)(F)C2 ; "C26" ; FC1(F)CC2CC(Nc3ccc4c(n3)OCc3cc(-n5nccn5)ccc3-4)CC1N2 ; "238"]

Into a 8 mL round-bottom flask were added tert-butyl 6,6-difluoro-3-{[8-(1,2,3-triazol-2-yl)-6H-isochromeno[3,4-b]pyridin-3-yl]amino}-8-azabicyclo[3.2.1]octane-8-carboxylaTE (50.0 mg, 0.098 Mmol, 1.0 equiv), DCM (1 mL) and TFA (0.3 mL) at 25° C. The resulting mixture was stirred for 2 hr at room temperature. The resulting mixture was concentrated under reduced pressure. The crude product was purified by Prep-HPLC (condition 5, Gradient 4) to afford 6,6-difluoro-N-[8-(1,2,3-triazol-2-yl)-6H-isochromeno[3,4-b]pyridin-3-yl]-8-azabicyclo[3.2.1]octan-3-amine (Compound 238, 10 mg, 24%) as a solid. LCMS (ES, m/z): 411 [M+H]^{+ 1}H NMR (300 MHz, Chloroform-d) δ 8.11-8.10 (m, 2H), 7.99-7.86 (m, 3H), 7.73 (d, J=8.5 Hz, 1H), 6.86 (d, J=7.9 Hz, 1H), 6.23 (d, J=8.4 Hz, 1H), 5.30 (s, 2H), 4.22-4.21 (m, 1H), 3.56-3.55 (m, 1H), 3.37-3.36 (m, 1H), 2.83-2.82 (m, 1H), 2.36-2.35 (m, 1H), 2.24-2.10 (m, 1H), 2.10-2.08 (m, 1H), 1.95-1.93 (m, 1H), 1.58-1.34 (m, 2H).

### Example 59: Synthesis of Compound 239

### Synthesis of Intermediate C27

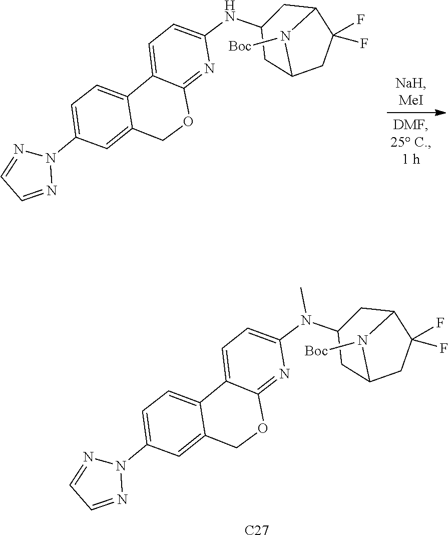 [SMILES: CC(C)(C)OC(=O)N1C2CC(Nc3ccc4c(n3)OCc3cc(-n5nccn5)ccc3-4)CC1C(F)(F)C2 ; CN(c1ccc2c(n1)OCc1cc(-n3nccn3)ccc1-2)C1CC2CC(F)(F)C(C1)N2C(=O)OC(C)(C)C ; "C27"]

Into a 40 mL round-bottom flask were added tert-butyl 6,6-difluoro-3-{[8-(1,2,3-triazol-2-yl)-6H-isochromeno[3,4-b]pyridin-3-yl]amino}-8-azabicyclo[3.2.1]octane-8-carboxylatE (100.0 mg, 0.196 mmol, 1.0 equiv), DMF (2 mL), NaH (9.4 mg, 0.392 mmol, 2.0 equiv) and Mel (55.6 mg, 0.392 mmol, 2.0 equiv) at 0° C. The resulting mixture was stirred for 1 hr at 25° C. The reaction was quenched with ice water at 0° C. The aqueous layer was extracted with EtOAc (2×50 mL). The combined organic layers were dried by Na₂SO₄ and concentrated under reduced pressure. This resulted in tert-butyl 6,6-difluoro-3-{methyl[8-(1,2,3-triazol-2-yl)-6H-isochromeno[3,4-b]pyridin-3-yl]amino}-8-azabicyclo[3.2.1]octane-8-carboxylate (C27, 95 mg, 87%) as an oil that was taken to the next step without further purification. LCMS (ES, m/z): 525 [M+H]⁺

### Synthesis of Compound 239

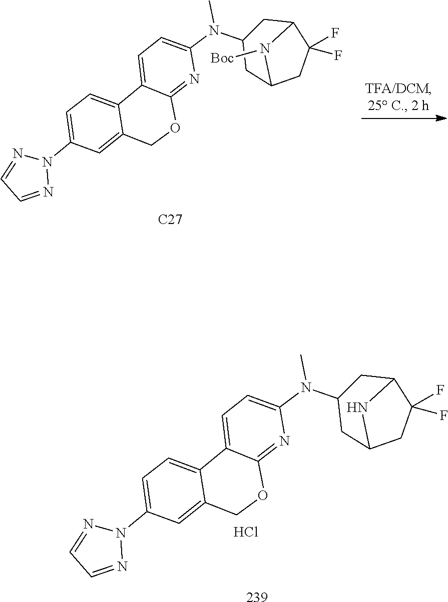 [SMILES: CN(c1ccc2c(n1)OCc1cc(-n3nccn3)ccc1-2)C1CC2CC(F)(F)C(C1)N2C(=O)OC(C)(C)C ; "C27" ; CN(c1ccc2c(n1)OCc1cc(-n3nccn3)ccc1-2)C1CC2CC(F)(F)C(C1)N2 ; Cl ; "239"]

Into a 8 mL round-bottom flask were added tert-butyl 6,6-difluoro-3-{methyl[8-(1,2,3-triazol-2-yl)-6H-isochromeno[3,4-b]pyridin-3-yl]amino}-8-azabicyclo[3.2.1]octane-8-carboxylatE (100.0 mg, 0.191 mmol, 1.0 equiv), DCM (1 mL) and TFA (0.3 mL) at 25 degrees C. The resulting mixture was stirred for 2 hr at 25° C. The resulting mixture was concentrated under reduced pressure. The crude product was purified by Prep-HPLC (Condition 6, Gradient 3) to afford 6,6-difluoro-N-methyl-N-[8-(1,2,3-triazol-2-yl)-6H-isochromeno[3,4-b]pyridin-3-yl]-8-azabicyclo[3.2.1]octan-3-amine (Compound 239, 10 mg, 11%) as a solid. LCMS (ES, m/z): 425 [M+H]+¹H NMR (300 MHz, Chloroform-d) δ 10.80-10.79 (m, 1H), 9.92-9.91 (m, 1H), 8.11-8.10 (m, 3H), 8.01-7.88 (m, 2H), 7.82 (d, J=8.5 Hz, 1H), 6.43 (d, J=8.6 Hz, 1H), 5.34 (s, 2H), 5.19 (dd, J=12.7, 6.9 Hz, 1H), 4.41-4.27 (m, 2H), 2.97 (s, 3H), 2.77-2.62 (m, 2H), 2.53-2.52 (m, 1H), 2.38 (dd, J=27.5, 14.3 Hz, 1H), 1.99-1.79 (m, 2H).

### Example 60: Synthesis of Compound 240

### Synthesis of Intermediate C28

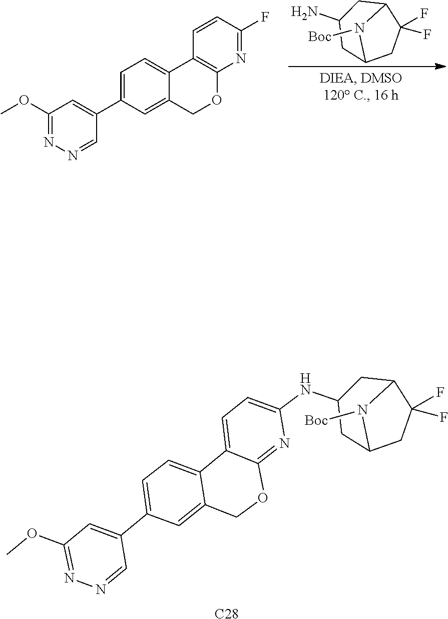 [SMILES: COc1cc(-c2ccc3c(c2)COc2nc(F)ccc2-3)cnn1 ; CC(C)(C)OC(=O)N1C2CC(N)CC1C(F)(F)C2 ; COc1cc(-c2ccc3c(c2)COc2nc(NC4CC5CC(F)(F)C(C4)N5C(=O)OC(C)(C)C)ccc2-3)cnn1 ; "C28"]

To a stirred mixture of 5-{3-fluoro-6H-isochromeno[3,4-b]pyridin-8-yl}-3-methoxypyridazine (40.0 mg, 0.129 mmol, 1.0 equiv) and tert-butyl 3-amino-6,6-difluoro-8-azabicyclo[3.2.1]octane-8-carboxylate (33.9 mg, 0.129 mmol, 1.0 equiv) in DMSO (0.4 mL) was added DIEA (50.1 mg, 0.387 mmol, 3.0 equiv) at room temperature. The resulting mixture was stirred for 16 hr at 120° C. The resulting mixture was diluted with water (1 mL) and extracted with EA (3×2 mL). The combined organic layers were washed with water (3×3 mL), dried over anhydrous Na₂SO₄. After filtration, the filtrate was concentrated under reduced pressure to afford tert-butyl 6,6-difluoro-3-{[8-(6-methoxypyridazin-4-yl)-6H-isochromeno[3,4-b]pyridin-3-yl]amino}-8-azabicyclo[3.2.1]octane-8-carboxylate (C28, 70 mg, 49%) as a solid that was taken to the next step without further purification. LCMS (ES, m/z): 552 [M+H]⁺

### Synthesis of Compound 240

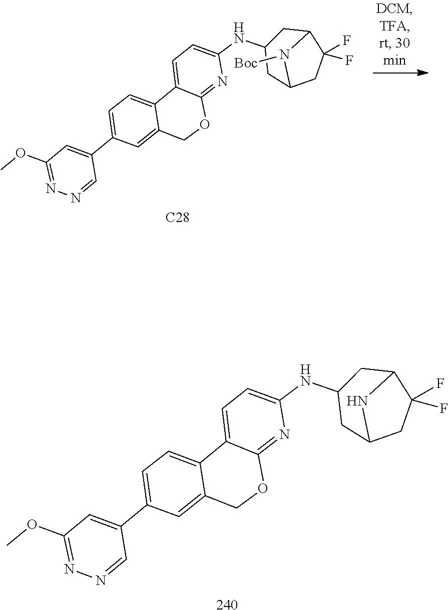 [SMILES: COc1cc(-c2ccc3c(c2)COc2nc(NC4CC5CC(F)(F)C(C4)N5C(=O)OC(C)(C)C)ccc2-3)cnn1 ; "C28" ; COc1cc(-c2ccc3c(c2)COc2nc(NC4CC5CC(F)(F)C(C4)N5)ccc2-3)cnn1 ; "240"]

To a stirred mixture of tert-butyl 6,6-difluoro-3-{[8-(6-methoxypyridazin-4-yl)-6H-isochromeno[3,4-b]pyridin-3-yl]amino}-8-azabicyclo[3.2.1]octane-8-carboxylate (63.0 mg, 0.114 mmol, 1.0 equiv) in DCM (1.8 mL) was added TFA (0.6 mL) dropwise at room temperature. The resulting mixture was stirred for 30 min at room temperature. The resulting mixture was concentrated under reduced pressure. The crude product was purified by Prep-HPLC (Condition 5, Gradient 4) to afford 6,6-difluoro-N-[8-(6-methoxypyridazin-4-yl)-6H-isochromeno[3,4-b]pyridin-3-yl]-8-azabicyclo[3.2.1]octan-3-amine (Compound 240, 5.8 mg, 11%) as a solid. LCMS (ES, m/z): 452 [M+H]^{+ 1}H NMR (400 MHz, DMSO-d₆) δ 9.33 (d, J=1.9 Hz, 1H), 7.96 (d, J=8.5 Hz, 1H), 7.88 (dd, J=8.2, 2.0 Hz, 1H), 7.81 (d, J=2.0 Hz, 1H), 7.72 (d, J=8.2 Hz, 1H), 7.50 (d, J=2.0 Hz, 1H), 6.92 (d, J=8.0 Hz, 1H), 6.24 (d, J=8.4 Hz, 1H), 5.26 (s, 2H), 4.23-4.22 (m, 1H), 4.08 (s, 3H), 3.55-3.54 (m, 1H), 2.84-2.83 (m, 1H), 2.49-2.32 (m, 1H), 2.20-2.13 (m, 1H), 2.10-2.07 (m, 1H), 1.95-1.92 (m, 1H), 1.52 (t, J=11.7 Hz, 1H), 1.41 (t, J=12.4 Hz, 1H).

### Example 61: Synthesis of Compound 241

### Synthesis of Intermediate C29

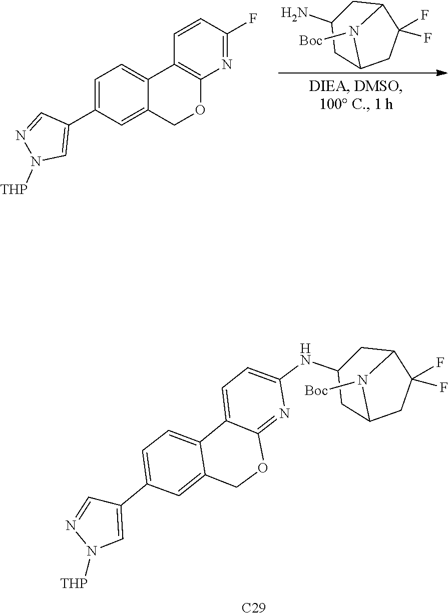

Into a 8 mL vial were added 4-{3-fluoro-6H-isochromeno[3,4-b]pyridin-8-yl}-1-(oxan-2-yl)pyrazole (100 mg, 0.285 mmol, 1.00 equiv), tert-butyl 3-amino-6,6-difluoro-8-azabicyclo[3.2.1]octane-8-carboxylate (111.9 mg, 0.427 mmol, 1.5 equiv), DIEA (183.9 mg, 1.425 mmol, 5.0 equiv) and DMSO (1 mL) at room temperature. The resulting mixture was stirred for 16 h at 120° C. The Resulting mixture was diluted with water (3 mL). The resulting mixture was extracted with EtOAc (3×4 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 6,6-difluoro-3-({8-[1-(oxan-2-yl)pyrazol-4-yl]-6H-isochromeno[3,4-b]pyridin-3-yl}amino)-8-azabicyclo[3.2.1]octane-8-carboxylate (C29, 85 mg, 50%) as an oil that was taken to the next step without further purification. LCMS (ES, m/z): 594 [M+H]⁺

### Synthesis of Compound 241

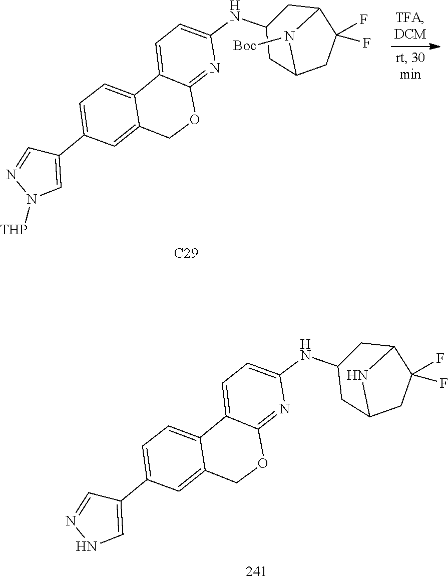

Into a 8 mL Vial were added trifluoroacetaldehyde (0.1 mL), DCM (0.2 mL) and tert-butyl 6,6-difluoro-3-({8-[1-(oxan-2-yl)pyrazol-4-yl]-6H-isochromeno[3,4-b]pyridin-3-yl}amino)-8-azabicyclo[3.2.1]octane-8-carboxylate (35 mg, 0.059 mmol, 1.0 equiv) at room temperature. The resulting mixture was stirred for 30 min at room temperature. The resulting mixture was concentrated under reduced pressure. The residue was purified by Prep-HPLC with the following conditions (Condition 6, Gradient 4) to afford 6,6-difluoro-N-[8-(1H-pyrazol-4-yl)-6H-isochromeno[3,4-b]pyridin-3-yl]-8-azabicyclo[3.2.1]octan-3-amine (Compound 241, 5.6 mg, 23%) as a solid. LCMS (ES, m/z): 410 [M+H]^{+ 1}H NMR (400 MHz, DMSO-d₆) δ 9.61 (m, 1H), 9.40 (m, 1H), 8.10-8.05 (m, 2H), 7.92 (d, J=8.4 Hz, 1H), 7.57 (d, J=2.3 Hz, 2H), 7.45 (s, 1H), 6.95 (d, J=7.2 Hz, 1H), 6.28 (d, J=8.4 Hz, 1H), 5.21 (s, 2H), 4.33-4.27 (m, 3H), 2.73-2.70 (m, 2H), 2.50-2.40 (m, 1H), 2.22 (d, J=13.9 Hz, 1H), 1.87 (t, J=12.8 Hz, 1H), 1.72 (t, J=13.2 Hz, 1H).

### Example 62: Synthesis of Compound 242

### Synthesis of Intermediate C30

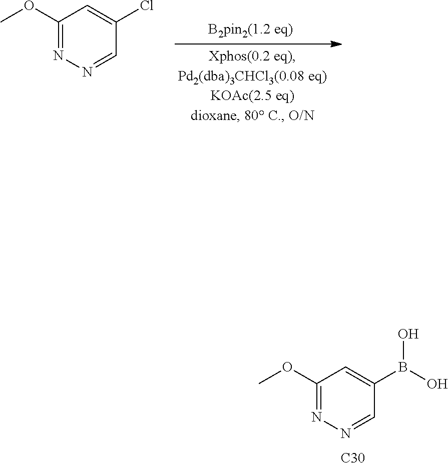

Into a 30-mL vial purged, was placed 5-chloro-3-methoxypyridazine (400 mg, 2.77 mmol, 1.00 equiv), 4,4,5,5-tetramethyl-2-(tetramethyl-1,3,2-dioxaborolan-2-yl)-1,3,2-dioxaborolane (846.66 mg, 3.33 mmol, 1.20 equiv), KOAc (680.55 mg, 6.94 mmol, 2.50 equiv), XPhos (264.44 mg, 0.55 mmol, 0.20 equiv), Pd₂(dba)₃ CHCl₃ (229.99Mg, 0.22 mmol, 0.08 equiv) and dioxane (12 mL). Then the reaction mixture was evacuated and flushed three times with nitrogen. The resulting mixture was stirred overnight at 80° C. The solids were filtered out. The product was added to the next step directly without further purification. LCMS (ES, m/z): 155 [M+H]⁺

### Synthesis of Intermediate C31

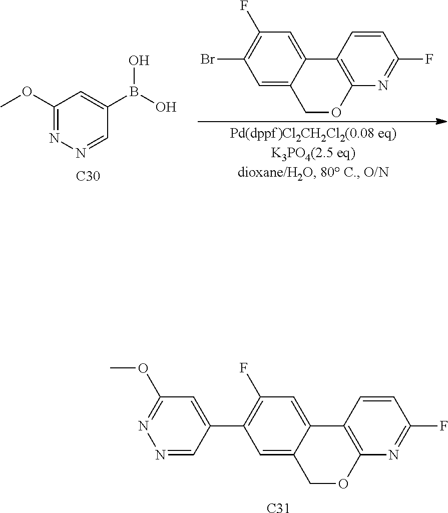 [SMILES: COc1cc(B(O)O)cnn1 ; Fc1ccc2c(n1)OCc1cc(Br)c(F)cc1-2 ; "C30" ; COc1cc(-c2cc3c(cc2F)-c2ccc(F)nc2OC3)cnn1 ; "C31"]

Into a 40 mL vial, were added 8-bromo-3,9-difluoro-6H-isochromeno[3,4-b]pyridine (400 mg, 1.34 mmol, 1.00 equiv), (6-methoxypyridazin-4-yl)boronic acid (300.07 mg, 1.95 mmol, 1.45 equiv), dioxane (8 mL), K₃PO₄ (713.8 mg, 3.36 mmol, 2.50 equiv)/H₂O (1.6 mL) and Pd(dppf)Cl₂·CH₂Cl₂ (87.92 mg, 0.107 mmol, 0.08 equiv). Then the reaction mixture was evacuated and flushed three times with nitrogen. The resulting mixture was stirred overnight at 80° c. The reaction mixture was poured into 40 mL of water. The isolated solid was collected. The resulting mixture was concentrated under vacuum to afford 3,9-difluoro-8-(6-methoxypyridazin-4-yl)-6H-isochromeno[3,4-b]pyridine (C31, 340 mg) as a solid that was taken to the next step without further purification. LCMS (ES, m/z): 328 [M+H]⁺

### Synthesis of Intermediate C32

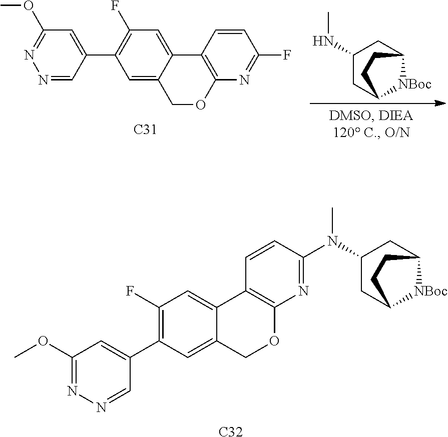

Into a 8-mL vial, was placed 3,9-difluoro-8-(6-methoxypyridazin-4-yl)-6H-iSOchromeno[3,4-b]pyridine (110 mg), DMSO (2 mL), tert-butyl (1R,3s,5S)-3-(methylamino)-8-azabicyclo[3.2.1]octane-8-carboxylate (161.46 mg, 0.67 mmol, 2.0 equiv), DIEA (130.18 mg, 1.01 mmol, 3.00 equiv). The resulting solution was stirred overnight at 120° C. The mixture was allowed to cool down to room temperature. The reaction was then quenched by the addition of 10 mL of water. The resulting solution was extracted with 3×10 mL of ethyl acetate and the organic layers combined. The resulting mixture was washed with 3×20 mL of half saturated aqueous NaCl and 1×20 mL of saturated aqueous NaCl. The mixture was dried over anhydrous sodium sulfate. The solids were filtered out. The resulting mixture was concentrated under vacuum. The crude product was purified by Prep-HPLC with the following conditions: Mobile Phase A: Water (10 mmol/L NH₄HCO₃), Mobile Phase B: ACN. This afforded tert-butyl (1R,3s,5S)-3-((9-fluoro-8-(6-methoxypyridazin-4-yl)-6H-isochromeno[3,4-b]pyridin-3-yl)(methyl)amino)-8-azabicyclo[3.2.1]octane-8-carboxylate (C32, 50 mg, 27%) as a solid that was taken to the next step without further purification. LCMS (ES, m/z): 548 [M+H]⁺

### Synthesis of Compound 242

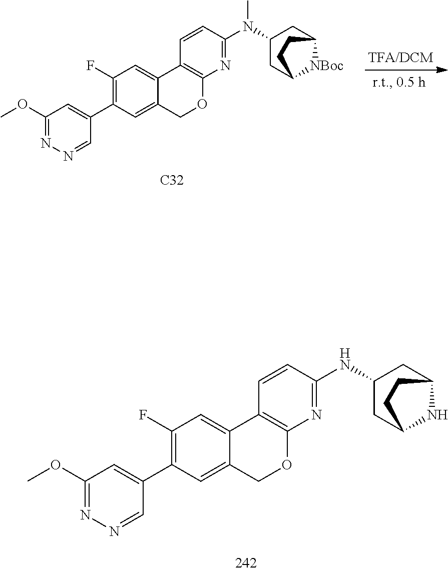

Into a 25-mL round-bottom flask, was tert-butyl (1R,3s,5S)-3-((9-fluoro-8-(6-methoxypyridazin-4-yl)-6H-isochromeno[3,4-b]pyridin-3-yl)(methyl)amino)-8-azabicyclo[3.2.1]octane-8-carboXYlate (50.0 mg, 0.091 mmol, 1.00 equiv), DCM (2 mL), TFA (0.4 mL). The resulting solution was stirred for 30 min at room temperature. The resulting mixture was concentrated under vacuum. The crude product was purified by Prep-HPLC (Condition 2, Gradient 13). This afforded of N-((1R,3s,5S)-8-azabicyclo[3.2.1]octan-3-yl)-9-fluoro-8-(6-methoxypyridazin-4-yl)-6H-isochromeno [3,4-b]pyridin-3-amine (Compound 242, 24.1 mg, 58.9%) as a solid.

Compounds 237, 243, and 244 were synthesized according to the general protocol of Compound 242. Characterization data for compounds 237 and 242-244 and are shown in Table 5 below.

TABLE 5
Characterization data for compounds prepared
according to the protocol described in Example 62.

| Compound | |
|---|---|
| No | Analysis data |
| 242 | LCMS (ES, m/z): 448 [M + H] ⁺ |
| | ¹HNMR (400 MHz, DMSO-d₆): δ 9.15 (t, J = 1.8 Hz, |
| | 1H), 8.09 (d, J = 8.7 Hz, 1H), 7.74-7.63 (m, 2H), 7.40 |
| | (dd, J = 1.9, 0.9 Hz, 1H), 6.40 (d, J = 8.7 Hz, 1H), 5.27 |
| | (s, 2H), 4.88 (s, 1H), 4.09 (s, 3H), 3.49 (s, 2H), 2.84 (s, |
| | 3H), 1.71 (dt, J = 11.2, 8.6 Hz, 6H), 1.49-1.41 (m, |
| | 2H). |
| 243 | LCMS: (ES, m/z): 435 [M + H] ⁺ |
| | ¹H NMR: (400 MHz, DMSO-d₆): δ 9.16 (t, J = 1.8 Hz, |
| | 1H), 8.31 (d, J = 8.5 Hz, 1H), 7.86 (d, J = 12.2 Hz, 1H), |
| | 7.72 (d, J = 7.7 Hz, 1H), 7.43 (dd, J = 1.9, 1.0 Hz, 1H), |
| | 6.54 (d, J = 8.3 Hz, 1H), 5.37 (s, 2H), 5.22 (tt, J = 11.2, |
| | 6.0 Hz, 1H), 4.10 (s, 3H), 3.49 (d, J = 4.2 Hz, 2H), 2.04 |
| | (ddd, J = 11.8, 5.7, 2.7 Hz, 2H), 1.76-1.59 (m, 4H), |
| | 1.59-1.45 (m, 2H). |
| 244 | LCMS: (ES, m/z): 465 [M + H] ⁺ |
| | ¹H NMR: (400 MHz, DMSO-d₆): δ 9.15 (t, J = 1.8 Hz, |
| | 1H), 8.30 (d, J = 8.5 Hz, 1H), 7.83 (d, J = 12.1 Hz, 1H), |
| | 7.70 (d, J = 7.7 Hz, 1H), 7.42 (t, J = 1.4 Hz, 1H), 6.54 |
| | (d, J = 8.3 Hz, 1H), 5.36 (s, 3H), 4.08 (s, 3H), 1.94 (dd, |
| | J = 12.0, 4.1 Hz, 2H), 1.19 (s, 8H), 1.09 (s, 6H). |
| 237 | LCMS: (ES, m/z): 478 [M + H] ⁺ |
| | ¹H NMR: (400 MHz, DMSO-d₆): δ 9.15 (t, J = 1.8 Hz, |
| | 1H), 8.11 (d, J = 8.8 Hz, 1H), 7.78-7.59 (m, 2H), 7.40 |
| | (dd, J = 1.9, 0.9 Hz, 1H), 6.42 (d, J = 8.7 Hz, 1H), 5.27 |
| | (s, 2H), 4.92 (s, 1H), 4.09 (s, 3H), 2.86 (s, 3H), 1.49- |
| | 1.35 (m, 4H), 1.24 (s, 6H), 1.09 (s, 6H). |

### Example 63: Synthesis of Compound 245

### Synthesis of Intermediate C33

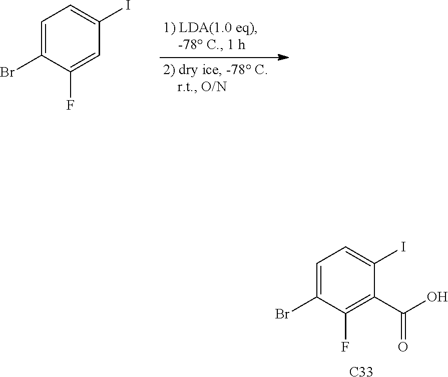 [SMILES: Fc1cc(I)ccc1Br ; O=C(O)c1c(I)ccc(Br)c1F ; "C33"]

To a stirred solution of 1-bromo-2-fluoro-4-iodobeNZene (3.0 g, 1.00 mMOl, 1.00 equiv) in THE (30 mL) was added LDA (5 mL, 1.00 mmol, 1.00 equiv) dropwise at −78° C. under nitrogen atmosphere. The resulting solution was stirred for 1 hr and then dry ice was added portion wise at −78° C. The resulting mixture was allowed to warm and stirred overnight at room temperature. The resulting mixture was quenched with 1N HCl and extracted with 5% MeOH in DCM (3×50 mL). The resulting mixture was washed with 1×100 ml of saturated aqueous NaCl. The mixture was dried over anhydrous sodium sulfate. The solids were filtered out. This afforded 3-bromo-2-fluoro-6-iodobenzoic acid (C33, 3.1 g) as a solid. LCMS (ES, m/z): 343 [M−H]⁻

### Synthesis of Intermediate C34

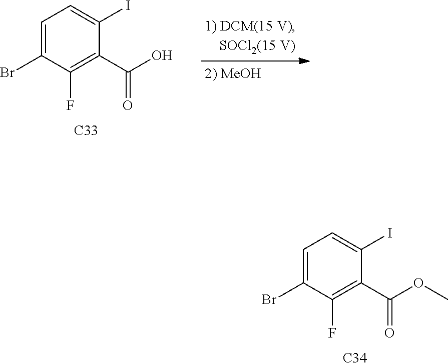

To a stirred solution of 3-bromo-2-fluoro-6-iodobenzoic acid (3.1 g) in DCM (45 mL) were added SOCl₂ (45 mL) dropwise at 0° C. under nitrogen atmosphere. The resulting mixture was stirred overnight at room temperature. The resulting mixture was concentrated and methanol (45 mL) was added. Then the mixture was stirred for 1 hr at room temperature. The resulting mixture was evaporated and quenched with saturated sodium bicarbonate at 0° C. and extracted with 3×100 mL of ethyl acetate and the organic layers were combined. The resulting mixture was washed with 1×200 ml of saturated aqueous NaCl. The mixture was dried over anhydrous sodium sulfate. The solids were filtered out. The resulting mixture was concentrated under vacuum. This resulted in 2.4 g of methyl 3-bromo-2-fluoro-6-iodobenzoate as a solid that was taken to the next step without further purification. LCMS (ES, m/z): 359 [M+H]⁺

### Synthesis of Intermediate C35

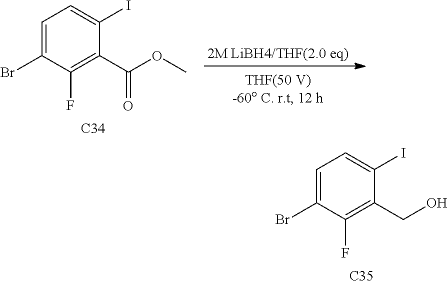

Into a 250-mL 3-necked round-bottom flask purged and maintained with an inert atmosphere of nitrogen, was placed methyl 3-bromo-2-fluoro-6-iodobenzoate (2.0 g, 5.58 mmol, 1.00equiv), THE (100 mL). This was the followed by the addition 2M LiBH4 in THE (2.0 eq) added dropwise with stirring at −60° C. The resulting mixture was allowed to warm and stirred for 1 overnight at room temperature. The reaction was then quenched by the addition of 100 mL of water. The resulting solution was extracted with 3×100 mL of ethyl acetate and the organic layers combined. The resulting mixture was washed with 1×200 ml of saturated aqueous NaCl. The mixture was dried over anhydrous sodium sulfate. The solids were filtered out. The resulting mixture was concentrated under vacuum. The residue was applied onto a silica gel column with ethyl acetate/petroleum ether. This resulted in 900 mg of (5-bromo-4-fluoro-2-iodophenyl)methanol as a solid that was taken to the next step without further purification. LCMS (ES, m/z): 331 [M+H]⁺

### Synthesis of Intermediate C36

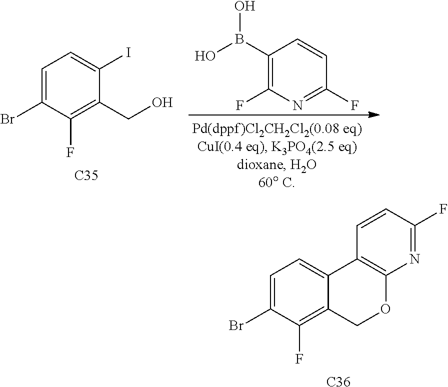 [SMILES: OCc1c(I)ccc(Br)c1F ; OB(O)c1ccc(F)nc1F ; "C35" ; Fc1ccc2c(n1)OCc1c-2ccc(Br)c1F ; "C36"]

Into a 40 mL vial, were added (5-bromo-4-fluoro-2-iodophenyl)methanol (500 mg, 1.51 mmol, 1.00 equiv), (2,6-difluoropyridin-3-yl)boronic acid (481.81 mg, 3.03 mmol, 2.00 equiv), dioxane (12 mL), K₃po₄ (803.03 mg, 3.78 mmol, 2.50 equiv)/H₂O (3 mL) and Pd(dppf)Cl₂·CH₂Cl₂ (98.90 mg, 0.12 mmol, 0.08 equiv), CuI (115.15 mg, 0.60 mmol, 0.40 equiv). Then the reaction mixture was evacuated and flushed three times with nitrogen. The resulting mixture was stirred overnight at 60° C. The reaction was then quenched by the addition of 20 mL of water. The resulting solution was extracted with 3×20 mL of ethyl acetate and the organic layers combined. The resulting mixture was washed with 1×50 ml of saturated aqueous NaCl. The mixture was dried over anhydrous sodium sulfate. The solids were filtered out. The resulting mixture was concentrated under vacuum. The residue was applied onto a silica gel column with ethyl acetate/petroleum ether. This resulted in 300 mg of 8-bromo-3,7-difluoro-6H-isochromeno[3,4-b]pyridine (C36) as a solid that was taken to the next step without further purification. LCMS (ES, m/z): 298 [M+H]⁺

### 5. Synthesis of Intermediate C37

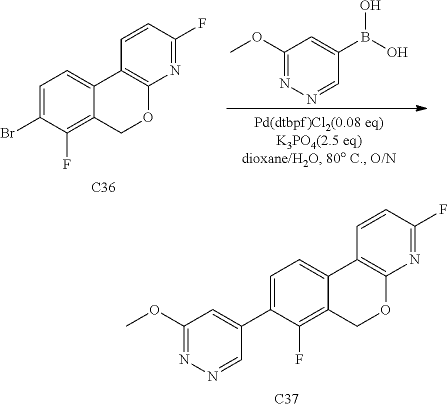

Into a 40 mL vial, were added 8-bromo-3,7-difluoro-6H-isochromeno[3,4-b]pyridine (150 mg, 0.50 mmol, 1.00 equiv), (6-methoxypyridazin-4-yl)boronic acid (93.33 mg, 0.60 mmol, 1.2 equiv), dioxane (3 mL), K₃PO₄ (267.67 mg, 1.26 mmol, 2.50 equiv)/H₂O (0.6 mL) and Pd(dtbpf)Cl₂ (26.30 mg, 0.04 mmol, 0.08 equiv). Then the reaction mixture was evacuated and flushed three times with nitrogen. The resulting mixture was stirred overnight at 80° C. The reaction mixture was poured into 40 mL of water. The isolated solid was collected. The resulting mixture was concentrated under vacuum. This resulted in 160 mg of 3,7-difluoro-8-(6-methoxypyridazin-4-yl)-6H-isochromeno[3,4-b]pyridine (C37) as a solid that was taken to the next step without further purification. LCMS (ES, m/z): 328 [M+H]⁺

### Synthesis of Intermediate c38

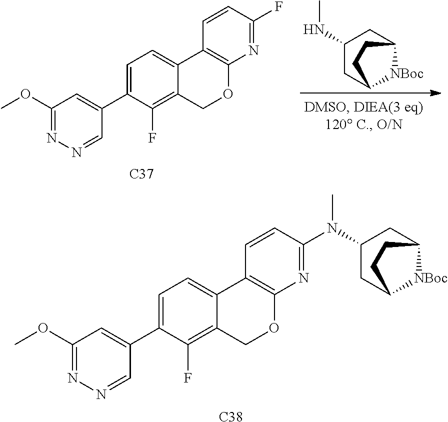

Into a 8-mL vial, was placed 3,7-difluoro-8-(6-methoxypyridazin-4-yl)-6H-isochromeno[3,4-b]pyridine (80.0 mg, 0.24 mmol, 1.00 equiv), DMSO (2 mL), tert-butyl (1R,3s,5S)-3-(methylamino)-8-azabicyclo[3.2.1]octane-8-carboxylate (88.07 mg, 0.366 mmol, 1.50 equiv), DIEA (94.67 mg, 0.73 mmol, 3.00 equiv). The resulting solution was stirred overnight at 120° C. The mixture was allowed to cool down to room temperature. The reaction was then quenched by the addition of 10 mL of water. The resulting solution was extracted with 3×10 mL of ethyl acetate and the organic layers combined. The resulting mixture was washed with 3×20 ml of half saturated aqueous NaCl and 1×20 ml of saturated aqueous NaCl. The mixture was dried over anhydrous sodium sulfate. The solids were filtered out. The resulting mixture was concentrated under vacuum. The crude product was purified by Prep-HPLC with the following conditions: Mobile Phase A: Water (10 mmol/L NH₄HCO₃), Mobile Phase B: ACN. This resulted in 20 mg of tert-butyl (1R,3s,5S)-3-((7-fluoro-8-(6-methoxypyridazin-4-yl)-6H-isochromeno[3,4-b]pyridin-3-yl)(methyl)amino)-8-azabicyclo[3.2.1]octane-8-carboxylate (C38) as a solid that was taken to the next step without further purification. LCMS (ES, m/z): 548 [M+H]⁺

### Synthesis of Compound 245

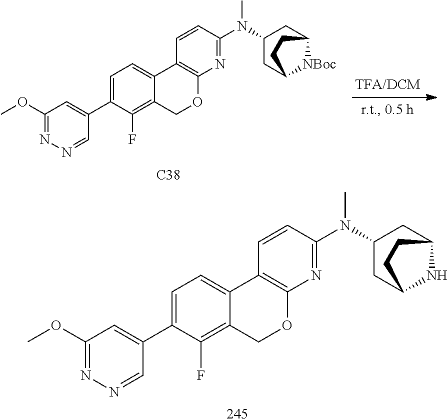

Into a 25-mL round-bottom flask, was tert-butyl (1R,3s,5S)-3-((7-fluoro-8-(6-methoxypyridazin-4-yl)-6H-isochromeno[3,4-b]pyridin-3-yl)(methyl)amino)-8-azabicyclo[3.2.1]octane-8-carbOXylate (20.0 MG, 0.036 mmol, 1.00 equiv), DCM (1 mL), TFA (0.2 mL). The resulting solution was stirred for 30 min at room temperature. The resulting mixture was concentrated under vacuum. The crude product was purified by Prep-HPLC (Condition 2, Gradient 13). This resulted in 5.2 mg of N-((1R,3s,5S)-8-azabicyclo[3.2.1]octan-3-yl)-7-fluoro-8-(6-methoxypyridazin-4-yl)-N-methyl-6H-isoch-romeno[3,4-b]pyridin-3-amine (Compound 245) as a solid that was taken to the next step without further purification. LCMS (ES, m/z): 448 [M+H]^{+ 1}H-NMR (400 MHz, DMSO-d₆) δ 9.16 (t, J=1.8 Hz, 1H), 8.09 (d, J=8.7 Hz, 1H), 7.72 (t, J=8.0 Hz, 1H), 7.64 (d, J=8.3 Hz, 1H), 7.41 (dd, J=1.9, 0.9 Hz, 1H), 6.41 (d, J=8.7 Hz, 1H), 5.40 (s, 2H), 4.89 (s, 1H), 4.09 (s, 3H), 3.50 (s, 2H), 2.84 (s, 3H), 1.72 (dt, J=11.2, 8.7 Hz, 6H), 1.53-1.39 (m, 2H).

### Example 64: Synthesis of Compound 246

### Synthesis of Intermediate C39

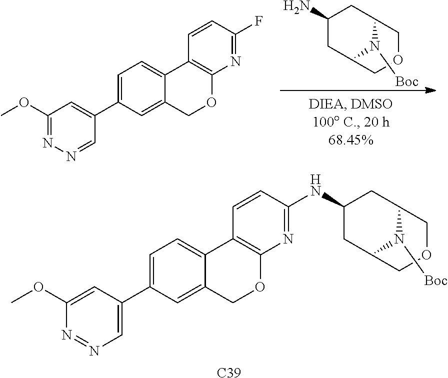 [SMILES: COc1cc(-c2ccc3c(c2)COc2nc(F)ccc2-3)cnn1 ; CC(C)(C)OC(=O)N1[C@@H]2COC[C@H]1C[C@@H](N)C2 ; COc1cc(-c2ccc3c(c2)COc2nc(N[C@H]4C[C@H]5COC[C@@H](C4)N5C(=O)OC(C)(C)C)ccc2-3)cnn1 ; "C39"]

To a stirred mixture of 5-{3-fluoro-6H-isochromeno[3,4-b]pyridin-8-yl}-3-methoxypyridazine (85 mg, 0.275 mmol, 1 equiv)) and tert-butyl (1R,5S,7s)-7-amino-3-oxa-9-azabicyclo[3.3.1]nonane-9-carboxylaTE (79.91 mg, 0.330 mmol, 1.2 equiv) in DMSO (1.7 mL) were added DIEA (71.04 mg, 0.550 mmol, 2.0 equiv) dropwise at room temperature. The resulting mixture was stirred for 20 hr at 100° C. The resulting mixture was purified by silica gel column chromatography, eluted with CH₂Cl₂/MeOH (50:1) to afford tert-butyl (1R,5S,7s)-7-{[8-(6-methoxypyridazin-4-yl)-6H-isochromeno[3,4-b]pyridin-3-yl]amino}-3-oxa-9-azabicyclo[3.3.1]nonane-9-carboxylate (C39, 100 mg, 68%) as a solid that was taken to the next step without further purification. LCMS (ES, m/z): 532[M+H]⁺

### Synthesis of Intermediate C40

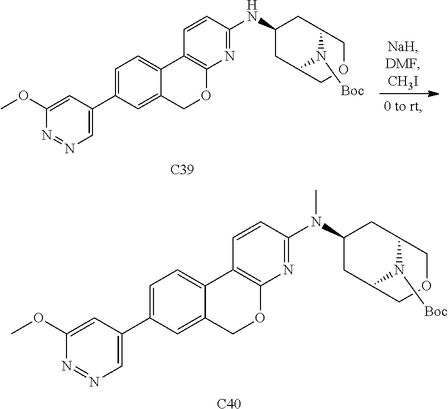 [SMILES: COc1cc(-c2ccc3c(c2)COc2nc(N[C@H]4C[C@H]5COC[C@@H](C4)N5C(=O)OC(C)(C)C)ccc2-3)cnn1 ; "C39" ; COc1cc(-c2ccc3c(c2)COc2nc(N(C)[C@H]4C[C@H]5COC[C@@H](C4)N5C(=O)OC(C)(C)C)ccc2-3)cnn1 ; "C40"]

To a solution of tert-butyl (1R,5S,7s)-7-{[8-(6-methoxypyridazin-4-yl)-6H-isochromeno[3,4-b]pyridin-3-yl]amino}-3-oxa-9-azabicyclo[3.3.1]nonane-9-cARboxylate (95 mg, 0.179 mmol, 1 equiv) in DMF (2 mL, 25.844 mmol, 144.62 equiv) was added sodium hydride (60% in oil, 14.29 mg) at 0° C. The mixture was stirred for 15 min. Methyl iodide (22.83 mg, 0.161 mmol, 0.9 equiv) was added dropwise and the mixture was allowed to warm to room temperature and stirred for 2 hr. The reaction mixture was quenched by water and extracted with DCM (3×20 mL). The organic phase was concentrated under reduced pressure. The residue was purified by silica gel column chromatography, eluted with PE/EA (1:1) to afford tert-butyl (1R,5S,7s)-7-{[8-(6-methoxypyridazin-4-yl)-6H-isochromeno[3,4-b]pyridin-3-yl](methyl)amino}-3-oxa-9-azabicyclo[3.3.1]nonane-9-carboxylate (C40, 80 mg, 82%) as a solid that was taken to the next step without further purification. LCMS (ES, m/z): 546[M+H]⁺

### Synthesis of Compound 246

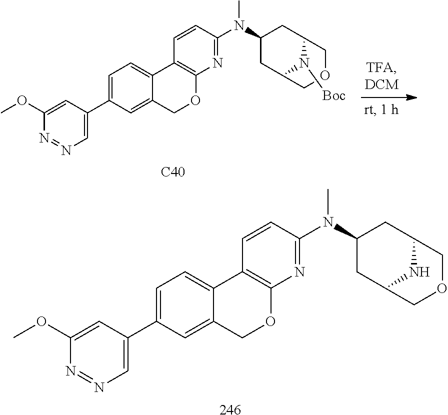 [SMILES: COc1cc(-c2ccc3c(c2)COc2nc(N(C)[C@H]4C[C@H]5COC[C@@H](C4)N5C(=O)OC(C)(C)C)ccc2-3)cnn1 ; "C40" ; COc1cc(-c2ccc3c(c2)COc2nc(N(C)[C@H]4C[C@H]5COC[C@@H](C4)N5)ccc2-3)cnn1 ; "246"]

To a stirred solution of tert-butyl (1R,5S,7s)-7-({8-[(3E)-4-amino-4-methoxybuta-1,3-dien-2-yl]-6H-isochromeno[3,4-b]pyridin-3-yl}(methyl)amino)-3-oxa-9-azabicyclo[3.3.1]nonane-9-carBOxylate (80 mg, 0.150 mmol, 1 equiv) in DCM (1.0 Ml, 15.730 mmol, 105.13 equiv) was added TFA (1.0 mL, 13.463 mmol, 89.98 equiv) at room temperature. The resulting mixture was stirred for 1 hr at room temperature. The resulting mixture was concentrated under reduced pressure. The crude product was purified by Prep-HPLC (Condition 7, Gradient 3) to afford (1R,5S,7s)-N-{8-[(3E)-4-amino-4-methoxybuta-1,3-dien-2-yl]-6H-isochromeno[3,4-b]pyridin-3-yl}-3-oxa-9-azabicyclo[3.3.1]nonan-7-amine (Compound 246, 9.2 mg, 14%) as a solid. LCMS (ES, m/z): 446[M+H]+¹H NMR (400 MHz, DMSO-d6) δ 9.34 (d, J=1.9 Hz, 1H), 8.10 (d, J=8.7 Hz, 1H), 7.90 (dd, J=8.2, 2.0 Hz, 1H), 7.85-7.76 (m, 2H), 7.51 (d, J=1.9 Hz, 1H), 6.53 (d, J=8.6 Hz, 1H), 5.29 (s, 2H), 4.77 (s, 1H), 4.09 (s, 3H), 3.56-3.44 (m, 4H), 3.03 (d, J=10.3 Hz, 2H), 2.91 (s, 3H), 1.87 (td, J=11.2, 10.8, 5.7 Hz, 2H), 1.70 (td, J=12.8, 3.2 Hz, 2H).

### Example 65: Synthesis of Compound 247

### Synthesis of Intermediate C41

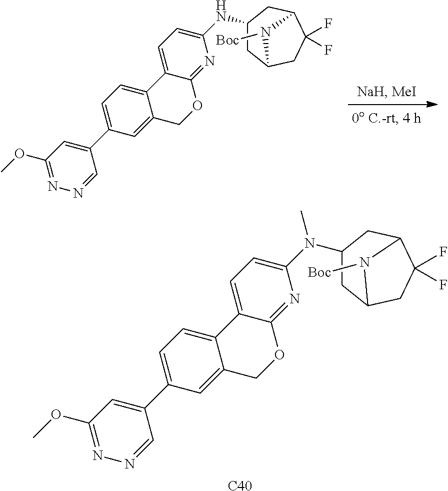 [SMILES: COc1cc(-c2ccc3c(c2)COc2nc(N[C@H]4C[C@@H]5CC(F)(F)[C@H](C4)N5C(=O)OC(C)(C)C)ccc2-3)cnn1 ; COc1cc(-c2ccc3c(c2)COc2nc(N(C)C4CC5CC(F)(F)C(C4)N5C(=O)OC(C)(C)C)ccc2-3)cnn1 ; "C40"]

To a solution of tert-butyl (1R,3S,5S)-6,6-difluoro-3-{[8-(6-methoxypyridazin-4-yl)-6H-isochromeno[3,4-b]pyridin-3-yl]amino}-8-azabicyclo[3.2.1]octane-8-carboXYlate (45 mg, 0.082 mmol, 1.0 equiv) in DMF (0.5 mL) was added NaH (2.2 mg, 0.090 mmol, 2.0 equiv) at 0° C. The mixture was stirred for 15 min. Methyl iodide (7.7 mg, 0.054 mmol, 1.2 equiv) was added and the mixture was allowed to warm to room temperature and stirred for 4 hr. The reaction mixture was quenched by water and extracted with EA (3×2 mL). The combined organic layers were washed with water (3×3 mL), dried over anhydrous Na₂SO₄. After filtration, the filtrate was concentrated under reduced pressure. The residue was purified by silica gel column chromatography, eluted with PE/EA (1:1) to afford tert-butyl 6,6-difluoro-3-{[8-(6-methoxypyridazin-4-yl)-6H-isochromeno[3,4-b]pyridin-3-yl](methyl)amino}-8-azabicyclo[3.2.1]octane-8-carboxylate (C40, 17 mg, 36%) as a solid. LCMS (ES, m/z): 566 [M+H]⁺

### Synthesis of Compound 247

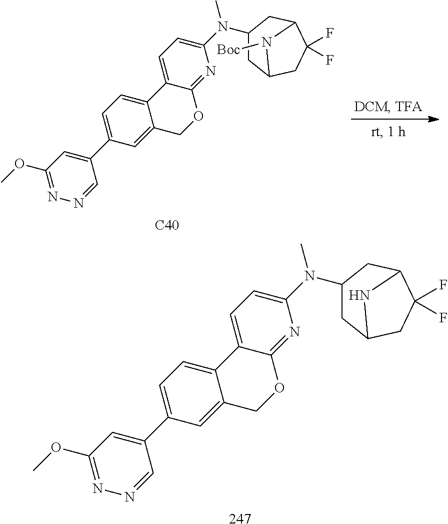 [SMILES: COc1cc(-c2ccc3c(c2)COc2nc(N(C)C4CC5CC(F)(F)C(C4)N5C(=O)OC(C)(C)C)ccc2-3)cnn1 ; "C40" ; COc1cc(-c2ccc3c(c2)COc2nc(N(C)C4CC5CC(F)(F)C(C4)N5)ccc2-3)cnn1 ; "247"]

To a stirred mixture of tert-butyl (1R,3S,5S)-6,6-difluoro-3-{[8-(6-methoxypyridazin-4-yl)-6H-isochromeno[3,4-b]pyridin-3-yl](methyl)amino}-8-azabicyclo[3.2.1]octane-8-carbOXylate (27 mg, 0.048 mMOl, 1.00 equiv) in DCM (1 mL) was added TFA (0.3 mL) dropwise at room temperature. The resulting mixture was stirred for 1 hr at room temperature. The resulting mixture was concentrated under vacuum. The crude product was purified by Prep-HPLC (Condition 6, Gradient 3) to afford (1R,3S,5S)-6,6-difluoro-N-[8-(6-methoxypyridazin-4-yl)-6H-isochromeno[3,4-b]pyridin-3-yl]-N-methyl-8-azabicyclo[3.2.1]octan-3-amine (Compound 247, 5.8 mg, 26%) as a solid. LCMS (ES, m/z): 466.2 [M+H]^{+ 1}H NMR (400 MHz, DMSO-d₆) δ 9.59-9.58 (m, 2H), 9.34 (d, J=1.9 Hz, 1H), 8.17 (d, J=8.7 Hz, 1H), 7.92 (dd, J=8.1, 2.0 Hz, 1H), 7.87-7.79 (m, 2H), 7.52 (d, J=2.0 Hz, 1H), 6.45 (d, J=8.6 Hz, 1H), 5.32 (s, 2H), 5.18-5.17 (m, 1H), 4.38-4.33 (m, 2H), 4.09 (s, 3H), 2.91 (s, 3H), 2.79-2.68 (m, 2H), 2.24 (t, J=12.7 Hz, 1H), 2.12 (t, J=13.0 Hz, 1H), 1.98 (d, J=14.4 Hz, 1H), 1.89 (d, J=12.2 Hz, 1H).

### Example 66: Synthesis of Compound 248

### Synthesis of Intermediate C41

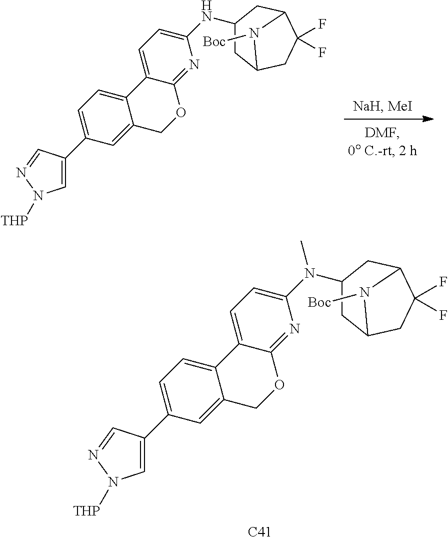

To a stirred mixture of tert-butyl 6,6-difluoro-3-({8-[1-(oxan-2-yl)pyrazol-4-yl]-6H-isochromeno[3,4-b]pyridin-3-yl}amino)-8-azabicyclo[3.2.1]octane-8-carboxyLAte (15.0 mg, 0.025 mmol, 1.0 equiv) in DMF (0.5 mL) was added NaH (1.2 mg, 0.050 mmol, 2.0 equiv) at 0 degrees C. under nitrogen atmosphere. The resulting mixture was stirred for 5 min at 0° C. under nitrogen atmosphere. To the above mixture was added CH₃I (7.2 mg, 0.050 mmol, 2.0 equiv) dropwise at 0° C. The resulting mixture was stirred for additional 2 hr at room temperature. The resulting mixture was diluted with water (2 mL) and extracted with CH₂Cl₂ (3×2 mL). The combINed organic layers were washed with water (1×3 mL), dried over anhydrous Na₂SO₄. After filtration, the filtrate was concentrated under reduced pressure. This resulted in tert-butyl 6,6-difluoro-3-[methyl({8-[1-(oxan-2-yl)pyrazol-4-yl]-6H-isochromeno [3,4-b]pyridin-3-yl}) amino]-8-azabicyclo[3.2.1]octane-8-carboxylate (C41, 15 mg, 97%) as an oil that was taken to the next step without further purification. LCMS (ES, m/z): 594 [M+H]⁺

### Synthesis of Compound 248

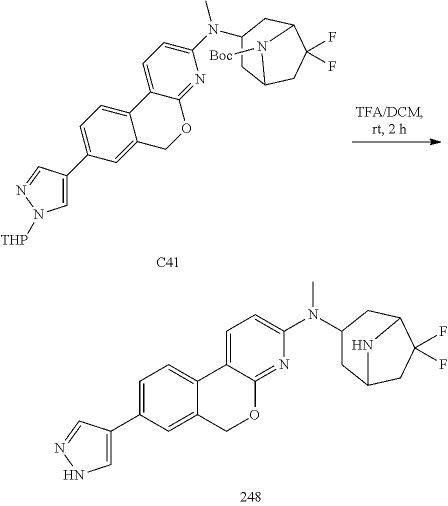

To a stirred mixture of tert-butyl 6,6-difluoro-3-[methyl({8-[1-(oxan-2-yl)pyrazol-4-yl]-6H-isochromeno[3,4-b]pyridin-3-yl})amino]-8-azabicyclo[3.2.1]octane-8-carboxYLate (15.0 mg, 0.025 mMOl, 1.0 equiv) in DCM (1 mL) was added TFA (0.3 mL) dropwise at 5° C. The resulting mixture was stirred for 2 hr at room temperature. The resulting mixture was concentrated under vacuum. The crude product was purified by Prep-HPLC (Condition 6, Gradient 5) to afford 6,6-difluoro-N-methyl-N-[8-(1H-pyrazol-4-yl)-6H-isochromeno[3,4-b]pyridin-3-yl]-8-azabicyclo[3.2.1]octan-3-amine (Compound 248, 3.5 mg, 33%) as a solid. LCMS (ES, m/z): 424 [M+H]^{+ 1}H NMR (400 MHz, Acetonitrile-d₃) δ 8.00 (d, J=8.5 Hz, 1H), 7.97-7.89 (m, 2H), 7.66-7.52 (m, 2H), 7.42 (d, J=1.7 Hz, 1H), 6.40 (d, J=8.5 Hz, 1H), 5.40-5.33 (m, 1H), 5.29 (s, 2H), 4.41-4.38 (m, 1H), 4.24 (d, J=12.3 Hz, 1H), 2.95 (s, 3H), 2.91-2.79 (m, 3H), 2.70-2.59 (m, 2H), 2.45-2.30 (m, 2H), 2.15-2.12 (m, 1H).

### Example 67: Synthesis of Compound 249

### Synthesis of Intermediate C42

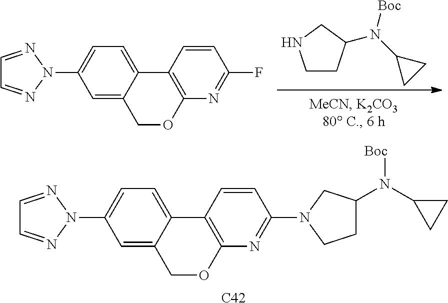 [SMILES: Fc1ccc2c(n1)OCc1cc(-n3nccn3)ccc1-2 ; CC(C)(C)OC(=O)N(C1CC1)C1CCNC1 ; CC(C)(C)OC(=O)N(C1CC1)C1CCN(c2ccc3c(n2)OCc2cc(-n4nccn4)ccc2-3)C1 ; "C42"]

Into an 8 mL vial was added 2-{3-fluoro-6H-isochromeno[3,4-b]pyridin-8-yl}-1,2,3-triazole (60 mg, 0.224 mmol, 1.00 equiv), MeCN (1 mL), tert-butyl N-cyclopropyl-N-(pyrrolidin-3-yl)carbamate (60.75 mg, 0.269 mmol, 1.2 equiv) and K₂CO₃ (92.74 mg, 0.672 mmol, 3.0 equiv) at room temperature. The resulting mixture was stirred for 6 hr at 80° C. The mixture was filtered. The filtrate was concentrated under reduced pressure to afford tert-butyl N-cyclopropyl-N-{1-[8-(1,2,3-triazol-2-yl)-6H-isochromeno[3,4-b]pyridin-3-yl]pyrrolidin-3-yl}carbamate (C42, 65 mg, 61%) as an oil. LCMS (ES, m/z): 475 [M+H]⁺

### Synthesis of Compound 249

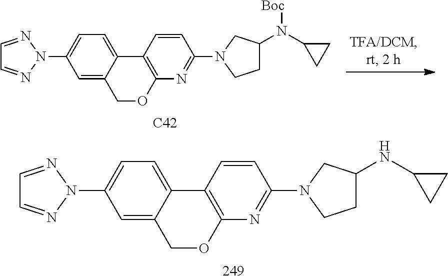

Into an 8 mL vial was added tert-butyl N-cyclopropyl-N-{1-[8-(1,2,3-triazol-2-yl)-6H-isochromeno[3,4-b]pyridin-3-yl]pyrrolidin-3-YL}carbamate (60 mg, 0.126 mmol, 1 equiv), DCM (1 mL, 15.731 mmol, 124.42 equiv) and TFA (1 mg, 0.009 mmol, 0.07 equiv) at room temperature. The resulting mixture was stirred for 2 hr at room temperature. The resulting mixture was concentrated under reduced pressure. The crude product was purified by reverse phase flash with the following conditions (Condition 8, Gradient 1) to afford N-cyclopropyl-1-[8-(1,2,3-triazol-2-yl)-6H-isochromeno[3,4-b]pyridin-3-yl]pyrrolidin-3-amine (Compound 249, 18 mg, 38%) as a solid. LCMS (ES, m/z): 375 [M+H]^{+ 1}H NMR (300 MHz, DMSO-d6) δ 8.12 (s, 2H), 8.03 (d, J=8.5 Hz, 1H), 8.00-7.87 (m, 2H), 7.79 (d, J=8.5 Hz, 1H), 6.22 (d, J=8.5 Hz, 1H), 5.31 (s, 2H), 3.58 (dd, J=10.6, 6.0 Hz, 1H), 3.47 (s, 2H), 3.48-3.37 (m, 1H), 3.23 (dd, J=10.5, 4.8 Hz, 1H), 2.14-2.05 (m, 2H), 1.87 (dt, J=12.5, 6.6 Hz, 1H), 0.40 (dd, J=6.6, 1.6 Hz, 2H), 0.24 (s, 2H).

### Example 68: Synthesis of Compound 250

### Synthesis of Compound 250

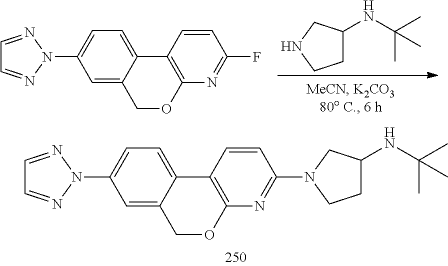

Into an 8 mL vial were added 2-{3-fluoro-6H-isochromeno[3,4-b]pyridin-8-yl}-1,2,3-Triazole (40 mg, 0.149 mmol, 1.0 equiv), MeCN (1 mL, 19.024 mmol, 127.58 equiv), N-tert-butylpyrrolidin-3-amine (25.45 mg, 0.179 mmol, 1.2 equiv) and K₂CO₃ (61.82 mg, 0.447 mmol, 3.0 equiv) at room temperature. The resulting mixture was stirred for 6 hr at 80° C. The mixture was filtered. The filtrate was concentrated under reduced pressure. The crude product was purified by reversed-phase flash (Condition 8, Gradient 1) to afford N-tert-butyl-1-[8-(1,2,3-triazol-2-yl)-6H-isochromeno[3,4-b]pyridin-3-yl]pyrrolidin-3-amine 2,2,2-trifluoroacetate (Compound 250, 9.3 mg, 12%) as a solid. LCMS (ES, m/z): 489 [M+H]^{+ 1}H NMR (300 MHz, DMSO-d6) δ 8.66 (s, 2H), 8.12 (d, J=7.6 Hz, 3H), 8.03-7.90 (m, 2H), 7.85 (d, J=8.5 Hz, 1H), 6.35 (d, J=8.5 Hz, 1H), 5.34 (s, 2H), 4.12 (s, 1H), 3.87 (dd, J=11.4, 6.9 Hz, 1H), 3.52 (dd, J=12.0, 7.2 Hz, 3H), 2.43 (s, 1H), 2.18 (dt, J=12.9, 7.4 Hz, 1H), 1.36 (s, 9H).

### Example 69: Synthesis of Compound 251

### Synthesis of Compound 251

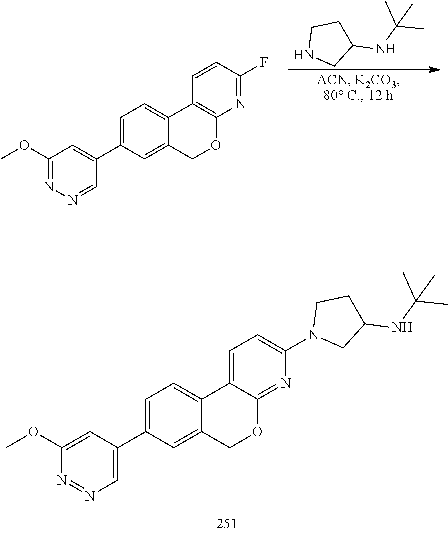

To a stirred solution of 5-{3-fluoro-6H-isochromeno[3,4-b]pyridin-8-yl}-3-methoxypyridazine (30 mg, 0.097 mmol, 1 equiv) and N-tert-butylpyrrolidin-3-amine (13.80 mg, 0.097 mmol, 1 equiv) in ACN (0.5 mL) was added K₂CO₃ (40.21 mg, 0.291 mmol, 3 equiv) at room temperature. The resulting mixture was stirred for 12 hr at 80° C. The resulting mixture was filtered and the filtrate was concentrated under reduced pressure. The crude product was purified by reverse phase flash (Condition 7, Gradient 4) to afford N-tert-butyl-1-[8-(6-methoxypyridazin-4-yl)-6H-isochromeno[3,4-b]pyridin-3-yl]pyrrolidin-3-amine (Compound 251, 6 mg, 14.33%) as a solid. LCMS (ES, m/z): 432[M+H]^{+ 1}H NMR (400 MHz, DMSO-d6) δ 9.33 (d, J=1.9 Hz, 1H), 8.07 (d, J=8.6 Hz, 1H), 7.90 (dd, J=8.2, 2.0 Hz, 1H), 7.82 (d, J=1.9 Hz, 1H), 7.78 (d, J=8.3 Hz, 1H), 7.50 (d, J=1.9 Hz, 1H), 6.22 (d, J=8.5 Hz, 1H), 5.27 (s, 2H), 4.08 (s, 3H), 3.72-3.63 (m, 1H), 3.55-3.43 (m, 2H), 2.97 (dd, J=10.4, 7.2 Hz, 1H), 2.13 (d, J=8.9 Hz, 1H), 1.77-1.63 (m, 1H), 1.08 (s, 9H).

### Example 70: Synthesis of Compound 252

### Synthesis of Intermediate C50

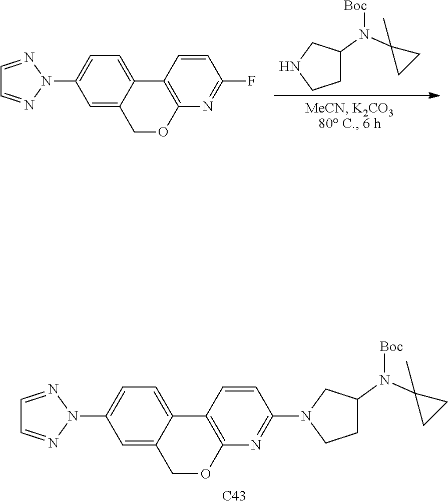

Into an 8 mL vial were added 2-{3-fluoro-6H-isochromeno[3,4-b]pyridin-8-yl}-1,2,3-triazole (50 mg, 0.186 mmol, 1.00 equiv), MeCN (1 ml), tert-butyl N-(1-methylcyclopropyl)-N-(pyrrolidin-3-yl)carbamate (53.76 mg, 0.223 mmol, 1.2 equiv) and K₂CO₃ (77.28 mg, 0.558 mmol, 3.0 equiv) at room temperature. The resulting mixture was stirred for 1 day at 80° C. The mixture was filtered. The filtrate was concentrated under reduced pressure to afford tert-butyl N-(1-methylcyclopropyl)-N-{1-[8-(1,2,3-triazol-2-yl)-6H-isochromeno[3,4-b]pyridin-3-yl]pyrrolidin-3-yl}carbamate (C50, 80 mg, 87%) as a solid that was taken to the next step without further purification. LCMS (ES, m/z): 489 [M+H]⁺

### Synthesis of Compound 252

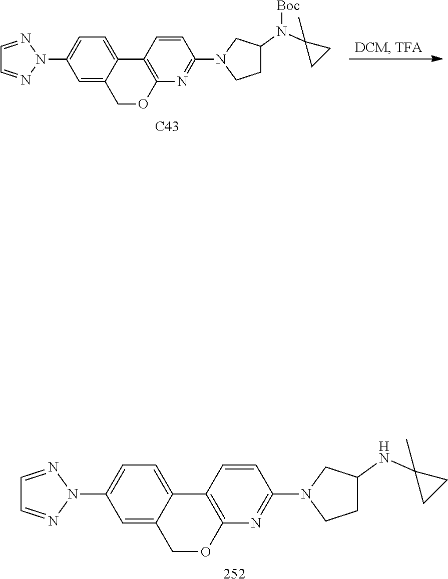

Into an 8 mL vial were added tert-butyl N-(1-methylcyclopropyl)-N-{1-[8-(1,2,3-triazol-2-yl)-6H-isochromeno[3,4-b]pyridin-3-yl]pyrrolidin-3-yl}carbamate (70 mg, 0.143 mmol, 1 equiv), DCM (1 mL, 15.731 mmol, 109.80 equiv) and TFA (1 mL, 13.463 mmol, 93.97 equiv) at room temperature. The resulting mixture was stirred for 3 h at room temperature. The resulting mixture was concentrated under reduced pressure. The residue was purified by reversed-phase flash to afford N-(1-methylcyclopropyl)-1-[8-(1,2,3-triazol-2-yl)-6H-isochromeno[3,4-b]pyridin-3-yl]pyrrolidin-3-amine (Compound 252, 0.8 mg, 8%) as a solid. LCMS (ES, m/z): 389 [M+H]^{+ 1}H NMR (300 MHz, DMSO-d6) δ 8.11 (s, 2H), 8.06-7.92 (m, 2H), 7.90 (d, J=2.2 Hz, 1H), 7.78 (d, J=8.5 Hz, 1H), 6.21 (d, J=8.5 Hz, 1H), 5.30 (s, 2H), 3.59 (s, 5H), 2.28 (s, 1H), 2.08 (dd, J=11.8, 6.1 Hz, 1H), 1.81 (dt, J=12.1, 6.3 Hz, 1H), 1.24 (s, 3H), 0.54-0.39 (m, 2H), 0.39-0.25 (m, 2H).

### Example 71: Synthesis of Compound 253

### Synthesis of Intermediate C44

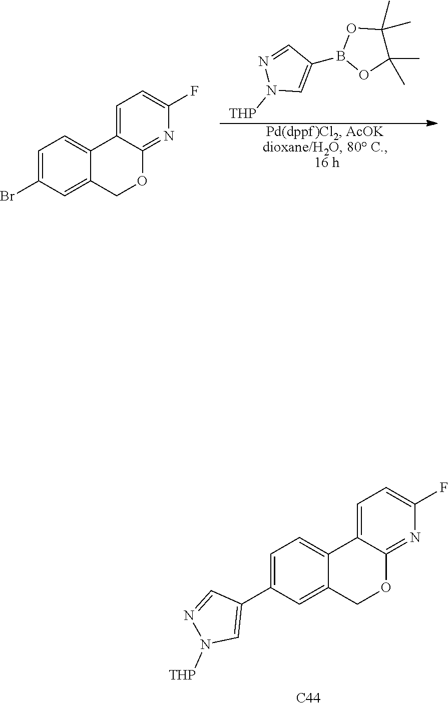

Into an 8 mL vial was added 8-bromo-3-fluoro-6H-isochromeno[3,4-b]pyridine (100 mg, 0.357 mmol, 1.00 equiv), dioxane (1.0 mL), water (0.2 mL), 1-(oxan-2-yl)-4-(4,4,5,5-tetramethyl-1,3,2-dioxaborolan-2-yl) pyrazole (119.17 mg, 0.428 mmol, 1.2 equiv), AcOK (105.12 mg, 1.071 mmol, 3.0 equiv) and Pd(dppf)Cl₂CH₂Cl₂ (29.08 mg, 0.036 mmol, 0.1 equiv) at room temperature under nitrogen atmosphere. The resulting mixture was stirred for 16 hr at 80° C. under nitrogen atmosphere. The mixture was diluted With water (10 mL) and extracted with EtOAc (3×10 mL). The combined organic layers were dried over anhydrous sodium sulfate. After filtration, the filtrate was concentrated under reduced pressure. The residue was purified by silica gel column chromatography, eluted with PE/EA (1:1) to afford 4-{3-fluoro-6H-isochromeno[3,4-b]pyridin-8-yl}-1-(oxan-2-yl)pyrazole (C44, 90 mg, 71%) as an solid that was taken to the next step without further purification. LCMS (ES, m/z): 352 [M+H]⁺

### Synthesis of Intermediate C45

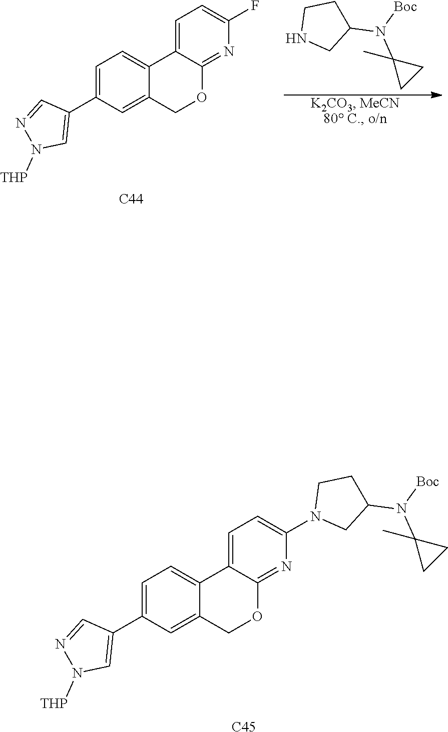

Into an 8 mL vial was added 4-{3-fluoro-6H-isochromeno[3,4-b]pyridin-8-yl}-1-(oxan-2-Yl)pyrazole (90 mg, 0.256 mmol, 1.0 equiv), MeCN (1 mL, 19.024 mmol, 74.27 equiv), tert-butyl N-(1-methylcyclopropyl)-N-(pyrrolidin-3-yl)carbamate (61.56 mg, 0.256 mmol, 1.0 equiv) and K₂CO₃ (106.20 mg, 0.768 mmol, 3.0 equiv) at room temperature. The resulting mixture was stirred overnight at 80° C. The mixture was filtered. The filtrate was concentrated under reduced pressure to afford tert-butyl N-(1-methylcyclopropyl)-N-(1-{8-[1-(oxan-2-yl)pyrazol-4-yl]-6H-isochromeno[3,4-b]pyridin-3-yl}pyrrolidin-3-yl)carbamate (C45, 90 mg, 61%) as a solid that was taken to the next step without further purification. LCMS (ES, m/z): 572 [M+H]⁺

### Synthesis of Intermediate C46

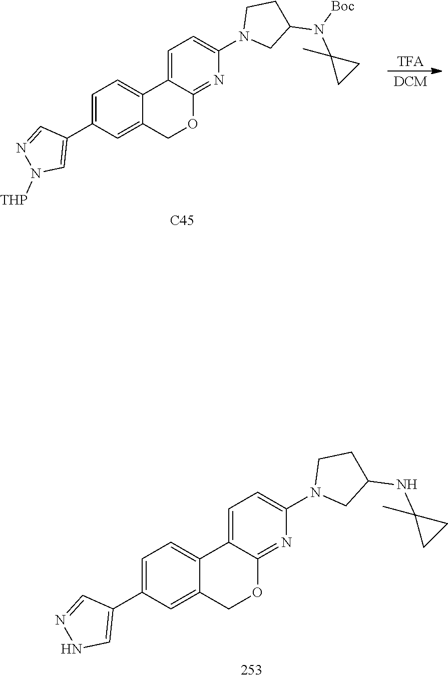

Into an 8 mL vial was added tert-butyl N-(1-methylcyclopropyl)-N-(1-{8-[1-(oxan-2-yl)pyrazol-4-yl]-6H-isochromeno[3,4-b]pyridin-3-yl}pyrrolidin-3-Yl)carbamate (90 mg, 0.157 mmol, 1.0 equiv), DCM (1 mL, 15.731 mmol, 99.93 equiv) and 2,2,2-trifluoroacetic acid (1 mL, 10.202 mmol, 64.81 equiv) at room temperature. The resulting mixture was stirred for 3 hr at room temperature. The resulting mixture was concentrated under reduced pressure.

The residue was purified by reversed-phase flash (Condition 8, Gradient 1) to afford N-(1-methylcyclopropyl)-1-[8-(1H-pyrazol-4-yl)-6H-isochromeno[3,4-b] pyridin-3-yl]pyrrolidin-3-amine (Compound 253, 12 mg, 19%) as a solid. LCMS (ES, m/z): 388 [M+H]^{+ 1}H NMR (300 MHz, DMSO-d6) δ 12.93 (s, 1H), 8.17 (s, 1H), 7.97 (d, J=8.4 Hz, 1H), 7.92 (s, 1H), 7.64-7.51 (m, 2H), 7.45 (s, 1H), 6.16 (d, J=8.4 Hz, 1H), 5.20 (s, 2H), 3.59 (d, J=7.5 Hz, 5H), 3.12 (d, J=4.6 Hz, 1H), 2.08 (s, 2H), 1.25 (s, 3H), 0.54-0.29 (m, 4H).

### Example 72: Synthesis of Compound 254

### Synthesis of Intermediate C47

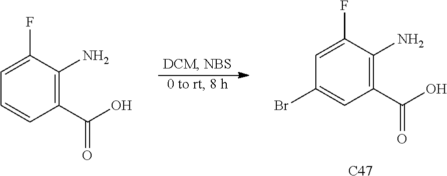

To a stirred solution of 2-amino-3-fluorobENzoic acid (15 g, 96.694 mmol, 1 equiv) in DCM (150 mL) was added NBS (16.35 g, 91.859 mmol, 0.95 equiv) in portions at 0° C. The resulting mixture was stirred for 8 hr at room temperature. The resulting mixture was concentrated under reduced pressure. The residue was purified by silica gel column chromatography, eluted with CH₂Cl₂/MeOH (25:1) to afford 2-amino-5-bromo-3-fluorobenzoic acid (C47, 21 g, 92%) as a solid that was taken to the next step without further purification. LCMS (ES, m/z): 234 [M+H]⁺

### Synthesis of Intermediate C48

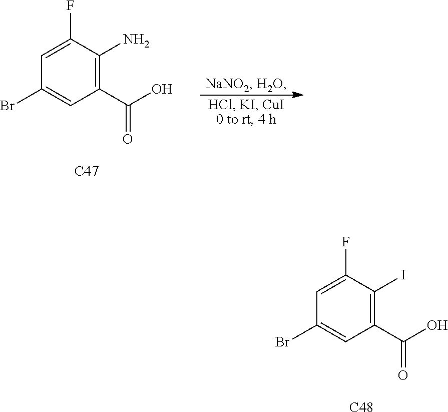 [SMILES: Nc1c(F)cc(Br)cc1C(=O)O ; "HCl, KI, CuI0 to rt, 4 h" ; "NaNO2, H2O," ; "C47" ; O=C(O)c1cc(Br)cc(F)c1I ; "C48"]

To a solution of 2-amino-5-bromo-3-fluorobENzoic acid (20 g, 85.461 mmol, 1 equiv) in HCl (172 mL, 2 mol/L) was added a solution of NaNO₂ (5.90 g, 85.461 mmol, 1 equiv) in water (86 ml) at 0° C. The resulting mixture was stirred for 1.5 h at 0° C. This solution was then added a mixture of KI (21.28 g, 128.191 mmol, 1.50 equiv) and CuI (8.14 g, 42.730 mmol, 0.5 equiv) in water (86 ml) dropwise at 0° C. The resulting mixture was stirred for 4 hr at room temperature.

The resulting mixture was filtered. The filter cake was purified by silica gel column chromatography, eluted with CH₂Cl₂/MeOH (10:1) to afford 5-bromo-3-fluoro-2-iodobenzoic acid (C48, 28 g, 85%) as a solid that was taken to the next step without further purification. LCMS (ES, m/z): 345 [M+H]⁺

### Synthesis of Intermediate C49

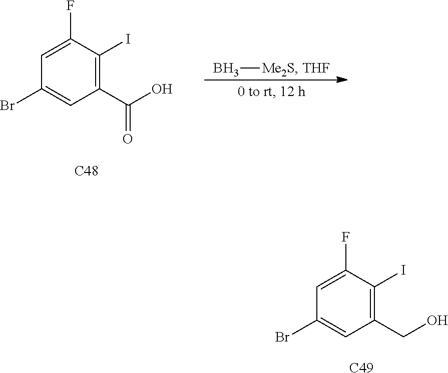

To a stirred solution of 5-bromo-3-fluoro-2-iodOBenzoic acid (5 g, 14.497 mmol, 1 equiv) in THE (50 mL) were added BH₃-Me₂S (2.20 g, 1M in THE 28.994 mmol, 2 equiv) dropwise at 0° C. The resulting mixture was stirred for 12 hr at room temperature. The reaction was quenched by the addition of saturated NH₄Cl (aq.) (30 mL) at room temperature and extracted with EtOAc (3×100 mL). The combined organic layers were washed with brine (1×30 mL), dried over anhydrous Na₂SO₄. After filtration, the filtrate was concentrated under reduced pressure. The residue was purified by silica gel column chromatography, eluted with CH₂Cl₂/MeOH (10:1) to afford (5-bromo-3-fluoro-2-iodophenyl)methanol (C49, 2.7 g, 56%) as a solid that was taken to the next step without further purification. LCMS (ES, m/z): 331 [M+H]⁺

### Synthesis of Intermediate C50

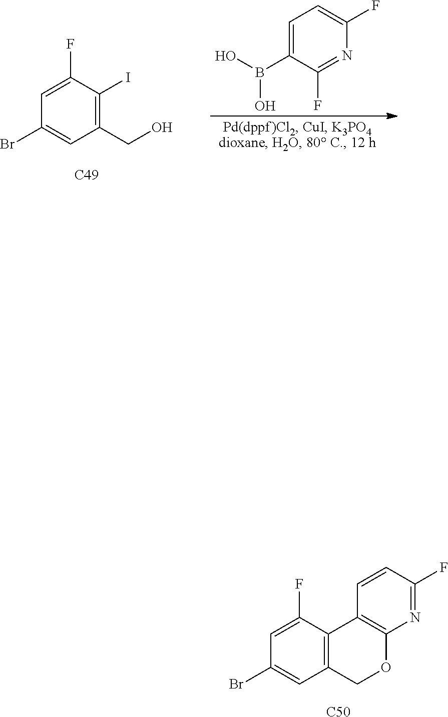

To a mixture of (5-bromo-3-fluoro-2-iodophenyl)methanol (1 g, 3.022 mmol, 1 equiv) and 2,6-difluoropyridin-3-ylboronic Acid (0.58 g, 3.626 mmol, 1.2 equiv) in dioxane (10 mL) and H₂O (2 mL) was added K₃PO₄ (1.92 g, 9.066 mmol, 3 equiv), CuI (0.06 g, 0.302 mmol, 0.1 equiv) and Pd(dppf)Cl₂ (0.22 g, 0.302 mmol, 0.1 equiv) at room temperature under nitrogen atmosphere. The resulting mixture was stirred for 12 h at 80° C. under nitrogen atmosphere. The resulting mixture was diluted with H₂O (30 mL) and extracted with EtOAc (3×50 mL). The combined organic layers were washed with brine (1×30 mL), dried over anhydrous Na₂SO₄. After filtration, the filtrate was concentrated under reduced pressure. The residue was purified by silica gel column chromatography, eluted with CH₂Cl₂/MeOH (10:1) to afford 8-bromo-3,10-difluoro-6H-isochromeno[3,4-b]pyridine (C50, 160 mg, 17%) as a solid that was taken to the next step without further purification. LCMS (ES, m/z): 298 [M+H]⁺

### Synthesis of Intermediate C51

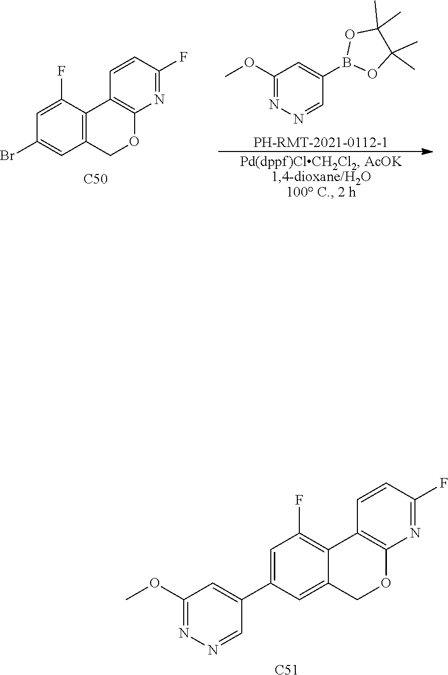

To a stirred mixture of 8-bromo-3,10-difluoro-6H-isochromeno[3,4-b]pyridine (100 mg, 0.335 mmol, 1 equiv) and 3-methoxy-5-(4,4,5,5-tetramethyl-1,3,2-dioxaborolan-2-yl)pyridazine (95.04 mg, 0.402 Mmol, 1.2 equiv) in 1,4-dioxane (1 mL) and H₂O (0.2 mL) was added AcOK (98.77 mg, 1.005 mmol, 3 equiv) and Pd(dppf)Cl₂·CH₂Cl₂ (27.33 mg, 0.034 mmol, 0.1 equiv) at room temperature under nitrogen atmosphere. The resulting mixture was stirred for 2 hr at 100° C. under nitrogen atmosphere. The resulting mixture was concentrated under reduced pressure. The residue was purified by silica gel column chromatography, eluted with CH₂Cl₂/MeOH (10:1) to afford 5-{3,10-difluoro-6H-isochromeno[3,4-b]pyridin-8-yl}-3-methoxypyridazine (C51, 50 mg, 45%) as an oil that was taken to the next step without further purification. LCMS (ES, m/z): 328 [M+H]⁺

### Synthesis of Intermediate C52

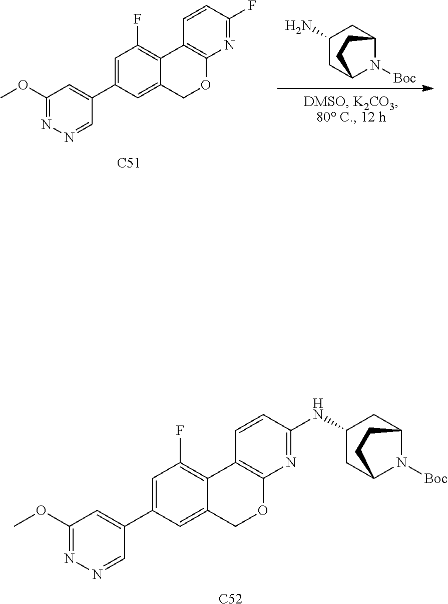

To a stirred mixture of 5-{3,10-difluoro-6H-isochromeno[3,4-b]pyridin-8-yl}-3-methoxypyridazine (50 mg, 0.153 mmol, 1 equiv) and tert-butyl (1R,3S,5S)-3-amino-8-azabicyclo[3.2.1]octane-8-carbOXylate (41.49 mg, 0.184 mmol, 1.2 equiv) in DMSO (1 mL) was added K₂CO₃ (63.34 mg, 0.459 mmol, 3 equiv) at room temperature. The resulting mixture was stirred for 12 hr at 80° C. The resulting mixture was diluted with H₂O (30 mL) and extracted with EtOAc (3×30 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford tert-butyl (1R,3S,5S)-3-{[10-fluoro-8-(6-methoxypyridazin-4-yl)-6H-isochromeno[3,4-b]pyridin-3-yl]amino}-8-azabicyclo[3.2.1]octane-8-carboxylate (C52, 70 mg, 85%) as a solid that was taken to the next step without further purification. LCMS (ES, m/z): 534[M+H]⁺

### Synthesis of Intermediate C53

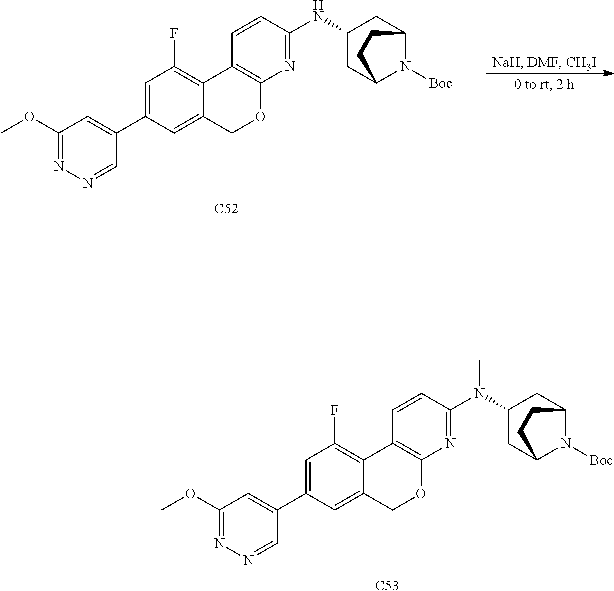

To a solution of tert-butyl (1R,3S,5S)-3-{[10-fluoro-8-(6-methoxypyridazin-4-yl)-6H-isochromeno[3,4-b]pyridin-3-yl]amino}-8-azabicyclo[3.2.1]octane-8-CArboxylate (70 mg, 0.131 mmol, 1 equiv) in DMF (0.7 mL) was added sodium hydride (60% in oil, 6.3 mg) at 0° C. The mixture was stirred for 15 min. CH₃I (16.76 mg, 0.118 mmol, 0.9 equiv) was added and the mixture was allowed to warm to room temperature and stirred for 2 hr. The reaction mixture was quenched by water (10 mL) and extracted with DCM (3×15 mL). The combined organic layers were washed with water (3×15 mL), dried over anhydrous Na₂SO₄. After filtration, the filtrate was concentrated under reduced pressure to afford tert-butyl (1R,3S,5S)-3-{[10-fluoro-8-(6-methoxypyridazin-4-yl)-6H-isochromeno[3,4-b]pyridin-3-yl](methyl)amino}-8-azabicyclo[3.2.1]octane-8-carboxylate (C53, 60 mg, 83%) as an oil that was taken to the next step without further purification. LCMS (ES, m/z): 548 [M+H]⁺

### Synthesis of Compound 254

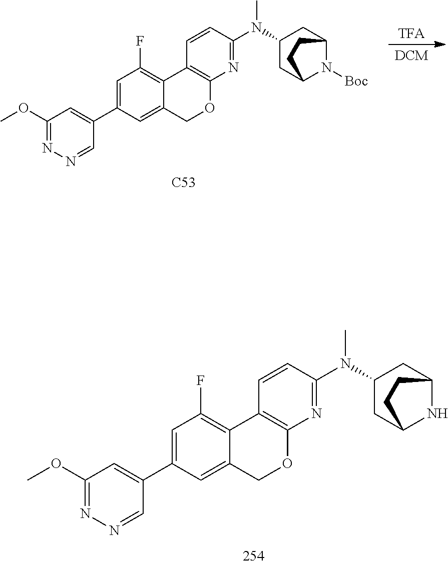

To a stirred solution of tert-butyl (1R,3S,5S)-3-{[10-fluoro-8-(6-methoxypyridazin-4-yl)-6H-isochromeno[3,4-b]pyridin-3-yl](methyl)amino}-8-azabicyclo[3.2.1]octane-8-carboxylate (60 mg, 0.110 mmol, 1 equiv) in DCM (1 mL) was added TFA (1 mL, 10.202 mmol, 93.11 equiv) at room temperature. The resulting mixture was stirred for 1 hr at room temperature and concentrated under reduced pressure. The crude product was purified by reversed-phase flash (Condition 9, Gradient 1) to afford (1R,3S,5S)—N-[10-fluoro-8-(6-methoxypyridazin-4-yl)-6H-isochromeno[3,4-b]pyridin-3-yl]-N-methyl-8-azabicyclo[3.2.1]octan-3-amine; bis(trifluoroacetaldehyde) (Compound 254, 22.9 mg, 32%) as a solid. LCMS (ES, m/z): 448[M+H]+ 1H NMR (300 MHz, DMSO-d₆) δ 9.37 (d, J=1.9 Hz, 1H), 8.60 (d, J=16.0 Hz, 2H), 8.11 (d, J=8.1 Hz, 1H), 7.90 (d, J=14.2 Hz, 1H), 7.79 (d, J=1.7 Hz, 1H), 7.59 (d, J=1.9 Hz, 1H), 6.53 (d, J=8.8 Hz, 1H), 5.29 (s, 2H), 4.99 (s, 1H), 4.09 (s, 3H), 2.88 (s, 3H), 2.13-1.95 (m, 7H), 1.71 (d, J=13.7 Hz, 2H).

### Example 73: Synthesis of Compound 255

### Synthesis of Intermediate C54

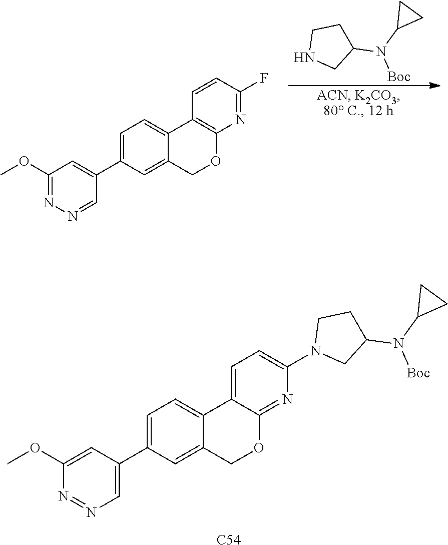

To a stirred solution of 5-{3-fluoro-6H-isochromeno[3,4-b]pyridin-8-yl}-3-methoxypyridazine (50 mg, 0.162 mmol, 1 equiv) and N-tert-butylpyrrolidin-3-aminE (13.80 mg, 0.097 mmol, 1 equiv) in acetonitrile (1 mL) was added K₂CO₃ (67.02 mg, 0.486 mmol, 3 equiv) at room temperature. The resulting mixture was stirred for 12 hr at 80° C. and concentrated under reduced pressure. The residue was purified by silica gel column chromatography, eluted with CH₂Cl₂/MeOH (20:1) to afford tert-butyl N-cyclopropyl-N-{1-[8-(6-methoxypyridazin-4-yl)-6H-isochromeno[3,4-b]pyridin-3-yl]pyrrolidin-3-yl}carbamate (C54, 70 mg, 83%) as a solid that was taken to the next step without further purification. LCMS (ES, m/z): 516[M+H]⁺

### Synthesis of Compound 255

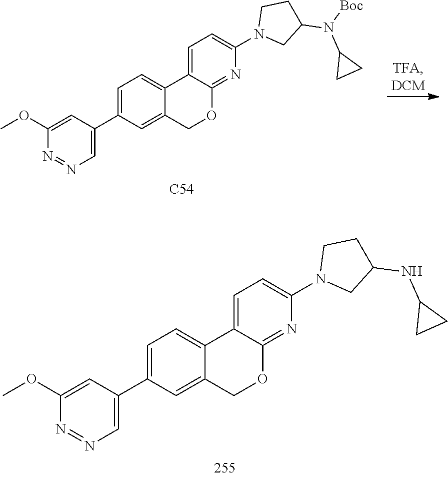 [SMILES: COc1cc(-c2ccc3c(c2)COc2nc(N4CCC(N(C(=O)OC(C)(C)C)C5CC5)C4)ccc2-3)cnn1 ; "C54" ; COc1cc(-c2ccc3c(c2)COc2nc(N4CCC(NC5CC5)C4)ccc2-3)cnn1]

To a stirred solution of tert-butyl N-cyclopropyl-N-{1-[8-(6-methoxypyridazin-4-yl)-6H-isochromeno[3,4-b]pyridin-3-yl]pyrrolidin-3-Yl}carbamate (70 mg, 0.136 mmol, 1 equiv) in DCM (1 mL) was added TFA (1 mL, 13.463 mmol, 99.17 equiv) at room temperature. The resulting mixture was stirred for 1 hr at room temperature and concentrated under reduced pressure. The crude product was purified by prep-HPLC (Condition 7, Gradient 4) to afford N-cyclopropyl-1-[8-(6-methoxypyridazin-4-yl)-6H-isochromeno[3,4-b]pyridin-3-yl]pyrrolidin-3-amine (Compound 255, 7.8 mg, 13%) as a solid. LCMS (ES, m/z): 416[M+H]+H NMR (300 MHz, DMSO-d₆) δ 9.33 (d, J=1.9 Hz, 1H), 8.07 (d, J=8.6 Hz, 1H), 7.90 (d, J=8.3 Hz, 1H), 7.85-7.73 (m, 2H), 7.50 (d, J=1.9 Hz, 1H), 6.23 (d, J=8.5 Hz, 1H), 5.27 (s, 2H), 4.08 (s, 3H), 3.58-3.42 (m, 1H), 3.42-3.34 (m, 2H), 3.32-3.23 (m, 1H), 2.14-2.05 (m, 2H), 1.86 (dd, J=12.4, 6.3 Hz, 1H), 0.44-0.34 (m, 2H), 0.33-0.24 (m, 2H).

### Example 74: Synthesis of Compound 256

### Synthesis of Intermediate C55

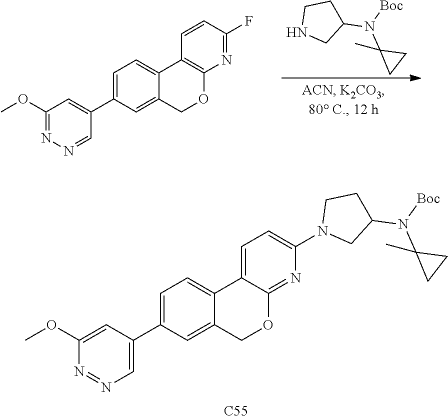 [SMILES: COc1cc(-c2ccc3c(c2)COc2nc(F)ccc2-3)cnn1 ; CC(C)(C)OC(=O)N(C1CCNC1)C1(C)CC1 ; COc1cc(-c2ccc3c(c2)COc2nc(N4CCC(N(C(=O)OC(C)(C)C)C5(C)CC5)C4)ccc2-3)cnn1 ; "C55"]

To a stirred solution of 5-{3-fluoro-6H-isochromeno[3,4-b]pyridin-8-yl}-3-methoxypyridazine (50 mg, 0.162 mmol, 1 equiv) and tert-butyl N-(1-methylcyclopropyl)-N-(pyrrolidin-3-yl)carbamate (38.85 mg, 0.162 mmol, 1 equiv) in acetonitrile (1 mL) was added K₂CO₃ (67.02 mg, 0.486 mmol, 3 equiv) at room temperature. The resulting mixture was stirred for 12 hr at 80° C. The resulting mixture was concentrated under reduced pressure. The residue was purified by silica gel column chromatography, eluted with CH₂Cl₂/MeOH (20:1) to afford tert-butyl N-{1-[8-(6-methoxypyridazin-4-yl)-6H-isochromeno[3,4-b]pyridin-3-yl]pyrrolidin-3-yl}-N-(1-methylcyclopropyl)carbamate (C55, 80 mg, 93%) as a solid that was taken to the next step without further purification. LCMS (ES, m/z): 530[M+H]⁺

### Synthesis of Compound 256

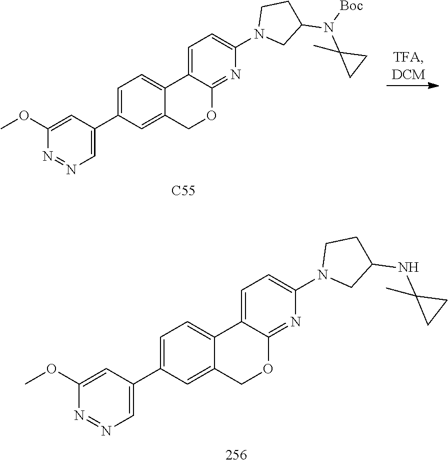 [SMILES: COc1cc(-c2ccc3c(c2)COc2nc(N4CCC(N(C(=O)OC(C)(C)C)C5(C)CC5)C4)ccc2-3)cnn1 ; "C55" ; COc1cc(-c2ccc3c(c2)COc2nc(N4CCC(NC5(C)CC5)C4)ccc2-3)cnn1 ; "256"]

To a stirred solution of tert-butyl N-{1-[8-(6-methoxypyridazin-4-yl)-6H-isochromeno[3,4-b]pyridin-3-yl]pyrrolidin-3-yl}-N-(1-methylcycloproPYl)carbamate (80 mg, 0.151 mmol, 1 equiv) in Dcm (1 mL, 15.731 mmol, 104.14 equiv) was added TFA (1 mL, 13.463 mmol, 89.13 equiv) at room temperature. The resulting mixture was stirred for 1 hr at room temperature and concentrated under reduced pressure. The crude product was purified by prep-HPLC (Condition 7, Gradient 4) to afford 1-[8-(6-methoxypyridazin-4-yl)-6H-isochromeno[3,4-b]pyridin-3-yl]-N-(1-methylcyclopropyl)pyrrolidin-3-amine (Compound 256, 8.6 mg, 13%) as a solid. LCMS (ES, m/z): 430[M+H]+¹H NMR (300 MHz, DMSO-d₆) δ 9.33 (d, J=1.9 Hz, 1H), 8.07 (d, J=8.6 Hz, 1H), 7.90 (d, J=8.6 Hz, 1H), 7.85-7.73 (m, 2H), 7.51 (d, J=1.9 Hz, 1H), 6.21 (d, J=8.5 Hz, 1H), 5.27 (s, 2H), 4.08 (s, 3H), 3.59 (s, 4H), 3.15 (s, 1H), 2.12-2.02 (m, 1H), 1.82 (s, 1H), 1.24 (d, J=2.2 Hz, 5H), 0.44 (t, J=9.5 Hz, 2H), 0.32 (s, 2H).

### Example 75: Synthesis of Compound 257

### Synthesis of Intermediate C57

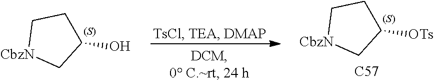

To a stirred solution of benzyl (3S)-3-hydroxypyrrolidine-1-carboxylate (4.5 g, 20.338 mmol, 1.00 equiv), DMAP (0.25 g, 2.034 mmol, 0.1 equiv) And TEA (3.09 g, 30.507 mmol, 1.5 equiv) in DCM (50 mL) was added TsCl (5.82 g, 30.507 mmol, 1.5 equiv) dropwise at 0° C. The resulting mixture was stirred for 24 hr at room temperature. The reaction was quenched with ice water (50 mL) at room temperature. The resulting mixture was extracted with CH₂Cl₂ (3×50 mL). The combined organic layers were washed with 1 M hydrochloric acid (1×50 mL), NaHCO₃ (1×50 mL) and brine (1×50 mL), dried over anhydrous Na₂SO₄. After filtration, the filtrate was concentrated under reduced pressure. The residue was purified by silica gel column chromatography, eluted with PE/EA (1:1) to afford benzyl (3S)-3-[(4-methylbenzenesulfonyl)oxy]pyrrolidine-1-carboxylate (4.68 g, 61%) as an oil that was taken to the next step without further purification. LCMS (ES, m/z): 376 [M+H]⁺

### Synthesis of Intermediate C58

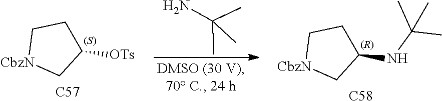

To a stirred solution of benzyl (3S)-3-[(4-methylbenzenesulfonyl)oxy]pyrrolidine-1-carboxylate (5 g, 13.318 mmol, 1.00 equiv) and erbumine (14.61 g, 199.770 mmol, 15 equiv) in DMSO (50 mL) at room temperature. The resulting mixture was stirred for 24 hr at 70° C. The Reaction was quenched by the addition of water (200 mL) at room temperature. The resulting mixture was extracted with EtOAc (3×50 mL). The combined organic layers were washed with brine (1×100 mL), dried over anhydrous Na₂SO₄. After filtration, the filtrate was concentrated under vacuum. The residue was purified by reverse flash chromatography with the following conditions: column, C18 silica gel; mobile phase, ACN in water (0.1% NH₃H₂O), 25% to 65% gradient in 12 min; detector, UV 254 nm to afford benzyl (3R)-3-(tert-butylamino)pyrrolidine-1-carboxylate (C58, 2.6 g, 70%) as an oi that was taken to the next step without further purification 1. LCMS (ES, m/z): 277 [M+H]⁺

### Synthesis of Intermediate C59

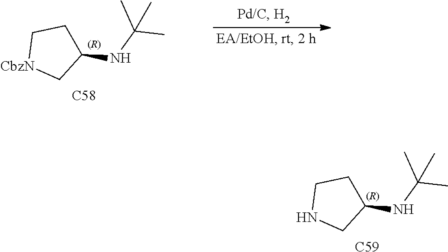

To a solution of benzyl (3R)-3-(tert-butylamino)pyrrolidine-1-carboxylate (2.7 g, 9.769 mmol, 1.00 equiv) in EA (20 mL) and ethanol (30 mL) was added Pd/C (207.93 mg, 1.954 mmol, 0.2 equiv) under nitrogen atmosphere in a 50 mL round-bottom flask. The mixture was hydrogenated at room temperature for 2 hr under hydrogen atmosphere using a hydrogen balloon, filtered through a Celite pad and concentrated under reduced pressure to afford (3R)—N-tert-butylpyrrolidin-3-amine (C59, 590 mg, 42%) as an oil that was taken to the next step without further purification. LCMS (ES, m/z): 143 [M+H]⁺

### Synthesis of Intermediate C60

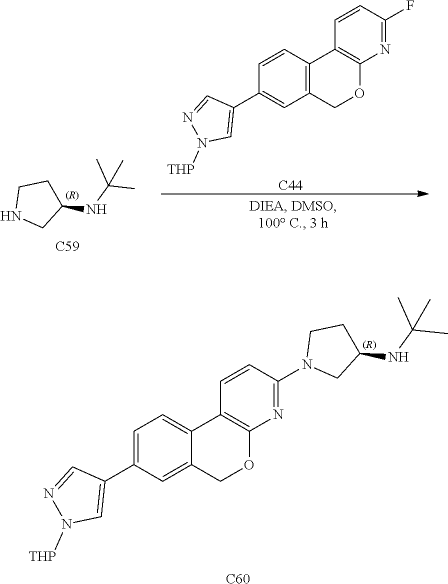

To a stirred solution of 4-{3-fluoro-6H-isochromeno[3,4-b]pyridin-8-yl}-1-(oxan-2-yl)pyrazole (540 mg, 1.537 mmol, 1.00 equiv) and (3R)—N-tert-butylpyrrolidIN-3-amine (655.81 mg, 4.611 mmol, 3 equiv) in DMSO (10 mL) was added DIEA (1986.20 mg, 15.370 mmol, 10 equiv) dropwise at room temperature. The resulting mixture was stirred for 3 hr at 100° C. The mixture was allowed to cool down to room temperature. the reaction was quenched by the addition of water (20 mL) at room temperature. The resulting mixture was extracted with EtOAc (3×5 mL). The Combined organic layers were washed with brine (1×10 mL), dried over anhydrous Na₂SO₄. After filtration, the filtrate was concentrated under vacuum to afford (3R)—N-tert-butyl-1-{8-[1-(oxan-2-yl)pyrazol-4-yl]-6H-isochromeno[3,4-b]pyridin-3-yl}pyrrolidin-3-amine (C60, 700 mg, 96%) as a solid which was used in the next step directly without further purification. LCMS (ES, m/z): 474 [M+H]⁺

### Synthesis of Compound 256

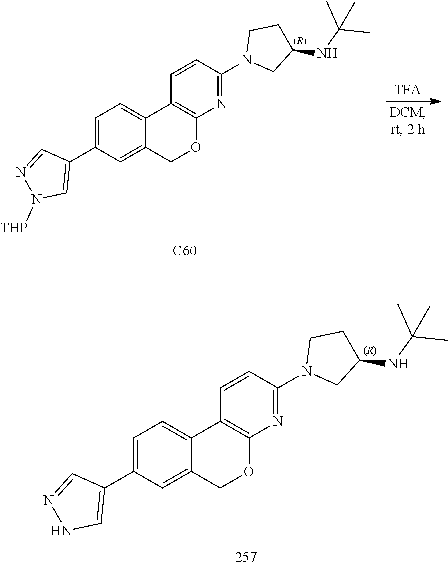

A solution of (3R)—N-tert-butyl-1-{8-[1-(oxan-2-yl)pyrazol-4-yl]-6H-isochromeno[3,4-b]pyridin-3-yl}pyrrOLidin-3-amine (320 mg, 0.676 mmol, 1 equiv) in TFA (5 mL) and DCM (5 mL) was stirred for 2 hr at room temperature. The resulting mixture was concentrated under reduced pressure. The residue was basified to pH 8 with 7 M NH₃(g) in MeOH. The resulting mixture was concentrated under reduced pressure. The residue was purified by Prep-HPLC (Condition 5, Gradient 5) to afford (3R)—N-tert-butyl-1-[8-(1H-pyrazol-4-yl)-6H-isochromeno[3,4-b]pyridin-3-yl]pyrrolidin-3-amine (Compound 256, 204 mg, 77%) as a solid. LCMS (ES, m/z): 390 [M+H]^{+ 1}H NMR (400 MHz, DMSO-d₆) δ 12.93 (s, 1H), 7.97-8.51 (d, J=8.5 Hz, 3H), 7.63-7.52 (m, 2H), 7.45 (d, J=1.7 Hz, 1H), 6.16 (d, J=8.5 Hz, 1H), 5.20 (s, 2H), 3.66 (dd, J=10.3, 6.9 Hz, 1H), 3.55-3.41 (m, 2H), 3.33-3.24 (m, 1H), 2.96 (dd, J=10.2, 7.1 Hz, 1H), 2.19-2.07 (m, 1H), 1.21-1.93 (dq, J=11.9, 8.7 Hz, 2H), 1.08 (s, 9H).

### Example 76: Synthesis of Compound 258

### Synthesis of Intermediate C61

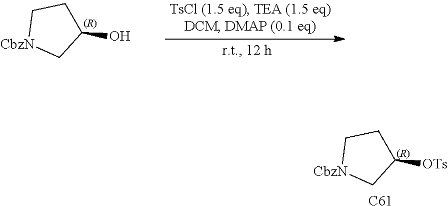

To a stirred solution of benzyl (3R)-3-hydroxypyrrolidine-1-carboxylate (5 g, 22.598 mmol, 1.0 equIV) and TEA (3.66 g, 36.157 mmol, 1.6 equiv) in DCM (40 mL) were added TsC1 (6.89 g, 36.157 mmol, 1.6 equiv) and DMAP (0.28 g, 2.260 mmol, 0.1 equiv) in portions at room temperature. The resulting mixture was stirred for 12 hr at room temperature. The resulting mixture was diluted with H₂O (10 mL). The resulting mixture was extracted with DCM (3×30 mL). The combined organic layers were dried over anhydrous Na₂SO₄. After filtration, the filtrate was concentrated under reduced pressure. The residue was purified by silica gel column chromatography, eluted with PE:EA (1:1) to afford benzyl (3R)-3-[(4-methylbenzenesulfonyl)oxy]pyrrolidine-1-carboxylate (C61, 5.7 g, 67%) as an oil that was taken to the next step without further purification. LCMS (ES, m/z): 376 [M+H]⁺

### Synthesis of Intermediate C62

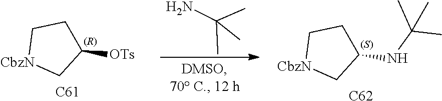

To a sealed tube were added benzyl (3R)-3-[(4-methylbenzenesulfonyl)oxy]pyrrolidine-1-carboxylate (5 g, 13.318 mmol, 1.00 equiv), tertbutylamine (19.48 g, 266.360 mmol, 20 equiv) and DMSO (15 mL). The resulting mixture was stirred for 12 hr at 70° C. The resulting mixture was diluted with H₂O (10 ml). The resulting mixture was extracted with EA (3×15 mL). The combined organic layers were dried over anhydrous Na₂SO₄. After filtration, the filtrate was concentrated under reduced pressure. The residue was purified by silica gel column chromatography, eluted with EA:PE (3:2) to afford benzyl (3S)-3-(tert-butylamino)pyrrolidine-1-carboxylate (C62, 2 g, 54%) as an oil that was taken to the next step without further purification. LCMS (ES, m/z): 277 [M+H]⁺

### Synthesis of Intermediate C63

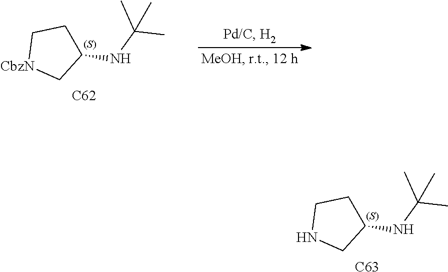

To a solution of benzyl (3S)-3-(tert-butylamino)pyrrolidine-1-carboxylate (1.6 g, 5.789 mmol, 1.00 equiv) in EA (8 mL) and EtOH (12 mL) was added Pd/C (0.49 g, 4.631 mmol, 0.8 equiv) under nitrogen atmosphere. The mixture was hydrogenated at room temperature for 12 hr under hydrogen atmosphere using a hydrogen balloon, filtered through a Celite pad and concentrated under reduced pressure to afford 4-{3-fluoro-6H-isochromeno[3,4-b]pyridin-8-yl}-1-(oxan-2-yl)pyrazole (C63, 400 mg) as an oil which was used in the next step directly without further purification.

### Synthesis of Intermediate C64

A solution of 4-{3-fluoro-6H-isochromeno[3,4-b]pyridin-8-yl}-1-(oxan-2-yl)pyrazole (400 mg, 1.138 mmol, 1.00 equiv), (3S)—N-tert-butylpyrrolidin-3-amine (323.86 mg, 2.276 mmol, 2.0 equiv) And DIEA (441.38 mg, 3.414 mmol, 3.0 equiv) in DMSO (4 mL) was stirred for 3 hr at 100° C. The resulting mixture was diluted with H₂O (10 mL). The aqueous layer was extracted with EA (3×10 mL). The combined organic layers were dried over anhydrous Na₂SO₄. After filtration, the filtrate was concentrated under reduced pressure to afford (3S)—N-tert-butyl-1-{8-[1-(oxan-2-yl)pyrazol-4-yl]-6H-isochromeno[3,4-b]pyridin-3-yl}pyrrolidin-3-amine (C64, 500 mg) as an oil which was used in the next step directly without further purification. LCMS (ES, m/z): 474 [M+H]

### Synthesis of Compound 258

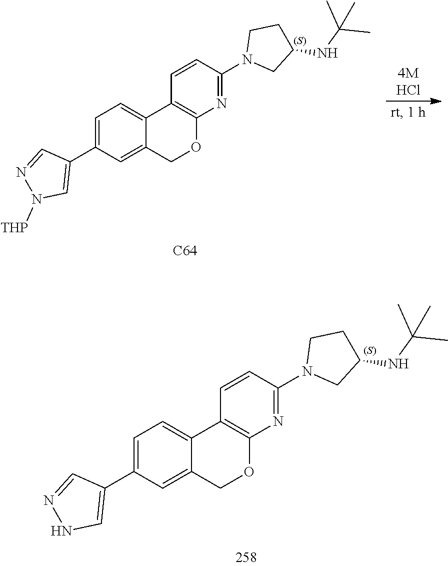

A solution of (3S)—N-tert-butyl-1-{8-[1-(oxan-2-yl)pyrazol-4-yl]-6H-isochromeno[3,4-b]pyridin-3-yl}pyrrolidin-3-amine (500 mg, 1.056 mmol, 1.00 equiv) in HCl (gas) in 1,4-dioxane (8 mL) was stirred for 1 hr at room temperature. The resulting mixture was concentrated under reduced pressure. The residue was purified by reverse flash chromatography with the following conditions: column, C18 silica gel; mobile phase, CH₃CN in water (0.1% NH₃H₂O), 20% to 60% gradient in 15 min; detector, UV 254 nm. This resulted in (3S)—N-tert-butyl-1-[8-(1H-pyrazol-4-yl)-6H-isochromeno[3,4-b]pyridin-3-yl]pyrrolidin-3-amine (Compound 258, 200 mg, 48%) as a solid. LCMS (ES, m/z): 390 [M+H]+¹H NMR (300 MHz, DMSO-d₆) δ 12.93 (s, 1H), 8.17 (s, 1H), 8.02-7.84 (m, 2H), 7.64-7.51 (m, 2H), 7.45 (d, J=1.7 Hz, 1H), 6.17 (d, J=8.5 Hz, 1H), 5.20 (s, 2H), 3.66 (dd, J=10.2, 6.9 Hz, 1H), 3.48 (q, J=9.3, 7.8 Hz, 2H), 3.31-3.17 (m, 1H), 2.95 (dd, J=10.1, 7.2 Hz, 1H), 2.13 (s, 1H), 1.79-1.60 (m, 2H), 1.08 (s, 9H).

### Example 77: Synthesis of Compound 259

### Synthesis of Intermediate C65

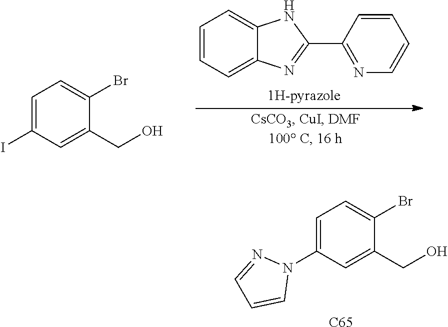 [SMILES: c1ccc(-c2nc3ccccc3[nH]2)nc1 ; OCc1cc(I)ccc1Br ; OCc1cc(-n2cccn2)ccc1Br ; "C65"]

To a stirred solution of (2-bromo-5-iodophenyl)methanol (5 g, 15.978 mmol, 1.00 equiv), 2-(pyridin-2-yl)-1H-1,3-benzodiazole (0.31 g, 1.598 mmol, 0.1 equiv) and pyrazole (1.63 g, 23.967 mmol, 1.5 equiv) in DMF (50 mL) was added Cs₂CO₃ (10.41 g, 31.956 mmol, 2.0 equiv) and CuI (0.30 g, 1.598 mmol, 0.1 equiv) at room temperature under N₂ atmosphere. The resulting mixture was stirred for 16 hr at 100° C. under n₂ atmosphere. The reaction was diluted with water (200 mL) and extracted with EA (150 mL×3). The combined organic layers were washed with water and brine, dried over anhydrous Na₂SO₄. After filtration, the filtrate was concentrated under reduced pressure. The residue was purified by silica gel column chromatography, eluted with PE/EA (2:1) to afford the compound (C65, 1.32 g, 32%) as a solid. LCMS (ES, m/z): 253 [M+H]⁺

### Synthesis of Intermediate C66

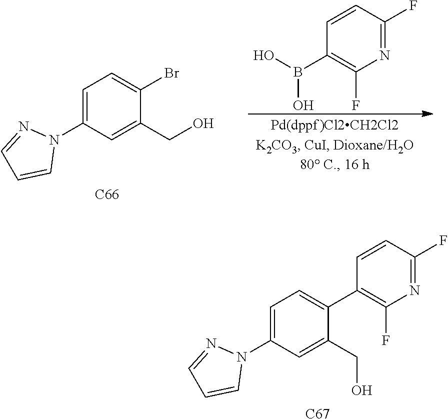 [SMILES: OCc1cc(-n2cccn2)ccc1Br ; OB(O)c1ccc(F)nc1F ; "C66" ; OCc1cc(-n2cccn2)ccc1-c1ccc(F)nc1F ; "C67"]

To a stirred mixture of [2-bromo-5-(pyrazol-1-yl)phenyl]methanol (1.3 g, 5.136 mmol, 1.00 equiv), 2,6-difluoropyridin-3-ylboronic acid (1.63 g, 10.272 mmol, 2.0 equiv) and K₂CO₃ (2.13 g, 15.408 Mmol, 3.0 equiv) in a solution of dioxane/H₂O (5:1, 13 mL) were added CuI (0.10 g, 0.514 mmol, 0.1 equiv) and Pd(dppf)Cl₂·CH₂Cl₂ (0.38 g, 0.514 mmol, 0.1 equiv) at room temperature under N₂ atmosphere. The residue was purified by silica gel column chromatography, eluted with PE/EA (2:1) to afford the title compound (C66, 940 mg, 29%) as a solid. LCMS (ES, m/z): 288 [M+H]⁺

### Synthesis of Intermediate C68

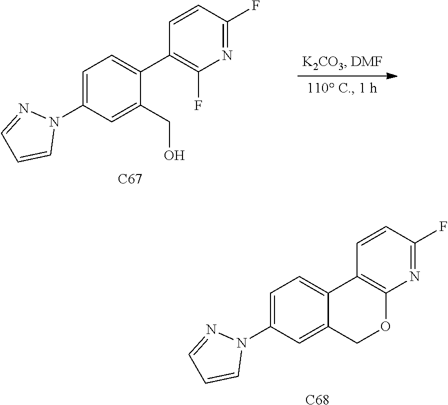 [SMILES: OCc1cc(-n2cccn2)ccc1-c1ccc(F)nc1F ; "C67" ; Fc1ccc2c(n1)OCc1cc(-n3cccn3)ccc1-2 ; "C68"]

To a stirred solution of [2-(2,6-difluoropyridin-3-yl)-5-(pyrazol-1-yl)phenyL]methanol (940 mg, 3.272 mmol, 1.00 equiv) in DMF (8.9 mL) was added K₂CO₃ (1356.70 mg, 9.816 mmol, 3.0 equiv) at room temperature under N₂ atmosphere. The resulting mixture was stirred for 1 hr at 100° C. undeR N₂ atmosphere. The reaction was diluted with water (30 mL) and extracted with EA (30 mL×3). The combined organic layers were washed with water and brine, dried over anhydrous Na₂SO₄ and concentrated under reduced pressure. The residue was purified by silica gel column chromatography, eluted with PE/EA (2:1) to afford 1-{3-fluoro-6H-isochromeno[3,4-b]pyridin-8-yl}pyrazole (C68, 450 mg, 51%) as a solid that was taken to the next step without further purification. LCMS (ES, m/z): 268 [M+H]⁺

### Synthesis of Intermediate C69

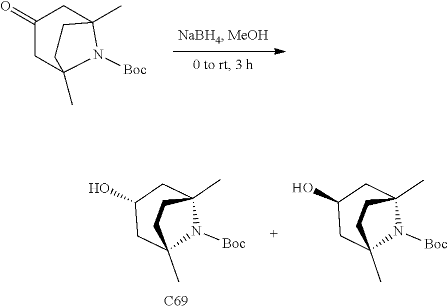

To a stirred mixture of tert-butyl 1,5-dimethyl-3-oxo-8-azabicyclo[3.2.1]octane-8-carboxylate (1 g, 3.947 mmol, 1.00 equiv) in MeOH (10 mL) were added NaBH₄ (0.22 g, 5.921 mmol, 1.5 equiv) in portions at 0° C. The reaction mixture was stirred for 3 hr at ° C. The resulting mixture was concentrated under reduced pressure. The crude product was purified by Prep-HPLC with the following conditions (Column, Xselect CSH OBD Column 30×150 mm Sum; mobile phase, water (0.05% FA) and ACN (10% Phase B up to 50% in 8 min); Detector, UV 220 nm) to afford tert-butyl (1R,3S,5S)-3-hydroxy-1,5-dimethyl-8-azabicyclo[3.2.1]octane-8-carboxylate (C69, 200 mg, 19%) as a solid and tert-butyl (1R,3R,5S)-3-hydroxy-1,5-dimethyl-8-azabicyclo[3.2.1]octane-8-carboxylate (C69, 500 mg, 49%) as a solid that was taken to the next step without further purification. LCMS (ES, m/z): 256 [M+H]⁺

### Synthesis of Intermediate C70

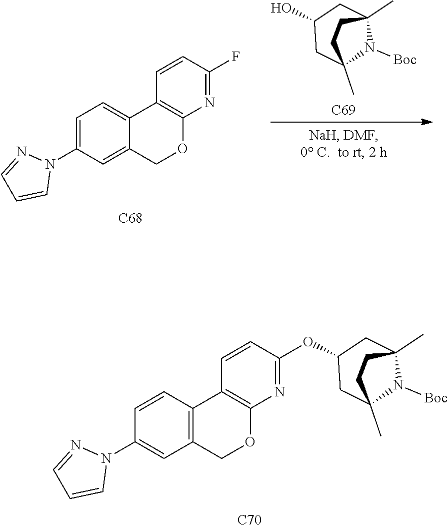

To a solution of tert-butyl (1S,3R,5R)-3-hydroxy-1-methyl-8-azabicyclo[3.2.1]octane-8-CArboxylate (101.58 mg, 0.422 mmol, 1.5 equiv) in DMF (1.5 mL) was added sodium hydride (60% in oil, 22.45 mg) at 0° C. The mixture was stirred for 15 min. 1-{3-fluoro-6H-isochromeno[3,4-b]pyridin-8-yl}pyrazole (75 mg, 0.281 mmol, 1.00 equiv) was added and the mixture was allowed to warm to room temperature and stirred for 2 hr. The reaction was diluted with water (5 mL) and extracted with EA (5 mL×3). The combined organic layers were washed with water and brine, dried over anhydrous Na₂SO₄ and concentrated under reduced pressure. The residue was afford tert-butyl (1R,3S,5S)-1,5-dimethyl-3-{[8-(pyrazol-1-yl)-6H-isochromeno[3,4-b]pyridin-3-yl]oxy}-8-azabicyclo[3.2.1]octane-8-carboxylate (C70, 65 mg, 46%) as a solid that was taken to the next step without further purification. LCMS (ES, m/z): 503 [M+H]⁺

### Synthesis of Compound 259

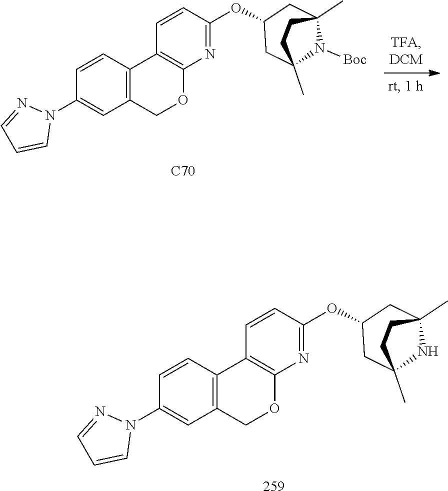

To a stirred solution of tert-butyl (1S,3R,5R)-6,6-difluoro-3-{[8-(1-methyl-6-oxopyridazin-4-yl)-6H-isochromeno[3,4-b]pyridin-3-yl]oxy}-8-azabicyclo[3.2.1]octane-8-carboxylate (65 mg, 0.118 mmol, 1.00 equiv) iN DCM (0.6 mL, 9.438 mmol, 80.23 equiv) was added TFA (0.6 mL, 8.078 mmol, 68.67 equiv) at room temperature. The reaction mixture was stirred for 2 h at room temperature. The resulting mixture was concentrated under reduced pressure. The crude product was purified by Prep-HPLC (Condition 7, Gradient 3) to afford 6,6-difluoro-3-{[8-(6-methoxypyridazin-4-yl)-6H-isochromeno[3,4-b]pyridin-3-yl]oxy}-8-azabicyclo[3.2.1]octane (Compound 259, 19 mg, 35%) as a solid. LCMS (ES, m/z): 403 [M+H]^{+ 1}H NMR (400 MHz, DMSO-d₆) δ 8.52 (d, J=2.5 Hz, 1H), 8.24 (d, J=8.3 Hz, 1H), 7.90-7.81 (m, 2H), 7.78 (dd, J=9.3, 1.9 Hz, 2H), 6.57 (t, J=2.1 Hz, 1H), 6.51 (d, J=8.3 Hz, 1H), 5.39 (s, 2H), 5.23 (tt, J=11.0, 5.8 Hz, 1H), 2.08 (dd, J=12.2, 6.2 Hz, 2H), 1.80 (d, J=8.0 Hz, 2H), 1.55 (d, J=7.9 Hz, 2H), 1.37 (t, J=11.1 Hz, 2H), 1.21 (s, 6H).

### Example 78: Synthesis of Compound 261

### Synthesis of Intermediate C71

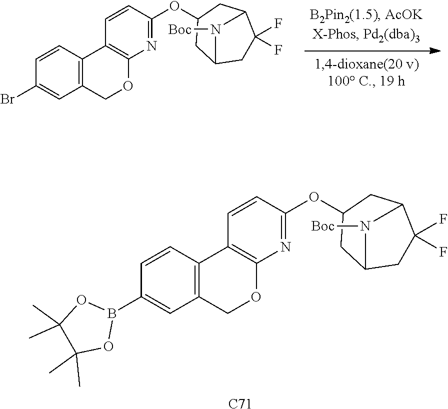 [SMILES: CC(C)(C)OC(=O)N1C2CC(Oc3ccc4c(n3)OCc3cc(Br)ccc3-4)CC1C(F)(F)C2 ; CC(C)(C)OC(=O)N1C2CC(Oc3ccc4c(n3)OCc3cc(B5OC(C)(C)C(C)(C)O5)ccc3-4)CC1C(F)(F)C2 ; "C71"]

To a stirred mixture of tert-butyl-3-({8-bromo-6H-isochromeno[3,4-b]pyridin-3-yl}oxy)-6,6-difluoro-8-azabicyclo[3.2.1]octane-8-carboxylate (100 mg, 0.191 mmol, 1.00 equiv) and bis(pinacolato)diboron (72.78 mg, 0.286 mmol, 1.5 equiv) in 1,4-dioxane (2 mL) was added AcOK (56.26 mg, 0.573 mmol, 3 equiv) and Pd(dppf)Cl₂·CH₂Cl₂ (15.56 mg, 0.019 mmol, 0.1 equiv) at room temperature under nitrogen atmosphere. The resulting mixture was stirred for 19 hr at 100° C. under nitrogen atmosphere. The resulting mixture was used in the next step directly without further purification. LCMS (ES, m/z): 571[M+H]⁺

### Synthesis of Intermediate C72

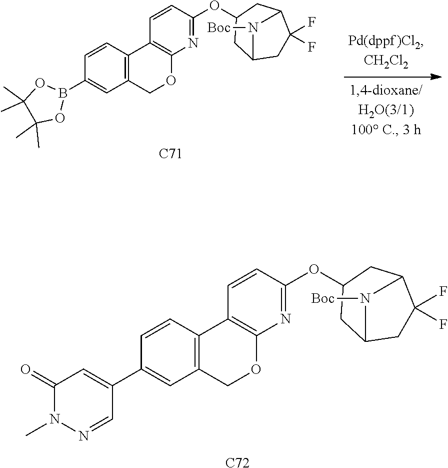 [SMILES: CC(C)(C)OC(=O)N1C2CC(Oc3ccc4c(n3)OCc3cc(B5OC(C)(C)C(C)(C)O5)ccc3-4)CC1C(F)(F)C2 ; "C71" ; Cn1ncc(-c2ccc3c(c2)COc2nc(OC4CC5CC(F)(F)C(C4)N5C(=O)OC(C)(C)C)ccc2-3)cc1=O ; "C72"]

To the stirred mixture of intermediate C71 were added H₂O (0.5 mL), 5-iodo-2-methylpyridazin-3-one (68.27 mg, 0.289 mmol, 1.5 equiv) and Pd(dppf)Cl₂·CH₂Cl₂ (15.71 mg, 0.019 mmol, 0.1 equiv) at room temperature under nitrogen atmosphere. The resulting mixture was stirred for 3 hr at 100° C. under nitrogen atmosphere. The resulting mixture was concentrated under reduced pressure. The residue was purified by silica gel column chromatography, eluted with PE/EA (5:1) to afford tert-butyl-6,6-difluoro-3-{[8-(1-methyl-6-oxopyridazin-4-yl)-6H-isochromeno[3,4-b]pyridin-3-yl]oxy}-8-azabicyclo[3.2.1]octane-8-carboxylate (C72, 90 mg, 84%) as a solid. LCMS (ES, m/z): 553[M+H]⁺

### Synthesis of Compound 261

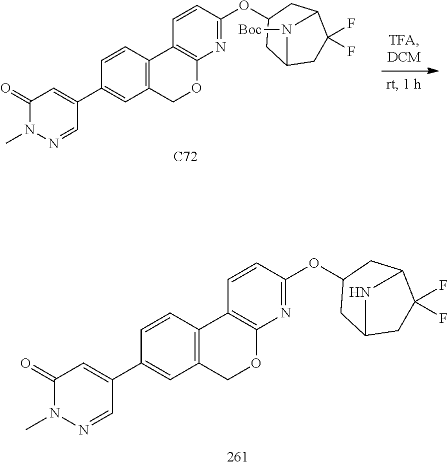 [SMILES: Cn1ncc(-c2ccc3c(c2)COc2nc(OC4CC5CC(F)(F)C(C4)N5C(=O)OC(C)(C)C)ccc2-3)cc1=O ; "C72" ; Cn1ncc(-c2ccc3c(c2)COc2nc(OC4CC5CC(F)(F)C(C4)N5)ccc2-3)cc1=O ; "261"]

To a stirred solution of tert-butyl-6,6-difluoro-3-{[8-(1-methyl-6-oxopyridazin-4-yl)-6H-isochromeno[3,4-b]pyridin-3-yl]oxy}-8-azabicyclo[3.2.1]octane-8-carboxylate (90 mg, 0.163 mmol, 1.00 equiv) in DCM (0.9 mL) was added TFA (0.9 mL, 12.117 mmol, 74.39 equiv) at room temperature. The resulting mixture was stirred for 2 h at room temperature and concentrated under reduced pressure. The residue was purified by Prep-HPLC (Condition 7, Gradient 3) to afford 5-[3-({6,6-difluoro-8-azabicyclo[3.2.1]octan-3-yl}oxy)-6H-isochromeno[3,4-b]pyridin-8-yl]-2-methylpyridazin-3-one (Compound 261, 20.3 mg, 27.55%) as a solid. LCMS (ES, m/z): 453[M+H]+ 1H NMR (300 MHz, DMSO-d6) δ 8.39 (d, J=2.3 Hz, 1H), 8.31 (d, J=8.4 Hz, 1H), 7.88 (d, J=2.0 Hz, 2H), 7.81 (s, 1H), 7.27 (d, J=2.3 Hz, 1H), 6.54 (d, J=8.3 Hz, 1H), 5.38 (s, 2H), 5.37-5.28 (m, 1H), 3.70 (s, 3H), 3.61 (s, 1H), 3.45 (d, J=13.0 Hz, 1H), 2.90 (s, 1H), 2.32 (s, 2H), 2.18 (d, J=11.6 Hz, 1H), 1.63 (dt, J=24.8, 11.4 Hz, 2H).

### Example 79: Synthesis of Compound 262

### Synthesis of Intermediate C73

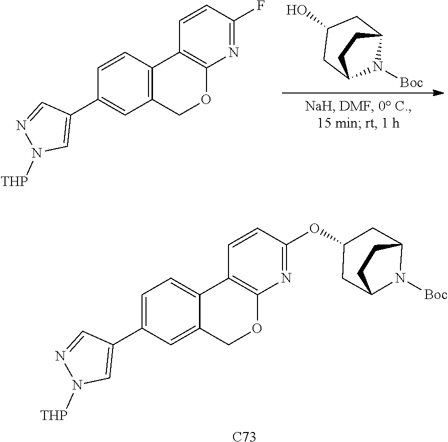

To a stirred solution of tert-butyl (1R,3s,5S)-3-hydroxy-8-azabicyclo [3.2.1] octane-8-carboxylate (58.2 mg, 0.257 mmol, 1.5 equiv) in DMF (1.0 mL) was added NaH (5.7 mg, 0.239 mmol, 1.4 equiv) in portions at 0° C. The resulting mixture was stirred for 15 min at 0° C. To the above mixture was added 3-fluoro-8-(1-(tetrahydro-2H-pyran-2-yl)-1H-pyrazol-4-yl)-6H-isochromeno[3,4-b] pyridine (60.0 mg, 0.171 mmol, 1.0 equiv) in DMF (0.2 mL) dropwise portions over 2 min at 0° C. The resulting mixture was stirred for additional 2 hr at room temperature. The reaction was quenched with water at 0° C. The resulting mixture was extracted with EtOAc (3×3 mL). the combined organic layers were washed with water (3×6 mL), dried over anhydrous Na₂SO₄. After filtration, the filtrate was concentrated under reduced pressure. The residue was purified by silica gel column chromatography, eluted with CH₂Cl₂/MeOH (5:1) to afford tert-butyl(1R,3s,5S)-3-((8-(1-(tetrahydro-2H-pyran-2-yl)-1H-pyrazol-4-yl)-6H-isochromeno[3,4-b]pyridin-3-yl)oxy)-8-azabicyclo[3.2.1]octane-8-carboxylate (C73, 110 mg, 98%) as an oil that was taken to the next step without further purification. LCMS (ES, m/z): 559.4 [M+H]

### Synthesis of Compound 262

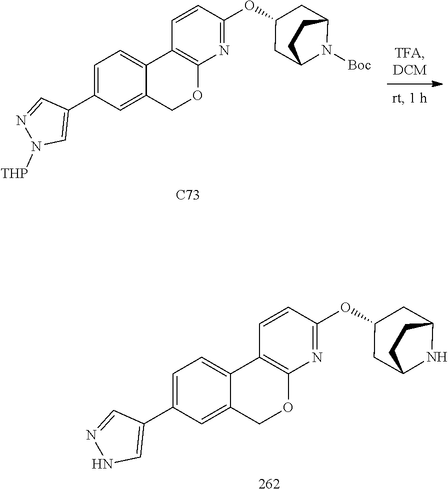

To a stirred solution of tert-butyl (1R,3s,5S)-3-((8-(1-(tetrahydro-2H-pyran-2-yl)-1H-pyrazol-4-yl)-6H-isochromeno[3,4-b]pyridin-3-yl)oxy)-8-azabicyclo[3.2.1]octane-8-carboxylate (110 mg, 0.197 mmol, 1.0 equiv) in DCM (1.1 mL) was added TFA (0.55 mL) dropwise portions at room temperature. The resulting mixture was stirred for 1 hr at room temperature. The resulting mixture was concentrated under reduced pressure. The residue was neutralized to pH 7 with saturated Na₂CO₃ (aq.). The crude product was purified by Prep-HPLC (Condition 5, Gradient 4) to afford 3-(((1R,3s,5S)-8-azabicyclo [3.2.1] octan-3-yl) oxy)-8-(1H-pyrazol-4-yl)-6H-isochromeno[3,4-b]pyridine (Compound 262, 30.4 mg, 41%) as a solid. LCMS (ES, m/z): 375.2 [M+H]+ 1H NMR (300 MHz, DMSO-d₆) δ 12.97 (s, 1H), 8.18 (d, J=8.4 Hz, 1H), 8.13-8.03 (m, 2H), 7.72 (d, J=8.2 Hz, 1H), 7.61 (dd, J=8.1, 1.8 Hz, 1H), 7.52 (d, J=1.7 Hz, 1H), 6.47 (d, J=8.3 Hz, 1H), 5.32 (s, 2H), 5.25-5.14 (m, 1H), 3.49-3.44 (m, 2H), 2.22-2.10 (m, 1H), 2.08-1.97 (m, 2H), 1.77-1.60 (m, 4H), 1.55-1.45 (m, 2H).

### Example 80: Synthesis of Compound 265

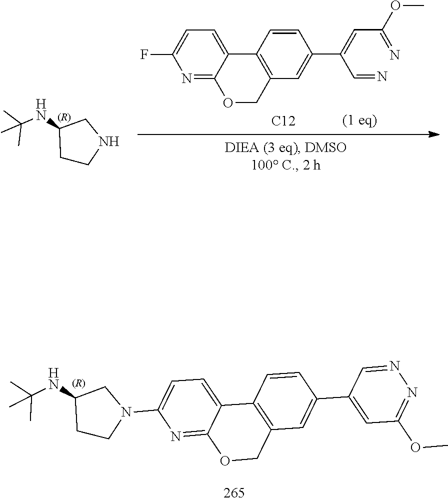

A mixture of (3R)—N-tert-butylpyrrolidin-3-amine (32.19 mg, 0.226 mmol, 1 equiv), DIEA (87.75 mg, 0.678 mmol, 3 equiv), and DMSO (1 mL) at room temperature was stirred for 2 h at 100° C. The reaction mixture was quenched with water at 0° C. A solid precipitated that was collected by filtration, then purified by Prep-HPLC (Condition 10, Gradient 1) to afford (3R)—N-tert-butyl-1-[8-(6-methoxypyridazin-4-yl)-6H-isochromeno[3,4-b]376yrrolid-3-yl]376yrrolidine-3-amine (18.1 mg, 19%) as a solid. LCMS (ES, m/z): 431 [M+H]⁺. ¹H NMR (400 MHz, DMSO-d₆) δ 9.32 (d, J=1.9 Hz, 1H), 8.06 (d, J=8.5 Hz, 1H), 7.88 (dd, J=8.2, 2.0 Hz, 1H), 7.80 (d, J 2.0 Hz, 1H), 7.76 (d, J=8.2 Hz, 1H), 7.48 (d, J=1.9 Hz, 1H), 6.21 (d, J=8.5 Hz, 1H), 5.26 (s, 2H), 4.08 (s, 3H), 3.68 (t, J=8.7 Hz, 1H), 3.55-3.45 (m, 2H), 3.35-3.28 (m, 1H), 2.97 (s, 1H), 2.13 (s, 1H), 1.70 (s, 1H), 1.08 (s, 9H).

### Example 81: Synthesis of Compound 271

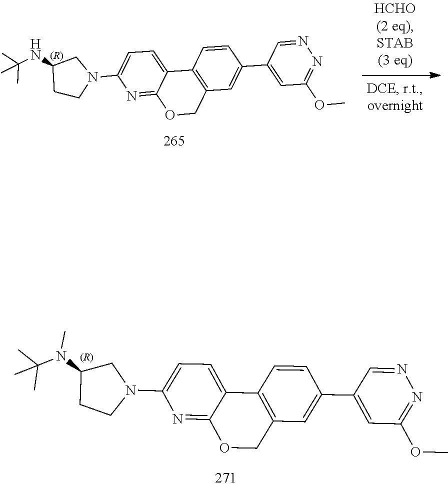

(3R)—N-tert-butyl-1-[8-(6-methoxypyridazin-4-yl)-6H-isochromeno [3,4-b]376yrrolid-3-yl]376yrrolidine-3-amine (60 mg, 0.139 mmol, 1 equiv), HCHO (8.35 mg, 0.278 mmol, 2 equiv), and DCE (1 mL) were combined at room temperature. To the reaction mixture was added STAB (88.40 mg, 0.417 mmol, 3 equiv) in portions over 2 min at 0° C. The resulting mixture was stirred overnight at room temperature, then quenched with water at 0° C. The resulting mixture was extracted with DCM (5 mL×3) and concentrated under reduced pressure to give a residue. The residue was purified by Prep-HPLC (Condition 11, Gradient 1) to afford (3R)—N-tert-butyl-1-[8-(6-methoxypyridazin-4-yl)-6H-isochromeno [3,4-b]377yrrolid-3-yl]-N-methylpyrrolidin-3-amine (6.3 mg, 10%) as a solid. LCMS (ES, m/z): 445 [M+H]⁺. ¹H NMR (400 MHz, DMSO-d₆) δ 9.33 (d, J=1.9 Hz, 1H), 8.08 (d, J=8.6 Hz, 1H), 7.89 (dd, J=8.2, 2.0 Hz, 1H), 7.84 (m, 1H), 7.74 (m, 1H), 7.50 (d, J=2.0 Hz, 1H), 6.25 (d, J=8.5 Hz, 1H), 5.27 (s, 2H), 4.08 (s, 3H), 3.95-3.84 (m, 1H), 3.57 (t, J=10.1 Hz, 1H), 3.30 (s, 1H), 3.23-3.14 (m, 2H), 2.19 (s, 3H), 1.98 (q, J=10.1, 9.7 Hz, 1H), 1.87 (dd, J=11.8, 6.4 Hz, 1H), 1.24 (s, OH), 1.09 (s, 9H).

### Example 82: Synthesis of Compound 264

### Synthesis of Intermediate C74

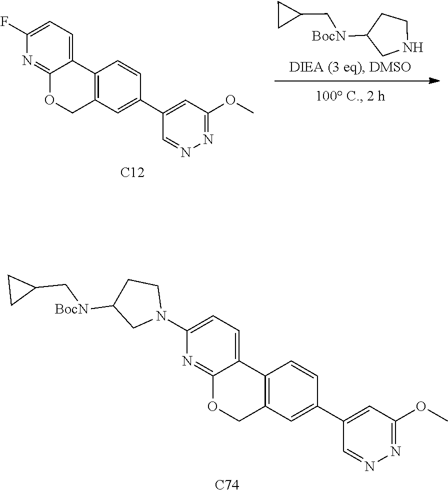 [SMILES: COc1cc(-c2ccc3c(c2)COc2nc(F)ccc2-3)cnn1 ; CC(C)(C)OC(=O)N(CC1CC1)C1CCNC1 ; "C12" ; COc1cc(-c2ccc3c(c2)COc2nc(N4CCC(N(CC5CC5)C(=O)OC(C)(C)C)C4)ccc2-3)cnn1 ; "C74"]

A mixture of 5-{3-fluoro-6H-isochromeno[3,4-b]377yrrolid-8-yl}-3-methoxypyridazine (70 mg, 0.226 mmol, 1 equiv), DIEA (87.75 mg, 0.678 mmol, 3 equiv) and DMSO (5 mL) was stirred for 2 h at 100° C., then quenched with water at 0° C. A solid precipitated that was collected by filtration to afford tert-butyl N-(cyclopropylmethyl)-N-{1-[8-(6-methoxypyridazin-4-yl)-6H-isochromeno[3,4-b]377yrrolid-3-yl]377yrrolidine-3-yl}carbamate (80 mg, 64%) as a solid.

### Synthesis of Compound 264

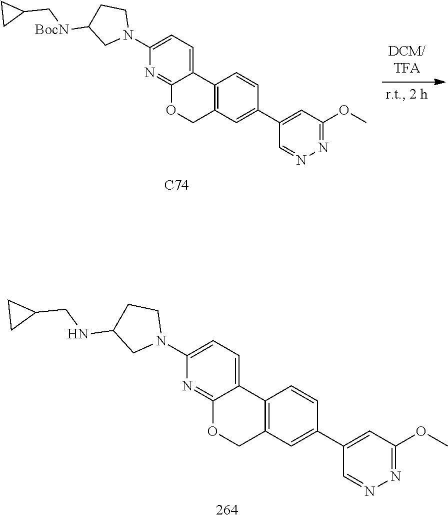 [SMILES: COc1cc(-c2ccc3c(c2)COc2nc(N4CCC(N(CC5CC5)C(=O)OC(C)(C)C)C4)ccc2-3)cnn1 ; "C74" ; COc1cc(-c2ccc3c(c2)COc2nc(N4CCC(NCC5CC5)C4)ccc2-3)cnn1 ; "264"]

A mixture of tert-butyl N-(cyclopropylmethyl)-N-{1-[8-(6-methoxypyridazin-4-yl)-6H-isochromeno[3,4-b]377yrrolid-3-yl]377yrrolidine-3-yl}carbamate (80 mg, 0.151 mmol, 1.00 equiv), DCM (2.86 mL), and TFA (0.57 mL) was stirred for 2 h at room temperature. The resulting mixture was concentrated under reduced pressure to give a residue. The residue was purified by Prep-HPLC (Condition 10, Gradient 2) to afford N-(cyclopropylmethyl)-1-[8-(6-methoxypyridazin-4-yl)-6H-isochromeno [3,4-b]378yrrolid-3-yl]378yrrolidine-3-amine (13.4 mg, 20%) as a solid. LCMS (ES, m/z): 429 [M+H]⁺. ¹H NMR (400 MHz, DMSO-d₆) δ 9.33 (d, J=1.9 Hz, 1H), 8.07 (d, J=8.6 Hz, 1H), 7.89 (dd, J=8.2, 2.0 Hz, 1H), 7.81 (d, J=1.9 Hz, 1H), 7.77 (d, J=8.3 Hz, 1H), 7.50 (d, J=1.9 Hz, 1H), 6.23 (d, J=8.5 Hz, 1H), 5.27 (s, 2H), 4.08 (s, 3H), 3.57 (dd, J=10.7, 6.0 Hz, 1H), 3.50 (dd, J=11.6, 5.3 Hz, 1H), 3.44-3.35 (m, 2H), 3.19 (dd, J=10.8, 4.8 Hz, 1H), 2.43 (d, J=6.7 Hz, 2H), 2.08 (dq, J=12.9, 6.5 Hz, 1H), 1.80 (dq, J=12.9, 6.6 Hz, 1H), 0.93-0.82 (m, 1H), 0.45-0.36 (m, 2H), 0.16-0.08 (m, 2H).

### Example 83: Synthesis of Compounds 273, 274, and 314

### Synthesis of Intermediate C75

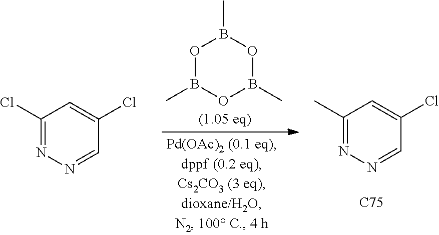

A mixture of 3,5-dichloropyridazine (1 g, 6.713 mmol, 1 equiv), bis(2,4,6-trimethyl-1,3,5,2,4,6-trioxatriborinane) (1.77 g, 7.049 mmol, 1.05 equiv), Dppf (0.74 g, 1.343 mmol, 0.2 equiv), Pd(Oac)₂ (0.15 g, 0.671 mmol, 0.1 equiv), and Cs₂CO₃ (1.53 g, 20.139 mmol, 3 equiv) in dioxane (8 mL) and water (2 mL) was stirred for 4 h at 100° C. under nitrogen atmosphere, then cooled to room temperature. The resulting mixture was extracted with ethyl acetate (1×100 mL), the organic layer dried over anhydrous Na₂SO₄, and filtered. After filtration, the filtrate was concentrated under reduced pressure to give a residue. The residue was purified by silica gel column chromatography, eluted with PE/EA (5:1) to afford 5-chloro-3-methylpyridazine (86 mg, 10%) as a solid.

### Synthesis of Compound 314

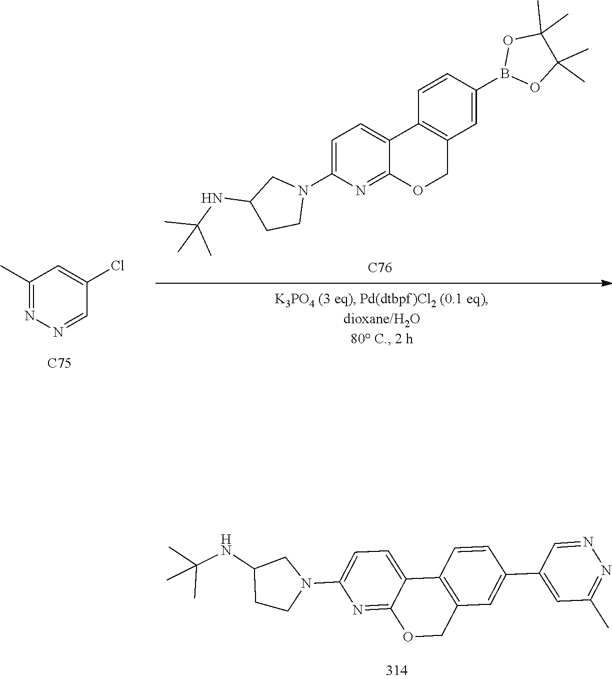

A mixture of 5-chloro-3-methylpyridazine (81 mg, 0.630 mmol, 1 equiv), N-tert-butyl-l-[8-(4,4,5,5-tetramethyl-1,3,2-dioxaborolan-2-yl)-6H-isochromeno[3,4-b]379yrrolid-3-yl]379yrrolidine-3-amine (283.15 mg, 0.630 mmol, 1 equiv), and K₃PO₄ (401.22 mg, 1.890 mmol, 3 equiv) in dioxane (0.65 mL) and water (0.16 mL) was stirred for 2 h at 80° C. under nitrogen atmosphere, then cooled to room temperature. The resulting mixture was extracted with ethyl acetate (1×50 mL), the organic layer was dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford N-tert-butyl-1-[8-(6-methylpyridazin-4-yl)-6H-isochromeno[3,4-b]379yrrolid-3-yl]379yrrolidine-3-amine (207 mg, 79%) as a solid.

### Synthesis of Compounds 273 and 274

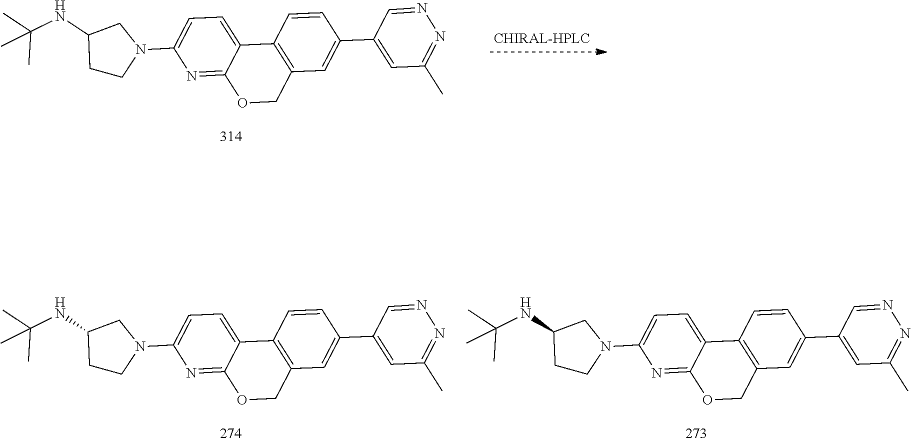

N-tert-butyl-1-[8-(6-methylpyridazin-4-yl)-6H-isochromeno[3,4-b]379yrrolid-3-yl]379yrrolidine-3-amine was purified by chiral Prep-HPLC (Condition 1, Gradient 1) to afford Compound 273 (48.6 mg, 24%) and Compound 274 (47.4 mg, 23%) as solids. Compound 273: LCMS (ES, m/z): 416[M+H]⁺. ¹H NMR (400 MHz, DMSO-d₆) δ 9.47 (d, J=2.3 Hz, 1H), 8.07 (d, J=8.6 Hz, 1H), 7.96-7.84 (m, 2H), 7.84-7.75 (m, 2H), 6.21 (d, J=8.5 Hz, 1H), 5.27 (s, 2H), 3.72-3.63 (m, 1H), 3.55-3.44 (m, 2H), 3.28 (s, 1H), 2.96 (t, J=8.8 Hz, 1H), 2.67 (s, 3H), 2.15-2.09 (m, 1H), 1.70 (s, 2H), 1.07 (s, 9H). Compound 274: LCMS (ES, m/z): 416[M+H]⁺. ¹H NMR (400 MHz, DMSO-d₆) δ 9.47 (d, J=2.3 Hz, 1H), 8.07 (d, J=8.5 Hz, 1H), 7.89 (dd, J 6.3, 2.2 Hz, 2H), 7.84-7.75 (m, 2H), 6.21 (d, J=8.5 Hz, 1H), 5.27 (s, 2H), 3.67 (t, J=8.7 Hz, 1H), 3.50 (dt, J=21.3, 8.6 Hz, 2H), 3.28 (s, 1H), 2.97 (dd, J=10.3, 7.1 Hz, 1H), 2.67 (s, 3H), 2.13 (dd, J=10.9, 5.0 Hz, 1H), 1.77-1.63 (m, 2H), 1.07 (s, 9H).

### Example 84: Synthesis of Compound 284

### Synthesis of Intermediate C77

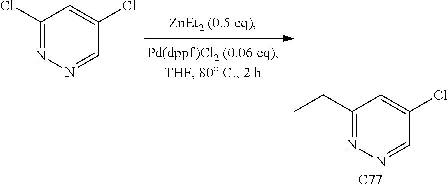

A mixture of 3,5-dichloropyridazine (500 mg, 3.356 mmol, 1 equiv), ZnEt₂ (207 mg, 1.678 mmol, 0.5 equiv), and Pd(dppf)Cl₂ (147 mg, 0.201 mmol, 0.06 equiv) in THF (5 mL) was stirred for 2 h at 80° C. under nitrogen atmosphere, then cooled to room temperature. The resulting mixture was poured into NH₄Cl (aq) and extracted with ethyl acetate (1×50 mL). The organic layer was dried over anhydrous Na₂SO₄, and filtered. After filtration, the filtrate was concentrated under reduced pressure to give a residue. The residue was purified by silica gel column chromatography, eluted with PE/EA (5:1) to afford 5-chloro-3-ethylpyridazine (85 mg, 18%) as a solid.

### Synthesis of Compound 284

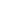

A mixture of 5-chloro-3-ethylpyridazine (85 mg, 0.596 mmol, 1 equiv), N-tert-butyl-1-[8-(4,4,5,5-tetramethyl-1,3,2-dioxaborolan-2-yl)-6H-isochromeno[3,4-b]pyridin-3-yl]pyrrolidin-3-amine (268 mg, 0.596 mmol, 1 equiv), K₃PO₄ (380 mg, 1.788 mmol, 3 equiv), and Pd(DtBPF)Cl₂ (39 mg, 0.060 mmol, 0.1 equiv) in 1,4-dioxane (0.68 mL) and water (0.17 mL) was stirred for 2 h at 80° C. under nitrogen atmosphere, then cooled to room temperature. The resulting mixture was extracted with ethyl acetate (1×10 mL). The organic layer was dried over anhydrous Na₂SO₄ and filtered. After filtration, the filtrate was concentrated under reduced pressure to give a residue. The residue was purified by silica gel column chromatography, eluted with CH₂Cl₂/MeOH (10:1), followed by Prep-HPLC (Condition 10, Gradient 3) to afford N-tert-butyl-1-[8-(6-ethylpyridazin-4-yl)-6H-isochromeno[3,4-b]pyridin-3-yl]pyrrolidin-3-amine (29.5 mg, 11%) as a solid. LCMS (ES, m/z): 430 [M+H]⁺. ¹H NMR (400 MHz, DMSO-d₆) δ 9.49 (d, J=2.3 Hz, 1H), 8.07 (d, J=8.6 Hz, 1H), 7.90 (q, J=2.9, 2.4 Hz, 2H), 7.83 (d, J=1.9 Hz, 1H), 7.79 (d, J=8.3 Hz, 1H), 6.21 (d, J=8.5 Hz, 1H), 5.28 (s, 2H), 3.67 (t, J=8.7 Hz, 1H), 3.48 (dd, J=21.8, 8.8 Hz, 2H), 3.34 (s, 1H), 2.98 (q, J=7.6 Hz, 3H), 2.12 (d, J=10.1 Hz, 1H), 1.70 (p, J=9.2 Hz, 2H), 1.34 (t, J=7.6 Hz, 3H), 1.07 (s, 9H).

### Example 85: Synthesis of Compound 263

### Synthesis of Intermediate C78

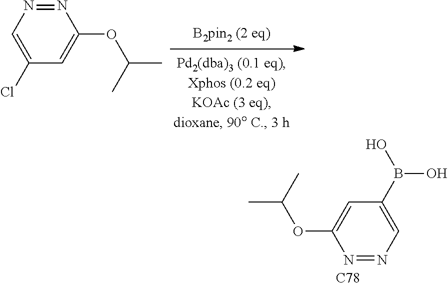

A mixture of 5-chloro-3-isopropoxypyridazine (200 mg, 1.159 mmol, 1 equiv), KOAc (341 mg, 3.477 mmol, 3 equiv), 4,4,5,5-tetramethyl-2-(tetramethyl-1,3,2-dioxaborolan-2-yl)-1,3,2-dioxaborolane (588 mg, 2.318 mmol, 2 equiv), Pd₂(dba)₃ (94 mg, 0.116 mmol, 0.1 equiv), and XPhos (111 mg, 0.232 mmol, 0.2 equiv) in 1,4-dioxane (6 mL) was stirred for 3 h at 90° C. under nitrogen atmosphere. The resulting mixture was filtered, and the filter cake was washed with 1,4-dioxane (1×10 mL). The filtrate was concentrated under reduced pressure to afford 6-isopropoxypyridazin-4-ylboronic acid (150 mg, 71%) as an oil.

### Synthesis of Compound 263

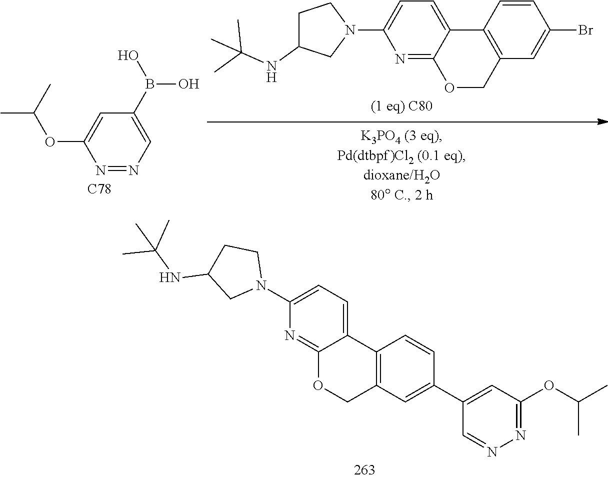

A mixture of 1-{8-bromo-6H-isochromeno[3,4-b]pyridin-3-yl}-N-tert-butylpyrrolidin-3-amine (100 mg, 0.249 mmol, 1 equiv), 6-isopropoxypyridazin-4-ylboronic acid (113 mg, 0.623 mmol, 2.5 equiv), Pd(DtBPF)Cl₂ (16 mg, 0.025 mmol, 0.1 equiv), and K₃PO₄ (158 mg, 0.747 mmol, 3 equiv) in 1,4-dioxane (0.8 mL) and water (0.2 mL) was stirred for 2 h at 80° C. under nitrogen atmosphere. The resulting mixture was extracted with ethyl acetate (1×10 mL). The organic layer was dried over anhydrous Na₂SO₄ and filtered. After filtration, the filtrate was concentrated under reduced pressure to give a residue. The residue was purified by silica gel column chromatography, eluted with CH₂Cl₂/MeOH (10:1), followed by Prep-HPLC (Condition 11, Gradient 2) to afford N-tert-butyl-1-[8-(6-isopropoxypyridazin-4-yl)-6H-isochromeno[3,4-b]pyridin-3-yl]pyrrolidin-3-amine (18.9 mg, 17%) as a solid. LCMS (ES, m/z): 460 [M+H]⁺. ¹H NMR (400 MHz, DMSO-d₆) δ 9.28 (d, J=2.0 Hz, 1H), 8.07 (d, J=8.4 Hz, 1H), 7.89 (dd, J 8.2, 2.0 Hz, 1H), 7.82 (d, J=1.9 Hz, 1H), 7.76 (d, J=8.2 Hz, 1H), 7.42 (d, J=1.9 Hz, 1H), 6.21 (d, J=8.4 Hz, 1H), 5.50 (hept, J=6.2 Hz, 1H), 5.26 (s, 2H), 3.68 (s, 1H), 3.51 (d, J=9.5 Hz, 2H), 3.29 (s, 1H), 2.97 (s, 1H), 2.13 (s, 1H), 1.71 (s, 1H), 1.39 (d, J=6.2 Hz, 6H), 1.08 (s, 9H).

### Example 86: Synthesis of Compounds 286, 292, and 293

### Synthesis of Compound 293

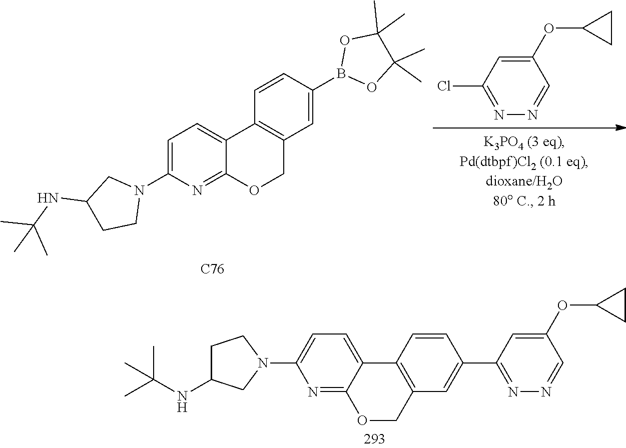

A mixture of N-tert-butyl-1-[8-(4,4,5,5-tetramethyl-1,3,2-dioxaborolan-2-yl)-6H-isochromeno[3,4-b]pyridin-3-yl]pyrrolidin-3-amine (178.17 mg, 0.396 mmol, 1 equiv), 5-methoxypyridazin-3-ol (50 mg, 0.396 mmol, 1 equiv), Pd(dtbpf)Cl₂ (25.84 mg, 0.040 mmol, 0.1 equiv), dioxane (5 mL, 59.020 mmol, 148.87 equiv), and K₃PO₄ (252.47 mg, 1.188 mmol, 3 equiv) in H₂O (1 mL, 55.509 mmol, 140.01 equiv) was stirred for 2 h at 80° C. under nitrogen atmosphere, then cooled to room temperature. The resulting mixture was quenched by the addition of water (5 mL) at room temperature and extracted with CH₂Cl₂ (3×5 mL). The organic layers were combined, washed with brine (1×5 mL), dried over anhydrous Na₂SO₄, and filtered. After filtration, the filtrate was concentrated under reduced pressure to give a residue. The residue was purified by Prep-TLC (CH₂Cl₂/MeOH 10:1), followed by Prep-HPLC (Condition 11, Gradient 3) to afford N-tert-butyl-1-[8-(5-methoxypyridazin-3-yl)-6H-isochromeno[3,4-b]pyridin-3-yl]pyrrolidin-3-amine (14.9 mg, 90%) as a solid.

| Compound Structure | Analysis data |
|---|---|
| 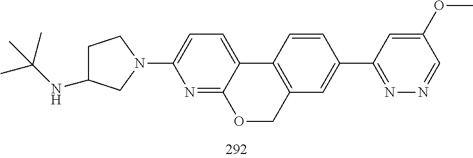 [SMILES: COc1cnnc(-c2ccc3c(c2)COc2nc(N4CCC(NC(C)(C)C)C4)ccc2-3)c1] | LCMS (ES, m/z): 432 + H]⁺. ¹H NMR (400 MHz, DMSO-d₆) δ 8.93 (d, J = 2.8 Hz, 1H), 8.15 (dd, J = 8.2, 2.0 Hz, 1H), 8.10 − 8.04 (m, 2H), 7.78 (d, J = 8.3 Hz, 1H), 7.71 (d, J = 2.8 Hz, 1H), 6.22 (d, J = 8.5 Hz, 1H), 5.30 (s, 2H), 4.01 (s, 3H), 3.68 (t, J = 8.8 Hz, 1H), 3.51 (dd, J = 20.3, 10.4 Hz, 2H), 3.34 (t, J = 4.2 Hz, 1H), 2.97 (t, J = 8.8 Hz, 1H), 2.13 (s, 1H), 1.70 (s, 2H), 1.08 (s, 9H). |
| 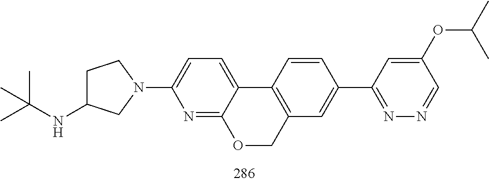 [SMILES: CC(C)Oc1cnnc(-c2ccc3c(c2)COc2nc(N4CCC(NC(C)(C)C)C4)ccc2-3)c1] | LCMS (ES, m/z): 460 [M + H]⁺. ¹H NMR (400 MHz, DMSO-d₆) δ 8.88 (d, J = 2.7 Hz, 1H), 8.13 (dd, J = 8.2, 1.9 Hz, 1H), 8.10 − 8.02 (m, 2H), 7.76 (d, J = 8.2 Hz, 1H), 7.68 (d, J = 2.8 Hz, 1H), 6.21 (d, J = 8.5 Hz, 1H), 5.30 (s, 2H), 5.05 (h, J = 6.0 Hz, 1H), 3.72 − 3.63 (m, 1H), 3.50 (ddd, J = 21.8, 13.5, 5.8 Hz, 2H), 3.37 − 3.26 (m, 1H), 2.96 (dd, J = 10.3, 7.2 Hz, 1H), 2.14 (dd, J = 11.6, 6.5 Hz, 1H), 1.77 − 1.65 (m, 2H), 1.36 (d, J = 6.0 Hz, 6H), 1.07 (s, 9H). |
| 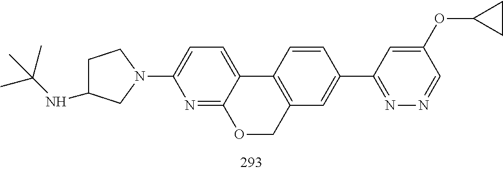 [SMILES: CC(C)(C)NC1CCN(c2ccc3c(n2)OCc2cc(-c4cc(OC5CC5)cnn4)ccc2-3)C1] | LCMS (ES, m/z): 458 [M + H]⁺. ¹H NMR (400 MHz, DMSO-d₆) δ 8.99 (d, J = 2.7 Hz, 1H), 8.13 (dd, J = 8.2, 1.9 Hz, 1H), 8.10 − 8.02 (m, 2H), 7.83 (d, J = 2.8 Hz, 1H), 7.78 (d, J = 8.2 Hz, 1H), 6.22 (d, J = 8.5 Hz, 1H), 5.31 (s, 2H), 4.23 (dq, J = 6.1, 3.0 Hz, 1H), 3.73 − 3.64 (m, 1H), 3.50 (s, 2H), 2.99 (d, J = 9.6 Hz, 1H), 2.13 (dd, J = 11.8, 6.1 Hz, 1H), 1.76 − 1.66 (m, 2H), 1.08 (s, 9H), 0.93 (t, J = 6.3 Hz, 2H), 0.80 (s, 2H). |

### Example 87: Synthesis of Compounds 282, 283, 287, 288, 296, and 297

### Synthesis of Intermediate C79

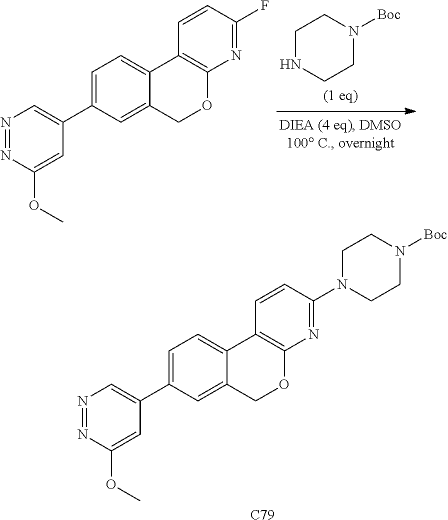

A solution of 5-{3-fluoro-6H-isochromeno[3,4-b] pyridin-8-yl}-3-methoxypyridazine (100 mg, 0.323 mmol, 1.0 equiv) in DMSO (1 mL) was treated with tert-butyl piperazine-1-carboxylate (60.22 mg, 0.323 mmol, 1.0 equiv) followed by DIEA (167.15 mg, 1.292 mmol, 4.0 equiv) at room temperature. The resulting mixture was stirred overnight at 100° C. under nitrogen atmosphere, then extracted with ethyl acetate (3×50 mL). The organic layers were combined, washed with brine (3×30 mL), dried over anhydrous Na₂SO₄, and filtered. After filtration, the filtrate was concentrated under reduced pressure to afford tert-butyl 4-[8-(6-methoxypyridazin-4-yl)-6H-isochromeno[3,4-b]pyridin-3-yl]piperazine-1-carboxylate (100 mg) as a solid.

### Synthesis of Compound 282

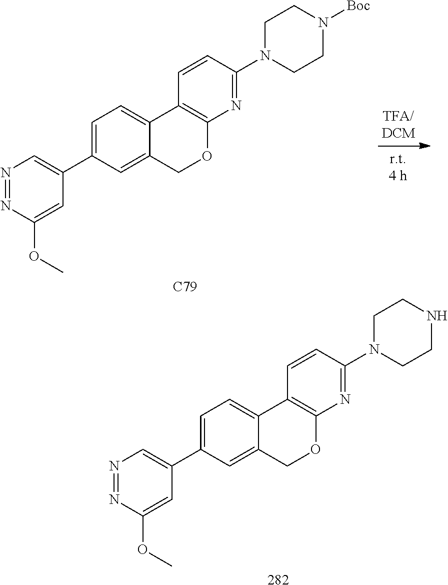 [SMILES: COc1cc(-c2ccc3c(c2)COc2nc(N4CCN(C(=O)OC(C)(C)C)CC4)ccc2-3)cnn1 ; "C79" ; COc1cc(-c2ccc3c(c2)COc2nc(N4CCNCC4)ccc2-3)cnn1 ; "282"]

A solution of tert-butyl 4-[8-(6-methoxypyridazin-4-yl)-6H-isochromeno[3,4-b] pyridin-3-yl]piperazine-1-carboxylate (80 mg, 0.168 mmol, 1.0 equiv) in DCM (1 mL) was treated with TFA (0.1 mL). The reaction mixture was stirred for 4 h at room temperature under nitrogen atmosphere, then basified to pH 6 with saturated Na₂CO₃ (aq.). The resulting mixture was extracted with CH₂Cl₂ (3×100 mL). The organic layers were combined, washed with brine (3×100 mL), dried over anhydrous Na₂SO₄, and filtered. After filtration, the filtrate was concentrated under reduced pressure to give a residue. The residue was purified by Prep-HPLC (Condition 12, Gradient 1) to afford 3-methoxy-5-[3-(piperazin-1-yl)-6H-isochromeno[3,4-b]pyridin-8-yl] pyridazine (11.9 mg, 19%) as a solid.

Compound Structure
Analysis data

| | |
|---|---|
| 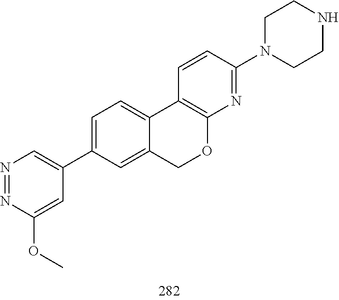 [SMILES: COc1cc(-c2ccc3c(c2)COc2nc(N4CCNCC4)ccc2-3)cnn1] | LCMS (ES, m/z): 376 [M + H]⁺. ¹H NMR (400 MHz, DMSO-d₆) δ 9.33 (d, J = 1.9 Hz, 1H), 8.10 (d, J = 8.7 Hz, 1H), 7.90 (dd, J = 8.1, 2.0 Hz, 1H), 7.86 − 7.75 (m, 2H), 7.51 (d, J = 1.9 Hz, 1H), 6.57 (d, J = 8.6 Hz, 1H), 5.29 (s, 2H), 4.08 (s, 3H), 3.46 (t, J = 5.1 Hz, 4H), 2.76 (dd, J = 6.2, 3.8 Hz, 4H). |
| 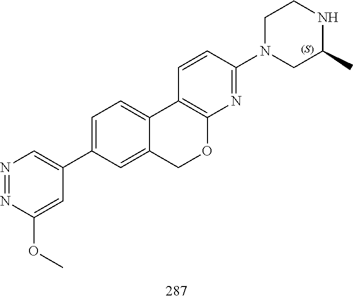 | LCMS (ES, m/z): 390 [M + H]⁺. ¹H NMR (400 MHz, DMSO-d₆) δ 9.33 (d, J = 1.9 Hz, 1H), 8.10 (d, J = 8.7 Hz, 1H), 7.90 (dd, J = 8.2, 2.0 Hz, 1H), 7.86 − 7.76 (m, 2H), 7.51 (d, J = 2.0 Hz, 1H), 6.58 (d, J = 8.7 Hz, 1H), 5.29 (s, 2H), 4.20 − 4.09 (m, 2H), 4.08 (s, 3H), 2.93 (dt, J = 11.5, 2.5 Hz, 1H), 2.75 (td, J = 11.9, 2.9 Hz, 1H), 2.70 − 2.60 (m, 2H), 2.40 − 2.31 (m, 1H), 1.02 (d, J = 6.3 Hz, 3H). |
| 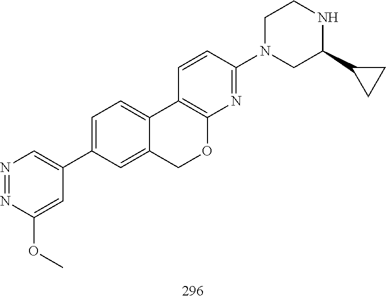 [SMILES: COc1cc(-c2ccc3c(c2)COc2nc(N4CCN[C@@H](C5CC5)C4)ccc2-3)cnn1] | LCMS (ES, m/z): 416 [M + H]⁺. ¹H NMR (400 MHz, DMSO-d₆) δ 9.33 (d, J = 1.9 Hz, 1H), 8.10 (d, J = 8.6 Hz, 1H), 7.90 (dd, J = 8.2, 2.0 Hz, 1H), 7.85 − 7.77 (m, 2H), 7.51 (d, J = 1.9 Hz, 1H), 6.58 (d, J = 8.6 Hz, 1H), 5.29 (s, 2H), 4.26 − 4.18 (m, 1H), 4.08 (s, 4H), 2.96 (dt, J = 11.6, 2.6 Hz, 1H), 2.81 (td, J = 12.0, 3.1 Hz, 1H), 2.61 (dt, J = 11.6, 9.5 Hz, 2H), 1.85 (ddd, J = 10.8, 8.2, 3.1 Hz, 1H), 0.81 − 0.70 (m, 1H), 0.48 − 0.38 (m, 2H), 0.26 (qt, J = 3.8, 2.2 Hz, 2H). |
| 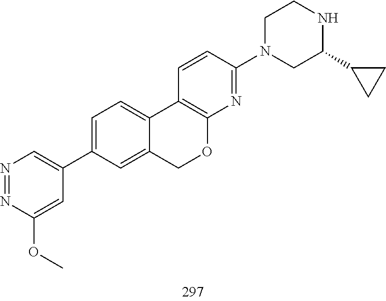 [SMILES: COc1cc(-c2ccc3c(c2)COc2nc(N4CCN[C@H](C5CC5)C4)ccc2-3)cnn1] | LCMS (ES, m/z): 416 [M + H]⁺. ¹H NMR (400 MHz, DMSO-d₆) δ 9.33 (d, J = 1.9 Hz, 1H), 8.10 (d, J = 8.6 Hz, 1H), 7.90 (dd, J = 8.2, 2.0 Hz, 1H), 7.85 − 7.77 (m, 2H), 7.51 (d, J = 1.9 Hz, 1H), 6.58 (d, J = 8.6 Hz, 1H), 5.29 (s, 2H), 4.26 − 4.18 (m, 1H), 4.08 (s, 4H), 2.96 (dt, J = 11.6, 2.6 Hz, 1H), 2.81 (td, J = 12.0, 3.1 Hz, 1H), 2.61 (dt, J = 11.6, 9.5 Hz, 2H), 1.85 (ddd, J = 10.8, 8.2, 3.1 Hz, 1H), 0.81 − 0.70 (m, 1H), 0.48 − 0.38 (m, 2H), 0.26 (qt, J = 3.8, 2.2 Hz, 2H). |
| 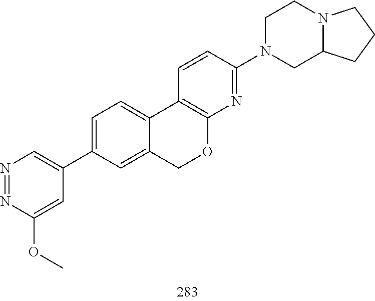 [SMILES: COc1cc(-c2ccc3c(c2)COc2nc(N4CCN5CCCC5C4)ccc2-3)cnn1] | LCMS (ES, m/z): 416 [M + H]⁺. ¹H NMR (400 MHz, DMSO-d₆) δ 9.33 (d, J = 1.9 Hz, 1H), 8.10 (d, J = 8.7 Hz, 1H), 7.90 (dd, J = 8.2, 2.1 Hz, 1H), 7.83 − 7.78 (m, 2H), 7.50 (d, J = 1.9 Hz, 1H), 6.61 (d, J = 8.7 Hz, 1H), 5.29 (s, 2H), 4.42 (dt, J = 11.9, 2.2 Hz, 1H), 4.25 (dq, J = 11.2, 2.0 Hz, 1H), 4.08 (s, 3H), 3.08 − 2.97 (m, 2H), 2.95 − 2.84 (m, 1H), 2.59 − 2.53 (m, 1H), 2.14 − 2.00 (m, 2H), 1.95 − 1.78 (m, 2H), 1.70 (dtd, J = 15.6, 11.8, 8.8 Hz, 2H), 1.42 − 1.31 (m, 1H). |
| 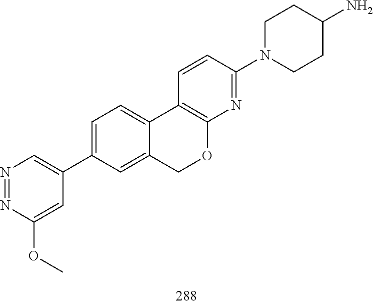 [SMILES: COc1cc(-c2ccc3c(c2)COc2nc(N4CCC(N)CC4)ccc2-3)cnn1] | LCMS (ES, m/z): 389 [M + H]⁺. ¹H NMR (400 MHz, DMSO-d₆) δ 9.33 (d, J = 1.9 Hz, 1H), 8.09 (dd, J = 8.7, 5.4 Hz, 1H), 7.90 (dd, J = 8.1, 2.0 Hz, 1H), 7.84 − 7.77 (m, 2H), 7.50 (d, J = 1.9 Hz, 1H), 6.61 (dd, J = 8.7, 5.8 Hz, 1H), 5.29 (d, J = 2.3 Hz, 2H), 4.20 (dd, J = 10.5, 6.7 Hz, 2H), 4.08 (s, 3H), 2.95 (ddd, J = 13.7, 11.4, 2.7 Hz, 2H), 2.82 (dq, J = 9.6, 4.9, 4.0 Hz, 1H), 1.75 (dd, J = 13.0, 3.7 Hz, 2H), 1.25 − 1.12 (m, 2H). |

### Example 88: Synthesis of Compound 269

### Synthesis of Compound 269

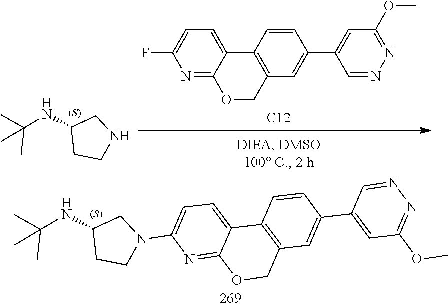

To a solution of (3S)—N-tert-butylpyrrolidin-3-amine (91.98 mg, 0.647 mmol, 1 equiv) in DMSO (2 mL) was added 5-{3-fluoro-6H-isochromeno[3,4-b]pyridin-8-yl}-3-methoxypyridazine (200 mg, 0.647 mmol, 1.00 equiv) and DIEA (250.72 mg, 1.941 mmol, 3 equiv). The reaction mixture was stirred for 2 h at 100° C. under a nitrogen atmosphere, then concentrated under reduced pressure to give a residue. The residue was purified by Prep-HPLC (Condition 10, Gradient 4) to afford (3S)—N-tert-butyl-1-[8-(6-methoxypyridazin-4-yl)-6H-isochromeno[3,4-b]pyridin-3-yl]pyrrolidin-3-amine (25.2 mg, 9%) as a solid. LCMS (ES, m/z): 432 [M+H]⁺. ¹H NMR (400 MHz, DMSO-d₆) δ 9.33 (d, J=1.9 Hz, 1H), 8.07 (d, J=8.5 Hz, 1H), 7.89 (dd, J=8.1, 2.0 Hz, 1H), 7.82 (d, J=1.9 Hz, 1H), 7.77 (d, J=8.3 Hz, 1H), 7.50 (d, J=1.9 Hz, 1H), 6.21 (d, J=8.5 Hz, 1H), 5.26 (s, 2H), 4.08 (s, 3H), 3.66 (d, J=8.6 Hz, 1H), 3.58-3.41 (m, 2H), 3.28 (s, 1H), 2.96 (t, J=8.9 Hz, 1H), 2.20-2.03 (m, 1H), 1.70 (t, J=10.3 Hz, 1H), 1.07 (s, 9H).

### Example 89: Synthesis of Compounds 285 and 309

### Synthesis of Compound 285

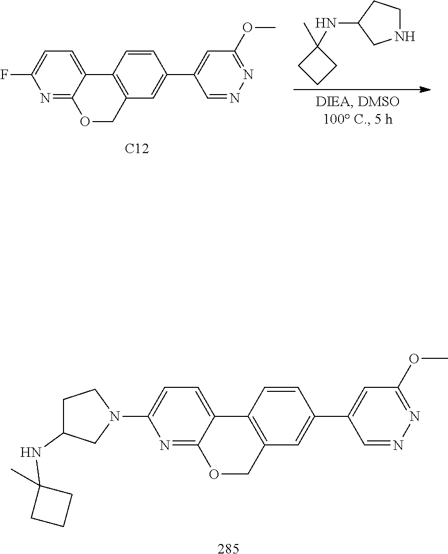

To a mixture of N-(1-methylcyclobutyl)pyrrolidin-3-amine (24.94 mg, 0.162 mmol, 1 equiv) and 5-{3-fluoro-6H-isochromeno[3,4-b]pyridin-8-yl}-3-methoxypyridazine (50 mg, 0.162 mmol, 1.00 equiv) in DMSO (2 mL, 28.158 mmol, 174.19 equiv) was added DIEA (62.68 mg, 0.486 mmol, 3 equiv). The reaction mixture was stirred for 5 h at 100° C. under a nitrogen atmosphere, then quenched with water at 0° C. The resulting mixture was extracted with ethyl acetate (3×3 mL). The organic layers were combined, then concentrated under reduced pressure to give a residue. The residue was purified by Prep-HPLC (Condition 10, Gradient 1) to afford 1-[8-(6-methoxypyridazin-4-yl)-6H-isochromeno[3,4-b]pyridin-3-yl]-N-(1-methylcyclobutyl)pyrrolidin-3-amine (6.2 mg, 9%) as a solid.

| Compound Structure | Analysis data |
|---|---|
| 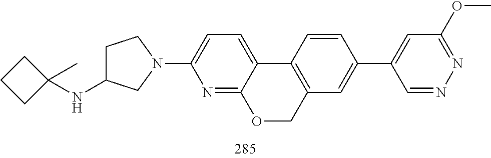 [SMILES: COc1cc(-c2ccc3c(c2)COc2nc(N4CCC(NC5(C)CCC5)C4)ccc2-3)cnn1] | LCMS (ES, m/z): 444[M + H]⁺. ¹H NMR (400 MHz, DMSO-d₆) δ 9.33 (d, J = 1.9 Hz, 1H), 8.07 (d, J = 8.6 Hz, 1H), 7.89 (dd, J = 8.2, 2.0 Hz, 1H), 7.81 (d, J = 1.9 Hz, 1H), 7.77 (d, J = 8.3 Hz, 1H), 7.50 (d, J = 1.9 Hz, 1H), 6.21 (d, J = 8.6 Hz, 1H), 5.26 (s, 2H), 4.08 (s, 3H), 3.62 (dd, J = 10.4, 6.7 Hz, 1H), 3.54 (s, 1H), 3.42 (t, J = 7.1 Hz, 1H), 3.35 (s, 1H), 3.02 (dd, J = 10.4, 6.9 Hz, 1H), 2.09 (td, J = 11.6, 6.1 Hz, 2H), 1.92 (q, J = 9.7 Hz, 2H), 1.77 (qd, J = 7.4, 2.9 Hz, 2H), 1.72 − 1.58 (m, 3H), 1.24 (s, 3H). |
| 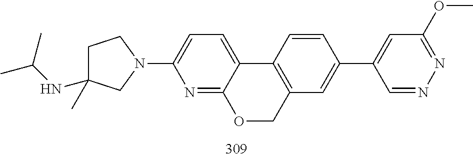 [SMILES: COc1cc(-c2ccc3c(c2)COc2nc(N4CCC(C)(NC(C)C)C4)ccc2-3)cnn1] | LCMS (ES, m/z): 432[M + H]⁺. ¹H NMR (400 MHz, DMSO-d₆) δ 9.33 (d, J = 1.9 Hz, 1H), 8.06 (d, J = 8.5 Hz, 1H), 7.89 (dd, J = 8.1, 2.0 Hz, 1H), 7.81 (d, J = 1.9 Hz, 1H), 7.76 (d, J = 8.2 Hz, 1H), 7.50 (d, J = 1.9 Hz, 1H), 6.20 (d, J = 8.5 Hz, 1H), 5.26 (s, 2H), 4.08 (s, 3H), 3.51 (s, 1H), 3.45 − 3.35 (m, 1H), 3.28 (s, 2H), 2.94 (p, J = 6.3 Hz, 1H), 1.93 (dt, J = 12.3, 7.5 Hz, 1H), 1.80 (td, J = 7.2, 3.7 Hz, 1H), 1.51 (s, 1H), 1.21 (s, 3H), 1.00 (dd, J = 10.3, 6.3 Hz, 6H). |

### Example 90: Synthesis of Compound 270

### Synthesis of Compound 270

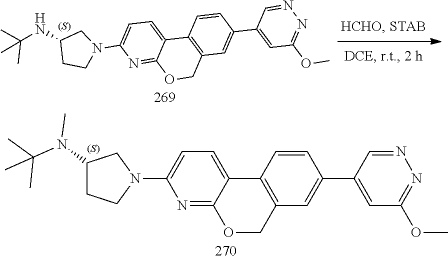

To a solution of (3S)—N-tert-butyl-1-[8-(6-methoxypyridazin-4-yl)-6H-isochromeno[3,4-b]pyridin-3-yl] pyrrolidin-3-amine (50 mg, 0.116 mmol, 1 equiv) in DCE (2 mL) was added HCHO (17.39 mg, 0.580 mmol, 5 equiv) and STAB (73.67 mg, 0.348 mmol, 3 equiv). The reaction mixture was stirred for 2 h at 25° C. under a nitrogen atmosphere, then quenched with water at 0° C. The resulting mixture was extracted with ethyl acetate (3×3 mL). The organic layers were combined, then concentrated under reduced pressure to give a residue. The residue was purified by Prep-HPLC (Condition 11, Gradient 1) to afford (3S)—N-tert-butyl-1-[8-(6-methoxypyridazin-4-yl)-6H-isochromeno[3,4-b]pyridin-3-yl]-N-methylpyrrolidin-3-amine (9.2 mg, 18%) as a solid. LCMS (ES, m/z): 446[M+H]⁺. ¹H NMR (400 MHz, DMSO-d₆) δ 9.33 (d, J 1.9 Hz, 1H), 8.08 (d, J=8.5 Hz, 1H), 7.89 (dd, J=8.2, 2.0 Hz, 1H), 7.85-7.74 (m, 2H), 7.50 (d, J=1.9 Hz, 1H), 6.25 (d, J=8.5 Hz, 1H), 5.27 (s, 2H), 4.08 (s, 3H), 3.89 (t, J=8.7 Hz, 1H), 3.57 (t, J=9.7 Hz, 1H), 3.38 (d, J=10.1 Hz, 1H), 3.31-3.24 (m, 1H), 3.19 (t, J=9.7 Hz, 1H), 2.19 (s, 3H), 2.07-1.95 (m, 1H), 1.87 (s, 1H), 1.09 (s, 9H).

### Example 91: Synthesis of Compound 272

### Synthesis of Intermediate C76

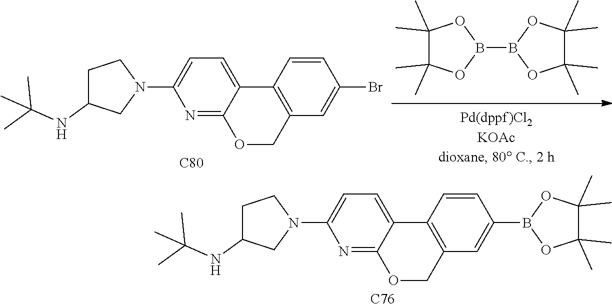

A mixture of 1-{8-bromo-6H-isochromeno[3,4-b]pyridin-3-yl}-N-tert-butylpyrrolidin-3-amine (100 mg, 0.249 mmol, 1 equiv), bis(pinacolato)diboron (63.12 mg, 0.249 mmol, 1 equiv), Pd(dppf)Cl₂ (20.25 mg, 0.025 mmol, 0.1 equiv), KOAc (73.18 mg, 0.747 mmol, 3 equiv) and 1,4-dioxane (2 mL) was stirred for 2 h at 80° C. The resulting mixture was filtered and the filter cake was washed with CH₂Cl₂ (3×5 mL). The filtrate was concentrated under reduced pressure to afford N-(tert-butyl)-1-(8-(4,4,5,5-tetramethyl-1,3,2-dioxaborolan-2-yl)-6H-isochromeno[3,4-b]pyridin-3-yl)pyrrolidin-3-amine (110 mg) as an oil.

### Synthesis of Compound 272

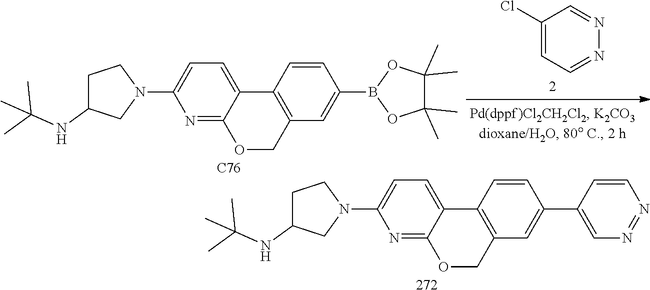

A solution of N-tert-butyl-1-[8-(4,4,5,5-tetramethyl-1,3,2-dioxaborolan-2-yl)-6H-isochromeno[3,4-b]pyridin-3-yl]pyrrolidin-3-amine (110 mg, 0.196 mmol, 1 equiv, 80%) in a mixture of 1,4-dioxane (3.2 mL) and water (0.8 mL) was treated with 4-chloropyridazine (33.64 mg, 0.294 mmol, 1.5 equiv), Pd(dppf)Cl₂CH₂Cl₂ (15.95 mg, 0.020 mmol, 0.1 equiv), and K₂CO₃ (81.19 mg, 0.588 mmol, 3 equiv). The reaction mixture was stirred for 2 h at 80° C. under nitrogen atmosphere. The resulting mixture was filtered and the filter cake was washed with ethyl acetate (3×5 mL). The filtrate was concentrated under reduced pressure to give a residue. The residue was purified by chiral-Prep-HPLC (Column, XBridge Shield RP18 OBD Column, 30*150 mm, 5 μm; mobile phase, Water (10 mmol/L NH₄HCO₃) and ACN (15% ACN up to 40% in 10 min); detector, UV 254 nm) to afford N-tert-butyl-1-[8-(pyridazin-4-yl)-6H-isochromeno[3,4-b]pyridin-3-yl]pyrrolidin-3-amine (4.3 mg, 5%) as a solid. LCMS (ES, m/z): 402[M+H]+. 1H NMR (400 MHz, DMSO-d₆) δ 9.66 (dd, J=2.7, 1.3 Hz, 1H), 9.25 (dd, J=5.5, 1.2 Hz, 1H), 8.09 (d, J=8.4 Hz, 1H), 8.01 (dd, J=5.5, 2.5 Hz, 1H), 7.91 (dd, J=8.1, 2.0 Hz, 1H), 7.86-7.77 (m, 2H), 6.24 (d, J=8.4 Hz, 1H), 5.28 (s, 2H), 3.70 (s, 1H), 3.53 (s, 1H), 3.30 (s, 2H), 2.98 (s, 1H), 2.15 (s, 1H), 1.73 (s, 1H), 1.10 (s, 9H).

### Example 92: Synthesis of Compound 266

### Synthesis of Intermediate C80

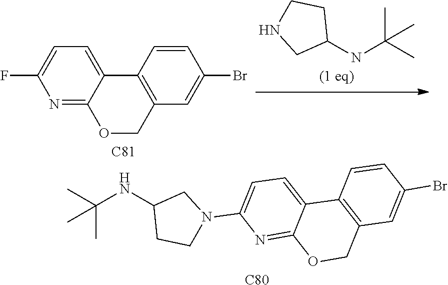

A mixture of 8-bromo-3-fluoro-6H-isochromeno[3,4-b]pyridine (700 mg, 2.499 mmol, 1 equiv), N-tert-butylpyrrolidin-3-amine (356 mg, 2.499 mmol, 1 equiv), and DIEA (969 mg, 7.497 mmol, 3 equiv) in DMSO (7 mL) was stirred for 3 h at 90° C. The reaction mixture was cooled to room temperature, then poured into ice water (20 mL). The resulting mixture was washed with petroleum ether (1×20 mL). A solid precipitated that was collected by filtration and washed with water (1×5 mL) to afford 1-{8-bromo-6H-isochromeno[3,4-b]pyridin-3-yl}-N-tert-butylpyrrolidin-3-amine (641 mg, 64%) as a solid. LCMS (ES, m/z): 402 [M+H]⁺.

### Synthesis of Intermediate C76

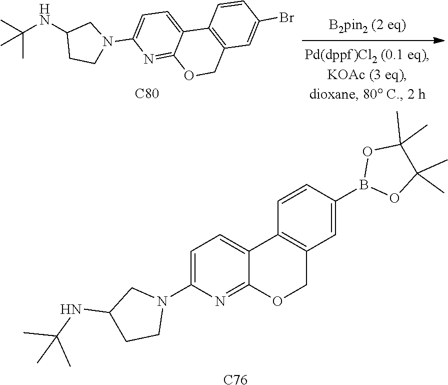 [SMILES: CC(C)(C)NC1CCN(c2ccc3c(n2)OCc2cc(Br)ccc2-3)C1 ; "C80" ; CC(C)(C)NC1CCN(c2ccc3c(n2)OCc2cc(B4OC(C)(C)C(C)(C)O4)ccc2-3)C1 ; "C76"]

A mixture of 1-{8-bromo-6H-isochromeno[3,4-b]pyridin-3-yl}-N-tert-butylpyrrolidin-3-amine (641 mg, 1.593 mmol, 1 equiv), Pd(dppf)Cl₂·CH₂Cl₂ (129.78 mg, 0.159 mmol, 0.1 equiv), 4,4,5,5-tetramethyl-2-(tetramethyl-1,3,2-dioxaborolan-2-yl)-1,3,2-dioxaborolane (809.15 mg, 3.186 mmol, 2 equiv), and KOAc (469.08 mg, 4.779 mmol, 3 equiv) in 1,4-dioxane (6.4 mL) was stirred for 2 h at 80° C. under nitrogen atmosphere. The reaction mixture was cooled to room temperature, then poured into water. The resulting mixture was extracted with ethyl acetate (1×50 mL). The organic layer was dried over anhydrous Na₂SO₄, then filtered. The filtrate was concentrated under reduced pressure to afford N-tert-butyl-1-[8-(4,4,5,5-tetramethyl-1,3,2-dioxaborolan-2-yl)-6H-isochromeno[3,4-b]pyridin-3-yl]pyrrolidin-3-amine (720 mg, 100%) as an oil. LCMS (ES, m/z): 450 [M+H]⁺.

### Synthesis of Compound 266

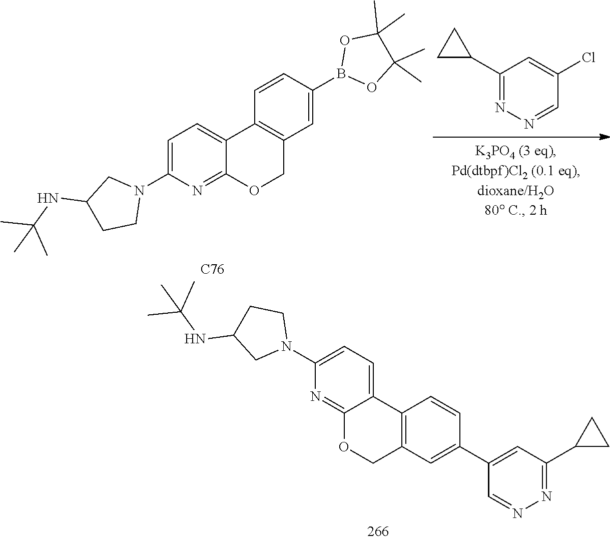

A mixture of 5-chloro-3-cyclopropylpyridazine (16 mg, 0.103 mmol, 1 equiv), N-tert-butyl-l-[8-(4,4,5,5-tetramethyl-1,3,2-dioxaborolan-2-yl)-6H-isochromeno[3,4-b]pyridin-3-yl]pyrrolidin-3-amine (47 mg, 0.103 mmol, 1 equiv), K₃PO₄ (66 mg, 0.309 mmol, 3 equiv), and Pd(DtBPF)Cl₂ (7 mg, 0.010 mmol, 0.1 equiv) in a mixture of 1,4-dioxane (0.25 mL) and water (0.32 mL) was stirred for 2 h at 80° C. under nitrogen atmosphere. The reaction mixture was cooled to room temperature, then extracted with ethyl acetate (1×20 mL). The organic layer was dried over anhydrous Na₂SO₄, then filtered. The filtrate was concentrated under reduced pressure to give a residue. The residue was purified by silica gel column chromatography, eluted with CH₂Cl₂/MeOH (10:1), followed by Prep-HPLC (Condition 11, Gradient 4) to afford N-tert-butyl-1-[8-(6-cyclopropylpyridazin-4-yl)-6H-isochromeno[3,4-b]pyridin-3-yl]pyrrolidin-3-amine (3.5 mg, 8%) as a solid. LCMS (ES, m/z): 442 [M+H]⁺. ¹H NMR (400 MHz, DMSO-d₆) δ 9.41 (d, J=2.2 Hz, 1H), 8.07 (d, J=8.6 Hz, 1H), 7.89 (dd, J=8.2, 2.0 Hz, 1H), 7.84-7.82 (d, 1H), 7.75 (m, 2H), 6.21 (d, J=8.5 Hz, 1H), 5.27 (s, 2H), 3.66 (d, J=9.1 Hz, 1H), 3.53 (s, 2H), 3.28 (s, 1H), 2.97 (t, J=8.9 Hz, 1H), 2.33-2.24 (m, 1H), 2.13 (s, 1H), 1.70 (t, J=10.0 Hz, 1H), 1.17 (m, 3H), 1.09 (m, 1H), 1.08 (s, 9H).

### Example 93: Synthesis of Compound 267

### Synthesis of Compound 267

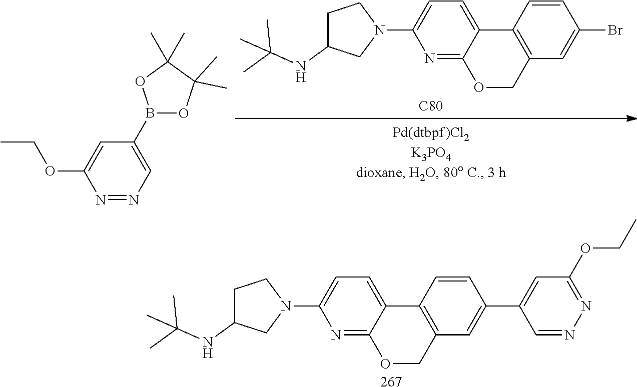

A mixture of 3-ethoxy-5-(4,4,5,5-tetramethyl-1,3,2-dioxaborolan-2-yl)pyridazine (100 mg, 0.400 mmol, 1 equiv), 1-{8-bromo-6H-isochromeno[3,4-b]pyridin-3-yl}-N-tert-butylpyrrolidin-3-amine (160.86 mg, 0.400 mmol, 1 equiv), Pd(dtbpf)Cl₂ (26.06 mg, 0.040 mmol, 0.1 equiv), and K₃PO₄ (254.61 mg, 1.200 mmol, 3 equiv) in a mixture of dioxane (4 mL) and water (1 mL) was stirred for 3 h at 80° C. The aqueous layer was extracted with ethyl acetate (3×5 mL). The organic layers were combined, dried over anhydrous Na₂SO₄, and filtered. The filtrate was concentrated under reduced pressure to give a residue. The residue was purified by Chiral-Prep-HPLC (Condition 13, Gradient 1) to afford N-tert-butyl-1-[8-(6-ethoxypyridazin-4-yl)-6H-isochromeno [3,4-b]pyridin-3-yl]pyrrolidin-3-amine (6.3 mg, 4%) as a solid. LCMS (ES, m/z): 446[M+H]+. 1H NMR (400 MHz, DMSO-d₆) δ 9.31 (d, J=1.9 Hz, 1H), 8.06 (d, J=8.5 Hz, 1H), 7.89 (dd, J=8.2, 2.0 Hz, 1H), 7.82 (s, 1H), 7.76 (d, J=8.2 Hz, 1H), 7.47 (d, J=1.8 Hz, 1H), 6.21 (d, J=8.4 Hz, 1H), 5.26 (s, 2H), 4.53 (q, J=7.0 Hz, 2H), δ 3.67 (t, J=8.7 Hz, 1H), 3.53-3.43 (m, 3H), 2.99-2.94 (m, 1H), 2.15-2.08 (m, 1H), 1.68 (d, J=8.6 Hz, 1H), 1.41 (t, J=7.0 Hz, 3H), 1.07 (s, 9H).

### Example 94: Synthesis of Compound 268

### Synthesis of Intermediate C82

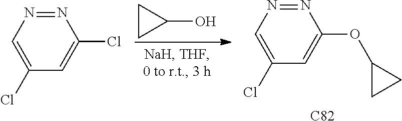

To a mixture of cyclopropanol (1559.51 mg, 26.851 mmol, 1 equiv) and THF (40.01 mL, 493.790 mmol, 18.39 equiv) was added NaH (48.33 mg, 2.014 mmol, 1.5 equiv) at 0° C. The resulting mixture was stirred for 30 min at 0° C. To the resulting mixture was added 3,5-dichloropyridazine (4 g, 26.851 mmol, 1 equiv) in THE (10 mL) dropwise at 0° C. The resulting mixture was stirred for 3 h at 0° C., then quenched with a mixture of water and ice (50 mL). The resulting mixture was extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with PE/EA (4:1) to afford 5-chloro-3-cyclopropoxypyridazine (1.2 g, 26%) as an oil. ¹H NMR (400 MHz, DMSO-d₆) δ 9.08 (d, J=2.0 Hz, 1H), 7.59 (d, J=2.0 Hz, 1H), 4.42 (tt, J=6.3, 3.0 Hz, 1H), 0.87-0.81 (m, 2H), 0.79-0.72 (m, 2H).

### Synthesis of Intermediate C83

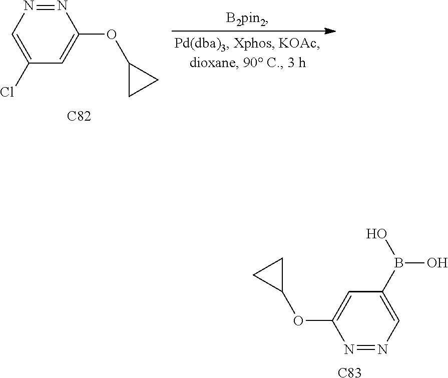 [SMILES: Clc1cnnc(OC2CC2)c1 ; "C82" ; OB(O)c1cnnc(OC2CC2)c1 ; "C83"]

A mixture of 5-chloro-3-cyclopropoxypyridazine (200 mg, 1.172 mmol, 1 equiv), 4,4,5,5-tetramethyl-2-(tetramethyl-1,3,2-dioxaborolan-2-yl)-1,3,2-dioxaborolane (595.40 mg, 2.344 mmol, 2 equiv), KOAc (345.17 mg, 3.516 mmol, 3 equiv), Pd₂(dba)₃ (107.35 mg, 0.117 mmol, 0.1 equiv), Xphos (111.78 mg, 0.234 mmol, 0.2 equiv) and dioxane (6.67 mL, 78.676 mmol, 67.13 equiv) was stirred for 3 h at 80° C. under nitrogen atmosphere, then cooled to room temperature. The resulting mixture was filtered and the filter cake was washed with ethyl acetate (3×10 mL). The filtrate was concentrated under reduced pressure to afford product. LCMS (ES, m/z): 181 [M+H]+.

### Synthesis of Compound 268

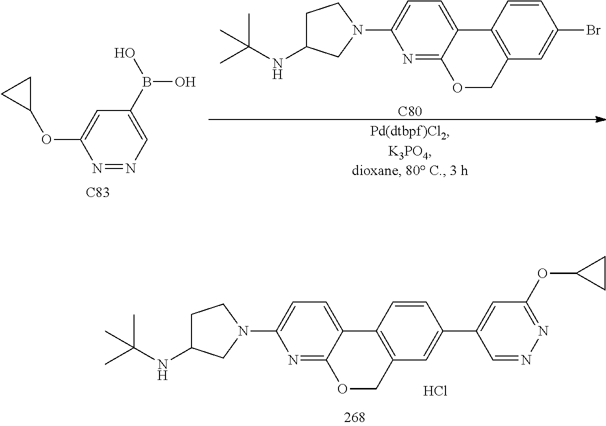

A mixture of 3-cyclopropoxy-5-(4,4,5,5-tetramethyl-1,3,2-dioxaborolan-2-yl)pyridazine (200 mg, 0.252 mmol, 1 equiv, 33%), dioxane (5 mL), and K₃PO₄ (160.34 mg, 0.756 mmol, 3 equiv) was stirred for 3 h at 80° C. under nitrogen atmosphere. The resulting mixture was concentrated under reduced pressure to give a residue. The residue was purified by Prep-TLC (CH₂Cl₂/MeOH 10:1), followed by Prep-HPLC (Condition 14, Gradient 1) to afford N-tert-butyl-1-[8-(6-cyclopropoxypyridazin-4-yl)-6H-isochromeno[3,4-b]pyridin-3-yl]pyrrolidin-3-amine (11.9 mg, 10%) as a solid. LCMS (ES, m/z): 458[M+H]+. ¹H NMR (400 MHz, DMSO-d₆) δ 9.37 (d, J=1.9 Hz, 1H), 8.74 (s, 2H), 8.16 (d, J=8.5 Hz, 1H), 7.91 (dd, J=8.1, 2.1 Hz, 1H), 7.86-7.79 (m, 2H), 7.52 (d, J=1.9 Hz, 1H), 6.34 (d, J=8.5 Hz, 1H), 5.29 (s, 2H), 4.47 (dq, J=6.2, 3.0 Hz, 1H), 4.10 (s, 1H), 3.87 (dd, J=11.3, 6.9 Hz, 1H), 3.65-3.60 (m, 1H), 3.59-3.51 (m, 2H), 2.41 (s, 1H), 2.19 (d, J=11.0 Hz, 1H), 1.36 (s, 9H), 0.87 (q, J=6.6, 5.8 Hz, 2H), 0.77 (s, 2H).

### Example 95: Synthesis of Compound 281

### Synthesis of Compound 281

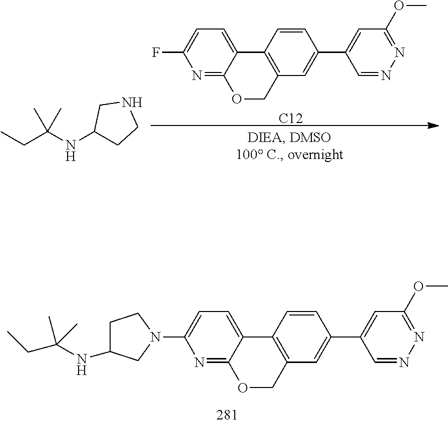

A mixture of N-(2-methylbutan-2-yl)pyrrolidin-3-amine (50 mg, 0.320 mmol, 1 equiv), 5-{3-fluoro-6H-isochromeno[3,4-b]pyridin-8-yl}-3-methoxypyridazine (98.96 mg, 0.320 mmol, 1 equiv), DIEA (124.06 mg, 0.960 mmol, 3 equiv), and DMSO (2 mL) was stirred for 3 h at 100° C., then cooled to room temperature. The resulting mixture was quenched with a mixture of water and ice (10 mL) at room temperature. A solid precipitated that was collected by filtration and washed with water (3×3 mL). The solid was purified by Prep-HPLC (Condition 10, Gradient 2) to afford 1-[8-(6-methoxypyridazin-4-yl)-6H-isochromeno[3,4-b]pyridin-3-yl]-N-(2-methylbutan-2-yl)pyrrolidin-3-amine (10.4 mg, 7%) as a solid. LCMS (ES, m/z): 446[M+H]⁺. ¹H NMR (400 MHz, DMSO-d₆) δ 9.33 (d, J=1.9 Hz, 1H), 8.08 (d, J=8.5 Hz, 1H), 7.90 (dd, J=8.3, 2.0 Hz, 1H), 7.82 (d, J=1.8 Hz, 1H), 7.77 (d, J=8.3 Hz, 1H), 7.50 (d, J=1.9 Hz, 1H), 6.22 (d, J=8.4 Hz, 1H), 5.27 (s, 2H), 4.08 (s, 3H), 3.67 (s, 1H), 3.53 (s, 1H), 3.46-3.38 (m, 1H), 3.30 (s, 1H), 2.97 (s, 1H), 2.13 (s, 1H), 1.72 (s, 1H), 1.38 (s, 2H), 1.01 (s, 6H), 0.83 (t, J=7.5 Hz, 3H).

### Example 96: Synthesis of Compound 315

### Synthesis of Intermediate C84

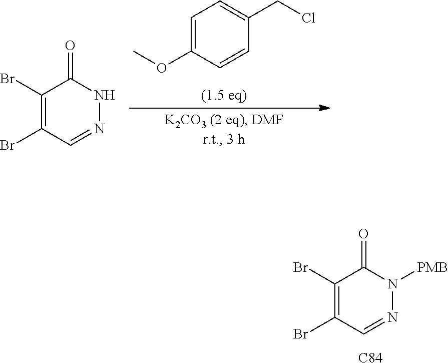

A mixture of 4,5-dibromo-2H-pyridazin-3-one (9 g, 35.450 mmol, 1 equiv), 4-methoxybenzyl chloride (8.33 g, 53.175 mmol, 1.5 equiv), and K₂CO₃ (9.80 g, 70.900 mmol, 2 equiv) in DMF (90 mL) was stirred for 3 h at room temperature. The resulting mixture was poured into water, then extracted with ethyl acetate (1×400 mL). The organic layers were combined, washed with brine (3×400 mL), dried over anhydrous Na₂SO₄, and filtered. After filtration, the filtrate was concentrated under reduced pressure to give a residue. The residue was purified by trituration with isopropanol (50 mL) to afford 4,5-dibromo-2-[(4-methoxyphenyl)methyl]pyridazin-3-one (9 g, 68%) as a solid.

### Synthesis of Intermediate C85

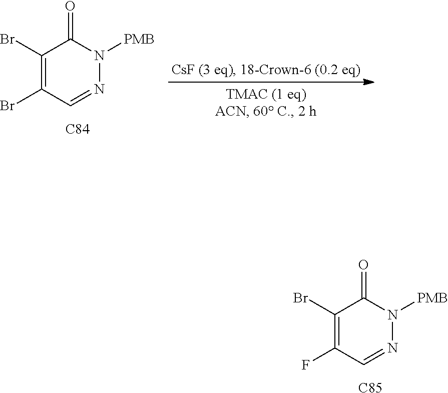

A solution of 4,5-dibromo-2-[(4-methoxyphenyl)methyl]pyridazin-3-one (9 g, 24.062 mmol, 1 equiv) in acetonitrile (90 mL) was treated with 18-Crown-6 (1.27 g, 4.812 mmol, 0.2 equiv) and tetramethylazanium chloride (2.64 g, 24.062 mmol, 1 equiv). The reaction mixture was stirred for 10 min at 60° C. under nitrogen atmosphere. To the resulting mixture was added CsF (10.97 g, 72.186 mmol, 3 equiv) in portions at 60° C. The resulting mixture was stirred for 2 h at 60° C. under nitrogen atmosphere, then filtered and the filter cake washed with acetonitrile (1×20 mL). The filtrate was concentrated under reduced pressure to give a residue. The residue was purified by silica gel column chromatography, eluted with PE/EA (1:1) to afford 4-bromo-5-fluoro-2-[(4-methoxyphenyl)methyl]pyridazin-3-one (6.5 g, 86%) as a solid.

### Synthesis of Intermediate C86

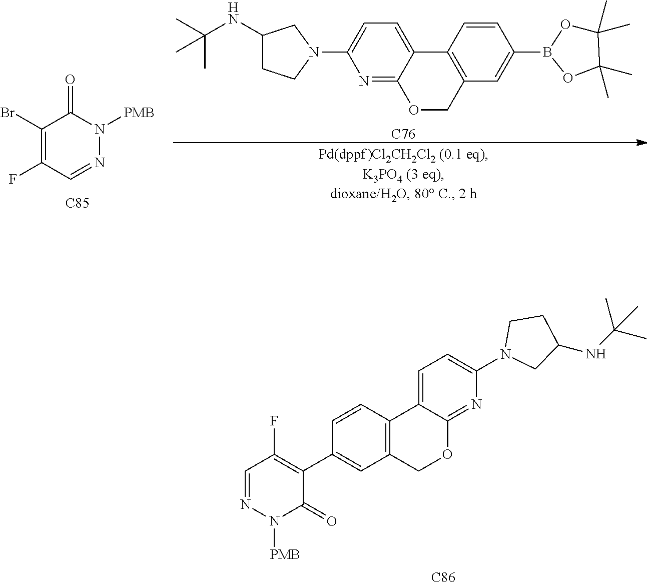

A mixture of 4-bromo-5-fluoro-2-[(4-methoxyphenyl)methyl]pyridazin-3-one (1 g, 3.194 mmol, 1 equiv), N-tert-butyl-1-[8-(4,4,5,5-tetramethyl-1,3,2-dioxaborolan-2-yl)-6H-isochromeno[3,4-b]pyridin-3-yl]pyrrolidin-3-amine (1.44 g, 3.194 mmol, 1 equiv), Pd(dppf)Cl₂CH₂Cl₂ (0.26 g, 0.319 mmol, 0.1 equiv), and K₃PO₄ (2.03 g, 9.582 mmol, 3 equiv) in a mixture of 1,4-dioxane (8 mL) and water (2 mL) was stirred for 2 h at 80° C. under nitrogen atmosphere, then cooled to room temperature. The resulting mixture was extracted with ethyl acetate (1×100 mL). The organic layer was dried over anhydrous Na₂SO₄ and filtered. After filtration, the filtrate was concentrated under reduced pressure to give a residue. The residue was purified by silica gel column chromatography, eluted with PE/EA (1:1) to afford 4-{3-[3-(tert-butylamino)pyrrolidin-1-yl]-6H-isochromeno[3,4-b]pyridin-8-yl}-5-fluoro-2-[(4-methoxyphenyl)methyl]pyridazin-3-one (436 mg, 25%) as a solid.

### Synthesis of Compound 315

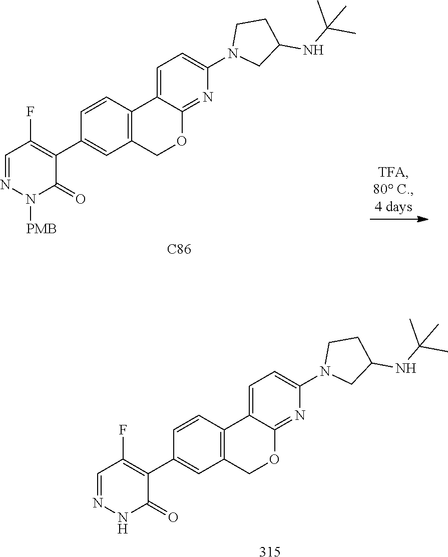

A solution of 4-{3-[3-(tert-butylamino)pyrrolidin-1-yl]-6H-isochromeno[3,4-b]pyridin-8-yl}-5-fluoro-2-[(4-methoxyphenyl)methyl]pyridazin-3-one (65.4 mg, 0.118 mmol, 1 equiv) in TFA (6.5 mL) was stirred for 4 days at 80° C., then cooled to room temperature. The resulting mixture was concentrated under reduced pressure to give a residue. The residue was purified by Prep-HPLC (Condition 11, Gradient 1) to afford 4-{3-[3-(tert-butylamino)pyrrolidin-1-yl]-6H-isochromeno[3,4-b]pyridin-8-yl}-5-fluoro-2H-pyridazin-3-one (12.7 mg, 24%) as a solid. LCMS (ES, m/z): 436 [M+H]⁺. ¹H NMR (400 MHz, Chloroform-d) δ 7.90 (s, 1H), 7.80 (d, J=8.5 Hz, 1H), 7.63 (dt, J=8.2, 1.7 Hz, 1H), 7.55 (d, 1H), 7.46 (d, 1H), 6.10 (d, J=8.5 Hz, 1H), 5.30 (s, 2H), 3.84 (d, J=9.2 Hz, 1H), 3.69 (s, 1H), 3.63-3.51 (m, 1H), 3.44 (td, J=9.5, 7.1 Hz, 1H), 3.17 (t, J=8.8 Hz, 1H), 2.27 (s, 1H), 1.88 (d, J=12.3 Hz, 1H), 1.19 (s, 9H).

### Example 97: Synthesis of Compounds 275 and 280

### Synthesis of Intermediate C87

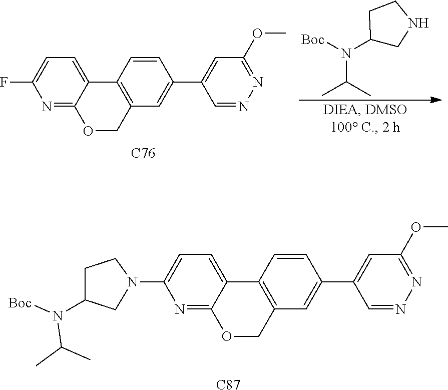

A mixture of tert-butyl N-isopropyl-N-(pyrrolidin-3-yl)carbamate (80 mg, 0.350 mmol, 1 equiv), 5-{3-fluoro-6H-isochromeno[3,4-b]pyridin-8-yl}-3-methoxypyridazine (108.37 mg, 0.350 mmol, 1.00 equiv), DIEA (135.85 mg, 1.050 mmol, 3 equiv), and DMSO (2 mL) was stirred for 2 h at 100° C., then quenched with water (3 mL) at room temperature. The aqueous layer was extracted with ethyl acetate (3×3 mL). The organic layers were combined, dried over anhydrous Na₂SO₄, and filtered. After filtration, the filtrate was concentrated under reduced pressure to give a residue. The residue was purified by Prep-TLC (PE/EA 1:1) to afford tert-butyl N-isopropyl-N-{1-[8-(6-methoxypyridazin-4-yl)-6H-isochromeno[3,4-b]pyridin-3-yl]pyrrolidin-3-yl}carbamate (160 mg, 88%) as a solid.

### Synthesis of Compound 275

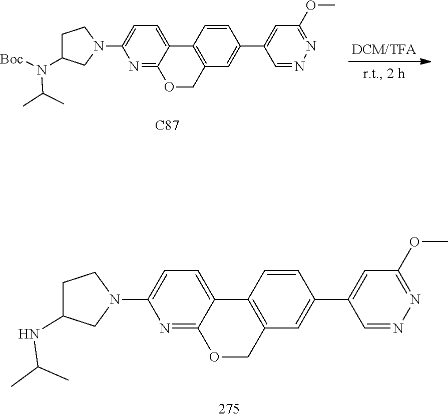

Tert-butyl N-isopropyl-N-{1-[8-(6-methoxypyridazin-4-yl)-6H-isochromeno[3,4-b]pyridin-3-yl]pyrrolidin-3-yl}carbamate (160 mg, 0.309 mmol, 1 equiv), TFA (0.5 mL), and DCM (2 mL) were stirred at room temperature. The resulting mixture was concentrated under reduced pressure to give a residue. The residue was purified by Prep-HPLC (Condition 10, Gradient 5) to afford N-isopropyl-1-[8-(6-methoxypyridazin-4-yl)-6H-isochromeno[3,4-b]pyridin-3-yl]pyrrolidin-3-amine (9.2 mg, 70%) as a solid.

| Compound Structure | Analysis data |
|---|---|
| 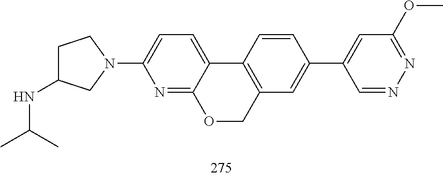 [SMILES: COc1cc(-c2ccc3c(c2)COc2nc(N4CCC(NC(C)C)C4)ccc2-3)cnn1] | LCMS (ES, m/z):418[M + H]⁺. ¹H NMR (400 MHz, DMSO-d₆) δ 9.33 (d, J = 2.0 Hz, 1H), 8.07 (d, J = 8.6 Hz, 1H), 7.89 (dd, J = 8.2, 2.0 Hz, 1H), 7.82 (d, J = 1.9 Hz, 1H), 7.77 (d, J = 8.3 Hz, 1H), 7.50 (d, J = 1.9 Hz, 1H), 6.22 (d, J = 8.5 Hz, 1H), 5.26 (s, 2H), 4.08 (s, 3H), δ 3.64 − 3.60 (m, 1H), 3.52 (s, 1H), 3.41 − 3.51 (m, 1H), 3.34 − 3.41 (m, 1H), 3.11 − 3.07 (m, 1H), 2.85 − 2.79 (m, 1H), 2.11 − 2.07 (m, 1H), 1.74 (s, 1H), 1.00 (dd, J = 6.2, 4.8 Hz, 6H). |
| 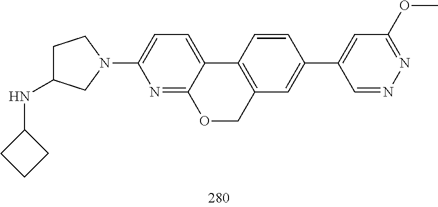 [SMILES: COc1cc(-c2ccc3c(c2)COc2nc(N4CCC(NC5CCC5)C4)ccc2-3)cnn1] | LCMS (ES, m/z):430[M + H] *. ¹H NMR (400 MHz, DMSO-d₆) δ 9.33 (d, J = 1.9 Hz, 1H), 8.08 (d, J = 8.5 Hz, 1H), 7.89 (dd, J = 8.1, 2.0 Hz, 1H), 7.82 (d, J = 2.0 Hz, 1H), 7.77 (d, J = 8.2 Hz, 1H), 7.50 (d, J = 1.9 Hz, 1H), 6.22 (d, J = 8.5 Hz, 1H), 5.27 (s, 2H), 4.08 (s, 3H), δ 3.51 (tt, J = 14.6, 6.7 Hz, 2H), 3.41 − 3.35 (m, 2H), 3.26 (d, J = 1.6 Hz, 1H), 3.20 − 3.10 (m, 1H), 2.20 − 2.08 (m, 2H), 2.05 (q, J = 6.2 Hz, 1H), 1.82 − 1.64 (m, 3H), 1.63 − 1.51 (m, 3H). |

### Example 98: Synthesis of Compound 289

### Synthesis of Intermediate C88

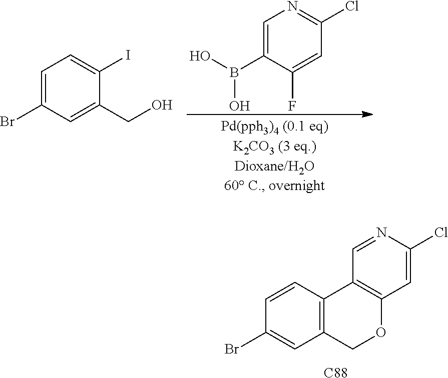 [SMILES: OCc1cc(Br)ccc1I ; OB(O)c1cnc(Cl)cc1F ; Clc1cc2c(cn1)-c1ccc(Br)cc1CO2 ; "C88"]

A mixture of (5-bromo-2-iodophenyl)methanol (1 g, 3.196 mmol, 1.00 equiv), 6-chloro-4-fluoropyridin-3-ylboronic acid (0.28 g, 1.598 mmol, 0.5 equiv), Pd(PPh₃)₄ (0.37 g, 0.320 mmol, 0.1 equiv), and K₂CO₃ (1.32 g, 9.588 mmol, 3 equiv) in a mixture of 1,4-dioxane and water (5:1) was stirred for overnight at 60° C. under nitrogen atmosphere. The reaction mixture was concentrated under reduced pressure to give a residue. The residue was purified by reverse flash chromatography (Condition 15, Gradient 1) to afford 8-bromo-3-chloro-6H-isochromeno[4,3-c]pyridine as a solid (100 mg, 11%). LCMS (ES, m/z): 297 [M+H]+.

### Synthesis of Intermediate C89

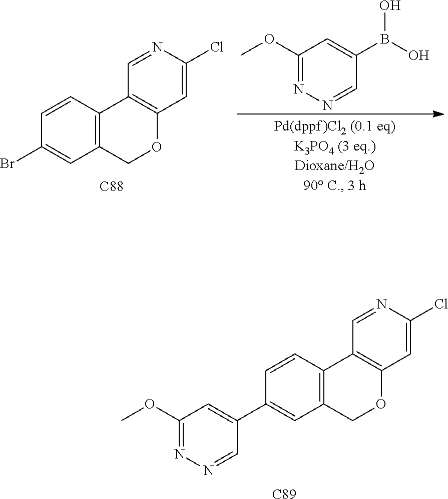

A mixture of 8-bromo-3-chloro-6H-isochromeno[4,3-c]pyridine (100 mg, 0.337 mmol, 1 equiv), 6-methoxypyridazin-4-ylboronic acid (77.86 mg, 0.506 mmol, 1.5 equiv), and K₃PO₄ (214.73 mg, 1.011 mmol, 3 equiv) in a mixture of 1,4-dioxane and water (5:1, 4 mL) was stirred for 3 h at 90° C. under nitrogen atmosphere. The resulting mixture was filtered and the filter cake washed with 1,4-dioxane (2×20 mL). The filtrate was concentrated under reduced pressure to give a residue. The residue was purified by Prep-TLC (PE/EA 1:1) to afford 5-{3-chloro-6H-isochromeno[4,3-c]pyridin-8-yl}-3-methoxypyridazine (50 mg, 46%) as a solid. LCMS (ES, m/z): 326 [M+H]+.

### Synthesis of Compound 289

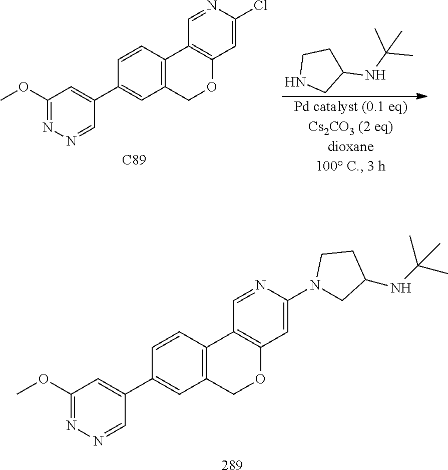

A mixture of 5-{3-chloro-6H-isochromeno[4,3-c]pyridin-8-yl}-3-methoxypyridazine (30 mg, 0.092 mmol, 1 equiv), N-tert-butylpyrrolidin-3-amine (15.72 mg, 0.110 mmol, 1.2 equiv), and Pd-PEPPSI-IPentCl 2-methylpyridine (o-picoline) (7.75 mg, 0.009 mmol, 0.1 equiv) in 1,4-dioxane was stirred for 4 h at 100° C. under nitrogen atmosphere, then filtered. The filtrate was concentrated under reduced pressure to give a residue. The residue was purified by Prep-HPLC (Condition 11, Gradient 5) to afford N-(tert-butyl)-1-(8-(6-methoxypyridazin-4-yl)-6H-isochromeno [4,3-c]pyridin-3-yl)pyrrolidin-3-amine (5.9 mg, 9%) as a solid. LCMS (ES, m/z): 432 [M+H]+. 1H NMR (400 MHz, DMSO-d₆) δ 9.33 (d, J=1.9 Hz, 1H), 8.67 (s, 1H), 7.92 (s, 2H), 7.82 (s, 1H), 7.51 (d, J=1.9 Hz, 1H), 5.96 (s, 1H), 5.20 (s, 2H), 4.08 (s, 3H), 3.70 (s, 1H), 3.52 (s, 2H), 3.2 (s, 1H), 2.99 (s, 1H), 2.14 (s, 1H), 1.71 (s, 1H), 1.08 (s, 9H).

### Example 99: Synthesis of Compound 268

### Synthesis of Intermediate C90

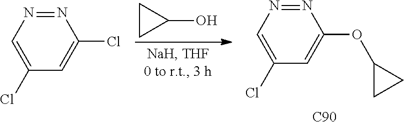

To a mixture of cyclopropanol (1559.51 mg, 26.851 mmol, 1 equiv) and THF (40.01 mL, 493.790 mmol, 18.39 equiv) was added NaH (48.33 mg, 2.014 mmol, 1.5 equiv) at 0° C. The resulting mixture was stirred for 0.5 h at 0° C. To the reaction mixture was added a solution of 3,5-dichloropyridazine (4 g, 26.851 mmol, 1 equiv) in THF (10 mL) dropwise at 0° C. The resulting mixture was stirred for 3 h at 0° C., then quenched with a mixture of water and ice (50 mL) at room temperature. The resulting mixture was extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with PE/EA (4:1) to afford 5-chloro-3-cyclopropoxypyridazine (1.2 g, 26%) as an oil. ¹H NMR (400 MHz, DMSO-d₆) δ 9.08 (d, J=2.0 Hz, 1H), 7.59 (d, J=2.0 Hz, 1H), 4.42 (tt, J=6.3, 3.0 Hz, 1H), 0.87-0.81 (m, 2H), 0.79-0.72 (m, 2H).

### Synthesis of Intermediate C91

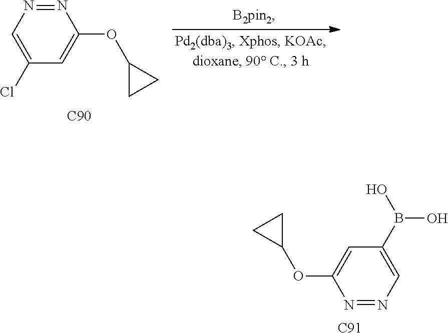 [SMILES: Clc1cnnc(OC2CC2)c1 ; "C90" ; OB(O)c1cnnc(OC2CC2)c1 ; "C91"]

A mixture of 5-chloro-3-cyclopropoxypyridazine (200 mg, 1.172 mmol, 1 equiv), 4,4,5,5-tetramethyl-2-(tetramethyl-1,3,2-dioxaborolan-2-yl)-1,3,2-dioxaborolane (595.40 mg, 2.344 mmol, 2 equiv), KOAc (345.17 mg, 3.516 mmol, 3 equiv), Pd₂(dba)₃ (107.35 mg, 0.117 mmol, 0.1 equiv), Xphos (111.78 mg, 0.234 mmol, 0.2 equiv) and dioxane (6.67 mL, 78.676 mmol, 67.13 equiv) was stirred for 3 h at 80° C. under nitrogen atmosphere. The resulting mixture was cooled to room temperature, then filtered, and the filter cake washed with ethyl acetate (3×10 mL). The filtrate was concentrated under reduced pressure to afford product. LCMS (ES, m/z): 181[M+H]⁺.

### Synthesis of Compound 268

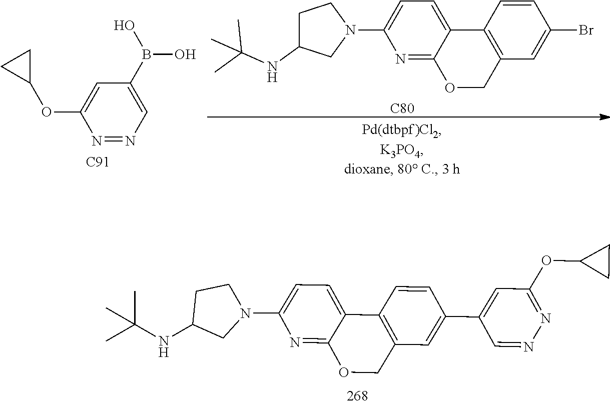

A mixture of 3-cyclopropoxy-5-(4,4,5,5-tetramethyl-1,3,2-dioxaborolan-2-yl)pyridazine (200 mg, 0.252 mmol, 1 equiv, 33%), dioxane (5 mL), K₃PO₄ (160.34 mg, 0.756 mmol, 3 equiv) was stirred for 3 h at 80° C. under nitrogen atmosphere. The resulting mixture was concentrated under reduced pressure to give a residue. The residue was purified by Prep-TLC (CH₂Cl₂/MeOH 10:1), followed by Prep-HPLC with the following conditions (Condition 14, Gradient 1) to afford N-tert-butyl-1-[8-(6-cyclopropoxypyridazin-4-yl)-6H-isochromeno[3,4-b]pyridin-3-yl]pyrrolidin-3-amine (11.9 mg, 10%) as a solid. LCMS (ES, m/z): 458[M+H]⁺. ¹H NMR (400 MHz, DMSO-d₆) δ 9.37 (d, J=1.9 Hz, 1H), 8.74 (s, 2H), 8.16 (d, J=8.5 Hz, 1H), 7.91 (dd, J=8.1, 2.1 Hz, 1H), 7.86-7.79 (m, 2H), 7.52 (d, J=1.9 Hz, 1H), 6.34 (d, J=8.5 Hz, 1H), 5.29 (s, 2H), 4.47 (dq, J=6.2, 3.0 Hz, 1H), 4.10 (s, 1H), 3.87 (dd, J=11.3, 6.9 Hz, 1H), 3.65-3.60 (m, 1H), 3.59-3.51 (m, 2H), 2.41 (s, 1H), 2.19 (d, J=11.0 Hz, 1H), 1.36 (s, 9H), 0.87 (q, J=6.6, 5.8 Hz, 2H), 0.77 (s, 2H).

### Example 100: Synthesis of Compound 281

### Synthesis of Compound 281

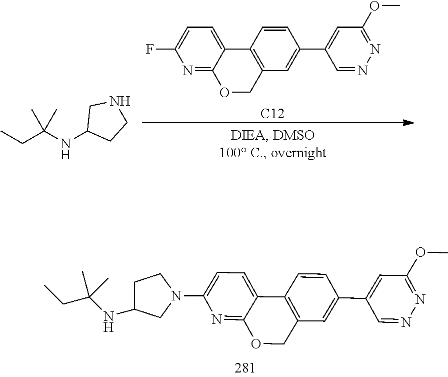

A mixture of N-(2-methylbutan-2-yl)pyrrolidin-3-amine (50 mg, 0.320 mmol, 1 equiv), 5-{3-fluoro-6H-isochromeno[3,4-b]pyridin-8-yl}-3-methoxypyridazine (98.96 mg, 0.320 mmol, 1 equiv), DIEA (124.06 mg, 0.960 mmol, 3 equiv), and DMSO (2 mL) was stirred for 3 h at 100° C. The resulting mixture was cooled to room temperature, then quenched with a mixture of water and ice (10 mL) at room temperature. A solid precipitated that was collected by filtration and washed with water (3×3 mL). The solid was purified by Prep-HPLC (Condition 10, Gradient 2) to afford 1-[8-(6-methoxypyridazin-4-yl)-6H-isochromeno[3,4-b]pyridin-3-yl]-N-(2-methylbutan-2-yl)pyrrolidin-3-amine (10.4 mg, 7%) as a solid. LCMS (ES, m/z): 446[M+H]⁺. ¹H NMR (400 MHz, DMSO-d₆) δ 9.33 (d, J=1.9 Hz, 1H), 8.08 (d, J=8.5 Hz, 1H), 7.90 (dd, J=8.3, 2.0 Hz, 1H), 7.82 (d, J=1.8 Hz, 1H), 7.77 (d, J=8.3 Hz, 1H), 7.50 (d, J=1.9 Hz, 1H), 6.22 (d, J=8.4 Hz, 1H), 5.27 (s, 2H), 4.08 (s, 3H), 3.67 (s, 1H), 3.53 (s, 1H), 3.46-3.38 (m, 1H), 3.30 (s, 1H), 2.97 (s, 1H), 2.13 (s, 1H), 1.72 (s, 1H), 1.38 (s, 2H), 1.01 (s, 6H), 0.83 (t, J=7.5 Hz, 3H).

### Example 101: Synthesis of Compound 316

### Synthesis of Intermediate C92

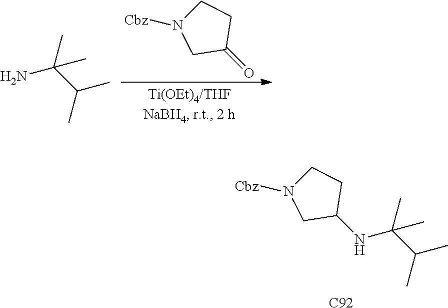

A solution of 2,3-dimethylbutan-2-amine (500 mg, 4.941 mmol, 1 equiv) in THE (2 mL)/Ti(OEt)₄ (1 mL) was treated with benzyl 3-oxopyrrolidine-1-carboxylate (812.46 mg, 3.706 mmol, 0.75 equiv) for 30 min at room temperature. To the reaction mixture was added NaBH₄ (560.76 mg, 14.823 mmol, 3 equiv) at 0° C. The resulting mixture was stirred for 2 h at room temperature, then quenched with water (5 mL) at 0° C. The aqueous layer was extracted with ethyl acetate (3×5 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (5:1) to afford benzyl 3-[(2,3-dimethylbutan-2-yl)amino]pyrrolidine-1-carboxylate (600 mg, 40%) as an oil.

### Synthesis of Intermediate C93

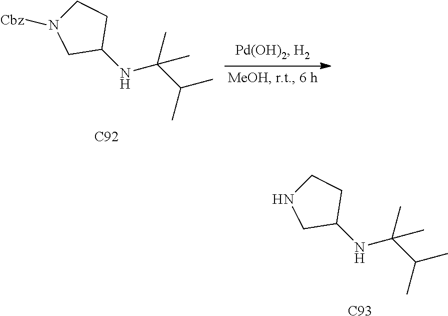 [SMILES: CC(C)C(C)(C)NC1CCN(C(=O)OCc2ccccc2)C1 ; "C92" ; CC(C)C(C)(C)NC1CCNC1 ; "C93"]

A mixture of benzyl 3-[(2,3-dimethylbutan-2-yl)amino]pyrrolidine-1-carboxylate (600 mg, 1.971 mmol, 1 equiv), Pd(OH)2/C (180 mg, 1.282 mmol, 0.65 equiv) and MeOH (15 mL, 370.509 mmol, 187.98 equiv) was stirred for 6 h at room temperature under hydrogen atmosphere. The resulting mixture was filtered, and the filter cake washed with ethyl acetate (3×10 mL). The filtrate was concentrated under reduced pressure to afford N-(2,3-dimethylbutan-2-yl)pyrrolidin-3-amine (240 mg, 72%) as an oil.

### Synthesis of Compound 316

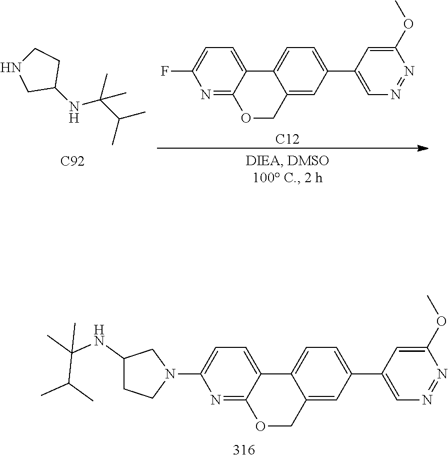

A mixture of 5-{3-fluoro-6H-isochromeno[3,4-b]pyridin-8-yl}-3-methoxypyridazine (50 mg, 0.162 mmol, 1 equiv), N-(2,3-dimethylbutan-2-yl)pyrrolidin-3-amine (41.29 mg, 0.243 mmol, 1.5 equiv), DIEA (62.68 mg, 0.486 mmol, 3 equiv) and DMSO (2 mL) was stirred for 2 h at 100° C., then quenched with water (3 mL) at room temperature. The aqueous layer was extracted with ethyl acetate (3×3 mL). The organic layers were combined, dried over anhydrous Na₂SO₄, and filtered. After filtration, the filtrate was concentrated under reduced pressure to give a residue. The residue was purified by Chiral-Prep-HPLC (Condition 2, Gradient 14) to afford N-(2,3-dimethylbutan-2-yl)-1-[8-(6-methoxypyridazin-4-yl)-6H-isochromeno[3,4-b]pyridin-3-yl]pyrrolidin-3-amine (14.7 mg, 20%) as a solid. LCMS (ES, m/z): 460[M+H]+. ¹H NMR (400 MHz, DMSO-d₆) δ 9.33 (d, J=1.9 Hz, 1H), 8.06 (d, J=8.5 Hz, 1H), 7.89 (dd, J=8.2, 2.0 Hz, 1H), 7.82 (d, J=2.0 Hz, 1H), 7.77 (d, J=8.2 Hz, 1H), 7.50 (d, J=1.9 Hz, 1H), 6.21 (d, J=8.5 Hz, 1H), 5.26 (s, 2H), 4.08 (s, 3H), 3.67 (s, 1H), 3.51-3.42 (m, 2H), 3.28 (dd, J=10.1, 6.9 Hz, 1H), 2.95 (dd, J=10.3, 7.5 Hz, 1H), 2.16-2.06 (m, 1H), 1.68 (ddd, J=26.6, 12.8, 7.8 Hz, 2H), 0.97 (d, J=7.2 Hz, 6H), 0.86 (d, J=6.8 Hz, 6H).

### Example 102: Synthesis of Compounds 290 and 291

### Synthesis of Intermediate C94

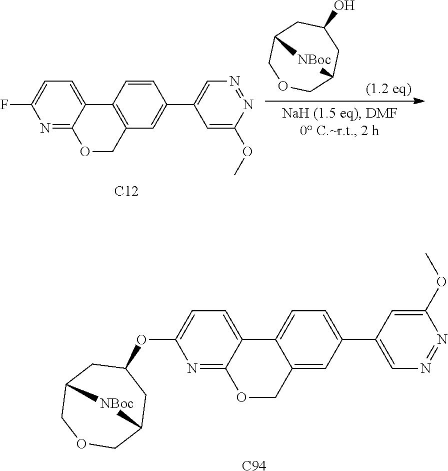

A solution of tert-butyl (exo)-7-hydroxy-3-oxa-9-azabicyclo[3.3.1]nonane-9-carboxylate (169.91 mg, 0.698 mmol, 1.2 equiv) in DMF (1.8 mL) was treated with NaH (20.95 mg, 0.873 mmol, 1.5 equiv). The reaction mixture was stirred for 0.5 h at 0° C. under nitrogen atmosphere. To the resulting mixture was added 5-{3-fluoro-6H-isochromeno[3,4-b]pyridin-8-yl}-3-methoxypyridazine (180 mg, 0.582 mmol, 1 equiv), and the reaction mixture was stirred for an additional 2 h at room temperature. The resulting mixture was poured into water, then extracted with ethyl acetate (1×10 mL). The organic layers were combined, washed with brine (3×10 mL), dried over anhydrous Na₂SO₄, and filtered. After filtration, the filtrate was concentrated under reduced pressure to afford tert-butyl (exo)-7-{[8-(6-methoxypyridazin-4-yl)-6H-isochromeno[3,4-b]pyridin-3-yl]oxy}-3-oxa-9-azabicyclo[3.3.1]nonane-9-carboxylate as a solid. LCMS (ES, m/z): 533[M+H]⁺.

### Synthesis of Compound 290

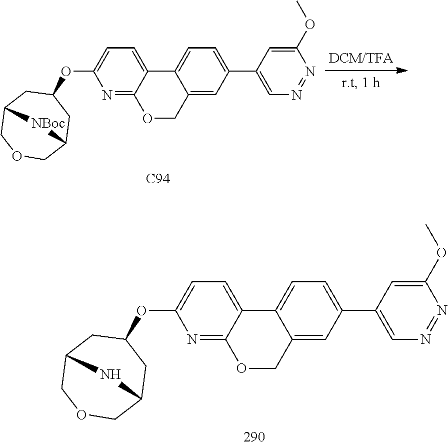

A mixture of tert-butyl (exo)-7-{[8-(6-methoxypyridazin-4-yl)-6H-isochromeno[3,4-b]pyridin-3-yl]oxy}-3-oxa-9-azabicyclo[3.3.1]nonane-9-carboxylate (195 mg, 0.366 mmol, 1 equiv) in DCM (1.5 mL) and TFA (0.5 mL) was stirred for 1 h at room temperature. The resulting mixture was concentrated under reduced pressure to give a residue. The residue was purified by Prep-HPLC (Gradient 16, Condition 1) to afford (exo)-7-{[8-(6-methoxypyridazin-4-yl)-6H-isochromeno[3,4-b]pyridin-3-yl]oxy}-3-oxa-9-azabicyclo[3.3.1]nonane (6 mg, 4%) as a solid. LCMS (ES, m/z): 433[M+H]⁺. ¹H NMR (400 MHz, DMSO-d₆) δ 9.35 (d, J=1.9 Hz, 1H), 8.30 (d, J=8.4 Hz, 1H), 7.95 (dd, J=8.2, 1.9 Hz, 1H), 7.93-7.86 (m, 2H), 7.54 (d, J=1.9 Hz, 1H), 6.52 (d, J=8.3 Hz, 1H), 6.00 (tt, J=11.2, 6.2 Hz, 1H), 5.38 (s, 2H), 4.08 (s, 3H), 3.75-3.64 (m, 4H), 2.96 (s, 2H), 2.22 (dd, J=12.1, 6.2 Hz, 2H), 1.74 (td, J=11.7, 4.8 Hz, 2H).

### Synthesis of Compound 291

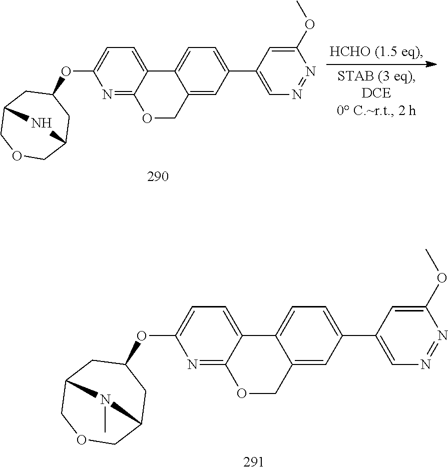 [SMILES: COc1cc(-c2ccc3c(c2)COc2nc(O[C@@H]4C[C@H]5COC[C@@H](C4)N5)ccc2-3)cnn1 ; "290" ; COc1cc(-c2ccc3c(c2)COc2nc(O[C@@H]4C[C@H]5COC[C@@H](C4)N5C)ccc2-3)cnn1 ; "291"]

A mixture of (exo)-7-{[8-(6-methoxypyridazin-4-yl)-6H-isochromeno[3,4-b]pyridin-3-yl]oxy}-3-oxa-9-azabicyclo[3.3.1]nonane (60 mg, 0.139 mmol, 1 equiv) and HCHO (6.25 mg, 0.209 mmol, 1.5 equiv) in DCE (0.6 mL) was stirred for 2 h at 0° C.˜r.t. under nitrogen atmosphere. The resulting mixture was poured into water, then extracted with CH₂Cl₂ (1×3 mL). The organic layer was dried over anhydrous Na₂SO₄, and filtered. After filtration, the filtrate was concentrated under reduced pressure to give a residue. The residue was purified by Prep-HPLC (Condition 11, Gradient 6) to afford (1R,5S,7r)-7-{[8-(6-methoxypyridazin-4-yl)-6H-isochromeno[3,4-b]pyridin-3-yl]oxy}-9-methyl-3-oxa-9-azabicyclo[3.3.1]nonane (1.4 mg, 2%) as a solid. LCMS (ES, m/z): 447[M+H]⁺. ¹H NMR (400 MHz, DMSO-d₆) δ 9.35 (d, J=1.9 Hz, 1H), 8.30 (d, J=8.4 Hz, 1H), 7.95-7.85 (m, 3H), 7.54 (d, J=2.0 Hz, 1H), 6.53 (d, J=8.3 Hz, 1H), 5.91 (p, J=8.6 Hz, 1H), 5.38 (s, 2H), 4.09 (s, 3H), 3.71 (q, J=11.1, 10.7 Hz, 4H), 2.75 (s, 2H), 2.44 (s, 3H), 1.95 (dd, J=8.7, 3.2 Hz, 4H).

### Example 103: Synthesis of Compound 225

### Synthesis of Compound 225

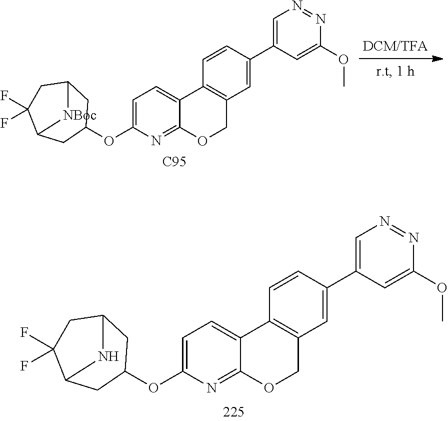 [SMILES: COc1cc(-c2ccc3c(c2)COc2nc(OC4CC5CC(F)(F)C(C4)N5C(=O)OC(C)(C)C)ccc2-3)cnn1 ; "C95" ; COc1cc(-c2ccc3c(c2)COc2nc(OC4CC5CC(F)(F)C(C4)N5)ccc2-3)cnn1]

A mixture of tert-butyl 6,6-difluoro-3-{[8-(6-methoxypyridazin-4-yl)-6H-isochromeno[3,4-b]pyridin-3-yl]oxy}-8-azabicyclo[3.2.1]octane-8-carboxylate (110 mg, 0.199 mmol, 1 equiv) in DCM (0.9 mL) and TFA (0.3 mL) was stirred for 1 h at room temperature. The resulting mixture was concentrated under reduced pressure to give a reside. The residue was purified by Prep-HPLC (Condition 11, Gradient 7) to afford (1R,3S,5S)-6,6-difluoro-3-{[8-(6-methoxypyridazin-4-yl)-6H-isochromeno[3,4-b]pyridin-3-yl]oxy}-8-azabicyclo[3.2.1]octane (44.6 mg, 50%) as a solid. LCMS (ES, m/z): 453[M+H]⁺. ¹H NMR (400 MHz, DMSO-d₆) δ 9.34 (d, J=1.9 Hz, 1H), 8.31 (d, J=8.4 Hz, 1H), 7.95-7.89 (dd, J=8.3, 1.9 Hz, 2H), 7.87 (d, 1H), 7.54 (d, J=1.9 Hz, 1H), 6.54 (d, J=8.3 Hz, 1H), 5.39 (s, 2H), 5.34 (dq, J=11.1, 6.2, 5.6 Hz, 1H), 4.08 (s, 3H), 3.60 (d, J=7.5 Hz, 1H), 3.45 (d, J=13.0 Hz, 1H), 2.89 (s, 1H), 2.35 (ddt, J=33.4, 12.6, 6.4 Hz, 2H), 2.25-2.11 (m, 2H), 1.72-1.52 (m, 2H).

### Example 104: Synthesis of Compounds 276-279, 294, 295, 298, and 299

### Synthesis of Intermediate C96

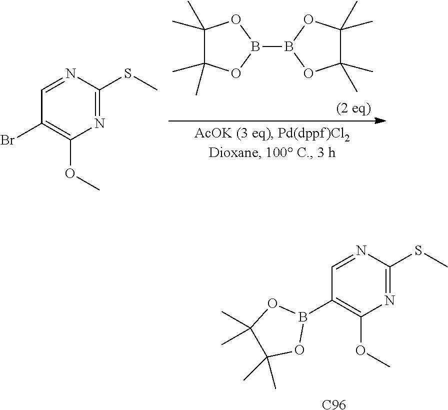

To a stirred solution of 5-bromo-4-methoxy-2-(methylsulfanyl)pyrimidine (500 mg, 2.127 mmol, 1 equiv) and bis(pinacolato)diboron (1.08 g, 4.254 mmol, 2 equiv) in dioxane (5 mL) were added Pd(dppf)Cl₂·CH₂Cl₂ (173 mg, 0.213 mmol, 0.1 equiv) and KOAc (626 mg, 6.381 mmol, 3 equiv) dropwise at room temperature. The resulting mixture was stirred for additional 3 h at 100° C. The mixture was allowed to cool down to room temperature. The resulting mixture was concentrated under vacuum. This resulted in 4-methoxy-2-(methylsulfanyl)-5-(4,4,5,5-tetramethyl-1,3,2-dioxaborolan-2-yl)pyrimidine (500 mg, 83%) as a solid. LCMS (ES, m/z): 283 [M+H]⁺.

### Synthesis of Intermediate C97

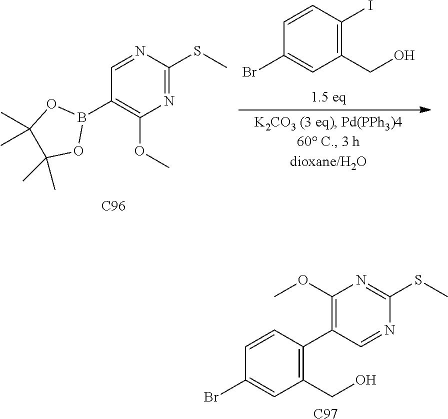

To a stirred mixture of 4-methoxy-2-(methylsulfanyl)-5-(4,4,5,5-tetramethyl-1,3,2-dioxaborolan-2-yl)pyrimidine (500 mg, 1.772 mmol, 1 equiv) and (5-bromo-2-iodophenyl)methanol (832 mg, 2.658 mmol, 1.5 equiv) in dioxane (10 mL) and water (2 mL) was added Pd(dppf)Cl₂·CH₂Cl₂ (144 mg, 0.177 mmol, 0.1 equiv) and K₃PO₄ (1.12 mg, 5.316 mmol, 3 equiv) in portions at room temperature under N₂ atmosphere. The resulting mixture was stirred for 3 h at 60° C., then cooled to room temperature and extracted with ethyl acetate (3×30 mL). The organic layers were combined, washed with water (3×30 mL), dried over anhydrous Na₂SO₄, and filtered. After filtration, the filtrate was concentrated under reduced pressure to give a residue. The residue was purified by silica gel column chromatography, eluted with PE/EA (1:1) to afford {5-bromo-2-[4-methoxy-2-(methylsulfanyl)pyrimidin-5-yl]phenyl}methanol (800 mg, 66%) as a solid. LCMS (ES, m/z): 341/343 [M+H]⁺.

### Synthesis of Intermediate C98

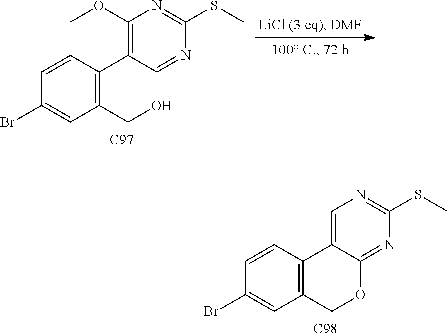 [SMILES: COc1nc(SC)ncc1-c1ccc(Br)cc1CO ; "C97" ; CSc1ncc2c(n1)OCc1cc(Br)ccc1-2 ; "C98"]

To a stirred solution of {5-bromo-2-[4-methoxy-2-(methylsulfanyl)pyrimidin-5-yl]phenyl}methanol (200 mg, 0.586 mmol, 1 equiv) in DMF (20 mL) was added LiCl (248 mg, 5.860 mmol, 10 equiv) in portions at room temperature. The resulting mixture was stirred for 72 h at 100° C., then cooled to room temperature and extracted with ethyl acetate (3×30 mL). The organic layers were combined, washed with water (3×30 mL), dried over anhydrous Na₂SO₄, and filtered. After filtration, the filtrate was concentrated under reduced pressure to give a residue. The residue was purified by silica gel column chromatography, eluted with PE/EA (1:5) to afford 8-bromo-3-(methylsulfanyl)-6H-isochromeno[3,4-d]pyrimidine (50 mg, 28%) as a solid. LCMS (ES, m/z): 309/311 [M+H]⁺.

### Synthesis of Intermediate C99

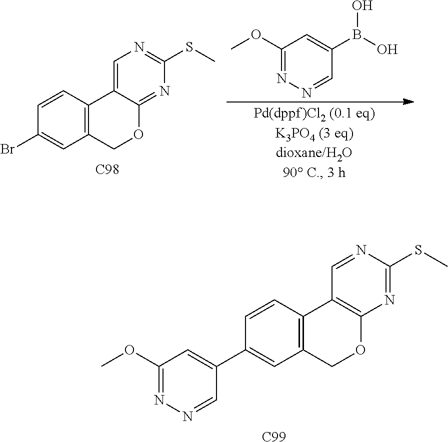

To a stirred mixture of 8-bromo-3-(methylsulfanyl)-6H-isochromeno[3,4-d]pyrimidine (100 mg, 0.323 mmol, 1 equiv) and 6-methoxypyridazin-4-ylboronic acid (75 mg, 0.485 mmol, 1.5 equiv) in dioxane (15 mL) and water (3 mL) was added Pd(dppf)Cl₂·CH₂Cl₂ (26 mg, 0.032 mmol, 0.1 equiv) and K₃PO₄ (206 mg, 0.969 mmol, 3 equiv) at room temperature under nitrogen atmosphere.

The resulting mixture was stirred for 3 h at 90° C., then cooled to room temperature and extracted with ethyl acetate (2×30 mL). The organic layers were combined, washed with water (3×30 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford 3-methoxy-5-[3-(methylsulfanyl)-6H-isochromeno[3,4-d]pyrimidin-8-yl]pyridazine (100 mg, 55%) as a solid. LCMS (ES, m/z): 339 [M+H]+.

### Synthesis of Intermediate C100

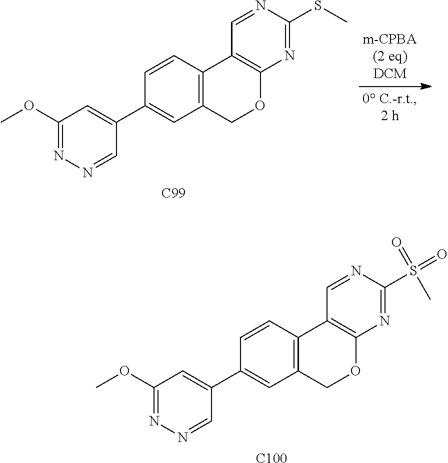

A mixture of 3-methoxy-5-[3-(methylsulfanyl)-6H-isochromeno[3,4-d]pyrimidin-8-yl]pyridazine (100 mg, 0.296 mmol, 1 equiv) and MCPBA (102 mg, 0.592 mmol, 2 equiv) in DCM (2 mL) was stirred for 2 h at room temperature, then concentrated under vacuum to afford product. LCMS (ES, m/z): 371 [M+H]+.

### Synthesis of Compounds 276 and 277

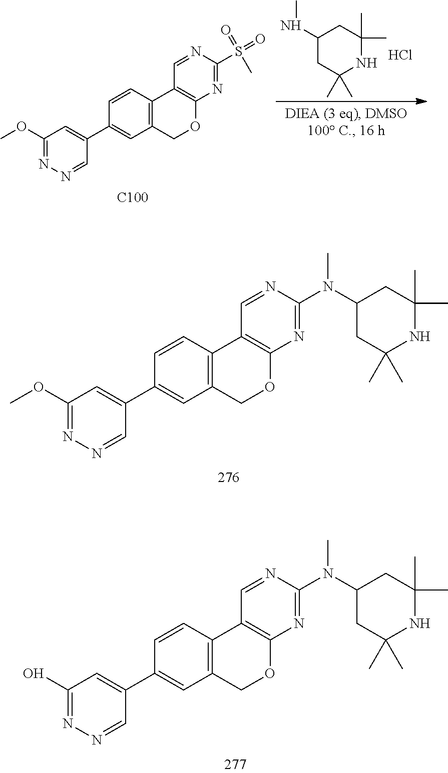

A mixture of 5-{3-methanesulfonyl-6H-isochromeno[3,4-d]pyrimidin-8-yl}-3-methoxypyridazine (45 mg, 0.121 mmol, 1 equiv) and N,2,2,6,6-pentamethylpiperidin-4-amine (41 mg, 0.242 mmol, 2 equiv) in DMSO (5 mL) was stirred for 16 h at 100° C., then cooled to room temperature. The resulting mixture was filtered. The filtrate was purified by Chiral-Prep-HPLC (Condition 14, Gradient 2), followed by Chiral-Prep-HPLC (Condition 2, Gradient 1) to afford N-[8-(6-methoxypyridazin-4-yl)-6H-isochromeno[3,4-d]pyrimidin-3-yl]-N,2,2,6,6-pentamethylpiperidin-4-amine (4.3 mg, 8%) (Compound 276) and 5-{3-[methyl(2,2,6,6-tetramethylpiperidin-4-yl)amino]-6H-isochromeno[3,4-d]pyrimidin-8-yl}pyridazin-3-ol (2.7 mg, 4%) solids. LCMS-Compound 276 (ES, m/z): 461 [M+H]⁺. LCMS-Compound 277 (ES, m/z): 447 [M+H]+.

| Compound Structure | Analysis data |
|---|---|
| 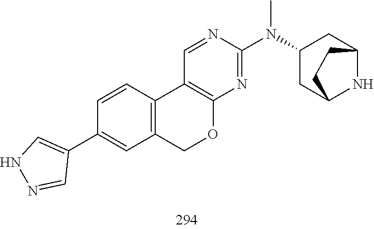 [SMILES: CN(c1ncc2c(n1)OCc1cc(-c3cn[nH]c3)ccc1-2)[C@@H]1C[C@H]2CC[C@@H](C1)N2] | LCMS (ESI, m/z): 389 [M + H]⁺. ¹H NMR (400 MHz, DMSO-d₆) δ 12.95 (s, 1H), 8.81 (s, 1H), 8.06 (s, 2H), 7.74 (d, J = 8.1 Hz, 1H), 7.58 (dd, J = 8.0, 1.9 Hz, 1H), 7.46 (d, J = 1.8 Hz, 1H), 5.35 (s, 2H), 5.06 (s, 1H), 3.47 (d, J = 4.4 Hz, 2H), 2.91 (s, 3H), 1.78 − 1.59 (m, 5H), 1.50 − 1.40 (m, 3H). |
| 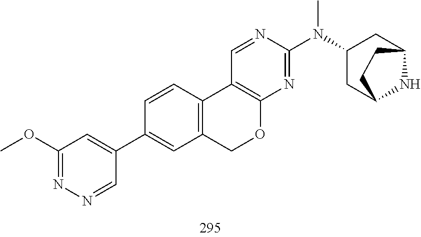 [SMILES: COc1cc(-c2ccc3c(c2)COc2nc(N(C)[C@@H]4C[C@H]5CC[C@@H](C4)N5)ncc2-3)cnn1] | LCMS (ESI, m/z): 431 [M + H]⁺. ¹H NMR (400 MHz, DMSO-d₆) δ 9.33 (d, J = 1.9 Hz, 1H), 8.92 (s, 1H), 7.92 (d, J = 1.2 Hz, 2H), 7.83 (s, 1H), 7.51 (d, J = 1.9 Hz, 1H), 5.42 (s, 2H), 5.10 (s, 1H), 4.08 (s, 3H), 3.49 (s, 2H), 2.93 (s, 3H), 1.80 − 1.69 (m, 4H), 1.65 (t, J = 6.6 Hz, 2H), 1.46 (ddd, J = 12.5, 6.0, 2.6 Hz, 2H). |
| 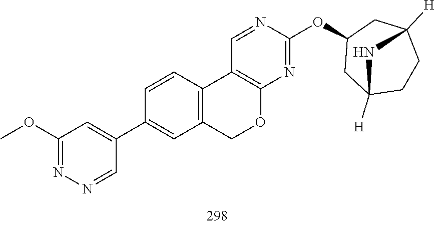 [SMILES: COc1cc(-c2ccc3c(c2)COc2nc(O[C@@H]4C[C@H]5CC[C@@H](C4)N5)ncc2-3)cnn1] | LCMS (ESI, m/z): 418 [M + H]⁺. ¹H NMR (400 MHz, DMSO-d₆) δ 9.35 (d, J = 1.9 Hz, 1H), 9.11 (s, 1H), 8.07 (d, J = 8.3 Hz, 1H), 7.98 (dd, J = 8.2, 1.9 Hz, 1H), 7.89 (d, J = 1.9 Hz, 1H), 7.56 (d, J = 2.0 Hz, 1H), 5.55 (s, 2H), 5.23 (tt, J = 10.9, 5.9 Hz, 1H), 4.09 (s, 3H), 3.52 (s, 2H), 2.07 − 2.04 (m, 2H), 1.73 (d, J = 4.1 Hz, 2H), 1.72 (s, 2H), 1.69 − 1.52 (m, 2H). |
| 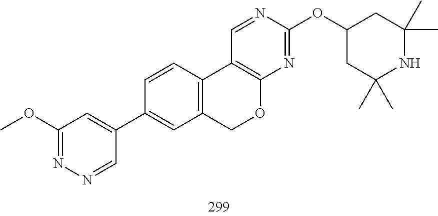 | LCMS (ESI, m/z): 448 [M + H]⁺. ¹H NMR (400 MHz, DMSO-d₆) δ 9.35 (d, J = 1.9 Hz, 1H), 9.13 (s, 1H), 8.08 (d, J = 8.2 Hz, 1H), 7.98 (dd, J = 8.1, 2.0 Hz, 1H), 7.89 (d, J = 1.9 Hz, 1H), 7.56 (d, J = 1.9 Hz, 1H), 5.55 (s, 2H), 5.39 (tt, J = 11.3, 4.1 Hz, 1H), 4.09 (s, 3H), 1.98 (dd, J = 11.9, 4.1 Hz, 2H), 1.24 (d, J = 11.5 Hz, 2H), 1.20 (s, 6H), 1.09 (s, 6H). |
| 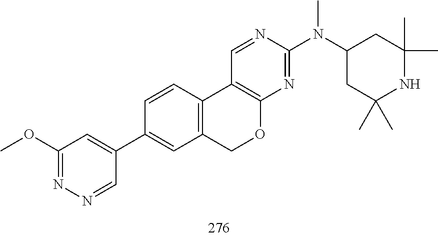 | LCMS (ESI, m/z): 461 [M + H]⁺. ¹H NMR (400 MHz, DMSO-d₆) δ 9.33 (d, J = 1.9 Hz, 1H), 8.94 (s, 1H), 7.98 − 7.88 (m, 2H), 7.83 (s, 1H), 7.52 (d, J = 1.9 Hz, 1H), 5.42 (s, 2H), 5.24 (s, 1H), 4.08 (s, 3H), 2.96 (s, 3H), 1.50 − 1.32 (m, 5H), 1.21 (s, 6H), 1.07 (s, 6H). |
| 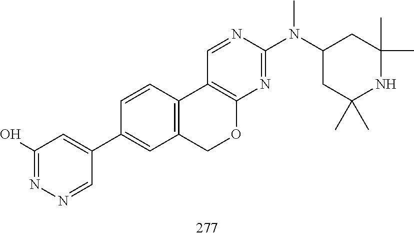 [SMILES: CN(c1ncc2c(n1)OCc1cc(-c3cnnc(O)c3)ccc1-2)C1CC(C)(C)NC(C)(C)C1] | LCMS (ESI, m/z): 447 [M + H]⁺ ¹H NMR (400 MHz, DMSO-d₆) δ 13.12 (s, 1H), 8.89 (s, 1H), 8.35 (d, J = 2.1 Hz, 1H), 7.91 (d, J = 8.3 Hz, 1H), 7.81 (dd, J = 8.2, 1.9 Hz, 1H), 7.72 (d, J = 1.9 Hz, 1H), 7.15 (d, J = 2.1 Hz, 1H), 5.42 (s, 2H), 5.22 (s, 1H), 2.96 (s, 3H), 1.49 − 1.36 (m, 4H), 1.23 (s, 6H), 1.10 (s, 6H). |
| 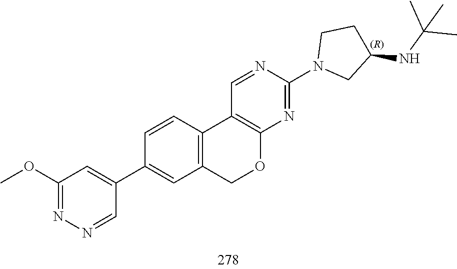 | LCMS (ESI, m/z): 433 [M + H]⁺. ¹H NMR (400 MHz, DMSO-d₆) δ 9.33 (d, J = 2.0 Hz, 1H), 8.89 (s, 1H), 7.92 (d, J = 1.3 Hz, 2H), 7.83 (t, J = 1.2 Hz, 1H), 7.51 (d, J = 2.0 Hz, 1H), 5.40 (s, 2H), 4.08 (s, 3H), 3.82- 3.58 (m, 3H), 3.49 − 3.41 (m, 1H), 3.10 − 2.99 (m, 1H), 2.11 (q, J = 4.7, 4.2 Hz, 1H), 1.76 − 1.62 (m, 1H), 1.07 (s, 9H). |
| 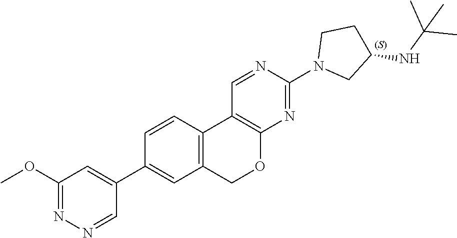 | LCMS (ESI, m/z): 433 [M + H]⁺. ¹H NMR (400 MHz, DMSO-d₆) δ 9.33 (d, J = 1.9 Hz, 1H), 8.89 (s, 1H), 7.92 (d, J = 1.3 Hz, 2H), 7.83 (t, J = 1.2 Hz, 1H), 7.52 (d, J = 1.9 Hz, 1H), 5.40 (s, 2H), 4.08 (s, 3H), 3.82- 3.58 (s, 3H), 3.46 (q, J = 7.1 Hz, 1H), 3.04 (t, J = 13.1 Hz, 1H), 2.10 (dq, J = 10.6, 5.9, 5.4 Hz, 1H), 1.75 - 1.61 (m, 1H), 1.06 (s, 9H). |
| 279 | |

### Example 105: Synthesis of Compounds 303 and 304

### Synthesis of Intermediate C101

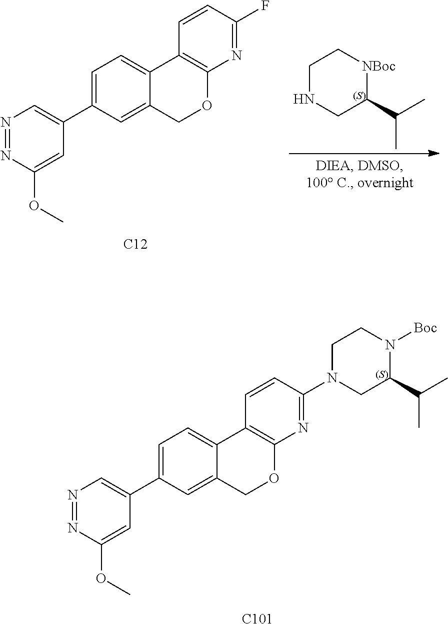

A mixture of 5-{3-fluoro-6H-isochromeno[3,4-b] pyridin-8-yl}-3-methoxypyridazine (100 mg, 0.323 mmol, 1.0 equiv) and tert-butyl (2S)-2-isopropylpiperazine-1-carboxylate (81.21 mg, 0.355 mmol, 1.1 equiv) in DMSO (1.0 mL) was treated with DIEA (167.15 mg, 1.292 mmol, 4.0 equiv). The reaction mixture was stirred overnight at 100° C. under nitrogen atmosphere, then extracted with ethyl acetate (3×50 mL). The organic layers were combined, washed with brine (3×30 mL), dried over anhydrous Na₂SO₄, and filtered. After filtration, the filtrate was concentrated under reduced pressure to afford tert-butyl (2S)-2-isopropyl-4-[8-(6-methoxypyridazin-4-yl)-6H-isochromeno[3,4-b]pyridin-3-yl] piperazine-1-carboxylate (100 mg) as a solid.

### Synthesis of Compound 303

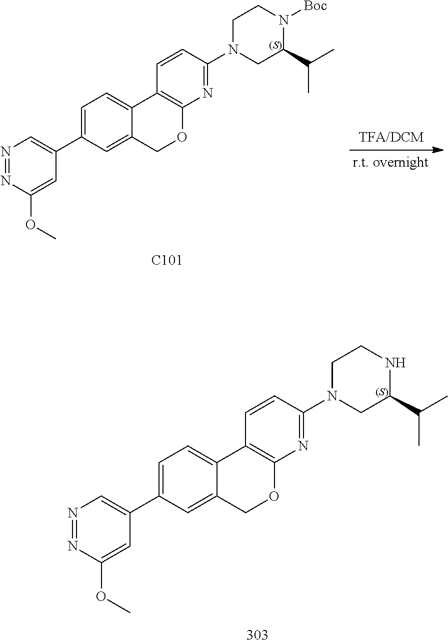

A solution of tert-butyl (2S)-2-isopropyl-4-[8-(6-methoxypyridazin-4-yl)-6H-isochromeno[3,4-b] pyridin-3-yl] piperazine-1-carboxylate (100 mg, 0.193 mmol, 1.0 equiv) in DCM (1.0 mL) was treated with TFA (100 uL) overnight at room temperature under nitrogen atmosphere. The reaction mixture was basified to pH 6 with saturated Na₂CO₃ (aq.), then extracted with CH₂C12 (3×50 mL). The organic layers were combined, washed with brine (3×50 mL), dried over anhydrous Na₂SO₄, and filtered. After filtration, the filtrate was concentrated under reduced pressure to give a residue. The residue was purified by Chiral-Prep-HPLC (Condition 11, Gradient 6) to afford 5-{3-[(3S)-3-isopropylpiperazin-1-yl]-6H-isochromeno[3,4-b] pyridin-8-yl}-3-methoxypyridazine (18.7 mg, 23%) as a solid.

| Compound Structure | Analysis data |
|---|---|
| 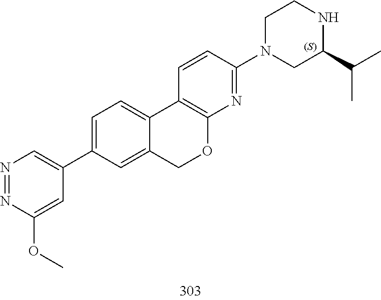 | LCMS (ES, m/z): 418 [M + H]⁺. ¹H NMR (400 MHz, DMSO-d₆) δ 9.33 (d, J = 2.0 Hz, 1H), 8.09 (d, J = 8.8 Hz, 1H), 7.90 (dd, J = 8.2, 2.0 Hz, 1H), 7.84 − 7.77 (m, 2H), 7.51 (d, J = 2.0 Hz, 1H), 6.58 (d, J = 8.6 Hz, 1H), 5.29 (s, 2H), 4.24 − 4.16 (m, 1H), 4.08 (s, 4H), 3.01 − 2.94 (q, 1H), 2.98 (dt, J = 11.8, 2.6 Hz, 1H), 2.77 (td, J = 12.0, 3.0 Hz, 1H), 2.63-2.59 (td, J = 11.7, 3.1 Hz, 1H), 2.29 (ddd, J = 9.6, 5.9, 2.8 Hz, 2H), 1.61 (dq, J = 13.4, 6.7 Hz, 1H), 0.95 (dd, J = 6.8, 1.1 Hz, 6H). |
| 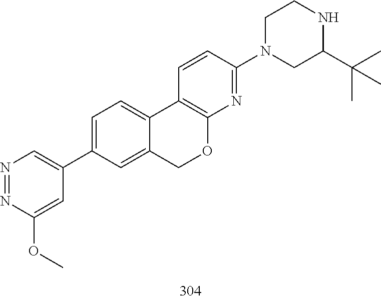 [SMILES: COc1cc(-c2ccc3c(c2)COc2nc(N4CCNC(C(C)(C)C)C4)ccc2-3)cnn1] | LCMS (ES, m/z): 432 [M + H]⁺. ¹H NMR (400 MHz, DMSO-d₆) δ 9.33 (d, J = 1.9 Hz, 1H), 8.09 (d, J = 8.7 Hz, 1H), 7.90 (dd, J = 8.1, 2.0 Hz, 1H), 7.84 − 7.77 (m, 2H), 7.51 (d, J = 2.0 Hz, 1H), 6.56 (d, J = 8.6 Hz, 1H), 5.29 (s, 2H), 4.30 (d, J = 12.3 Hz, 1H), 4.08 (s, 4H), 3.01 (d, J = 11.5 Hz, 1H), 2.74 (td, J = 12.0, 2.8 Hz, 1H), 2.69 − 2.59 (m, 1H), 2.54 (s, 1H), 2.23 (d, J = 10.6 Hz, 2H), 0.94 (s, 9H). |

### Example 106: Synthesis of Compounds 317 and 318

### Synthesis of Compounds 317 and 318

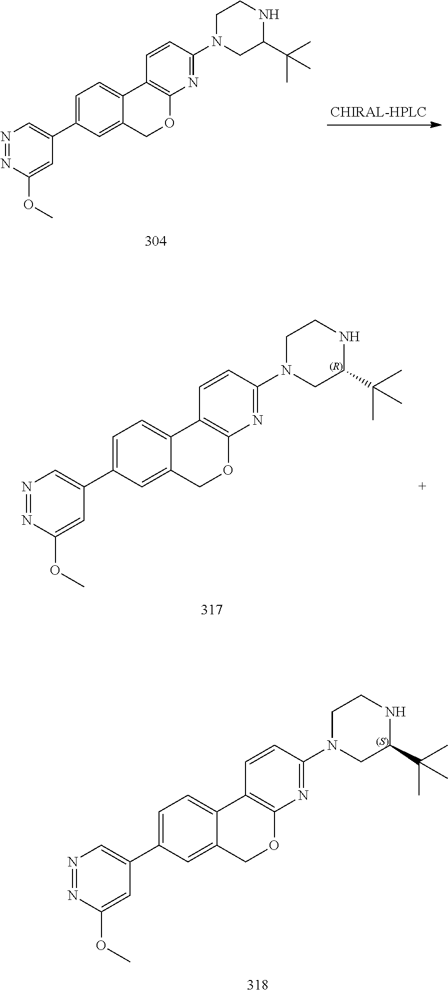

5-[3-(3-tert-butylpiperazin-1-yl)-6H-isochromeno[3,4-b]pyridin-8-yl]-3-methoxypyridazine (20 mg) was purified by CHIRAL-HPLC (Condition 3, Gradient 1) to afford 5-{3-[(3R)-3-tert-butylpiperazin-1-yl]-6H-isochromeno [3,4-b]pyridin-8-yl}-3-methoxypyridazine (4.1 mg, 20%) and 5-{3-[(3S)-3-tert-butylpiperazin-1-yl]-6H-isochromeno[3,4-b]pyridin-8-yl}-3-methoxypyridazine (3.6 mg, 18%) as solids. LCMS-Compound 317: (ES, m/z): 432 [M+H]⁺. ¹H NMR-Compound 317: (400 MHz, DMSO-d₆) δ 9.34 (d, J=2.0 Hz, 1H), 8.10 (d, J=8.7 Hz, 1H), 7.91 (dd, J=8.2, 2.0 Hz, 1H), 7.85-7.77 (m, 2H), 7.51 (d, J=2.0 Hz, 1H), 6.57 (d, J=8.7 Hz, 1H), 5.29 (s, 2H), 4.31 (d, J=12.4 Hz, 1H), 4.13 (d, J=12.1 Hz, 1H), 4.08 (s, 3H), 3.02 (d, J=11.5 Hz, 1H), 2.75 (t, J=11.8 Hz, 1H), 2.63 (d, J=11.5 Hz, 1H), 2.55 (s, 1H), 2.25 (d, J=10.6 Hz, 1H), 0.95 (s, 9H). LCMS-Compound 318: (ES, m/z): 432 [M+H]⁺. ¹H NMR-Compound 318: (400 MHz, DMSO-d₆) δ 9.34 (d, J=2.0 Hz, 1H), 8.10 (d, J=8.7 Hz, 1H), 7.91 (dd, J=8.2, 2.0 Hz, 1H), 7.85-7.77 (m, 2H), 7.51 (d, J=2.0 Hz, 1H), 6.57 (d, J=8.7 Hz, 1H), 5.29 (s, 2H), 4.31 (d, J=12.4 Hz, 1H), 4.13 (d, J=12.1 Hz, 1H), 4.08 (s, 3H), 3.02 (d, J=11.5 Hz, 1H), 2.75 (t, J=11.8 Hz, 1H), 2.63 (d, J=11.5 Hz, 1H), 2.55 (s, 1H), 2.25 (d, J=10.6 Hz, 1H), 0.95 (s, 9H).

### Example 107: Synthesis of Compound 300

### Synthesis of Intermediate C102

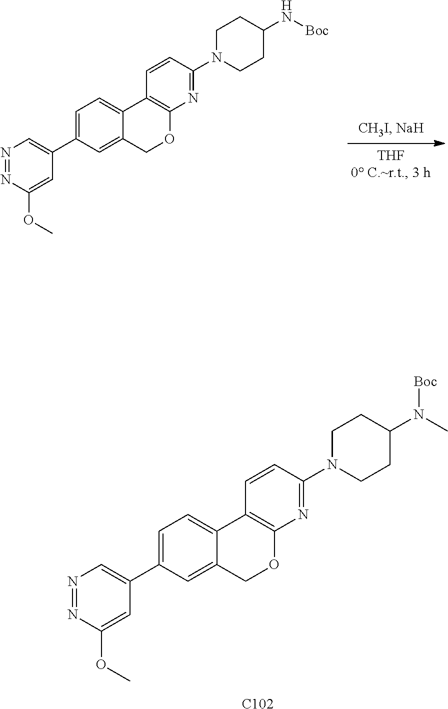 [SMILES: COc1cc(-c2ccc3c(c2)COc2nc(N4CCC(NC(=O)OC(C)(C)C)CC4)ccc2-3)cnn1 ; COc1cc(-c2ccc3c(c2)COc2nc(N4CCC(N(C)C(=O)OC(C)(C)C)CC4)ccc2-3)cnn1 ; "C102"]

To a solution of tert-butyl N-{1-[8-(6-methoxypyridazin-4-yl)-6H-isochromeno[3,4-b] pyridin-3-yl] piperidin-4-yl} carbamate (100 mg, 0.204 mmol, 1.0 equiv) in THE (2.0 mL) was added NaH (6.37 mg, 0.265 mmol, 1.3 equiv) at 0° C. under nitrogen atmosphere, then stirred for 30 min. To the reaction mixture was added CH₃I (43.49 mg, 0.306 mmol, 1.5 equiv). The resulting mixture was allowed to warm to room temperature, then stirred for 3 h. The reaction mixture was quenched with water (5 mL) at room temperature, then extracted with ethyl acetate (3×50 mL). The organic layers were combined, washed with brine (3×30 mL), dried over anhydrous Na₂SO₄, and filtered. After filtration, the filtrate was concentrated under reduced pressure to afford tert-butyl N-{1-[8-(6-methoxypyridazin-4-yl)-6H-isochromeno[3,4-b]pyridin-3-yl]piperidin-4-yl}-N-methylcarbamate (100 mg) as a solid.

### Synthesis of Compound 300

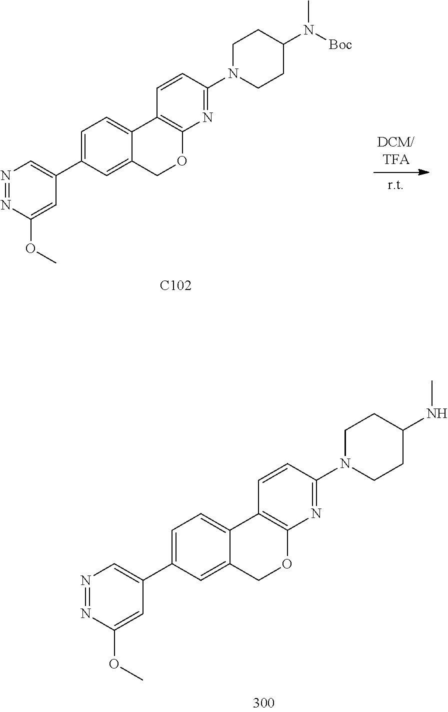 [SMILES: COc1cc(-c2ccc3c(c2)COc2nc(N4CCC(N(C)C(=O)OC(C)(C)C)CC4)ccc2-3)cnn1 ; "C102" ; CNC1CCN(c2ccc3c(n2)OCc2cc(-c4cnnc(OC)c4)ccc2-3)CC1]

A solution of tert-butyl N-{1-[8-(6-methoxypyridazin-4-yl)-6H-isochromeno[3,4-b] pyridin-3-yl]piperidin-4-yl}-N-methyl carbamate (85 mg, 0.169 mmol, 1.0 equiv) in DCM (1.0 mL) was treated with TFA (100 uL). The reaction mixture was stirred overnight at room temperature under nitrogen atmosphere, then basified to pH 6 with saturated Na₂CO₃ (aq.) and extracted with CH₂Cl₂ (3×50 mL). The organic layers were combined, washed with brine (3×30 mL), dried over anhydrous Na₂SO₄, and filtered. After filtration, the filtrate was concentrated under reduced pressure to give a residue. The residue was purified by Chiral-Prep-HPLC (Condition 16, Gradient 2) to afford 1-[8-(6-methoxypyridazin-4-yl)-6H-isochromeno[3,4-b] pyridin-3-yl]-N-methylpiperidin-4-amine (8.3 mg, 12%) as a solid. LCMS (ES, m/z): 404 [M+H]⁺. ¹H NMR (400 MHz, DMSO-d6) δ 9.33 (d, J=2.0 Hz, 1H), 8.09 (d, J=8.7 Hz, 1H), 7.90 (dd, J=8.1, 2.0 Hz, 1H), 7.84-7.77 (m, 2H), 7.51 (d, J=1.9 Hz, 1H), 6.61 (d, J=8.7 Hz, 1H), 5.28 (s, 2H), 4.18 (d, J=13.2 Hz, 2H), 4.08 (s, 3H), 3.04-2.94 (m, 2H), 2.56 (s, 1H), 2.30 (s, 3H), 1.85 (d, J=12.4 Hz, 2H), 1.19 (q, J=13.2, 11.8 Hz, 2H).

### Example 108: Synthesis of Compound 301

### Synthesis of Compound 301

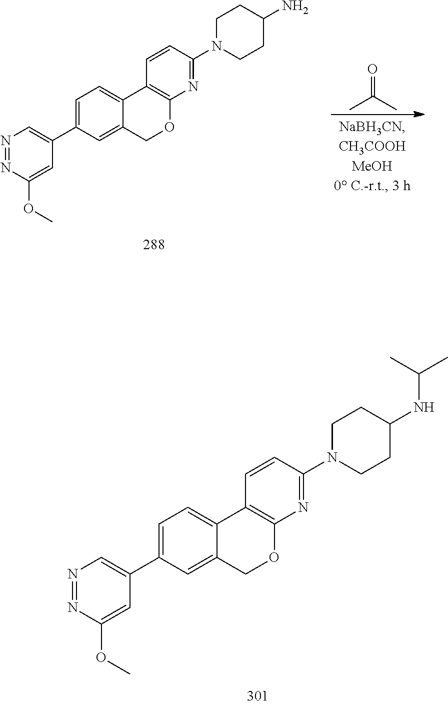

A solution of 1-[8-(6-methoxypyridazin-4-yl)-6H-isochromeno[3,4-b] pyridin-3-yl] piperidin-4-amine (100 mg, 0.257 mmol, 1.0 equiv) in methanol (2.0 mL) was treated with acetone (29.83 mg, 0.514 mmol, 2.0 equiv) and CH₃COOH (77.10 mg, 1.285 mmol, 5.0 equiv). The resulting mixture was stirred for 1 h at room temperature under nitrogen atmosphere. To the reaction mixture was added NaBH₃CN (32.27 mg, 0.514 mmol, 2.0 equiv) at 0° C. under nitrogen atmosphere. The resulting mixture was allowed to warm to room temperature and stirred for 3 hours, then quenched with water (3.0 mL) at room temperature. The resulting mixture was extracted with ethyl acetate (3×50 mL). The organic layers were combined, washed with brine (3×30 mL), dried over anhydrous Na₂SO₄, and filtered. After filtration, the filtrate was concentrated under reduced pressure to give a residue. The residue was purified by Chiral-Prep-HPLC (Condition 11, Gradient 8) to afford N-isopropyl-1-[8-(6-methoxypyridazin-4-yl)-6H-isochromeno[3,4-b] pyridin-3-yl] piperidin-4-amine (16.7 mg, 15%) as a solid. LCMS (ES, m/z): 432 [M+H]+. 1H NMR (400 MHz, DMSO-d6) δ 9.33 (d, J=1.9 Hz, 1H), 8.09 (d, J=8.7 Hz, 1H), 7.90 (dd, J=8.1, 2.0 Hz, 1H), 7.84-7.77 (m, 2H), 7.51 (d, J=1.9 Hz, 1H), 6.60 (d, J=8.7 Hz, 1H), 5.28 (s, 2H), 4.22 (dt, J=13.5, 3.8 Hz, 2H), 4.08 (s, 3H), 2.94 (td, J=12.7, 11.0, 4.7 Hz, 3H), 2.78 (d, J=10.8 Hz, 1H), 1.84 (dd, J=13.2, 3.7 Hz, 2H), 1.17 (q, J=11.6, 9.7 Hz, 2H), 0.98 (d, J=6.2 Hz, 6H).

### Example 109: Synthesis of Compound 302

### Synthesis of Compound 302

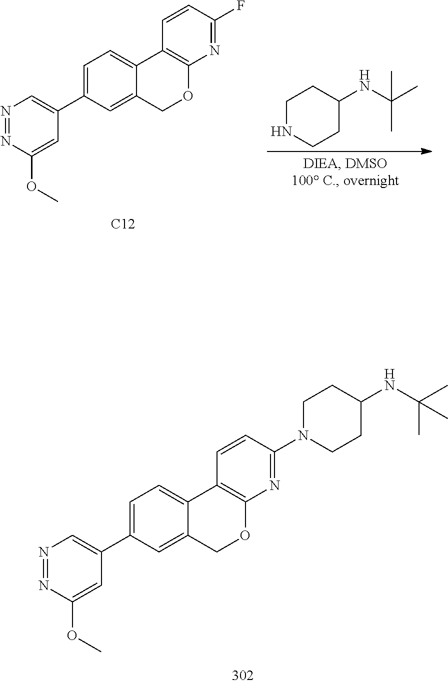 [SMILES: COc1cc(-c2ccc3c(c2)COc2nc(F)ccc2-3)cnn1 ; CC(C)(C)NC1CCNCC1 ; "C12" ; COc1cc(-c2ccc3c(c2)COc2nc(N4CCC(NC(C)(C)C)CC4)ccc2-3)cnn1]

A mixture of 5-{3-fluoro-6H-isochromeno[3,4-b] pyridin-8-yl}-3-methoxypyridazine (80 mg, 0.259 mmol, 1.0 equiv) and N-tert-butylpiperidin-4-amine (60.63 mg, 0.389 mmol, 1.5 equiv) in DMSO (1 mL) was treated with DIEA (133.72 mg, 1.036 mmol, 4.0 equiv). The reaction mixture was stirred overnight at 100° C. under nitrogen atmosphere, then extracted with ethyl acetate (3×20 mL). The organic layers were combined, washed with brine (3×20 mL), dried over anhydrous Na₂SO₄, and filtered. After filtration, the filtrate was concentrated under reduced pressure to give a residue. The residue was purified by Chiral-Prep-HPLC (Condition 12, Gradient 2) to afford 5-{3-[4-(tert-butylamino) piperidin-1-yl]-6H-isochromeno[3,4-b] pyridin-8-yl} pyridazin-3-ol (19.1 mg, 17%) as a solid. LCMS (ES, m/z): 446 [M+H]⁺. ¹H NMR (400 MHz, DMSO-d₆) δ 9.33 (d, J=1.9 Hz, 1H), 8.08 (d, J=8.7 Hz, 1H), 7.90 (dd, J=8.1, 2.0 Hz, 1H), 7.84-7.76 (m, 2H), 7.51 (d, J=1.9 Hz, 1H), 6.59 (d, J=8.7 Hz, 1H), 5.28 (s, 2H), 4.18 (d, J=13.2 Hz, 2H), 4.08 (s, 3H), 2.98 (ddd, J=13.7, 11.6, 2.7 Hz, 2H), 2.79 (s, 1H), 1.75 (d, J 12.5 Hz, 2H), 1.22 (d, J=11.6 Hz, 2H), 1.06 (s, 9H)

### Example 110: Synthesis of Compound 319

### Synthesis of Compound 319

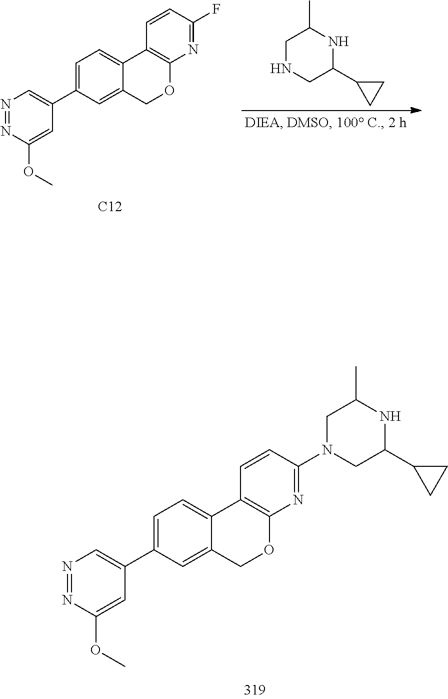

A mixture of 5-{3-fluoro-6H-isochromeno[3,4-b] pyridin-8-yl}-3-methoxypyridazine (50 mg, 0.162 mmol, 1.0 equiv) and (2S,6R)-2-cyclopropyl-6-methylpiperazine (45.34 mg, 0.324 mmol, 2.0 equiv) in DMSO (0.5 mL) was treated with DIEA (83.57 mg, 0.648 mmol, 4.0 equiv). The reaction mixture was stirred overnight at 90° C. under nitrogen atmosphere, then extracted with ethyl acetate (3×50 mL). The organic layers were combined, washed with brine (3×50 mL), dried over anhydrous Na₂SO₄, and filtered. After filtration, the filtrate was concentrated under reduced pressure to give a residue. The residue was purified by Chiral-Prep-HPLC (Condition 11, Gradient 9) to afford 5-{3-[(3S,5R)-3-cyclopropyl-5-methylpiperazin-1-yl]-6H-isochromeno[3,4-b]pyridin-8-yl}-3-methoxypyridazine (2.2 mg) as a solid. LCMS (ES, m/z): 430 [M+H]+. 1H NMR (400 MHz, DMSO-d₆) δ 9.33 (d, J=1.9 Hz, 1H), 8.09 (d, J=8.7 Hz, 1H), 7.90 (dd, J=8.2, 2.0 Hz, 1H), 7.84-7.77 (m, 2H), 7.51 (d, J=1.9 Hz, 1H), 6.60 (d, J 8.7 Hz, 1H), 5.29 (s, 2H), 4.26 (d, J=12.1 Hz, 1H), 4.13 (d, J=12.1 Hz, 1H), 4.08 (s, 3H), 2.64 (d, J=5.7 Hz, 1H), 2.38-2.25 (m, 2H), 1.88 (t, J=9.4 Hz, 1H), 1.05 (d, J=6.1 Hz, 3H), 0.73 (qt, J=8.4, 4.8 Hz, 1H), 0.43 (dh, J=8.0, 3.8, 2.4 Hz, 2H), 0.27 (td, J=4.8, 2.6 Hz, 2H).

### Example 111: Synthesis of Compounds 307

### Synthesis of Intermediate C103

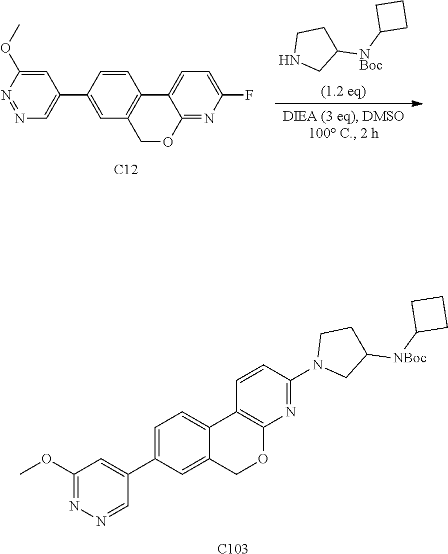 [SMILES: COc1cc(-c2ccc3c(c2)COc2nc(F)ccc2-3)cnn1 ; CC(C)(C)OC(=O)N(C1CCC1)C1CCNC1 ; "C12" ; COc1cc(-c2ccc3c(c2)COc2nc(N4CCC(N(C(=O)OC(C)(C)C)C5CCC5)C4)ccc2-3)cnn1 ; "C103"]

A mixture of 5-{3-fluoro-6H-isochromeno[3,4-b]pyridin-8-yl}-3-methoxypyridazine (90 mg, 0.291 mmol, 1 equiv), tert-butyl N-cyclobutyl-N-(pyrrolidin-3-yl)carbamate (84 mg, 0.349 mmol, 1.2 equiv), and DIEA (113 mg, 0.873 mmol, 3 equiv) in DMSO (0.9 mL) was stirred for 2 h at 100° C., then cooled to room temperature. The resulting mixture was poured into water and extracted with ethyl acetate (1×20 mL). The organic layer was dried over anhydrous Na₂SO₄ and filtered. After filtration, the filtrate was concentrated under reduced pressure to afford tert-butyl N-cyclobutyl-N-{1-[8-(6-methoxypyridazin-4-yl)-6H-isochromeno[3,4-b]pyridin-3-yl]pyrrolidin-3-yl}carbamate (139 mg, 90%) as a solid. LCMS (ES, m/z): 530 [M+H]⁺.

### Synthesis of Compound 280

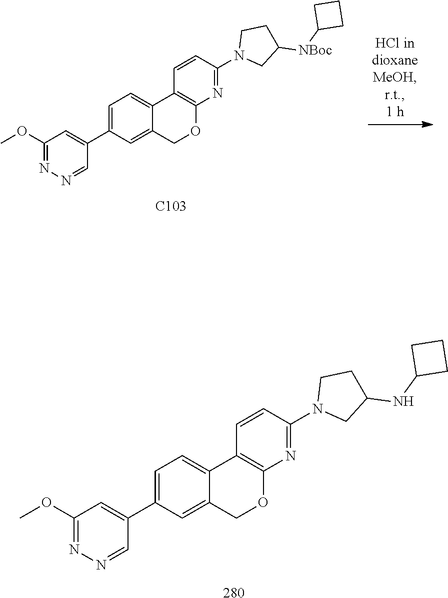 [SMILES: COc1cc(-c2ccc3c(c2)COc2nc(N4CCC(N(C(=O)OC(C)(C)C)C5CCC5)C4)ccc2-3)cnn1 ; "C103" ; COc1cc(-c2ccc3c(c2)COc2nc(N4CCC(NC5CCC5)C4)ccc2-3)cnn1 ; "280"]

A mixture of tert-butyl N-cyclobutyl-N-{1-[8-(6-methoxypyridazin-4-yl)-6H-isochromeno[3,4-b]pyridin-3-yl]pyrrolidin-3-yl}carbamate (125 mg, 0.236 mmol, 1 equiv) in methanol (1.25 mL) and HCl (gas) in 1,4-dioxane (1.25 mL, 41.141 mmol, 174.32 equiv) was stirred for 1 h at room temperature. The resulting mixture was concentrated under reduced pressure, the basified to pH 9 with saturated NaHCO₃ (aq.) and extracted with ethyl acetate (1×20 mL). The organic layer was dried over anhydrous Na₂SO₄ and filtered. After filtration, the filtrate was concentrated under reduced pressure to afford N-cyclobutyl-1-[8-(6-methoxypyridazin-4-yl)-6H-isochromeno[3,4-b]pyridin-3-yl]pyrrolidin-3-amine (100 mg, 99%) as a solid. LCMS (ES, m/z): 430 [M+H]+.

### Synthesis of Compound 307

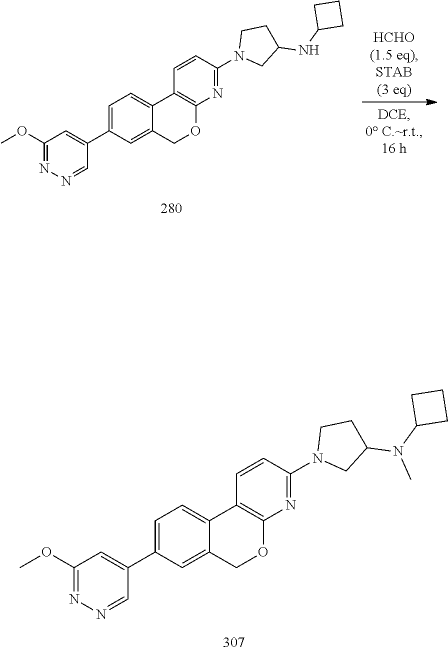 [SMILES: COc1cc(-c2ccc3c(c2)COc2nc(N4CCC(NC5CCC5)C4)ccc2-3)cnn1 ; "280" ; COc1cc(-c2ccc3c(c2)COc2nc(N4CCC(N(C)C5CCC5)C4)ccc2-3)cnn1 ; "307"]

To a mixture of N-cyclobutyl-1-[8-(6-methoxypyridazin-4-yl)-6H-isochromeno[3,4-b]pyridin-3-yl]pyrrolidin-3-amine (100 mg, 0.233 mmol, 1 equiv) and HCHO (11 mg, 0.350 mmol, 1.5 equiv) at 0° C. was added STAB (148 mg, 0.699 mmol, 3 equiv) in DCE (1 mL). The reaction mixture was stirred for 16 h at room temperature. The resulting mixture was poured into water and extracted with CH₂Cl₂ (1×30 mL). The organic layer was dried over anhydrous Na₂SO₄ and filtered. After filtration, the filtrate was concentrated under reduced pressure to give a residue. The residue was purified by Prep-HPLC (Condition 11, Gradient 10) to afford N-cyclobutyl-1-[8-(6-methoxypyridazin-4-yl)-6H-isochromeno[3,4-b]pyridin-3-yl]-N-methylpyrrolidin-3-amine (38.8 mg, 37%) as a solid. LCMS (ES, m/z): 444 [M+H]⁺.

### Synthesis of Compound 306

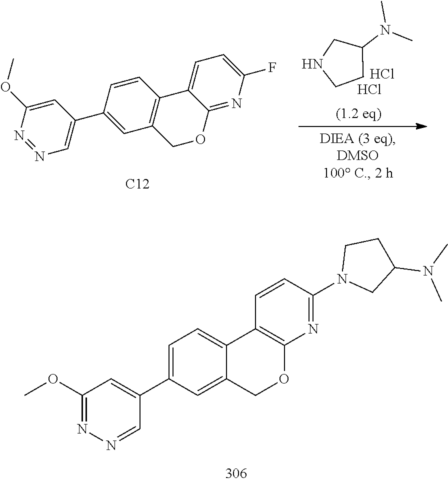

A mixture of 5-{3-fluoro-6H-isochromeno[3,4-b]pyridin-8-yl}-3-methoxypyridazine (50 mg, 0.162 mmol, 1 equiv), N,N-dimethylpyrrolidin-3-amine (22 mg, 0.194 mmol, 1.2 equiv), and DIEA (63 mg, 0.486 mmol, 3 equiv) in DMSO (0.5 mL, 7.040 mmol) was stirred for 2 h at 100° C., then cooled to room temperature. The resulting mixture was poured into water and extracted with ethyl acetate (1×20 mL). The organic layer was dried over anhydrous Na₂SO₄ and filtered. After filtration, the filtrate was concentrated under reduced pressure to give a residue. The residue was purified by Prep-HPLC (Condition 11, Gradient 7) to afford 1-[8-(6-methoxypyridazin-4-yl)-6H-isochromeno[3,4-b]pyridin-3-yl]-N,N-dimethylpyrrolidin-3-amine (8.5 mg 13%) as a solid.

| Compound Structure | Analysis data |
|---|---|
| 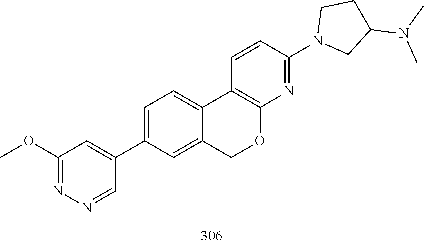 | LCMS (ES, m/z): 404 [M + H]⁺. ¹H NMR (400 MHz, DMSO-d₆) δ 9.33 (d, J = 1.9 Hz, 1H), 8.08 (d, J = 8.6 Hz, 1H), 7.89 (dd, J = 8.2, 2.0 Hz, 1H), 7.84 − 7.75 (m, 2H), 7.50 (d, J = 2.0 Hz, 1H), 6.26 (d, J = 8.5 Hz, 1H), 5.27 (s, 2H), 4.08 (s, 3H), 3.73 − 3.64 (m, 1H), 3.60 (t, J = 9.7 Hz, 1H), 3.30 (s, 1H), 3.13 (dd, J = 10.4, 8.2 Hz, 1H), 2.75 (q, J = 7.7 Hz, 1H), 2.20 (s, 7H), 1.87 − 1.72 (m, 1H). |
| 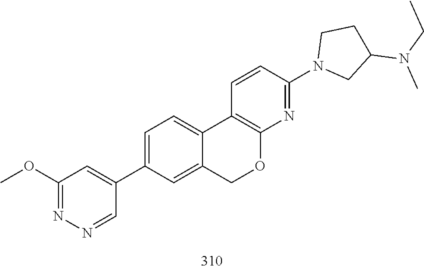 [SMILES: CCN(C)C1CCN(c2ccc3c(n2)OCc2cc(-c4cnnc(OC)c4)ccc2-3)C1] | LCMS (ES, m/z): 418 [M + H]⁺. ¹H NMR (400 MHz, DMSO-d₆) δ 9.33 (d, J = 2.0 Hz, 1H), 8.09 (d, J = 8.4 Hz, 1H), 7.90 (dd, J = 8.1, 2.0 Hz, 1H), 7.84 − 7.75 (m, 2H), 7.50 (d, J = 2.0 Hz, 1H), 6.27 (d, J = 8.5 Hz, 1H), 5.27 (s, 2H), 4.08 (s, 3H), 3.70 (s, 1H), 3.60 (t, J = 9.7 Hz, 1H), 3.35 (s, 1H), 3.29 (s, 2H),3.13 (s, 1H), 3.02 (s, 1H), 2.19 (s, 4H), 1.80 (s, 1H), 1.01 (s, 3H). |
| 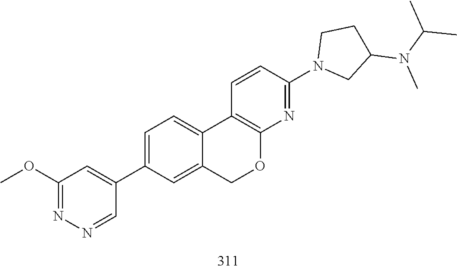 [SMILES: COc1cc(-c2ccc3c(c2)COc2nc(N4CCC(N(C)C(C)C)C4)ccc2-3)cnn1] | LCMS (ES, m/z): 432 [M + H]⁺. ¹H NMR (400 MHz, DMSO-d₆) δ 9.33 (d, J = 1.9 Hz, 1H), 8.08 (d, J = 8.6 Hz, 1H), 7.89 (dd, J = 8.2, 2.0 Hz, 1H), 7.84 − 7.75 (m, 2H), 7.50 (d, J = 2.0 Hz, 1H), 6.26 (d, J = 8.5 Hz, 1H), 5.27 (s, 2H), 4.08 (s, 3H), 3.71 (s, 1H), 3.60 (t, J = 9.8 Hz, 1H), 3.28 (s, 1H), 3.17 (s, 1H), 3.12 − 2.95 (m, 2H), 2.19 − 2.13 (m, 1H), 2.12 (s, 3H), 1.77 (p, J = 10.3 Hz, 1H), 0.98 (dd, J = 6.5, 1.3 Hz, 6H). |

### Example 112: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation OF A new junction with an alternative exon (AJ). Real-time qPCR assays were used to detect these splicing switches and interrogate the potency of various compounds towards different target Genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (i)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_ml)—used for K562/suspension cell lines
GUSB: VIC-PL, ThermoFisher 4326320E (Assay: Hs99999908_ml)—used for K562/suspension cell lines
PPIA: VIC-PL, ThermoFisher 4326316E (Assay: Hs99999904_ml)—used for A673/adherent cell lines

### Probe/Primer Sequences

### Canonical Junction (CJ)

HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/

### Alternative Junction (AJ)

HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABkFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABkFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO2 for 24 hours.

The cells were then gently washed with 50 uL-100 uL Cold Pbs before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. an appropriate volume of 3× lysis buffer (10 mM tRis, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-QpcR reactions, cell lysates were transferred to 384-well qPCR plates containing the master mix according to the table below. The plates were sealed, gently vortexed, and spun down before the run. The volumes were adjusted accordingly in some instances where the reaction was carried in 20 UL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 6, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 6
Modulation of RNA Splicing by Exemplary Compounds

| | HTT AJ | HTT CJ | MYB AJ | MYB |
|---|---|---|---|---|
| Compound | AC₅₀ | IC₅₀ | AC₅₀ | CJ IC₅₀ |
| No. | (nM) | (nM) | (nM) | (nM) |
| 101 | D | — | C | D |
| 102 | D | D | C | D |
| 104 | D | D | D | D |
| 105 | D | D | D | D |
| 108 | A | A | C | B |
| 109 | B | B | — | — |
| 113 | D | D | D | D |
| 114 | A | B | — | — |
| 116 | C | B | C | C |
| 117 | D | D | D | C |
| 118 | D | D | D | D |
| 119 | D | D | — | — |
| 121 | B | B | C | C |
| 124 | C | C | D | C |
| 125 | D | D | D | D |
| 127 | D | D | D | D |
| 128 | D | D | — | — |
| 129 | D | D | D | D |
| 132 | D | D | — | — |
| 134 | C | C | D | C |
| 135 | C | C | D | C |
| 136 | D | D | D | D |
| 137 | C | C | D | D |
| 138 | C | C | — | — |
| 139 | D | D | D | D |
| 140 | D | D | D | D |
| 141 | D | D | — | — |
| 142 | B | B | C | B |
| 143 | B | B | C | B |
| 144 | B | B | B | B |
| 145 | D | D | D | C |
| 146 | C | C | D | D |
| 147 | B | B | C | C |
| 148 | C | C | C | C |
| 149 | A | B | B | B |
| 150 | A | A | B | B |
| 151 | A | A | B | B |
| 152 | A | B | B | B |
| 153 | A | A | A | B |
| 154 | B | B | A | B |
| 155 | A | A | B | B |
| 156 | A | A | B | B |
| 157 | C | C | C | C |
| 158 | B | B | C | B |
| 159 | A | A | A | A |
| 160 | A | A | A | B |
| 161 | D | D | D | D |
| 162 | A | A | A | B |
| 163 | A | A | A | B |
| 164 | A | A | B | B |
| 165 | B | B | B | C |
| 166 | D | D | D | D |
| 167 | D | D | D | D |
| 168 | D | D | D | D |
| 169 | B | B | B | B |
| 170 | C | C | C | D |
| 171 | C | C | C | C |
| 172 | B | B | B | B |
| 173 | D | D | D | D |
| 174 | D | D | D | D |
| 175 | C | D | D | D |
| 176 | C | C | D | D |
| 177 | D | D | D | D |
| 178 | A | A | A | B |
| 179 | B | B | B | B |
| 180 | A | A | B | B |
| 181 | D | D | D | D |
| 182 | D | D | D | D |
| 183 | D | D | D | C |
| 184 | C | C | C | C |
| 185 | D | D | C | D |
| 186 | C | C | D | C |
| 187 | D | C | D | D |
| 188 | B | B | C | C |
| 189 | C | D | D | C |
| 190 | B | A | B | B |
| 191 | C | B | D | C |
| 192 | D | D | D | D |
| 193 | D | D | D | C |
| 194 | C | C | | |
| 195 | — | — | D | C |
| 196 | D | D | D | D |
| 197 | D | C | C | D |
| 198 | B | B | B | B |
| 199 | C | C | D | D |
| 200 | A | A | B | B |
| 201 | B | B | C | B |
| 202 | D | C | D | C |
| 203 | C | C | — | — |
| 204 | D | C | D | C |
| 205 | B | C | — | — |
| 206 | B | B | C | B |
| 207 | — | — | B | C |
| 208 | — | — | B | B |
| 209 | — | — | C | B |
| 210 | — | D | — | D |
| 211 | —— | A | — | B |
| 212 | — | A | — | B |
| 213 | — | B | —— | C |
| 214 | — | D | — | D |
| 217 | C | B | D | C |
| 218 | A | A | B | B |
| 219 | B | B | D | B |
| 220 | B | B | B | B |
| 221 | D | D | D | C |
| 222 | D | D | D | D |
| 223 | A | A | A | B |
| 224 | C | C | D | D |
| 225 | C | B | D | D |
| 226 | A | A | D | B |
| 227 | D | D | D | D |
| 228 | C | B | C | C |
| 229 | D | D | D | D |
| 230 | B | B | C | B |
| 231 | B | B | C | B |
| 232 | A | A | A | B |
| 233 | B | A | C | B |
| 234 | A | A | B | B |
| 235 | D | D | D | D |
| 236 | A | A | A | B |
| 238 | C | B | C | C |
| 239 | C | B | C | C |
| 240 | B | A | C | C |
| 241 | C | C | C | C |
| 242 | A | A | A | C |
| 243 | A | A | B | B |
| 244 | A | B | B | B |
| 245 | A | A | A | A |
| 246 | D | D | C | D |
| 247 | A | A | D | B |
| 248 | B | B | C | C |
| 249 | C | C | D | D |
| 250 | B | C | C | C |
| 251 | A | A | B | B |
| 252 | C | B | C | C |
| 253 | A | B | B | B |
| 254 | A | A | B | B |
| 255 | C | B | C | C |
| 256 | B | B | C | C |
| 257 | A | A | B | B |
| 258 | B | B | B | C |
| 260 | A | A | A | B |
| 261 | D | D | D | D |
| 263 | D | D | D | D |
| 264 | B | B | C | C |
| 265 | A | A | A | B |
| 266 | D | D | D | D |
| 267 | C | B | D | D |
| 268 | C | C | D | D |
| 269 | B | A | C | B |
| 270 | A | A | B | B |
| 271 | A | A | B | B |
| 272 | C | C | D | D |
| 273 | D | D | D | D |
| 274 | D | C | D | D |
| 275 | B | B | B | B |
| 276 | B | B | C | C |
| 277 | B | B | C | C |
| 278 | B | A | B | B |
| 279 | B | B | C | C |
| 280 | B | B | B | C |
| 281 | A | A | B | B |
| 282 | B | B | B | B |
| 283 | C | B | C | C |
| 284 | D | D | D | D |
| 285 | B | A | B | B |
| 286 | D | D | D | D |
| 287 | B | B | C | C |
| 288 | C | C | D | D |
| 289 | C | B | C | B |
| 290 | B | A | B | B |
| 292 | D | D | D | D |
| 293 | D | D | D | D |
| 294 | B | A | C | B |
| 295 | A | A | C | B |
| 296 | B | B | B | B |
| 297 | B | A | B | B |
| 298 | B | B | B | C |
| 299 | B | B | C | C |
| 300 | C | C | D | D |
| 301 | B | C | C | C |
| 302 | D | D | D | D |
| 303 | B | A | B | B |
| 304 | A | A | B | B |
| 305 | B | B | C | C |
| 306 | C | B | C | C |
| 307 | B | B | B | C |
| 308 | C | B | C | C |
| 309 | A | A | B | B |
| 310 | B | B | C | B |
| 311 | B | B | B | C |
| 312 | C | B | C | C |
| 315 | D | D | D | D |
| 316 | B | A | B | B |
| 317 | B | B | B | B |
| 318 | B | A | B | B |
| 320 | C | C | C | D |
| 321 | B | B | C | C |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, MYB, and Target C) analyzed in this panel are reported as IC₅₀ (compound concentration having 50% response in CJ decrease).

A summary of the results from the panel is illustrated in Table 7, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 7
Modulation of RNA Splicing by Exemplary Compounds

| Compound | | | Target | |
|---|---|---|---|---|
| No. | HTT | SMN2 | C | MYB |
| 101 | — | — | D | D |
| 102 | D | — | D | D |
| 104 | D | — | D | D |
| 105 | D | D | — | D |
| 108 | A | A | A | B |
| 112 | — | — | D | — |
| 113 | D | D | D | D |
| 114 | B | — | A | — |
| 116 | B | B | B | C |
| 117 | D | D | — | C |
| 118 | D | D | — | D |
| 119 | D | — | C | — |
| 121 | B | B | B | C |
| 124 | C | C | C | C |
| 125 | D | D | D | D |
| 127 | D | D | — | D |
| 128 | D | — | D | — |
| 129 | D | D | D | D |
| 132 | D | — | D | — |
| 134 | C | C | A | C |
| 135 | C | C | C | C |
| 136 | D | D | D | D |
| 137 | D | D | C | D |
| 138 | C | — | C | — |
| 139 | D | D | D | D |
| 140 | D | D | — | D |
| 141 | D | — | C | — |
| 142 | B | B | C | B |
| 143 | B | B | B | B |
| 144 | B | D | B | B |
| 145 | D | C | C | C |
| 146 | C | D | D | D |
| 147 | B | D | B | C |
| 148 | C | B | C | C |
| 149 | B | B | B | B |
| 150 | A | D | B | B |
| 151 | A | A | B | B |
| 152 | B | A | B | B |
| 153 | A | A | A | B |
| 154 | B | B | B | B |
| 155 | A | A | B | B |
| 156 | A | B | B | B |
| 157 | C | B | C | C |
| 158 | B | B | B | B |
| 159 | A | A | A | A |
| 160 | A | A | A | B |
| 161 | D | D | D | D |
| 162 | A | A | A | B |
| 163 | A | A | B | B |
| 164 | A | A | B | B |
| 165 | B | A | B | C |
| 166 | D | C | D | D |
| 167 | D | D | D | D |
| 168 | D | D | D | D |
| 169 | B | B | B | B |
| 170 | C | C | D | D |
| 171 | C | B | C | C |
| 172 | B | A | B | B |
| 173 | D | C | D | D |
| 174 | D | C | D | D |
| 175 | D | C | D | D |
| 176 | C | C | D | D |
| 177 | D | D | D | D |
| 178 | A | A | B | B |
| 179 | B | B | B | B |
| 180 | A | A | B | B |
| 181 | D | D | D | D |
| 182 | D | D | D | D |
| 183 | D | B | D | C |
| 184 | C | C | C | C |
| 185 | D | C | D | D |
| 186 | C | B | D | C |
| 187 | C | C | D | D |
| 188 | B | B | C | C |
| 189 | D | C | D | C |
| 190 | A | A | B | B |
| 191 | B | B | C | C |
| 192 | D | D | D | D |
| 193 | C | D | D | C |
| 194 | D | D | D | — |
| 195 | B | B | C | C |
| 196 | D | D | D | D |
| 197 | C | D | D | D |
| 198 | B | B | C | B |
| 199 | C | C | D | D |
| 200 | A | A | B | B |
| 201 | B | A | B | B |
| 202 | C | D | D | C |
| 204 | C | C | D | C |
| 206 | B | B | C | B |
| 207 | D | D | D | C |
| 208 | A | A | B | B |
| 209 | B | B | C | B |
| 217 | B | B | C | C |
| 218 | A | A | B | B |
| 219 | B | B | B | B |
| 220 | B | B | C | B |
| 221 | D | D | D | C |
| 222 | D | D | D | D |
| 223 | A | A | A | B |
| 224 | C | C | D | D |
| 225 | B | C | D | D |
| 226 | A | A | B | B |
| 227 | D | D | D | D |
| 228 | B | B | C | C |
| 229 | D | D | D | D |
| 230 | B | B | C | B |
| 231 | B | A | B | B |
| 232 | A | A | A | B |
| 233 | A | A | B | B |
| 234 | A | A | B | B |
| 235 | D | D | C | D |
| 236 | A | A | B | B |
| 238 | B | C | C | C |
| 239 | B | B | C | C |
| 240 | A | B | B | C |
| 241 | C | C | C | C |
| 242 | A | A | A | B |
| 243 | A | A | B | B |
| 244 | B | B | B | B |
| 245 | A | A | A | A |
| 246 | D | C | D | D |
| 247 | A | A | B | B |
| 248 | B | B | C | C |
| 249 | C | B | D | D |
| 250 | C | B | C | C |
| 251 | A | A | A | B |
| 252 | B | B | C | C |
| 253 | B | A | B | B |
| 254 | A | A | B | B |
| 255 | B | B | C | C |
| 256 | B | B | B | C |
| 257 | A | A | A | B |
| 258 | B | B | B | C |
| 260 | A | A | B | B |
| 261 | D | D | D | D |
| 263 | D | D | D | D |
| 264 | B | B | B | C |
| 265 | A | A | A | B |
| 266 | D | C | D | D |
| 267 | B | B | C | D |
| 268 | C | C | D | D |
| 269 | A | A | B | B |
| 270 | A | A | B | B |
| 271 | A | A | B | B |
| 272 | C | C | D | D |
| 273 | D | D | D | D |
| 274 | C | B | D | D |
| 275 | B | A | B | B |
| 276 | B | B | B | C |
| 277 | B | B | C | C |
| 278 | A | A | B | B |
| 279 | B | B | C | C |
| 280 | B | B | B | C |
| 281 | A | A | B | B |
| 282 | B | B | B | B |
| 283 | B | B | C | C |
| 284 | D | C | D | D |
| 285 | A | A | B | B |
| 286 | D | D | D | D |
| 287 | B | B | C | C |
| 288 | C | C | D | D |
| 289 | B | A | C | B |
| 290 | A | A | B | B |
| 292 | D | C | C | D |
| 293 | D | D | D | D |
| 294 | A | B | B | B |
| 295 | A | A | B | B |
| 296 | B | A | B | B |
| 297 | A | A | B | B |
| 298 | B | A | C | C |
| 299 | B | B | C | C |
| 300 | C | B | C | D |
| 301 | C | C | C | C |
| 302 | D | D | D | D |
| 303 | A | A | B | B |
| 304 | A | A | B | B |
| 305 | B | B | C | C |
| 306 | B | B | C | C |
| 307 | B | A | B | C |
| 308 | B | B | C | C |
| 309 | A | A | B | B |
| 310 | B | B | C | B |
| 311 | B | B | C | C |
| 312 | B | B | D | C |
| 315 | D | D | D | D |
| 316 | A | A | B | B |
| 317 | B | A | B | B |
| 318 | A | A | B | B |
| 320 | C | B | C | D |
| 321 | B | B | C | C |

### Example 113: Evaluating effect of exemplary compounds on protein abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)
Synthego Engineered Cells Knock-In Clones

TABLE 8
Design of genetically modified HiBiT cell lines

| | | | Guide | Guide | |
|---|---|---|---|---|---|
| Cell | | Modifi- | RNA | RNA cut | Donor |
| Line | Gene | cation | Sequence | location | Sequence |
| K562 | MYB | HiBiT | GCGCCA | chr6: | CGGTGCGGTCC |
| | | | TGGCCC | 135,181,526 | CCGCGGCTCTC |
| | | | GAAGAC | | GGCGGAGCCCC |
| | | | CC | | GCGCCCGCCGC |
| | | | | | GCCATGgtgag |
| | | | | | cggctggcggc |
| | | | | | tgttcaagaag |
| | | | | | attagcGGCAG |
| | | | | | CTCCGGAGGAT |
| | | | | | CTAGCGGCGCC |
| | | | | | CGAAGACCCCG |
| | | | | | GCACAGgtaac |
| | | | | | ggggagccggg |
| | | | | | cgggcggccga |
| | | | | | ggg |
| K562 | HTT | HiBiT | CAGCTT | chr4: | CGAGTCGGCCC |
| | | | TTCCAG | 3,074,830 | GAGGCCTCCGG |
| | | | GGTCGC | | GGACTGCCGTG |
| | | | CA | | CCGGGCGGGAG |
| | | | | | ACCGCCATGgt |
| | | | | | gagcggctggc |
| | | | | | ggctgttcaag |
| | | | | | aagattagcGG |
| | | | | | CAGCTCCGGAG |
| | | | | | GATCTAGCGGC |
| | | | | | GCGACCCTGGA |
| | | | | | AAAGCTGATGA |
| | | | | | AGGCCTTCGAG |
| | | | | | TCCCTCAAGTC |
| | | | | | CTTCCA |

### Description:

Cells were maintained in IMDM with 10% FBS. Before the assay, cells were diluted with phenolphthalein-free growth media (IMDM+100 FBS media) and were seeded in a 384-well plate at a density of 10000 cells/well (for each cell line listed in Table 8). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25% o. Treated cell plates were placed in an incubator at 37° C. with 500 CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 9, the percent response for each respective cell line was calculated at each compound concentration as follows:

$$\% response = 100 * ( S - PC ) / ( NC - PC )$$

For the normalized response at each concentration, a four-parameter logistical regression was fit to the data and the response was interpolated at the 5000 value to determine a concentration for protein abundance at 50% o (IC₅₀) the untreated control.

A summary of the results for protein abundance is illustrated in Table 9, wherein A represents <100 nM; B represents 100-1000 nM; C represents 1000-9999 nM; and D represents greater than 10 μM.

TABLE 9

| Compound | | | Target |
|---|---|---|---|
| No. | HTT | MYB | C |
| 101 | C | C | C |
| 102 | C | C | C |
| 104 | C | C | C |
| 105 | C | C | C |
| 108 | A | A | A |
| 113 | D | C | D |
| 116 | B | C | C |
| 117 | C | C | C |
| 118 | C | C | C |
| 119 | B | C | C |
| 121 | A | C | B |
| 124 | C | C | C |
| 125 | C | B | C |
| 129 | D | D | D |
| 134 | C | C | C |
| 142 | B | C | C |
| 143 | B | C | B |
| 144 | A | B | A |
| 145 | C | C | C |
| 141 | C | A | C |
| 146 | C | C | C |
| 147 | B | B | B |
| 148 | C | C | C |
| 149 | B | B | B |
| 151 | A | B | A |
| 152 | A | B | B |
| 153 | A | A | A |
| 154 | A | B | B |
| 155 | A | B | A |
| 156 | A | B | B |
| 157 | B | C | C |
| 158 | A | B | C |
| 159 | A | A | A |
| 160 | A | B | A |
| 161 | D | D | D |
| 162 | A | B | A |
| 163 | A | A | A |
| 164 | A | B | A |
| 165 | A | B | B |
| 166 | C | C | D |
| 206 | A | B | B |
| 207 | C | C | C |
| 208 | A | B | A |
| 218 | B | A | B |
| 285 | B | B | B |
| 286 | C | C | C |
| 287 | B | B | B |
| 290 | A | B | A |
| 305 | B | C | B |
| 306 | B | C | B |
| 307 | B | B | B |

### Example 114: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials:

Promega CellTiter-Gbo® 2.0 Cell Viability Assay (cat #G9241)
Corning 384-well TC-treated microplates (cat #3570)

### Description:

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I)) were first serially diluted in DMSO then diluted 1:100 with IMDM+10% FBS. The final concentration of DMSO was 0.1% in each well. The cells were incubated for 72 hours at 37° C. and 5% CO₂ before assaying with Cell Titer Glo 2.0 reagent. Viability may be assessed using an exemplary paradigm, in which A represents <100 nM; B represents 100-1000 nM; C represents 1000-9999 nM; and D represents greater than 10 μM in K562 cells.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 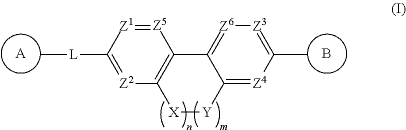
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L is absent, C₁-C₆-alkylene, C₁-C₆-heteroalkylene, —O—, —S—, —C(O)—, —N(R⁴)—, —N(R⁴)C(O)— or —C(O)N(R⁴)—, wherein each alkylene and heteroalkylene is optionally substituted with one or more R⁵;
Z¹, Z², Z³, Z⁴, Z⁵, and Z⁶ are each independently C(R⁶) or N;
X and Y are each independently O, C(R^{7a})(R^{7b}), or N(R^{7c}), wherein one of X and Y is independently C(R^{7a})(R^{7b});
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;
each R⁴ is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R¹²;
each R⁵ is independently C₁-C₆-alkyl, C₁-C₆-heteroalkyl, C₁-C₆-haloalkyl, cycloalkyl, heterocyclyl, halo, cyano, oxo, —OR^{A}, —NR^{B}R^{C}, —C(O)R^{D}, or —C(O)OR^{D};
R⁶ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —OR^{A}, —NR^{B}R^{C}, —C(O)R^{D}, or —C(O)OR^{D};
R^{7a} and R^{7b} are each independently hydrogen, C₁-C₆-alkyl, or cycloalkyl; or
R^{7a} and R^{7b}, together with the carbon atom to which they are attached, form an oxo group;
R^{7c} is hydrogen or C₁-C₆-alkyl;
each R⁸ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₁-C₆ alkylene-heteroaryl, —OR^{A}, —C(O)R^{D}, or —S(O)ₓR^{D};
R^{B} and R^{C} together with the atom to which they are attached form a 3-7-membered heterocyclyl or heteroaryl ring optionally substituted with one or more R¹⁰;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, or C₁-C₆ alkylene-heteroaryl;
each R¹⁰ is C₁-C₆-alkyl, halo, cyano, oxo, or —OR^{A1};
each R¹¹ is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, halo, cyano, oxo, or —OR^{A};
each R¹² is independently deuterium, halo, cyano, —OR^{A}, —NR^{B}R^{C}, —NR^{B}C(O)R^{D}, —C(O)NR^{B}R^{C}, —C(O)R^{D}, —C(O)OR^{D}, or —C(O)R^{D};
each R^{A1} is hydrogen or C₁-C₆-alkyl;
each of m and n is independently 1 or 2; and
x is 0, 1, or 2.

2. The compound of claim 1, wherein each of A and B is independently heteroaryl or heterocyclyl, each of which is optionally substituted with one or more R¹.

3. The compound of any one of the preceding claims, wherein one of A and B is independently a monocyclic heteroaryl or bicyclic heteroaryl, each of which is optionally substituted with one or more R¹.

4. The compound of any one of the preceding claims, wherein one of A and B is independently a monocyclic heteroaryl optionally substituted with one or more R¹.

5. The compound of any one of the preceding claims, wherein one of A and B is independently a nitrogen-containing heteroaryl optionally substituted with one or more R¹.

6. The compound of any one of the preceding claims, wherein one of A and B is independently a 5-6 membered heteroaryl optionally substituted with one or more R¹.

7. The compound of any one of the preceding claims, wherein one of A and B is independently selected from 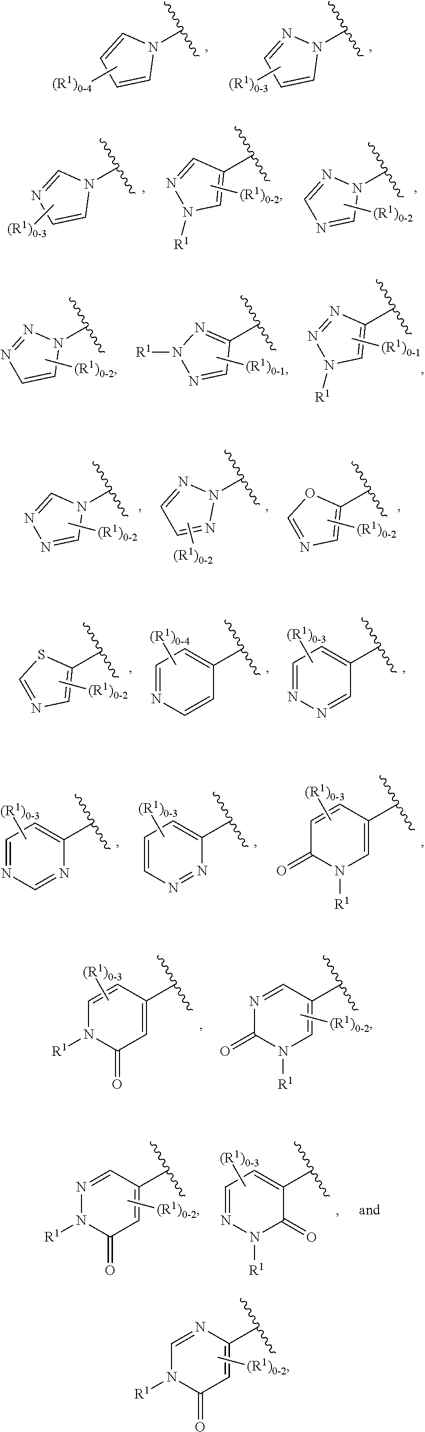
wherein R¹ is as described in claim 1.

8. The compound of any one of the preceding claims, wherein one of A and B is independently selected from 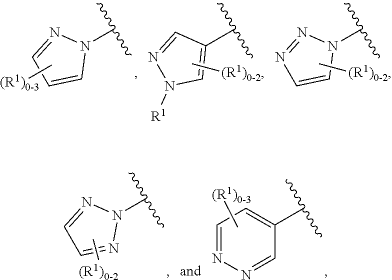
wherein R¹ is as described in claim 1.

9. The compound of any one of the preceding claims, wherein one of A and B is independently selected from 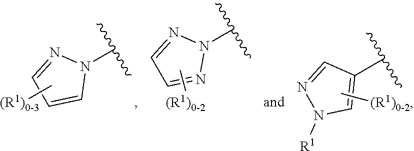
wherein R¹ is as described in claim 1.

10. The compound of any one of the preceding claims, wherein A is selected from 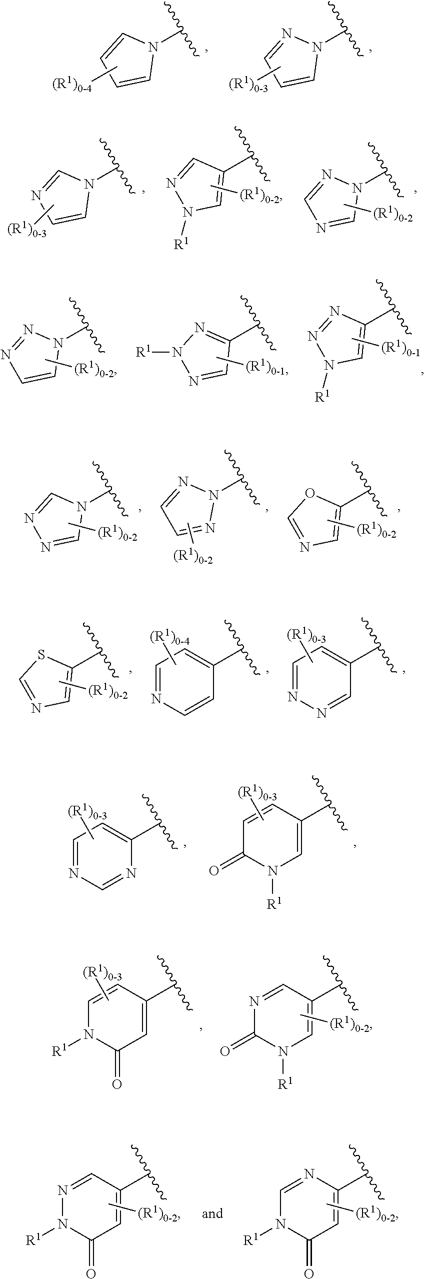
wherein R¹ is as described in claim 1, wherein R¹ is as described in claim 1.

11. The compound of any one of the preceding claims, wherein B is selected from 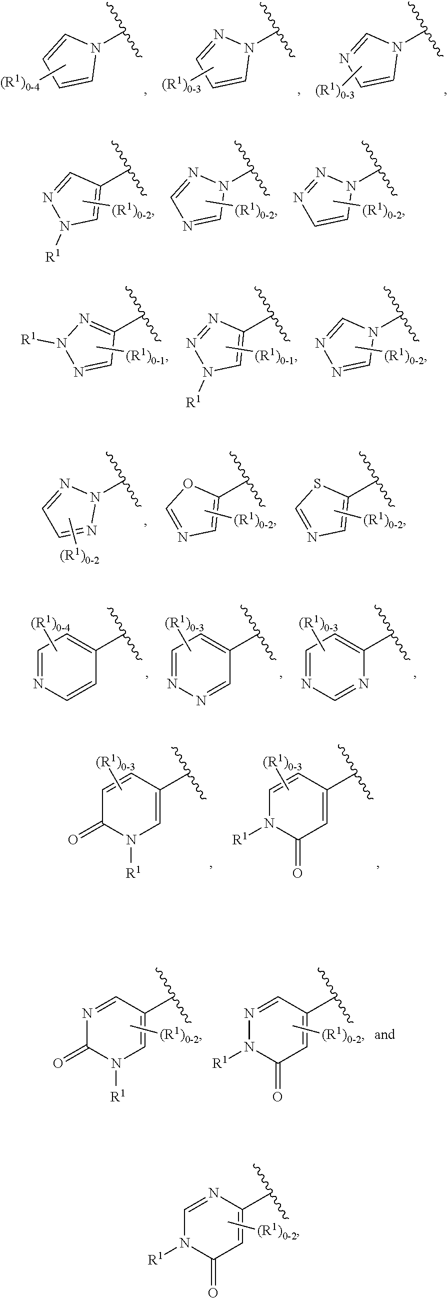
wherein R¹ is as described in claim 1, wherein R¹ is as described in claim 1.

12. The compound of any one of the preceding claims, B is selected from 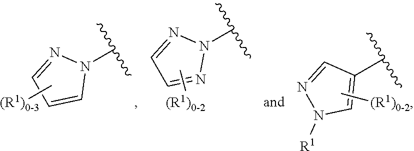
wherein R¹ is as described in claim 1.

13. The compound of any one of the preceding claims, wherein one of A and B is independently selected from

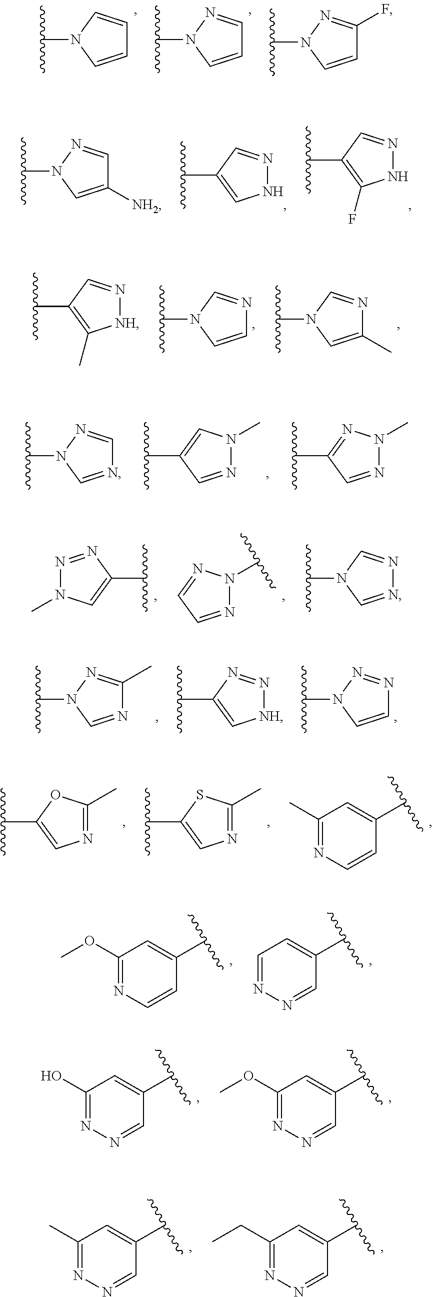

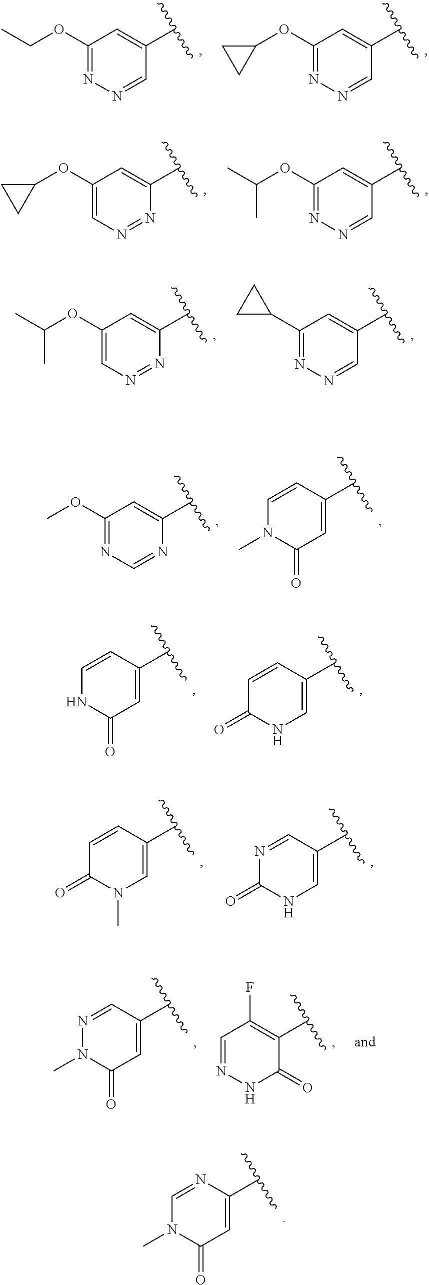

14. The compound of any one of the preceding claims, wherein one of A and B is independently selected from

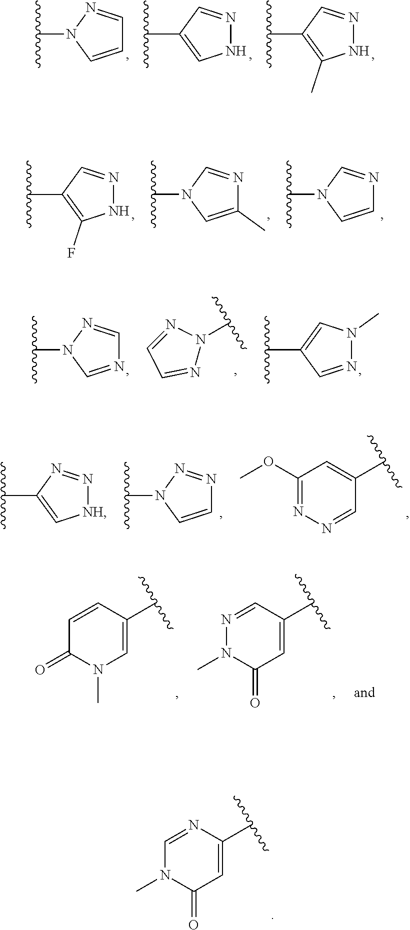

15. The compound of any one of the preceding claims, wherein one of A and B is independently selected from

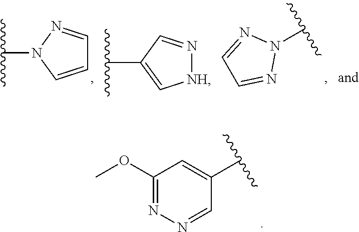

16. The compound of any one of the preceding claims, wherein A is selected from

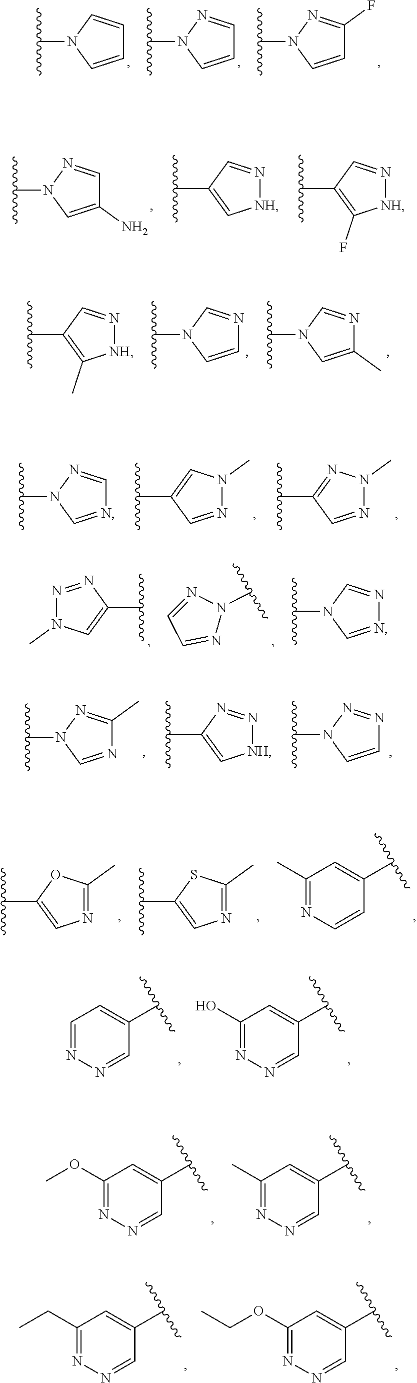

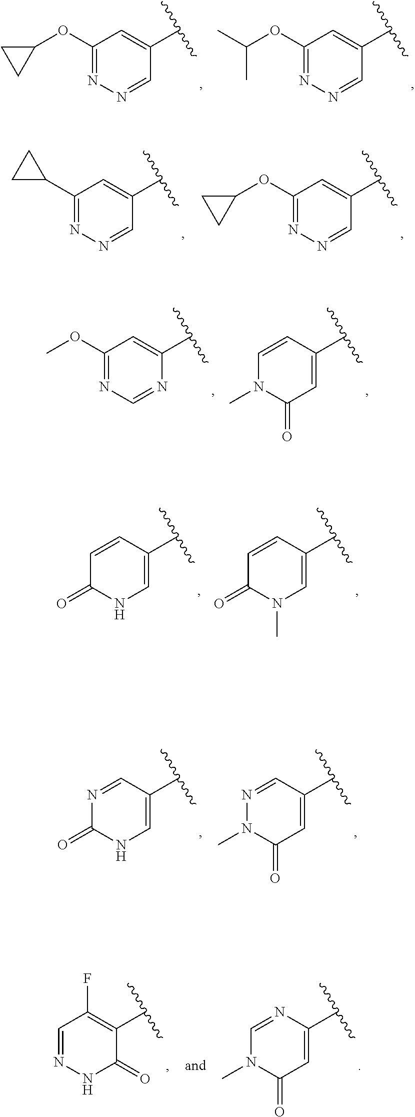

17. The compound of any one of the preceding claims, wherein B is selected from

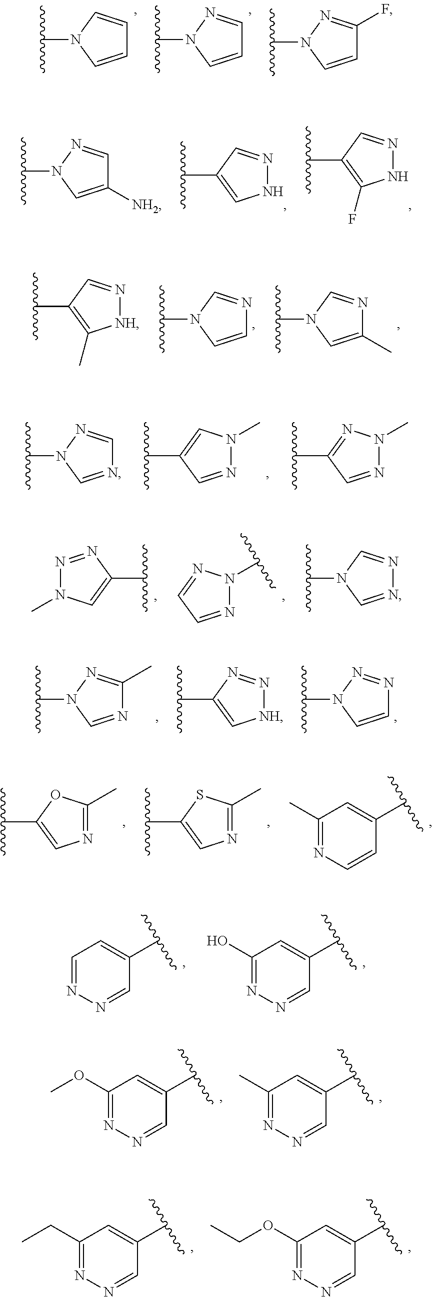

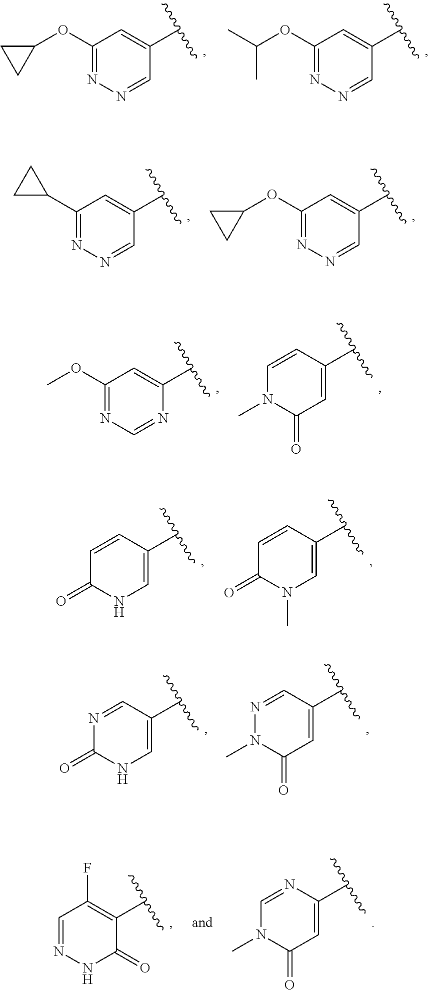

18. The compound of any one of the preceding claims, wherein B is selected from

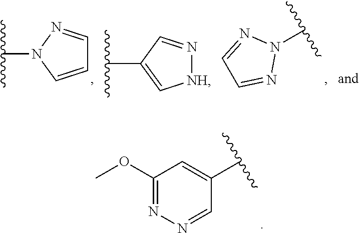

19. The compound of any one of the preceding claims, wherein one of A and B is independently a monocyclic heterocyclyl or bicyclic heterocyclyl, each of which is optionally substituted with one or more R¹.

20. The compound of any one of the preceding claims, wherein one of A and B is independently a nitrogen-containing heterocyclyl optionally substituted with one or more R¹.

21. The compound of any one of the preceding claims, wherein one of A and B is independently a 4-10 membered heterocyclyl optionally substituted with one or more R¹.

22. The compound of any one of the preceding claims, wherein one of A and B is independently selected from 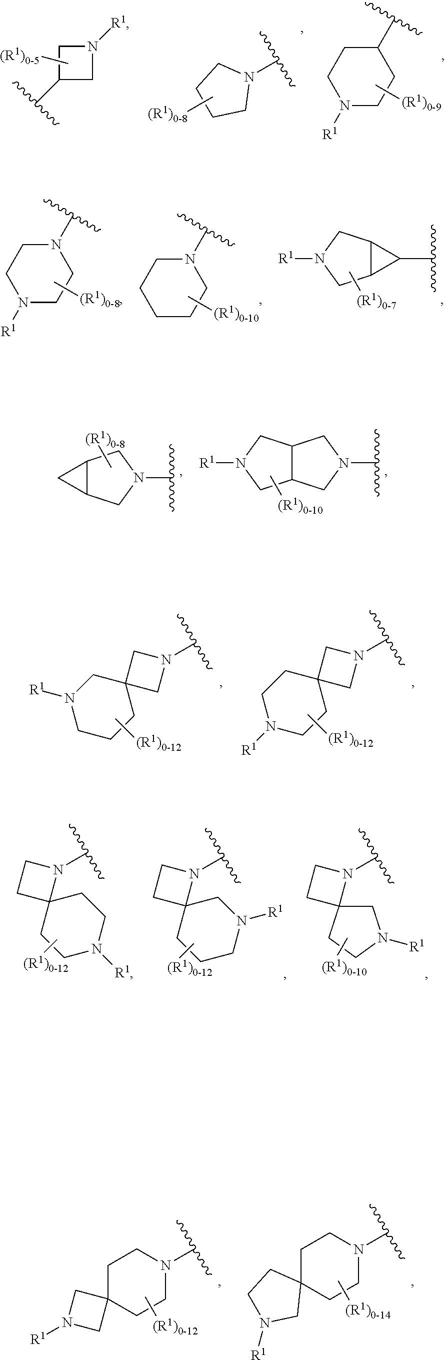 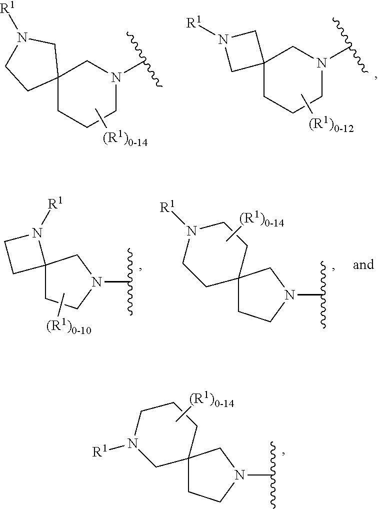
wherein R¹ is as described in claim 1.

23. The compound of any one of the preceding claims, wherein one of A and B is independently selected from 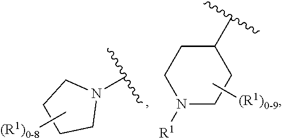
wherein R¹ is as described in claim 1.

24. The compound of any one of the preceding claims, wherein one of A and B is 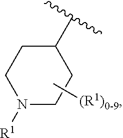
wherein R¹ is as described in claim 1.

25. The compound of any one of the preceding claims, wherein A is selected from 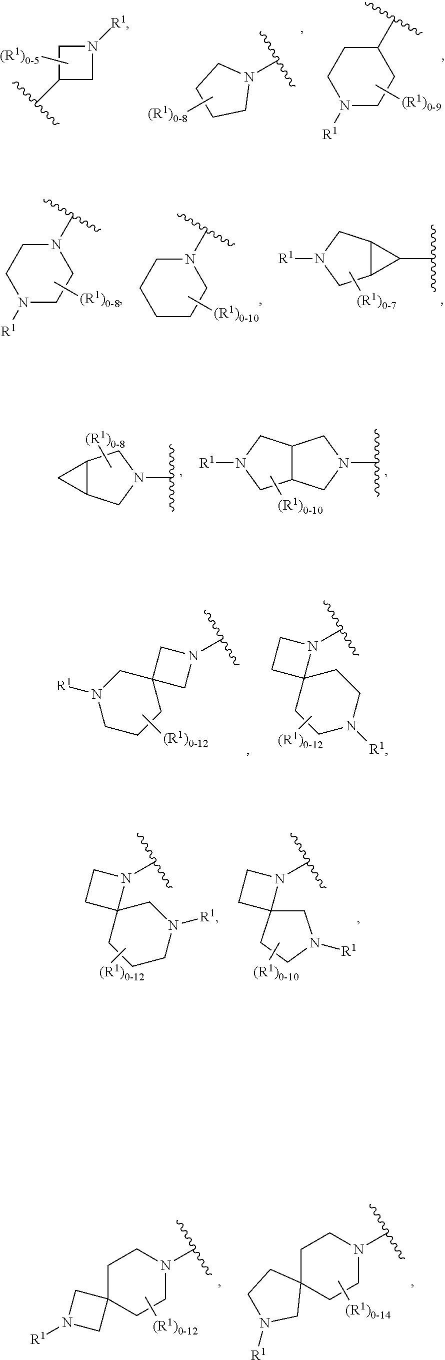 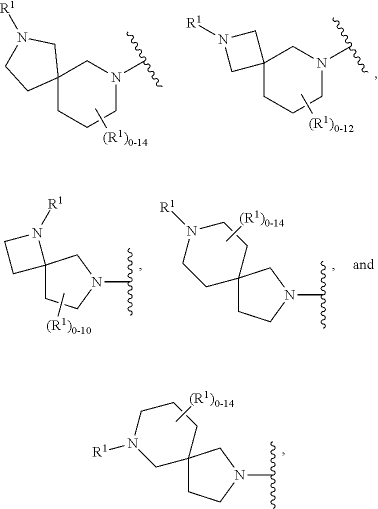
and wherein R¹ is as described in claim 1.

26. The compound of any one of the preceding claims, wherein B is selected from 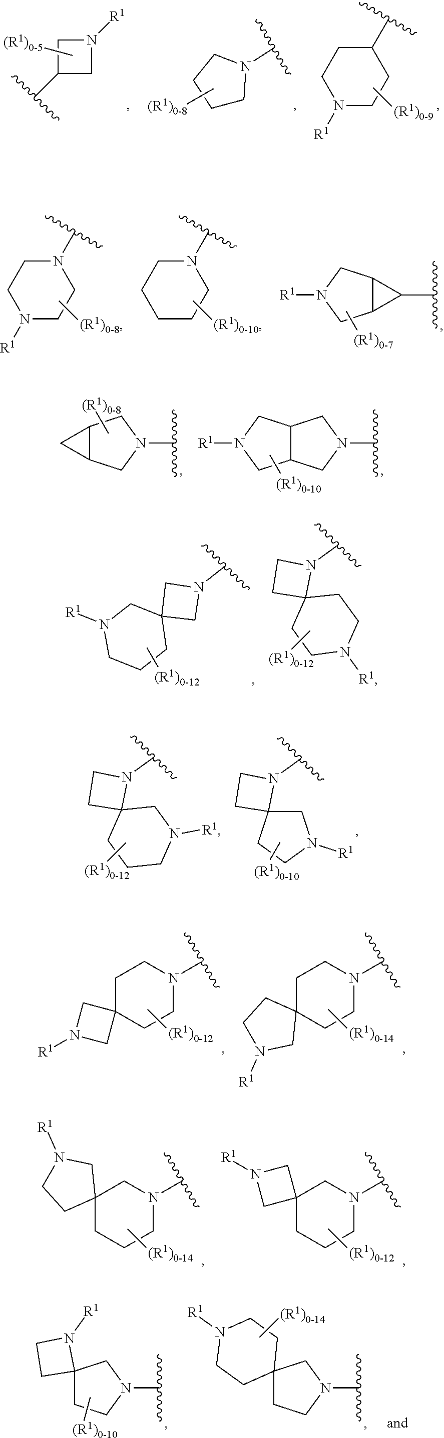 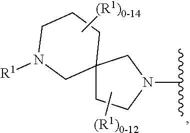
and wherein R¹ is as described in claim 1.

27. The compound of any one of the preceding claims, wherein A is selected from 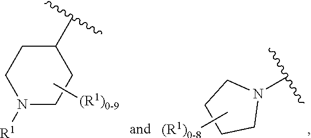
and wherein R¹ is as described in claim 1.

28. The compound of any one of the preceding claims, wherein one of A and B is independently selected from H

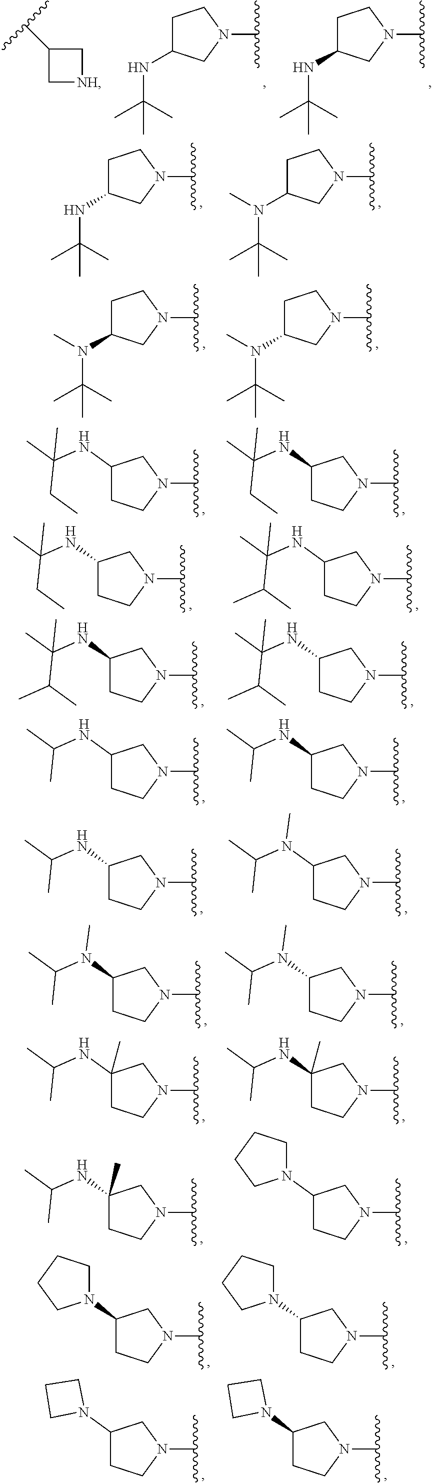

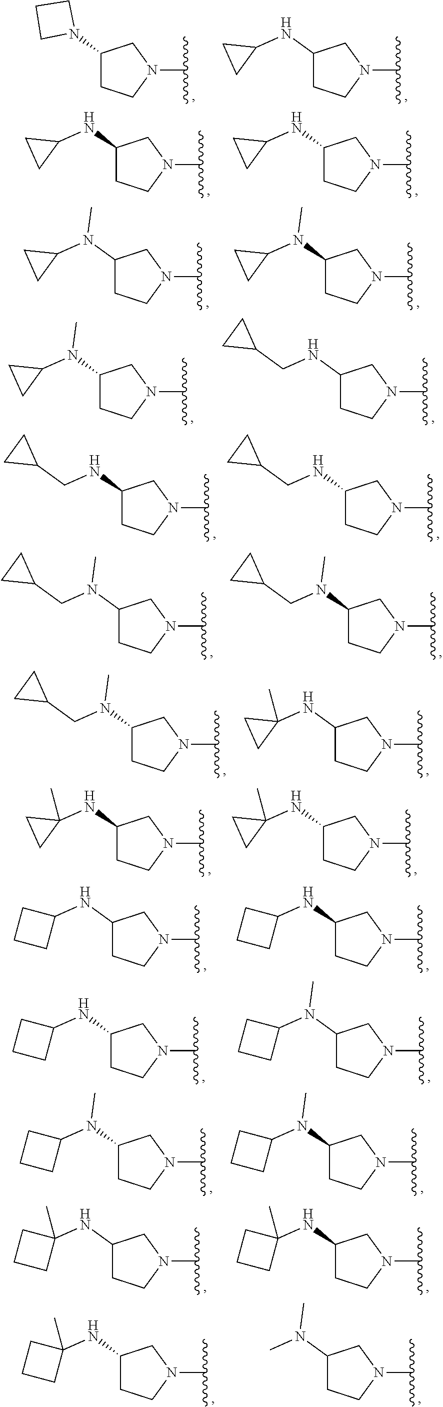

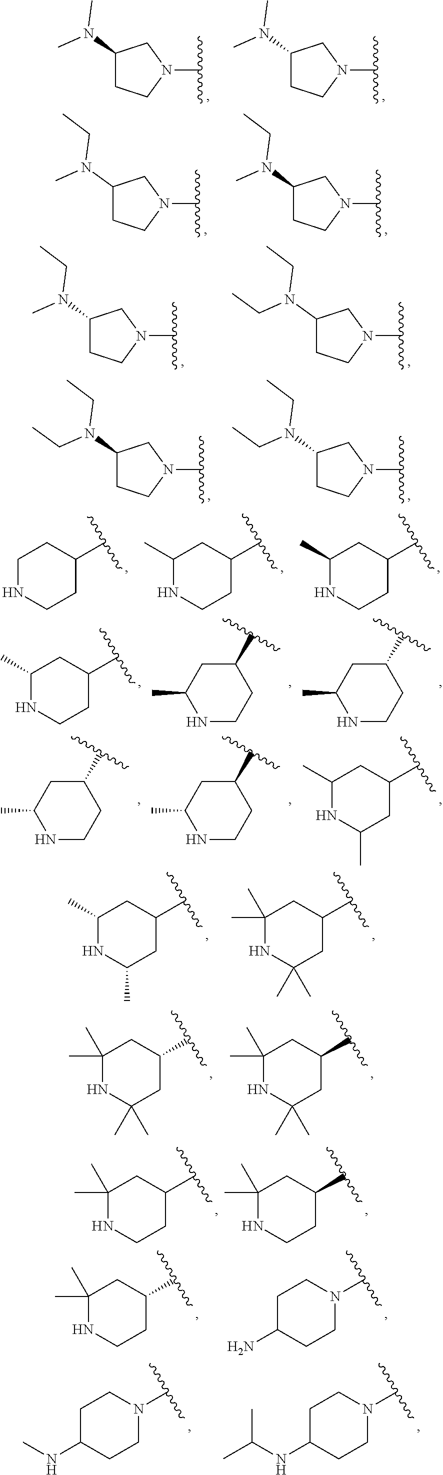

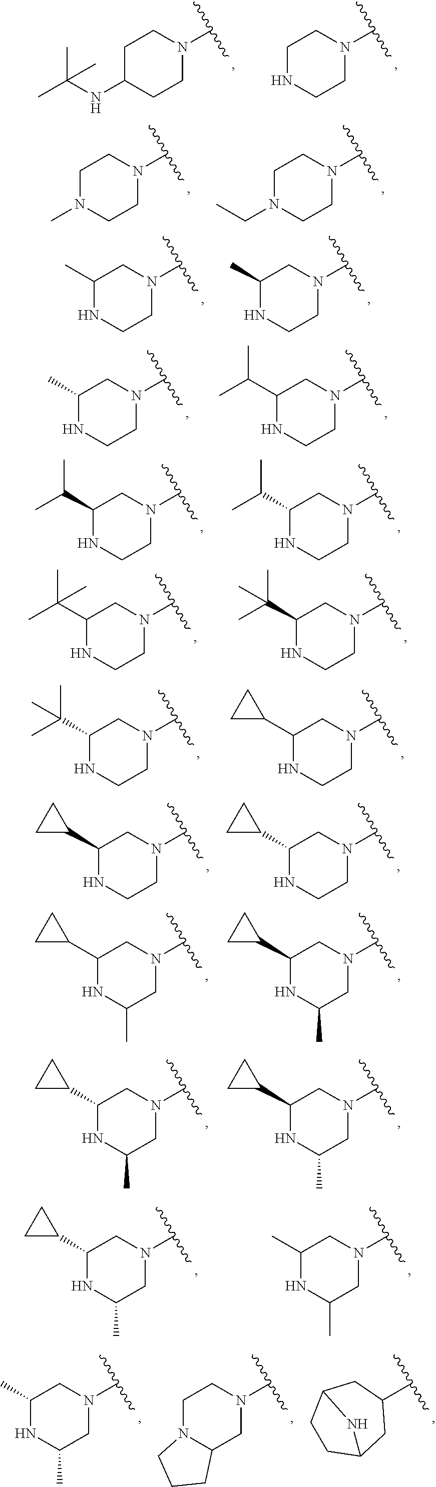

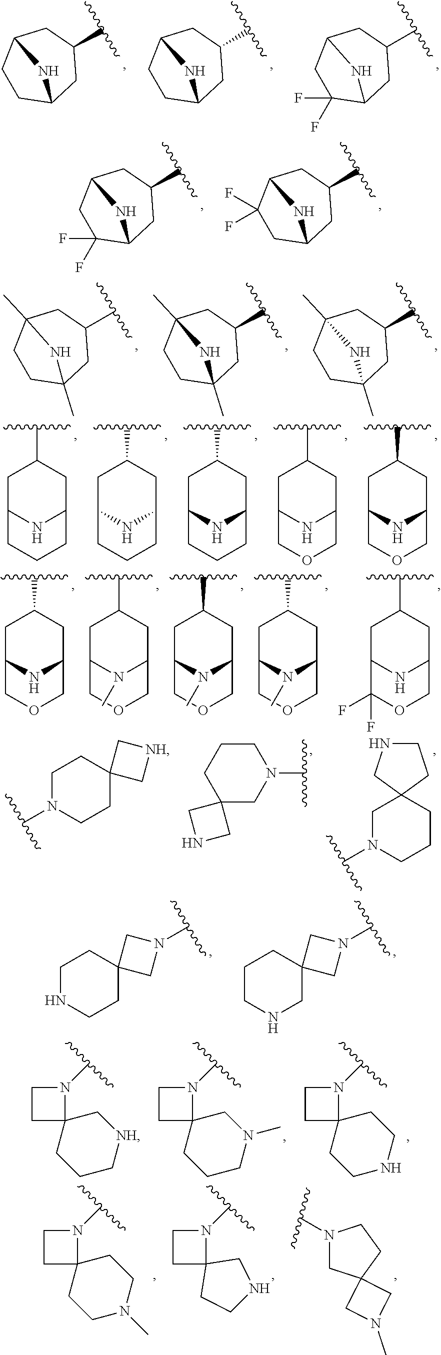

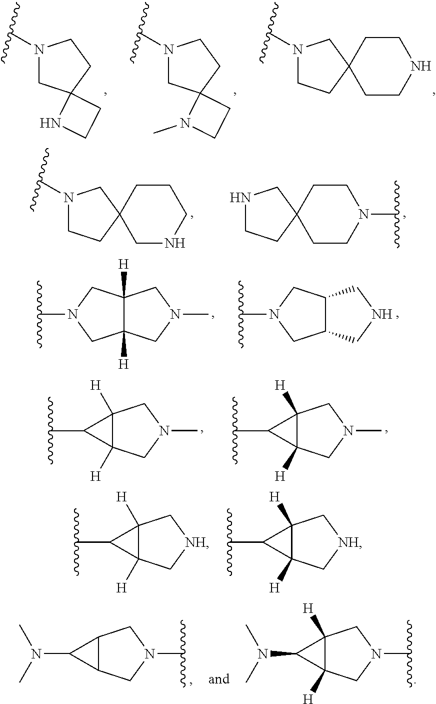

29. The compound of any one of the preceding claims, wherein one of A and B is independently selected from

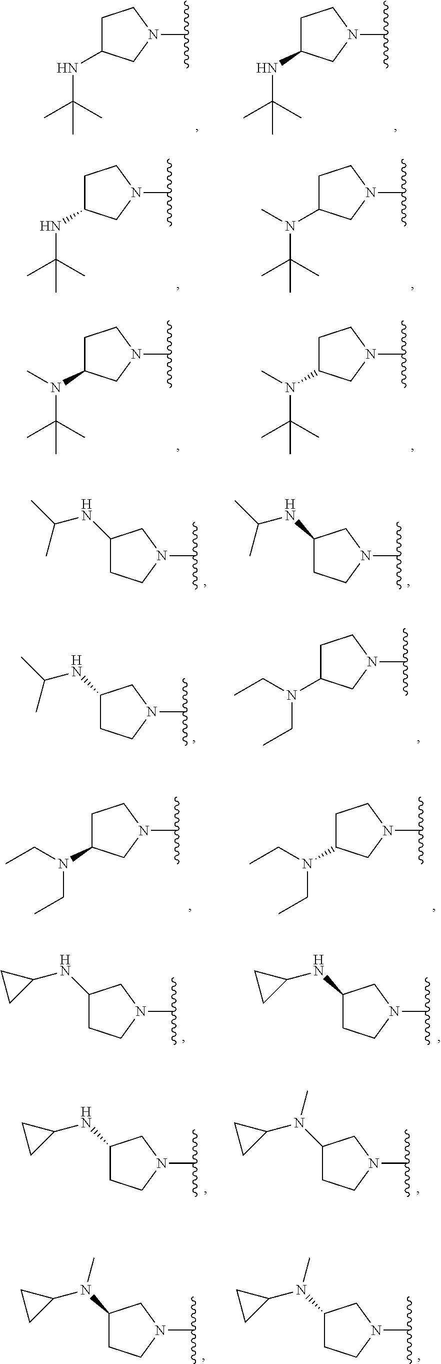

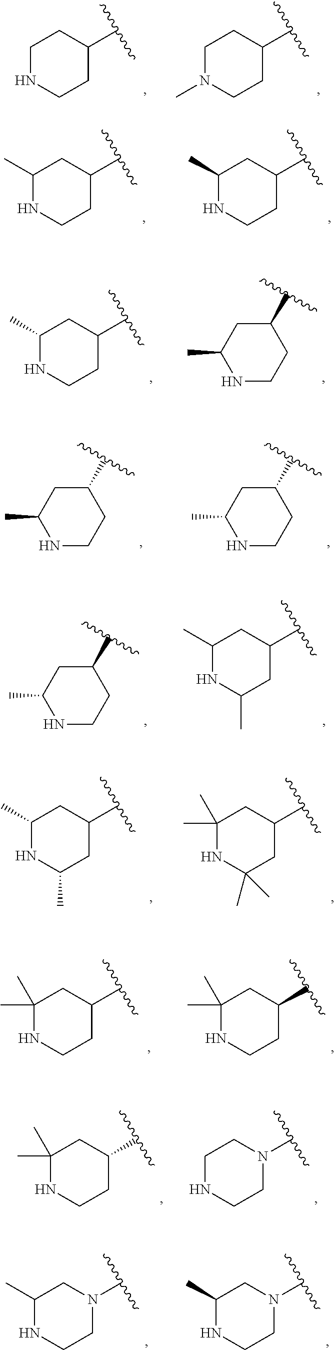

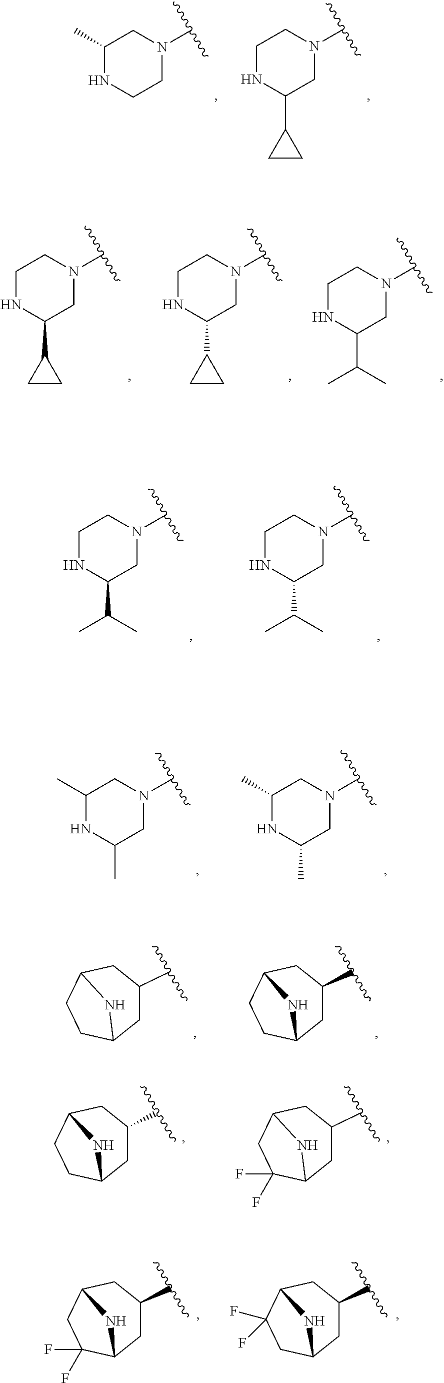

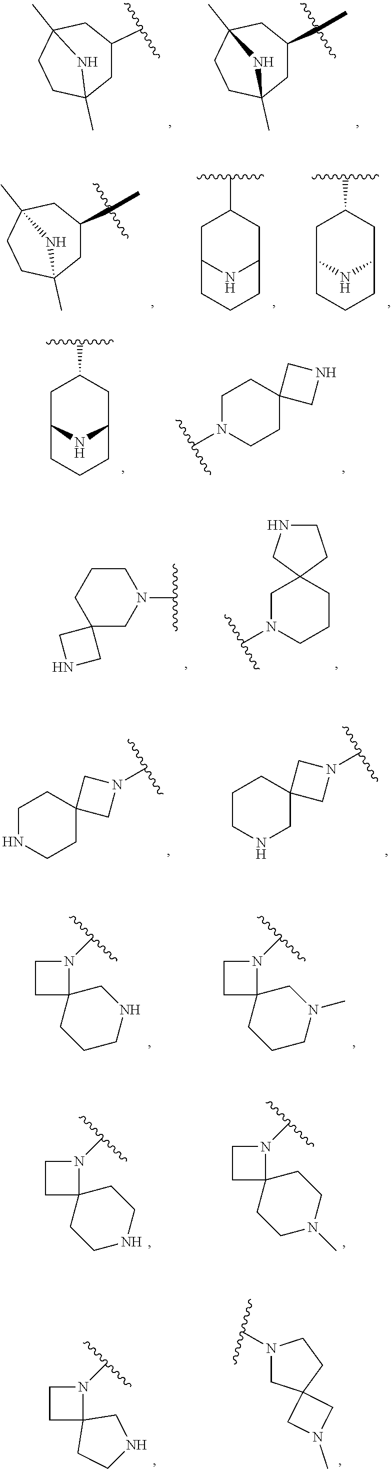

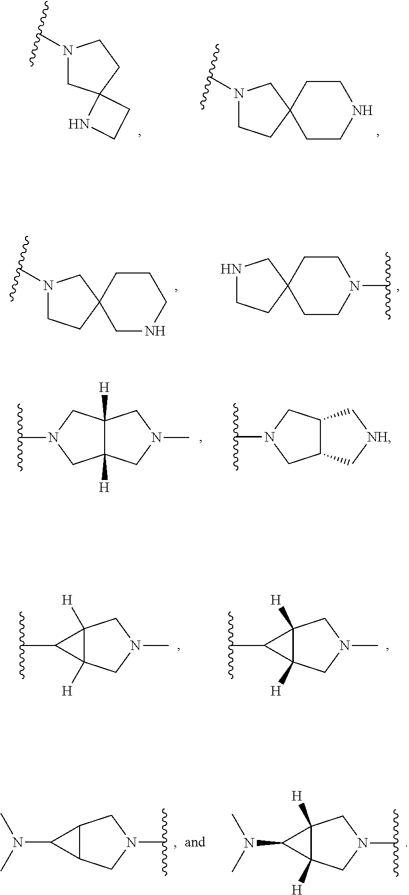

30. The compound of any one of the preceding claims, wherein one of A and B is independently selected from

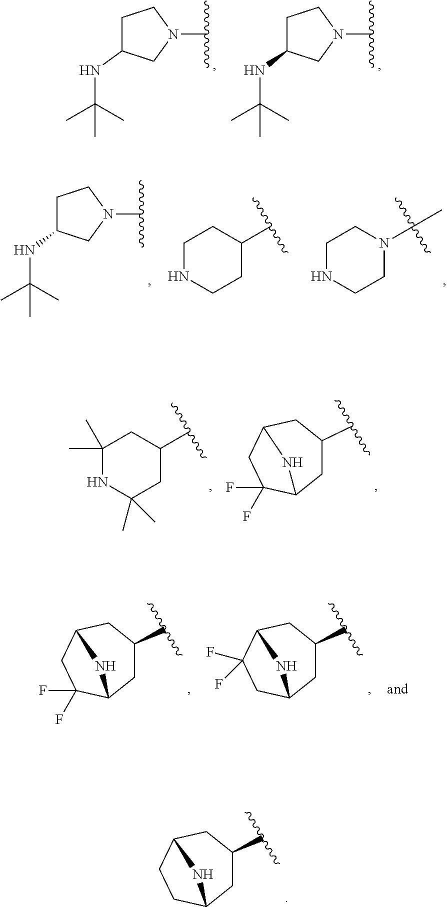

31. The compound of any one of the preceding claims, wherein one of A and B is independently selected from

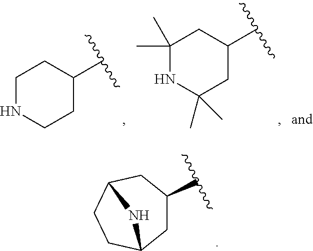

32. The compound of any one of the preceding claims, wherein A is selected from NH,

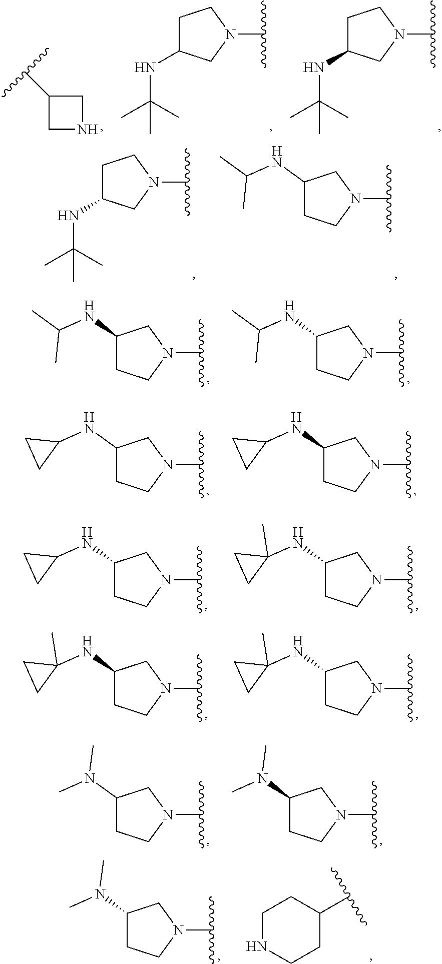

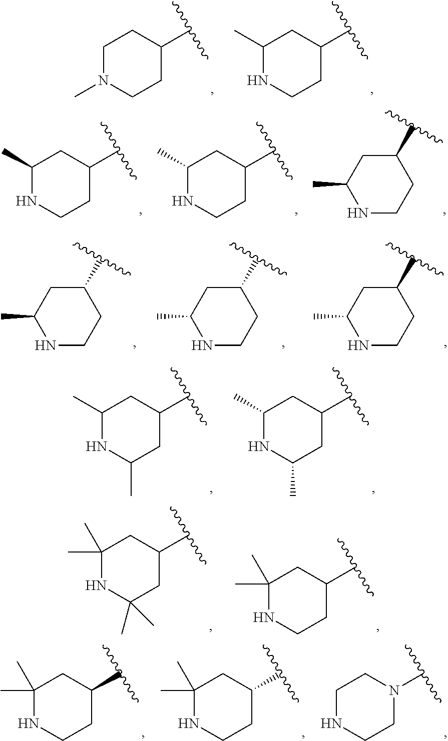

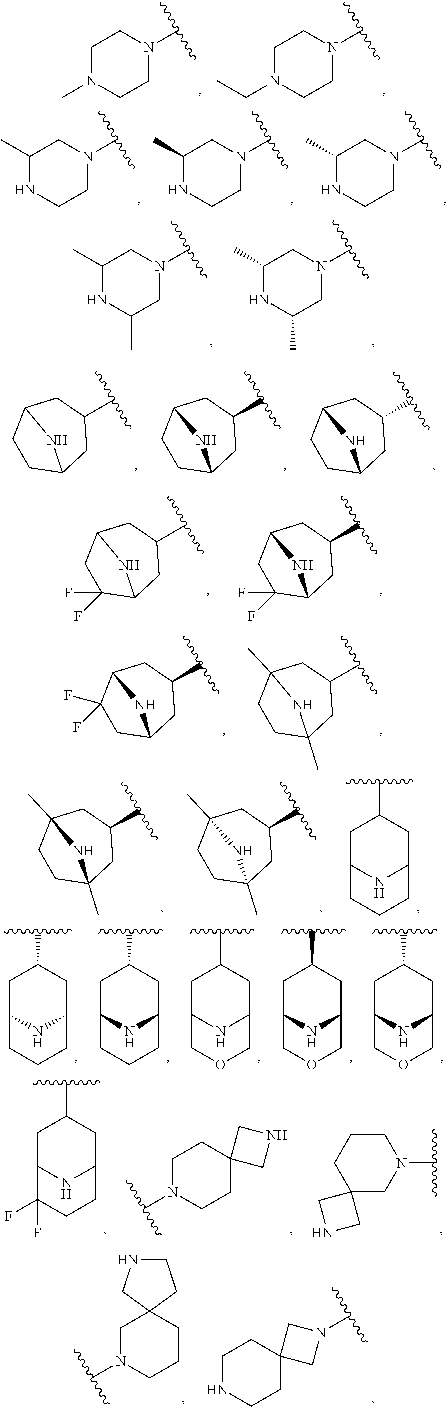

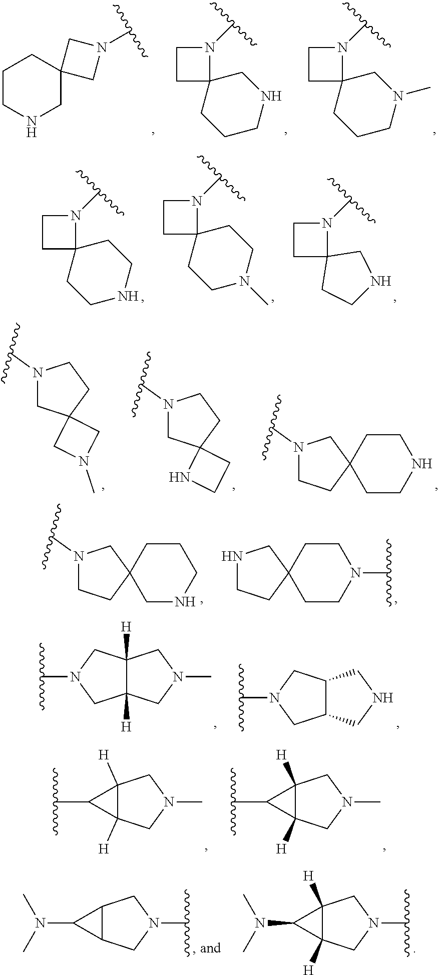

33. The compound of any one of the preceding claims, wherein B is selected from

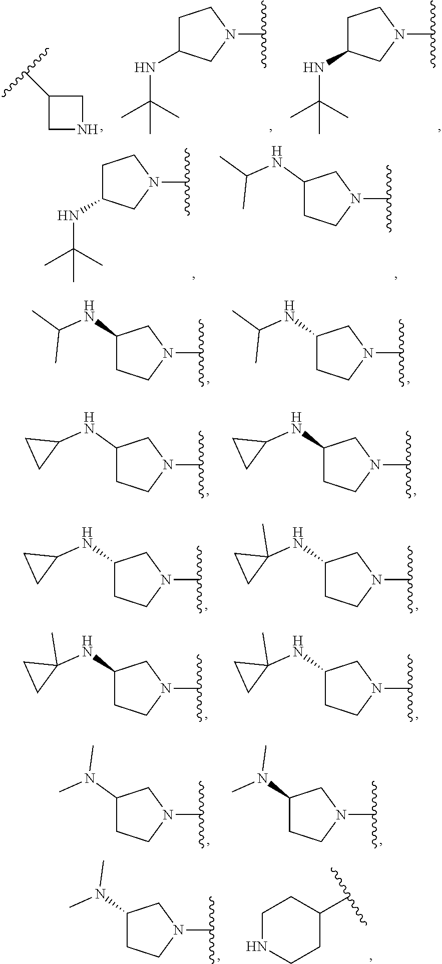

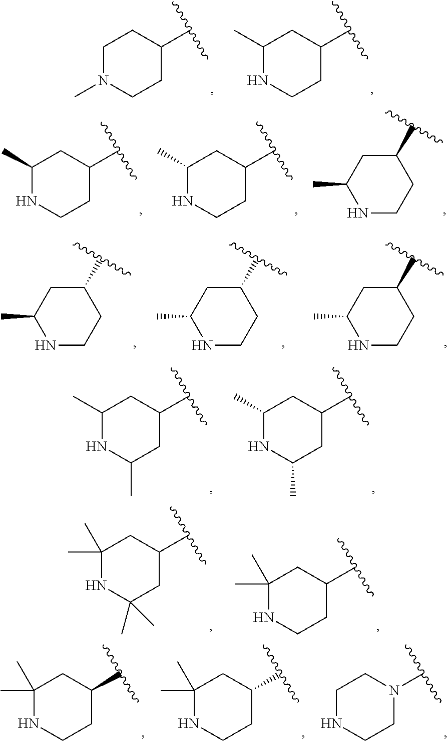

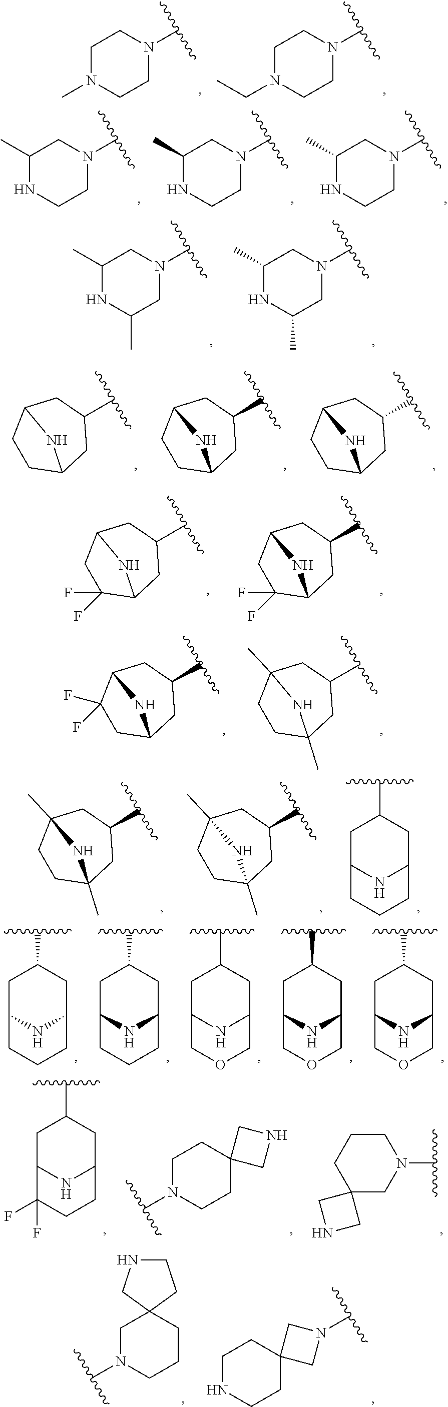

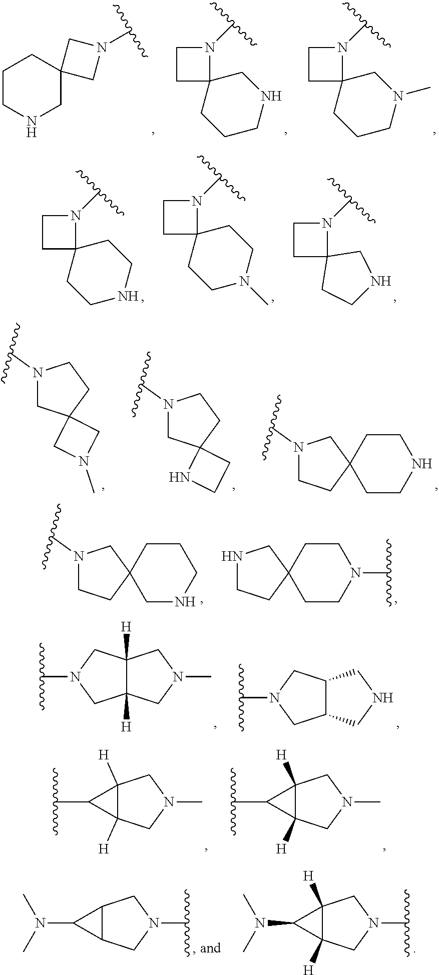

34. The compound of any one of the preceding claims, wherein A is selected from

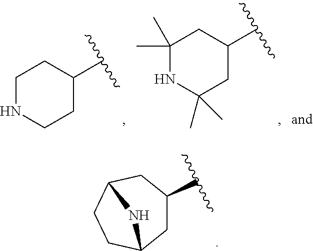

35. The compound of any one of the preceding claims, wherein L is absent, —O—, or —N(R⁴)—.

36. The compound of any one of the preceding claims, wherein L is absent.

37. The compound of any one of the preceding claims, wherein L is —O— or —N(R⁴)—.

38. The compound of any one of the preceding claims, wherein L is —N(R⁴)— and R⁴ is hydrogen, C₁-C₆-alkyl, or cycloalkyl, wherein each alkyl and heteroalkyl are each optionally substituted with one or more R¹² (e.g., deuterium).

39. The compound of any one of the preceding claims, wherein L is —N(CH₃)— or —N(H)—.

40. The compound of any one of the preceding claims, wherein L is —N(CH₃)—.

41. The compound of any one of the preceding claims, wherein three of Z¹, Z², Z³, Z⁴, Z⁵, and Z⁶ are independently C(R⁶) (e.g., CH).

42. The compound of any one of the preceding claims, wherein four of Z¹, Z², Z³, Z⁴, Z⁵, and Z⁶ are independently C(R⁶) (e.g., CH).

43. The compound of any one of the preceding claims, wherein five of Z¹, Z², Z³, Z⁴, Z⁵, and Z⁶ are independently C(R⁶) (e.g., CH).

44. The compound of any one of the preceding claims, wherein each of Z¹, Z², Z³, Z⁴, Z⁵, and Z⁶ is independently C(R⁶) (e.g., CH).

45. The compound of any one of the preceding claims, wherein one of Z¹, Z², Z³, Z⁴, Z⁵, and Z⁶ is independently N.

46. The compound of any one of the preceding claims, wherein two of Z¹, Z², Z³, Z⁴, Z⁵, and Z⁶ are independently N.

47. The compound of any one of the preceding claims, wherein one of Z¹, Z², and Z⁵ is each independently N.

48. The compound of any one of the preceding claims, wherein Z² is N.

49. The compound of any one of the preceding claims, wherein Z² and Z⁵ are each independently N.

50. The compound of any one of claims 1-48, wherein Z¹ and Z² are each independently N.

51. The compound of any one of the preceding claims, wherein one of X and Y is C(R^{7a})(R^{7b}), and the other of X and Y is O.

52. The compound of any one of the preceding claims, wherein X is O and Y is CH₂.

53. The compound of any one of claims 1-51, wherein X is CH₂ and Y is O.

54. The compound of any one of the preceding claims, wherein n and m are both 1.

55. The compound of any one of claims 1-53, wherein n is 1 and m is 2.

56. The compound of any one of claims 1-53, wherein n is 2 and m is 1.

57. The compound of any one of the preceding claims, wherein 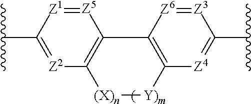
is selected from 0

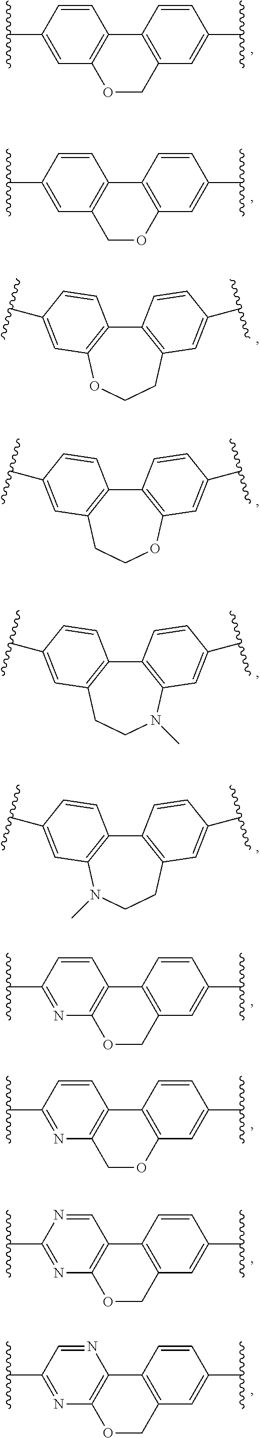

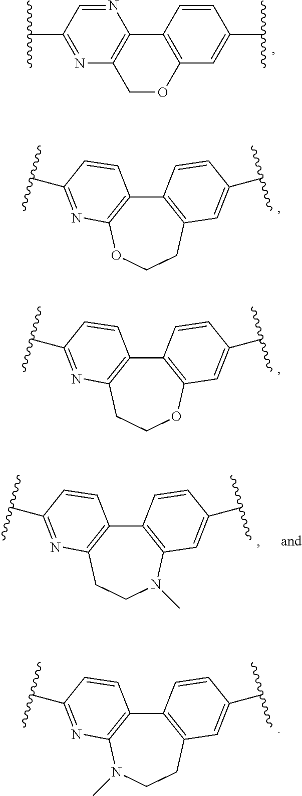 [SMILES: *c1ccc2c(c1)OCc1nc(*)cnc1-2 ; *c1ccc2c(c1)CCOc1nc(*)ccc1-2 ; *c1ccc2c(c1)OCCc1nc(*)ccc1-2 ; *c1ccc2c(c1)N(C)CCc1nc(*)ccc1-2 ; *c1ccc2c(c1)CCN(C)c1nc(*)ccc1-2]

58. The compound of any one of the preceding claims, wherein 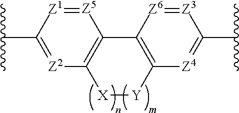
is selected from

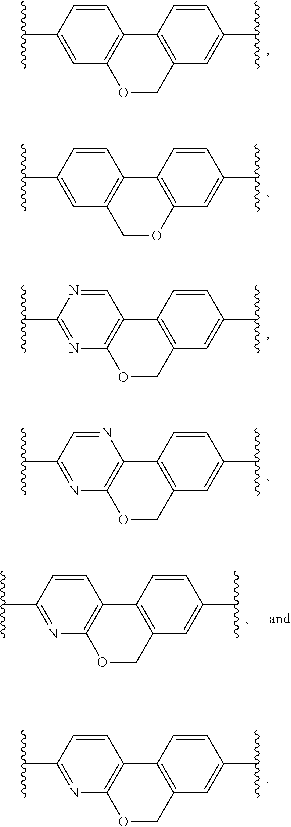 [SMILES: *c1ccc2c(c1)COc1cc(*)ccc1-2 ; *c1ccc2c(c1)COc1cc(*)ccc1-2 ; *c1ccc2c(c1)COc1nc(*)ncc1-2 ; *c1ccc2c(c1)COc1nc(*)cnc1-2 ; *c1ccc2c(c1)COc1nc(*)ccc1-2 ; *c1ccc2c(c1)COc1nc(*)ccc1-2]

59. The compound of any one of the preceding claims, wherein 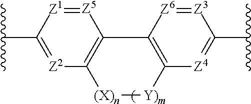
is selected from

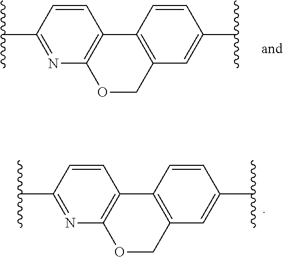 [SMILES: *c1ccc2c(c1)COc1nc(*)ccc1-2 ; *c1ccc2c(c1)COc1nc(*)ccc1-2]

60. The compound of any one of the preceding claims, wherein the compound of Formula (I) is a compound of Formula (I-a): 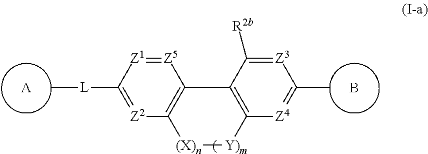
or a pharmaceutically acceptable salt, solvate, hydrate, tautomer, or stereoisomer thereof, wherein R^{2b} is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —OR^{A}, R^{B}R^{C}, —C(O)R^{D}, or —C(O)OR^{D}; and A, B, L, Z¹, Z², Z³, Z⁴, Z⁵, X, Y, m, n, and subvariables thereof are as defined in claim 1.

61. The compound of any one of the preceding claims, wherein the compound of Formula (I) is a compound of Formula (I-b): 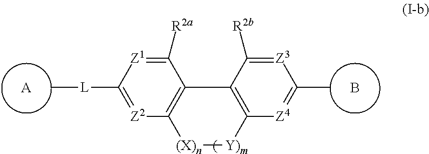
or a pharmaceutically acceptable salt, solvate, hydrate, tautomer, or stereoisomer thereof, wherein R^{2a} and R^{2b} are each independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —OR^{A}, —NR^{B}R^{C}, —C(O)R^{D}, or —C(O)OR^{D}; and A, B, L, Z¹, Z², Z³, Z⁴, X, Y, m, n, and subvariables thereof are as defined in claim 1.

62. The compound of any one of the preceding claims, wherein the compound of Formula (I) is a compound of Formula (I-c): 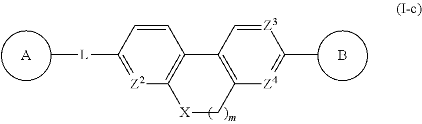
or a pharmaceutically acceptable salt, solvate, hydrate, tautomer, or stereoisomer thereof, wherein A, B, L, Z², Z³, Z⁴, X, m, and subvariables thereof are as defined in claim 1.

63. The compound of any one of the preceding claims, wherein the compound of Formula (I) is a compound of Formula (I-d): 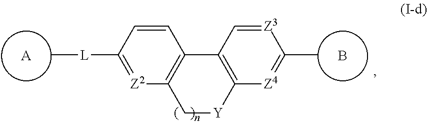
or a pharmaceutically acceptable salt, solvate, hydrate, tautomer, or stereoisomer thereof, wherein A, B, L, Z², Z³, Z⁴, Y, m, and subvariables thereof are as defined in claim 1.

64. The compound of any one of the preceding claims, wherein the compound of Formula (I) is a compound of Formula (I-e): 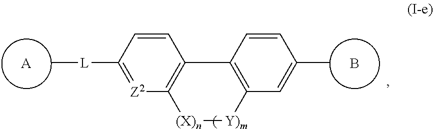
or a pharmaceutically acceptable salt, solvate, hydrate, tautomer, or stereoisomer thereof, wherein A, B, L, Z², X, Y, m, n, and subvariables thereof are as defined in claim 1.

65. The compound of any one of the preceding claims, wherein the compound of Formula (I) is a compound of Formula (I-f): 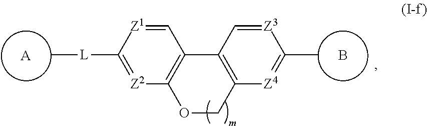
or a pharmaceutically acceptable salt, solvate, hydrate, tautomer, or stereoisomer thereof, wherein A, B, L, Z¹, Z², Z³, Z⁴, m, and subvariables thereof are as defined in claim 1.

66. The compound of claim 65, wherein A is heterocyclyl and B is heteroaryl.

67. The compound of any one of claims 65-66, wherein L is absent or N(R⁴); Z² is N; each of Z¹, Z², Z³, and Z⁴ is independently C(R⁶); and m is 1.

68. The compound of any one of the preceding claims, wherein the compound of Formula (I) is a compound of Formula (I-g): 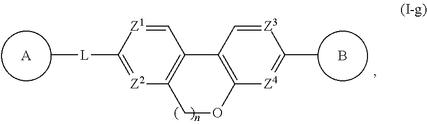
or a pharmaceutically acceptable salt, solvate, hydrate, tautomer, or stereoisomer thereof, wherein A, B, L, Z¹, Z², Z³, Z⁴, n, and subvariables thereof are as defined in claim 1.

69. The compound of any one of the preceding claims, wherein compound of Formula (I) is a compound of Formula (I-h): 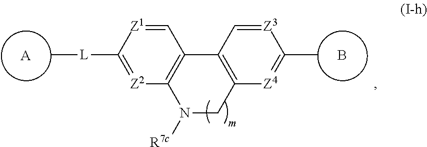
or a pharmaceutically acceptable salt, solvate, hydrate, tautomer, or stereoisomer thereof, wherein A, B, L, Z¹, Z², Z³, Z⁴, R^{7c}, m, and subvariables thereof are as defined in claim 1.

70. The compound of any one of the preceding claims, wherein the compound is selected from any one of the compounds shown in Table 1 or a pharmaceutically acceptable salt, solvate, hydrate, tautomer, or stereoisomer thereof.

71. A pharmaceutical composition comprising a compound of any one of claims 1-70 and a pharmaceutically acceptable excipient.

72. The compound of any one of claims 1-70 or the pharmaceutical composition of claim 71, wherein the compound alters a target nucleic acid (e.g., an RNA, e.g., a pre-mRNA).

73. The compound of any one of claims 1-70 or the pharmaceutical composition of claim 71, wherein the compound binds to a target nucleic acid (e.g., an RNA, e.g., a pre-mRNA).

74. The compound of any one of claims 1-70 or the pharmaceutical composition of claim 71, wherein the compound stabilizes a target nucleic acid (e.g., an RNA, e.g., a pre-mRNA).

75. The compound of any one of claims 1-70 or the pharmaceutical composition of claim 71, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9% 10%, 15%, 20%, 25%, 30%, 35%, 40%, 45%, 50%, 55%, 60%, 65%, 70%, 75%, 80%, 85%, 90%, 95%, or more, e.g., as determined by qPCR.

76. The compound of any one of claims 1-70 or the pharmaceutical composition of claim 71, wherein the compound decreases splicing at splice site on a target nucleic acid (e.g., an RNA, e.g., a pre-mRNA), by about 0.5%, 1%, 2%, 3% 4%, 5%, 6%, 7%, 8%, 9% 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

77. A method of modulating splicing of a nucleic acid (e.g., DNA, RNA, e.g., a pre-mRNA) comprising contacting the nucleic acid with a compound of Formula (I) as described in any one of claims 1-70.

78. The method of claim 77, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

79. The method of claim 77, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%0, 40%, 45%, 50%, 55%, 60%, 65%, 70%, 75%, 80%, 85%, 90%, 95%, or more, e.g., as determined by qPCR.

80. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), comprising contacting the nucleic acid (e.g., a DNA, RNA, e.g., a pre-mRNA) with a compound of Formula (I) as described in any one of claims 1-77.

81. The method of claim 80, wherein the component of a spliceosome is recruited to the nucleic acid in the presence of the compound of Formula (I).

82. A method of altering the conformation of a nucleic acid (e.g., a DNA, RNA, e.g., a pre-mRNA) comprising contacting the nucleic acid with a compound of Formula (I) as described in any one of claims 1-70.

83. The method of claim 82, wherein the altering comprises forming a bulge in the nucleic acid.

84. The method of claim 82, wherein the altering comprises stabilizing a bulge in the nucleic acid.

85. The method of claim 82, wherein the altering comprises reducing a bulge in the nucleic acid.

86. The method of any one of claims 82-85, wherein the nucleic acid comprises a splice site.

87. A method for treating a disease or disorder in a subject comprising administering to the subject a compound of Formula (I) according to any one of claims 1-70 or the pharmaceutical composition of claim 71.

88. The method of claim 87, wherein the disease or disorder comprises a proliferative disease (e.g., cancer, a benign neoplasm, or angiogenesis).

89. The method of any one of claims 87-88, wherein the disease or disorder comprises cancer.

90. The method of claim 89, wherein the cancer is selected form adenoid cystic carcinoma, colorectal cancer, leukemia, lung cancer, prostate cancer, breast cancer, or ovarian cancer.

91. The method of claim 87, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

92. The method of claim 91, wherein the disease or disorder comprises neurological disease or disorder.

93. The method of claim 92, wherein the disease or disorder comprises Huntington's disease.

94. A composition for use in treating a disease or disorder in a subject comprising a compound of Formula (I) according to any one of claims 1-70 or the pharmaceutical composition of claim 71.

95. The composition for use of claim 94, wherein the disease or disorder comprises a proliferative disease (e.g., cancer, a benign neoplasm, or angiogenesis).

96. The composition for use of any one of claims 94-95, wherein the disease or disorder comprises cancer.

97. The composition for use of claim 96, wherein the cancer is selected form adenoid cystic carcinoma, colorectal cancer, leukemia, lung cancer, prostate cancer, breast cancer, or ovarian cancer.

98. The composition for use of claim 97, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

99. The composition for use of claim 98, wherein the disease or disorder comprises neurological disease or disorder.

100. The composition for use of claim 99, wherein the disease or disorder comprises Huntington's disease
